(19) United States
(12) Patent Application Publication
(10) Pub. No.: US 2025/0270575 A1
(43) Pub. Date: Aug. 28, 2025

(54) COMPOSITIONS AND METHODS FOR INDOOR AIR REMEDIATION

(71) Applicant: Neoplants SAS, Saint Denis (FR)

(72) Inventor: Patrick Torbey, Saint-Ouen (FR)

(21) Appl. No.: 18/284,959

(22) PCT Filed: Apr. 7, 2022
(86) PCT No.: PCT/EP2022/059345

§ 371 Date: Sep. 29, 2023

Related U.S. Application Data
(60) Provisional application No. 63/171,872, filed on Apr. 7, 2021.

(51) Int. Cl. C12N 15/82; A01H 1/04; B01D 53/34; C12N 1/20; C12R 1/01

(52) U.S. Cl. CPC C12N 15/8259; A01H 1/04; B01D 53/34; C12N 1/20; B01D 2251/95; B01D 2257/708; C12R 2001/01

(57) ABSTRACT

The present disclosure provides compositions, methods of use, and methods of creation for a population of transgenic plants derived from plant cells transformed with recombinant DNA for expression of heterologous proteins. In particular, the present disclosure provides compositions comprising indoor ornamental plants suited for the removal of volatile organic compounds such as formaldehyde, benzene, toluene, ethylbenzene and/or xylene from air. Also disclosed are transgenic seeds for growing a transgenic plant having the recombinant DNA in its genome and exhibiting enhanced VOC removal from air. Also disclosed are methods for generating seed and plants based on the transgenic events. Also disclosed are microbes selected for during directed evolution to have enhanced VOC removal from air capabilities. Also disclosed are methods and compositions for generating plant-microbiome pairings for enhanced VOC removal from air.

### CROSS-REFERENCE TO RELATED APPLICATIONS

This application claims priority to U.S. Provisional Application No. 63/171,872 filed Apr. 7, 2021, the entirety of each of which is incorporated herein by reference.

### BACKGROUND

Indoor air contamination is a complex and ubiquitous problem, involving particles (such as dust and smoke), biological agents (molds, spores), radon, asbestos, and gaseous contaminants such as CO, CO₂, NOₓ, SOₓ, aldehydes and Volatile Organic Compounds (VOCs). Many of these particulates have been directly linked to disease states or are strongly suspected to cause disease. Compounds such as VOCs are thought to cause many Indoor Air Quality (IAQ) associated health problems and potentially “sick-building syndrome” symptoms. As such, there is a pressing need for the creation and production of compositions and methods suitable for purifying indoor air.

### SUMMARY

The present disclosure provides technologies for improving indoor air quality. Among other things, the present disclosure provides an insight that certain ornamental plants can be engineered and/or cultivated to improve air quality, for example, through removal of VOCs and/or other agents from the air.

In some embodiments, provided technologies include and/or utilize engineered proteins (e.g., enzymes that capture and/or detoxify air-borne agents), genes, plants, and/or microorganisms (e.g., in the plant biome) and/or technologies for developing, producing, and/or utilizing them. In some embodiments, provided technologies includes systems (e.g., methods and/or components) for cultivating plants and/or associated organisms (e.g., microorganisms for example that may participate in a plant microbiome.

In some embodiments, the present disclosure provides an insight that a multifactorial approach to improving indoor air quality may be particularly useful, among other things because such a strategy effectively purify air, while avoiding single point failures.

In some embodiments, provided technologies enhance pollutant entry rate inside a plant through increased stomatal conductance. Alternatively or additionally, in some embodiments, provided technologies engineer optimized synthetic degradation pathways inside plant(s). Still further alternatively or additionally, in some embodiments, the present disclosure provides technologies for increasing depolluting capacity of a plant's microbiome.

Among the advantages achieved by embodiments of technologies provided herein are dramatically augmented phytoremediation efficiency of indoor plants. In some embodiments, a single potted neoplant as described herein can achieve VOC removal effectiveness comparable or superior to that typically observed with a traditional biowall.

In some embodiments, provided technologies include an engineered ornamental indoor plant characterized in that: (a) it expresses at least one (heterologous) formaldehyde and/or methanol metabolism polypeptide: and (b) when cultivated in an environment comprising a volatile organic compound (VOC), exhibits an increased rate of air VOC removal, when compared to an ornamental indoor plant that has not been so engineered.

In some embodiments, provided technologies include an engineered ornamental indoor plant that is stably transformed with at least one expression vector from which the at least one formaldehyde metabolism polypeptide is expressed. In some embodiments, provided technologies comprise a plurality of formaldehyde metabolism polypeptides that are expressed from at least one expression vector. Further still, in some embodiments, provided technologies comprise a plurality of expression vectors from which a plurality of formaldehyde metabolism polypeptides are expressed. In some embodiments, provided technologies comprise a plurality of polypeptides that are designed to function in concert to chemically convert a VOC to a usable sugar substrate.

In some embodiments, provided technologies comprise an engineered ornamental indoor plant expressing at least one heterologous formaldehyde metabolism polypeptide. In some embodiments, a provided heterologous formaldehyde metabolism polypeptide comprises: 3-hexulose-6-phosphate synthase (HPS), 6-phospho-3-hexuloisomerase (PHI), dihydroxyacetone synthase (DAS), dihydroxyacetone kinase (DAK), formaldehyde dehydrogenase (FALDH), glutathione-dependent formaldehyde dehydrogenase (GSH-FALDH), glycolaldehyde synthase (GALS), acetyl-phosphate synthase (ACPS), phosphate acetyltransferase (PTA), 2-keto-4-hydroxybutyrate aldolase (KHB), branched-chain alpha-keto acid decarboxylase (KDC), pyruvate decarboxylase (PDC), NADH-dependent 1,3-PDO oxidoreductase (DhaT), non-specific NADPH-dependent alcohol dehydrogenase (YqhD), serine aldolase (SAL), threonine aldolase (LtaE), serine deaminase (SDA), 4-hydroxy-2-oxobutanoate (HOB) aldolase (HAL), HOB aminotransferase (HAT), serine hydroxymethyltransferase 1 mitochondrial (SHM1), (S)-2-hydroxy-acid oxidase (GLO1 and/or GLO2), formate dehydrogenase (FDH), and/or formolase (FLS).

In some embodiments, provided technologies comprise at least one heterologous formaldehyde metabolism polypeptide, wherein the polypeptide comprises 3-hexulose-6-phosphate synthase (HPS), and/or 6-phospho-3-hexuloisomerase (PHI). In some embodiments, provided technologies comprise at least one heterologous formaldehyde metabolism polypeptide, wherein the polypeptide comprises dihydroxyacetone synthase (DAS), and/or dihydroxyacetone kinase (DAK). In some embodiments, provided technologies comprise at least one heterologous formaldehyde metabolism polypeptide, wherein the polypeptide comprises formaldehyde dehydrogenase (FALDH), glutathione-dependent formaldehyde dehydrogenase (GSH-FALDH), serine hydroxymethyltransferase 1 mitochondrial (SHM1), (S)-2-hydroxy-acid oxidase (GLO1 and/or GLO2) and/or formate dehydrogenase (FDH). In some embodiments, provided technologies comprise at least one heterologous formaldehyde metabolism polypeptide, wherein the polypeptide comprises formolase (FLS), and/or dihydroxyacetone kinase (DAK). In some embodiments, provided technologies comprise at least one heterologous formaldehyde metabolism polypeptide, wherein the polypeptide comprises glycolaldehyde synthase (GALS), acetyl-phosphate synthase (ACPS), and/or phosphate acetyltransferase (PTA). In some embodiments, provided technologies comprise at least one heterologous formaldehyde metabolism polypeptide, wherein the polypeptide comprises 2-keto-4-hydroxybutyrate aldolase (KHB), branched-chain alpha-keto acid decarboxylase (KDC), pyruvate decarboxylase (PDC), NADH-dependent 1,3-PDO oxidoreductase (DhaT), and/or non-specific NADPH-dependent alcohol dehydrogenase (YqhD). In some embodiments, provided technologies comprise at least one heterologous formaldehyde metabolism polypeptide, wherein the polypeptide comprises serine aldolase (SAL), threonine aldolase (LtaE), serine deaminase (SDA), 4-hydroxy-2-oxobutanoate (HOB) aldolase (HAL), and/or HOB aminotransferase (HAT).

In some embodiments, provided technologies comprise an engineered ornamental indoor plant expressing at least one heterologous formaldehyde metabolism polypeptide, wherein prior to introduction to the ornamental indoor plant, the at least one heterologous formaldehyde metabolism polypeptide has been modified using protein evolution.

In some embodiments, provided technologies comprise a cell or a population of cells derived from an engineered ornamental indoor plant expressing at least one heterologous formaldehyde metabolism polypeptide.

In some embodiments, provided technologies comprise an engineered ornamental indoor plant characterized in that: (a) it expresses at least one (heterologous) benzene, toluene, ethylbenzene, or xylene (BTEX) metabolism polypeptide: and (b) when cultivated in an environment comprising a volatile organic compound (VOC), exhibits an increased rate of air VOC removal when compared to an ornamental indoor plant that has not been so engineered.

In some embodiments, provided technologies comprise an engineered ornamental indoor plant that is stably transformed with at least one expression vector from which at least one BTEX metabolism polypeptide is expressed. In some embodiments, provided technologies comprise an engineered ornamental indoor plant that is stably transformed with a plurality of expression vectors from which a plurality of BTEX metabolism polypeptides are expressed. In some embodiments, provided technologies comprise an engineered ornamental indoor plant that is stably transformed with a plurality of polypeptides that are designed to function in concert to chemically convert BTEX to a usable anabolic substrate.

In some embodiments, provided technologies comprise an engineered ornamental indoor plant that is stably transformed with at least one expression vector from which at least one BTEX metabolism polypeptide, wherein the at least one heterologous BTEX metabolism polypeptide comprises: cytochrome P450 monooxygenase, O-xylene monooxygenase oxygenase subunit alpha, benzene monooxygenase oxygenase subunit, toluene-4-monooxygenase system ferredoxin-NAD (+) reductase component, toluene monooxygenase alpha subunit, aromatic ring-hydroxylating dioxygenase subunit alpha, hydroxylase alpha subunit, phenylalanine hydroxylase, benzene 1,2-dioxygenase, cis-1,2-dihydrobenzene-1,2-diol dehydrogenase, toluene methyl-monooxygenase, aryl-alcohol dehydrogenase, benzaldehyde dehydrogenase (NAD+), and/or benzaldehyde dehydrogenase (NADP+).

In some embodiments, provided technologies comprise an engineered ornamental indoor plant transformed with at least one heterologous polypeptide that alters the benzene and/or ethylbenzene metabolism pathway, wherein the heterologous polypeptide comprises benzene monooxygenase oxygenase subunit, benzene 1,2-dioxygenase, and/or cis-1,2-dihydrobenzene-1,2-diol dehydrogenase.

In some embodiments, provided technologies comprise an engineered ornamental indoor plant transformed with at least one heterologous polypeptide that alters the toluene and xylene metabolism pathway, wherein the heterologous polypeptide comprise O-xylene monooxygenase oxygenase subunit alpha, toluene-4-monooxygenase system ferredoxin-NAD (+) reductase component, toluene monooxygenase alpha subunit, toluene methyl-monooxygenase, aryl-alcohol dehydrogenase, benzaldehyde dehydrogenase (NAD+) and/or benzaldehyde dehydrogenase (NADP+).

In some embodiments, provided technologies comprise an engineered ornamental indoor plant transformed with at least one heterologous polypeptide that alters phenol and/or phenol (like) metabolism pathways, wherein the heterologous polypeptides comprise phenol hydroxylase component phP, phenol hydroxylase, and/or uncharacterized protein A4U43_C04F5180.

In some embodiments, provided technologies comprise an engineered ornamental indoor plant transformed with at least one heterologous polypeptide that alters catechol and/or catechol (like) metabolism pathways, wherein the heterologous polypeptides comprise 3-isopropylcatechol-2,3-dioxygenase, metapyrocatechase, extradiol dioxygenase, catechol 2,3-dioxygenase, and/or catechol 1,2-dioxygenase.

In some embodiments, provided technologies comprise an engineered ornamental indoor plant, wherein prior to introduction to the ornamental indoor plant, at least one heterologous BTEX metabolism polypeptide has been modified using protein evolution.

In some embodiments, provided technologies comprise a cell or a population of cells derived from an engineered ornamental indoor plant expressing at least one heterologous BTEX metabolism polypeptide.

In some embodiments, provided technologies comprise an engineered ornamental indoor plant created by crossing an engineered ornamental plant comprising at least one heterologous formaldehyde metabolism pathway polypeptide with an engineered ornamental plant comprising at least one heterologous BTEX metabolism pathway polypeptide. In some embodiments, provided technologies comprise an engineered ornamental indoor plant comprising at least one heterologous formaldehyde metabolism pathway polypeptide and at least one heterologous BTEX metabolism polypeptide. In some embodiments, provided technologies comprise a cell or population of cells derived from the engineered ornamental indoor plant comprising at least one heterologous formaldehyde metabolism pathway polypeptide and at least one heterologous BTEX metabolism polypeptide.

In some embodiments, provided technologies comprise an engineered ornamental indoor plant characterized in that: (a) at least one pathway related to diffusion and/or active transport of VOCs into the ornamental plant are modified; and (b) when cultivated in an environment comprising a volatile organic compound (VOC), exhibits an increased rate of air VOC removal when compared to an ornamental indoor plant that has not been modified.

In some embodiments, provided technologies comprise an engineered ornamental indoor plant that is stably transformed with at least one expression vector from which at least one polypeptide related to pathways regulating diffusion and/or active transport of VOCs into the ornamental plant is expressed. In some embodiments, provided technologies comprise an engineered ornamental indoor plant that is stably engineered to have at least one endogenous polypeptide involved in a pathway related to diffusion and/or active transport of VOCs into the ornamental plant modified. In some embodiments, provided technologies comprise an engineered ornamental indoor plant that is stably engineered to have at least one endogenous polypeptide involved in a pathway related to diffusion and/or active transport of VOCs into the ornamental plant knocked-out, silenced, and/or rendered hypomorphic.

In some embodiments, provided technologies comprise an engineered ornamental indoor plant that is stably engineered to have at least one endogenous polypeptide involved in transgene silencing knocked-out, silenced, and/or rendered hypomorphic. In some embodiments, a polypeptide involved in transgene silencing that is knocked-out, silenced, and/or rendered hypomorphic is RDR6.

In some embodiments, provided technologies comprise an engineered ornamental indoor plant that is stably transformed with at least one expression vector from which at least one polypeptide related to pathways regulating diffusion and/or active transport of VOCs is expressed. In some embodiments, provided technologies comprise an engineered ornamental indoor plant that is stably engineered to have at least one endogenous polypeptide related to stomatal flux knocked-out, silenced, and/or rendered hypomorphic, wherein the at least one polypeptide Epidermal Patterning Factor 1 (EPF1) and/or Epidermal Patterning Factor 2 (EPF2).

In some embodiments, provided technologies comprise an engineered ornamental indoor plant that is stably transformed with at least one expression vector from which at least one polypeptide related to stomatal flux is expressed, wherein the at least one polypeptide comprises Epidermal Patterning Factor-Like protein 9 (EPFL9) (STOMAGEN). In some embodiments, provided technologies comprise an engineered ornamental indoor plant that is stably transformed with at least one expression vector from which at least one polypeptide related to cuticle wax levels is expressed, wherein the at least one polypeptide comprises Aledehyde Decarbonylase (CER1), Fatty Acid Reductase (CER3), Beta-ketoacyl-coenzyme A Synthase, 3′-5′-exoribonuclease family protein (CER7), and/or WOOLLY. In some embodiments, provided technologies comprise an engineered ornamental indoor plant stably transformed with at least one expression vector from which at least one polypeptide related to trichome development is expressed, wherein the at least one polypeptide comprises MYB123-Like, Caprice (CPC), GLABRA1, GLABRA2, and/or GLABRA3. In some embodiments, provided technologies comprise an engineered ornamental indoor plant that is stably transformed with at least one expression vector from which at least one heterologous polypeptide related to active transport of VOCs is expressed, wherein the at least one polypeptide comprises an Oxalate: Formate Antiport polypeptide, Formate: Nitrite Transporter polypeptide, and/or 2FoCA-Anion Channel polypeptide. In some embodiments, provided technologies comprise an engineered ornamental indoor plant wherein prior to introduction to the ornamental indoor plant, at least one polypeptide involved in a pathway related to diffusion and/or active transport of VOCs has been modified using protein evolution.

In some embodiments, provided technologies comprise an engineered ornamental indoor plant created by crossing two engineered ornamental indoor plants. In some embodiments, provided technologies comprise an engineered ornamental plant comprising at least one heterologous formaldehyde metabolism pathway polypeptide and at least one mutation and/or transgenic vector related to stomatal flux. In some embodiments, provided technologies comprise a cell or population of cells derived from the engineered ornamental indoor plant comprising at least one heterologous BTEX metabolism polypeptide and at least one mutation and/or transgenic vector related to stomatal flux. In some embodiments, provided technologies comprise an engineered ornamental indoor plant comprising at least one heterologous formaldehyde metabolism pathway polypeptide, at least one heterologous BTEX metabolism polypeptide, and at least one mutation and/or transgenic vector related to stomatal flux.

In some embodiments, provided technologies comprise an engineered ornamental plant comprising at least one heterologous formaldehyde metabolism pathway polypeptide, and at least one mutation and/or transgenic vector related to inhibition of transgene silencing. In some embodiments, provided technologies comprise an engineered ornamental plant comprising at least one heterologous BTEX metabolism pathway polypeptide, and at least one mutation and/or transgenic vector related to inhibition of transgene silencing. In some embodiments, provided technologies comprise an engineered ornamental plant comprising at least one mutation and/or transgenic vector related to stomatal flux, and at least one mutation and/or transgenic vector related to inhibition of transgene silencing.

In some embodiments, provided technologies comprise an engineered ornamental plant comprising at least one heterologous formaldehyde metabolism pathway polypeptide, at least one mutation and/or transgenic vector related to stomatal flux, and at least one mutation and/or transgenic vector related to inhibition of transgene silencing. In some embodiments, provided technologies comprise an engineered ornamental plant comprising at least one heterologous formaldehyde metabolism pathway polypeptide, at least one heterologous BTEX metabolism polypeptide, at least one mutation and/or transgenic vector related to stomatal flux, and at least one mutation and/or transgenic vector related to inhibition of transgene silencing.

In some embodiments, provided technologies comprise a cell or population of cells derived from the engineered ornamental indoor plant as described herein.

In some embodiments, provided technologies comprise a population of engineered microbes modified to be more amenable for VOC removal and/or metabolism when compared to a population of non-engineered microbes under otherwise comparable conditions.

In some embodiments, a population of engineered microbes are primarily soil dwelling and comprise microbes of the species: Bacillus metanolcius, Ogataea methanolica, Pseudomonas putida, Phanerochaete chrysosporium, and/or Rugosibacter aromaticivorans.

In some embodiments, a population of engineered microbes are primarily leaf and/or epidermal dwelling and comprise microbes of the species: Methylobacterium oryzae, Methylobacterium extorquens, and/or Paraburkholderia phytofirmans.

In some embodiments, a population of engineered microbes are modified to metabolize formaldehyde with greater efficiency and at a greater capacity than microbes which have not been engineered. In some embodiments, a population of engineered microbes are modified to metabolize BTEX with greater efficiency and at a greater capacity than microbes which have not been engineered. In some embodiments, a population of engineered microbes are modified utilizing horizontal gene transfer from a heterologous microbe that has undergone directed evolution to increase formaldehyde and/or BTEX metabolism.

In some embodiments, a population of engineered microbes are of the species Pseudomonas putida, Methylobacterium oryzae or Methylobacterium extorquens.

In some embodiments, a population of engineered microbes are deposited on an engineered ornamental indoor plant as described herein. In some embodiments, a population of engineered microbes are deposited on an otherwise wild type ornamental indoor plant. In some embodiments, a population of engineered microbes are deposited on an engineered ornamental indoor plant. In some embodiments, a population of engineered microbe are deposited and stably colonize an engineered ornamental indoor plant.

In some embodiments, a population of engineered microbes are of the strain MoCBM20. In some embodiments, a population of engineered microbes are of the strain MePA1. In some embodiments, a population of engineered microbes are of the strain PpF1.

In some embodiments, technologies described herein comprise a plant growth system (e.g., planter) comprising: (a) at least one container comprising at least one cavity suitable for receiving plant growth media and an engineered ornamental plant, and (b) at least one air flow device engineered to provide increased airflow to an engineered ornamental plant.

In some embodiments, technologies described herein comprise a plant growth system (e.g., planter) including at least one drainage system engineered to maintain a desired rhizosphere microbiome a composition. In some embodiments, technologies described herein comprise a plant growth system with an engineered indoor ornamental plant as described herein deposited within. In some embodiments, a plant growth system comprising at least one cavity suitable for receiving plant growth media and an engineered ornamental plant and at least one air flow device engineered to provide increased airflow to an engineered ornamental plant are part of the same physical structure. In some embodiments, technologies described herein comprise at least one container designed to increase relative airflow and/or air exchange between the soil and/or microbiome and a surrounding environment when compared to a control technology. In some embodiments, technologies described herein comprise a plant growth system with at least one container designed to maximize relative airflow and/or air exchange between the soil and/or microbiome and a surrounding environment when compared to a control technology.

In some embodiments, technologies described herein comprise a method of removing at least one VOC from an environment, the method comprising cultivating at least one composition (e.g., an engineered indoor ornamental plant and/or an engineered microbe) in an environment comprising VOCs. In some embodiments, a method of removing at least one VOC from an environment comprises cultivating at least one composition (e.g., an engineered indoor ornamental plant and/or an engineered microbe) in an environment for at least 1 day.

In some embodiments, a method of removing at least one VOC from an environment comprises cultivating at least one composition (e.g., an engineered indoor ornamental plant and/or an engineered microbe) every 100 m³ of space.

In some embodiments, technologies described herein comprise a method of assessing an engineered indoor ornamental plant, microbe, plant-microbe combination, or plant-microbe-plant growth system as described herein, (a) cultivating said engineered plant in a controlled environment comprising a readily detectable and quantifiable concentration of VOCs, and (b) determining the level and rate of change in VOC levels in said controlled environment.

In some embodiments, technologies described herein comprise a method of assessing a vector encoding at least one polypeptide utilized to create an engineered ornamental indoor plant as described herein, comprising (a) expressing said vector in a cell, and (b) determining the transcriptional levels, translational levels, and molecular activity levels of said vector; wherein the step of determining the molecular activity of said vector comprises determining the level of VOC removal and/or metabolism relative to that achieved by an otherwise comparable reference cell under otherwise comparable conditions, which reference cell is not expressing or is not expressing to the same level of at least one polypeptide as the test cell.

In some embodiments, provided technologies are an oligonucleotide for use in creation of an engineered ornamental indoor plant and/or engineered microbe. In some embodiments, provided technologies relate to a method of making at least one oligonucleotide for use in creation of an engineered ornamental indoor plant and/or engineered microbe. In some embodiments, provided technologies relate to a method of making at least one engineered ornamental indoor plant comprising the introduction of at least one vector encoding at least one polypeptide. In some embodiments, provided technologies relate to a method of making at least one vector encoding at least one polypeptide utilized to create an engineered ornamental indoor plant.

### Definitions

The scope of the present disclosure is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Throughout the specification, as is common practice, polynucleotide or polypeptide sequences are typically presented in 5′ to 3′ or N-terminus to C-terminus order, from left to right unless otherwise indicated.

Allele: As used herein, the term “allele” refers to one of two or more existing genetic variants of a specific polymorphic genomic locus.

Amino acid: In its broadest sense, as used herein, the term “amino acid” refers to a compound and/or substance that can be incorporated into a polypeptide chain, e.g., through formation of one or more peptide bonds. In some embodiments, an amino acid has a general structure, e.g., H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to an amino acid, other than standard amino acids, which in some embodiments may be or have been prepared synthetically and in some embodiments may be or have been obtained from a natural source. In some embodiments, an amino acid, including a carboxy- and/or amino-terminal amino acid in a polypeptide, can contain a structural modification as compared with the general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some embodiments, the term “approximately” or “about” refers to a range of values that fall within ±10% (greater than or less than) of a stated reference value unless otherwise stated or otherwise evident from context (except where such number would exceed 100% of a possible value). For example, in some embodiments, the term “approximately” or “about” may encompass a range of values that within 10%, 9%, 8%, 7%, 6%, 5%, 4%, 3%, 2%, 1%, or less of a reference value.

Associated: As used herein, two or more events, conditions, or entities may be described as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Characteristic portion: As used herein, the term “characteristic portion,” can refer to a portion of a substance whose presence (or absence) correlates with presence (or absence) of a particular feature, attribute, or activity of the substance. In some embodiments, a characteristic portion of a substance is a portion that is found in a given substance and in related substances that share a particular feature, attribute or activity, but not in those that do not share the particular feature, attribute or activity. In some embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some embodiments, a characteristic portion may be biologically active.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of a polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share a sequence element. In some embodiments, a characteristic sequence element is a sequence element that is found in all members of a family of polypeptides or nucleic acids, and therefore can be used by those of ordinary skill in the art to define members of the family.

Comparable: As used herein, the term “comparable” refers to two or more agents, entities, situations, sets of conditions, subjects, populations, etc., that may not be identical to one another but that are sufficiently similar to permit comparison there between so that one skilled in the art will appreciate that conclusions may reasonably be drawn based on differences or similarities observed. In some embodiments, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet, G. H. et al., 1992, Science 256:1443-1445, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein.

EXEMPLARY CONSERVATIVE AMINO
ACID SUBSTITUTIONS

| For Amino | | |
|---|---|---|
| Acid | Code | Replace With |
| Alanine | A | D-ala, Gly, Aib, β-Ala, Acp, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, B-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4 or 5-phenylproline, AdaA, |
| | | AdaG, cis-3,4 or 5-phenylproline, Bpa, D-Bpa |
| Proline | P | D-Pro, L-I-thioazolidine-4-carboxylic acid, |
| | | D-or-L-1-oxazolidine-4-carboxylic acid (Kauer, |
| | | U.S. Pat. No. 4,511,390) |
| Serine | S | D-Ser, Thr, D-Thr, allo-Thr, Met, D-Met, Met |
| | | (O), D-Met (O), L-Cys, D-Cys |
| Threonine | T | D-Thr, Ser, D-Ser, allo-Thr, Met, D-Met, Met |
| | | (O), D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

Control: As used herein, the term “control” refers to the art-understood meaning of a “control” being a standard or reference against which results are compared. Typically, controls are used to augment integrity in experiments by isolating variables in order to make a conclusion about such variables. In some embodiments, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some embodiments, a control is a historical control (e.g., of a test or assay performed previously, or an amount or result that is previously known). In some embodiments, a control is or comprises a printed or otherwise saved record. In some embodiments, a control is a positive control. In some embodiments, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some embodiments, “Assaying for the presence of” can be determining an amount of something present and/or determining whether or not it is present or absent.

Engineered: In general, as used herein, the term “engineered” refers to an aspect of having been manipulated by the hand of man. For example, in some embodiments, a cell or organism may be considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity. In some embodiments, a cell or organism may be considered to be “engineered” if it has been handled or cultivated in a manner involving one or more interventions by man.

Expression: As used herein, the term “expression” of a nucleic acid sequence refers to generation of any gene product (e.g., transcript, e.g., mRNA, e.g., polypeptide, etc.) from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, the term “functional” describes something that exists in a form in which it exhibits a property and/or activity by which it is characterized. For example, in some embodiments, a “functional” biological molecule is a biological molecule in a form in which it exhibits a property and/or activity by which it is characterized. In some such embodiments, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some embodiments, a gene may encode a polypeptide, but that polypeptide may not be functional, e.g., a gene variant may encode a polypeptide that does not function in the same way, or at all, relative to the wild-type gene. In some embodiments, a gene may encode a transcript which, in some embodiments, may be toxic beyond a threshold level. In some embodiments, a gene may encode a polypeptide, but that polypeptide may not be functional and/or may be toxic beyond a threshold level.

Heterologous: The term “heterologous”, as used herein to refer to an entity (e.g., a gene or polypeptide) that is present in a different source, in a different arrangement, and/or in a different condition or state from that in which it is presently found. To give but one example, in some embodiments, a gene or polypeptide that is not naturally found in a particular organism is considered to be heterologous to that organism. Alternatively or additionally, in some embodiments, a gene or polypeptide that is not naturally found in a particular cell may be considered to be heterologous to that cell if introduced into it (e.g., via a vector), even if that gene or polypeptide might naturally be found in a different cell of the same type. In some embodiments, a vector may be considered to be heterologous to a cell when it has been introduced into the cell, and/or a copy of a gene included in such vector may be considered to be heterologous to that particular cell even if an endogenous copy of the same gene exists in the cell. Where a plurality of different heterologous polypeptides are to be introduced into and/or expressed by a host cell, different polypeptides may be from different source organisms, or from the same source organism. To give but one example, in some cases, individual polypeptides may represent individual subunits of a complex protein activity and/or may be required to work in concert with other polypeptides in order to achieve the goals of the present invention. In some embodiments, it will often be desirable for such polypeptides to be from the same source organism, and/or to be sufficiently related to function appropriately when expressed together in a host cell. In some embodiments, such polypeptides may be from different, even unrelated source organisms. It will further be understood that, where a heterologous polypeptide is to be expressed in a host cell, it will often be desirable to utilize nucleic acid sequences encoding the polypeptide that have been adjusted to accommodate codon preferences of the host cell and/or to link the encoding sequences with regulatory elements active in the host cell. For example, when the host cell is a Araceae family member (e.g., Epipremnum aureum), it will often be desirable to alter the gene sequence encoding a given polypeptide such that it conforms more closely with the codon preferences of such a Araceae family member. In certain embodiments, a gene sequence encoding a given polypeptide is altered to conform more closely with the codon preference of a species related to the host cell. For example, when the host cell is a Proteobacteria phylum member (e.g., Methylobacterium), it will often be desirable to alter the gene sequence encoding a given polypeptide such that it conforms more closely with the codon preferences of a related bacterial strain. Such embodiments are advantageous when the gene sequence encoding a given polypeptide is difficult to optimize to conform to the codon preference of the host cell due to experimental (e.g., cloning) and/or other reasons. In certain embodiments, the gene sequence encoding a given polypeptide is optimized even when such a gene sequence is derived from the host cell itself (and thus is not heterologous). For example, a gene sequence encoding a polypeptide of interest may not be codon optimized for expression in a given host cell even though such a gene sequence is isolated from the host cell strain. In such embodiments, the gene sequence may be further optimized to account for codon preferences of the host cell. Those of ordinary skill in the art will be aware of host cell codon preferences and will be able to employ inventive methods and compositions disclosed herein to optimize expression of a given polypeptide in the host cell.

Host Cell: As used herein, the “host cell” is a cell (e.g., a plant, fungal, or bacterial cell) that is manipulated according to the present invention, e.g., to receive a vector. In some instances, the term “modified host cell” may be used to refer to a host cell which has been modified, engineered, or manipulated in accordance with the present invention as compared with a parental cell (which may, in some embodiments, be a naturally occurring parental cell or, in other embodiments, may be a parental cell that itself has been engineered or manipulated, including as a host cell). Persons of skill upon reading this disclosure will understand that such terms typically refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4:11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

Isolated: As used herein, the term “isolated”, means that the isolated entity has been separated from at least one component with which it was previously associated. When most other components have been removed, the isolated entity is “purified” or “concentrated”. Isolation and/or purification and/or concentration may be performed using any techniques known in the art including, for example, fractionation, extraction, precipitation, or other separation.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Nucleic acid: As used herein, the term “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments, a nucleic acid has a nucleotide sequence comprising at least one element that encodes, or is complementary to a sequence that encodes, a polypeptide. In some embodiments, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with coding elements of interest; in some embodiments, control elements act in trans to or otherwise at a from the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pathogenic: Those skilled in the art will appreciate that the term “pathogenic” generally refers to an ability to or character of causing disease. In some embodiments, a particular organism or condition may be characterized as or understood to be pathogenic if its presence under relevant circumstances creates a significant and relevant risk of disease to individual(s) who may be present in and/or exposed to the circumstances. Thus, in some embodiments, as will be understood in the art, “pathogenicity” of a particular organism may be impacted by one or more features or elements of context (e.g., amount of organism, size of space, probability of co-localization of organism and potentially susceptible individual, degree of filtration and/or airflow, etc). Alternatively, in some embodiments, an organism may be considered to be “pathogenic” if a material risk of disease would exist if a potentially susceptible individual were exposed to the organism, e.g., under particular standard or experimental or reference conditions.

Phytosphere: The term “phytosphere” will be understood by those skilled in the art to refer to the ecosystem of a plant (e.g., the interior and/or exterior of a plant). In some embodiments, a phytosphere may be or comprise one or more of a phyllosphere, endosphere, and/or rhizosphere.

Polyadenylation: As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. In some embodiments, a 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, a poly(A) tail can be added onto transcripts that contain a specific sequence, the polyadenylation signal or “poly(A) sequence.” A poly(A) tail and proteins bound to it aid in protecting mRNA from degradation by exonucleases. Polyadenylation can be affect transcription termination, export of the mRNA from the nucleus, and translation. Typically, polyadenylation occurs in the nucleus immediately after transcription of DNA into RNA, but additionally can also occur later in the cytoplasm. After transcription has been terminated, the mRNA chain can be cleaved through the action of an endonuclease complex associated with RNA polymerase. The cleavage site can be characterized by the presence of the base sequence AAUAAA near the cleavage site. After mRNA has been cleaved, adenosine residues can be added to the free 3′ end at the cleavage site. As used herein, a “poly(A) sequence” is a sequence that triggers the endonuclease cleavage of an mRNA and the additional of a series of adenosines to the 3′ end of the cleaved mRNA.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Polynucleotide: As used herein, the term “polynucleotide” refers to a polymeric chain of nucleic acids. In some embodiments, a polynucleotide is or comprises RNA; in some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide includes one or more introns. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded; in some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of an ornamental indoor plant, microbiome component, etc).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Regulatory Element: As used herein, the term “regulatory element” or “regulatory sequence” refers to a non-coding region of a nucleic acid (e.g., DNA) that regulates one or more aspects of expression of one or more particular genes. In some embodiments, a regulatory element may act in cis with a gene it regulates. In some embodiments, a regulatory element may act in trans with a gene it regulates. In some embodiments, a regulatory element is apposed to or “in the neighborhood” of a gene that it regulates. In some embodiments, a regulatory element, even if in cis with a gene it regulates, is distinct from the gene. In some embodiments, a regulatory element impairs or enhances transcription of one or more genes. In some embodiments, a regulatory sequence refers to a nucleic acid sequence which is regulates expression of a gene product operably linked to a regulatory sequence. In some such embodiments, this sequence may be an enhancer sequence and other regulatory elements which regulate expression of a gene product.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe (e.g., virus), a plant, or an animal (e.g., a human). In some embodiments, a source of interest is or comprises biological tissue or fluid. In some embodiments, a biological fluid may be or comprise an intracellular fluid, an extracellular fluid, an intravascular fluid, an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab, scraping, surgery, washing or lavage. In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Source organism: The term “source organism”, as used herein, refers to the organism in which a particular agent (e.g., a particular nucleic acid, polypeptide, etc.) can be found in nature. Thus, for example, if one or more heterologous polypeptides is/are being expressed in a host organism, the organism in which the polypeptides are expressed in nature (and/or from which their genes were originally cloned) may be referred to as the “source organism”. Where multiple heterologous polypeptides are being expressed in a host organism, one or more source organism(s) may be utilized for independent selection of each of the heterologous polypeptide(s). It will be appreciated that any and all organisms that naturally contain relevant polypeptide sequences may be used as source organisms in accordance with the present invention. In certain embodiments, representative source organisms may be or include, for example, one or more of animal (e.g., mammal, reptile, fish, bird, insect, etc), plant, microbial (e.g., fungal (e.g., yeast), algal, bacterial [e.g., cyanobacterial, archaebacterial, etc] protozoal, etc) source organisms.

Stomatal Flux: As used herein, the term “stomatal flux” refers to the cycling of a stoma opening, from open-to-closed, or closed-to-open. Stomatal flux may also refer to the propensity for the stoma to appear in one state or the other, e.g., open or closed.

Subject: As used herein, the term “subject” refers an organism (e.g., a plant, a microbe, etc). In many embodiments, where a subject is a plant, it may be an indoor plant, e.g., an ornamental indoor plant. In some embodiments, a plant subject may be in seed form. In some embodiments, a subject can be manipulated (e.g., engineered), for example to better serve a specific purpose.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Variant: As used herein, the term “variant” refers to a version of something, e.g., a gene sequence, that is different, in some way, from another version. To determine if something is a variant, a reference version is typically chosen and a variant is different relative to that reference version. In some embodiments, a variant can have the same or a different (e.g., increased or decreased) level of activity or functionality than a wild type sequence. For example, in some embodiments, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some embodiments, a variant has a reduction or elimination in activity or functionality or a change in activity that results in a negative outcome. Such a variant is referred to herein as a loss-of-function variant. In some embodiments, a gain-of-function variant is a codon-optimized sequence which encodes a transcript or polypeptide that may have improved properties (e.g., less susceptibility to degradation, e.g., less susceptibility to miRNA mediated degradation) than its corresponding wild type (e.g., non-codon optimized) version. In some embodiments, a loss-of-function variant has one or more changes that result in a transcript or polypeptide that is defective in some way (e.g., decreased function, non-functioning) relative to the wild type transcript and/or polypeptide.

Vector: As used herein, the term “vector” refers to a nucleic acid capable of carrying (e.g., into a cell) at least one heterologous polynucleotide with which it has been linked. In some embodiments, a vector can be or comprise a plasmid, a transposon, a cosmid, an artificial chromosome (e.g., a human artificial chromosome (HAC), a yeast artificial chromosome (YAC), a bacterial artificial chromosome (BAC), a P1-derived artificial chromosome (PAC)), a viral vector, a Gateway® plasmid, etc. In certain embodiments, a vector may include sufficient cis-acting elements for expression; alternatively or additionally, elements for expression can be supplied by a cell or system into which the vector is introduced. In some embodiments, a vector may include one or more genetic elements (e.g., origin of replication, primer binding site, etc.) sufficient to achieve replication of the vector in a relevant cell or system. In some embodiments (e.g., bacterial vectors having a bacterial origin of replication and episomal mammalian vectors), a vector may be capable of autonomous replication in a cell or system into which it is introduced. Other vectors (e.g., non-episomal mammalian vectors) can be into nucleic acid(s) already present in such system (e.g., into the genome of a host cell), so that they are replicated along with such present nucleic acid(s). In some embodiments, a vector may be capable of directing expression of genes they carry; such vectors are referred to herein as “expression vectors.”

Volatile Organic Compound: Those of ordinary skill in the art will appreciate that the term “Volatile Organic Compound” (“VOC”) is typically used to refer to compounds that have relatively high vapor pressure and low water solubility. In some embodiments, a VOC may be a carbon-containing compound, excluding carbon monoxide, carbon dioxide, carbonic acid, metallic carbides or carbonates, and ammonium carbonate, which participates in atmospheric photochemical reactions. In some embodiments, a VOC may be or comprise a human made chemical, for example such as may have been used and/or produced in the manufacture of an entity such as a paint, a varnish, a wax, a pharmaceutical, a refrigerant, a cleaning or disinfecting product, a degreasing product, a fuel, etc. Alternatively or additionally, in some embodiments, a VOC may be or comprise a solvent, e.g., an industrial solvent (e.g., trichloroethylene), a fuel oxygenates (e.g., methyl tert-butyl ether (MTBE)), a by-product produced by chlorination in water treatment (e.g., chloroform), etc. Still further alternatively or additionally, in some embodiments, a VOC may be or comprise a component of a petroleum fuels, a hydraulic fluid, a paint thinner, a dry cleaning agent, etc. VOCs are common ground-water contaminants. In some embodiments, a VOC may be emitted (e.g., as a gas) from a solid or liquid such as, for example, a paint or lacquer, a paint stripper, cleaning supplies, pesticides, building materials or furnishings, office equipment such as copiers and printers, a correction fluid or carbonless copy paper, graphics and/or craft materials including glues and adhesives, permanent markers, photographic solutions, etc. In some embodiments, a VOC has a vapor pressure of about 0.01 kPa or more 20° C., or otherwise having a corresponding volatility under the particular conditions in which it is utilized and/or maintained.

### BRIEF DESCRIPTION OF THE DRAWING

FIG. 1 is a schematic of a typical leaf cross-section, shown are tissues of particular interest such as the cuticle, stoma, and intracellular space.

FIG. 2 is a schematic representation of certain enzymes, cofactors, and substrates related to formaldehyde capture and metabolism utilized herein.

FIG. 3 is a schematic representation of certain enzymes, cofactors, and substrates related to benzene, toluene, ethylbenzene, and xylene (BTEX) capture and metabolism utilized herein.

FIG. 4 is a map and reading frame expression analysis of an exemplary construct comprising formaldehyde metabolism enzymes.

FIG. 5 is a map of an exemplary plasmid construct containing a combination of transcriptional units comprising pollution metabolizing enzymes as described herein. This exemplary construct comprises: 1) two formaldehyde degrading enzymes FALDHEa and FDH3 linked with an IntF2A self-excising domain and a metabolically downstream HPS-Bm/PHI-Bm fusion protein; 2) an exemplary BTEX metabolizing enzyme, TodC1; 3) an exemplary stomatal density modulating protein, AtStomagen; 4) two optional enzymes that increase astaxanthin levels in leaves; and 5) an hpt gene encoding a hygromycin resistance marker. Gene of interest sequences are operably linked to various promoters, and followed by terminator sequences. Proteins can optionally be fused with a cellular localization signal.

FIG. 6 shows exemplary multiplex PCR genotyping results for ten successfully transformed Epipremnum aureum lines. Shown are transcriptional units coding for an exemplary formaldehyde degrading pathway: DASCanbo (Top band) and DAKY (Bottom band). Genotyping was performed using gene specific primers. The two last wells correspond to samples from wildtype (WT) non-transformed Epipremnum aureum acting as negative controls.

FIG. 7 shows exemplary qPCR results showing mRNA transcript levels of eight successfully transformed Epipremnum aureum lines that correctly express the FALDHEa gene. The two last entries correspond to samples of non-transformed plants as a negative control.

FIG. 8 is a representative fluorescence confocal microscopy image of a transformed Epipremnum aureum callus (pre-differentiation) expressing a formaldehyde metabolizing protein fused with a GFP tag.

FIG. 9 is a representative fluorescence confocal microscopy image of a developed Epipremnum aureum leaf expressing a formaldehyde metabolizing protein fused with a GFP tag.

FIG. 10 presents a graphical representation of bacterial growth (Mc8) when grown on increasing concentrations of formaldehyde. The X axis represents time, while the Y axis represents bacterial growth as measured by optical density at 600 nm.

FIG. 11A-B present a graphical representation of exemplary experiments measuring formaldehyde concentrations in growth media for WT MoCBMB20 bacteria (grey) when compared to an evolved strain FR4S (turquoise). FIG. 11A shows the removal of Formaldehyde (Y axis, measured in mM) from culture media over time (X axis, measured in hours). FIG. 11B shows the percentage of formaldehyde left in medium (Y axis) following culturing for a period of time with starting concentrations of formaldehyde ranging from 1 mM to 22 mM (X axis).

FIG. 12 presents a graphical representation of exemplary experiments measuring formaldehyde concentrations in growth media for WT MoCBMB20 bacteria (grey) when compared to an evolved strain (turquoise solid line), or a strain that has been selected for (turquoise dotted line). The Y axis represents formaldehyde concentrations in mM, while the X axis represents time in hours.

FIG. 13A-B presents a graphical representation of exemplary experiments measuring removal of atmospheric toluene by plant microbiome combinations. Wild type microbiomes are presented in grey, while evolved microbiomes are presented in turquoise. Atmospheric toluene levels are depicted on the Y axis (measured in PPM), while time is presented on the X axis (measured in hours), experiments were performed in a sealed 2 L chamber. FIG. 13A present a graphical representation of removal of atmospheric toluene by plant microbiome combinations during a 12 hour period. FIG. 13B present a graphical representation of removal of atmospheric toluene by plant microbiome combinations during a 60 hour period.

FIG. 14A-B presents a graphical representation of exemplary experiments measuring removal of atmospheric benzene by plant microbiome combinations. Wild type microbiomes are presented in grey, while evolved microbiomes are presented in turquoise. Atmospheric benzene levels are depicted on the Y axis (measured in PPM), while time is presented on the X axis (measured in hours), experiments were performed in a sealed 2 L chamber. FIG. 14A present a graphical representation of removal of atmospheric benzene by plant microbiome combinations during a 12 hour period. FIG. 14B present a graphical representation of removal of atmospheric benzene by plant microbiome combinations during a 60 hour period.

FIG. 15 presents a graphical representation of exemplary experiments measuring removal of atmospheric Xylene by plant microbiome combinations. Wild type microbiomes are presented in grey, while evolved microbiomes are presented in turquoise. Atmospheric Xylene levels are depicted on the Y axis (measured in PPM), while time is presented on the X axis (measured in hours), experiments were performed in a sealed 2 L chamber.

FIG. 16 shows formaldehyde bioremediation via Epipremnum aureum inoculation with Methylobacterium extorquens PA1 (MePA1) and Methylobacterium oryzae CBMB20 (MoCBM) and Pseudomonas putida F1 (PpF1).

FIG. 17A-D show toluene phytoremediation via Epipremnum aureum inoculation with the fungus Cladophialophora psammophila (Cp) or Cladophialophora immunda (Ci). FIG. 17A shows the phytoremediation capacity of the resulting plants measured at 24 h. FIG. 17B shows the phytoremediation capacity of the resulting plants measured at 1 week. FIG. 17C shows the phytoremediation capacity of the resulting plants measured at 2 weeks. FIG. 17D shows the phytoremediation capacity of the resulting plants measured at 4 weeks.

FIG. 18A-18B show formaldehyde phytoremediation capacity in transgenic plants via the xylulose monophosphate (XuMP) pathway. FIG. 18A shows the gaseous concentration of formaldehyde measured before and after exposure to high levels of formaldehyde for 24 hours exposure, the results are normalized by leaf surface area and the WT value is set at 100. FIG. 18B shows metabolomics results of trangenic plants exposed to 0 or 5 mM formaldehyde over 18 hours.

FIG. 19A-B show formaldehyde phytoremediation capacity in transgenic plants via the Serine pathway. FIG. 19A shows the gaseous concentration of formaldehyde measured before and after exposure to high levels of formaldehyde for 24 hours exposure, the results are normalized by leaf surface area and the WT value is set at 100. FIG. 19B shows metabolomics results of trangenic plants exposed to 0 or 10 mM formaldehyde over 18 hours.

FIG. 20 shows Benzene, Toluene, Ethylbenzene or Xylene (BTEX) phytoremediation capacity in transgenic plants after exposure to high levels of BTEX for 24 hours.

FIG. 21A-C show stomatal density and phytoremediation experimental in a model plant, Arabidopsis thaliana. FIG. 21A shows microscopy image of Arabidopsis thaliana leaf surface of a WT or transgenic plant overexpressing the gene, At_Caprice. FIG. 21B is a plot of the various independent Arabidopsis thaliana transgenic lines overexpressing At_Caprice stomatal density and amount of formaldehyde remediated by the plant. FIG. 21C shows formaldehyde phytoremediation capacity of WT Arabidopsis thaliana or At_Caprice, Os_Stomagen and At_Stomagen transgenic lines.

FIG. 22A-B shows the capacity of regulatory elements to increase expression levels of a polypeptide. FIG. 22A shows single cell fluorescence levels, reflecting promoter/terminator strengths in Epipremnum aureum leaf mesophyll cells. FIG. 22B shows a list of a subset of promoters and terminator identified in FIG. 22A.

### DETAILED DESCRIPTION OF CERTAIN EMBODIMENTS

### Indoor Air Quality

Indoor air contamination is a complex problem involving particles (such as dust and smoke), biological agents (e.g., microbial agents such as molds, spores, viruses), radon, asbestos, and gaseous contaminants such as CO, CO₂, NOₓ, SOₓ, aldehydes and VOCs (Volatile Organic Compounds). Among these, at least VOCs are strongly suspected to cause many Indoor Air Quality (IAQ) associated health problems and “sick-building” symptoms (see e.g., Wallace, 2001; Jones, 1999; Wieslander et al., 1997; Yu and Crump, 1998). In some embodiments, the present disclosure is directed to technologies designed to ameliorate the effects of indoor air contamination.

It is estimated that Americans spend nearly 90% of their time indoors, and that nearly 25% of US residents are affected by poor IAQ either at the workplace or at home. The US Environmental Protection Agency (EPA) ranks poor IAQ among its largest national environmental threats. Its counterpart, the European Environmental Agency (EEA) has described IAQ as one of the priority concerns for children's health, similar issues are faced worldwide (see e.g., Zhang and Smith, 2003; Observatory on Indoor Air Quality, 2006, Zumairi et al., 2006). In some cases, buildings can contain such high levels of contaminants that they are qualified as “sick” because exposure to them results in multiple sickness symptoms (e.g. headache, fatigue, skin and eye irritations, and/or respiratory illness). This condition is commonly described as “sick-building syndrome” (SBS) (see e.g., Burge, 2004).

It has been suggested that indoor air pollution causes between 65,000 and 150,000 deaths per year in the US, which is comparable to outdoors pollution induced mortality (see e.g., Lomborj, 2002). IAQ is also thought to impact work productivity, for example, Wargocki et al. (1999) showed subjects exposed to a typical indoor pollution source (e.g., plastic carpet) typed 6.5% less than control subjects. Likewise, certain other empirical studies have shown that the use of ventilation rates lower than 25 L s-1 per person in commercial and institutional buildings was correlated to an increase in the number of short-term sick leaves taken by employees (see e.g., Sundell, 2004). Using these data, at the turn of the century it was estimated that in the USA alone, $40-200 billion (USD) could be saved or gained in increased productivity annually by simply improving IAQ (in 1996 USD; Fisk, 2000). This estimate is thought to have increased as time has passed. In fact, by the early 2000s, this problem was already driving an important IAQ market that reached $5.6 billion in 2003 in the USA (Market report: indoor air quality, 2004).

Interestingly, there is no clear or unanimous public definition of what a VOC is. For example, the US EPA defines VOCs as substances with vapor pressure greater than 0.1 mmHg, while the Australian National Pollutant Inventory defines them as any chemical based on carbon chains or rings with a vapor pressure greater than 2 mm Hg at 25° C., and the EU defines them as chemicals with a vapor pressure greater than 0.074 mm Hg at 20° C. In addition, in some cases, chemicals such as CO, CO2, CH4, and sometimes aldehydes, are often excluded. Finally, additional sub-classifications such as Very Volatile Organic Compounds (VVOCs) or Semi Volatile Organic Compounds (SVOCs) have been used in the context of IAQ measurements (see e.g., Crump, 2001; Ayoko, 2004).

Several organizations such as the World Health Organization (WHO), the US EPA, or the OQAI (French Indoor Air Quality Observatory), have established lists of priority indoor air pollutants (see e.g., WHO, 2000; Johnston et al., 2002; Mosqueron and Nedellec, 2002, OQAI) based on the ubiquity, concentration, and potential toxic effect of the substances involved. These lists are relatively similar and systematically include aldehydes, aromatics, halogenates, and certain biocides. It is thought that certain differences in the classifications are likely due to the type of pollution taken into account, (only chemicals for the EPA, no mixtures such as tobacco smoke for the OQAI) and the geographic specificities of indoor air pollution. For example, geographically and/or culturally related variations in building materials, consumables such as cleaning products, and/or types of ventilation utilized can generate differences in measured indoor air pollutants and pollution levels (see e.g., Sakai et al., 2004). It is thought that various governing bodies IAQ priority lists will most likely evolve upon new analytical and toxicological findings. For example, as studies, data, and analytical methods improve, certain pollutants more relevant to important IAQ factors can be highlighted, e.g., the health effects of chronic exposure to multiple pollutants at low concentration (see e.g., Mosqueron and Nedellec, 2002). It is hypothesized that lack of relevant data and/or analysis explains why there are so few consistent guidelines for VOC indoor air concentrations currently available (see e.g., WHO, 2000; Canada, 1987).

In certain situations, hundreds of VOCs can be found simultaneously in indoor air, and that these compounds can exhibit very large variations in concentration as well as physical, chemical, and biological properties. Furthermore, while not being bound by current theory, it is thought that the composition of pollutants in a given enclosure can vary in time, e.g., the concentration of VOCs released from coating and furniture generally decreases in time, whereas the release of other certain substances depends on human activities or even respiration (see e.g., Ekberg, 1994; Phillips, 1997; Miekisch et al., 2004). While not being bound by current theory, it is thought that primary emissions of VOCs constitute a major source in new or renovated dwellings, particularly during the first few months following construction, whereas physical and chemical deterioration of buildings material (named secondary emission) later becomes a main mechanisms of VOC release (see e.g., Wolkoff and Nielsen, 2001; Yu and Crump, 1998). While not being bound by current theory, it is thought that indoor VOC concentrations can depend on the total space volume, pollutant production rate, pollutant removal rates, indoor-outdoor air exchange rates, and outdoor VOC concentrations (see e.g., Salthammer, 1997).

It is estimated that typical air exchange rates in rooms without mechanical ventilation systems can range from 0.1 h⁻¹ to 0.4 h⁻¹. In general, indoor VOC concentrations are higher than outdoor concentrations as VOCs are often released from human activities and a wide variety of materials such as floorings, linoleum, carpets, paints, surface coatings, furniture etc. (see e.g., Yu and Crump, 1998). For instance, Salthammer (1997) demonstrated that certain furniture coatings could release 150 different VOCs (mainly aliphatic and aromatic aldehydes, aromatic hydrocarbons, ketones, esters and glycols) at Total VOC (TVOC) concentrations up to 1288 μg m-3 in test chamber studies, and TVOC emission rates as high as 22,280 μg m-2 h-1 have been recorded from vinyl/pvc flooring (Yu and Crump, 1998). Additionally, certain molds and bacteria can contribute significantly to the presence of particles (spores) and VOCs in indoor pollution (see e.g., Schleibinger et al., 2004). It is thought that microbial development in buildings may provoke toxic and allergic responses, and can generally be found in places where humidity accumulates (e.g., areas with defective heating and air conditioning systems, garbage disposals, bathrooms, areas with water leaks, etc.). Thus, although in some situations, the individual concentrations of each contaminant may generally be considered as low (μg m-3), it is feasible for several hundred contaminants to be found simultaneously, resulting in significant TVOC levels. Indeed, Kostiainen (1995) demonstrated that individual concentrations of selected pollutants were 5-1000 times higher in 38 Finish sick-houses (defined as houses in which people experienced symptoms associated with SBS) than their mean concentrations in 50 normal houses used as reference, with over 200 VOCs being simultaneously detected in 26 of the houses investigated. This same study also reported a maximal TVOC concentration of 9538 μg m-3 in one sick house compared to the mean concentration of 121 μg m-3 recorded in normal houses. In line with these results, Brown and Crump (1996) recorded TVOC concentrations up to 11,401 μg m-3 in UK homes and Daisey et al. (1994) reported indoor TVOC concentrations of 230-700 μg m-3 (geometric mean of 510 μg m-3) in 12 Californian office buildings. While it is not simple to correlate TVOC concentration with health effects, (as this generic parameter does not reflect the individual differences in toxicities found among indoor air VOCs), it has been empirically reported that experiences of eye, nose, or mouth irritation is increased at 5000-25,000 μg TVOC m-3 (Andersson et al., 1997).

Although indoor VOCs such as benzene or some polycyclic aromatic hydrocarbons are recognized as human carcinogens, a direct association between exposure to VOCs and SBS symptoms or cancer has not been fully established at typical indoor air concentrations (Wallace, 2001). However, several studies have correlated exposure to low concentrations of these pollutants with increased risks of cancer, or eye and airways irritations (Vaughan et al., 1986, Wallace, 1991, Wolkoff and Nielsen, 2001). Certain symptoms such as headache, drowsiness, fatigue and confusion have been recorded in subjects exposed to 22 VOCs at 25 μg m-3 (Hudnell et al., 1992), while, exposure to 1000 μg m-3 of formaldehyde can cause coughing and eye irritation. In addition, many VOCs thought “harmless” may react with oxidants such as ozone, producing highly reactive compounds that can be more harmful than their precursors, some of which are sensory irritants (Sundell, 2004; Wolkoff et al., 1997; Wolkoff and Nielsen, 2001). Finally, it is hypothesized that reported concentrations of VOCs based on stationary measurement may lead to a systemic underestimation of real VOC exposure. For example, the real exposure of subjects evaluated in epidemiological studies may be 2-4 times higher than levels reported, as concentrations in breathing zones could be significantly higher than those recorded with traditional methods (Rodes et al., 1991; Wallace, 1991; Wolkoff and Nielsen, 2001). In certain embodiments, technologies described herein (e.g., compositions and methodologies) are designed to remove certain VOCs from the environment, increasing the quality of indoor air. In some embodiments, technologies described herein reduce symptoms associated with syndromes such as SBS. In certain embodiments, technologies described herein increase certain quality of life metrics.

In certain embodiments, technologies described herein are directed to the removal and/or remediation of certain volatile chemicals, such as formaldehyde, methanol, benzene, toluene, ethylbenzene, and/or xylene. In certain embodiments, technologies described herein are directed to the removal and/or remediation of formaldehyde. In certain embodiments, technologies described herein are directed to the removal and/or remediation of methanol. In certain embodiments, technologies described herein are directed to the removal and/or remediation of benzene. In certain embodiments, technologies described herein are directed to the removal and/or remediation of toluene. In certain embodiments, technologies described herein are directed to the removal and/or remediation of ethylbenzene. In certain embodiments, technologies described herein are directed to the removal and/or remediation of xylene.

### Formaldehyde

In some embodiments, technologies described herein are particularly amenable for the removal of aromatic formaldehyde. In some embodiments, formaldehyde metabolizing enzymes (e.g., as described herein) are introduced to a composition (e.g., as described herein, e.g., a plant and/or a microorganism) and facilitate the removal and/or remediation of formaldehyde. In certain embodiments, formaldehyde (HCHO) destined for removal and/or remediation by technologies described herein can be from numerous sources. For example, in certain embodiments, targeted HCHO is industrially produced from natural gas, and/or is produced from household products such as but not limited to adhesives, bonding agents, and/or solvents.

While not being bound by current theory, HCHO is thought to react as an electrophile with the side-chains of arginine and lysine and the amino groups of RNA and DNA, which in some cases causes protein-protein, protein-DNA, and/or DNA-DNA cross-links. In part based on these molecular characteristics, HCHO is suspected to be carcinogenic and a potentially causative agent in cases of sick-house syndrome. In addition, HCHO is also known as one of the major VOCs of air pollution and the WHO has established an air quality guideline of 0.1 mg m-3. The potential utilization of houseplants for the removal of VOCs was first proposed by Wolverton et al., 1984, while the authors found certain house plants appeared to have a relatively high capacity to remove HCHO from the air, later studies suggest that the primary organisms involved in HCHO removal from the air may not be the plants themselves, but rather microorganisms living symbiotically with the plants, e.g., members of the phyllosphere, rhizosphere, and/or endosphere.

### Methanol

In some embodiments, technologies described herein are particularly amenable for the removal of aromatic methanol. In certain embodiments, components of metabolic pathways suitable for the phytoremediation of formaldehyde may also be utilized for the phytoremediation of methanol. In some embodiments, methanol dehydrogenase (mdh) is introduced and facilitates the metabolism of methanol into formaldehyde. In some embodiments, technologies described herein suitable for phytoremediation of formaldehyde may also increase methanol metabolism. In some embodiments, such methanol metabolism may be the result of increased downstream flux e.g., increased metabolism of formaldehyde may result in increased metabolism of methanol.

### Benzene, Toluene, Ethylbenzene, and Xylene (BTEX)

In some embodiments, technologies (e.g., methods and/or compositions) provided herein are particularly amenable for the removal of benzene, toluene, ethylbenzene, and/or xylene (BTEX) from air.

In some embodiments, technologies provided herein are particularly amenable for the removal of aromatic benzene. In some embodiments, benzene metabolizing enzymes (e.g., as described herein) are introduced to a composition (e.g., as described herein, e.g., a plant and/or a microorganism) and facilitate the removal and/or remediation of benzene. Benzene is a chemical that is a colorless or light yellow liquid at room temperature, and it can be described as having a sweet odor. Benzene is highly flammable, and has the chemical formula C₆H₆, with a molecular mass of 78.11 g/mol. Benzene evaporates into the air very quickly, and its vapor is heavier than air, meaning it may sink into and accumulate in low-lying areas. Benzene dissolves only slightly in water and often will float on top of water. In some embodiments, benzene destined for removal and/or remediation by technologies described herein can be formed from natural processes and/or human activities. In certain embodiments, natural sources of benzene include volcanoes and fires. In certain embodiments, benzene is a product of crude oil, gasoline, and/or cigarette smoke. In some embodiments, benzene is produced industrially, e.g., benzene is widely used in the United States and ranks in the top 20 chemicals for production volume. In some embodiments, benzene is produced to make plastics, resins, nylon, and/or synthetic fibers. In some embodiments, benzene is also used to make some types of lubricants, rubbers, dyes, detergents, drugs, and/or pesticides. In certain embodiments, indoor air may contain higher levels of benzene than outdoor air. Without being bound by theory, it is thought that benzene in indoor air can come from products that contain benzene such as glues, paints, furniture wax, and detergents. Additionally, without being bound by theory, air around hazardous waste sites or gas stations can contain higher levels of benzene than in other areas. Finally, in certain embodiments, a source of indoor air benzene is smoke (e.g., tobacco smoke, coal smoke, wood smoke, incense, etc.). In some embodiments, benzene destined for removal and/or remediation by technologies described herein may be produced from, but is not limited to the sources described herein.

In some embodiments, technologies provided herein are particularly amenable for the removal of aromatic ethylbenzene. In some embodiments, ethylbenzene metabolizing enzymes (e.g., as described herein) are introduced to a composition (e.g., as described herein, e.g., a plant and/or a microorganism) and facilitate the removal and/or remediation of ethylbenzene. Ethylbenzene is used in the production of styrene, solvents, as a constituent of asphalt and naphtha, and in fuels. Ethylbenzene is a colorless liquid that can be described as smelling like gasoline. The chemical formula for ethylbenzene is C₈H₁₀, and the molecular weight is 106.16 g/mol. While not being bound by current theory, the EPA has classified ethylbenzene as a Group D chemical, (not classifiable as to human carcinogenicity) however, certain experiments have suggested that exposure to ethylbenzene in animal models by inhalation can result in a statistically significant increased incidence of kidney and testicular tumors in male rats, and a suggestive increase in kidney tumors in female rats, lung tumors in male mice, and liver tumors in female mice.

While not being bound by current theory, it is thought that acute high levels of aromatic benzene and/or ethylbenzene exposure may lead to the following signs and/or symptoms within minutes to several hours following exposure: drowsiness, dizziness, rapid or irregular heartbeat, headaches, tremors, confusion, unconsciousness, and/or death (at very high levels). While not being bound by current theory, it is thought that eating foods and/or drinking beverages containing high levels of benzene and/or ethylbenzene can cause the following symptoms within minutes to several hours following exposure: vomiting, irritation of the stomach, dizziness, sleepiness, convulsions, rapid or irregular heartbeat, and/or death (at very high levels). In some cases, if a person vomits because of swallowing foods or beverages containing benzene, the vomit could potentially be sucked into the lungs, resulting in breathing problems and/or coughing. While not being bound by current theory, it is thought that direct exposure of the eyes, skin, and/or lungs to benzene can cause tissue injury and/or irritation.

While not being bound by current theory, it is thought that blood is one of the tissues most effected from long term (e.g., exposure of a year or more) benzene and/or ethylbenzene exposure, for example, exposure can cause harmful effects to bone marrow and can cause a decrease in red blood cells, potentially leading to anemia. While not being bound by current theory, it is thought that benzene and/or ethylbenzene can also cause excessive bleeding and can affect the immune system, increasing the chance for infection. It has been reported that some women who breathed high levels of benzene for many months had irregular menstrual periods and a decrease in the size of their ovaries. It is not currently known whether benzene exposure affects the developing fetus in pregnant women or fertility in men. However, while not being bound by current theory, certain animal studies have shown low birth weights, delayed bone formation, and bone marrow damage when pregnant animals inhaled benzene. The United States Department of Health and Human Services (DHHS) has determined that benzene causes cancer in humans, particularly leukemia. In certain embodiments, technologies described herein may be utilized to decrease the incidence of certain diseases related to exposure to certain air pollutants (e.g., VOCs, e.g., formaldehyde, methanol, benzene, toluene, ethylbenzene, and/or xylene).

In some embodiments, technologies provided herein are particularly amenable for the removal of aromatic toluene. In some embodiments, toluene metabolizing enzymes (e.g., as described herein) are introduced to a composition (e.g., as described herein, e.g., a plant and/or a microorganism) and facilitate the removal and/or remediation of toluene. Toluene is a chemical that in liquid form is colorless, and is thought to have a sweet, pungent, benzene-like odor. Toluene is also known as methyl benzene, methyl benzol, phenyl methane, and/or toluol, and has a chemical formula of C₆H₅CH₃, with a molecular weight of 92.14 g/mol. Toluene occurs naturally in crude oil and in the tolu tree. In certain cases, toluene is produced in the process of making gasoline and other fuels from crude oil and in making coke from coal. In certain cases, toluene is used in making paints, paint thinners, fingernail polish, lacquers, adhesives, and rubber and in some printing and leather tanning processes. In certain cases, toluene is used in the production of benzene, nylon, plastics, and polyurethane and the synthesis of trinitrotoluene (TNT), benzoic acid, benzoyl chloride, and toluene diisocyanate. In certain cases, toluene is also added to gasoline along with benzene and xylene to improve octane ratings.

While not being bound by current theory, it is thought that acute high levels of toluene exposure may lead to the following signs and/or symptoms within minutes to several hours following exposure: eye and/or nose irritation, lassitude (weakness, exhaustion), confusion, euphoria, dizziness, headache, dilated pupils, lacrimation (discharge of tears), anxiety, muscle fatigue, insomnia, paresthesia, dermatitis, liver damage, and/or kidney damage.

In some embodiments, technologies provided herein are particularly amenable for the removal of aromatic xylene. In some embodiments, xylene metabolizing enzymes (e.g., as described herein) are introduced to a composition (e.g., as described herein, e.g., a plant and/or a microorganism) and facilitate the removal and/or remediation of xylene. Xylene is a colorless, flammable liquid and is thought to have a sweet odor. While not being bound by current theory, it is thought that there are three forms of xylene in which the methyl groups vary on the benzene ring: meta-xylene, ortho-xylene, and para-xylene (m-, o-, and p-xylene). In certain cases, xylene is also known as xylol or dimethylbenzene. In certain cases, xylene evaporates and burns easily. In certain cases, xylene does not mix well with water; however, it does mix with alcohol and many other chemicals.

It is thought that xylene is one of the top 30 chemicals produced in the United States in terms of volume. In certain cases, xylene is used as a solvent in the printing, rubber, and leather industries. Along with other solvents, xylene can also be widely used as a cleaning agent, a thinner for paint, and in varnishes. In certain cases, xylene is used as a material in chemical, plastics, and synthetic fiber industries and as an ingredient in the coating of fabrics and papers. In certain cases, isomers of xylene are used in the manufacture of certain polymers such as plastics. In certain cases, xylene is found in airplane fuel and gasoline.

While not being bound by current theory, it is thought that short-term exposure of people to high levels of xylene can cause irritation of the skin, eyes, nose, and/or throat; difficulty in breathing; impaired function of the lungs; delayed response to visual stimulus; impaired memory; stomach discomfort; and/or possible changes in the liver and/or kidneys. While not being bound by current theory, it is thought that both short- and long-term exposure to high concentrations of xylene can also cause a number of effects on the nervous system, such as headaches, lack of muscle coordination, dizziness, confusion, and/or changes in one's sense of balance. While not being bound by current theory, it is thought that exposure to very high levels of xylene for a short period of time can lead to death.

While not being bound by current theory, results of certain studies in animals indicate that large amounts of xylene can cause changes in the liver and harmful effects on the kidneys, lungs, heart, and/or nervous system. It is thought that short-term exposure to very high concentrations of xylene in animals causes muscular spasms, incoordination, hearing loss, changes in behavior, changes in organ weights, changes in enzyme activity, and/or potentially death. In certain cases, animals that were exposed to xylene on their skin had irritation and/or inflammation of the skin. In certain cases, it is thought that long-term exposure of animals to low concentrations of xylene can cause harmful effects on the kidney (with oral exposure) and/or on the nervous system (with inhalation exposure). Currently, both the International Agency for Research on Cancer (IARC) and EPA have found that there is insufficient information to determine whether or not xylene is carcinogenic and consider xylene not classifiable as to its human carcinogenicity.

### Indoor Ornamental Plants

Among other things, the present disclosure recognizes the potential usefulness of indoor ornamental plants in combating poor indoor air quality. In some embodiments, an indoor ornamental plant may also be referred to as a houseplant. In some embodiments, an indoor ornamental plant is engineered to more readily metabolize certain pollutants (e.g., formaldehyde, methanol, BTEX, etc.) when compared to a reference indoor ornamental plant. In some embodiments, engineered ornamental plants provided herein are particularly amenable for the removal of aromatic pollutants. In some embodiments, pollutant metabolizing enzymes (e.g., as described herein) are introduced to an ornamental house plant and facilitate the removal and/or remediation of pollutants from an indoor environment.

Epipremnum aureum, (Aka Pothos, Golden Pothos, or Devil's Ivy)

In certain embodiments, a composition and/or method described herein comprises an indoor ornamental house plant that is Epipremnum aureum. Epipremnum aureum is a species of flowering plant in the arum family Araceae, native to Mo'orea in the Society Islands of French Polynesia. The species is a popular houseplant in temperate regions, but has also become naturalized in tropical and sub-tropical forests worldwide, including northern Australia, Southeast Asia, South Asia, the Pacific Islands and the West Indies (where it has caused severe ecological damage in some cases). The plant has a multitude of common names including golden pothos, pothos, Ceylon creeper, hunter's robe, ivy arum, silver vine, Solomon Islands ivy, marble queen, devil's vine, devil's ivy, and taro vine.

In certain embodiments, Epipremnum aureum is particularly amenable as an indoor ornamental house plant as it is considered hardy, is often difficult to kill, and generally stays green even when kept in the dark. In certain embodiments, Epipremnum aureum is an evergreen vine growing to 20 m (66 ft) tall, with stems up to 4 cm (2 in) in diameter, climbing by means of aerial roots which adhere to surfaces. In certain embodiments, Epipremnum aureum leaves are alternate, heart-shaped, entire on juvenile plants, but irregularly pinnatifid on mature plants, up to 100 cm (39 in) long and 45 cm (18 in) broad; juvenile leaves may be smaller, typically under 20 cm (8 in) long. In certain embodiments, Epipremnum aureum rarely flowers without artificial hormone supplements, but when it does, the flowers are produced in a spathe up to 23 cm (9 in) long. In certain embodiments, pothos produces trailing stems when it climbs up trees and/or other structures, and these trailing stems can take root when they reach the ground and grow along it. In certain embodiments, leaves on trailing stems grow up to 10 cm (4 in) long and are reminiscent of the leaves seen on pothos when it is cultivated as a potted plant. In certain embodiments, pothos can be considered a popular houseplant with numerous cultivars selected for leaves with white, yellow, or light green variegation. In certain embodiments, pothos can be used in decorative displays in shopping centers, offices, and/or other public locations in part because it requires little care and is also attractively leafy. In certain tropical countries, pothos may be found in parks and gardens and tends to grow naturally. In certain embodiments, as an indoor plant, pothos can reach more than 2 m in height, particularly when given adequate support (e.g., a structure to climb), but as an indoor plant, pothos generally fails to develop adult-sized leaves. In certain embodiments, pothos can be considered a “shady” plant, and optimal growth conditions may be achieved by providing indirect light. In certain embodiments, pothos can tolerate an intense luminosity, but long periods of direct sunlight may burn leaves. In certain embodiments, pothos thrives in temperature to tropical temperatures between 17 and 30° C. (63 and 86° F.). In some embodiments, pothos only requires watering when the soil feels dry to the touch. In some embodiments, pothos tolerates and may be benefited by supplemental fertilizers and may grow rapidly in hydroponic culture. In some embodiments, pothos is sometimes used in aquariums, e.g., it may be placed on top of the aquarium and allowed to grow roots into the water, this may be beneficial to the plant and the aquarium as pothos may absorb soluble nitrates and use them for growth.

In some embodiments, pothos may be considered as toxic to cats and dogs due to the presence of insoluble raphides. In some embodiments, care should be taken to ensure that pothos is not consumed by pets. In some embodiments, symptoms of pothos consumption may include oral irritation, vomiting, and/or difficulty in swallowing. In some embodiments, potentially due to calcium oxalate within pothos, it may be considered mildly toxic to humans as well. In some embodiments, possible side effects from consumption of E. aureum are atopic dermatitis (eczema) as well as burning and/or swelling of the region inside of and surrounding the mouth. In some embodiments, excessive contact with pothos may also lead to general skin irritation

### Alternative Ornamental Plants

One skilled in the art will recognize that many Ornamental Plants (e.g., indoor ornamental plants) are amenable to the methods described herein, and may provide substrates for the creation of useful compositions.

In certain embodiments, technologies described herein comprise an engineered indoor ornamental house plant that is of the family Araceae. In certain embodiments, an engineered indoor ornamental house plant can be a member of a genus such as but not limited to the genera Aglaonema, Alocasia, Amorphophallus, Anthurium, Caladium, Colocasia, Dieffenbachia, Epipremnum, Monstera, Philodendron, Rhaphidophora, Scindapsus, Spathiphyllum, Syngonium, Xanthosoma, Zamioculcas, and Zantedeschia. In some particular embodiments, an engineered indoor ornamental house plant may be a member of a species such as but not limited to Alocasia amazonica, Alocasia odora, Alocasia wentii, Alocasia zebrine, Dieffenbachia seguine, Philodendron cordatum, Monstera adansonii, Monstera deliciosa, Philodendron florida, Philodendron hederaceum, Philodendron Xanadu, Monstera obliqua, Syngonium podophyllum, and Zamioculcas zamiifolia.

In certain embodiments, technologies described herein comprise an engineered indoor ornamental house plant that is of the class Polypodiopsida (e.g., a fern). In some embodiments, an engineered indoor ornamental house plant can be a member of a genus such as but not limited to the genera Adiantum, Aglaomorpha, Asplenium, Blechnum, Cyathea, Davallia, Didymochlaena, Dryopteris, Humata, Microsorum, Nephrolepsis, Pellaea, Phlebodium, Platycerium, Polypodium, and Pteris. In certain embodiments, an engineered indoor ornamental house plant can be a member of a species such as but not limited to the species Adiantum hispidulum, Adiantum raddianum, Adiantum tenerum, Aglaomorpha coronans, Asplenium antiquum, Asplenium nidus, Blechnum gibbum, Cyathea cooperi, Davallia fejeensis, Didymochlaena truncatula, Dryopteris erythrosora, Humata tyermanii, Microsorum diversifolium, Nephrolepis cordifolia, Nephrolepis exaltata, Pellaea rotundifolia, Phlebodium aureum mandaianum, Platycerium bifurcatum, Polypodium formosanum, Pteris cretica, Pteris ensiformis, and Pteris quadriaurita,

In certain embodiments, technologies described herein comprise an indoor ornamental house plant that is a member of the family Marantaceae (e.g., of the genus Calatheas). In certain embodiments, an engineered indoor ornamental house plant can be a member of a species such as but not limited to the species Calathea ornata, Calathea rufibarba, Calathea orbifolia, Calathea roseopicta, Calathea zebrine, Calathea lancifolia, Calathea warscewiczii, Calathea louisae, Calathea veitchiana, Calathea picturata, Calathea ecuadoriana, Calathea gandersii, Calathea curaraya, Calathea libbyana, Calathea hagbergii, Calathea roseobracteata, Calathea paucifolia, Calathea ischnosiphonoides, Calathea multicinta, Calathea latrinotecta, Calathea dodsonii, Calathea anulque, Calathea lanicaulis, Calathea petersenii, Calathea pluriplicata, Calathea plurispicata, Calathea pallidicosta, Calathea congesta, and Calathea utilis.

In certain embodiments, technologies describe herein comprise and/or utilize an indoor ornamental plant that is a member of the family Asparagaceae (e.g., of the genus Dracaena or of the genus Beaucarnea. In certain embodiments, an engineered indoor ornamental house plant can be a member of a species such as but not limited to the species Dracaena angolensis, Dracaena marginata, Dracaena trifasciata,

In certain embodiments, technologies describe herein comprise and/or utilize an indoor ornamental plant that is a member of the family Bambusoideae (e.g., of the genus Phyllostachys). In certain embodiments, an engineered indoor ornamental house plant can be a member of a species such as but not limited to the species Phyllostachys aurea.

In certain embodiments, technologies describe herein comprise and/or utilize an indoor ornamental plant that is a member of the family Urticaceae (e.g., of the genus Pilea). In certain embodiments, an engineered indoor ornamental house plant can be a member of a species such as but not limited to the species Pilea peperomioides, Pilea cadierei, Pilea grandifolia, Pilea involucrata, Pilea microphylla, Pilea nummulariifolia, Pilea peperomioides

In certain embodiments, technologies describe herein comprise and/or utilize an indoor ornamental plant that is a member of the family Moraceae (e.g., of the genus Ficus). In certain embodiments, an engineered indoor ornamental house plant can be a member of a species such as but not limited to the species Ficus lyrata, Ficus altissima, Ficus elastica

In certain embodiments, technologies describe herein comprise and/or utilize an indoor ornamental plant that is a member of the family Araliaceae (e.g., of the genus Heptapleurum). In certain embodiments, an engineered indoor ornamental house plant can be a member of a species such as but not limited to the species Schefflera arboricola.

In certain embodiments, technologies describe herein comprise and/or utilize an indoor ornamental plant that is a member of the family Acanthaceae (e.g., of the genus Aphelandra). In certain embodiments, an engineered indoor ornamental house plant can be a n: member of a species such as but not limited to the species Aphelandra squamosal, Aphelandra squarrosa.

In certain embodiments, technologies describe herein comprise and/or utilize an indoor ornamental plant that is a member of the family Arecaceae (e.g., of the genus Howea or of the genus Dypsis). In certain embodiments, an engineered indoor ornamental house plant can be a member of a species such as but not limited to the species Dypsis lutescens, Howea forsteriana, Howea belmoreana.

In certain embodiments, technologies describe herein comprise and/or utilize an indoor ornamental plant that is a member of the family Strelitziaceae (e.g., of the genus Strelitzia). In certain embodiments, an engineered indoor ornamental house plant can be a member of a species such as but not limited to the species Strelitzia nicolai, Strelitzia reginae.

In certain embodiments, technologies describe herein comprise and/or utilize an indoor ornamental plant that is a member of the family (e.g., of the genus). In certain embodiments, an engineered indoor ornamental house plant can be a member of a species such as but not limited to the species

Engineering Ornamental Plants and/or Microbes

In some embodiments, the present disclosure provides technologies that comprise and/or utilize engineered ornamental plants and/or microbes including, for example, chemically engineered, environmentally engineered, and/or genetically engineered plants and/or microbes.

In some embodiments, chemical engineering may be or comprise exposure to one or more particular chemical agents (e.g., nutrients, mutagens, etc).

In some embodiments, environmental engineering may be or comprise exposure, maintenance, and/or cultivation under a specified set of conditions (e.g., light, temperature, pressure, pH, etc) and/or involving one or more particular manipulations (e.g., grafting, traditional cloning, re-potting, etc).

In some embodiments, genetic engineering may be or comprise introducing one or more genetic modifications (e.g., insertions, deletions, and/or alterations of one or more particular sequences—e.g., genes). In some embodiments, genetic modification may involve and/or be accomplished through performance of one or more of transformation, transduction, and/or other introduction of a transgene or other heterologous nucleic acid sequence; disruption and/or interference with expression of one or more genetic sequences (e.g., gene knockout, gene knockdown, etc), induction and/or amplification of expression of one or more genetic sequences, alteration (e.g., by mutagenesis such as targeted or random mutagenesis), etc. In some embodiments, genetic engineering may involve one or more of selective breeding, and/or directed evolution.

In some embodiments, a plant and/or microbe is genetically engineered through a process of selective breeding and/or directed evolution across multiple generations using at least one sufficiently selective pressure, followed by optional mutation identification (e.g., genotyping), and phenotypic analysis.

In some embodiments, a plant and/or microbe is genetically engineered through a process of random mutagenesis followed by screening for a trait of interest, optional mutation identification (e.g., genotyping), and phenotypic analysis.

In some embodiments, a plant and/or microbe is genetically engineered through a process of directed mutagenesis, followed by optional mutation verification (e.g., genotyping), and phenotypic analysis.

In some embodiments, a plant and/or microbe is genetically engineered through a process of transgene introduction, followed by optional mutation verification (e.g., genotyping), and phenotypic analysis.

In some embodiments, a plant and/or microbe is genetically engineered by introduction of a vector into such plant and/or microbe (e.g., into a cell or spore thereof). In some embodiments, a vector suitable for plant transformation is generated, is optionally verified through any appropriate technology (e.g., sequencing, PCR, gel electrophoresis), and is then inserted into a plant genome. In some embodiments, insertion into a plant genome can be accomplished through 1) Agrobacterium tumefaciens mediated gene insertion, or 2) biolistic mediated gene insertion (DNA bombardment method).

In some embodiments, A. tumefaciens insertion may be an appropriate methodology to use when a working protocol exists. In some embodiments, insertion of a gene into a plant comprises: 1) Agrobacterium transformation by electroporation, 2) selection of viable clones, and 3) plant infection; in some embodiments this process can allow for relatively high transformation efficiencies. In some embodiments, binary plasmids are utilized. In some embodiments, binary plasmids are compatible with A. tumefaciens-based transformations. In some embodiments, binary plasmids are utilized as part of a golden gate DNA assembly system.

In some embodiments, a biolistic particle delivery system, or “gene gun” approach is utilized to mediate gene insertion into a plant. In some embodiments, such an approach utilizes DNA-coated gold particles to deliver a vector of interest to cells, integrating all or at least a portion of the vector (e.g., a coding construct) inside a plants genome (e.g., any endogenous store of genetic material, e.g., DNA of the mitochondria, chloroplast, and/or nucleus). In some embodiments, such an approach creates an artificial chromosome. In some embodiments, an artificial chromosome is stably inherited through multiple generations. In some embodiments, a biolistic particle delivery system is utilized when no efficient A. tumefaciens mediated transformation protocol is available for a particular target species of plant. In some embodiments, a biolistic approach is preferential to A. tumefaciens-based transformations due to an inherent ability of biolistic introduction to target not only nuclear DNA, but also mitochondrial and/or chloroplastic DNA. In certain embodiments, a biolistic approach may be preferential due to an inherent ability to insert lower copy numbers (e.g., 1 copy), potentially reducing the odds of transgene silencing by endogenous defense mechanisms.

### Modifying Endogenous Gene and Transgene Expression

The present disclosure recognizes that certain endogenous pathways found in plants may contribute to transgene silencing. To overcome said silencing, in certain embodiments, endogenous genes may be silenced (e.g., silenced, knocked out, knocked down, mutated, rendered impotent, etc.) to provide an in-vivo environment more amenable to transgene expression.

In some embodiments, exogenous transgenes inserted inside a plant are identified and silenced by a plant's endogenous gene regulation machinery. In certain embodiments, such a scenario increases in likelihood as additional transgenes are inserted into one organism. In some embodiments, certain approaches are utilized that facilitate avoidance of transgene silencing, such approaches comprise but are not limited to: 1) utilizing different promoters for each transgene, 2) inserting introns in a gene of interest, 3) utilizing codon optimization to increase transgene translational efficiencies, and/or 4) including multiple functional translational products in one highly heterogeneous vector.

Random and/or Directed Mutagenesis of Plants and/or Microorganisms

Among other things, in some embodiments, the present disclosure provides compositions and methods suitable for engineering plants and/or microbes (e.g., potential microbiome components) with enhanced desirable characteristics through the use of random and/or directed mutagenesis, followed by selection, and phenotypic analysis.

In certain embodiments, random mutagenesis is mediated through exposure to radiation (e.g., X-rays, gamma radiation, UV radiation etc.), and/or exposure to a chemical mutagen (e.g., NaN₃, EMS, MNU etc.). Those skilled in the art are aware of the standard techniques used to randomly mutate plants and/or microbes.

In certain embodiments, following random mutagenesis, plants and/or microbes are screened for enhanced desirable characteristics (e.g., higher tolerance to and/or biodegradation rates of certain pollutants, e.g., VOCs, and/or e.g., an ability to grow on certain pollutants as a sole carbon source). In certain embodiments, plants and/or microbes with desirable characteristics are identified, isolated, and bred with other plants and/or microbes with desirable characteristics. In some embodiments, a multi-generational program is initiated and desirable traits are enhanced through successive generations.

In certain embodiments, characteristics, enhanced or otherwise, of one plant and/or microbe may be transfer to another through horizontal gene transfer. For example, in certain embodiments, horizontal gene transfer may comprise transfer of a desired trait (e.g., high biodegradation rate of a certain pollutant), from one host organism to another acceptor organism (e.g., from one or more microorganisms into one or more other microorganisms). In certain embodiments, an acceptor organism may also comprise an additional trait of interest, (e.g., one or more desirable traits, e.g., one or more genes contributing to biodegradation of another and/or the same pollutant, and/or another desirable trait such as stable interaction and/or survival in the plant-soil-pot system).

Selective Breeding of Plants and/or Microorganisms

Among other things, the present disclosure provides compositions and methods suitable for engineering plants and/or microbes (e.g., potential microbiome components) with enhanced desirable characteristics.

In certain embodiments, wild type and/or naturally occurring plants and/or microbes are screened for desirable characteristics (e.g., higher tolerance to and/or biodegradation rates of certain pollutants, e.g., VOCs). In certain embodiments, plants and/or microbes with desirable characteristics are identified, isolated, and bred with other plants and/or microbes with desirable characteristics. In some embodiments, a multi-generational program is initiated and desirable traits are enhanced through successive generations.

Directed Evolution of Plants and/or Microorganisms

Among other things, the present disclosure provides compositions and methods suitable for engineering microbes (e.g., potential microbiome components) with enhanced desirable characteristics.

In certain case studies comprising tested plants, it is thought that potentially up to a third of the phytoremediation of indoor air pollutants is due to microbiome components. In some cases, species of bacteria and/or fungi living on and/or around a plant stem and/or leaves (phyllosphere), roots (rhizosphere), and/or within the plant (endosphere) are numerous and may be plant-specific. It is thought that some microbiome components, such as Methylobacterium and Pseudomonas putida, are naturally capable of absorbing and metabolizing pollutants such as formaldehyde and BTEX respectively. In some embodiments of technologies described herein (e.g., of compositions and/or methods), once a particular microbe is identified and optionally isolated (e.g., through monoculture), such a microbe (e.g., bacteria, fungi, etc.) are subjected to an artificial selective pressure over multiple generations, facilitating directed evolution, and an enhancement of certain desirable characteristics (e.g., improvements to their plant symbiosis and/or their phytoremediation capabilities). In some embodiments of technologies described herein, after directed evolution, a microbe may be utilized alone, or may be inoculated into and/or onto a plant and therefore contribute to overall phytoremediation (e.g., adsorption and/or degradation of VOCs).

### Transgenic Vectors

In certain embodiments, the present disclosure provides vectors suitable for engineering of plants and/or microbes. In certain embodiments, the present disclosure provides polynucleotide vectors suitable for transgene introduction into plants and/or microbes. In certain embodiments, polynucleotide vectors comprise a coding sequence and may be referred to herein as a construct. In some embodiments, a coding sequence may comprise the genetic information required to create useful products, e.g., RNA and/or proteins that may confer desirable traits (e.g., higher tolerance to and/or biodegradation rates of certain pollutants, e.g., VOCs).

In some embodiments, a vector described herein can further include regulatory and/or control sequences that alter the transcription and/or translation of an encoded gene, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some embodiments, a promoter can be a native promoter, a constitutive promoter, an inducible promoter, and/or a tissue-specific promoter. Non-limiting examples of transcriptional and/or translational control sequences are described herein.

### Exemplary Vector Components

### Cloning Vectors

In some embodiments, technologies described herein comprise a vector. In some embodiments, a vector is a transgenic vector. In some embodiments, a transgenic vector comprises a cloning vector. In certain embodiments, a transgenic vector comprises an engineered polynucleotide suitable for introduction into an organism.

In some embodiments, a transgenic vector may comprise a backbone sequence. In some embodiments, a transgenic vector may comprise at least one promoter. In some embodiments, a transgenic vector may comprise at least one 5′ UTR. In some embodiments, a transgenic vector may comprise at least one organelle localization signal. In some embodiments, a transgenic vector may comprise at least one gene of interest (e.g., an enzyme and/or protein of interest). In some embodiments, a transgenic vector may comprise at least one tag sequence (e.g., a fluorescent tag). In some embodiments, a transgenic vector may comprise at least one 3′ UTR. In some embodiments, a transgenic vector may comprise at least one transcription termination sequence. In some embodiments, a transgenic vector may comprise at least one selectable marker.

In some embodiments, the present disclosure provides compositions and methods suitable for engineering polynucleotide vectors (e.g., plasmids etc.). In certain embodiments, a polynucleotide vector comprises at least one transgene to be inserted into a plant and/or microbes genome (e.g., any store of genetic information, e.g., nuclear DNA, mitochondrial DNA, chloroplastic DNA etc.). One skilled in the art will recognize that in some embodiments, many molecular biology methodologies now exist that may facilitate engineering of vectors suitable for transgenic engineering. For example, in some embodiments, a method suitable for transgenic engineering may comprise the use of golden gate DNA assembly systems. In some embodiments, golden gate DNA assembly systems may be particularly amenable for creation of compositions described herein. In some embodiments, a transgenic engineering system comprises a three step hierarchical modular cloning scheme. In some embodiments, a golden gate DNA assembly system facilitates high efficiency assembly of complex multigene vectors that can encode entire pathways. In some embodiments, multigene vectors may begin as libraries of basic modules containing regulatory and/or coding sequences. In certain embodiments, a cloning process utilizes type IIS restriction enzymes. In some embodiments, transgenic engineering (e.g., for metabolic engineering) can be rendered highly efficient through use of golden gate DNA assembly systems as the inherent modularity facilitates iterative design, and building of multiple variants of a particular genetic circuit. In some embodiments, expression ratios of several genes can be obtained, and optimal parameters for a synthetic pathway can be engineered and tested in parallel. In certain embodiments, use of restriction enzymes during golden gate DNA assembly allows for high throughput engineering. In certain embodiments, use of restriction enzymes during golden gate DNA assembly allows for error-free engineering. In certain embodiments, use of restriction enzymes during golden gate DNA assembly allows for both high throughput and error-free engineering, which can be considered highly advantageous over traditional PCR-based cloning techniques. One skilled in the art will recognize that multiple DNA assembly and/or cloning technologies exist and may be suitable for the creation of vectors, and/or compositions described herein.

In certain embodiments, metabolic pathways described herein (e.g., pathways suitable for transgenic engineering, e.g., metabolic engineering) are tested in parallel, e.g., by simultaneously launching transformation of dozens of plant lines each with at least one DNA vector. In certain embodiments, metabolic pathways described herein (e.g., pathways suitable for transgenic engineering, e.g., metabolic engineering) are tested in parallel, e.g., by simultaneously launching the transformation of dozens of plant lines each with at least one different DNA vector. In some embodiments, compositions and methods describe herein are tested using a protoplasts system (e.g., a cell suspension). In some embodiments, use of golden gate DNA assembly and/or protoplast systems permits in vivo testing prior to plant transformation.

In some embodiments, a vector for metabolic engineering as described herein can be or comprise but is not limited to, a plasmid, a transposon, a cosmid, an artificial chromosome (e.g., a human artificial chromosome (HAC), a yeast artificial chromosome (YAC), a bacterial artificial chromosome (BAC), a P1-derived artificial chromosome (PAC)), a viral vector, a Gateway® plasmid, etc. In some embodiments, suitable vectors provided herein can be of different sizes.

In some embodiments, a vector is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, up to about 15 kb, up to about 16 kb, up to about 17 kb, up to about 18 kb, up to about 19 kb, up to about 20 kb, up to about 21 kb, up to about 22 kb, up to about 23 kb, up to about 24 kb, up to about 25 kb, up to about 26 kb, up to about 27 kb, up to about 28 kb, up to about 29 kb, up to about 30 kb, up to about 31 kb, up to about 32 kb, up to about 33 kb, up to about 34 kb, or up to about 35 kb. In some embodiments, a vector is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, about 1 kb to about 15 kb, 1 kb to about 16 kb, about 1 kb to about 17 kb, about 1 kb to about 18 kb, about 1 kb to about 19 kb, about 1 kb to about 20 kb, about 1 kb to about 21 kb, about 1 kb to about 22 kb, about 1 kb to about 23 kb, about 1 kb to about 24 kb, about 1 kb to about 25 kb, about 1 kb to about 26 kb, about 1 kb to about 27 kb, about 1 kb to about 28 kb, about 1 kb to about 29 kb, about 1 kb to about 30 kb, about 2 kb to about 12 kb, about 2 kb to about 14 kb, about 2 kb to about 16 kb, about 2 kb to about 18 kb, about 2 kb to about 20 kb, about 2 kb to about 22 kb, about 2 kb to about 24 kb, about 2 kb to about 26 kb, about 2 kb to about 28 kb, about 2 kb to about 30 kb, about 5 kb to about 10 kb, about 5 kb to about 12 kb, about 5 kb to about 14 kb, about 5 kb to about 16 kb, about 5 kb to about 18 kb, about 5 kb to about 20 kb, about 5 kb to about 22 kb, about 5 kb to about 24 kb, about 5 kb to about 26 kb, about 5 kb to about 28 kb, about 5 kb to about 30 kb, about 5 kb to about 32 kb, about 5 kb to about 34 kb, about 5 kb to about 36 kb, about 10 kb to about 12 kb, about 10 kb to about 14 kb, about 10 kb to about 16 kb, about 10 kb to about 18 kb, about 10 kb to about 20 kb, about 10 kb to about 22 kb, about 10 kb to about 24 kb, about 10 kb to about 26 kb, about 10 kb to about 28 kb, about 10 kb to about 30 kb, about 14 kb to about 16 kb, about 14 kb to about 18 kb, about 14 kb to about 20 kb, about 14 kb to about 22 kb, about 14 kb to about 24 kb, about 14 kb to about 26 kb, about 14 kb to about 28 kb, about 14 kb to about 30 kb, about 18 kb to about 20 kb, about 18 kb to about 22 kb, about 18 kb to about 24 kb, about 18 kb to about 26 kb, about 18 kb to about 28 kb, about 14 kb to about 30 kb, about 14 kb to about 32 kb, about 16 kb to about 34 kb, about 18 kb to about 36 kb, about 20 kb to about 22 kb, about 20 kb to about 24 kb, about 20 kb to about 26 kb, about 20 kb to about 28 kb, about 20 kb to about 30 kb, about 20 kb to about 32 kb, about 20 kb to about 34 kb, about 20 kb to about 36 kb, about 26 kb to about 30 kb, about 28 kb to about 30 kb, about 24 to about 26 kb, or about 25 to about 27 kb.

In some embodiments, a vector is an artificial chromosome and can include a total length of up to about 3000 kb, up to about 2900 kb, up to about 2800 kb, up to about 2700 kb, up to about 2600 kb, up to about 2500 kb, up to about 2400 kb, up to about 2300 kb, up to about 2200 kb, up to about 2100 kb, up to about 2000 kb, up to about 1900 kb, up to about 1800 kb, up to about 1700 kb, up to about 1600 kb, up to about 1500 kb, up to about 1400 kb, up to about 1300 kb, up to about 1200 kb, up to about 1100 kb, up to about 1000 kb, up to about 900 kb, up to about 800 kb, up to about 700 kb, up to about 600 kb, up to about 500 kb, up to about 400 kb, up to about 375 kb, up to about 350 kb, up to about 325 kb, up to about 300 kb, up to about 275 kb, up to about 250 kb, up to about 225 kb, up to about 200 kb, up to about 175 kb, up to about 150 kb, or up to about 125 kb.

In some embodiments, a vector is a viral vector and can have a total number of nucleotides of up to 10 kb. In some embodiments, a viral vector can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, about 1 kb to about 15 kb, about 1 kb to about 16 kb, about 1 kb to about 17 kb, about 1 kb to about 18 kb, about 1 kb to about 19 kb, about 1 kb to about 20 kb, about 1 kb to about 21 kb, about 1 kb to about 22 kb, about 1 kb to about 23 kb, about 1 kb to about 24 kb, about 1 kb to about 25 kb, about 1 kb to about 26 kb, about 1 kb to about 27 kb, about 1 kb to about 28 kb, about 1 kb to about 29 kb, or about 1 kb to about 30 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 2 kb to about 12 kb, about 2 kb to about 14 kb, about 2 kb to about 16 kb, about 2 kb to about 18 kb, about 2 kb to about 20 kb, about 2 kb to about 22 kb, about 2 kb to about 24 kb, about 2 kb to about 26 kb, about 2 kb to about 28 kb, about 2 kb to about 30 kb, about 5 kb to about 10 kb, about 5 kb to about 12 kb, about 5 kb to about 14 kb, about 5 kb to about 16 kb, about 5 kb to about 18 kb, about 5 kb to about 20 kb, about 5 kb to about 22 kb, about 5 kb to about 24 kb, about 5 kb to about 26 kb, about 5 kb to about 28 kb, about 5 kb to about 30 kb, about 10 kb to about 12 kb, about 10 kb to about 14 kb, about 10 kb to about 16 kb, about 10 kb to about 18 kb, about 10 kb to about 20 kb, about 10 kb to about 22 kb, about 10 kb to about 24 kb, about 10 kb to about 26 kb, about 10 kb to about 28 kb, about 10 kb to about 30 kb, about 14 kb to about 16 kb, about 14 kb to about 18 kb, about 14 kb to about 20 kb, about 14 kb to about 22 kb, about 14 kb to about 24 kb, about 14 kb to about 26 kb, about 14 kb to about 28 kb, about 14 kb to about 30 kb, about 18 kb to about 20 kb, about 18 kb to about 22 kb, about 18 kb to about 24 kb, about 18 kb to about 26 kb, about 18 kb to about 28 kb, about 14 kb to about 30 kb, about 20 kb to about 22 kb, about 20 kb to about 24 kb, about 26 kb to about 30 kb, about 28 kb to about 30 kb, or about 24 to about 26 kb.

### Promoters

In some embodiments, a vector comprises a promoter. The term “promoter” refers to a DNA sequence recognized by enzymes/proteins that can promote and/or initiate transcription of an operably linked gene. For example, a promoter typically refers to a nucleotide sequence to which an RNA polymerase and/or any associated factor binds and from which the process of and/or initiate of transcription can occur. Thus, in some embodiments, a vector comprises one of the non-limiting example promoters described herein operably linked to a coding region.

In some embodiments, a promoter is an inducible promoter, a constitutive promoter, a plant cell promoter, a viral promoter, a chimeric promoter, an engineered promoter, a tissue-specific promoter, or any other type of promoter known in the art.

In some embodiments, a promoter may comprise an additional regulatory region such as an enhancer and/or a 5′ UTR. In some embodiments, a promoter may be but is not limited to: 2× CaMV 35S, 2× CaMV 35S+5′UTR TMV, AtAct2, AtSUC2, H4, H4 (S. lycopersicum)+5′UTR, LHB1B1, LHB1B1 (A. thaliana)+5′UTR, Nos, Nos+5′UTR TMV, ocs, ocs (A. tumefaciens)+5′UTR, OsActin+5′UTR, PvUbi1+3, PvUbi1+3 promoter, PvUbi2, PvUbi2_mut, RbcS2B, RolC, rrEaActBlast2, rrEaAs2Blast1, rrEaDPA4Blast1, rrEaH3Blast2, rrEaUbiBlast1, RsS1, RTBV, ZmUbi, or any combination thereof.

In some embodiments, a promoter is one listed herein as set forth in any one of SEQ ID NOs: 1-48. In some embodiments, a promoter sequence is at least 85%, 90%, 95%, 98% or 99% identical to a promoter sequence represented by any one of SEQ ID NOs: 1-48. In some embodiments, a promoter is a characteristic portion of any one of SEQ ID NOs: 1-48.

The term “constitutive” promoter refers to a nucleotide sequence that, when operably linked with a nucleic acid encoding a protein (e.g., a metabolic protein), causes RNA to be transcribed from the nucleic acid in a cell under most or all physiological conditions. In certain embodiments, a suitable plant specific constitutive promoter may comprise but is not limited to: a Zea mays Ubiquitin 1 promoter (ZmUbi), an Oryza sativa Actin 1 promoter (OsAc1), a Panicum virgatum L. Ubiquitin 2 promoter (PvUbi2), a Panicum virgatum L. Ubiquitin 1 fusion promoter (PvUbi1+3), an Oryza sativa Cytochrome c gene promoter (OsCc1), an Epipremnum aureum Ubiquitin promoter (rrEaUbil or P1), an Epipremnum aureum Actin promoter, an Epipremnum aureum Histone H₃ promoter (rrEaH32 or P7), a Cauliflower Mosaic virus promoter (2× CaMV35S), a Agrobacterium tumefaciens Nopaline synthase gene promoter (NOS), an Epipremnum aureum ribulose bisphosphate carboxylase/oxygenase activase 2 (rrEaLeaf2) promoter, an Epipremnum aureum Metallothionein-like protein type 3 promoter (rrEaLeaflor P18), an Epipremnum aureum abscisic stress-ripening protein 2-like promoter (rrEaCons3 or P16), an Epipremnum aureum RNA-binding protein cabeza-like promoter (rrEaCons4), or a combination of any characteristic portion of any one or more of these promoters.

-Exemplary Zea mays Ubiquitin 1 promoter (ZmUbi1)
SEQ ID NO: 1
CTGCAGTGCAGCGTGACCCGGTCGTGCCCCTCTCTAGAGATAATGAGCATTGCATGTCTAAGTT
ATAAAAAATTACCACATATTTTTTTTGTCACACTTGTTTGAAGTGCAGTTTATCTATCTTTATA
CATATATTTAAACTTTACTCTACGAATAATATAATCTATAGTACTACAATAATATCAGTGTTTT
AGAGAATCATATAAATGAACAGTTAGACATGGTCTAAAGGACAATTGAGTATTTTGACAACAGG
ACTCTACAGTTTTATCTTTTTAGTGTGCATGTGTTCTCCTTTTTTTTTGCAAATAGCTTCACCT
ATATAATACTTCATCCATTTTATTAGTACATCCATTTAGGGTTTAGGGTTAATGGTTTTTATAG
ACTAATTTTTTTAGTACATCTATTTTATTCTATTTTAGCCTCTAAATTAAGAAAACTAAAACTC
TATTTTAGTTTTTTTATTTAATAATTTAGATATAAAATAGAATAAAATAAAGTGACTAAAAATT
AAACAAATACCCTTTAAGAAATTAAAAAAACTAAGGAAACATTTTTCTTGTTTCGAGTAGATAA
TGCCAGCCTGTTAAACGCCGTCGACGAGTCTAACGGACACCAACCAGCGAACCAGCAGCGTCGC
GTCGGGCCAAGCGAAGCAGACGGCACGGCATCTCTGTCGCTGCCTCTGGACCCCTCTCGAGAGT
TCCGCTCCACCGTTGGACTTGCTCCGCTGTCGGCATCCAGAAATTGCGTGGCGGAGCGGCAGAC
GTGAGCCGGCACGGCAGGCGGCCTCCTCCTCCTCTCACGGCACCGGCAGCTACGGGGGATTCCT
TTCCCACCGCTCCTTCGCTTTCCCTTCCTCGCCCGCCGTAATAAATAGACACCCCCTCCACACC
CTCTTTCCCCAACCTCGTGTTGTTCGGAGCGCACACACACACAACCAGATCTCCCCCAAATCCA
CCCGTCGGCACCTCCGCTTCAAGGTACGCCGCTCGTCCTCCCCCCCCCCCCTCTCTACCTTCTC
TAGATCGGCGTTCCGGTCCATGGTTAGGGCCCGGTAGTTCTACTTCTGTTCATGTTTGTGTTAG
ATCCGTGTTTGTGTTAGATCCGTGCTGCTAGCGTTCGTACACGGATGCGACCTGTACGTCAGAC
ACGTTCTGATTGCTAACTTGCCAGTGTTTCTCTTTGGGGAATCCTGGGATGGCTCTAGCCGTTC
CGCAGACGGGATCGATTTCATGATTTTTTTTGTTTCGTTGCATAGGGTTTGGTTTGCCCTTTTC
CTTTATTTCAATATATGCCGTGCACTTGTTTGTCGGGTCATCTTTTCATGCTTTTTTTTGTCTT
GGTTGTGATGATGTGGTCTGGTTGGGCGGTCGTTCTAGATCGGAGTAGAATTCTGTTTCAAACT
ACCTGGTGGATTTATTAATTTTGGATCTGTATGTGTGTGCCATACATATTCATAGTTACGAATT
GAAGATGATGGATGGAAATATCGATCTAGGATAGGTATACATGTTGATGCGGGTTTTACTGATG
CATATACAGAGATGCTTTTTGTTCGCTTGGTTGTGATGATGTGGTGTGGTTGGGCGGTCGTTCA
TTCGTTCTAGATCGGAGTAGAATACTGTTTCAAACTACCTGGTGTATTTATTAATTTTGGAACT
GTATGTGTGTGTCATACATCTTCATAGTTACGAGTTTAAGATGGATGGAAATATCGATCTAGGA
TAGGTATACATGTTGATGTGGGTTTTACTGATGCATATACATGATGGCATATGCAGCATCTATT
CATATGCTCTAACCTTGAGTACCTATCTATTATAATAAACAAGTATGTTTTATAATTATTTTGA
TCTTGATATACTTGGATGATGGCATATGCAGCAGCTATATGTGGATTTTTTTAGCCCTGCCTTC
ATACGCTATTTATTTGCTTGGTACTGTTTCTTTTGTCGATGCTCACCCTGTTGTTTGGTGTTAC
TTCTGCAG
-Exemplary Oryza sativa Actin 1 promoter (OsAc1)
SEQ ID NO: 2
TCGAGGTCATTCATATGCTTGAGAAGAGAGTCGGGATAGTCCAAAATAAAACAAAGGTAAGATT
ACCTGGTCAAAAGTGAAAACATCAGTTAAAAGGTGGTATAAAGTAAAATATCGGTAATAAAAGG
TGGCCCAAAGTGAAATTTACTCTTTTCTACTATTATAAAAATTGAGGATGTTTTTGTCGGTACT
TTGATACGTCATTTTTGTATGAATTGGTTTTTAAGTTTATTCGCTTTTGGAAATGCATATCTGT
ATTTGAGTCGGGTTTTAAGTTCGTTTGCTTTTGTAAATACAGAGGGATTTGTATAAGAAATATC
TTTAAAAAAACCCATATGCTAATTTGACATAATTTTTGAGAAAAATATATATTCAGGCGAATTC
TCACAATGAACAATAATAAGATTAAAATAGCTTTCCCCCGTTGCAGCGCATGGGTATTTTTTCT
AGTAAAAATAAAAGATAAACTTAGACTCAAAACATTTACAAAAACAACCCCTAAAGTTCCTAAA
GCCCAAAGTGCTATCCACGATCCATAGCAAGCCCAGCCCAACCCAACCCAACCCAACCCACCCC
AGTCCAGCCAACTGGACAATAGTCTCCACACCCCCCCACTATCACCGTGAGTTGTCCGCACGCA
CCGCACGTCTCGCAGCCAAAAAAAAAAAAAGAAAGAAAAAAAAGAAAAAGAAAAAACAGCAGGT
GGGTCCGGGTCGTGGGGGCCGGAAACGCGAGGAGGATCGCGAGCCAGCGACGAGGCCGGCCCTC
CCTCCGCTTCCAAAGAAACGCCCCCCATCGCCACTATATACATACCCCCCCCTCTCCTCCCATC
CCCCCAACCCTACCACCACCACCACCACCACCTCCACCTCCTCCCCCCTCGCTGCCGGACGACG
AGCTCCTCCCCCCTCCCCCTCCGCCGCCGCCGCGCCGGTAACCACCCCGCCCCTCTCCTCTTTC
TTTCTCCGTTTTTTTTTTCCGTCTCGCTCTCGATCTTTGGCCTTGGTAGTTTGGGTGGGCGAGA
GGCGGCTTCGTGCGCGCCCAGATCGGTGCGCGGGAGGGGCGGGATCTCGCGGCTGGGGCTCTCG
CCGGCGTGGATCCGGCCCGGATCTCGCGGGGAATGGGGCTCTCGGATGTAGATCTGCGATCCGC
CGTTGTTGGGGGAGATGATGGGGGGTTTAAAATTTCCGCCATGCTAAACAAGATCAGGAAGAGG
GGAAAAGGGCACTATGGTTTATATTTTTATATATTTCTGCTGCTTCGTCAGGCTTAGATGTGCT
AGATCTTTCTTTCTTCTTTTTGTGGGTAGAATTTGAATCCCTCAGCATTGTTCATCGGTAGTTT
TTCTTTTCATGATTTGTGACAAATGCAGCCTCGTGCGGAGCTTTTTTGTAGGTAGA
-Exemplary Panicum virgatum L. Ubiquitin 2 promoter (PvUbi2)
SEQ ID NO: 3
GAAGCCAACTAAACAAGACCATAACCATGGTGACATTTGACATAGTTGTTTACTACTTGCTTGA
GCCCCACCCTTGCTTATCGGTTGAACATTACAAGATACACTGCGGGTGGCCTAAGGCACACCGT
CCGAAACCGGCAAACCAAGCCTGATCGCCGAAATCCAAAATCACTACCGGCAATCTCTAAAGTT
TATTTCATCCTTATATGACGAGGAAAGAAAAGAAGAGAGAAATAATATCTTAACTTCTAAATCA
GTCGCGTCAACTTTCTCGGCTAAGAAAGTGAGCACTATCATTTCGCAGACCATGTCATGAGTGC
CGACTTGCCATATCTTATTATATTCTTATTTATTTAATTATAATCCCATTGCAATACGTCTATT
CTATCATGGCCTGCCACTAACGCTCCGTCTAACGTCGTTAAGCCATTGTCATAAGCGGCTGCTC
AAAACTCTTCCCGGTGGAGGCGAGGCGTTAACGGCGTCTACAAATCTAACGGCCACCAACCATC
CAGCCGCCTCTCGAAAGCTCCGCTCCGATCGCGGAAATTGCGTGGCGGAGACGAGCGGGCTCCT
CTCACACGGCCCGGAACCGTCACGGCACGGGTGGGGGATTCCTTCCCCAACCCTCCCCACCTCT
CCTCCCCCCGTCGCAGCCCATAAATACAGGGCCCTCCGCGCCTCTTCCCACAATCTCACATCGT
CTCATCGTTCGGAGCGCACAACCCCCGGGTTCCAAATCCAAATTGCTCTTCTCGCGACCCTCGG
CGATCCTTCCCCCGCTTCAAGGTACGGCGATCGTCTCCCCCGTCCTCTTGCCCCATCTCCTCGC
TCGGCGTGGTTTGGTGGTTCTGCTTGGTCTGTGGCTAGGAACTAGGCTGAGGCGTTGACGAAAT
CATGCTAGATCCGCGTGTTTCCTGATCGTGGGTGGCTGGGAGGTGGGGTTTTCGTGTAGATCTG
ATCGGTTCCGCTGTTTATCCTGTCATGCTCATGTGATTTGTGGGGATTTTAGGTCGTTTGTCCG
GGAATCGTGGGGTTGCTTCTAGGCTGTTCGTAGATGAGATCGTTCTCACGATCTGCTGGGTCGC
TGCCTAGGTTCAGCTAGGTCTGCCCTGTTTTTGGGTTCGTTTTCGGGATCTGTACGTGCATCTA
TTATCTGGTTCGATGGTGCTAGCTAGGAACAAACAACTGATTCGTCCGATCGATTGTTTTGTTG
CCATGTGCAAGGTTAGGTCGTTATCTGATTGCTGTAGATCAGAGTAGAATAAGATCATCACAAG
CTAGCTCTTGGGCTTATTATGAATCTGCGTTTGTTGCATGATTAAGATGATTATGCTTTTTCTT
ATGCTGCCGTTTGTATATGATGCGGTAGCTTTTAACTGAATAGCACACCTTTCCTGTTTAGTTA
GATTAGATTAGATTGCATGATAGATGAGGATATATGCTGCTACATCAGTTTGATGATTCTCTGG
TACCTCATAATCAACTAGCTCATGTGCTTAAATTGAAACTGCATGTGCCACATGATTAAGATGC
TAAGATTGGTGAAGATATATACGCTGCTGTTCCTATAGGATCCTGTAGCTTTTACCTGGTCAAC
ATGCATCGTCCTGTTATGGATAGATATGCATGATAGATGAAGATATGTACTGCTACAATTTGAT
GATTCTTTTGTGCACCTGATGATCATGCATGCTCTTTGCCCTTACTTTGATATACTTGGATGAT
GGCATGCTTAGTACTAATGATGTGATGAACACACATGACCTGTTGGTATGAATATGATGTTGCT
GTTTGCTTGTGATGAGTTCTGTTTGTTTACTGCTAGGCACTTACCCTGTTGTCTGGTTCTCTTT
TGCAG
-Exemplary Panicum virgatum L. Ubiquitin 1 fusion promoter (PvUbi1 + 3)
SEQ ID NO: 4
CCACTGGAGAGGGGCACACACGTCAGTGTTTGGTTTCCACTAGCACGAGTAGCGCAATCAGAAA
ATTTTCAATGCATGAAGTACTAAACGAAGTTTATTTAGAAATTTTTTTAAGAAATGAGTGTAAT
TTTTTGCGACGAATTTAATGACAATAATTAATCGATGATTGCCTACAGTAATGCTACAGTAACC
AACCTCTAATCATGCGTCGAATGCGTCATTAGATTCGTCTCGCAAAATAGCACAAGAATTATGA
AATTAATTTTACAAACTATTTTTATTTAATACTAATAATTAACTGTCAAAGTTTGTGCTACTCG
CAAGAGTAGCGCGAACCAAACACGGCCTGGAGGAGCACGGTAACGGCGTCGACAAACTAACGGC
CACCACCCGCCAACGCAAAGGAGACGGATGAGAGTTGACTTCTTGACGGTTCTCCACCCCTCTG
TCTCTCTGTCACTGGGCCCTGGGTCCCCCTCTCGAAAGTTCCTCTGGCCGAAATTGCGCGGCGG
AGACGAGGCGGGCGGAACCGTCACGGCAGAGGATTCCTTCCCCACCCTGCCTGGCCCGGCCATA
TATAAACAGCCACCGCCCCTCCCCGTTCCCCATCGCGTCTCGTCTCGTGTTGTTCCCAGAACAC
AACCAAAATCCAAATCCTCCTCCTCCTCCCGAGCCTCGTCGATCCCTCACCCGCTTCAAGGTAC
GGCGATCCTCCTCTCCCTTCTCCCCTCGATCGATTATGCGTGTTCCGTTTCCGTTTCCGATCGA
GCGAATCGATGGTTAGGACCCATGGGGGACCCATGGGGTGTCGTGTGGTGGTCTGGTTTGATCC
GCGATATTTCTCCGTTCGTAGTGTAGATCTGATCGAATCCCTGGTGAAATCGTTGATCGTGCTA
TTCGTGTGAGGGTTCTTAGGTTTGGAGTTGTGGAGGTAGTTCTGATCGGTTTGTAGGTGAGATT
TTCCCCATGATTTTGCTTGGCTCGTTTGTCTTGGTTAGATTAGATCTGCCCGCATTTTGTTCGA
TATTTCTGATGCAGATATGATGAATAATTTCGTCCTTGTATCCCGCGTCCGTATGTGTATTAAG
TTTGCAGGTGCTAGTTAGGTTTTTCCTACTGATTTGTCTTATCCATTCTGTTTAGCTTGCAAGG
TTTGGTAATGGTCCGGCATGTTTGTCTCTATAGATTAGAGTAGAATAAGATTATCTCAACAAGC
TGTTGGCTTATCAATTTTGGATCTGCATGTGTTTCGCATCTATATCTTTGCAATTAAGATGGTA
GATGGACATATGCTCCTGTTGAGTTGATGTTGTACCTTTTACCTGAGGTCTGAGGAACATGCAT
CCTCCTGCTACTTTGTGCTTATACAGATCATCAAGATTATGCAGCTAATATTCGATCAGTTTCT
AGTATCTACATGGTAAACTTGCATGCACTTGCTACTTATTTTTGATATACTTGGATGATAACAT
ATGCTGCTGGTTGATTCCTACCTACATGATGAACATTTTACAGGCCATTAGTGTCTGTCTGTAT
GTGTTGTTCCTGTTTGCTTCAGTCTATTTCTGTTTCATTCCTAGTTTATTGGTTCTCTGCTAGA
TACTTACCCTGCTGGGCTTAGTTATCATCTTATCTCGAATGCATTTTCATGTTTATAGATGAAT
ATACACTCAGATAGGTGTAGATGTATGCTACTGTTTCTCTACGTTGCTGTAGGTTTTACCTGTG
GCAACTGCATACTCCTGTTGCTTCGCTAGATATGTATGTGCTTATATAGATTAAGATATGTGTG
ATGGTTCTTTAGTATATCTGATGATCATGTATGCTCTTTTAACTTCTTGCTACACTTGGTAACA
TGCTGTGATGCTGTTTGTTGATTCTGTAGCACTACCAATGATGACCTTATCTCTCTTTGTATAT
GATGTTTCTGTTTGTTTGAGGCTTGTGTTACTGCTAGTTACTTACCCTGTTGCCTGGCTAATCT
TCTGCAGATGCAGATC
-Exemplary Oryza sativa Cytochrome c gene promoter (OsCc1),
SEQ ID NO: 5
GAATTCGGATCTTCGAAGGTAGGCTGCAGTTCTTGAATTGTTGAATTATTATTATCTTCATCTT
CATTCATCTGTAACTACTGATTCATCTGGTTTGTTATTACCGATCGTAATGCCGTTGTTTTGTC
AAAAAAAAAAAAGGAGATCGGTTTGTTATTACCGATCATAATGCTGTTCTTTTATAAAAAAAAA
ACATGGATCTATTGGCATAATCTTTTTGCGCCAGGTACTCCGACCATTACTCGGTTACCGACGA
AAGCCGGTGAGATTTGGATAAACTTCGCCAAAAATTTAAATTTCCGTTTGATCTCTCAAACGTG
GGCTGGTTTAGGCCTGTTTAATGTTTAGACACATGTATGGAGTACTAAATATTAATAAAAAAAA
TAATTACACAGATCGTGTGTAAATTGCGAGATAAATCTTTTAAGCCTAATTGCTCCATGAACAA
TGTGGTGTTACAGTAAACATTTGCTAATGACAGATTAATTAGGCTTAATAAATTCGTCTCACAG
TTTACAGGTGAAATATGTAATTTATTTATTATTAAGTCTATATATAATACTTTAAATACGTGAC
CGTATATCCCGATGGGAGACACGTAAAACTTTTTAACCAAGTTCTAAACACAACCTTGCTTCAC
AGTTTCTTGATCTCTATGGGTAGGGGTGGGCAGAAAAAGACCGAACCGAAAGACCGAACCGAAA
AGGCCGAGACCGAGACCGAAAAGATCGAGACCGAGAAATTCGGTCCTAGGTAATGAAAGACCGA
ATTTTGTTCGGTCAATTTGGTTAGTTTTCTCGGGTAACCGAATAGACCGAAAAGACCAAATTAT
CAGAAAATATCTAAATACAATCTACAACCCACTATGTTTAATAGGATTAAACTCTAATTTTTTA
CATCCCTACTTCTTTTAGGCATGCAACCTAATAAGAGTCTTTACTCATAAGTGCTTACGAAATT
TTTTTGTGATTTTTGTGTTGAAAATTTCCATTATTTCTTTGCATATATGAAAATGTTGTTGAAT
TTCGGTCAGGACCGAGACCGAGACTGAATTTGTCAGTCCTAACATTTTTTCACCGAAATTCAGT
CTTCACTTTTCAAAGACTGAAAAGACCGAAAGACTGAAGACCGAGACCGAAATTTTCGGTTAGA
CCGAATGCCCACCCCTATCTACGGGCTTGATAAGATCAATAACCGTAATTACCGAAGCGGTTGC
GTGACTTGCTGTTGCATTTGTCAACCCTAACATAGTACTACCTCCGTTTCAAGGTTCCGTTTCA
GAGTTTGTAAAACTTTCCTAGTATTAACCCATGTTTTAACTTGCAACGGGAGGAAGTTAACATC
CTATACGCCTGAAATCCCTTTAAAAAAAAAGAACATTTATACGCTGGAACCGATTCTGAACCGG
TCCGTCCACCCACCGACCCACCAACGGTGCGATTTCCACCGTCCACCAAACGCGAGCCGCCTCC
ACCCTCCACCTATCGAGTCAAAGACGACGACTCTACCAGAGCACGTGGACCCGGTCCACGAACG
GAACGCCCTTACACCGAATGGGCCGTTGGGTGTCCACGCCTCCCACACCCACACCCCCCTTGCC
TTTTTCTGCAAGACACGGAAACCTTCTGGAACCGCGTGGATTCCCCGAAACGCCCCTGCCCCCA
CGCTCCACCCGTTCAATAATTCTAGGGGTATTATCGTAGTTTCGCCACCTGCCCTTCCGCCGCG
CTGGTGTATACTAGGGCACGCGCTCCTCGGAATCGCCACGAGCCCACGAGCCAGAAAAAAAAGG
AAAAAAAGAGAGTCGTAGTTCGCCTCTTCTTCCTCCTCTCGTTCTCGCGGCGGCGGCGGAG
-Exemplary Epipremnum Aureum Ubiquitin promoter (rrEaUbil or P1)
SEQ ID NO: 6
ACAGAGTAATCCTTCAAGACACATAATAACTCACGAATGTAAAGAACTACAAACACACAAAATT
GTTCAAAAAAATTTATGCAAGAAATTTTTTAAGTTACATTATAGCACATTCACATAAGTGAGTG
TCAAATTGATGGATAATCTCCTATATTTTATAAAAAATTACACTCACATGAGTACATGTTATAA
TCTAATAAGAAATCATTATAGTATATAAATTATTTCTCATGTTTATGATAGCACGCACCACTTG
CAACACGTAAAGTATGTACGTGACTACATGTACAAATCTAAATAATGTTGGGGTAAGATAAAAA
TTTAACAAATTTAACATGTAAATACTTTTGGGTCAGACTTAATGCATCGTTTAAGAAAAGCGAT
GCTGGATCGCACACCCATGATCAAATAATTTCTTGTAAATATCTTTTTGAAAAATTTTAAGTTA
ATTAAATATACTCCCGTTAAAATATTTTTTTATAAAAAATCTGCTACATAAATGTCATTTATAT
CCCCATTGCATATGTATATATACATATATATACCATATATGCTGGTTATATATAAAGAGATATA
TTTTTAACAAAGTAATTATTTTTAACTGACAGTTATTGGTCTGGGGCAAATTTAATTTAACAGG
GTATATATGCAATTTACCCAAAACTTTTTAATCTTTTCCCGTGGGGCGAAGGAGCAGACCGGCT
CCGATCCAAACATTCGCCCTCGTATTCCGTCTCCTCAATCTCTCTCTCTCTCTCTCTCTTTCTT
CGCTCCCTCCTGCAAGCAAAAGCCAATATTTTTCTTCCTCCAAATCCCCCTTTCCTCTACAAAC
AACACCCCTCACTGCTTCTCTTGCTTCTCTCCCCGCCTCAGAATCACCAGATCGCAACTCGATC
TAGGGTTTAGAACCGGTACGTCTCC
-Exemplary Epipremnum Aureum Ubiquitin promoter (rrEaUbi3)
SEQ ID NO: 7
GGGGTGCGACAACATTACCTAGTTCATTAGTGGGACCATCTGCAGATTGAGGACTCTTGGATCA
TCCGAAAGTAGTTCCAGTGCCTTGACTCAGACTTATTAGAGTAACACTAGAGCGGCACCGACCA
TTTCTCGACGGGATCGAGTTCTTTCCAGTTAGGAGGAGTTGGTGGAGACACTAAAAATAGGGTT
CGTTTTGACCCTGGGTGGGTCTGCAACAGACGAGAATGTGCGAAAATGACAATGACATCACTTT
AATTTGGAGACGAGTAGTGGGCCCAGTAAGAATTTTGTGGTGCCATCATTATTAAGCATGTTAA
GGTTGGGAGTCTTTTGATACCTTATTGGGCTTATTTGGGCTTAGTTTTATTTTTTTTTTCTTCA
TATTTTTTATATGATTTTCATGCATTTTTTTATGTGTGAGGAATATTTTGGTCATAAAATGTCT
TTTACAGTTAGAGTTATGAGAGAGTTTATAAATATGTTCTATAACTCTCTTTTTTAATTATTGG
AAAATCTTGTTGCGAATTTTGAGTATTTTATTGTACTCTATGAGAGAGGTTGAGAGGACCGCTA
CTTACGGTCATCCGCGAGAGACGGGGACTTACATTCCTCATCGCCCACCCCTTTGCTGCCTTTG
TGACTGTGTTCCTCGTTAAGAAGTCTGATCCCTGAAAAGTTGCTAAAGATACCTCTATCACATC
TGACGTGTTGTGAGGATCGTAATGGTGTAATCACAACTCAAATCAGATGTCGGACGGGCTTGAT
TTCATACTGGTAGATTCTTTTGGAACCCGTGATTGCACAACGTATGGCTGGGGGGGTACGTGTC
GTCGTGGCACTATGTAAGGCAAGCTGAAGTGAGCATAAACAACAAGTAGACCTCGATGGATGAG
TTTGTCATCTTCAGGCATTCATCAATGTGGACGC
-Exemplary Epipremnum Aureum Ubiquitin promoter (rrEaUbi4)
SEQ ID NO: 8
GCAAGTTGCGTAATCGTGCTCCGTTGCTGAGTGGTTTGTTTTGGACTCCTGGTTCTGGCTCGTC
AGACAACTGGTAAACATAGAAATAATCAACTAAGCTGCAAATTTCCCGCAAGGGAAGTTGGCGG
CAGACAATTGAACTGTAACATTTGAATGTAATGGTTTTTCGGTTGTTGACAGGATAATTTTAGT
TAACACCCCGGCTCTCTCACCCGGAGTTCCTGCCTGTGCCTTGCGGGCATTGGGCTTTTGAACT
GTGTTTGGACTCATGGAATTGCATGAAAACTTGGAGCGTGAGGTTGCACGTTAGAAGTGTATAG
AAGTGCCTTAGGAGTTAGCTCCGGGTGTGGGA
-Exemplary Epipremnum Aureum Actin promoter (rrEaAct1)
SEQ ID NO: 9
TCTGTTGTGACATGTGACGTGAATCTAAAGAAACACTCGCTATTTGCATTATTTTTCTTGTATT
TTCAGTGAAGCAAAGTGTCAAAGTTGCCTATCGTTGGTCAAGATCCTGGATCTGTTGGGGATCT
CTCCTTACATTGCAATTTCCTCTTGTCCTTATTGTTTTAATTTCGGAAAGCGCTATTTGTTGCT
TGCTTTGTTGCAGTTTACATCATCCCTTCTTGATGCTCTTTGGGGGGAAATCTCTCTGGGACAT
TCGATAATATTTGGAAAAAAATAGTCTGCGAGCCAGAAGCCCCAGTGCGCTCTCGTTTGTTTTT
CGTCTCATGCTTCTTAATCTTGTATTTGGCATTTGGGAAGAGTGACACAGGATATGCTATCTAA
TTAGTAAATGAATGTGTTTATCGTGCGGACAACTAATTATTCAGATGGATGAAATTCTTGAAGA
TTTATGTTAAGAATAAATCATTATGCAATAATTTCCTAAATGTCAATTGATATTGCATCGGATT
TCACATGCACCAGTAAAACTAGTACTTACCTGTGGTTCATGACAAACACGATTTTTTTTAATTT
TTCTAATGCAATTTACTTTTTCTGCTCATACTTTCTCTTAAAGTAACATCCATCTCCACTTGTT
TTTTTTTCCTTTCTCAAATATATCTTGATCCACACTTACCGACAAGCCTGTACTGGTTTATCTG
ATTGTTAAATTTGATGTTACATTTGAATGGGAAGAGATATCATGTTAGTTCGGTTCTAGCATTA
AAATGCCTAGTACATCTTACTCCTTTTGCAGAATGACTTTCTTTATACATATGGTACGTTATTT
TTCTTGAAATGGAGCTTGCCCAAGCAGAATTTCTTTTTTCATGGATGATGGTTGTCGTTGGTAG
TTTAATTTTATCATTAACCTTTCACGTCTTACATATTTCTCAGATATTGGTGAATATTTTAATC
TGAAACGTAAAGTGAGCAGGTGTAGA
-Exemplary Epipremnum Aureum Actin promoter (rrEaAct2)
SEQ ID NO: 10
ACACCATCACCCTCATTGGTTTCTGTAGCATGACTCTGAGCTACGATGGAAGATCCAAGTTCCA
AAATAAAAATAGTCCCTGGTGTCACTATTGGGTCGCTCAAGCAAGGCATATATTGTCTAAGTTG
ACCTGAAAATTGCATGACCAAATCTGATTCCCGCTCACGGCCCTGTCCGCGACGTCACTCGTGA
AACTCCCTATTAGAGGGAGAGTGGAGCATCATGCTTGGAAGCTAAAAAAAAATGGATGATGTCA
AAATTCCAAACTAACAATAAGTAATGAGCTGTATTGGGCAAATAATACTAATATAGAAGTAGTA
AGTAAAAGAGAGAGAAAAAAGAGTCAATAAAAAAAATGCAACAAAAGGTTTTGTGCTTACCGAC
CGCTGTCCGTGGCACTTCCCGGTTCGTGGGGGACATTTGTTGGCAAATATCTTTTTTATTATTA
TTCAAAAAAAATGAAAAGGAAGGGAGATAAGAAAAGACAAGAGACTGCTCTCCCACACCTTAAT
GCAACTCAGGTTGGTTCACTTATGGTGCAACACAAGGTAACCTGCAATCAAAAGGTCTGGGCAG
CTGGATTTTGTGCTGTCTTACTTTAGAAGCACAACTCTTTGACATATGCTTTGGTGGAATTTAT
CAAAGGAAAAGCTCCTGATGTTGTAAACAGTGGGTCAATAACACAACAGGCTAAAACAGATTTC
ATGAAAAATTCATTCTCTGGTCTGCTATAGAAAAGTTCTTCACAGTGATTTTGGGGCTACCAGA
TGTTCAGAGGTGGTATTCAGCTAGCGGCAATTTCAAGCTGGGTTGCAGTTTGAAGGCAGAAAAG
AGACAGGCTGTTCTTTGCCTGATCAGGGATTGTCCCCCATCTCTCTCCCTCTGTCTTTTCTCTC
CCTCCTGCACTCCCATCAGAAAATAGCAGGGAGAGAGAGACTGATGGGTCTTTCCCTCTCTCAC
TGATTTTTCCCTTTCTCCTGGTTTTCTCT
-Exemplary Epipremnum Aureum Histone H3 promoter (rrEaH32 or P7)
SEQ ID NO: 11
ATGGCTGCATTACCTGACGTACAATATTATTGGTAGGTAATTCGAGATTAACTATGAAATATGT
ATATGTGTCTCACAACTAAGTAATGGCCAACTTAGTTAACCAGGTTATGAACAAGTTAAAGTTG
GTGTCAAACTCTGGATTAACTTCAGAGTAACCACTCTCTACTTAGAACCCAAAACTTATGTAAG
TTAATACTAATGAGTAATCTCTGGACTAACCCACCACACCAATTCATGACTTTTGGAAGAAAGA
TTACTTATTAATCCGAATAATTTGGACCCCCTTTTTGAAAATAATTATTGAGTTAATTCTGAAC
TATTAAATATTTCATATTATTAATAATCATTTTAAATAAAAGCTGCTGATCTTAGTTGTAATTT
TTTTTACTATTAACAAAGAGAGAGATAAACGCATTTTTTTCTATTTTTATACCAAAATTAACCC
ATATTCAAATTTTGGGGATGACACATGAATTAAGCTAGTTTCTCATTAGAAAAAGATCTTAGCC
TTACTTATTAGGGGTACATAGATAATTTAATTTTTTTAAATGTTTTCACGTAATTTCAAACCAT
TTAGGCCAAAGCGGGCCGAATTCAAATTCGTGGGCTCGGTGTCACGTTGGTCCAGCCAGAGCAG
TGTTATCAGCTTCCTACCTGGTGAAGGTACGCCATTGGCTGTTGTCCGACGACGCGGATCAAGT
TGCATAAACAAATTCGCACCGTCCGATGAAAGCGAATGATCCCGATTCACTCAAGGGGCCCCCG
CTGCGGCAGCGGCGGAGAAAATTTCGAACTCTCCGCCAAAAGGGCTCCTCTCTCTCTCTCTCTC
TACAAATACTCGCCAAAGGCTCCCCCTTTGTTCTACCCAAGCAGTCCTCGCTGCTCCAGATCGA
GAGGCATCCAGAGAGCGTCCGAAAGAA
-Exemplary Epipremnum Aureum Histone H3 promoter (rrEaH31)
SEQ ID NO: 12
TGTTACAAAACAGAAGAAATTTGACATATGTGTTGAACATAATCTTGTCCTAATATTTTTTTAT
TTTTTTTAAAATTTTAAAGTACTTAAAAATATTATCTCTTAAAATCAACGTCCATCACACAATT
TGTAAATTTGGACCAAGTCAACCTGAGTTGATTGACTTAGTTCATATTCAATTATTTAGTATAT
ACGATTCAATACAAATTATTTAAATAATAATATAATATTTAAAATATAATTTACATATTTTATA
AAAATTAAAAATAATAAAAATTTAAATATGTGACTTAATAAGTCACAAGAGTTTTGATATGTGG
ATAAAAGTTTCTATAGACAAACAAGATTTTTTTGAATAAAAATTATCTACTAAATTGTAAAAGT
TTTATGAGATTTTAAGATTTGTTATTTATAAACATAAAATTTTTAATGTTAAATAAAATAAAAT
AATTGATGAAAATTTAAATTATCCTATTATATTGTCAAAAAATTCACAAGAGAAGAGTGGCAGT
CAAAAGTTATCCTCGAATTATTTTCTTAATATAGATAAAAAAAAGATCTCGAGAGAATTTAAAA
TTTAGAAACCCCTGGCCCACCCTAGCCCAGAAAGCTCGCCAGCCGCGCTGGCCGGGCCCGCACT
TACGCTCCCAAGAGGGAGCTTGGCCAAGGTCGAAAGTGACGGCGATCGCGATCCGCGTGCTATT
CCTCAGGATCATCTCAACCGTTCTTTGAGACAAATCGACGATCTCGACTAACCACCGAGAAATT
CAAAAGTTCCAAAACCGGCTCCCGCCTTTCGTGCGCCTACAAGTATCCATCCCTTCCCTCAGGG
CTTGAATCGTCTCCACCCCTCCGAACACAAAGCATTTCCTCCTGCTGCACCGAAACCCTAGGCC
CTCGTTC
-Exemplary Cauliflower Mosaic virus promoter (2x CaMV35S)
SEQ ID NO: 13
GTCAACATGGTGGAGCACGACACTCTGGTCTACTCCAAAAATGTCAAAGATACAGTCTCAGAAG
ATCAAAGGGCTATTGAGACTTTTCAACAAAGGATAATTTCGGGAAACCTCCTCGGATTCCATTG
CCCAGCTATCTGTCACTTCATCGAAAGGACAGTAGAAAAGGAAGGTGGCTCCTACAAATGCCAT
CATTGCGATAAAGGAAAGGCTATCATTCAAGATCTCTCTGCCGACAGTGGTCCCAAAGATGGAC
CCCCACCCACGAGGAGCATCGTGGAAAAAGAAGAGGTTCCAACCACGTCTACAAAGCAAGTGGA
TTGATGTGATAACATGGTGGAGCACGACACTCTGGTCTACTCCAAAAATGTCAAAGATACAGTC
TCAGAAGATCAAAGGGCTATTGAGACTTTTCAACAAAGGATAATTTCGGGAAACCTCCTCGGAT
TCCATTGCCCAGCTATCTGTCACTTCATCGAAAGGACAGTAGAAAAGGAAGGTGGCTCCTACAA
ATGCCATCATTGCGATAAAGGAAAGGCTATCATTCAAGATCTCTCTGCCGACAGTGGTCCCAAA
GATGGACCCCCACCCACGAGGAGCATCGTGGAAAAAGAAGAGGTTCCAACCACGTCTACAAAGC
AAGTGGATTGATGTGACATCTCCACTGACGTAAGGGATGACGCACAATCCCACTATCCTTCGCA
AGACCCTTCCTCTATATAAGGAAGTTCATTTCATTTGGAGAGGACA
-Exemplary Agrobacterium tumefaciens Nopaline synthase gene promoter
(NOS)
SEQ ID NO: 14
GAACCGCAACGTTGAAGGAGCCACTCAGCCGCGGGTTTCTGGAGTTTAATGAGCTAAGCACATA
CGTCAGAAACCATTATTGCGCGTTCAAAAGTCGCCTAAGGTCACTATCAGCTAGCAAATATTTC
TTGTCAAAAATGCTCCACTGACGTTCCATAAATTCCCCTCGGTATCCAATTA
-Exemplary Agrobacterium tumefaciens Octopine synthase gene promoter
(Ocs)
SEQ ID NO: 15
CTGAAAGCGACGTTGGATGTTAACATCTACAAATTGCCTTTTCTTATCGACCATGTACGTAAGC
GCTTACGTTTTTGGTGGACCCTTGAGGAAACTGGTAGCTGTTGTGGGCCTGTGCTCTCAAGATG
GATCATTAATTTCCACCTTCACCTACGATGGGGGGCATCGCACCGGTGAGTAATATTGTACGGC
TAAGAGCGAATTTGGCCTGTAAGATCCTTTTTACCGACAACTCATCCACATTGATGGTAGGCAG
AAAGTTAAAGGATTATCGCAAGTCAATACTTGCCCATTCATTGATCTATTTAAAGGTGTGGCCT
CAAGGATAATCGCCAAACCATTATATTTGCAATCTACCA
-Exemplary Agrobacterium tumefaciens Mannopine synthase gene
promoter (Mas)
SEQ ID NO: 16
ATTTTTCAAATCAGTGCGCAAGACGTGACGTAAGTATCCGAGTCAGTTTTTATTTTTCTACTAA
TTTGGTCGTTTATTTCGGCGTGTAGGACATGGCAACCGGGCCTGAATTTCGCGGGTATTCTGTT
TCTATTCCAACTTTTTCTTGATCCGCAGCCATTAACGACTTTTGAATAGATACGCTGACACGCC
AAGCCTCGCTAGTCAAAAGTGTACCAAACAACGCTTTACAGCAAGAACGGAATGCGCGTGACGC
TCGCGGTGACGCCATTTCGCCTTTTCAGAAATGGATAAATAGCCTTGCTTCCTATTATATCTTC
CCAAATTACCAATACATTACACTAGCATCTGAATTTCATAACCAATCTCGATACACCAAATCG
-Exemplary Cassava Vein Mosaic Virus promoter (CsCMV)
SEQ ID NO: 17
CCAGAAGGTAATTATCCAAGATGTAGCATCAAGAATCCAATGTTTACGGGAAAAACTATGGAAG
TATTATGTAAGCTCAGCAAGAAGCAGATCAATATGCGGCACATATGCAACCTATGTTCAAAAAT
GAAGAATGTACAGATACAAGATCCTATACTGCCAGAATACGAAGAAGAATACGTAGAAATTGAA
AAAGAAGAACCAGGCGAAGAAAAGAATCTTGATGACGTAAGCACTGACGACAACAATGAAAAGA
AGAAGATAAGGTCGGTGATTGTGAAAGAGACATAGAGGACACATGTAAGGTGGAAAATGTAAGG
GCGGAAAGTAACCTTATCACAAAGGAATCTTATCCCCCACTACTTATCCTTTTATATTTTTCCG
TGTCATTTTTGCCCTTGAGTTTTCCTATATAAGGAACCAAGTTCGGCATTTGTGAAAACAAGAA
AAAATTTGGTGTAAGCTATTTTCTTTGAAGTACTGAGGATACAACTTCAGAGAAATTTGTAAGT
TTGT
-Exemplary Arabidopsis thaliana Actin 2 promoter (AthAct2)
SEQ ID NO: 18
AGGAGTCGACAAAATTTAGAACGAACTTAATTATGATCTCAAATACATTGATACATATCTCATC
TAGATCTAGGTTATCATTATGTAAGAAAGTTTTGACGAATATGGCACGACAAAATGGCTAGACT
CGATGTAATTGGTATCTCAACTCAACATTATACTTATACCAAACATTAGTTAGACAAAATTTAA
ACAACTATTTTTTATGTATGCAAGAGTCAGCATATGTATAATTGATTCAGAATCGTTTTGACGA
GTTCGGATGTAGTAGTAGCCATTATTTAATGTACATACTAATCGTGAATAGTGAATATGATGAA
ACATTGTATCTTATTGTATAAATATCCATAAACACATCATGAAAGACACTTTCTTTCACGGTCT
GAATTAATTATGATACAATTCTAATAGAAAACGAATTAAATTACGTTGAATTGTATGAAATCTA
ATTGAACAAGCCAACCACGACGACGACTAACGTTGCCTGGATTGACTCGGTTTAAGTTAACCAC
TAAAAAAACGGAGCTGTCATGTAACACGCGGATCGAGCAGGTCACAGTCATGAAGCCATCAAAG
CAAAAGAACTAATCCAAGGGCTGAGATGATTAATTAGTTTAAAAATTAGTTAACACGAGGGAAA
AGGCTGTCTGACAGCCAGGTCACGTTATCTTTACCTGTGGTCGAAATGATTCGTGTCTGTCGAT
TTTAATTATTTTTTTGAAAGGCCGAAAATAAAGTTGTAAGAGATAAACCCGCCTATATAAATTC
ATATATTTTCCTCTCCGCTTTGAATACTGTATTTTTACAACAATTACCAACAACAACAAACAAC
AAACAACATTACAATTACTATTTACAATTAC
-Exemplary Solanum lycopersicum Histone H4 promoter (SlHis4)
SEQ ID NO: 19
AGGAGAATATCATTTTTAAGTAAAATTTTGAATTCAAATGTTACGTGTATTATTTAATTCATCA
ATTTGCCTTGTCATAGCGAGTACATTACAAACATCACATATATTTGATTGATTGTCAAAAAATA
TCAAAATATATATCAATTTTAAGAGGTATAGGTGTCTAATATGTACTAGCCCTAATTTAAATAT
CTAAATTAATTATTCGGATGAATCTATATACCATCTTTTTAATGGACACCCAAAATCACACATC
AAACATCATATACATGTTGAAAACATATTATTGATATAGCTACATATATGTTTTAATATAAATA
AAAGACGAGTCATATATTCAAAAATTAAGAATCAAATAATTTTAATTTATTTAATATTCAAAAC
TTAATACTATTTAAATTTAGATATTCTAATTTTAATACACGTCTGATAAAATAGATGAGGACTA
AATAAATAATTTGAGACTATCTTTTCTTTATTTGGCGGCCCACAAATAATTTAGATTCTCGTAA
CCCCCTCTTTTTCTCTCACTGAAAAAGCACAATCCGTGTCCAAACACAAAGAAGCACTCGACAC
CGTAGATCTCCATTCAGATCAACGGCTTATATTCAGTTTTCTCCATTCACGTGGATCGACATTC
TTATCCGTCCGATTATCAATAAATTTCCCAAAATTTAGCGGCCATGATTTTAACCCCGCCTCAT
TTCAAACCGCCCACGAAATCCTCGACGCCCAAATTCACCAACTATAAATAGCCACCACCATCCC
CTTCATCAATCATCAAATTTCATAACCCTAGAATCATCACCTTTTTCAAATTTC
-Exemplary Arabidopsis thaliana Light-harvesting chlorophyll-protein
complex II subunit B1 Promoter (AthLHB1B1)
SEQ ID NO: 20
AGGAGATATGACTGGTAAGTTTTTCTTGCCAATACGAATTAGAAAACATGTCTTTGAAGATGAA
CTGTATTTTTTTTTTTTACTTTGTTGTCATTTTAATGTACTTTCTTATCAGGATTAAATCTTCT
GTAATTTAGAGTAGTTTTTTTAACAAGATAATTAACAAACTTAGAGTAATGAAAATTGAGATGT
TCAGTTTTCACTCATATTTCACATTTTGGTGAAAGAGTGGGTAGTATGCAACGTTCTAAGTATG
TTTGGACTTTGTATCATGTTGTTTTGATTCTTTGACGACATGTCTATTTGGGAAACACCAATGA
CGTGTACCTTGAGACTGATACGATTCAAAGGGATAGAAACACGTCAGATTTACAAGTGGCACCT
CTTCAATGGACAATGGGTATTCCAATATGCTAAGATGCTACGAGATATCTAATTTATCTAACAC
AACTCAATTCCAAACCAAAAATCTGATGCCAGCTCGACAAGACAAAAAATCTAAGCTCAAAAAT
GTCAACAACCAATAGAAATCAAGGCATTGACGATATCACGAGATAAGCAAATTAAATCTTCAAG
TTTTGCAATTCATATGTACGTTATAAATACCCAAAAACCTCACCGTAACCTAGCTATCCAATTT
CATCACATCTTATTAACTAAAGAGCCTTTTACTTGCGCCACACTCTCACCGC
-Exemplary Epipremnum aureum ribulose bisphosphate
carboxylase/oxygenase activase 2 promoter (rrEaCons1)
SEQ ID NO: 21
ACCTCAACCTTCGCTCACAGTGAAGGCTTGAAACTCGCTTTTTAACATTGTAAGTGGGCTGATT
TTGAACTCATCTCATCGTAAATCTTTAAGCTTTGACTTCCCACGATGTTGTCCAGTCTATTAGA
TTTTTTATGGTTTTTTTTTCTTTTTTCGCTGAAAGTTCCTACTTAAAATAGTCACCCACTAGGT
ACAGAAGAGTCAGCTACATGAAAAATACCTTAATATAGAAAAACGTATTTATTGTATTAAAATT
TGAACCCTCCCCACTTAAAATGATGCGTACCACTTAGACCTAGTTGAGATTTATTGTTGCACCT
GGGAGAGAGTTGAATAGGGTCCGGATTCCCACTTAGTTTCTCTGGAATCTAGATAGGGCGGTCA
GCTTTATCTTAATTAGTGACAAGGCACTAGTTGGAGTTAGTTTTTATATTGAACATACTCTTAA
ACTTTTAGTTCCCTATTTTGAGAGAAAGTATTTGAAGTAATTTTAAACTTTTGGTTAAATCTTC
CACTTTTGACCAAAAGTTCAAAATTAAAGTTTCCCAAGTTCAAGAAAGAATGGTATCATTAGCC
CATATAAGAACTAAATTAAAATCAGTTTGATTCATTCTTATTAAGCTCCAACATACTCAACAGC
ACAACCAACAGCATGACTTGTGTAAACTGAAAAACTCAGAGAGAGAGAGATAGAGACTCTGAAC
GAGTGGTGCTGAGCAGCAGTGGCTGCTTCATGAAGAGTTTGGCGTGACGACAAAACCATCAAAA
ACACAGAAGAGGAATTTCATTGCCGACAATCACCATGTCTCTGTAATACTGCTGGTCCTGATGA
AATGCTTGAAGGAAAAAAAACTGGCATTAAAGAGGAGGGGAAAAAACCGAAAATTTTAGTGGAG
TCGGGAAGCCCGGGAACCCGAACCATTCCTGGCGTCTGACGTCCTCCGCTGCCGAGAGGATGCT
GTAGCTGATGGGCCCCACTTCCCCACACTCCCCAACTTCCAACGTCAGGACACGACTCTATCTG
CGCAGAAGCAACCAACCCTGATGCGCCACGTGTCGCCCCACCCCAATCCGCAGTGTGTGGCCGT
TGTGGCCCTCGCGATCCAATCCACAGGATGCTTCACTCTCCTCCTCTCCTCCGCAAGCCAAACG
GGAAAATAACGGAGCAGGGCAGACTCCAGAGCCTCCGCAGGCCGCTTTATATATAACTCGCCCT
CCCACGCCTCCTACGGTCATCACTGCCGCGAGGAGCTTTGCTTTTGGTGGACGCGGCGATCTCC
CCCCATCTCCTTCTCGGTCTTCC
-Exemplary Epipremnum aureum Metallothionein-like protein type 3
promoter (rrEaCons2)
SEQ ID NO: 22
AGGAACAAGTGCCACCTGAGCCAAGGCGCTCATTGGCGTCTTGATAGTTTCTTTTATGGTATAC
ATGCTGTTGTAAGAATCTTAATGTTTTAAATTTGCATCTGCATGTATATATCCACGTTTTGGTG
TAATATCCACGTCTATACCCTTGTGAAAGGTATCTGTATGCATCCAAGTATAGTTAAATCACTT
TTTAAAATTTACAGCTATGTCCCTTGTAAAGCTATAATGACATTTTTGTGCATCTAGAAAGAGT
ACTCACTCGGGGACTCTTCTAACAGACAAGCACATGATGAGAAATTTGCACCCGCACAATTCAA
ATTTGATTCTGAAAGACTTGCAACTTACAAACTATCTTAAGTACGTACGACCACAAATTATCTC
AAGTGTACTCTTTGTTCCACAAATAACTTTTACATTGACACTATTTAAGGACGACACTGATCAG
AGATAAAATGACAAAATGAAAGGGGACTCATCTAAGTTAGACAAATCCCGAAACTTATTTCATA
TACCCTAAGAACACTTGCCCCCCTAATTAACGACGGTACATGAGTAACATGTTTGCTTTTCACA
TGAATACAAATGGCAGTACATATATGTAAGCTAGCAAGAAGGATATGTGGGTGATAATTATCTG
TATATGGTCCGTATCCACCTCCCTCTCTAGTATCTCCATCACGTAGCCAGAGGTCATCGGATTT
GTACACCAGTTGCATGTGCCTGTGCATCTGTTGCCAGTTGCGTGTGACAGTGCAGCTGTGTATT
GCCACAAAAAAAAAAGGAATAAAAAGGTAGTGCAACTGGGTAACGGTGCAAGGATAGCCGTGTC
TGCCCATCTGAACCCAAAAGGGCGACGACGACGACTCGGGGAGGTGAAAGAAGAGGAACTGGCG
TGAGAGCTGGTGGGGCAGCCCCCCTCCTCTCCACCATAATTGAGATTCCTTTGGAAGCTTCCCC
CATGGAGGCGTGTGCCCGTCACACACAGGAGGCAGAAGCCCTTCCCCTCCATCTCTCCTTGTGC
CGTGTGCGGCTGCCCATCCAACCCCTGGGGCCTATAAATATCGTCGCAGGGGCAGAAGCCCCTC
CAGCATAGCTGAAGCTTGAGTAGTTCAGAGATATAGCTCTCTTTGATCTCCAGAGAGGCTCCCT
CCTGACATCACCACC
-Exemplary Epipremnum aureum abscisic stress-ripening protein 2-like
promoter (rrEaCons3 or P16)
SEQ ID NO: 23
GTTCCACTCGAGGCAGGAAAAATCTCTGGATTTGGACACTTAACCGACCCCCATTAACACCCCA
CCTCACATCAGAGCACGGTTTGCCCACTCAACTTGTCAGGCAAACCACATCTTATCTCAAAAGC
TATGAGTTACAACGTCAGATAACTAATTTAAATAATAATATAAATTTAAAATATAAATTATATT
TTTTATTAAATTAAAAGAATAATATTTTTTAAATATCTAATTTTATCCAATCAAATTCAAGTTC
AACTGATCTATATTAAATAAAAAAATTAATACGAATCCAAATTTTAAGTTGACAAATAAATGAA
TTTTGAATAAAAGAATCACAAATAAAAAATTACGTTTTCTTGGCGTATATCACCATGCTTGTCT
TCGTTTAAGAGATTTAAGCAATCATGGACGTCTGCTTATCCACGGATGTGAAATATTAAATGAT
AAAATACTATATTATCTTATATTATAGAAAAATAAATTTTAAATGAGAAGTGGGTATTTATTAT
GTTTTCATTCAACATACGTGCGAAAGTTTTATCTAGATAGATTAGCGTTAGCATCACTCAAGAA
TTTTTTTTATTTTCTTAACTGCTTCAAAAAAAGAAATATAAAGGGATTGGCCCACGTTAATTAG
CTAGAAAAAGTGGGATTGAAACGGGTGTTATCCACTTCACATTCTGTGAGCGAATCCGATGCGT
GAAGCCCCGCCATCCTGACCCGACCGCTGTTCCCCCCTACCCACGAAGAAGCCGTCTGTCCGTC
TCTTCAATCTCTATACTTCCCCTTCGCCTGCTGCGTACACTCCCGTGGCTATAAATAACCACCA
CAGCCTCTCTGATTTCTTCGTACCCATTACTGCAACACCTCTACAGCTACTAGCCGTGTCGCCC
GCCCCCCCTTAAGGTCATTCTACCACTGCCAGT
-Exemplary Epipremnum aureum RNA-binding protein cabeza-like
promoter (rrEaCons4)
SEQ ID NO: 24
GCAACAATGACGCGGATTCAGCCCGCCAAACAGATACCATTAACTCGGTTCACTTGTTTAAGAA
AGCGTTGTAGATTTTTTTTTAAAATTTATTAATAAAATTTTACCGCCCCCAAAGCCCAAACTAA
TGTTATCAAGTTGGAATCTGAAAAAAAAATAGATTCGAGAGAAAGATATTAATTCAATCAAAAT
ACAAATAATTCATGAAAGGTTCTGAATGTATCGTCGATCTTTAATATAATTAAATATTAATTGT
AAATCATATAAAAACTATTAATTGACTAGITCCAATAGCCAGTCCTTGTCACTCTTGGCTGCAT
TGCCGGGTATCGGATATTGGCACCGCGGAGAACGCGAGAGGTGCCTCACCGCCAACATGGAAGG
CGCTTGCGCCTTTCGGTTGACTCCCGAGGTAAACAAGGGGCCAGGGGCATCCACGTAAACACGC
CCTCCCCCGGGCCCAGGGGTATCCACGTAAACACGCCCTTCAGATATGTCTGTGTCGCTTGCGC
GGTCCCCGCCCCGCTCGTTCCCTTCCCTGTGATAAGCACAAAGCCACGAACCCTGTTCTGGGCC
TAAACGGGCCACCAAACGATCGGGGGATCCAATCCAGCACGAGTTCCACTGTTCCCTCACCCCA
TCTAAATCTTAATTTGCTCCAGCTCCACGAGGGTACCATTACACAGCTCCCGAAAACGTCCACC
AGTTCGCACAGGCTCGTCGAGGGGAACACGATAGTGTCTAGTGCGGGGTCCATGGGCCCATCCA
GTACTGCCGGCCAGTCCACGAAGCCCAACGGGGACCCTGGTTGAACCCAAGCGTGGGGTTACAA
ACGCTCGAG

In certain embodiments, compositions and methods described herein utilize an inducible promoter. Inducible promoters allow regulation of gene expression and can be regulated by exogenously supplied compounds, environmental factors such as temperature, or the presence of a specific physiological state, e.g., acute phase, a particular differentiation state of the cell, a particular growth stage of a cell, and/or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionein (MT) promoter, the dexamethasone (Dex)-inducible mouse mammary tumor virus (MMTV) promoter, the T7 polymerase promoter system (WO 98/10088, which is incorporated in its entirety herein by reference); the ecdysone insect promoter (No et al, Proc. Natl. Acad Sci. US.A. 93:3346-3351, 1996, which is incorporated in its entirety herein by reference), the tetracycline-repressible system (Gossen et al, Proc. Natl. Acad Sci. US.A. 89:5547-5551, 1992, which is incorporated in its entirety herein by reference), the tetracycline-inducible system (Gossen et al, Science 268:1766-1769, 1995, see also Harvey et al, Curr. Opin. Chem. Biol. 2:512-518, 1998, each of which is incorporated in their entirety herein by reference), the RU486-inducible system (Wang et al, Nat. Biotech. 15:239-243, 1997, and Wang et al, Gene Ther. 4:432-441, 1997, each of which is incorporated in their entirety herein by reference), and the rapamycin-inducible system (Magari et al. J Clin. Invest. 100:2865-2872, 1997, which is incorporated in its entirety herein by reference).

In certain embodiments, a suitable plant specific inducible promoter may comprise but is not limited to: an Epipremnum aureum leaf patterning promoter, an Epipremnum aureum leaf age dependent promoter, an Epipremnum aureum salicyclic acid stress responsive promoter, an Arabidopsis thaliana stress response promoter, an Epipremnum aureum auxin signaling responsive promoter, or a combination of any characteristic portion of these promoters.

-Exemplary Epipremnum aureum leaf patterning promoter (rrEaAs21)
SEQ ID NO: 25
GCTCCGTCCCTTTTCCCTTTTCTTTCCATTTCTACCATGCGTGTCAGCGTGTGCGTCCATTGCT
CGAACTGTGTCTGCACGTGTTCATGTGATCATCAGAAGTCTTGTTCGCAGGCCCACCGTTTTCG
ATTTGGAGATCCCCGGACATAATCCGGAAGAGATCTTCTTTTTTAGCACATGAACATACAGTAA
TGCGAGAATGGAAGGAGTGAGAAAATATCCTTTGAATCCCGGTTGCATCCCGAATCCTACCGAG
AAAGAGAGGATCTCTATCTCAAGCAGTGTAAGAAGAGCTCACGGTGGTCTTTCCCGATCATGTC
CGGAGGCATGTGATCTCAAGTGCTGTGGTGCAAGTAATCCCCTTAGAAGGTTATGATCTCCGTT
CCGTATCCATCACCGTCTTTCGTACTTCATGGGTTTCTCTTCCCTTCTCTCTCCTATCCGTGTA
TCTTCTCAGATTTGTATGGGAGATACTGTATGGGGAGGAGTAGAGTCTGGGTTGTATTCAGTTC
CCTCCATTGCCCTTTTAGACAAGAGAAAGGAAAAACAGTGAATTCCATGTGTTCTTCTGTCCAA
CCGTGTCGCCTTGCTGCGAATAGTCCTAGCAATTGCACTGTTGCCATGCCTTCCTGTCACTGTA
AGATGACACTCTACTCTGTGTGTCTTTTTTGGTATTATCTCTAAGGGCAATCCGCACACGTTCC
CGTTCATTTACTTCATGTGGAAAAGAAAAAAGTTTGTTTCTTTCTGAAAAAAATCATGGAAGAT
AATTGTTTTGCCCACTCATTTGCTACTATATATTCTACCTTAATTTGTTTGCAACGGGTCAGGT
TGTTTAAATCTGACTGTTTAAAGGCTCTATCTTTTGGACAGGAATTGATCATATATAAGCAGCC
GTGTGTGGTT
-Exemplary Epipremnum aureum leaf age dependent promoter
(rrEaKan22)
SEQ ID NO: 26
CCATCGCTATTCTTGTATTGTCACGAATGCCACCCCTAGATAATTTATTTGTGAAAATATCTTT
GAAATACAATTTTTGTGCATAAATTCTCAAAAGATGGCATTCATATGAGAATAAGGGTGACAAA
TGCGTAATGTAACAATGACATATTTGTAAAAAAAATTCATATCTAATTTTCCAACATTAATCTA
TCTAAAATATTATAATATCATATCTAATAGATGTTGACCATACGTGAGGCATTTGGCACTAGGC
CTACCCAAGGAGGATGCAAATGTGTTTTTAATGGAGTTACTTTGCACATCTTTTATACAAGGGG
GGCATCGTTACAAAAACTCAAAATTAACTTGTGAGAGGCCGGCTTTATCTTTTTATGGCCCGTA
AAGCGGAAATATGAGAAGTGGAGAAATGGAATAGGAGACAGGAAGGAAGGGATGCACACAAAGC
TAAAATGTTAGATCAGAACTTCACTTTTTATCAAAAAGAAAATCAGTGGGAAAAAGAATAAAAA
AAAAGAATCGAAGCCTTCTCTCTCTCTCTCTCTCTCTCTCTCTCTCTCTCTCTCTCTCTCTCCT
TCTATGTGTGTTTGTCCACACCCCACGTCCACAAAAGAAACATACTCCTACTTTCTCCTCTATT
TCTCTCTCCTGGCAGCCAAGACCATTCATACCGAGTGTCATTTTCCTGCACATACTTCCCCTTC
ATACAAGAAGTAACCACTTCCACTCTCCCCGTTTCAAGACATTTACCTCCCCTCCAATCCCTCG
TTCCCCAACTCCCCTCCCAAAACCTTCCTGTTCATCTAGAACACCCATCTGCTCCACACCTCCT
ACCCTTCCCACACTCCCAACGGGAAGAAGAACTCAGTGTACGAGAAGAAACCCAGAGTCCCGTC
TGCGGCGGCGCAGGCGGAGGGTAGGGAGGGAGGGAGAGAGAGAGTGAGTGTGTGTGTGTGTGTG
TGAGAGAGAGAGAGAGAGGT
-Exemplary Epipremnum aureum leaf age dependent promoter
(rrEaDPA41)
SEQ ID NO: 27
TGCTCCAATTACATTTGCCATCTGAAAATATATGCCACAGTCTGGTTAATTTTTAAAGAAAAAA
AATAATATTCCAGCAGAAGAATGGATCGCTGGATCAAGTTTTTTTTCTGCCCAATTAAAAGTTG
AAATGGTGGTCCAAAATGATTTCTTATTCGGAAAATTGAATATTTTAAAATAATATATATCGTA
CTGACACGTGAGAATAGCGAAAAGGACGAGCTCACATGAGCCTAACCAGATGGTGCATGGTCCC
GGTCCAGCTCTCCCTTCCCGTCTTTGCACGGCTCCAATTCCTCTCCCAGCTTTATCTCTTCCAT
CTCGGTTCCCTTTCATCTCTTCTCCCCAGCTGTAATACGAGAGGAATACCAGTGCAGGTACTCG
CGCTTCGGCGTCTCTGTCCGCCGCTCCTCCTCCTCACTCCTTCACCAGATCTGTTATAAGCTGA
AGCCTCTCAAACCCTAATCTCGAATGTCCCCAGGGGTATGAGCCCATCTGCAGCCTTTCCATCC
CAGAGATCGATGGGAAGCCATCTAATCCTGTAGTTCTGCCTGCTATAGCACTGAGCAGCGGGAG
AGCAGGCCATGCACCGATCCACCCCTTCGGCTGTATCCTCCTCCTCTTCTGATCTCCTCTTCTC
CCCCCTCCCTCTCGTTGTGCAAGCAGTTCAGTGGGATGCCCGCATCTCTCTCTCTTTCCCCCAT
ATTCTCCCCTCCGCCCCCGCTTTCCGTTTCTTTCTCATCTTACAGGTGTAGAGAGAGAGAGAGA
GAGAGAGAGAGAGAGAGAGCTGTGAGTTAACACAGTAAAAGAAGGCGTAGGATTTGCACAGTCG
TCGTCTGTCGTCTGAGA
-Exemplary Epipremnum aureum salicyclic acid stress responsive
promoter (rrEaPR11)
SEQ ID NO: 28
GGAATTCCCACAGAATCAGATTCGGGTACAAATGCGCCAGGAGGAATACACGCCGCCCAAGGTT
CCCAAACTACATTATTAATACAAGCCTTAATTAGATCAAGTGATCCCGTCAGTGATAAAAATAA
TAAACAAATAATATGTTAGGTTTTTTTATTTTTTTATTTTTATAAAAAGAATATTGCATTAAAC
CTGTAGTTAATTTATTTATATATAAGCTTTAATGCAACAGAGAGATTTGTTGCTAAAATTTTGT
AAGGAGCTTAGATTATTATGCCCCTCTTTTTTCATAGGGTGAGAGGGGTCCTCCTTGTAGTAGG
TTTCTAGAATTCTAAATAGTCACTTAATCAAGTAAATTATAGTTCAAATAAGTGAAATGGATGT
TTAATTAGGCAAAAATCAGATCTGTAGGACAGAAATTTCTTAATTAGGGACATAATTAATTACG
ATCTTGGCTTTCATAGAACATTATAATATAAATATTTAACTGGGAACCAAAAAAATCTACAAAG
GTGTACTTTACACAGACAAATTTCACAATGTTTTTTCAGAATATATAAGATTTTTCTTAGAGAT
ATAGTAAAGCTCACTTAATAAAAGAGATCACGAGATAAGATCTAGTTGATGATAATAATTATTA
TAATACTTTATTTAACAAAAATTAAAATAATTTTAATTATTATGATAATTATAAAAATATTTAT
AATAACATCTTTCATAAATTAACTCTAAGTTAATTTACACGGTTGTGGTTATGATTATTTAAAA
ATTAAACAAAGATTAACAAATTTATAATTATAATTAATGAAGTTGTAAAATTTAATTAGAATAA
TCTCAACTACAGTATCAAACAGTCGACGTTGTTGGTGGACGTTCCCAGTAGAGAGAAAGAGAGG
GAGAGAGAGAGAGGGAGGTGGGCGGGGGAAGAGAGAGAAAGCGGAACCCGGACAAACAACTACA
AAGCTCC
-Exemplary Arabidopsis thaliana quick response stress responsive
promoter (rrAtZat12)
SEQ ID NO: 29
AAGGTATAACGAAGATTTGTTCCGCGTGGAAAAGGCATTAAAAGTGCCACGTCACTCTCTCTTT
TTATTTTATGATTTTCGTATCTCTTCTTCTACTTGCTTCCCACGTTTCCATCAAGTTTCCGTAC
ATATCTTCTTGTTATCTGATCCACGCGATCTTTCAACGCGTACTTTTCACGTATTTGTGTTGTC
ATGCCTTTGCTGGGATTGTGTTAGATGCTCATTGCTGACGGTAGTTTTTAGAGAACATTCTAGA
AAGAAACTATTTTTCTAACAAAACCACGAACTTTGTTTTCTAGTTATTCCACTTTCTAGAATAC
ACCTGACCAAATTAGAATTCTAGAAATGAATTTTAAATAAACCAAAACACCTAAACGAAAAGCA
AACCATAGGTTTTTGGTTTTAACATATTTCAAATTCATAAAAGTGAAACCAACCTACACCATAT
TAACCAATATTTATTAGAGTTTTTATATGTTTTATGATATTGTTCAAAACTTCAAAAGAGATTT
ATTCATATAACATACCTATACCATACCAATGAATATTAAAATTATGAATTAGTATCCTTATATT
ATATGAAGTCAATCAAAAAACTTAGAAGCATTTCAAACGGAATCAAACCATTCATATATGAAGT
ATTATTATTATATCTAGAAGGTGTTGATTTTAAACTATTCCGTATAATATATCTAGAAGACGGC
TCCGCGCGTGGGGAATGCATCAAACTCAGAGAGTTTAATAGCTTTTTTTGGTTGACGTCAACTA
CTCAAAAGAGTTTAGTTTTTGATGTGTATATATCCAAATAAAATATCTTTAAAAAGAAAATAAT
AATAATAAATGGTTTCGAGAAAACACGAGGAAGATTCTCATCCAACCGAAACGACTCTTTCGTT
TTTAGTAGTCTCTTAAGCTACGCGGTGTCGCAAATCGTGACCACATAACCCGTTT
-Exemplary Epipremnum aureum auxin signaling responsive promoter
(rrEaPin12)
SEQ ID NO: 30
GCTACTTCTTTCAGCCACGCACTGCGCTTCAAAACTTCCACGGTACCATAGTCGAGTTTGACGA
GAAAATGTCGAACTTGTGGAGAGGAAGAGAAAGTGATCCCATGAGAATTCAGAATAAATCCAAG
TAGCAGATGAACAGTACTCGTATTGATGCGCTACGTAACGTATAATACCTGGCGAAAACCATAA
AACCCAAGAGAGCGAATCTTAAGAAGTACTGTTGTTTTTTTTTCTGGGGACACGGTGAGAAGAG
AAGCCTAGCGTTCTCCCCCAAACAGAGTTCTCTCTCCTCCCTCCCCTCCTGTCTAAGTTCTAAA
AAGGTGGCGTGGTCGGGCACATTGCTTCGTCTCTTGCTTCCCGTTCCTGAACCCATTTAAAGCA
GGTGTTGCTTTGTTGTCTGCCTACAGAGCTCCACAAAATAGTAAGCAGATACACAACAACACGT
ACGCCATCGCCATAACTCTCCTTCGCCTCTCCCAGTTGCTGGTTACATCTGTTCTACTACGAGC
ACCTGTCCCCCATTTTCTTTCCCTCCTCTCTGCTTTTTCCCTGTTTCGCGCTCTGTCACCGCTT
CTCCCTTCTCTTTCCCCCTCTGCACTGATGGTTAACGTGCTTAAAATCACTTCAGTIGTCCTCT
TCTAATAAGCAGGGTTCTTCATTGAGAAGAATCTCCACAGGTAAGCAAACATCACCTCGTTAGG
CTTCTCATTCCACTTCTTCACAAAGGGTCCACCGCAAACCCAGATAGCAAGCCCTGCTTCGTCG
TTTGCCCCTGTTCCATTTCCATTTCCACCCGGGGTCACTCTCAGTCATGGTTTCCCGGGGGAAG
CAGTGAGCTGCTTTGTTCTTACTGAAGCCAGGCACACAGGGCCTTCCACCACCGCCACCGTTCT
CCCTCGTTCCCTGCATCAGAAGAGCCACGTGGTGTTCTTGCAGGAT

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter). In some embodiments, regulatory and/or control sequences impart tissue-specific gene expression capabilities. In some cases, tissue-specific regulatory and/or control sequences bind tissue-specific transcription factors that induce transcription in a tissue-specific manner. In some embodiments, tissue specific promoters may comprise leaf specific promoters, petiole specific promoters, and/or stem specific promoters.

In certain embodiments, a vasculature specific promoter may comprise but is not limited to: a Rice tungro bacilliform virus promoter, an Agrobacterium rhizogenes promoter, an Oryza sativa sucrose synthase I (RSs1) gene promoter, an Arabidopsis thaliana sucrose-H+ symporter gene promoter, an Arabidopsis thaliana 5-methylthioadenosine nucleosidase 1 gene promoter, a Cucumis melo galactinol synthase gene promoter, or a combination of any characteristic portion of any characteristic portion of any one or more of these promoters.

Exemplary Rice tungro bacilliform virus promoter (RTBV)
SEQ ID NO: 31
AGTAGTAATATTTAATGAGCTTGAAGGAGGATATCAACTCTCTCCAAGGTTTATTGGACACCTT
TATGCTCATGGTTTTATTAAACAAATAAACTTCACAACCAAGGTTCCTGAAGGGCTACCGCCAA
TCATAGCGGAAAAACTTCAAGACTATAAGTTCCCTGGATCAAATACCGTCTTAATAGAACGAGA
GATTCCTCGCTGGAACTTCAATGAAATGAAAAGAGAAACACAGATGAGGACCAACTTATATATC
TTCAAGAATTATCGCTGTTTCTATGGCTATTCACCATTAAGGCCATACGAACCTATAACTCCTG
AAGAATTTGGGTTTGATTACTACAGTTGGGAAAATATGGTTGATGAAGATGAAGGAGAAGTIGT
ATACATCTCCAAGTATACTAAGATTATCAAAGTCACTAAAGAGCATGCATGGGCTTGGCCAGAA
CATGATGGAGACACAATGTCCTGCACCACATCAATAGAAGATGAATGGATCCATCGTATGGACA
ATGCTTAAAGAAGCTTTATCAAAAGCAACTTTAAGTACGAATCAATAAAGAAGGACCAGAAGAT
ATAAAGCGGGAACATCTTCACATGCTACCACATGGCTAGCATCTTTACTTTAGCATCTCTATTA
TTGTAAGAGTGTATAATGACCAGTGTGCCCCTGGACTCCAGTATATAAGGAGCACCAGAGTAGT
GTAATAGATCATCGATCAAGCAAGCGAGAGCTCAAACTTCTAAGAGAGCAA
Exemplary Agrobacterium rhizogenes promoter (RolC)
SEQ ID NO: 32
AAAGTTGGCCCGCTATTGGATTTCGCGAAAGCGGCATTGGCAAACGTGAAGATTGCTGCATTCA
AGATACTTTTTCTATTTTCTGGTTAAGATGTAAAGTATTGCCACAATCATATTAATTACTAACA
TTGTATATGTAATATAGTGCGGAGATTATCTATGCCAAAATGATGTATTAATAATAGCAATAAT
AATATGTGTTAATCTTTTTCAATCGGGAATACGTTTAAGCGATTATCGTGTTGAATAAATTATT
CCAAAAGGAAATACATGGTTTTGGAGAACCTGCTATAGATATATGCCAAATTTACACTAGTTTA
GTGGGTGCAAAACTATTATCTCTGTTTCTGAGTTTAATAAAAAATAAATAAGCAGGGCGAATAG
CAGTTAGCCTAAGAAGGAATGGTGGCCATGTACGTGCTTTTAAGAGACGCTATAATAAATTGCC
AGCTGTGTTGCTTTGGTGCCGACAGGCCTAACGTGGGGTTTAGCTTGACAAAGTAGCGCCTTTC
CGCAGCATAAATAAAGGTAGGCGGGTGCGTCCCATTATTAAAGGAAAAAGCAAAAGCTGAGATT
CCATAGACCACAAACCACCATTATTGGAGGACAGAACCTATTCCCTCACGTGGGTCGCTAGCTT
TAAACCTAATAAGTAAAAACAATTAAAAGCAGGCAGGTGTCCCTTCTATATTCGCACAACGAGG
CGACGTGGAGCATCGACAGCCGCATCCATTAATTAATAAATTTGTGGACCTATACCTAACTCAA
ATATTTTTATTATTTGCTCCAATACGCTAAGAGCTCTGGATTATAAATAGTTTGAATGCTTCGA
GTTATGGGTACAAGCAACCTGTTTCCTACTTTGTTAAC
Exemplary Oryza sativa sucrose synthase I gene promoter (RSs1)
SEQ ID NO: 33
CAATCCACCAAATCAAACCGTGAGATTTTTGCAGAGGCAAAACAAGAAAAGCATCTGCTTTATT
TCCCTCTTGCTTTCTTTTCATCCCCAACCAGTCCTTTTTTCTTCTGTTTATTTGTAGAAGTCTA
CCACCTGCAGTCTATTATTCTACAGAGAAAAAGATTGAACCTTTTTTTCTCCAAAGCTGACAAT
GGTGCCGGCATATGCTAATAGGATACTCCCTTCGTCTAGTCCCTTCGTCTAGGAAAAAACCAAC
CCACTACAATTTTGAATATATATTTATTCAGATTTGTTATGCTTCCTACTCCTTCTCAGTTATG
GTGAGATATTTCATAGTATAATAAATTTGGACATATATTTGTCCAAATTCATCGCATTATGAAA
TGTCTCGTTCGATCTAGGTTGTTATATTATGAGACGGAGAGAGTAGATTCGGTTATTTTTGGAC
AGAGAAAGTACTCGCCTGTGCTAGTGACATGATTAGTGACACCATCAGATTAAAAAAAACATAT
GTTTTGATTAAAAAAATGGGGAATTTGGGGGGAGCAATAATTTGGGGTTATCCATTGCTGTTTC
ATCATGTCAGCTGAAAGGCCCTACCACTAAACCAATATCTGTACTATTCTACCACCTATCAGAA
TTCAGAGCACTGGGGTTTTGCAACTATTTATTGGTCCTTCTGGATCTCGGAGAAACCCTCCATT
CGTTTGCTCGTCTCTGACCACCATTGGGTATGTTGCTTCCATTGCCAAACTGTTCCCTTTTACC
CATAGGCTGATTGATCTTGGCTGTGTGATTTTTTGCTTGGGTTTTTGAGCTGATTCAGCGGCGC
TTGCAGCCTCTTGATCGTGGTCTTGGCTCGCCCATTTCTTGCGATTCTTTGGTGGGTCGTCAGC
TGAATCTTGCAGGAGTTTTTGCTGACATGTTCTTGGGTTTACTGCTTTCGGTAAATCTGAACCA
AGAGGGGGGTTTCTGCTGCAGTTTAGTGGGTTTACTATGAGCGGATTCGGGGTTTCGAGGAAAA
CCGGCAAAAAACCTCAAATCCTCGACCTTTAGTTTTGCTGCCACGTTGCTCCGCCCCATTGCAG
AGTTCTTTTTGCCCCCAAATTTTTTTTTACTTGGTGCAGTAAGAATCGCGCCTCAGTGATTTTC
TCGACTCGTAGTCCGTTGATACTGTGTCTTGCTTATCACTTGTTCTGCTTAATCTTTTTTGCTT
CCTGAGGAATGTCTTGGTGCCTGTCGGTGGATGGCGAACCAAAAATGAAGGGTTTTTTTTTTTG
AACTGAGAAAAATCTTTGGGTTTTTGGTTGGATTCTTTCATGGAGTCGCGACCTTCCGTATTCT
TCTCTTTGATCTCCCCGCTTGCGGATTCATAATATCCGGAACTTCATGTTGGCTCTGCTTAATC
TGTAGCCAAATCTTCATATCTCCAGGGATCTTTCGCTCTGTCCTATCGGATTTAGGAATTAGGA
TCTAACTGGTGCTAATACTAAAGGGTAATTTGGAACCATGCCATTATAATTTTGCAAAGTTTGA
GATATGCCATCGGTATCTCAATGATACTTACTAAAACCCAACAAATCCATTTGATAAAGCTGGT
TCTTTTATCCCTTTGAAAACATTGTCAGAGTATATTGGTTCAGGTTGATTTATTTTGAATCAGT
ACTCGCACTCTGCTTCGTAAACCATAGATGCTTTCAGTTGTGTAGATGAAACAGCTGTTTTTAG
TTATGTTTTGATCTTCCAATGCTTTTGTGTGATGTTATTAGTGTTGATTTAGCATGGCTTTCCT
GTTCAGAGATAGTCTTGCAATGCTTAGTGATGGCTGTTGACTAATTATTCTTGTGCAAGTGAGT
GGTTTTGGTACGTGTTGCTAAGTGTAACCTTTCTTTGCAGTTCCTGAAATTGAGTCATG
Exemplary Arabidopsis thaliana sucrose-H+ symporter gene promoter
(AtSUC2)
SEQ ID NO: 34
AGCTTGCAAAATAGCACACCATTTATGTTTATATTTTCAAATTATTTATTACATTTCAATATTT
CATAAGTGTGATTTTTTTTTTTTTTGTCAATTTCATAAGTGTGATTTGTCATTTGTATTAAACA
ATTGTATCGCGCAGTACAAATAAACAGTGGGAGAGGTGAAAATGCAGTTATAAAACTGTCCAAT
AATTTACTAACACATTTAAATATCTAAAAAGAGTGTTTCAAAAAAAATTCTTTTGAAATAAGAA
AAGTGATAGATATTTTTACGCTTTCGTCTGAAAATAAAACAATAATAGTTTATTAGAAAAATGT
TATCACCGAAAATTATTCTAGTGCCACTTGCTCGGATCGAAATTCGAAAGTTATATTCTTTCTC
TTTACCTAATATAAAAATCACAAGAAAAATCAATCCGAATATATCTATCAACATAGTATATGCC
CTTACATATTGTTTCTGACTTTTCTCTATCCGAATTTCTCGCTTCATGGTTTTTTTTTAACATA
TTCTCATTTAATTTTCATTACTATTATATAACTAAAAGATGGAAATAAAATAAAGTGTCTTTGA
GAATCGAACGTCCATATCAGTAAGATAGTTTGTGTGAAGGTAAAATCTAAAAGATTTAAGTTCC
AAAAACAGAAAATAATATATTACGCTAGAAAAGAAGAAAATAATTAAATACAAAACAGAAAAAA
ATAATATACGACAGACACGTGTCACGAAGATACCCTACGCTATAGACACAGCTCTGTTTTCTCT
TTTCTATGCCTCAAGGCTCTCTTAACTTCACTGTCTCCTCTTCGGATAATCCTATCCTTCTCTT
CCTATAAATACCTCTCCACTCTTCCTCTTCCTCCACCACTACAACCACCGCAACAACCACCAAA
AACCCTCTCAAAGAAATTTCTTTTTTTTCTTACTTTCTTGGTTTGTCAAAG
Exemplary Arabidopsis thaliana 5-methylthioadenosine nucleosidase 1
gene promoter (AtMTN1)
SEQ ID NO: 35
CAGCGAAAACACCTTTGATGGGAGCGGTATCAGGAGGCTCTTGTCCAATAAATTCGAATTCGAT
AAGGTAAACTACCATACATATATATGTTATCTAGCTTTTATGCTAAAGGAAAACTTTTTAAATG
ATGGTAACGAGTGATGATGATCCGGAACGGTTTGGTCGCAGGCACTAAACGTTGCCATGGAGAC
GATTCCAAAAGACCGTCAGGGTAAGGTGTCTAAAGGATATCTACGAGCTGTGCTTGACACTGTT
GCACCATCGGCCACTTTACCACCAATAGGCGCTGTGTCCCAGGTAAATAATGCCCCGTCTAAAT
TATTTTGTCTTTTAAATTGTTTATTTTGCCTTTGAATTTACATGTTACAATTATTTGTTAAACA
AATGAAACCAGAATTAGTGTTTTAATCAAAAATTATTAGTGAATTTTTATTTTTATTTTTTGAA
CGGCATTGATTAGTTAAGTTTGTTTTTGTTTATAAGATGGATAATATGATAATGGAAGCGTTGA
AGATGGTGAATGGAGATGATGGAAATGTGGTGAAGGAAGAAGAGTTTAAGAAAACAATGGCAGA
GATATTGGGGAGTATAATGTTGCAGCTCGAGGGTAGTCCCATATCGGTTTCCTCTAACTCGGTG
GTTCACGAGCCGCTCACCTCGGCTACCTTTCTGCCGTCAACTTCGACTGATACAGAGGAGCCTT
CAAACTAATCATAGAAGGGAATAAGCAGCACTAGCAGCAACAAATGTTATATGGTTTTGACTTT
TGAGTGTTTACCCCCAAAAGTTTTAGATTAATGAGGAAAACCGTCTTTACTTTCAGATGTATAA
AATTGAAAGTTTGGGGTTTCCTCTTGTTGGTGTGGTGATTCTACTCATGCCTTTTTTTTTTTTT
TCTAATGACCATGGGATGCAATGTTTACTCTGTTTTTTAATTTCGTTAAAATTTGTTTACGTTT
ATGATGCTTGAATGGCTATGATGAAACATTTGAGTTATCTTTAAAAGTGTGAAATAAATATTCT
GAAGTTAATTGAAGAATTTGAAAATTTGATTACAAGAGCTTGGCTAAAACTACAAGGAGACCAG
ATTAGTACAAAAACTTAGCTAAATTTAATTAATTACGGTCATTAGCACAAAAAAATAATTTGTT
TTTATTATATTATTATTGGTAAGTGGAAACACAAAAGAGGACCAAAAGGTCCAAAAACGAATAA
ACTGTATCTCTCATTCGCCGGAGTTTCCAGCCGTTTCTTTCCGATTCTCGGATTTTTCCTGGGA
ATCAAACGCATCGCCGAGAATCGGAAGAGAGGGATAAGGTT
Exemplary Cucumis melo galactinol synthase gene promoter (CmGAS1)
SEQ ID NO: 36
TCTAGATGACTTGGATTAATTCTCTAACAAGAATTTAGTTTAATTGACATTTGTATGTTTGAGG
ACTAAGAGGACTTTAGTTTTAATTTCTAATCTAATTTGTACTAGAAAAGAAAAAAAAAGAGTCG
GATTAATTCTCTACCATTGAGTGGAGGATACTTGGATGCAGTTCAAGTTCTCATCTCTCCAATT
TGTCACGTGACAGCGGATGATTAAGCATATGAGTAGGCTGCAAAAGATTATAGACGTAGAAGAT
GATACCCAATACAAAGGCGTAACTTTTCCCGGATGACTTTTATACTCTTTACAAAATTGGAAGT
CCTATTCTATCTACATCTTAATTTCCAGTTGTTATAATGAAGAATAGTCTGAAAATGATATCAA
TTTTTTCTTTCTCAATACCATTCAATTACGTTAAGATTATTAGGAGCTGCCATTATTATTATTA
TTATTGTTGTTGTTATTATTATTATTATGCAACCAAGTTTGATTTGAAATTGTTTGCCAAATTT
TACTCCAATTTGATGTTGTTTAATTACTTTAGATGGTATAATAAGAATGAAGTTGAATTTAAAG
AAAAGAAACAAAGCTTGAAAGAATGGAATACTTAGGTGTAGAAGAAGACAACGTATTTATAACG
TCGTATAGTGTAAATAAAAATGCACACATTTGGATGCCCTTTATGCTTCTTAGAGGTCAGACTT
TCCCACAAAGGCTAAGGTGATTCAATCGTGTGGGACATCTTGTTCTCCCATTTGATTCTCGTTT
TCATTAGACCAAAATTAACAAAAAAATAGTAATAATTCTATTCTTTTTAAAGTTTGTGATATTA
CGGTTTATCCTTTGTTAAAAAAGTTTATCTTTGAATGTAAGAATTTGATAGAATGTTGAATGAA
AATTAAGATTTTGAAAAGTTTTGCTGAATTTCAAATAATATAACTCTCTAACTTTGGTTTAGGA
AAATTAAGTGATGACAATTATCTCTATTAGAATTAGTATTATAAGTGATATTTGAGTTATGCAC
TTGACTTGGTCGTGTTGGTAAATTCTTTGGATACAGAACAAAAGAAGTTGCATGCCAAGAAAGA
TTTCTAATAGATATGGTGAGATATGTGGCCGTTGGCTCTATTGGATTGGTGGTATGTTCCAGAG
AAGAGGAGTGCGTATGGATACGACCTAGGTGGATAAATGATTATATGAGGAGATGGTAATTTTA
TGAAATGTGTTAGAGCTTTGATGTTAATATATATTTTTTAAGTGTGTTTTGTGATCGATGGTAT
TAGATGAGTTCCTTATTAAACATGTTTTCTTGGTTTTTCTCGAGGTGGGGTTCTCAACACTTGG
TAACATGCATCATGTCCACGAGATGTTCTTCATCTTATCTCTTGTAATATTATATATGATATCT
CACACAATACAGGTTCGTCTGAAAAATCTTTCTTTATTTGAAATTTTTTAGGTATTTATTCTTG
AGGATTTTTTTATTCTTAAGTAAAGTGTTCATGATTTGAAGTTAGAAATATAGGAGTTATTTTT
AAGAGAGAGTCTCACACTCAAAGGGAGTCTAAATATCTTTTTTACTAATTTAGGTTGTGTAATA
ACCTTGTATTTATCGATAAGTATCACGATGTAATCATTTAACTATCTATTAACGAAAATCTTTT
TTAGGACACGTTGCCTCCTAGATAGATGCAAGTTGTATTGCAAAACTTGTACTCTGTTTTTTAG
TTTTTTACATGTTTTACTTTAGAACTAAACCTAAGTTATGTTATGTGTCAAATAAACTTCTTTA
AAATAATATTAAAACTTCTCAAAATAATAGGAAAAAAAAGAAAAATTTCAAATTTAATATATAT
ATATATATATTGTAATATTAGCTTTCATTATCATTGAATTAAAAATTGCATATACAAGAATCGA
ATAATGTGGAGAAAGTAGTTTTCCTTTTTCAACTTTGTGTAGAGGCTAAGTCTCTAAAATATTG
GCTTCGACTTTGTACTTTTGGATCCGCCACCACAATCAGACAAACTTCCATTTGATCATTACCT
TTATCGAATCAAATTCTTTCCCTTCCAATCTGTCACAATTTTGAACATACCATCCACCTTCTGA
TTTTTTGATTCTAAATAAACCTTATTAGCAGAGATTTTTAAAATTAGTATTAAATTATACCAAA
TACCCTAATGAACTTTTTCAATAGTTTTTCTATTTTATTTTTTTTTTCTTTTGTGTGTATGAGT
TTTTTCACCACCATTAGAAAACACATTTGAAATATACAGAACCAAATTGTTTAATTTGAATTGG
TTTTCCATACCATTTTTACAAAATACATAGTATAACCAAAAGAACTATAGTTTTAAGTAGTGTA
TAATAGTTTAATTTTAAAGACAAAGAACTAAACAATAATCATTATCAAAAACACTACCTTAAAA
CAGAATTGAAATCAAATCCATTTGTTTAGGAATATATATATATATATATATATATATAATATAG
TATCATAATATATAAAAAAAATGTCAAAATCTGAGATTCTTTGATCCTCCCTAAATTGTCCATT
TTTGTCTTGCCTACAAACTTGCAAAAAAGAAAAAAAAAAAGGTTCATAGATAGAAATGACCCAT
AATTGAATCATAAAGCAATAAGGATATACAAAATTATTATATCCAAGAGGGATGAGAGATAATC
TTAAAGGTGCAAAAGAATCTTCTTATTGATGGAAGAAGAGAATACAAACTCTTCCAACTTTTGA
TCAAAATGCCCATAATGCCCTCCATCTCACCTTAAAGATAGGATATTCCAAGTCATATTCATCC
CACCAATACCAATATCTAAAATAATAAGTAACAAATAATTACAATTACAAATATAAAGTGCATA
GAAATTAAACTTAGGGGTATCTATAAACTTAAAACAATGTTCCCCAAGGCTCTATAAATAGCCT
CCTTCCCATCCCTTCACAACTCAAGCTTGAAGGACTAAAACAAGAACTTGTAAGCTTGCCCTTC
TTATTAAGTCCTTCTTGCCTCCCTTCCTTCGGAGAGAAAAAACTTTTGTTGTTTCAAAAGCACC
AAAGTCAATATGTCTCCTGCA

In certain embodiments, a leaf specific promoter may comprise but is not limited to: an Epipremnum aureum metallothionein promoter, an Epipremnum aureum ribulose bisphosphate carboxylase/oxygenase activase 2 promoter, certain Epipremnum aureum hypothetical protein promoters (e.g., hypothetical protein AQUCO_03600155v1), an Epipremnum aureum carbonic anhydrase 2-like isoform X1 promoter, or a combination of any characteristic portion of any one or more of these promoters.

Exemplary Epipremnum aureum (rrEaLeaf1 or P18)
SEQ ID NO: 37
AGCTACGCTCTTTGTCCACAATGTGACAAGGAATGAGAACGAGTCAGCAGTAGATCATCTGGCG
CGCTCTCTGATTGGTGCGTTCACCTCCCGTACCCATGGGCACGCACCCGAGCAGGACCGGGCAC
CCCCAGTGAGCCCCTCACATCCATTTCCTGCCCTGTCGTGGAGTGCAGTCTCTTCGACGTCCCC
GCCTTATAATTAATTACCTGTGCGTATTCGTCCGCACGCTACTGTGCAACGATTCCACCATAGG
ATATATGAGGGGCTTATGCTTATCATATGGAGTTCAAATTTTCTTTTTTATTTTTTTTTATTTT
TTAATTTTTTTATTCATAGTTCTAGTTGGATTTTTGATATTAGAGCAGGTCTTTTTACAAAGAT
GCTATTTTTGTGAATTAAATTTACGAATTTGTCATCTTTATTTTAATATAATCATAAAAATATG
TATGATAATATAACATAAATTCATGTGCAACAATGACATATTTGTCAAAAAAAAATTATTAAAA
TAATGATTATGGAAGAGGAGAAGATATAGAATTAAAAAATCAGATAGGACAAGAGAAGAAGATA
AATCAGAACTGGCCATCCTTTGAATTCAAGTTTGTTTTTAGTTTATTTAATTTTTAATTAATTT
TATGTGGTCCGACCACAGAAAAAGAACAACCCTAAATTTAGCCTTCAATACATTACTGTGGTGC
GAGGAAGCTGCGTCCCCATATGCCCATGGCGTGTGGAGCTGGTACGACTGCTTCTGTCTCGACG
TGCGTTCCCCCCGGAAGAAAAAGAGAAGGAAGTGACGTGAGAGGTCCAGAGGCAGCCGACCTTC
TCCTCCATTATCGGGAGAGATTCCTCTCGGGACTCCCACTCGCAAGAGCCCTCTC
Exemplary Epipremnum aureum ribulose bisphosphate
carboxylase/oxygenase activase 2 promoter (rrEaLeaf2)
SEQ ID NO: 38
TTGTTCAGAAAGGAACCCCCTAGTTTGTAATTGGAGGTCATAAGAGGTACTTTCAGTCCTCAAA
ATTTATCATTTCTTAATGAAATTTTTAATTTTAAAAGATTTATTCTTTTTAATAATTTTTAGGT
TGAGATCAAGTAAATTTAGAAGATGATTTTGACAACGATTTTTTTGAAGTAGATAATCAAAATT
AGGAGTTTTAAGAATGATAATAATTATTATTTTAATAAAAATTTAAACTCACCTTCTATAAACA
GATGTCTCTCATTGTACCAAAAATTTTAGATTTACATATTATTATAAAAATATCTTTTCATTTT
ATAATTTATAAAAATATTTTTTAAAATTAATTTATTTCAAAATCTATCATGAGCTGTCTTAAGA
TAAGAGTTGCATAATTATAATTATTTTTTAATTGTAATAAATAAATATCCATACTACCCTCATG
TTAAAAAAATATATATATATATATATAAAATCATCCCTCCCCCTCTCTCTCTCCTCGTCTCTTA
TGTTTCTGAATCACATTTTTTTAAAAATATTAATTAAAAATAAAATATTTTTAAATGTTTTAAG
TATAATAATATCTAATTAAATTTTTTGAAAACATTTTTTAAATTATTTTATAAATGATAAAAGA
GATCTTTTTGTAGTGCCAGCTCGTAACAAGGTATATTTACGAATAACCCTTCCTTTTATTGCAG
ACACCTCGGCTGAGAGTACGCAGTAGATGACGGGTCCCACTTTTTTTCCCCACGCTCCAAATAG
CTCCAACGTCGTCAGGACACGACTTATCTGAACAGAAGTTATCCGCCCTGATTGCGCCACGTGT
TCCGGCCCAATCCCCACTGTGTGGCCACAGGACCCTCCGCTCTCCCCCTCTCCTCCCCTCCCCT
CCGCCAGCCAGAGGGAAAAGGAACAGAACAGGGCGATCTCCAGAACCTCCGCAGGCCGCTTTAT
ATATAGTTCGCCCTACCCCACCGCCTCCGGCCAACGCTGCTACGAGGAGCTGAGCTTTTGGTGG
AAGCGGCGATCCCCCCCTTCCGCCTTCTAGGTCTTCCGGGTCCC
Exemplary Epipremnum aureum hypothetical protein
AQUCO_03600155v1 promoter (rrEaLeaf3)
SEQ ID NO: 39
GTGCGATCCCTCTTTCCCTCCACAAATTAATAAAGCCTGATTTGGGTTTTGATCACAGAAGATC
TGTGTTGCTTGATCGATGTGTTGATAAAGACTAAAAAGAAAAAGAAATCCTCGATCTATTAATT
TAATTTTTAAACAATAAATTTACCTATTCTCTTTCCATTCCCTTCAGTCTTCATGGTTTCATTA
ATGGCGTTATATGCCCTTGTGAGAGATTTAATTGCGTAACTATCTCTTTTAGATTTGCATCTTC
ACGCGCATGTCATCCTCATGCGGCAATGTACCTATCTATCCCTCCCGTGAGGGTATATATACGA
TTAAAAGTATCATCAAGATATTTTTAAAATTTACAGCTATACACCTCTTAATGATATAATGGCA
CACACGTTTGAAGGAAGAGAGTGTATACACACGAATGTAAATTTAGAAAGGATATTCATGCAAG
TGGGACTCTAATAGACATGTATGGAAAATGTCTGTTTTTTTTTAACCCATATCCAATTCACTCG
AGTATAAATGAAGGTGATAATTATTTGCATGTGCTTGGCCTTTTTAATGTAAATTTGGTTTATA
CCAGTGGCATGTATTCAAACTTCCTTTATTTTTCGGTCTGCATCCATCTCCCTCTCTCTGGTGT
CTTCTTCTTCACGCAGCCAGAGGTTAAGGGAGTTGCGTGTGCAAGTGCAACTGGGCAACAGTGC
AAGCATAGCCAAAGGGAAGAAGAAAGAAGAGGAATTGACACGAGAGGTGGAGGGGTAGCCCCCC
TCCTTCCCCACCATAATTGAGATTCCTTTGGAAGCTTCCTCCATGGAGGCGTGTGCCCATCACA
CACAGGGGCCCTCCCCTCCCCTCCTCTCCTTGTGCCGTGTGCGTCCCTCTGCCATCCCCCCCTG
GGGCCTATAAATATCGTCGCAGGGTGGAAGCCCCTCCACCATAGCTGGAGCTGACCCCTGAGCT
GAGAGATATATAGCAGAAGCTCTCTTTGATCATCTCTAGAGGCTCCCCTCTGC
Exemplary Epipremnum aureum carbonic anhydrase 2-like isoform X1
promoter (rrEaLeaf4)
SEQ ID NO: 40
CGCACGTAGCCTTCGTTACTCATCTTGTTGTTCGTCTAATTTGGAGAGATGGTTTCAAGCATTT
GACAATCCAAGGAGACAAAGTCATTAGTATTAATGTTTCTCTGTTAATTAATTGTCTCCCTGAT
ATCCTGTCTCAAGTATGTTTATGTGTGTGTGTGTGTGTAAATATAAATATAAAGAACAATATGT
GATAAAGGATAACCATTCTGCATGGTGGATTTGTCTTCATTAATTAATATAGTTCTTTCTTTCC
ATCATTTGATTTCATTTCATACACTAGTACTTTGGTACCATGTTTATTTTTCAAGGTTTATCGA
ACAGGAATTATTCAGAAGATATACCAAAAATCGATTGGATTCATTCTCTATTCAGACTGTTAAT
TGTTAACCATCGATTTAAACATGTCATCTTAAGGGAAATTAAGAAACTAGATTGTGTTTACGTT
TTCCACACTGTTAGACCTTCTATAGTATCTTCATTGTTCTCGAGTCGATTGGTAGTATTGGAAC
GAACTAGCATGCATGTGTGGAACACCCCCTCTTATATACTGCAAAAAATGAAAAAGAAAAGAAA
ATGGACCATCACTTTGATTTTTTAGGGTTTGGTGGCTTCAAGACACGATGCTTGGCTGGGTGCA
ATTAAACTGTGCCATAAAAATGTACTATGCTATTCAATAATCGATTTCATGAGACATGGTACAT
GTCATATTTCATAAATGACGTGGTACATGCCAAATTTCATAAGTTTTCTTGTCTAGAAACTTAA
TAAATTACTATTCGCATAGAAATCCTGAATTTTTACTATTTCTGATTTCCCCCACCCCCAGAAT
TTTAAGGTTGAAGCTATCAGAAAAACAAGAATTATTATATATAATCCATCTGCAATGCATGAGA
TTAGCGATACACCTGCAACGCCATCACCTATTCCATCCAACGATTACATGACACTGTCATCTCC
AAGCCTTCTCTCTCTCTCTCTCTCTCCCTCTCCCTTATTTGAAGCAGAAGCCATGGITGATCCG
GCTTTCGCTTTCCTTATCCTAACCCACCCCCGTCGCAGAGACTATATATCGAGCCCTCCACCCC
TCCTGGGACGGGTGTGAAAGAGAGCA

In certain embodiments, a petiole specific promoter may comprise but is not limited to: an Epipremnum aureum beta-galactosidase promoter, an Epipremnum aureum vacuolar-processing enzyme promoter, an Epipremnum aureum cathepsin B promoter, an Epipremnum aureum metallothionein-like protein type 2 promoter, or a combination of any characteristic portion of any one or more of these promoters.

Exemplary Epipremnum aureum beta-galactosidase promoter
(rrEaPetiole1)
SEQ ID NO: 41
TTCGATCTCCCCCTCGACTTGAAAAAACTAATAAAAAAATGTAACCTTATATTTTTCCGTAAGT
AAAACGGAAAGTATATTTAATAGAATATAAAAAATCTGTAATTTAATTATTATTCGGATAATAA
GAGAAAGAAGAGGAGGGCAAAATTATGGGAGTTGATGGATGGATGATGCTGCCACGTCAGAACT
CGGACCGGGACGTGGCCGGCCGGGTGGCGCCGGTCCTGCCCGCCCACTCGCTTTCACCCCACGC
CCTTTAAATCCCACCCGGCGCCCCGTTTCCCTCGCCACGGCCATCACCACCAACGGCCTCTCTC
TCTCTCTCTCTCTCTCTCGCGATCTTCACAGCCACTTCTCACTCCATTACGCTCTTGTTTACTC
CTCACTCCCATCTCCTTAAACGCAAGCGACTGCAACCCAAACCACGCTCTTCCATTGGCCTCGT
CCTCCTCTCTCGTATCCCGAAAGCGAGAGAGGACCGGCCAGAGAAAGGGGACAGAAGAAAAAAA
AAAGAGTCGGAGGGAGAAAAAGAGTGGGCCGAGCGAGAGGAGTTGGAGAGAAAATTATACTGAA
GAGCACCCTAAAGCGGGCAAGGAATATTGCTGGGGAGTTGGGAGGAGAGAACAAAACGAGAGAA
GGAAGAAAGAAAGGAAGAGGGAGACGCGCAGTGTTACAAGGAAGATTAGGGGATAAAAAAAGCC
GTTTTCTTCTTCTCTGCTGCTGCGAGGTCGCTGACCGCCTTCCTTAGACTCCTCTGCTGGACGC
ACTACTTCCCATCTTATCTTAGCTTTCTCCAACCTTTAGCTTCTGACACATTAAAGAGGAGGGA
ATATAGAGGAGAAAAAAAAAAGATCGTCGGAAGGAAGAAAGGAAAAAAAAAGATCCAACCAGGT
TTCTGCGGAAG
Exemplary Epipremnum aureum vacuolar-processing enzyme promoter
(rrEaPetiole2)
SEQ ID NO: 42
TGGTTGAAGTGCTAAATTTGGCATTGCCTCAATTTTGTTACTAAGATTTTTGTAATATCAAAAA
TTAATATTATAATTAATTTAACACAAAGTTGAAATAATTCAGATGATCTTGTCAAATTATTAAT
ACTGTTGATGATATTACACTATTTAATAAAAGAACCATATGCCCCATAAAATTAACTCGGCCTT
CACTGAAGAATGATCAAGTGGTCATTATGTAATCATCTGAAACTCAGGGATGATACATACACAT
ACATGTCTAAAACTCCTAGAAACTGTAGTTAATTGCACCCTTTTGCCACTGCATTATTTCATCT
GGTACCAACTGACATGGCATCCCCTGTCCACTTGCTATTGGATCAACACGCCCGACTTCTTACG
TCGCCACGCCGGGGCCCACCTAGATAGGAACTATCTGCTTGATCCCGTCGAATCAGCAGCGTTC
CAAGCCCGCTCCCCCATCGGATAGATATTAACCGTCGGATCAATGGATCCATCGTGGGAACATC
TATCTTCCAATGCCGAACAGCACAACTAACTCCCAACCGCCACCGCTGGCCCACCCACCGATCG
TTGAGCCGGATCAGGATCCTGCGGCCCTCACGTGACCCCCAGAGAACATCGCCTCCTCATAGGC
CGTCGCGTGCGAGGGCTGACGCCCGTCAACACGACCCCCAGGGAAGACGTCACGTCGGCAATTC
CGGAGATTCAAGGCGAGCGCATAGGCCGCGCCAATTAAGCTAAAACCCGAAGAAATCCTTCGAG
CAGAGCAACAGCTCGGCGGGGCCCCACTTTTTCTAACTTTCCCCCGCTCCAGTCTATAAATAGC
GCCCACTTTCCGCCCAGGTTTCCTCGCCATTGACGATTAGAGCACTCGACGGAGGTAAAGCTGC
TTCCCTGGGTGCCCCCCGCACCACCACCAACG
Exemplary Epipremnum aureum cathepsin B promoter (rrEaPetiole3)
SEQ ID NO: 43
CTGAGGAACCCCATTGCAGTTTTACTACGGTCAGATTGGAGGAGAGATCGAGGCGGCACACGTA
ACGGCAAAACGTCACGTTGACGGGGCTCTTATGGTTCCCGTGTTACGTAAACCCCCGGCATTGG
GACCATTGGGACTCACCAAGTCCCGTGTGCGATTGTCTCTCGAGTGGCGTGCCTCATCACTCAA
CACAAGGGCGAGGGGTGCACGGCGCTGTCGTCACCCCTTACGTGAGCACGCGGTATAACGATAA
CGGCATCTACCATCCGACGGGAAGGAACAGCGTCAGATCGTAGCGGGATGGACCGTCACGGCCT
CCTATATATCTGATGAAGCGCCGTCAGATCGGGAGCCCTGGGCCCACAGCATTGGGGTGCAAAC
CAATCAAATGCCACTTCCTCCAATAATGGACACTATGGGTTCCAGCTTCGAAGAAGCGGCAGCT
GGCGCCTCCGTAGCTCTCTCTCTCTCTCTCTCAAACGGCGGCGTCATCTTATCCTATCGCCTTT
TCAGAGCCCGGCTGCGCAAGTAACCGTCCCGTTGATTTAGATCTGGATTTCATTTATTTGCTAC
GTTGAAATCAGGGTCCAATCGCACTGCCATCACCCCCAAACGTCCGGATTCCATTTATGTTATA
CGCTGAATCGAGGTTCAGCCGCGTTGCCATCACCGTCGAAATAGGTACCGCCGCCGCCAAGCTT
CCATATCATCTTCCCCCTCATATCAAATTCTGACCCCTCTCTCTCTCGCCCCCCTTCCTTCCTG
GTCTTGCTACTCCGCTCCGTCCCTCTCCCCGTTTCACCTCTCCACCTGCTGTCTGTAAATGGTG
GGGGTGCTGTTTCGAGCTGAAGGGTGAGGGTGTGGGGGTGCTGTTTGGAGCGGAACGGAGAGGA
TAGGGCACAGATATAGCTAGGGGGAGAGAGAGAGAGAGAACAACGGGG
Exemplary Epipremnum aureum metallothionein-like protein type 2
promoter (rrEaPetiole4)
SEQ ID NO: 44
GTACGCAGGCTGAAAGAAGCCTCTTTATTCAATTGAGAAGTGATAGTAACTATTATCCAATAGA
GTAGGGAGAAGACGTATACATCCTTTTCTATGGCATCGTTTACTTTGTCTGTCCACCATGAATG
TACTCTATAATAAGTAGTAATCAATGAAATGATACCTTAAAAAATTAGATGTTTGTAATGGCCC
CCCCTTAGTAATCTTCCTAGTGACGGATGCACTTTAAAATATTGGAGAAAAAAATGATGGTTGC
AGTACAACAATATCATATTAGGTAAGAAAAATACAAGAGTGTGTGGAGACTTGGTCTACTTTTG
ATGTAAAAAAACTGTAAATATTGATGGGTTGAGTTAGTATTATAAAAAAAGAATAAGTTTGAGT
AATTCCTTTTCACATAGAAACCTTTTAAGTCCCTTTCATATATCAAGCAGCAGACAAGAATTTA
AAATTTTGAGGTCTTCACATGTTGGATGCAGTGCTCTTCTAATTAGCTGTGGCGGCAGGAGTTC
ATGAAAATTAAGAAAAAAATGATATGAAAAATGACAAGATTCCCTACTTCATCCGACAATGCAT
ATGGTCTGGGGCAAATTAGAATACCACACTTCTCTCGTCATTCTGTCATTACTCCTTTTTTTAT
TTTAAAAAACTCACCTCATCATTTATAGTACCGCATGTTAACTCAGGTGTTATTTGATAACGTT
ATCAGCGTTGATTTTATCTTTTAATTTTTATAAAATTTTAAAAAATATATAAATATTACTATCA
AATGAATAAATACTAAATCAGATTTAAAAAATAATTTATAATTATTAGATTAAAAATCACTTTA
ATTCATTTTAATAAAATCTAAGACAATCATAATATTGATATGATTTAAAATTTAATAAGAATAA
CATAACGATAATATTATCAAATGAAGTGTTTCAAAGATCACAAGTTATCCCATGTTCGCAAGAA
GGGTAATATAACTGTTGACGGCACAACTATTGTAGGAGTTTTAAATAAAGATCTATATAACTTG
ACATGACGTGAGGTAGCAGAGACCATCAAGA

In certain embodiments, a stem specific promoter may comprise but is not limited to: an Epipremnum aureum metallothionein promoter, an Epipremnum aureum dormancy-associated protein 1 promoter, an Epipremnum aureum dehydrin COR410-like promoter, an Epipremnum aureum ubiquitin-conjugating enzyme E2 8 promoter, or a combination of any characteristic portion of any one or more of these promoters.

Exemplary Epipremnum aureum metallothionein promoter (rrEaStem1)
SEQ ID NO: 45
CCCGATGAGCACCTCAGATGTCCATTTGATGCTCTTTCGTGAAGTGGATTCTCTTTGACGTACA
CATCTTATAAATATCTATATTCGTCCACACCGCTGTGCAACGATTCCCTATGTGATATATGCTG
CACGGACGGAGAGGGCGGTTGCCTGAAGGAACACATATGCTTATGTGGAGCCCAGTTCTCTTTA
TACTTTTAGTTGGCTTTGATTTAGTTTTTTTTTTTTTTTTTTGAAGTAGGAGCAGATCCTGTGT
TGTTGCAGATTTACTACCTCGGCTGCCACCCATAGAACAAGATCATATTAATCTGTCTCTTGGA
GCTGAAATATGGGGAGCAAAGAAAGGGTATTAGAAAGATTCTTAAAATTAGTAGACCTGTCCTA
AGACACTGGTGATTGAGCAGTGGCATCTGCACTTGTGGACTGTGTGCTTGTGCATGGACGCTGG
CTGGAGAGATCCGCCGACGTGCATGGCGAGGGTGCATCAATAGGACTGGACAAGGGAAGAAGAA
ACATCTGAACTGAGTATCATGTGAAATTAAAACTTTTTAATAATTTTATTTTATTTTAAATTAA
TTTTATGTGGTCCGACCACAAAAAAAACTTACAGAACATTACTGTGGTGTGAAGAAGCTCCGTC
GCCATGCTACTGGCGTGTGGGGTCGGTAAGATTGTCTCTGCCTCGACATGTGTTCCCCCCTACA
GAAGAAAAAGAGAAGAAGTGACTTGAGTGGTCGAGACGCAGCCACCCGTCTCCTCCATTATCGA
GAGGGATTCCTCTGGGGAATCCCACTCGCAAGAGCCCCAGCAATGCCTATAAATACCGGTGGAG
GCGGCCCCTCTCCAGCTCACACAGAGCCGACGTGATAAGCTCCTCCTCTCGCTTCAGCAGTTCT
CTCTTGCCTTCGCCACTTCCCATTATCGCC
Exemplary Epipremnum aureum dormancy-associated protein 1
promoter (rrEaStem2)
SEQ ID NO: 46
TGTGAGTGACCAAGTGTGCTTAAGAGCAACCAAAGACTTTGGTGAGCATCATAGTGCATTATGT
TACCCATCAAATATCATATTGCTCATCAAAAGTTACTCTGTGGATAGCACAACCTACCATGTTA
CTCATATAGAGGTGTCTAGTGAATAACAGGATGTTTTGATGGATAACATAATACATCATACTAC
TTACTAATACATTTAGTTGTTCACAAAGTATCACATTATTTATTCATCAACACATTAAGTTACT
TATGGGCATATAAAATTACTTAAAGTATCCCAATTACTGAGGAAAGATTTAGATGTATAATATT
TTTAACTTATTTCTAGTACAAATGGGGTGCACAAATAGTGAACAGAGTGAGGTCATTTTCTGAC
AATTCCATTGGGTAATTTTTTTTTACTCTCTTTTTTCTTTCAAACTGATTCAAAGAGTTTAATG
GTGACAGAGTCACATATCTAGAAGAATATTATTGGGGGCGGGTGCAATGTTGTTTGCACTACAA
GTCGACGACCGGTCGTCACGTGGATCCCATAGTGGGCCAGGTCCATGCTATGATAAAGCCCATC
AAAGGGCAGATATTTCCGTCGTCACGTGATGGAGGGGGGGCCCAAATCGTCTTCATGCTTATCC
GCTACCTGTCCATACCGCCATCACGTCACTCTCCCACAGCTTTGATCACTTCCGCCCCCTCCCG
CCCAGCTACCCTCGAGACCCGGTATTCGGACGTCTTCTCGGATCCGAAATATCCGCTGTTATCT
CGGGTTTTCTTGTTGGAGTCTCATCCTCCCCTTCACTTGAGACGATCCGGACTCGATCAGAGTG
TTAAAGGATGGGGATGGAGACGTGTGAGTGAGGGCAAAAGGAAACCTACGTACAGGTTGTCTGA
AGGAAACTTTTTCCAGCACTATCCTGCTCTCGTTACCTGTGACTATCCGTTAATTTGGCATCTG
AGCAGAATCTCTTTCTATATATGGAGTTGGCGAGGGCAGCAGCAATAGGGGTGCAGAGCCAGTG
TAGTTGTGGTTGAGAAGGAAG
Exemplary Epipremnum aureum dehydrin COR410-like promoter
(rrEaStem3)
SEQ ID NO: 47
CTGAGGACGCTTCGAGATCCACTGACCATGCCACTTTTTTTTTACGTGAACGAGGCAAGTCGGC
ATTGACGAGCGGGGATGAAAAGGGCCGTGGAGCGAAGGGGACACGCACGCTCATAATACTGTTC
TGTACGGCTTATATAGTATAAACAGATCCAGCGCAGCGCCCGCGCATGTGGCGGGGTATTGGGG
GAGGCGATGGCGCGCGTCTGCTCCCCCGCCGTGAGGCCAAGGACCTCCGGTAGGGGCGCACCGC
TCGCGGTGTATGGCGGCCGTACCGTGGACATGCATGTATGGTGGGCTTTTTTTAAGTTTGCCCC
GGATAAGTGTTACTGTTGTGGACATGCACATGCATACGATGATGGGGTCCGTCTGGGTCCGTTG
CTCTACTCATCCGATGCCACGCAAGCTCTGTAGTAAATGTATGTATATATTCGTGTGAGAAAGA
GGAACGAAAAGGGACAACTAAGCGAAGTCCGATGGCTCATCTTAATGATTAAATTACAAAAAAA
AATTATTTAGATATCTTCGTATCAAGTCTCTAGAGAATAATCTGTCATTTAAAGTTTGAGGTTA
TTTTATGGATATTTCTTTCTCCTTTAATGACTTATAAATATTAGATTTTACTTCTCTCAGTTAT
AAAATCACTCATCATTCCAACTGAGTTATTTATCTAAGATTTGATGACAAGGGGAAGACGATTA
CGATGGGCGCTCTCCAAGCGTTGCTGTGGAATTTCTCGCGGTGAGTGGCGATGACACGTGAAAC
TTTGTCACAACTACTCCAAGAATCCCACTAGCCATTAGCTTGTATGATATTAATACTGAGACTG
GTTATTAACAAACATCTAACACCACCTTTTATTTACCAGACGAGGACGGTAACGGAAAACAGGG
GAATGAAAGCAAGAGAAAGCCGACATCGGACCGACGTTCCTCGAGGCCCGATCTGATCCACTCC
AACCCGCCATCGTCAGCATCACCGTCTCAAATCAAGTCCATTTATCGCCCGCTGCGAAAGGGAA
AGGCAAAGGGTTTGAAAAAAAAAAAGAAAGGCAACGAAAGGGGGACGAAGGTGG
Exemplary Epipremnum aureum ubiquitin-conjugating enzyme E2 8
promoter (rrEaStem4)
SEQ ID NO: 48
ACATGACACTAGGCAGGATCATTCAATACAACTAACTTGAAAGATAATGAAAGAAAATAACAAT
AAGTGATTACAGTGTTAGCATTAATTATTTTTTATTATCTTCATCTTTTGTCCCACTAGTATTA
AATACTTAAAAAATGTTTAAATTATATGCGATCACTAAGATGAGGGGGAGAGGGGGGTATGAGT
AACTAAAAACATCTTTATATTATAAAAAGTAGTGCAATAAATATCACTCTATTTATATGTAAGG
GCAAATGTACAAATAAGAGAGATTCTAGGGGCTGCCTCCACAAAAGTCCCTTAAACTTGAAGAT
CCCTTCTAAGTTTTAAGATTTAACATTCTTTTTGTTGAACTAACGCAATTCCACTGAGGTTTAA
TTCAGATTTTACTTAACTAAATTAAATATTTAAAAAATATTATATTTTAAATTTATAAAAATAT
ATAAATTATTTTAAATATTATATTATTTTTTAAATTATTTATAATAATTTAGATAATCCTCAAC
AAACCATGGTTAGAAGTTCGAAGTTCAAACCTGTGCCCTACCGTTACCACCGTGTGGTTGCCTG
CGACCTGTTCGAACCGGATTCCTCTTTATATATCCTTTAAATATATTAGCGCCGCTCCTCTCTC
TCTCTCTGTCTCTCTCGCCGACGGCAGCCTCTGTCCCCTTCTACGGGTCCTCGAGGAGGGGCGG
GGCGGGCGGAGGGGGTCGGTCGCACGCAGCAGGCAGAAGAGAGAAGCATTCCACCGCGCTCTCT
TCCGCGTCCGTTCCCTCCCTCTCCGCCTCCGTTTGTTCCCTGCTTTCCTCTCAACCCTGACGGT
TTCCTCTCTTCTTTCCCCTCTCTATCTAGGGTTTCGGAGAGATTGGCACGTACCGACCGGGGTT
TCC

### Terminator and Polyadenylation Sequences

In some embodiments, a vector comprises a terminator. The term “terminator” refers to a DNA sequence recognized by enzymes/proteins that can terminate and/or end transcription of a gene or operon. For example, a terminator typically refers to, e.g., a nucleotide sequence in the DNA, that induced the release the newly synthetized transcript RNA from the transcriptional complex. This frees the RNA polymerase and associated factors related to the transcription machinery. Thus, in some embodiments, a vector comprises one of the non-limiting example terminators described herein operably linked to a coding region.

In some embodiments, a terminator can code for a 3′UTR and/or a Polyadenylation signal in the mRNA transcript. In some embodiments, a terminator can be a plant cell terminator, a viral terminator, a chimeric terminator, an engineered terminator, a tissue-specific terminator, or other types of terminator known in the art.

In some embodiments, a terminator is one listed herein as set forth in SEQ ID NOs: 49-55. In some embodiments, a terminator sequence is at least 85%, 90%, 95%, 98% or 99% identical to terminator sequence represented by any one of SEQ ID NOs: 49-55. In some embodiments, a terminator sequence is a characteristic portion of any one of SEQ ID NOs: 49-55.

In some embodiments, a vector provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some embodiments, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

Exemplary Cauliflower Mosaic virus 35S terminator (TerCaMV35S)
SEQ ID NO: 49
AGCTTCTCTAGCTAGAGTCGATCGACAAGCTCGAGTTTCTCCATAATAATGTGTGAGTAGTTCC
CAGATAAGGGAATTAGGGTTCCTATAGGGTTTCGCTCATGTGTTGAGCATATAAGAAACCCTTA
GTATGTATTTGTATTTGTAAAATACTTCTATCAATAAAATTTCTAATTCCTAAAACCAAAATCC
AGTACTAAAATCCAGAT
Exemplary Arabidopsis thaliana Actin 2 terminator (TerAthAct2)
SEQ ID NO: 50
AGCTTGCTCTCAAGATCAAAGGCTTAAAAAGCTGGGGTTTTATGAATGGGATCAAAGTTTCTTT
TTTTCTTTTATATTTGCTTCTCCATTTGTTTGTTTCATTTCCCTTTTTGTTTTCGTTTCTATGA
TGCACTTGTGTGTGACAAACTCTCTGGGTTTTTACTTACGTCTGCGTTTCAAAAAAAAAAACCG
CTTTCGTTTTGCGTTTTAGTCCCATTGTTTTGTAGCTCTGAGTGATCGAATTGATGCCTCTTTA
TTCCTTTTGTTCCCTATAATTTCTTTCAAAACTCAGAAGAAAAACCTTGAAACTCTTTGCAATG
TTAATATAAGTATTGTATAAGATTTTTATTGATTTGGTTATTAGTCTTACTTTTGCTACCTCCA
TCTTCACTTGGAACTGATATTCTGAATAGTTAAAGCGTTACATGTGTTCCATTCACAAATGAAC
TTAAACTAGCACAAAGTCAGATATTTTAAGATCGCACCATTT
Exemplary Solanum lycopersicum Histone H4 terminator (TerSIHisH4)
SEQ ID NO: 51
AGCTTTTATGTTGGTGATATGGTGGTAAATGTAGGGATTTAGTTTACAATTGCGTATGTCTGTG
TTGGATATCTGTAGTGCTGTTCTTATGGCTTAGATCTTGTAATTTCTCATTACAGTATCAATGA
ATAGATATCAGTTTCTAGTGATGACATTGGTTCGTCTTTTAGCTGTTGATTAATTTTTCTTAAT
TGATTCATCCTATTGCAATTCTTCTGAATTTAAATTGTATACTGTGAAATTAAGAAAATTCTTG
AAATTAATGAGAATTTGAGTAATAG
Exemplary Agrobacterium tumefaciens nopaline synthase terminator
(TerNos)
SEQ ID NO: 52
AGCTTCTCTAGCTAGAGTCGATCGACAAGCTCGAGTTTCTCCATAATAATGTGTGAGTAGTTCC
CAGATAAGGGAATTAGGGTTCCTATAGGGTTTCGCTCATGTGTTGAGCATATAAGAAACCCTTA
GTATGTATTTGTATTTGTAAAATACTTCTATCAATAAAATTTCTAATTCCTAAAACCAAAATCC
AGTACTAAAATCCAGAT
Exemplary Agrobacterium tumefaciens octopine synthase terminator
(TerOcs)
SEQ ID NO: 53
AGCTTGTCCTGCTTTAATGAGATATGCGAGAAGCCTATGATCGCATGATATTTGCTTTCAATTC
TGTTGTGCACGTTGTAAAAAACCTGAGCATGTGTAGCTCAGATCCTTACCGCCGGTTTCGGTTC
ATTCTAATGAATATATCACCCGTTACTATCGTATTTTTATGAATAATATTCTCCGTTCAATTTA
CTGATTGTACCCTACTACTTATATGTACAATATTAAAATGAAAACAATATATTGTGCTGAATAG
GTTTATAGCGACATCTATGATAGAGCGCCACAATAACAAACAATTGCGTTTTATTATTACAAAT
CCAATTTTAAAAAAAGCGGCAGAACCGGTCAAACCTAAAAGACTGATTACATAAATCTTATTCA
AATTTCAAAAGTGCCCCAGGGGCTAGTATCTACGACACACCGAGCGGCGAACTAATAACGCTCA
CTGAAGGGAACTCCGGTTCCCCGCCGGCGCGCATGGGTGAGATTCCTTGAAGTTGAGTATTGGC
CGTCCGCTCTACCGAAAGTTACGGGCACCATTCAACCCGGTCCAGCACGGCGGCCGGGTAACCG
ACTTGCTGCCCCGAGAATTATGCAGCATTTTTTTGGTGTATGTGGGCCCCAAATGAAGTGCAGG
TCAAACCTTGACAGTGACGACAAATCGTTGGGCGGGTCCAGGGCGAATTTTGCGACAACATGTC
GAGGCTCAGCAGGACCGCTTGAGACCACGAA
Exemplary Agrobacterium tumefaciens mannopine synthase terminator
(TerMas)
SEQ ID NO: 54
AGCTTGGACTCCCATGTTGGCAAAGGCAACCAAACAAACAATGAATGATCCGCTCCTGCATATG
GGGCGGTTTGAGTATTTCAACTGCCATTTGGGCTGAATTGTAGACATGCTCCTGTCAGAAATTC
CGTGATCTTACTCAATATTCAGTAATCTCGGCCAATATCCTAAATGTGCGTGGCTTTATCTGTC
TTTGTATTGTTTCATCAATTCATGTAACGTTTGCTTTTCTTATGAATTTTCAAATAAATTATC
Exemplary Agrobacterium tumefaciens agropine synthase terminator
(TerAgs)
SEQ ID NO: 55
AGCTTGGACTCCCATGTTGGCAAAGGCAACCAAACAAACAATGAATGATCCGCTCCTGCATATG
GGGCGGTTTGAGTATTTCAACTGCCATTTGGGCTGAATTGTAGACATGCTCCTGTCAGAAATTC
CGTGATCTTACTCAATATTCAGTAATCTCGGCCAATATCCTAAATGTGCGTGGCTTTATCTGTC
TTTGTATTGTTTCATCAATTCATGTAACGTTTGCTTTTCTTATGAATTTTCAAATAAATTATC
Exemplary Epipremnum aureum agropine Histone H3 terminator
(Ter7.1)
SEQ ID NO: 409
GTGGCTCTTCAGTGGACGAAAGGGCCTCGTGATACGCCTATTTTTATAGGTTAATGTCATGATA
ATAATGGTTTCTTAGACGTCAGGTGGCACTTTTCGGGGAAATGTGCGCGGAACCCCTATTTGTT
TATTTTTCTAAATACATTCAAATATGTATCCGCTCATGAGACAATAACCCTGATAAATGCTTCA
ATAATATTGAAAAAGGAAGAGTATGCGCTCACGCAACTGGTCCAGAACCTTGACCGAACGCAGC
GGTGGTAACGGCGCAGTGGCGGTTTTCATGGCTTGTTATGACTGTTTTTTTGGGGTACAGTCTA
TGCCTCGGGCATCCAAGCAGCAAGCGCGTTACGCCGTGGGTCGATGTTTGATGTTATGGAGCAG
CAACGATGTTACGCAGCAGGGCAGTCGCCCTAAAACAAAGTTAAACATCATGAGGGAAGCGGTG
ATCGCCGAAGTATCGACTCAACTATCAGAGGTAGTTGGCGTCATCGAGCGCCATCTCGAACCGA
CGTTGCTGGCCGTACATTTGTACGGCTCCGCAGTGGATGGCGGCCTGAAGCCACACAGCGATAT
TGATTTGCTGGTTACGGTGACCGTAAGGCTTGATGAAACAACGCGGCGAGCTTTGATCAACGAC
CTTTTGGAAACTTCGGCTTCCCCTGGAGAGAGCGAGATTCTCCGCGCTGTAGAAGTCACCATTG
TTGTGCACGACGACATCATTCCGTGGCGTTATCCAGCTAAGCGCGAACTGCAATTTGGAGAATG
GCAGCGCAATGACATTCTTGCAGGTATCTTCGAGCCAGCCACGATCGACATTGATCTGGCTATC
TTGCTGACAAAAGCAAGAGAACATAGCGTTGCCTTGGTAGGTCCAGCGGCGGAGGAACTCTTTG
ATCCGGTTCCTGAACAGGATCTATTTGAGGCGCTAAATGAAACCTTAACGCTATGGAACTCGCC
GCCCGACTGGGCTGGCGATGAGCGAAATGTAGTGCTTACGTTGTCCCGCATTTGGTACAGCGCA
GTAACCGGCAAAATCGCGCCGAAGGATGTCGCTGCCGACTGGGCAATGGAGCGCCTGCCGGCCC
AGTATCAGCCCGTCATACTTGAAGCTAGACAGGCTTATCTTGGACAAGAAGAAGATCGCTTGGC
CTCGCGCGCAGATCAGTTGGAAGAATTTGTCCATTACGTAAAAGGCGAGATCACCAAGGTAGTC
GGCAAATAACTGTCAGACCAAGTTTACTCATATATACTTTAGATTGATTTAAAACTTCATTTTT
AATTTAAAAGGATCTAGGTGAAGATCCTTTTTGATAATCTCATGACCAAAATCCCTTAACGTGA
GTTTTCGTTCCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGGGGGTTCGTG
CACACAGCCCAGCTTGGAGCGAACGACCTACACCGAACTGAGATACCTACAGCGTGAGCTATGA
GAAAGCGCCACGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCCTGTCGGGTT
TCGCCACCTCTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCACATGTTCT
TTCCTGCGTTATCCCCTGATTCTGTGGATAACCGTATTACCGCCTTTGAGTGAGCTGATACCGC
TCGCCGCAGCCGAACGACCGAGCGCAGCGAGTCAGTGAGCGAGGAAGCGGAAGAGCGCCCAATA
CGCAAACCGCCTCTCCCCGCGCGTTGGCCGATTCATTAATCACTCTGTGGTCTCAGCTTGCTGT
AAAGAAATTGATGGGCAGTGGGCTTTTGTTACTAGTTAGTAGGAGAGGTTGCTTCAGTTTCGTC
CGTACCTGTTCTTGACCTTCTGTTTCTGGAGTCTGTACTCCGTTTGTTGTAAAGTCTTGTCCTT
TTTTTAAAACTTCTTTCTATCCACTGTTGAATGAGCCAGTAGATGCTGTCCTGTTACGCGTTTC
TCTTCTCTTGCACATGCACAGTCTCCGTTTTGTAGGATGCTGAACGAAGCTCTCGGGTTTATGG
AGGTCAATCCCTAAGTATTGTCGATTCAAAAGGGTGATGTTTTTTTCCCCCAACAAAGCTCTTC
AGTGAGTTCAACCAAGTGGGTGAGATGTGTATAGGTTACTGGACAATCTTGTTGGTTTGGAGAG
GAGAAAAAGTAGCTATATTGATCTGTGCCAGTGCTAGCACAGGGAGAGTCTTATCTTTTTGGGT
TAGTGTTACAGCTAGATGATTGAGATGATCATCTGCACTTGATTTGATCAGCTGGTTTTGTCTT
TGTAAGATTAGCCTGTCACTTGACGAAAAAAAGCGGTTTGTCTGTCCTCGGTTACGATTCAGAC
TGGTTTGGATGACGTCCATATTAAGATCCTGTATTTACGTTTGCTGCTCTCATTTTCTGCAAGC
TTTCCGAGGATGTCCAAAAGCTCGCTTGAGACCACGAA
Exemplary Epipremnum aureum agropine Histone H3 terminator
(Ter7.3)
SEQ ID NO: 410
GCTGTAAAGAAATTGATGGGCAGTGGGCTTTTGTTACTAGTTAGTAGGAGAGGTTGCTTCAGTT
TCGTCCGTACCTGTTCTTGACCTTCTGTTTCTGGAGTCTGTACTCCGTTTGTTGTAAAGTCTTG
TCCTTTTTTTAAAACTTCTTTCTATCCACTGTTGAATGAGCCAGTAGATGCTGTCCTGTTACGC
GTTTCTCTTCTCTTGCACATGCACAGTCTCCGTTTTGTAGGATGCTGAACGAAGCTCTCGGGTT
TATGGAGGTCAATCCCTAAGTATTGTCGATTCAAAAGGGTGATGTTTTTTTCCCCCAACAAAGC
TCTTCAGTGAGTTCAACCAAGTGGGTGAGATGTGTATAGGTTACTGGACAATCTTGTTGGTTTG
GAGAGGAGAAAAAGTAGCTATATTGATCTGTGCCAGTGCTAGCACAGGGAGAGTCTTATCTTTT
TGGGTTAGTGTTACAGCTAGATGATTGAGATGATCATCTGCACTTGATTTGATCAGCTGGTTTT
GTCTTTGTAAGATTAGCCTGTCACTTGACGAAAAAAAGCGGTTTGTCTGTCCTCGGTTACGATT
CAGACTGGTTTGGATGACGTCCATATTAAGATCCTGTATTTACGTTTGCTGCTCTCATTTTCTG
CAAGCTTTCCGAGGATGTCCAAAAGCTGCATTTTTTTTTTGTCGTTGGTAAATGTTACTTTCGA
TAATTTTAAGGTTGTGGCTGAGTGATACGAGGTGTTTTCTCGAAGATAATGGTCTTAGAGTTTT
ATTCTTGGCCTTCCACAAAAGGCAAAAAAAAGCTAACTCAAATGAGTTCTTAGTGTTGAGGTC

### Enhancers

In some instances, a vector can include an enhancer sequence. The term “enhancer” refers to a nucleotide sequence that can increase the level of transcription of a nucleic acid encoding a protein of interest. Enhancer sequences (generally 50-1500 bp in length) generally increase the level of transcription by providing additional binding sites for transcription-associated proteins (e.g., transcription factors). Unlike promoter sequences, in some embodiments certain enhancer sequences can act at much larger distance away from the transcription start site (e.g., as compared to a promoter). In some embodiments, an enhancer sequence is found within an intronic sequence. In some embodiments, an enhancer is an intronic sequence. In some embodiments, enhancers may act to decrease transcript degradation and/or silencing. In some embodiments, an enhancer may be inserted into the 5′ UTR of a vector. In some embodiments, an enhancer may be incorporated into a coding region of a transgene. In some embodiments, an intron acting as an enhancer may be an intron from a DEM1 gene, a DEM2 gene, a TCH3 gene, and/or a TRP1 gene. In some embodiments, additional non-limiting examples of enhancers include a RSV enhancer, a CMV enhancer, and/or a SV40 enhancer.

In some embodiments, an enhancer sequence is listed herein as set forth in SEQ ID NO: 56. In some embodiments, an enhancer sequence is at least 85%, 90%, 95%, 98% or 99% identical to an enhancer sequence represented by SEQ ID NO: 56. In some embodiments, an enhancer sequence is a characteristic portion of SEQ ID NO: 56.

Exemplary enhancer sequence, an Arabidopsis
thaliana DEMI intronic nucleotide sequence.
SEQ ID NO: 56
GTAAGCAGAACTCTAGTTGCAGTGTATATTCTTGCTGAGAAAGTGACATT
CTTGAAATTTTCATGTTTTGCTCATAGCATAAGTGCATATAATATTGAAG
TCTTAAGAATTTTTGTGGAAATTGAATTATAGTGTTCCTCAGTTGCCTTG
TGTTTCAACCTTGATTTTTGATAGAGGAACTTTTACTACTGTTGAATCAT
TCATCAATTGAAATAACTTTTTACTAATAGTTGATTCCTGACTCTTTTTG
TCTATCTTTTCTTGTTGAAAATGTCGATATATAG

### Flanking Untranslated Regions, 5′ UTRs and 3′ UTRs

In some embodiments, any of the vectors described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the vectors, compositions, kits, or methods as described herein to enhance or otherwise modulate the expression of a protein.

Natural 5′ UTRs include a sequence that plays a role in translation initiation. In some embodiments, a 5′ UTR can comprise sequences, like Kozak sequences, which are commonly known to be involved in the process by which the ribosome initiates translation of many genes. Kozak sequences have the consensus sequence CCR (A/G) CCAUGG, where R is a purine (A or G) three bases upstream of the start codon (AUG), and the start codon is followed by another “G”. In some embodiments, 5′ UTRs have also been known to form secondary structures that are involved in elongation factor binding.

In some embodiments, 5′ UTR is one listed herein as set forth in SEQ ID NOs: 57-60. In some embodiments, a 5′ UTR sequence is at least 85%, 90%, 95%, 98% or 99% identical to a 5′ UTR sequence represented by any one of SEQ ID NOs: 57-60. In some embodiments, a 5′ UTR sequence is a characteristic portion of any one of SEQ ID NOs: 57-60.

Exemplary Tobacco Mosaic Virus (TMV) 5′-leader
sequence (Omega).
SEQ ID NO: 57
GTATTTTTACAACAATTACCAACAACAACAAACAACAAACAACATTACAA
TTACTATTTACAATTAC
Exemplary Arabidopsis thaliana Alcohol
Dehydrogenase 5′ UTR.
SEQ ID NO: 58
TACATCACAATCACACAAAACTAACAAAAGATCAAAAGCAAGTTCTTCAC
TGTTGATA
Exemplary Nicotiana tabacum Alcohol Dehydrogenase
5′ UTR.
SEQ ID NO: 59
GTCTATTTCTCAGTATTCAGAAACAACAAAAGTTCTTCTCTACATAAAAT
TTTCCTATTTTAGTGATCAGTGAAGGAAATCAAGAAAAATAA
Exemplary Oryza sativa Alcohol Dehydrogense 5′
UTR.
SEQ ID NO: 60
GAATTCCAAGCAACGAACTGCGAGTGATTCAAGAAAAAAGAAAACCTGAG
CTTTCGATCTCTACGGAGTGGTTTCTTGTTCTTTGAAAAAGAGGGGGATT
A

### Internal Ribosome Entry Sites (IRES), Secretion Signals, and Cleavage Signals

In some embodiments, a vector encoding a protein can include an internal ribosome entry site (IRES). An IRES forms a complex secondary structure that allows translation initiation to occur from any position with an mRNA immediately downstream from where the IRES is located (see, e.g., Pelletier and Sonenberg, Mal. Cell. Biol. 8 (3): 1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15 (13): 1593-612, 2001, each of which is incorporated in its entirety herein by reference.

In some embodiments, a vector provided herein can include secretion signals, cleavage sites, and/or linker sequences. In some embodiments, these sites are functional in a translated protein, and result in post-translational modifications and/or processing events. In some embodiments, constructs as described herein are translated into a relatively long precursor polypeptide, such a precursor polypeptide may then undergo post translational modifications and/or processing, which may involve endogenous cellular enzymatic actions. Such a processing step may produce multiple peptides, the biological function of such peptides may be accomplished either solely by one peptide, or by the function of multiple peptides acting in concert.

In some embodiments, vectors provided herein include a signal peptide. In some embodiments, a signal peptide may be a signal sequence, targeting signal, localization signal, localization sequence, transit peptide, leader sequence or leader peptide. In some embodiments, such a sequence is generally short (e.g., approximately 15-60 amino acids in length). In some embodiments, such a signal peptide is present at the N-terminus of a peptide of interest. In some embodiments, more than one signal peptide may exist in a translational product. In some embodiments, an exemplary signal peptide comprises a localization signal. In some embodiments, such an amino acid sequence is represented by any one of SEQ ID NOs: 61-63, and can be 95%, 90%, 85%, 80%, or 75% identical to such a sequence. One skilled in the art will recognize that alternative localization signal sequences exist, and may be incorporated into vectors as described herein.

Exemplary Chloroplast localization signal amino
acid sequence
SEQ ID NO: 61
ASSMLSSAAVVTSPAQATMVAPFTGLKSSASFPVTRKANNDITSITSNGG
RVSC
Exemplary Mitochondria localization signal amino
acid sequence
SEQ ID NO: 62
MAMAVFRREGRRLLPSIAARPIAAIRSPLSSDQEEGLLGVRSISTQVVRN
R
Exemplary Peroxisome localization signal amino
acid sequence
SEQ ID NO: 63
MEKAIERQRVLLEHLRPSSSSSHNYEASLSASACLAGDSAAYQRTSLYG

In some embodiments, vectors provided herein include a linker peptide. In some embodiments, a linker peptide is utilized to join two or more functional peptides in a translational product. In some embodiments, such a linker peptide may include additional functional sequences, such as recognition sequences for endogenous peptidases. In some embodiments, a linker peptide may fuse two polypeptides together indefinitely. In some embodiments, a linker peptide sequence may be one amino acid in length, two amino acids in length, three amino acids in length, four amino acids in length, five amino acids in length, six amino acids in length, seven amino acids in length, eight amino acids in length, nine amino acids in length, ten amino acids in length, eleven amino acids in length, twelve amino acids in length, thirteen amino acids in length, fourteen amino acids in length, fifteen amino acids in length, sixteen amino acids in length, seventeen amino acids in length, eighteen amino acids in length, nineteen amino acids in length, or twenty amino acids in length. In some embodiments, a linker peptide sequence may be up to fifty amino acids in length. One skilled in the art will recognize that alternative linker sequences exist (functional or not), and may be incorporated into vectors as described herein.

In some embodiments, vectors provided herein include a peptide sequence that induces polypeptide cleavage and/or failure to form a peptide linkage during translation. In some embodiments, vectors as described herein may include a self-cleaving peptide, that in some embodiments may be a 2A self-cleaving peptide. In some embodiments, such a peptide is approximately 18 to 22 amino acids in length, e.g., 18 amino acids in length, 19 amino acids in length, 20 amino acids in length, 21 amino acids in length, or 22 amino acids in length. In some embodiments, such a peptide may induce ribosomal skipping during translation of a protein. In some embodiments, a 2A self-cleaving peptide is represented by a core sequence motif of DxExNPGP, and are found endogenously in a range of viral families. In some embodiments, a self-cleaving peptide generates polyproteins from a single transcript by causing the ribosome to fail at making a peptide bond. In some embodiments, a self-cleaving and/or cleavage signal is represented by any one of SEQ ID NOs: 64-69, or a sequence sharing approximately 95%, 90%, 80%, 75%, 70%, 65%, 60%, 55%, or 50% identity. One skilled in the art will recognize that alternative peptide cleavage sequences exist (self-cleaving or requiring the aid of endogenous cellular machinery), and may be incorporated into vectors as described herein.

-Exemplary Cleavage signal nucleotide sequence
SEQ ID NO: 64
GGCTCTGGCGAAGGCAGAGGCAGCCTGCTTACATGTGGCGACGTGGAAG
AGAACCCCGGACCT
-Exemplary Cleavage signal amino acid sequence
SEQ ID NO: 65
GSGEGRGSLLTCGDVEENPGP
-Exemplary Cleavage signal nucleotide sequence
SEQ ID NO: 66
GCCCCGGTGAAGCAGACCCTGAACTTCGACCTGCTGAAGCTGGCGGGCG
ACGTGGAGAGCAACCCGGGCCCC
-Exemplary Cleavage signal amino acid sequence
SEQ ID NO: 67
APVKQTLNFDLLKLAGDVESNPGP

In some embodiments, a ‘remnant’ 2A residue appended to the carboxyl terminus of the processed proteins can be removed by fusing an engineered mini-intein with the 2A sequence through a linker to create an ‘IntF2A’ self-excising domain. In some embodiments, an IntF2A enables co-translational cleavage via 2A's translational recoding activity, followed by post-translational autocatalytic cleavage via intein at its N-terminal junction (Zhang et al., Plant Biotechnology, 2017; incorporated herein by reference in its entirety).

-Exemplary IntF2A nucleotide sequence
SEQ ID NO: 68
TGTCTATCCTTTGGAACAGAGATATTGACAGTGGAATATGGCCCGTTACCAATAGGCAAAATCG
TGTCAGAAGAGATCAATTGCTCAGTCTATTCTGTTGATCCTGAGGGTAGAGTTTATACACAAGC
CATTGCGCAATGGCATGATAGAGGCGAACAAGAAGTCTTGGAATATGAATTAGAGGACGGGAGC
GTCATTAGGGCAACAAGTGATCATAGGTTTCTTACTACAGATTATCAACTTCTCGCCATTGAGG
AAATTTTTGCCCGACAGCTAGATCTCCTGACACTCGAAAATATTAAACAAACCGAGGAAGCGTT
GGATAATCATCGCCTCCCGTTTCCTCTCCTAGATGCAGGGACAATTAAGATGGTTAAAGTGATT
GGGAGGAGATCACTTGGTGTGCAAAGGATTTTTGATATAGGGCTCCCTCAGGACCACAACTTCT
TACTGGCTAACGGGGCAATCGCGGCAGCTTGTTCATGTGGTAGTGGGTCACGGGTAACTGAGTT
ACTTTATAGGATGAAGCGAGCTGAAACCTATTGCCCAAGACCCCTTTTGGCGATTCATCCTACA
GAAGCACGCCACAAACAAAAAATTGTGGCCCCAGTTAAACAACTTCTCAATTTTGACCTTTTGA
AGTTGGCCGGTGACGTCGAATCTAACCCCGGCCCT
-Exemplary IntF2A amino acid sequence
SEQ ID NO: 69
CLSFGTEILTVEYGPLPIGKIVSEEINCSVYSVDPEGRVYTQAIAQWHDRGEQEVLEYELEDGS
VIRATSDHRFLTTDYQLLAIEEIFARQLDLLTLENIKQTEEALDNHRLPFPLLDAGTIKMVKVI
GRRSLGVORIFDIGLPQDHNFLLANGAIAAACSCGSGSRVTELLYRMKRAETYCPRPLLAIHPT
EARHKQKIVAPVKQLLNFDLLKLAGDVESNPGP

### Splice Sites and Introns

In some embodiments, a vector provided herein can include splice donor and/or splice acceptor sequences. In some embodiments, such a splice donor and/or splice acceptor sequence may be functional during RNA processing occurring during and/or following transcription. In some embodiments, splice sites are involved in trans-splicing. In some embodiments, splices sites are involved in cis-splicing.

### Additional Sequences

In some embodiments, vectors of the present disclosure may include one or more cloning sites. In some such embodiments, cloning sites may not be fully removed prior to administration to a subject (e.g., a cell). In some embodiments, cloning sites may have functional roles, e.g., including as linker sequences, cleavage sequence, or as portions of a Kozak site. As will be appreciated by those skilled in the art, cloning sites may vary significantly in primary sequence while retaining their desired function. In some embodiments, vectors may contain any appropriate combination of cloning sites.

### Reporter Sequences or Elements

In some embodiments, vectors provided herein can optionally include a sequence encoding a reporter gene that may encode polypeptides and/or proteins (“a reporter sequence”). In some embodiments, reporter genes impart a distinct phenotype to cells expressing the reporter and thus allow transformed cells to be distinguished from cells that do not have the reporter. Such genes may encode, for example, a selectable and/or screenable reporter. In some embodiments, nucleic acid vectors comprise a reporter that allows selecting and/or screening of transformed cells.

In some embodiments, a transformed cell is grown in culture medium under conditions that select for cells that either have (positive selection) or do not have (negative selection) the reporter. In some embodiments, a combination of positive and negative selection is used. In some so-called positive selection schemes, most cells in a population are unable reproduce, e.g., because they lack the ability to use a nutrient (such as, for example, a carbon source) present in the selection medium. In some of these schemes, the selectable reporter confers an ability to use a limiting nutrient. Thus, in some embodiments, cells that have the selectable reporter gain an advantage over other cells in the population and therefore can be selected for. In some so-called negative screening/selection schemes, most cells in a population are unable to divide because of the effects of a toxic agent (such as, for example, an antibiotic present in the selection medium). In these schemes, the selectable reporter confers an ability to overcome the toxicity (for example, by blocking uptake or by chemically modifying the toxic agent). Thus, in some embodiments, cells that have the selectable reporter gain an advantage over other cells in the population and therefore can be selected for. In some embodiments, a transformed cell undergoing selection is a prokaryotic cell, e.g., such as E. coli or an Agrobacterium etc. In some embodiments, a transformed cell undergoing selection is a eukaryotic cell, such as a plant cell, yeast (for example, S. cerevisiae), mammalian cell, or insect cell. In some embodiments, a characteristic phenotype allows the identification of cells of interest, groups of cells, tissues, organs, plant parts or whole plants containing a vector of interest.

In some embodiments, vectors may include one or more nucleotide sequences encoding an appropriate selection and/or screening marker. In some embodiments, an appropriate selection marker may be encoded by nptII and/or kana and provide resistance to kanamycin. In some embodiments, an appropriate selection marker may be encoded by hpt and provide resistance to hyromycin. In some embodiments, an appropriate selection marker may be encoded by bar and provide resistance to phosphinothricin. In some embodiments, an appropriate selection marker may be encoded by gox and provide resistance to glyphosate. In some embodiments, an appropriate selection marker system includes neomycin phosphotransferase. In some embodiments, an appropriate selection marker system includes hygromycin phosphotransferase. In some embodiments, an appropriate selection marker system includes phosphoinothricin acetyltransferase. In some embodiments, an appropriate selection marker system includes glyphosate oxidoreductase.

Many examples of suitable reporter genes are known in the art and can be used in screening and/or selection schemes during methods described herein and/or during creation of compositions described herein. Reagents such as appropriate components of selection media are also known in the art. Examples of such reporter genes include, but are not limited to, phosphomannose isomerase, phosphinothricin, neomycin phosphotransferase, hygromycin phosphotransferase, enolpyruvoyl-shikimate-3-phosphate synthetase, etc.

For example, phosphomannose isomerase (PMI) catalyses the interconversion of mannose 6-phosphate and fructose 6-phosphate in prokaryotic and eukaryotic cells. After uptake, mannose is phosphorylated by endogenous hexokinases to mannose-6-phosphate. Accumulation of mannose-6-phosphate leads to a block in glycolysis by inhibition of phosphoglucose-isomerase, resulting in severe growth inhibition. Phosphomannose-isomerase is encoded by the manA gene from Escherichia coli and catalyzes the conversion of mannose-6-phosphate to fructose-6-phosphate, an intermediate of glycolysis. On media containing mannose, manA expression in transformed plant cells relieves the growth inhibiting effect of mannose-6-phosphate accumulation and permits utilization of mannose as a source of carbon and energy, allowing transformed cells to grow.

In some embodiments, reporter genes encode proteins that generate a detectable phenotype. Non-limiting examples of suitable reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. Alternatively or additionally, a reporter gene can provide some other visibly reactive response (e.g., may cause a distinctive appearance such as color or growth pattern relative to organisms or cells not expressing the selectable reporter gene in the presence of some substance, either as applied directly to the organism or cells or as present in the tissue or cell growth media). For example, it is known in the art that transcriptional activators of anthocyanin biosynthesis, operably linked to a suitable promoter in a vector, have widespread utility as non-phytotoxic markers for plant cell transformation.

In some embodiments, a reporter gene is an enhanced green fluorescence protein (eGFP) according to SEQ ID NO: 71, potentially encoded by SEQ ID NO: 70 or a codon optimized version thereof. In some embodiments, a reporter gene is an mCherry protein according to SEQ ID NO: 73, potentially encoded by SEQ ID NO: 72 or a codon optimized version thereof. In some embodiments, a reporter gene is an mRuby2 protein according to SEQ ID NO: 75, potentially encoded by SEQ ID NO: 74 or a codon optimized version thereof. In some embodiments, a reporter gene is an RRvT protein according to SEQ ID NO: 77, potentially encoded by SEQ ID NO: 76 or a codon optimized version thereof. In some embodiments, a reporter gene is an mTFP1 protein according to SEQ ID NO: 79, potentially encoded by SEQ ID NO: 80 or a codon optimized version thereof.

In some embodiments, a reporter gene may be but is not limited to eGFP, mCherry, mRubyd2, RRvT, mTFP1, RFP611, dTFP0.2, meffCFP, folding reporter GFP, ccalOFP1, tdKatushka2, vsfGFP-0, eYGFPuv, or any combination thereof.

In some embodiments, when reporter genes are associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, a reporter sequence is the LacZ gene, and the presence of a vector carrying the LacZ gene in a plant cell is detected by assays for beta-galactosidase activity. When the reporter is a fluorescent protein (e.g., green fluorescent protein) or luciferase, the presence of a vector carrying the fluorescent protein or luciferase in a plant cell may be measured by fluorescent techniques (e.g., fluorescent microscopy or FACS) or light production in a luminometer (e.g., a spectrophotometer or an IVIS imaging instrument). In some embodiments, a reporter sequence can be used to verify the tissue-specific targeting capabilities and tissue-specific promoter regulatory and/or control activity of any of the vectors described herein.

In some embodiments, a reporter sequence is a FLAG tag (e.g., a 3×FLAG tag), and the presence of a vector carrying the FLAG tag in a plant cell is detected by protein binding or detection assays (e.g., Western blots, immunohistochemistry, radioimmunoassay (RIA), mass spectrometry).

-Exemplary eGFP reporter nucleotide sequence
SEQ ID NO: 70
ATGGTGAGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCA
AGTCCGCCATGCCCGAAGGCTACGTCCAGGAGCGCACCATCTTCTTCAAGGACGACGGCAACTA
CAAGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCCGCCACAA
CATCGAGGACGGCAGCGTGCAGCTCGCCGACCACTACCAGCAGAACACCCCCATCGGCGACGGC
CCCGTGCTGCTGCCCGACAACCACTACCTGAGCACCCAGTCCGCCCTGAGCAAAGACCCCAACG
AGAAGCGCGATCACATGGTCCTGCTGGAGTTCGTGACCGCCGCCGGGATCACTCTCGGCATGGA
CGAGCTGTACAAG
-Exemplary eGFP reporter amino acid sequence
SEQ ID NO: 71
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKG
IDFKEDGNILGHKLEYNYNSHNVYIMADKQKNGIKVNFKIRHNIEDGSVQLADHYQQNTPIGDG
PVLLPDNHYLSTQSALSKDPNEKRDHMVLLEFVTAAGITLGMDELYK
-Exemplary mCherry reporter nucleotide sequence
SEQ ID NO: 72
ATGGTGAGCAAGGGCGAGGAGGATAACATGGCCATCATCAAGGAGTTCATGCGCTTCAAGGTGC
ACATGGAGGGCTCCGTGAACGGCCACGAGTTCGAGATCGAGGGCGAGGGCGAGGGCCGCCCCTA
CGAGGGCACCCAGACCGCCAAGCTGAAGGTGACCAAGGGTGGCCCCCTGCCCTTCGCCTGGGAC
ATCCTGTCCCCTCAGTTCATGTACGGCTCCAAGGCCTACGTGAAGCACCCCGCCGACATCCCCG
ACTACTTGAAGCTGTCCTTCCCCGAGGGCTTCAAGTGGGAGCGCGTGATGAACTTCGAGGACGG
CGGCGTGGTGACCGTGACCCAGGACTCCTCCCTGCAGGACGGCGAGTTCATCTACAAGGTGAAG
CTGCGCGGCACCAACTTCCCCTCCGACGGCCCCGTAATGCAGAAGAAAACCATGGGCTGGGAGG
CCTCCTCCGAGCGGATGTACCCCGAGGACGGCGCCCTGAAGGGCGAGATCAAGCAGAGGCTGAA
GCTGAAGGACGGCGGCCACTACGACGCTGAGGTCAAGACCACCTACAAGGCCAAGAAGCCCGTG
CAGCTGCCCGGCGCCTACAACGTCAACATCAAGTTGGACATCACCTCCCACAACGAGGACTACA
CCATCGTGGAACAGTACGAACGCGCCGAGGGCCGCCACTCCACCGGCGGCATGGACGAGCTGTA
CAAGTAA
-Exemplary mCherry reporter amino acid sequence
SEQ ID NO: 73
MVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVK
LRGTNFPSDGPVMQKKTMGWEASSERMYPEDGALKGEIKQRLKLKDGGHYDAEVKTTYKAKKPV
QLPGAYNVNIKLDITSHNEDYTIVEQYERAEGRHSTGGMDELYK
-Exemplary mRuby reporter nucleotide sequence
SEQ ID NO: 74
ATGGTGTCAAAAGGTGAGGAGCTAATCAAAGAGAACATGCGAATGAAAGTGGTCATGGAAGGGA
GCGTAAACGGCCACCAGTTCAAATGCACAGGCGAGGGCGAGGGCAACCCATACATGGGTACGCA
GACCATGAGGATAAAAGTAATCGAGGGTGGTCCGTTGCCATTCGCCTTCGACATCCTGGCAACC
TCGTTCATGTACGGGAGTCGAACATTCATCAAATACCCAAAAGGTATACCGGACTTCTTCAAAC
AGAGTTTCCCGGAAGGTTTCACCTGGGAGCGGGTCACAAGGTACGAGGACGGTGGTGTCGTGAC
AGTAATGCAGGACACATCCTTAGAGGACGGTTGCCTGGTCTACCACGTCCAGGTGCGTGGCGTC
AACTTCCCCTCAAACGGCCCAGTAATGCAGAAGAAAACCAAAGGTTGGGAGCCGAACACAGAGA
TGATGTACCCGGCGGACGGTGGCCTGCGTGGTTACACACACATGGCATTAAAAGTGGACGGTGG
TGGTCACCTCTCGTGCTCGTTCGTCACAACCTACCGAAGCAAGAAAACGGTCGGGAACATCAAA
ATGCCGGGTATACACGCAGTCGACCACCGTCTCGAGCGTTTAGAGGAGAGCGACAACGAGATGT
TCGTCGTGCAGCGAGAGCACGCAGTGGCCAAATTCGCGGGTCTAGGCGGCGGGATGGACGAGTT
ATACAAATGA
-Exemplary mRuby reporter amino acid sequence
SEQ ID NO: 75
MVSKGEELIKENMRMKVVMEGSVNGHQFKCTGEGEGNPYMGTQTMRIKVIEGGPLPFAFDILAT
SFMYGSRTFIKYPKGIPDFFKQSFPEGFTWERVTRYEDGGVVTVMQDTSLEDGCLVYHVQVRGV
NFPSNGPVMQKKTKGWEPNTEMMYPADGGLRGYTHMALKVDGGGHLSCSFVTTYRSKKTVGNIK
MPGIHAVDHRLERLEESDNEMFVVQREHAVAKFAGLGGGMDELYK
-Exemplary RRvT reporter nucleotide sequence
SEQ ID NO: 76
ATGGTATCAAAAGGGGAAGAGGTGATCAAAGAGTTCATGCGTTTCAAAGTACGAATGGAAGGTT
CCATGAACGGGCACGAGTTCGAGATAGAGGGTGAGGGTGAGGGTAGGCCATACGAGGGCACACA
GACGGCCAAACTGAAAGTAACCAAAGGTGGCCCACTCCCATTCGCGTGGGACATCTTGAGTCCA
CAGTTCATGTACGGTAGCAAAGCCTACGTCAAACACCCGGCCGACATACCAGACTACAAGAAAC
TAAGTTTCCCAGAGGGGTTCAAATGGGAGCGAGTAATGAACTTCGAGGACGGCGGCCTGGTCAC
GGTGACCCAGGACTCGAGTTTACAGGACGGTACCTTGATATACAACGTCAAAATGCGGGGTACA
AACTTTCCCCCAGACGGCCCCGTAATGCAGAAGAAAACAATGGGTTGGGAAGCAAGCACAGAGC
GTTTGTACCCAAGGGACGGTGTGCTAAAAGGTGAGATCCACCAGGCACTAAAATTAAAAGACGG
CGGTCACTACCTAGTCGAGTTCAAAACCATATACATGGCGAAGAAACCCGTGCAGCTCCCAGGT
TACTACTACGTAGACACCAAATTAGACATCACGTCGCACAACGAGGACTACACGATCGTCGAGC
AGTACGAGCGTAGCGAGGGTCGACACCACCTCTTCCTATACGGTATGGACGAGCTCTACAAA
-Exemplary RRvT reporter amino acid sequence
SEQ ID NO: 77
MVSKGEEVIKEFMRFKVRMEGSMNGHEFEIEGEGEGRPYEGTQTAKLKVTKGGPLPFAWDILSP
QFMYGSKAYVKHPADIPDYKKLSFPEGFKWERVMNFEDGGLVTVTQDSSLQDGTLIYNVKMRGT
NFPPDGPVMQKKTMGWEASTERLYPRDGVLKGEIHQALKLKDGGHYLVEFKTIYMAKKPVQLPG
YYYVDTKLDITSHNEDYTIVEQYERSEGRHHLFLYGMDELYK
-Exemplary mTFP1 reporter nucleotide sequence
SEQ ID NO: 78
ATGGTCAGTAAAGGTGAGGAGACGACGATGGGTGTCATAAAACCAGACATGAAAATAAAACTGA
AAATGGAAGGTAACGTCAACGGCCACGCATTCGTAATCGAGGGTGAGGGTGAGGGGAAACCATA
CGACGGGACGAACACCATAAACCTGGAAGTGAAAGAGGGTGCCCCACTACCATTCTCATACGAC
ATCCTGACAACCGCGTTCGCCTACGGTAACAGGGCATTCACCAAATACCCCGACGACATCCCAA
ACTACTTCAAACAGTCATTCCCAGAGGGTTACAGTTGGGAGAGGACAATGACATTCGAGGACAA
AGGGATCGTGAAAGTGAAAAGCGACATCAGCATGGAAGAGGACTCCTTCATCTACGAGATCCAC
TTGAAAGGTGAGAACTTCCCACCCAACGGTCCCGTAATGCAGAAGAAAACAACCGGTTGGGACG
CATCAACCGAGCGGATGTACGTAAGGGACGGCGTCTTAAAAGGTGACGTGAAACACAAACTGCT
GTTGGAAGGTGGTGGGCACCACAGGGTCGACTTCAAAACCATATACCGAGCAAAGAAAGCCGTG
AAATTGCCAGACTACCACTTCGTCGACCACCGGATAGAGATACTAAACCACGACAAAGACTACA
ACAAAGTAACCGTGTACGAGAGTGCCGTAGCGCGAAACTCCACAGACGGCATGGACGAGCTGTA
CAAATGA
-Exemplary mTFP1 reporter amino acid sequence
SEQ ID NO: 79
MVSKGEETTMGVIKPDMKIKLKMEGNVNGHAFVIEGEGEGKPYDGTNTINLEVKEGAPLPFSYD
ILTTAFAYGNRAFTKYPDDIPNYFKQSFPEGYSWERTMTFEDKGIVKVKSDISMEEDSFIYEIH
LKGENFPPNGPVMQKKTTGWDASTERMYVRDGVLKGDVKHKLLLEGGGHHRVDFKTIYRAKKAV
KLPDYHFVDHRIEILNHDKDYNKVTVYESAVARNSTDGMDELYK
-Exemplary RFP611 reporter nucleotide sequence
SEQ ID NO: 80
ATGAACTCATTAATCAAAGAGAACATGCGTATGATGGTGGTCATGGAAGGCTCGGTCAACGGTT
ACCAGTTCAAATGCACAGGTGAGGGTGACGGTAACCCATACATGGGTACCCAGACAATGCGTAT
CAAAGTGGTAGAGGGCGGTCCATTGCCCTTCGCGTTCGACGTACTGGCAACCAGTTTCATGTAC
GGTTCAAAGACGTTCATCAAACACACCAAAGGTATACCCGACTTCTTCAAACAGTCATTCCCAG
AGGGTTTCACATGGGAGCGGGTGACGAGGTACGAGGACGGTGGTGTCATCACCGTGATGCAGGA
CACATCGCTCGAGGACGGCTGCTTGGTGTACCACGCCAAAGTGACGGGCGTCAACTTCCCCAGT
AACGGTGCAGTCATGCAGAAGAAAACGAAAGGGTGGGAGCCAAACACGGAGATGTTATACCCCG
CCGACGGCGGTCTGCGAGGTTACAGTCAGATGGCCCTGAACGTGGACGGGGGGGGTTACTTGTC
GTGCTCCTTCGAGACAACGTACAGGAGTAAGAAAACGGTAGAGAACTTCAAAATGCCAGGCTTC
CACTTCGTCGACCACCGTTTGGAGCGTCTCGAGGAGAGTGACAAAGAGATGTTCGTGGTCCAGC
ACGAGCACGCCGTGGCAAAATTCTGCGATCTCCCATCAAAACTCGGTAGGCTGTAG
-Exemplary RFP611 reporter amino acid sequence
SEQ ID NO: 81
MNSLIKENMRMMVVMEGSVNGYQFKCTGEGDGNPYMGTQTMRIKVVEGGPLPFAFDVLATSFMY
GSKTFIKHTKGIPDFFKQSFPEGFTWERVTRYEDGGVITVMQDTSLEDGCLVYHAKVTGVNFPS
NGAVMQKKTKGWEPNTEMLYPADGGLRGYSQMALNVDGGGYLSCSFETTYRSKKTVENFKMPGF
HFVDHRLERLEESDKEMFVVQHEHAVAKFCDLPSKLGRL
-Exemplary dTFP0.2 reporter nucleotide sequence
SEQ ID NO: 82
ATGGTGTCGAAAGGTGAGGAGACGACTATGGGCGTGATCAAACCAGACATGAAAATCAAACTGA
AAATGGAAGGTAACGTCAACGGTCACGCATTCGTAATCGAGGGTGAAGGGGAAGGCAAACCATA
CGACGGTACAAACACAGTCAACTTGGAAGTCAAAGAGGGCGCACCACTGCCGTTCAGTTACGAC
ATCCTCAGTAACGCATTCCAGTACGGTAACCGTGCATTCACAAAATACCCCGACGACATCGCAA
ACTACTTCAAACAGTCATTCCCAGAGGGTTACAGCTGGGAGCGGACAATGACATTCGAGGACAA
AGGGATCGTAAAAGTGAAAAGTGACATATCAATGGAAGAGGACTCATTCATCTACGAGATAAGG
TTAAAAGGGAAGAACTTCCCACCAAACGGTCCAGTGATGCAGAAGAAAACACTCAAATGGGAGC
CATCAACCGAGATCCTCTACGTGCGTGACGGTGTCTTGGTGGGTGACATCTCACACAGTTTGCT
GCTCGAGGGTGGCGGTCACTACCGGTGCGACTTCAAAACCATCTACAAAGCCAAGAAAGTAGTC
AAACTGCCCGACTACCACTTCGTCGACCACAGGATAGAGATCTTGAACCACGACAAAGACTACA
ACAAAGTCACATTGTACGAGAACGCAGTGGCCCGATACAGCCTGTTACCACCACAGGCCGGGAT
GGACGAGTTGTACAAATGA
-Exemplary dTFP0.2 reporter amino acid sequence
SEQ ID NO: 83
MVSKGEETTMGVIKPDMKIKLKMEGNVNGHAFVIEGEGEGKPYDGTNTVNLEVKEGAPLPFSYD
ILSNAFQYGNRAFTKYPDDIANYFKQSFPEGYSWERTMTFEDKGIVKVKSDISMEEDSFIYEIR
LKGKNFPPNGPVMQKKTLKWEPSTEILYVRDGVLVGDISHSLLLEGGGHYRCDFKTIYKAKKVV
KLPDYHFVDHRIEILNHDKDYNKVTLYENAVARYSLLPPQAGMDELYK
-Exemplary meffCFP reporter nucleotide sequence
SEQ ID NO: 84
ATGGCATTGAGCAAACAGTCCCTACCCAGCGACATGAAATTGATCTACCACATGGACGGGAACG
TGAACGGTCACTCCTTCGTCATAAAAGGCGAGGGTGAGGGTAAACCATACGAGGGCACACACAC
AATAAAACTGCAGGTAGTCGAGGGTAGTCCGCTGCCGTTCAGCGCCGACATACTGTCAACCGTA
TTCCAGTACGGTAACCGATGCTTCACAAAATACCCACCAAACATAGTGGACTACTTCAAGAACT
CATGCTCCGGTGGTGGCTACAAATTCGGGCGTTCATTCCTATACGAGGACGGCGCGGTCTGCAC
AGCAAGTGGTGACATAACACTCAGTGCAGACAAGAAATCATTCGAGCACAAATCGAAATTCCTG
GGCGTGAACTTCCCAGCAGACGGCCCGGTGATGAAGAAAGAGACAACAAACTGGGAGCCATCAT
GCGAGAAAATGACGCCCAACGGCATGACGTTGATCGGGGACGTCACAGGCTTCTTATTAAAAGA
GGACGGGAAACGGTACAAATGCCAGTTCCACACCTTCCACGACGCCAAAGACAAAAGCAAGAAG
ATGCCGATGCCAGACTTCCACTTCGTGCAGCACAAAATAGAGCGGAAAGACCTGCCAGGTTCAA
TGCAGACATGGCGACTGACAGAGCACGCAGCCGCGTGCAAAACGTGCTTCACCGAGTGA
-Exemplary meffCFP reporter amino acid sequence
SEQ ID NO: 85
MALSKQSLPSDMKLIYHMDGNVNGHSFVIKGEGEGKPYEGTHTIKLQVVEGSPLPFSADILSTV
FQYGNRCFTKYPPNIVDYFKNSCSGGGYKFGRSFLYEDGAVCTASGDITLSADKKSFEHKSKFL
GVNFPADGPVMKKETTNWEPSCEKMTPNGMTLIGDVTGFLLKEDGKRYKCQFHTFHDAKDKSKK
MPMPDFHFVQHKIERKDLPGSMQTWRLTEHAAACKTCFTE
-Exemplary Folding Reporter GFP reporter nucleotide sequence
SEQ ID NO: 86
ATGAGTAAAGGTGAGGAACTGTTCACAGGCGTTGTACCGATCCTGGTGGAGTTAGACGGCGACG
TGAACGGTCACAAATTCTCAGTCAGTGGTGAGGGTGAGGGCGACGCCACATACGGTAAATTGAC
ACTGAAATTCATATGCACAACAGGTAAATTGCCCGTACCCTGGCCAACGTTGGTAACAACCCTA
ACGTACGGTGTCCAGTGCTTCTCGCGATACCCAGACCACATGAAACGTCACGACTTCTTCAAAA
GCGCGATGCCAGAGGGTTACGTCCAGGAGCGAACAATATCATTCAAAGACGACGGTAACTACAA
AACAAGGGCAGAGGTGAAATTCGAGGGTGACACATTAGTCAACCGAATAGAGTTAAAAGGTATC
GACTTCAAAGAGGACGGTAACATACTAGGTCACAAACTCGAGTACAACTACAACTCCCACAACG
TCTACATAACAGCGGACAAACAGAAGAACGGTATCAAAGCAAACTTCAAAATCAGGCACAACAT
CGAGGACGGCTCAGTGCAGCTCGCGGACCACTACCAGCAGAACACACCCATCGGTGACGGTCCG
GTCTTACTCCCCGACAACCACTACCTATCAACGCAGTCCGCCCTGAGTAAAGACCCAAACGAGA
AACGTGACCACATGGTCCTACTCGAGTTCGTAACAGCAGCGGGGATAACCCACGGTATGGACGA
GTTATACAAATGA
-Exemplary Folding Reporter GFP reporter amino acid sequence
SEQ ID NO: 87
MSKGEELFTGVVPILVELDGDVNGHKFSVSGEGEGDATYGKLTLKFICTTGKLPVPWPTLVTTL
TYGVQCFSRYPDHMKRHDFFKSAMPEGYVQERTISFKDDGNYKTRAEVKFEGDTLVNRIELKGI
DFKEDGNILGHKLEYNYNSHNVYITADKQKNGIKANFKIRHNIEDGSVQLADHYQQNTPIGDGP
VLLPDNHYLSTQSALSKDPNEKRDHMVLLEFVTAAGITHGMDELYK
-Exemplary ccalOFP1 reporter nucleotide sequence
SEQ ID NO: 88
ATGTCCCTCTCGAAACAAGTATTACCAAGAGACGTTAAAATGCGATTCCACATGGACGGTTGCG
TGAACGGCCACTCATTCACGATAGAAGGAGAGGGTACCGGGAAACCGTACGAGGGTAAGAAAAC
GTTGAAACTCAGGGTGACAAAAGGTGGTCCGCTACCGTTCGCCTTCGACATCCTGTCGGCGACC
TTCACGTACGGCAACAGGTGCTTCTGCGACTACCCAGAGGAGATGCCCGACTACTTCAAACAGA
GTTTACCAGAGGGTTACAGCTGGGAGAGGACGATGATGTACGAGGACGGTGCATGCTCAACAGC
GAGTGCCCACATCAGTTTGGACAAAGACTGCTTCATCCACAACAGTACATTCCACGGTGTGAAC
TTCCCAGCGAACGGCCCAGTCATGCAGAAGAAGGCGATGAACTGGGAGCCGAGCTCAGAGTTAA
TAACCCCATGCGACGGGATCTTGAAAGGCGACGTAACGATGTTCTTACTACAAGAGGGTGGTCA
CCGTCACAAATGCCAGTTCACAACTTCCTACAAAGCCCACAAAGCGGTCAAAATCCCGCCAAAC
CACATCATCGAGCACAGGTTGGTACGTAAAGAGGTGGGTGACGCAGTCCAGATCCAGGAGCACG
CAGTGGCGAAACACTTCACAGTCCAGATAAAAGAGGCGTGA
-Exemplary ccalOFP1 reporter amino acid sequence
SEQ ID NO: 89
MSLSKQVLPRDVKMRFHMDGCVNGHSFTIEGEGTGKPYEGKKTLKLRVTKGGPLPFAFDILSAT
FTYGNRCFCDYPEEMPDYFKQSLPEGYSWERTMMYEDGACSTASAHISLDKDCFIHNSTFHGVN
FPANGPVMQKKAMNWEPSSELITPCDGILKGDVTMFLLQEGGHRHKCQFTTSYKAHKAVKIPPN
HIIEHRLVRKEVGDAVQIQEHAVAKHFTVQIKEA
-Exemplary tdKatushka2 reporter nucleotide sequence
SEQ ID NO: 90
ATGTCAGAGTTGATAAAAGAGAACATGCACATGAAATTATACATGGAAGGTACCGTAAACAACC
ACCACTTCAAATGCACCTCAGAGGGAGAGGGTAAACCGTACGAGGGTACACAGACAATGAAAAT
CAAAGTGGTCGAGGGTGGTCCCCTACCATTCGCGTTCGACATCCTGGCCACCAGTTTCATGTAC
GGCTCAAAGACGTTCATAAACCACACACAGGGGATACCCGACTTCTTCAAACAGTCATTCCCAG
AGGGCTTCACCTGGGAGCGAATCACAACATACGAGGACGGCGGTGTGTTGACAGCAACGCAGGA
CACATCCCTGCAGAACGGTTGCATAATATACAACGTTAAAATAAACGGTGTCAACTTCCCATCG
AACGGGAGTGTGATGCAGAAGAAAACCTTAGGTTGGGAAGCCAACACCGAGATGTTGTACCCCG
CCGACGGCGGCCTACGGGGACACAGTCAGATGGCCTTAAAACTAGTGGGTGGTGGTTACCTACA
CTGCAGTTTCAAAACAACCTACCGTAGCAAGAAACCAGCGAAGAACCTCAAAATGCCAGGTTTC
CACTTCGTGGACCACCGTCTCGAGAGGATCAAAGAGGCGGACAAAGAGACATACGTGGAGCAGC
ACGAGATGGCGGTCGCGAAATACTGCGACCTACCATCCAAACTAGGTCACCGTTAG
-Exemplary tdKatushka2 reporter amino acid sequence
SEQ ID NO: 91
MSELIKENMHMKLYMEGTVNNHHFKCTSEGEGKPYEGTQTMKIKVVEGGPLPFAFDILATSFMY
GSKTFINHTQGIPDFFKQSFPEGFTWERITTYEDGGVLTATQDTSLQNGCIIYNVKINGVNFPS
NGSVMQKKTLGWEANTEMLYPADGGLRGHSQMALKLVGGGYLHCSFKTTYRSKKPAKNLKMPGF
HFVDHRLERIKEADKETYVEQHEMAVAKYCDLPSKLGHR
-Exemplary vsfGFP-0 reporter nucleotide sequence
SEQ ID NO: 92
ATGTCTAAAGGAGAGGAGTTGTTCACTGGTGTCGTGCCGATCCTGGTCGAGCTCGACGGTGACG
TCAACGGGCACAAATTCTCAGTCCGAGGTGAGGGCGAGGGTGACGCAACAAACGGTAAATTGAC
ACTGAAATTCATCTGCACGACGGGTAAATTACCGGTACCGTGGCCAACATTGGTGACGACACTG
ACATACGGTGTGCAGTGCTTCAGCCGATACCCCGACCACATGAAACGACACGACTTCTTCAAAT
CAGCAATGCCAGAGGGTTACGTACAGGAGAGGACGATCAGCTTCAAAGACGACGGCACCTACAA
AACCCGTGCGGAAGTGAAATTCGAGGGTGACACCTTGGTCAACCGAATCGAGTTGAAAGGTATC
GACTTCAAAGAGGACGGTAACATATTAGGTCACAAATTGGAGTACAACTTCAACAGTCACAACG
TCTACATCACAGCCGACAAACAGAAGAACGGTATCAAAGCCAACTTCAAAATCCGTCACAACGT
AGAGGACGGCTCCGTGCAGCTAGCGGACCACTACCAGCAGAACACGCCAATCGGGGACGGCCCC
GTACTGCTGCCAGACAACCACTACCTATCAACACAGAGCGTGCTCTCAAAAGACCCAAACGAGA
AACGGGACCACATGGTGTTGTTGGAGTTCGTAACGGCGGCAGGTATAGCGCAGGTGCAGTTGGT
AGAGTCAGGTGGGGCATTGGTACAGCCAGGTGGTTCACTGCGGTTATCATGCGCAGCATCAGGT
TTCCCGGTAAACAGGTACTCCATGCGATGGTACCGGCAGGCACCGGGTAAAGAGAGGGAGTGGG
TGGCGGGTATGTCCAGTGCGGGTGACAGGTCGTCGTACGAGGACTCAGTCAAAGGTAGGTTCAC
CATAAGTAGGGACGACGCACGAAACACCGTGTACCTGCAGATGAACAGTCTAAAACCAGAGGAC
ACAGCGGTGTACTACTGCAACGTCAACGTAGGTTTCGAGTACTGGGGTCAGGGTACGCAGGTGA
CAGTGTCGTGA
-Exemplary vsfGFP-O reporter amino acid sequence
SEQ ID NO: 93
MSKGEELFTGVVPILVELDGDVNGHKFSVRGEGEGDATNGKLTLKFICTTGKLPVPWPTLVTTL
TYGVQCFSRYPDHMKRHDFFKSAMPEGYVQERTISFKDDGTYKTRAEVKFEGDTLVNRIELKGI
DFKEDGNILGHKLEYNFNSHNVYITADKQKNGIKANFKIRHNVEDGSVQLADHYQQNTPIGDGP
VLLPDNHYLSTQSVLSKDPNEKRDHMVLLEFVTAAGIAQVQLVESGGALVQPGGSLRLSCAASG
FPVNRYSMRWYRQAPGKEREWVAGMSSAGDRSSYEDSVKGRFTISRDDARNTVYLQMNSLKPED
TAVYYCNVNVGFEYWGQGTQVTVS
-Exemplary eYGFPuv reporter nucleotide sequence
SEQ ID NO: 94
ATGACCACATTCAAAATCGAGAGTAGGATCCACGGTAACTTGAACGGCGAGAAATTCGAGCTAG
TAGGCGGTGGTGTAGGGGAAGAGGGAAGGCTCGAGATCGAGATGAAAACAAAAGACAAACCGTT
AGCATTCTCGCCATTCCTGTTGACAACGTGCATGGGTTACGGTTTCTACCACTTCGCTTCCTTC
CCGAAAGGTATAAAGAACATATACTTGCACGCAGCCACGAACGGCGGCTACACCAACACACGTA
AAGAGATATACGAGGACGGTGGTATACTGGAAGTCAACTTCAGGTACACGTACGAGTTCAACAA
AATCATCGGCGACGTGGAGTGCATAGGTCACGGCTTCCCCTCGCAGTCCCCAATCTTCAAAGAC
ACAATAGTCAAATCGTGCCCAACGGTGGACTTAATGCTGCCAATGAGCGGGAACATAATCGCCT
CATCCTACGCATACGCATTCCAGCTCAAAGACGGTAGTTTCTACACAGCCGAGGTCAAGAACAA
CATAGACTTCAAGAACCCAATACACGAGTCCTTCTCAAAATCCGGGCCGATGTTCACACACCGT
CGGGTTGAGGAGACACTAACAAAAGAGAACCTGGCAATAGTGGAGTACCAGCAGGTGTTCAACT
CGGCCCCGCGGGACATGTGA
-Exemplary eYGFPuv reporter amino acid sequence
SEQ ID NO: 95
MTTFKIESRIHGNLNGEKFELVGGGVGEEGRLEIEMKTKDKPLAFSPFLLTTCMGYGFYHFASF
PKGIKNIYLHAATNGGYTNTRKEIYEDGGILEVNFRYTYEFNKIIGDVECIGHGFPSQSPIFKD
TIVKSCPTVDLMLPMSGNIIASSYAYAFQLKDGSFYTAEVKNNIDFKNPIHESFSKSGPMFTHR
RVEETLTKENLAIVEYQQVFNSAPRDM

### Gene of Interest

In some embodiments, compositions and methods are provided herein comprise a gene of interest. In some embodiments, a gene of interest is nucleic acid coding sequence that codes for a protein of interest. In some embodiments, a protein of interest is a protein that may metabolize a pollutant (e.g., as described herein). In some embodiments, a protein of interest is a part of a metabolic pathway. In some embodiments, transgenic vectors as described herein comprise more than one protein of interest. In some embodiments, a transgenic vector comprises one gene of interest. In some embodiments, a transgenic vector comprises two genes of interest. In some embodiments, a transgenic vector comprises three genes of interest. In some embodiments, a transgenic vector comprises four genes of interest. In some embodiments, a transgenic vector comprises five genes of interest. In some embodiments, a transgenic vector comprises six genes of interest. In some embodiments, a transgenic vector comprises seven genes of interest. In some embodiments, a transgenic vector comprises eight genes of interest. In some embodiments a transgenic vector comprises nine genes of interest. In some embodiments, a transgenic vector comprises ten genes of interest. In some embodiments, more than one gene of interest are influence by the same regulatory elements. In some embodiments, each of more than one gene of interests in a transgenic vector is controlled by the same regulatory elements. In some embodiments, each of more than one gene of interests in a transgenic vector is controlled by unique regulatory elements.

In some embodiments a gene of interest may be, but is not limited to: ANT1, ANT1_mut, AtCaprice, atFDH-1.1, AtGlabra1, AtGlabra2, AtGlabra3, AtPAP1, AtStomagen, AtStomagen (Ea codon optimized), AtStomagen (Ea), AtWRI1, AtWRI4, Bar, Bmoa_AP, BMOA_PA, CaMYBA (Ea), CaMYC (Ea), ccalOFP1, CER1, CER6, CPH, CrtW, CrtW (Ea codon optimized), CrtW (Ea), CrtZ, CrtZ (Ea codon optimized), CrtZ (Ea), DAK_Cf, DAK_Ec, DAK_Pp, DAK2_Yeast, DAS_Canbo, Delila, Delila_mut, DHAK-2yeast, DHAK-cf, DHAK-ec, Dhak-PP, dTFP0.2, Dummy, EaFALDH, EaFALDH-IntF2A-AtFDH1.3 (Ea codon optimized), EaFALDH-IntF2a-AtFDH1.3 (Ea), EaZIP, EaZIP_mut, eYGFPuv, FALDH_10, FALDH_11, FALDH_9, FALDH_Ea*, FALDH-11, FALDH-9, FALDH-EA, FALDHP, FDH_3, FDH_3 (Chloro), FDH_3 (Cyto), FDH_Pp, FDH3, FDH3_cyto, FDH3_mito, FhMYB5 (Ea), FhTT8 L (Ea), Folding Reporter GFP, Formolase, GhPAP1, Glabra1, Glabra2, Glabra3, Glucoronidase, GUS, H3H, HispS, HPS/PHI_a, HPS/PHI_Bm (Ea), HPS/PHI_Bm fusion (Ea codon optimized), HPS/PHI_Mg fuqion (Ea codon optimized), HPS/PHIA, HPS-BM, HPS-MG, HPT (Ea codon optimized), KANA, Level M end-linker 2, Level M end-linker 3, Level M end-linker 4, Level M end-linker 5, Level M end-linker 7, Luz, mCherry, meffCFP, mRuby2, mTFP1, MYB306, Nanoluc, nptII (kana), NtMyb123, NtMyb23, OsGL1-1, OsX1, OsX2, P19, P35S-eGFP, P450_2E1, P450_RR, P450-2E1, P540_RR, PHE_OH, PHI-BM, PHI-MG, PPvUbi2-eGFP, PvUbi1+3-eGFP, PZmUbi1-eGFP, RFP611, Rosea_mut, Roseal, Roseal_mut, RRvT monomer, Tbual, TBUA1_Mp, tdKatushka2, tmoA_Pm, Tmoa_SP, TMOF_PM, To_Woolly, TOD_C₁, Tod-C₁, TodC1 (Ea codon optimized), TodC1 (Ea), toua_SP, TouA_SP_OX1, Toua-SP, TurboGFP, vsfGFP-0, VvMYBA5, VvMYBA6, ZmLc, ZmPl, SMH1, GLO1, GLO2, or any combination thereof.

### Gene of Interest Knockout or Knockdown

In some embodiments, compositions and methods are provided herein that utilize the silencing of endogenous plant transgene regulatory elements. In some embodiments, this may be performed using gene editing mechanisms such as TALENs, Zinc-Finger nucleases, and/or CRISPR mediated mutations (e.g., any mutation that creates a knock-down, knock-out, or otherwise reduced function allele).

In some embodiments, the gene RDR6 is targeted, this gene and its associated pathway have been implicated in the silencing of transgenes [Luo & Chen, Plant Cell, 2007; incorporated herein by reference in its entirety]. In some embodiments, certain genes associated with endogenous silencing pathways, e.g., “Silencing Genes” can be silenced using gene editing technologies and/or endogenous silencing pathways.

-Exemplary E. aureum RDR6 genomic sequence ()
SEQ ID NO: 96
CTGTGACAACAAAATGGGTTCCCTGGGGTCTGACAAGGACAAGAAGGACTTGATTGTCACTCAA
GTTGGTGTTGGTGGTTTTGGTGACAAGGTTTCAGCAAAAGAGCTAACTGACTTTCTGGAATCTA
AAGTGGGGCTAATATGGAGATGTAGACTGAAGACTTCTTGGACCCCACCAGAATCCTACCCGGA
CTTTCAAGTTGCCATTACATCTGAGACCCTAAGGACAGGTAAATATGAAAAAGTGGTGCCTCAT
GCATTTGTACACTTCGCAGTTTCTGATGGGGCCAAGAGGGCTGTCAATGCTGCTGGCAAATCTG
AGCTCATGTTGAATGGCTGCTGCCTCAAGGTAAACTCAGGGATGGACAGTGCTTTCCGGGTAAA
TCGGAGGAGAACTACAGATCCATTTAAGTTTTCTGATGTCCATGTTGAGATAGGAACTCTATGC
AGTCGGGATGAATTCTGGGTTGGTTGGGAAGGACCTAACTCTGGTGTTGATTTTGTAATTGATC
CTTTTGATGGTTGTTGTAAAATACTTTTCTCAAGGGAGGTGGTGTTCTCATTTAAAGGAAGGAA
AGAGACGGCCGTGCTCAAATGTGATGTCAAGATTGAATTCTTTGTGAGAGAGATCAATGAAATA
AGATTGTATACTGACACGTCACCATTTGTGGTACTATTACATCTTGCCTCCTCTCCTTTAGTCT
ATTATAGAACAGCAGATGATGATATATATGTCTCTGTACCATTCAATTTACTAGATGATGAAGA
CCCATGGATAAGAACAACTGACTTCACCCCCGGTGGAGCCATTGGCAGGTGTAGTTCTTATAGG
ATTTCTCTCTCCCCCCGCTATTGGGCTAAGTTGAAGAAAGCCATGAACTACATGAGGGAACGCA
GGATCATTGAACAGCAGCCTAAGCATGACCTCTTAGTCCTAAAAGAGCCTTCCTATGGATCACC
AACTTTAGATGTGTTTTTCTGCATTGAACATGCCGGTATCAGTTTCAATATTATGTTTTTGGTG
AATGTTTTGGTGCATAAAGGTATTTTCAATCAACATCAGTTGTCTGATGATTTCTTTGCATTGC
TGACAAGACAGAATGGCATTGTAAATGAGGCATCACTGCGGCATATCTGTTCATATAAGCGGCC
CATATTTGATGCTACACGAAGGCTAAAGCTTGTACAGCAATGGTTTCTGAAGAATCCTAAACTA
CTGAAAACGAGTAAGACTTCTGCAGATAATGCTGAAGTAAGGAGGTTGATTATAACGCCTACAA
AGGCATATTGTCTCCCTCCCGAGATCGAACTCTCCAATAGAGTTCTTAGAAAATACAAGGAGGT
TGCTGACAGGTTCTTGAGAGTTACTTTCATGGATGAAGGGATGCAGCAGTTGAATAACAATGTT
CTGACGTACTATTCTGCACCTATTGTTAGGGACATAACTAAGAACTCATACTCTCAGAAGACAA
CTGTGTTTAAAAGGGTGAAGAGTATTTTAACTAATGGTTTTCACTTATGTGGTCGGAAATACTC
CTTTCTTGCTTTCTCATCTAATCAATTGAGGGACAGGTCTGCATGGTTCTTTGCACAGGACAAG
GATCATAATGTCAACTCCATCAGAATTTGGATGGGTAAGTTTTCAAATAGGAACATCGCAAAAT
GTGCTGCTCGGATGGGTCAGTGTTTTTCATCTACATATGCCACAGTGAACGTTCCATCAGAAGA
GGTTGATCCTGAATTTCAAGATATTGAGAGAAATAACTATGTTTTCTCTGATGGTATTGGAAAA
CTGACGCCTGATCTTGCTACAGAAGTTGCTGAAAAATTGCAACTGGCTGATAATCCGCCTTCTG
CCTATCAAATTAGGTATGCTGGTTGCAAGGGTGTTATAGCTGTATGGCCTGGAAATGGCAATGG
AATCCGACTCTTCCTGAGGCCAAGCATGAATAAATTTGAATCACTTCACACTGTACTTGAGGTT
GTGTCATGGACCCGATTCCAACCAGGCTTCCTGAACCGTCAGATTGTAACCTTGCTTTCATCCT
TGGGTGTTGCAGATTCTGTGTTTGATATGATGCAGGATTTGATGATTTGTAAGCTAGACCAGAT
GCTTGTGGACACTGATGTGGCATTTGATGTTCTTACTACATCATGTGCTGAACATGGGAATATT
GCAGCATTAATGCTTAGTGCTGGTTTTAGACCTAAGACTGAGCCACATCTCAAAGGAATGCTCT
CTTGCATAAGGTCTGCCCAACTTGGAGACCTTTTGAGAAAGGCAAGGATCTTCATCCCCAAGGG
ACGTTGGCTGATGGGTTGCTTGGATGAACTAGGTGTACTTGAGCATGGGCAATGCTTTATCCAG
GTATCAACTCCATCATTGGAAAATTACTTCTCAAAACATGGTTCCGGGTTTTCTGAAACTAAGA
AAGTCAGACAAACAATCACCGGGACTGTTGCAATTGCAAAGAACCCTTGTCTTCATCCCGGAGA
TATCAGAATACTAGAAGCAGTTGATGTGCCTGGCCTGCATCATCTTGTTGATTGTTTAGTTTTT
CCTCAAAAGGGTGATAGGCCTCATACAAATGAGGCATCGGGAAGTGACCTGGATGGGGATCTGT
ATTTTGTTACCTGGGATGAGAATCTCTTACCCCCAGGTAAGAAGAGCTGGCCACCAATGGATTA
TGCAGCTCCAGAAGTCAAGCAATTGCCTCGCCCAGTTACTCACACA
-Exemplary E. aureum RDR6 amino acid sequence
SEQ ID NO: 97
MCWWTMGTNQWQQLWACKQQIEASLDADQARVASGQPRTVMTVFRKLLYCDNKMGSLGSDKDKK
DLIVTQVGVGGFGDKVSAKELTDFLESKVGLIWRCRLKTSWTPPESYPDFQVAITSETLRTGKY
EKVVPHAFVHFAVSDGAKRAVNAAGKSELMLNGCCLKVNSGMDSAFRVNRRRTTDPFKFSDVHV
EIGTLCSRDEFWVGWEGPNSGVDFVIDPFDGCCKILFSREVVFSFKGRKETAVLKCDVKIEFFV
REINEIRLYTDTSPFVVLLHLASSPLVYYRTADDDIYVSVPFNLLDDEDPWIRTTDFTPGGAIG
RCSSYRISLSPRYWAKLKKAMNYMRERRIIEQQPKHDLLVLKEPSYGSPTLDVFFCIEHAGISF
NIMFLVNVLVHKGIFNQHQLSDDFFALLTRQNGIVNEASLRHICSYKRPIFDATRRLKLVQQWE
LKNPKLLKTSKTSADNAEVRRLIITPTKAYCLPPEIELSNRVLRKYKEVADRFLRVTFMDEGMQ
QLNNNVLTYYSAPIVRDITKNSYSQKTTVFKRVKSILINGFHLCGRKYSFLAFSSNQLRDRSAW
FFAQDKDHNVNSIRIWMGKFSNRNIAKCAARMGQCFSSTYATVNVPSEEVDPEFQDIERNNYVF
SDGIGKLTPDLATEVAEKLQLADNPPSAYQIRYAGCKGVIAVWPGNGNGIRLFLRPSMNKFESL
HTVLEVVSWTRFQPGFLNRQIVTLLSSLGVADSVFDMMQDLMICKLDQMLVDTDVAFDVLTTSC
AEHGNIAALMLSAGFRPKTEPHLKGMLSCIRSAQLGDLLRKARIFIPKGRWLMGCLDELGVLEH
GQCFIQVSTPSLENYFSKHGSGFSETKKVRQTITGTVAIAKNPCLHPGDIRILEAVDVPGLHHL
VDCLVFPQKGDRPHTNEASGSDLDGDLYFVTWDENLLPPGKKSWPPMDYAAPEVKQLPRPVTHT
DIIDFFTKNMVNESLGVICNGHVVHADRSEQGAMDTKCLLLAELAALAVDFPKTGKIVSMPHDL
KPKLYPDFMGKDDFLSYKSDKILGKLYRKIKDSSEEDGLTSDLSYKHEDIPYDIDLEIGGASHF
LEDAWDRKCSYDTVLNALLGQYRVNSEGEVVTGHIWSMPKFNSHDERGKLYEQKASAWYQVTYH
PQWVKKALDLREPDGDHIPPRLSFAWIPVDYLVRIKVRSRSDKGELDGNKPVDALAAYLRDRV

In some embodiments, a genome editing system targets nucleotides within a specific target site, e.g., within a specific gene. In some such embodiments, a target site is or comprises, but is not limited by, an endogenous loci known to impact: transgene expression, stomatal flux, trichome density, cuticle wax levels, metabolic pathways, or any combination of these pathways.

In some embodiments, a genome editing system comprises a nucleic acid strand that is complementary to a target site in a gene (e.g., complementary to a nucleotide sequence that is at least 85%, 90%, 91%, 92%, 93%, 94%, 95%, 96%, 97%, 98%, 99%, or 100% identical to a portion of SEQ ID NO: 96 or a characteristic portion thereof. In some embodiments, a genome editing system comprises a nucleic acid strand that is complementary to a target site in a gene (e.g., complementary to a nucleotide sequence that is at least 85%, 90%, 91%, 92%, 93%, 94%, 95%, 96%, 97%, 98%, 99%, or 100% identical to a portion of a sequence encoding a protein sequence represented by SEQ ID NO: 97 or a characteristic portion thereof. In some embodiments, a target site may be 15-30 nucleotides long, e.g., 15, 16, 17, 18, 19, 20, 21, 22, 23, 24, 25, 26, 27, 28, 29, or 30 nucleotides long, although shorter and longer target sites are also contemplated.

In some embodiments, a genome editing system comprises a nucleic acid strand that comprises a region that is perfectly complementary to at least 6, 7, 8, 9, 10, 11, 12, 13 14, 15, 16, 17, 18, 19, 20, 21, 22, 23, 24, 25, 26, 27, 28, 29, or 30 consecutive nucleotides of a gene. In some embodiments a genome editing system is an RNA-guided nuclease system. In some embodiments, such an RNA-guided nuclease system is capable of inhibiting expression of one or more target genes and/or their associated mRNA, e.g., EPF1, EPF2, RDR6 listed under NCBI RefSeq accession numbers: NM_127657.4, NM_103147.3, and NM_001339423.1 respectively.

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with gRNA, associate with, and optionally cleave or modify, a target region of a DNA that includes (i) a sequence complementary to a targeting domain of a gRNA and, optionally, (ii) an additional sequence referred to as a “protospacer adjacent motif,” or “PAM,” which is described in greater detail herein and within the public literature.

Naturally occurring CRISPR systems are organized evolutionarily into two classes and five types (Makarova et al. Nat Rev Microbiol. 2011 June; 9 (6): 467-477 (“Makarova”), which is incorporated in its entirety herein by reference), and while genome editing systems of the present disclosure may adapt components of any type or class of naturally occurring CRISPR system, embodiments presented herein are generally adapted from Class 2, and type II or V CRISPR systems. Class 2 systems, which encompass types II and V, are characterized by relatively large, multidomain CRISPR proteins (e.g., Cas9 or Cpf1) and one or more gRNAs (e.g., a crRNA and, optionally, a tracrRNA) that form ribonucleoprotein (RNP) complexes that associate with (i.e., target) and cleave specific loci complementary to a targeting (or spacer) sequence of a crRNA. Genome editing systems according to the present disclosure similarly target and edit cellular DNA sequences, but differ significantly from CRISPR systems occurring in nature. For example, unimolecular gRNAs described herein do not occur in nature, and both gRNAs and CRISPR nucleases according to this disclosure may incorporate any number of non-naturally occurring modifications.

As described herein, it should be noted that a genome editing systems of the present disclosure can be targeted to a single specific nucleotide sequence, or may be targeted to—and capable of editing in parallel-two or more specific nucleotide sequences through use of two or more gRNAs. In some embodiments, use of multiple gRNAs is referred to as “multiplexing.” As described herein, multiplexing can be employed, for example, to target multiple, unrelated target sequences of interest, or to form multiple SSBs or DSBs within a single target domain and, in some cases, to generate specific edits within such target domain. For example, International Patent Publication No. WO 2015/138510 by Maeder et al., which is incorporated in its entirety herein by reference; (“Maeder”) describes a genome editing system for correcting a point mutation (C.2991+1655A to G) in human CEP290 that results in t creation of a cryptic splice site, which in turn reduces or eliminates function of the gene. That genome editing system of Maeder utilizes two gRNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), which is incorporated in its entirety herein by reference. Cotta-Ramusino describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al., which is incorporated in its entirety herein by reference; (“Palestrant”) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111 (10): E924-932, Mar. 11, 2014, which is incorporated in its entirety herein by reference (“Davis”) (describing Alt-HDR); Frit et al. DNA Repair 17 (2014) 81-97, which is incorporated in its entirety herein by reference (“Frit”) (describing Alt-NHEJ); and Iyama and Wilson III, DNA Repair (Amst.) 2013-Aug; 12 (8): 620-636, which is incorporated in its entirety herein by reference (“Iyama”) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; a donor template is incorporated into a target region of cellular DNA that is cleaved by a genome editing system, and can result in a change in a target sequence.

In some embodiments, genome editing systems modify a target sequence, or modify expression of a gene in or near a target sequence, without causing single- or double-strand breaks. For example, a genome editing system may include a CRISPR protein fused to a functional domain that acts on DNA, thereby modifying a target sequence or its expression. As one example, a CRISPR protein can be connected to (e.g., fused to) a cytidine deaminase functional domain, and may operate by generating targeted C-to-A substitutions. Exemplary nuclease/deaminase fusions are described in Komor et al. Nature 533, 420-424 (19 May 2016) (“Komor”), which is incorporated in its entirety herein by reference. In some embodiments, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving a targeted region(s) including, without limitation, mRNA transcription, chromatin remodeling, etc. In some embodiments, a genome editing system may be self-inactivating, as described by Li et al. “A Self-Deleting AAV-CRISPR System for In Vivo Editing” Mol Ther Methods Clin Dev. 2019 Mar. 15; 12:111-122; published online (2018 Dec. 6), the contents of which are hereby incorporated by reference in its entirety.

As the following discussion will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus, etc.) or variation (e.g., full-length vs. truncated or split; naturally-occurring PAM specificity vs. engineered PAM specificity, etc.) of RNA-guided nuclease. In some embodiments, a CRISPR/Cas is derived from a type II CRISPR/Cas system. In some embodiments, a CRISPR/Cas system is derived from a Cas9 protein. A Cas9 protein can be from Streptococcus pyogenes, Streptococcus thermophilus, Staphylococcus aureus, Campylobacter jejuni, or other species. In some embodiments, Cas9 can include: spCas9, Cpf1, CasY, CasX, saCas9, or CjCas9.

Administering bacterial Cas9 in plants presents silencing concerns. Therefore, in some embodiments, a codon-optimized CRISPR system is provided to reduce potential silencing.

A PAM sequence takes its name from its sequential relationship to a “protospacer” sequence that is complementary to gRNA targeting domains (or “spacers”). Together with protospacer sequences, PAM sequences define target regions or sequences for specific RNA-guided nuclease/gRNA combinations. Various RNA-guided nucleases may require different sequential relationships between PAMs and protospacers. In general, Cas9s recognize PAM sequences that are 3′ of a protospacer. Cpf1, on the other hand, generally recognizes PAM sequences that are 5′ of a protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from PAM specificities of reference molecules (for instance, in the case of an engineered RNA-guided nuclease, a reference molecule may be a naturally occurring variant from which an RNA-guided nuclease is derived, or a naturally occurring variant having the greatest amino acid sequence homology to an engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154 (6), 1380-1389 Sep. 12, 2013 (“Ran”)), or that that do not cut at all.

### CRISPR Fusion Proteins

As described herein, in some embodiments, a CRISPR nuclease is part of a fusion protein comprising one or more heterologous protein domains (e.g., about or more than about 1, 2, 3, 4, 5, 6, 7, 8, 9, 10, or more domains in addition to a CRISPR nuclease). A CRISPR nuclease fusion protein may comprise any additional protein sequence, and optionally a linker sequence between any two domains. Examples of protein domains that may be fused to a CRISPR nuclease include, without limitation, epitope tags, reporter gene sequences, and protein domains having one or more of the following activities: methylase activity, demethylase activity, transcription activation activity, deamination activity, transcription repression activity, transcription release factor activity, histone modification activity, RNA cleavage activity and nucleic acid binding activity. Additional domains that may form part of a fusion protein comprising a CRISPR nuclease are described in US20110059502, incorporated herein by reference. In some embodiments, a tagged CRISPR nuclease is used to identify a location of a target sequence. In some embodiments, a CRISPR nuclease that is part of a fusion protein has been engineered to produce only SSBs as described herein. In some embodiments, a CRISPR nuclease that is part of a fusion protein has been engineered to not cut at all as described herein.

### CRISPR Variants

In general, RNA-guided nucleases comprise at least one RNA recognition and/or RNA binding domain. RNA recognition and/or RNA binding domains interact with a guiding RNA. CRISPR/Cas proteins can also comprise nuclease domains (i.e., DNase or RNase domains), DNA binding domains, helicase domains, RNAse domains, protein-protein interaction domains, dimerization domains, as well as other domains. RNA-guided nucleases can be modified to increase nucleic acid binding affinity and/or specificity, alter an enzymatic activity, and/or change another property of a protein. In some embodiments, a CRISPR/Cas-like protein of a fusion protein can be derived from a wild type Cas9 protein or fragment thereof. In other embodiments, a CRISPR/Cas can be derived from modified Cas9 protein. For example, an amino acid sequence of a Cas9 protein can be modified to alter one or more properties (e.g., nuclease activity, affinity, stability, and so forth) of a protein. Alternatively, domains of a Cas9 protein not involved in RNA-guided cleavage can be eliminated from a protein such that a modified Cas9 protein is smaller than a wild type Cas9 protein. In general, a Cas9 protein comprises at least two nuclease (i.e., DNase) domains. For example, a Cas9 protein can comprise a RuvC-like nuclease domain and a HNH-like nuclease domain. RuvC and HNH domains work together to cut single strands to make a double-stranded break in DNA (Jinek et al., 2012, Science, 337:816-821, which is incorporated in its entirety herein by reference).

In some embodiments, a Cas9-derived protein can be modified to contain only one functional nuclease domain (either a RuvC-like or a HNH-like nuclease domain). For example, a Cas9-derived protein can be modified such that one nuclease domain is deleted or mutated such that it is no longer functional (i.e., nuclease activity is absent). In some embodiments in which one nuclease domains is inactive, a Cas9-derived protein is able to introduce a nick into a double-stranded nucleic acid (such protein is termed a “nickase”), but not cleave double-stranded DNA. In any of the above-described embodiments, any or all of nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

One example of a CRISPR/Cas9 system used to inhibit gene expression, CRISPRi, is described in U.S. Publication No. US2014/0068797, which is incorporated herein by reference in its entirety. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a gRNA, a DNA recognition complex is generated that specifically interferes with transcriptional elongation, RNA polymerase binding, or transcription factor binding. This CRISPRi system efficiently represses expression of targeted genes.

Guide RNAs (gRNAs)

A gRNA sequence may be specific for any gene, such as a gene that would affect (e.g., improve, attenuate, inhibit) functions related to phytoremediation. In some embodiments, a gene encodes an ion channel subunit. In some embodiments, a gene encodes an enzymatic subunit. In some embodiments, a gene encodes a structural protein subunit. In some embodiments, a gRNA sequence includes an RNA sequence, a DNA sequence, a combination thereof (a RNA-DNA combination sequence), or a sequence with synthetic nucleotides. A gRNA sequence can be a single molecule or a double molecule. In one embodiment, a gRNA sequence comprises a single guide RNA (sgRNA).

In some embodiments, a gRNA sequence is specific for a gene and targets that gene for Cas endonuclease-induced double strand breaks. A sequence of a gRNA may be within a loci of the gene. In one embodiment, a gRNA sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length. In some embodiments, a gRNA sequence is from about 18 to about 22 nucleotides in length.

As described herein, in some embodiments in the context of formation of a CRISPR complex, “target sequence” refers to a sequence to which a guide sequence is designed to have some complementarity, where hybridization between a target sequence and a guide sequence promotes formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In other embodiments, a target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) a target sequence. As with a target sequence, it is believed that complete complementarity is not needed, provided this is sufficient to be functional. In some embodiments, a tracr sequence has at least 50%, 60%, 70%, 80%, 90%, 95% or 99% of sequence complementarity along the length of a tracr mate sequence when optimally aligned.

gRNA Design

Methods for selection and validation of target sequences as well as off-target analyses have been described previously, e.g., in Mali; Hsu; Fu et al., 2014 Nat biotechnol 32 (3): 279-84, Heigwer et al., 2014 Nat methods 11 (2): 122-3; Bae et al. (2014) Bioinformatics 30 (10): 1473-5; and Xiao A et al. (2014) Bioinformatics 30 (8): 1180-1182, each of which is incorporated in its entirety herein by reference. As a non-limiting example, gRNA design may involve use of a software tool to optimize choice of potential target sequences corresponding to a user's target sequence, e.g., to minimize total off-target activity across a genome. While off-target activity is not limited to cleavage, cleavage efficiency at each off-target sequence can be predicted, e.g., using an experimentally-derived weighting scheme. These and other guide selection methods are described in detail in Maeder and Cotta-Ramusino.

For example, in certain embodiments, methods for selection and validation of target sequences in plants as well as off-target analyses can be performed using CRISPR-P, CRISPR-PLANT, and/or CRISPR-GE (Liu et al., CRISPR-P 2.0: An improved CRISPR-Cas9 Tool for Genome Editing in Plants. Mol Plant. 2017 Mar. 6; 10 (3): 530-532; Xie et al., Genome-wide prediction of highly specific guide RNA spacers for CRISPR-Cas9-mediated genome editing in model plants and major crops. Mol Plant. 2014 May 7; (5): 923-6; and Xie et al., CRISPR-GE: A Convenient Software Toolkit for CRISPR-Based Genome Editing. Mol Plant. 2017 Sep. 12; 10 (9): 1246-1249; each of which is incorporated in its entirety herein by reference).

gRNA Modifications

Activity, stability, or other characteristics of gRNAs can be altered through incorporation of certain modifications. As one example, transiently expressed or delivered nucleic acids can be prone to degradation by, e.g., cellular nucleases. Accordingly, gRNAs described herein can contain one or more modified nucleosides or nucleotides that can introduce stability toward nucleases. While not wishing to be bound by theory, it is also believed that certain modified gRNAs described herein can potentially exhibit a reduced silencing response when introduced into plant cells. Those of skill in the art will be aware of certain cellular responses commonly observed in cells, e.g., plant cells, in response to exogenous nucleic acids, particularly those of viral or bacterial origin. Such responses, may potentially be reduced or eliminated altogether by modifications presented herein.

Certain exemplary modifications discussed in this section can be included at any position within a gRNA sequence including, without limitation at or near its 5′ end (e.g., within 1-10, 1-5, or 1-2 nucleotides of a 5′ end) and/or at or near its 3′ end (e.g., within 1-10, 1-5, or 1-2 nucleotides of a 3′ end). In some cases, modifications are positioned within functional motifs, such as a repeat-anti-repeat duplex of a Cas9 gRNA, a stem loop structure of a Cas9 or Cpf1 gRNA, and/or a targeting domain of a gRNA. Others types of modified nucleobases are described herein.

The present disclosure provides technologies (e.g., comprising compositions) that may, in some embodiments, reduce, suppress or otherwise decrease (“knock down”) expression of one or more gene products. For example, in some embodiments, technologies of the present disclosure may achieve knockdown of a EPF1, EPF2, and/or RDR6 gene product (e.g., a gene, mRNA, protein, etc.).

In some embodiments, knockdown of a gene product (e.g., a gene, mRNA, protein, etc.) is achieved using one or more techniques to inhibit one or more gene products or processes by which gene products are produced. For example, in some embodiments, the present disclosure provides technologies that comprise compositions that are or comprise inhibitory nucleic acid molecules to knock down expression of a gene product.

In some embodiments, an inhibitory nucleic acid molecule targets nucleotides within a EPF1, EPF2, and/or RDR6 gene product. In some embodiments, an inhibitory nucleic acid molecule comprises a nucleic acid strand that is complementary to a target site of a gene product, e.g., EPF1, EPF2, and/or RDR6 mRNA (e.g., complementary to a nucleotide sequence that is at least 85%, 90%, 91%, 92%, 93%, 94%, 95%, 96%, 97%, 98%, 99%, or 100% identical to a portion of such a gene). In some embodiments, a target site may be 15-30 nucleotides long, e.g., 15, 16, 17, 18, 19, 20, 21, 22, 23, 24, 25, 26, 27, 28, 29, or 30 nucleotides long, although shorter and longer target sites are also contemplated.

In some embodiments, an inhibitory nucleic acid molecule comprises a nucleic acid strand that comprises a region that is perfectly complementary to at least 6, 7, 8, 9, 10, 11, 12, 13 14, 15, 16, 17, 18, 19, 20, 21, 22, 23, 24, 25, 26, 27, 28, 29, or 30 consecutive nucleotides of a gene of interest or characteristic portions thereof).

In some embodiments an inhibitory nucleic acid molecule is capable of inhibiting expression of a gene product of one or more plant species, e.g., a. In some embodiments, an inhibitory RNA molecule or Genome editing system is complementary to a target portion that is identical in multiple plant species. In some embodiments, an inhibitory RNA molecule is complementary to a target site of one plant species that varies by 1, 2, 3, 4, 5, 6, 7, 8, 9, or 10 nucleotides from another plant species.

### Inhibitory Nucleic Acid Molecules

RNA interference (RNAi) is a process of sequence-specific post-transcriptional gene silencing by which, e.g., double stranded RNA (dsRNA) homologous to a target locus can specifically inactivate gene function (Hammond et al., Nature Genet. 2001; 2:110-119; Sharp, Genes Dev. 1999; 13:139-141). In some embodiments, dsRNA-induced gene silencing can be mediated by short double-stranded small interfering RNAs (siRNAs) generated from longer dsRNAs by ribonuclease III cleavage (Bernstein et al., Nature 2001; 409:363-366 and Elbashir et al., Genes Dev. 2001; 15:188-200). Without being bound by any particular theory, RNAi-mediated gene silencing is thought to occur via sequence-specific RNA degradation and/or sequestration, where sequence specificity is determined by interaction of a siRNA with its complementary sequence within a target RNA (see, e.g., Tuschl, Chem. Biochem. 2001; 2:239-245). In some embodiments, RNAi can involve use of, e.g., siRNAs (Elbashir, et al., Nature 2001; 411:494-498, which is incorporated in its entirety herein by reference) or short hairpin RNAs (shRNAs) bearing a fold back stem-loop structure (Paddison et al., Genes Dev. 2002; 16:948-958; Sui et al., Proc. Natl. Acad. Sci. USA 2002; 99:5515-5520; Brummelkamp et al., Science 2002; 296:550-553; Paul et al., Nature Biotechnol. 2002; 20:505-508, each of which is incorporated in its entirety herein by reference).

In some embodiments an inhibitory nucleic acid is one or more of a short interfering RNA (siRNA), a short hairpin RNA (shRNA), an antisense oligonucleotide, or a ribozyme. In some embodiments, knockdown of a gene of interests expression is achieved via inhibitory nucleic acids that target a gene of interest sequence as described herein. In some such embodiments, a targeted sequence may be a wild-type and/or variant gene sequence.

In some embodiments, an inhibitory nucleic acid of the present disclosure may be used to decrease expression of a gene product. In some such embodiments, a vector encodes an inhibitory nucleic acid that may, in some embodiments, decrease expression of a gene product, e.g., in a plant cell (e.g., a leaf cell, petiole cell, vasculature cell, stem cell, and/or root cell). In some embodiments, after an inhibitory nucleic acid is used to decrease expression of a gene product, another (i.e., non-inhibitory) nucleic acid molecule may be used to express a functional protein of interest.

siRNA or shRNA

In some embodiments, the present disclosure provides an inhibitory nucleic acid, e.g., a chemically-modified siRNAs or a vector-driven expression of short hairpin RNA (shRNA) that are then cleaved to siRNA, e.g., within a cell. Accordingly, one of skill in the art will understand that, for purposes of sequences, an shRNA sequence is interchangeable with an siRNA sequence and that where the disclosure refers to an siRNA, an shRNA sequence may be used since the shRNA will be cleaved into siRNA. For example, in some embodiments, an inhibitory nucleic acid can be a dsRNA (e.g., siRNA) including 16-30 nucleotides, e.g., 16, 17, 18, 19, 20, 21, 22, 23, 24, 25, 26, 27, 28, 29, or 30 nucleotides in each strand, where one strand is substantially identical, e.g., at least 80% (or more, e.g., 85%, 90%, 95%, or 100%) identical, e.g., having 3, 2, 1, or 0 mismatched nucleotide(s), to a target region in a gene, and the other strand is complementary to the first strand. In some embodiments, dsRNA molecules can be designed using methods known in the art, e.g., Dharmacon.com (see, siDESIGN CENTER) or “The siRNA User Guide,” available on the Internet at mpibpc.gwdg.de/abteilungen/100/105/sirna.html website which is incorporated in its entirety herein by reference. Without being bound by any particular theory, the present disclosure contemplates that siRNA or shRNAs are more “endogenous” (e.g., no foreign proteins) in a way that may be more recognizable to a cell compared to other available techniques that will be known to those of skill in the art. Accordingly, in some embodiments, siRNA or shRNA have lower inhibitory silencing potential and/or have less risk of off-target DNA interaction as compared to other techniques known to those of skill in the art.

In some embodiments, siRNAs of the present disclosure are double stranded nucleic acid duplexes (of, e.g., 15, 16, 17, 18, 19, 20, 21, 22, 23, 24, 25, 26, or 27 base pairs) comprising annealed complementary single stranded nucleic acid molecules. In some embodiments, siRNAs are short dsRNAs comprising annealed complementary single strand RNAs. In some embodiments, siRNAs comprise an annealed RNA: DNA duplex, wherein the sense strand of a duplex is a DNA molecule and the antisense strand of the same duplex is a RNA molecule. In some embodiments, duplexed siRNAs comprise a 2 or 3 nucleotide 3′ overhang on each strand of a duplex. In some embodiments, siRNAs comprise 5′-phosphate and 3′-hydroxyl groups.

In some embodiments, a siRNA molecule of the present disclosure includes one or more natural nucleobase and/or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being natural degradation products). Exemplary modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313, each of which is incorporated in its entirety herein by reference.

Modified nucleobases also include expanded-size nucleobases in which one or more aryl rings, such as phenyl rings, have been added. Nucleic base replacements described in the Glen Research catalog (available on the world wide web at glenresearch.com); Krueger A T et al., Acc. Chem. Res., 2007, 40, 141-150; Kool, ET, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, each of which is incorporated in its entirety herein by reference, are contemplated as useful for siRNA molecules described herein. In some embodiments, modified nucleobases also encompass structures that are not considered nucleobases but are other moieties such as, but not limited to, corrin- or porphyrin-derived rings. Porphyrin-derived base replacements have been described in Morales-Rojas, H and Kool, ET, Org. Lett., 2002, 4, 4377-4380, which is incorporated in its entirety herein by reference.

In some embodiments, modified nucleobases are of any one of the following structures, optionally substituted:

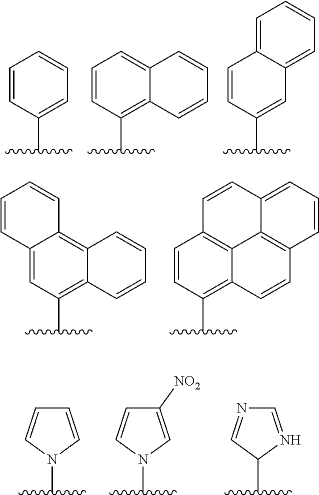

In some embodiments, a modified nucleobase is fluorescent. Exemplary such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, and naphtho-uracil.

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, siRNA molecules described herein include nucleosides that incorporate modified nucleobases and/or nucleobases covalently bound to modified sugars. Some examples of nucleosides that incorporate modified nucleobases include 4-acetylcytidine; 5-(carboxyhydroxylmethyl) uridine; 2′-O-methylcytidine; 5-carboxymethylaminomethyl-2-thiouridine; 5-carboxymethylaminomethyluridine; dihydrouridine; 2′-O-methylpseudouridine; beta, D-galactosylqueosine; 2′-O-methylguanosine; N6-isopentenyladenosine; 1-methyladenosine; 1-methylpseudouridine; 1-methylguanosine; 1-methylinosine; 2,2-dimethylguanosine; 2-methyladenosine; 2-methylguanosine; N7-methylguanosine; 3-methyl-cytidine; 5-methylcytidine; 5-hydroxymethylcytidine; 5-formylcytosine; 5-carboxylcytosine; N°-methyladenosine; 7-methylguanosine; 5-methylaminoethyluridine; 5-methoxyaminomethyl-2-thiouridine; beta, D-mannosylqueosine; 5-methoxycarbonylmethyluridine; 5-methoxyuridine; 2-methylthio-N6-isopentenyladenosine; N-((9-beta, D-ribofuranosyl-2-methylthiopurine-6-yl) carbamoyl) threonine; N-((9-beta, D-ribofuranosylpurine-6-yl)-N-methylcarbamoyl) threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, nucleosides include 6′-modified bicyclic nucleoside analogs that have either (R) or(S)-chirality at the 6′-position and include the analogs described in U.S. Pat. No. 7,399,845, which is incorporated in its entirety herein by reference. In other embodiments, nucleosides include 5′-modified bicyclic nucleoside analogs that have either (R) or (S)-chirality at the 5′-position and include the analogs described in U.S. Publ. No. 20070287831, which is incorporated in its entirety herein by reference. In some embodiments, a nucleobase or modified nucleobase is 5-bromouracil, 5-iodouracil, or 2,6-diaminopurine. In some embodiments, a nucleobase or modified nucleobase is modified by substitution with a fluorescent moiety.

Methods of preparing modified nucleobases are described in, e.g., U.S. Pat. Nos. 3,687,808; 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is incorporated in its entirety herein by reference.

In some embodiments, a siRNA molecule described herein includes one or more modified nucleotides wherein a phosphate group or linkage phosphorus in its nucleotides are linked to various positions of a sugar or modified sugar. As non-limiting examples, a phosphate group or linkage phosphorus can be linked to a 2′, 3′, 4′ or 5′ hydroxyl moiety of a sugar or modified sugar. Nucleotides that incorporate modified nucleobases as described herein are also contemplated in this context.

Other modified sugars can also be incorporated within a siRNA molecule. In some embodiments, a modified sugar contains one or more substituents at a 2′ position including one of the following: —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. Examples of substituents include, and are not limited to, —O(CH₂)ₙOCH₃, and —O(CH₂)ₙNH₂, wherein n is from 1 to about 10, MOE, DMAOE, DMAEOE. Also contemplated herein are modified sugars described in WO 2001/088198; and Martin et al., Helv. Chim. Acta, 1995, 78, 486-504, each of which is incorporated in its entirety herein by reference. In some embodiments, a modified sugar comprises one or more groups selected from a substituted silyl group, an RNA cleaving group, a reporter group, a fluorescent label, an intercalator, a group for improving pharmacokinetic properties of a nucleic acid, a group for improving pharmacodynamic properties of a nucleic acid, or other substituents having similar properties. In some embodiments, modifications are made at one or more of a 2′, 3′, 4′, 5′, or 6′ positions of a sugar or modified sugar, including a 3′ position of a sugar on a 3′-terminal nucleotide or in a 5′ position of a 5′-terminal nucleotide.

In some embodiments, a 2′-OH of a ribose is replaced with a substituent including one of the following: —H, —F; —CF₃, —CN, —N₃, —NO, —NO₂, —OR′, —SR′, or —N(R′)₂, wherein each R′ is independently as defined above and described herein; —O—(C₁-C₁₀ alkyl), —S(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein an alkyl, alkylene, alkenyl and alkynyl may be substituted or unsubstituted. In some embodiments, a 2′-OH is replaced with —H (deoxyribose). In some embodiments, a 2′-OH is replaced with —F. In some embodiments, a 2′-OH is replaced with —OR′. In some embodiments, a 2′-OH is replaced with —OMe. In some embodiments, a 2′-OH is replaced with —OCH₂CH₂OMe.

Modified sugars also include locked nucleic acids (LNAs). In some embodiments, a locked nucleic acid has the structure indicated below. A locked nucleic acid of the structure below is indicated, wherein Ba represents a nucleobase or modified nucleobase as described herein, and wherein R^{2s} is —OCH₂C4′-

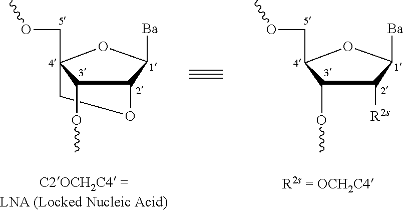

In some embodiments, a modified sugar is an ENA such as those described in, e.g., Seth et al., J Am Chem Soc. 2010 Oct. 27; 132 (42): 14942-14950, which is incorporated in its entirety herein by reference. In some embodiments, a modified sugar is any of those found in an XNA (xenonucleic acid), for instance, arabinose, anhydrohexitol, threose, 2′fluoroarabinose, or cyclohexene.

Modified sugars include sugar mimetics such as cyclobutyl or cyclopentyl moieties in place of the pentofuranosyl sugar (see, e.g., U.S. Pat. Nos. 4,981,957; 5,118,800; 5,319,080; and 5,359,044, each of which is incorporated in its entirety herein by reference). Some modified sugars that are contemplated include sugars in which an oxygen atom within a ribose ring is replaced by nitrogen, sulfur, selenium, or carbon. In some embodiments, a modified sugar is a modified ribose wherein an oxygen atom within a ribose ring is replaced with nitrogen, and wherein a nitrogen is optionally substituted with an alkyl group (e.g., methyl, ethyl, isopropyl, etc.).

Non-limiting examples of modified sugars include glycerol, which form glycerol nucleic acid (GNA) analogues. An exemplary GNA analogue is described in Zhang, R et al., J. Am. Chem. Soc., 2008, 130, 5846-5847, which is incorporated in its entirety herein by reference; see also Zhang L, et al., J. Am. Chem. Soc., 2005, 127, 4174-4175 and Tsai C H et al., PNAS, 2007, 14598-14603, each which is incorporated in its entirety herein by reference. Another example of a GNA derived analogue, flexible nucleic acid (FNA) based on mixed acetal aminal of formyl glycerol, is described in each of Joyce G F et al., PNAS, 1987, 84, 4398-4402 and Heuberger B D and Switzer C, J. Am. Chem. Soc., 2008, 130, 412-413, each of which is incorporated in its entirety herein by reference. Additional non-limiting examples of modified sugars include hexopyranosyl (6′ to 4′), pentopyranosyl (4′ to 2′), pentopyranosyl (4′ to 3′), or tetrofuranosyl (3′ to 2′) sugars.

Modified sugars and sugar mimetics can be prepared by methods known in the art, including, but not limited to: A. Eschenmoser, Science (1999), 284:2118; M. Bohringer et al., Helv. Chim. Acta (1992), 75:1416-1477; M. Egli et al., J. Am. Chem. Soc. (2006), 128 (33): 10847-56; A. Eschenmoser in Chemical Synthesis: Gnosis to Prognosis, C. Chatgilialoglu and V. Sniekus, Ed., (Kluwer Academic, Netherlands, 1996), p.293; K. U. Schoning et al., Science (2000), 290:1347-1351; A. Eschenmoser et al., Helv. Chim. Acta (1992), 75:218; J. Hunziker et al., Helv. Chim. Acta (1993), 76:259; G. Otting et al., Helv. Chim. Acta (1993), 76:2701; K. Groebke et al., Helv. Chim. Acta (1998), 81:375; and A. Eschenmoser, Science (1999), 284:2118. Modifications to 2′ modifications can be found in Verma, S. et al. Annu. Rev. Biochem. 1998, 67, 99-134 and all references therein, each of which is incorporated in its entirety herein by reference. Specific modifications to a ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310); PCT Publication No. WO2012/030683, each of which is incorporated in its entirety herein by reference.

In some embodiments, a siRNA described herein can be introduced to a target cell as an annealed duplex siRNA. In some embodiments, a siRNA described herein is introduced to a target cell as single stranded sense and antisense nucleic acid sequences that, once within a target cell, anneal to form a siRNA duplex. Alternatively, sense and antisense strands of an siRNA can be encoded by an expression vector (such as an expression vector described herein) that is introduced to a target cell. Upon expression within a target cell, transcribed sense and antisense strands can anneal to reconstitute an siRNA.

In some embodiments, an siRNA molecule as described herein can be synthesized by standard methods known in the art, e.g., by use of an automated synthesizer. Without being bound by any particular theory, RNAs produced by such methodologies tend to be highly pure and to anneal efficiently to form siRNA duplexes. In some embodiments, following chemical synthesis, single stranded RNA molecules can be deprotected, annealed to form siRNAs, and purified (e.g., by gel electrophoresis or HPLC). Alternatively, in some embodiments, standard procedures can be used for in vitro transcription of RNA from DNA templates, e.g., carrying one or more RNA polymerase promoter sequences (e.g., T7 or SP6 RNA polymerase promoter sequences). Protocols for preparation of siRNAs using T7 RNA polymerase are known in the art (see, e.g., Donze and Picard, Nucleic Acids Res. 2002; 30: e46; and Yu et al., Proc. Natl. Acad. Sci. USA 2002; 99:6047-6052, each of which is incorporated in its entirety herein by reference). In some embodiments, sense and antisense transcripts can be synthesized in two independent reactions and annealed later. In some embodiments, sense and antisense transcripts can be synthesized simultaneously in a single reaction.

In some embodiments, an siRNA molecule can also be formed within a cell by transcription of RNA from an expression vector introduced into a cell (see, e.g., Yu et al., Proc. Natl. Acad. Sci. USA 2002; 99:6047-6052, which is incorporated in its entirety herein by reference). For example, in some embodiments, an expression vector for in vivo production of siRNA molecules can include one or more siRNA encoding sequences operably linked to elements necessary for proper transcription of an siRNA encoding sequence(s), including, e.g., promoter elements and transcription termination signals. In some embodiments, preferred promoters for use in such expression vectors may include, e.g., a polymerase-II or polymerase-III promoter, (see, e.g., Wang et al., RNA; 14 (5): 903-913, 2008, which is incorporated in its entirety herein by reference), a U6 polymerase-III promoter (see, e.g., Sui et al., Proc. Natl. Acad. Sci. USA 2002; Paul et al., Nature Biotechnol. 2002; 20:505-508; and Yu et al., Proc. Natl. Acad. Sci. USA 2002; 99:6047-6052, each of which is incorporated in its entirety herein by reference). In some embodiments, an siRNA expression vector can comprise one or more vector sequences that facilitate cloning of an expression vector.

In some embodiments, an siRNA comprises a mature guide strand having a nucleotide sequence that is at least 90%, 91%, 92%, 93%, 94%, 95%, 96%, 97%, 98%, 99%, or 100% identical to a portion of a target gene. In some embodiments, a portion is 15, 16, 17, 18, 19, or 20 nucleotides long. In some embodiments, the present disclosure provides shRNA sequences, which, when introduced into a cell will be cleaved to siRNAs.

miRNA

The present disclosure provides technologies related to or comprising one or more inhibitory nucleic acid molecules such as, e.g., one or more nucleotide sequences that are, comprise, or encode, microRNAs. MicroRNAs (miRNAs) are a highly conserved class of small RNA molecules that are transcribed from DNA in genomes of plants and animals, but are not translated into protein. As is known to those in the art, plant cells express a range of noncoding RNAs of approximately 21 or 22 nucleotides termed micro RNA (miRNAs) and can regulate gene expression at a post transcriptional or translational level during plant development. miRNAs are excised from an approximately 60-500 nucleotide stem-loop primary miRNA transcripts (pri-miRNA). By substituting stem sequences of an miRNA precursor with miRNA sequence complementary to a target mRNA, a vector that expresses a novel miRNA can be used to produce siRNAs to initiate RNAi against specific mRNA targets in plant cell (see e.g., Wang et al., Frontiers in Plant Science, 2019, which is incorporated herein in its entirety by reference). In some embodiments, when expressed by DNA vectors containing polymerase II promoters, micro-RNA designed hairpins can silence gene expression.

In some embodiments, miRNAs can be synthesized and locally or systemically administered to a subject cell and/or tissue, e.g., for gene regulatory purposes. In some embodiments, miRNAs can be designed and/or synthesized as mature molecules or precursors (e.g., pri- or pre-miRNAs). In some embodiments, a pre-miRNA includes a guide strand and a passenger strand that are the same length (e.g., about 15, 16, 17, 18, 19, 20, 21, 22, 23, 24, or 25 nucleotides). In some embodiments, a pre-miRNA includes a guide strand and a passenger strand that are different lengths (e.g., one strand is about 19 nucleotides, and the other is about 21 nucleotides). In some embodiments, an miRNA can target a coding region, a 5′ untranslated region, and/or a 3′ untranslated region, of endogenous mRNA. In some embodiments, an miRNA comprises a guide strand comprising a nucleotide sequence having sufficient sequence complementary with an endogenous mRNA of a subject to hybridize with and inhibit expression of endogenous mRNA.

In some embodiments, miRNAs has advantages compared to shRNAs for inhibiting nucleic acids. For example, in some embodiments, shRNA requires a high level of expression, can clog Argonaut machinery, is not endogenous, and potentially relies upon multiple promoters. By contrast, in some embodiments, it is contemplated that miRNA is more “endogenous” than shRNA, and therefore, is expressed at more endogenous levels that may be handled more readily by the cells endogenous RNA processing machinery. That is, in some embodiments, miRNAs can be synthetic or naturally occurring and naturally-occurring miRNAs are present in cells across plant species.

### Antisense Nucleic Acid

In some embodiments, an inhibitory nucleic acid molecule may be or comprise an antisense nucleic acid molecule, e.g., nucleic acid molecules whose nucleotide sequence is complementary to all or part of a target gene. In some embodiments, an antisense nucleic acid molecule can be antisense to all or part of a non-coding region of a coding strand of a nucleotide sequence of a target gene. In some embodiments, a non-coding regions (“5′ and 3′ untranslated regions”) are 5′ and 3′ sequences that flank a coding region and are not translated into amino acids. Based upon sequences disclosed herein, one of skill in the art can choose and synthesize any of a number of appropriate antisense molecules to target a gene of interest as described herein. For example, a “gene walk” comprising a series of oligonucleotides of 15-30 nucleotides spanning a length of a nucleic acid (e.g., of a gene of interest) can be prepared, followed by testing for inhibition of expression of the target gene. Optionally, gaps of 5-10 nucleotides can be left between oligonucleotides to reduce numbers of oligonucleotides synthesized and tested.

In some embodiments, an antisense oligonucleotide can be, for example, about 5, 10, 15, 20, 25, 30, 35, 40, 45, or 50 nucleotides or more in length. One of skill in the art will recognize that an antisense oligonucleotide can be synthesized using various different chemistries.

### Ribozymes

In some embodiments, an inhibitory nucleic acid molecule may be or comprise a ribozyme. As is known to those of skill in the art, ribozymes are catalytic RNA molecules with ribonuclease activity. In some embodiments, a ribozyme may be used as a controllable promoter. In some embodiments, ribozymes are capable of cleaving a single-stranded nucleic acid, such as an mRNA, to which they have a complementary region. Thus, in some embodiments, ribozymes (e.g., hammerhead ribozymes (described in Haselhoff and Gerlach, Nature, 334:585-591, 1988, which is incorporated in its entirety herein by reference)) can be used to catalytically cleave mRNA transcripts to thereby inhibit translation of a protein encoded by a given mRNA. Methods of designing and producing ribozymes are known in the art (see, e.g., Scanlon, 1999, Therapeutic Applications of Ribozymes, Humana Press, which is incorporated in its entirety herein by reference). In some embodiments, for example, a ribozyme having specificity for a gene of interest can be designed based upon a known nucleotide sequence. For example, a derivative of a Tetrahymena L-19 IVS RNA can be constructed in which nucleotide sequence of an active site is complementary to a nucleotide sequence to be cleaved in a target gene mRNA product (Cech et al. U.S. Pat. No. 4,987,071; and Cech et al., U.S. Pat. No. 5,116,742, each of which is incorporated in its entirety herein by reference). Alternatively, an mRNA encoding a target gene product protein can be used to select a catalytic RNA having a specific ribonuclease activity from a pool of RNA molecules (See, e.g., Bartel and Szostak, Science, 261:1411-1418, 1993, which is incorporated in its entirety herein by reference).

### Enzyme Optimization

The present disclosure recognizes that in certain embodiments, technologies described herein comprising specific metabolic pathways may require optimization to facilitate effective VOC uptake and/or metabolism.

In some embodiments, technologies described herein comprising specific metabolic pathways comprise nucleotide coding sequences that have been codon optimized for their respective host organism.

In some embodiments, synthetic pathways are utilized to increase VOC uptake and/or metabolism. In some embodiments, these synthetic pathways comprise enzymes that have been optimized to catalyze their reactions at as fast a rate as biologically feasible. In some embodiments, this is done by the overexpression of proteins, and/or by altering the structure of the enzymes expressed. In some embodiments, the catalytic activity of a protein can be greatly enhanced by point mutations, deletions, rearrangements (a process often called directed mutagenesis). Furthermore, in some embodiments, the activity (or flux) of certain pathways can be increased by the fusion of the coding sequences of genes constituting that pathway.

### Directed Mutagenesis

In some embodiments, to increase the activity of a given enzyme, specific mutations are induced, typically leading to a change in its catalytic site, (e.g., the active site often considered crucial for its enzymatic reaction). In some embodiments, these mutations can be deliberately chosen through careful examination of the protein structure and activity, sometimes called evolution by rational design. Alternatively, in some embodiments, the mutations can also be random, driven through a process called directed evolution; wherein random mutations are introduced with multiple rounds of error-prone amplification of the DNA sequence. In some embodiments, such amplification of a DNA sequence may occur through a system such as error-prone polymerase chain reaction. In some embodiments, such amplification of a DNA sequence may occur through introduction of the gene into a mutagenic vector and/or organism (e.g., XL1 Red). Those skilled in the art will recognize there are multiple suitable methods for mediating error-prone DNA amplification. In some embodiments, this methodology results in a mutant library from which we can test the activity and select the most active and/or desirable variants from the pool of available mutants. This process allows the testing of many thousands of iterations in parallel, coupling the power of error-prone amplification with stringent selection to harness directed evolution and to create desired and yet difficult to predict mutant enzymes.

### Fusion and Chimeric Proteins

In some embodiments, sequences of individual genes of interest coding for enzymes of interest are optimized through the addition of heterologous protein domains, wherein domains are combined to create “fusion proteins”. In some embodiments, instead of inserting at least two genes, each with its own promoter, coding for at least two enzymes involved in the same or related pathways, a single coding sequence can be inserted. In some embodiments, that sequence comprises the first gene sequences without its stop codon, an optional linker region (e.g., a string of 10-12 codons coding for neutral amino acids), followed by the coding sequence of at least a second gene of interest, wherein the final coding sequence comprises a stop codon. In some embodiments, this method can result in a single reading frame and the expression of a single fusion protein. In some embodiments, this methodology provides certain advantages, e.g., a fusion protein comprising at least two proteins may bring their respective catalytic sites into closer physical proximity, increasing the overall reaction speed. In some embodiments, this method can be used to create fusion proteins combining 3 or more proteins (e.g., at least 3 proteins, at least 4 proteins, at least 5 proteins, at least 6 proteins), however, this may induce steric hindrance. Therefore, in some embodiments, when possible, pairs of proteins involved in the same pathway (e.g., HPS and PHI) are fused together.

Effects of Engineering on Ornamental Plants and/or Microbes
Increasing Diffusion and/or Active Transport

Among other things, the present disclosure provides compositions, methods of producing, and methods of using genetically modified plants with increased diffusion and/or active transport components.

In some embodiments, compositions as described herein may include a passive or an active bio filtering system.

In some embodiments, provided herein are compositions and methods that utilize genetically modified plants alone or in combination with a modified microbiome and/or active or non-active air flow system. In some embodiments, a composition described herein may have an optimized passive and/or active biofiltration phenotype (i.e. passive or active diffusion). In some embodiments, a composition or method described herein comprises a modified plant in combination with a non-active airflow system (e.g., a standard container, e.g., a pot). In some embodiments, compositions and methods described herein comprise a genetically modified plant and an active airflow system that increases airflow to and/or around a plant. In some embodiments, an active airflow system solves a potential problem of air stagnation, e.g., in some embodiments, compositions as described herein are placed inside a container (e.g., planting pot) that generates an airflow directed towards the composition (e.g., soil, leaves, and/or stems, e.g., plant tissue and/or microbiome comprising compositions). In some embodiments, an active airflow promotes air circulation within a room and promotes passage of pollutant particles onto and/or into a plant and/or associated microbes. In some embodiments, such an active system increases the effectiveness of the system e.g., 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, or greater than 10 fold when compared to a control system.

In some embodiments, compositions described herein have an increased rate of diffusion when compared to an appropriate control. In some embodiments, an increased rate in diffusion may be due to an increase in stomatal flux. In some embodiments, an increase in stomatal flux may be due to an increase in total stomata number and/or density.

### Increasing Stomatal Flux

Stomata are microscopic structures located on the plant epidermis, consisting of a pair of guard cells acting as a valve that generates a central pore, providing access to air for mesophyll cells. Stomata act as the main gateway through which gasses, including indoor air pollutants, enter the interior of the plant. In some embodiments, to increase pollution absorption by a plant, stomatal conductance is modified. In some embodiments, stomatal conductance is increased relative to a control. In some embodiments, stomatal conductance is determined by stomatal density and stomatal aperture size.

In some embodiments, the present disclosure provides compositions and methods suitable for increasing and/or otherwise modifying the rate of stomatal conductance (e.g., passive or active diffusion rates of certain volatile compounds). In some embodiments, stomatal conductance is modified through the transgenic expression of genes associated with the positive regulation of stomatal density. In some embodiments, stomatal conductance is modified through the transgenic expression of an EPFL9 gene. In some embodiments, stomatal conductance is increased through the transgenic overexpression of an EPFL9 gene.

In some embodiments, stomatal flux is modified through the transgenic mediated downregulation of genes associated with the negative regulation of stomatal density. In some embodiments, stomatal conductance is modified by downregulation of Epidermal Patterning Factors Like proteins (e.g., EPFL1 and/or EPFL2) that are known to negatively regulate stomatal density. In some embodiments, stomatal conductance is increased by transgenic downregulation of Epidermal Patterning Factors Like proteins (e.g., EPFL1 and/or EPFL2).

In some embodiments, stomatal flux is modified through the transgenic mediated upregulation of MYB-like transcription factors associated with positive regulation of stomatal density. In some embodiments, stomatal conductance is modified through the transgenic expression of a GT2 like gene. In some embodiments, stomatal conductance is increased through the transgenic overexpression of a GT2 like gene.

In some embodiments, compositions and methods described herein comprise a combination of both negative stomatal density regulatory gene downregulation and positive stomatal density regulatory gene upregulation. In some embodiments, these combinations provide increased stomatal density leading to an increased gas exchange rate.

### Epidermal Patterning Factor-Like Protein 9 (EPF9)

In some embodiments, compositions and methods described herein comprise a transgenic Epidermal Patterning Factor-Like protein 9 (EPFL9) gene (also known as Stomagen). In some embodiments, EPFL9 genes produce an EPFL9 protein. In some embodiments, EPFL9 proteins are cleaved and secreted as a peptide. In some embodiments, EPFL9 functions to promote stomatal development. In some embodiments, EPFL9 is upregulated through transgene introduction. In some embodiments, an EPFL9 gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 99 or 101 (or a portion thereof). In some embodiments, an EPFL9 gene and/or transgene comprises a nucleotide sequence that is at least 90%, 91%, 92%, 93%, 94%, 95%, 96%, 97%, 98%, 99%, or 100% identical to any one of SEQ ID NOs: 98 or 100 (or a portion thereof).

-Exemplary Arabidopsis thaliana Epidermal Patterning Factor-Like
protein 9 (AtStomagen)Nucleic Acid Coding Sequence
SEQ ID NO: 98
ATGAAACATGAAATGATGAACATTAAACCAAGATGCATTACAATATTTTTCTTATTGTTCGCTC
TGTTACTGGGAAACTATGTCGTACAGGCCTCCAGGCCTAGGTCCATAGAGAACACAGTTTCTCT
GTTGCCACAAGTCCACCTTTTAAATTCGCGAAGGAGACACATGATCGGGAGCACTGCACCAACA
TGTACTTATAATGAATGTAGAGGTTGTCGTTACAAATGTAGGGCAGAACAGGTGCCTGTAGAAG
GGAACGATCCTATTAACAGTGCATATCATTACCGCTGCGTGTGTCACAGGTGA
-Exemplary Arabidopsis thaliana Epidermal Patterning Factor-Like
protein 9 (AtStomagen) Amino Acid Sequence
SEQ ID NO: 99
MKHEMMNIKPRCITIFFLLFALLLGNYVVQASRPRSIENTVSLLPQVHLLNSRRRHMIGSTAPT
CTYNECRGCRYKCRAEQVPVEGNDPINSAYHYRCVCHR
-Exemplary Oryza sativa Epidermal Patterning Factor-Like protein 9, X1
and/or X2 (OsStomagenX1 and/or X2) Amino Acid Sequence
SEQ ID NO: 100
MANACPTSTTSSLPLFFLFCFLLFSHARCNQGHHGSISGTDYGEQYPHQTLPEEHIHLQENIKV
LNKERLPKYARRMLIGSTAPICTYNECRGCRFKCTAEQVPVDANDPMNSAYHYKCVCHR
-Exemplary Epipremnum aureum Epidermal Patterning Factor-Like
protein 9 (EaStomagen) Amino Acid Sequence
SEQ ID NO: 101
MIGSTAPTCSYNECRGCRFRCRAEQVPVDANDPINSAYHYRCVCHR

### Caprice (CPC)

In some embodiments, compositions and methods described herein comprise a transgenic Caprice gene. In some embodiments, a Caprice gene produces an R3-type MYB transcription factor protein. In some embodiments, R3-type MYB transcription factor proteins act to mediate transcription of pro-stomatal formation genes. In some embodiments, R3-type MYB transcription factors (e.g., as encoded by Caprice) function to promote stomatal development. In some embodiments, Caprice is upregulated through transgene introduction. In some embodiments, a Caprice gene and/or transgene comprises a sequence encoding a peptide sequence that is at least 90%, 91%, 92%, 93%, 94%, 95%, 96%, 97%, 98%, 99%, or 100% identical to SEQ ID NO: 103 (or a portion thereof). In some embodiments, a Caprice gene and/or om transgene comprises a nucleotide sequence that is at least 90%, 91%, 92%, 93%, 94%, 95%, 96%, 97%, 98%, 99%, or 100% identical to SEQ ID NO:102 (or a portion thereof).

-Exemplary Arabidopsis thaliana R3-type MYB transcription factor
(AtCaprice) Nucleotide Coding Sequence
SEQ ID NO: 102
ATGTTTAGAAGCGACAAGGCCGAGAAGATGGACAAACGACGGCGCAGGCAATCAAAAGCTAAGG
CATCCTGTTCTGAGGAAGTAAGTTCAATAGAATGGGAAGCTGTGAAAATGAGCGAAGAGGAAGA
GGATTTGATATCAAGAATGTATAAACTCGTGGGTGACAGATGGGAGTTAATAGCCGGGAGAATT
CCTGGTAGGACACCTGAAGAGATCGAGAGATATTGGTTGATGAAACATGGAGTAGTTTTCGCAA
ATCGGAGGCGAGACTTTTTCAGAAAGTGA
-Exemplary Arabidopsis thaliana R3-type MYB transcription factor
(AtCaprice) Amino Acid Sequence
SEQ ID NO: 103
MFRSDKAEKMDKRRRRQSKAKASCSEEVSSIEWEAVKMSEEEEDLISRMYKLVGDRWELIAGRI
PGRTPEEIERYWLMKHGVVFANRRRDFFRK

### MYB-Like Transcription Factor GT-2

In some embodiments, compositions and methods described herein comprise a transgenic GT-2 like gene. In some embodiments, a GT-2 like gene produces a MYB-like transcription factor protein. In some embodiments, a MYB-like transcription factor protein acts to mediate transcription of pro-stomatal formation genes. In some embodiments, a MYB-like transcription factor (e.g., as encoded by GT-2 like genes) functions to promote stomatal development. In some embodiments, GT-2 like genes are upregulated through transgene introduction. In some embodiments, a GT-2 like gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 105, 107, or 109 (or a portion thereof). In some embodiments, a GT-2 like gene and/or transgene comprises a nucleotide sequence that is at least 90%, 91%, 92%, 93%, 94%, 95%, 96%, 97%, 98%, 99%, or 100% identical to any one of SEQ ID NOs: 104, 106, or 108 (or a portion thereof).

-Exemplary Arabidopsis thaliana MYB-like transcription factor (GT-2
like 1.1) Nucleotide Coding Sequence
SEQ ID NO: 104
ATGGAGCAAGGAGGAGGTGGTGGTGGTAATGAAGTTGTGGAGGAAGCTTCACCTATTAGTTCAA
GACCTCCTGCTAACAACTTAGAAGAGCTTATGAGATTCTCAGCCGCCGCGGATGACGGTGGATT
AGGAGGTGGAGGTGGAGGAGGAGGAGGAGGAAGTGCTTCTTCTTCATCGGGAAATCGATGGCCG
AGAGAAGAAACTTTAGCTCTTCTTCGGATCCGATCCGATATGGATTCTACTTTTCGTGATGCTA
CTCTCAAAGCTCCTCTTTGGGAACATGTTTCCAGGAAGCTATTGGAGTTAGGTTACAAACGAAG
TTCAAAGAAATGCAAAGAGAAATTCGAAAACGTTCAGAAATATTACAAACGTACTAAAGAAACT
CGCGGTGGTCGTCATGATGGTAAAGCTTACAAGTTCTTCTCTCAGCTTGAAGCTCTCAACACTA
CTCCTCCTTCATCTTCCCTCGACGTTACTCCTCTCTCCGTCGCTAATCCCATTCTCATGCCTTC
TTCTTCTTCTTCTCCATTTCCCGTATTCTCTCAACCGCAACCGCAAACGCAAACGCAACCGCCT
CAAACGCATAATGTCTCTTTTACTCCTACTCCACCACCTCTTCCACTTCCTTCAATGGGTCCGA
TATTTACCGGTGTTACTTTCTCGTCTCATAGCTCATCGACGGCTTCAGGAATGGGGTCTGATGA
TGATGACGACGATATGGACGTTGATCAGGCTAACATTGCGGGTTCTAGTAGCCGAAAACGCAAA
CGTGGAAACCGCGGTGGAGGCGGTAAAATGATGGAATTGTTTGAAGGTTTGGTGAGACAAGTAA
TGCAAAAGCAAGCGGCTATGCAAAGGAGTTTCTTGGAAGCTCTTGAGAAGAGAGAGCAAGAACG
TCTTGATCGTGAAGAAGCTTGGAAACGTCAAGAAATGGCTCGGTTAGCTCGAGAACACGAGGTC
ATGTCTCAAGAACGAGCCGCCTCTGCTTCTCGTGACGCCGCAATCATTTCATTGATTCAGAAAA
TTACTGGCCATACCATTCAGTTACCTCCTTCTTTGTCATCTCAACCGCCTCCACCGTATCAACC
GCCACCCGCGGTCACTAAACGTGTGGCGGAACCACCATTATCAACAGCTCAATCTCAATCACAA
CAACCAATAATGGCGATTCCACAACAACAAATTCTTCCTCCTCCTCCTCCTTCTCATCCTCACG
CTCATCAACCAGAACAGAAACAACAACAACAACCACAACAAGAGATGGTCATGAGCTCGGAACA
ATCATCATTACCATCATCATCAAGATGGCCAAAGGCAGAGATTCTAGCGCTTATAAACCTGAGA
AGTGGAATGGAACCAAGGTACCAAGATAATGTACCTAAAGGACTTCTATGGGAAGAGATCTCAA
CTTCAATGAAGAGAATGGGATACAACAGAAACGCTAAGAGATGTAAAGAGAAATGGGAAAACAT
AAACAAATACTACAAGAAAGTTAAAGAAAGCAACAAGAAACGTCCTCAAGATGCTAAGACTTGT
CCTTACTTTCACCGCCTCGATCTTCTTTACCGCAACAAAGTACTCGGTAGTGGCGGTGGTTCTA
GCACTTCTGGTCTACCTCAAGACCAAAAACAGAGTCCGGTCACTGCGATGAAACCGCCACAAGA
AGGACTTGTTAATGTTCAACAAACTCATGGGTCAGCTTCAACTGAGGAAGAAGAGCCTATAGAG
GAAAGTCCACAAGGAACAGAAAAGCCAGAAGACCTTGTGATGAGAGAGCTGATTCAACAACAAC
AGCAACTACAACAACAAGAATCAATGATAGGTGAGTATGAAAAGATTGAAGAGTCTCACAATTA
TAATAACATGGAGGAAGAGGAAGATCAGGAAATGGATGAGGAAGAACTAGACGAGGATGAGAAG
TCCGCGGCTTTCGAGATTGCGTTTCAAAGCCCTGCAAACAGAGGAGGCAATGGCCATACGGAAC
CACCTTTCTTGACAATGGTTCAGTAA
-Exemplary Arabidopsis thaliana MYB-like transcription factor (GT-2
like 1.1) Amino Acid Sequence
SEQ ID NO: 105
MEQGGGGGGNEVVEEASPISSRPPANNLEELMRFSAAADDGGLGGGGGGGGGGSASSSSGNRWP
REETLALLRIRSDMDSTFRDATLKAPLWEHVSRKLLELGYKRSSKKCKEKFENVQKYYKRTKET
RGGRHDGKAYKFFSQLEALNTTPPSSSLDVTPLSVANPILMPSSSSSPFPVFSQPQPQTQTQPP
QTHNVSFTPTPPPLPLPSMGPIFTGVTFSSHSSSTASGMGSDDDDDDMDVDQANIAGSSSRKRK
RGNRGGGGKMMELFEGLVRQVMQKQAAMQRSFLEALEKREQERLDREEAWKRQEMARLAREHEV
MSQERAASASRDAAIISLIQKITGHTIQLPPSLSSQPPPPYQPPPAVTKRVAEPPLSTAQSQSQ
QPIMAIPQQQILPPPPPSHPHAHQPEQKQQQQPQQEMVMSSEQSSLPSSSRWPKAEILALINLR
SGMEPRYQDNVPKGLLWEEISTSMKRMGYNRNAKRCKEKWENINKYYKKVKESNKKRPQDAKTC
PYFHRLDLLYRNKVLGSGGGSSTSGLPQDQKQSPVTAMKPPQEGLVNVQQTHGSASTEEEEPIE
ESPQGTEKPEDLVMRELIQQQQQLQQQESMIGEYEKIEESHNYNNMEEEEDQEMDEEELDEDEK
SAAFEIAFQSPANRGGNGHTEPPFLIMVQ
-Exemplary Arabidopsis thaliana MYB-like transcription factor (GT-2
like 1.2) Nucleotide Coding Sequence
SEQ ID NO: 106
ATGAGTTTCTGGGACGTTTTCGATTTTGAAAATCCCAAGACTCTCTTTACTTCCAAAAAAAAAA
AAAAAAAATCCGATCGAACAGTAACCATAAAAATTTTCCAGCTAATAACGACAACCAAAAATAA
AATAAAACTAGAGAATCTGAATTATTTTCATGTTTTTGGAAACAGGAAGCTATTGGAGTTAGGT
TACAAACGAAGTTCAAAGAAATGCAAAGAGAAATTCGAAAACGTTCAGAAATATTACAAACGTA
CTAAAGAAACTCGCGGTGGTCGTCATGATGGTAAAGCTTACAAGTTCTTCTCTCAGCTTGAAGC
TCTCAACACTACTCCTCCTTCATCTTCCCTCGACGTTACTCCTCTCTCCGTCGCTAATCCCATT
CTCATGCCTTCTTCTTCTTCTTCTCCATTTCCCGTATTCTCTCAACCGCAACCGCAAACGCAAA
CGCAACCGCCTCAAACGCATAATGTCTCTTTTACTCCTACTCCACCACCTCTTCCACTTCCTTC
AATGGGTCCGATATTTACCGGTGTTACTTTCTCGTCTCATAGCTCATCGACGGCTTCAGGAATG
GGGTCTGATGATGATGACGACGATATGGACGTTGATCAGGCTAACATTGCGGGTTCTAGTAGCC
GAAAACGCAAACGTGGAAACCGCGGTGGAGGCGGTAAAATGATGGAATTGTTTGAAGGTTTGGT
GAGACAAGTAATGCAAAAGCAAGCGGCTATGCAAAGGAGTTTCTTGGAAGCTCTTGAGAAGAGA
GAGCAAGAACGTCTTGATCGTGAAGAAGCTTGGAAACGTCAAGAAATGGCTCGGTTAGCTCGAG
AACACGAGGTCATGTCTCAAGAACGAGCCGCCTCTGCTTCTCGTGACGCCGCAATCATTTCATT
GATTCAGAAAATTACTGGCCATACCATTCAGTTACCTCCTTCTTTGTCATCTCAACCGCCTCCA
CCGTATCAACCGCCACCCGCGGTCACTAAACGTGTGGCGGAACCACCATTATCAACAGCTCAAT
CTCAATCACAACAACCAATAATGGCGATTCCACAACAACAAATTCTTCCTCCTCCTCCTCCTTC
TCATCCTCACGCTCATCAACCAGAACAGAAACAACAACAACAACCACAACAAGAGATGGTCATG
AGCTCGGAACAATCATCATTACCATCATCATCAAGATGGCCAAAGGCAGAGATTCTAGCGCTTA
TAAACCTGAGAAGTGGAATGGAACCAAGGTACCAAGATAATGTACCTAAAGGACTTCTATGGGA
AGAGATCTCAACTTCAATGAAGAGAATGGGATACAACAGAAACGCTAAGAGATGTAAAGAGAAA
TGGGAAAACATAAACAAATACTACAAGAAAGTTAAAGAAAGCAACAAGAAACGTCCTCAAGATG
CTAAGACTTGTCCTTACTTTCACCGCCTCGATCTTCTTTACCGCAACAAAGTACTCGGTAGTGG
CGGTGGTTCTAGCACTTCTGGTCTACCTCAAGACCAAAAACAGAGTCCGGTCACTGCGATGAAA
CCGCCACAAGAAGGACTTGTTAATGTTCAACAAACTCATGGGTCAGCTTCAACTGAGGAAGAAG
AGCCTATAGAGGAAAGTCCACAAGGAACAGAAAAGCCAGAAGACCTTGTGATGAGAGAGCTGAT
TCAACAACAACAGCAACTACAACAACAAGAATCAATGATAGGTGAGTATGAAAAGATTGAAGAG
TCTCACAATTATAATAACATGGAGGAAGAGGAAGATCAGGAAATGGATGAGGAAGAACTAGACG
AGGATGAGAAGTCCGCGGCTTTCGAGATTGCGTTTCAAAGCCCTGCAAACAGAGGAGGCAATGG
CCATACGGAACCACCTTTCTTGACAATGGTTCAGTAA
-Exemplary Arabidopsis thaliana MYB-like transcription factor (GT-2
like 1.2) Amino Acid Sequence
SEQ ID NO: 107
MSFWDVFDFENPKTLFTSKKKKKKSDRTVTIKIFQLITTTKNKIKLENLNYFHVFGNRKLLELG
YKRSSKKCKEKFENVQKYYKRTKETRGGRHDGKAYKFFSQLEALNTTPPSSSLDVTPLSVANPI
LMPSSSSSPFPVFSQPQPQTQTQPPQTHNVSFTPTPPPLPLPSMGPIFTGVTFSSHSSSTASGM
GSDDDDDDMDVDQANIAGSSSRKRKRGNRGGGGKMMELFEGLVRQVMQKQAAMQRSFLEALEKR
EQERLDREEAWKRQEMARLAREHEVMSQERAASASRDAAIISLIQKITGHTIQLPPSLSSQPPP
PYQPPPAVTKRVAEPPLSTAQSQSQQPIMAIPQQQILPPPPPSHPHAHQPEQKQQQQPQQEMVM
SSEQSSLPSSSRWPKAEILALINLRSGMEPRYQDNVPKGLLWEEISTSMKRMGYNRNAKRCKEK
WENINKYYKKVKESNKKRPQDAKTCPYFHRLDLLYRNKVLGSGGGSSTSGLPQDQKQSPVTAMK
PPQEGLVNVQQTHGSASTEEEEPIEESPQGTEKPEDLVMRELIQQQQQLQQQESMIGEYEKIEE
SHNYNNMEEEEDQEMDEEELDEDEKSAAFEIAFQSPANRGGNGHTEPPFLTMVQ
-Exemplary Arabidopsis thaliana MYB-like transcription factor (GT-2
like 1.3) Nucleotide Coding Sequence
SEQ ID NO: 108
ATGGAGCAAGGAGGAGGTGGTGGTGGTAATGAAGTTGTGGAGGAAGCTTCACCTATTAGTTCAA
GACCTCCTGCTAACAACTTAGAAGAGCTTATGAGATTCTCAGCCGCCGCGGATGACGGTGGATT
AGGAGGTGGAGGTGGAGGAGGAGGAGGAGGAAGTGCTTCTTCTTCATCGGGAAATCGATGGCCG
AGAGAAGAAACTTTAGCTCTTCTTCGGATCCGATCCGATATGGATTCTACTTTTCGTGATGCTA
CTCTCAAAGCTCCTCTTTGGGAACATGTTTCCAGGAAGCTATTGGAGTTAGGTTACAAACGAAG
TTCAAAGAAATGCAAAGAGAAATTCGAAAACGTTCAGAAATATTACAAACGTACTAAAGAAACT
CGCGGTGGTCGTCATGATGGTAAAGCTTACAAGTTCTTCTCTCAGCTTGAAGCTCTCAACACTA
CTCCTCCTTCATCTTCCCTCGACGTTACTCCTCTCTCCGTCGCTAATCCCATTCTCATGCCTTC
TTCTTCTTCTTCTCCATTTCCCGTATTCTCTCAACCGCAACCGCAAACGCAAACGCAACCGCCT
CAAACGCATAATGTCTCTTTTACTCCTACTCCACCACCTCTTCCACTTCCTTCAATGGGTCCGA
TATTTACCGGTGTTACTTTCTCGTCTCATAGCTCATCGACGGCTTCAGGAATGGGGTCTGATGA
TGATGACGACGATATGGACGTTGATCAGGCTAACATTGCGGGTTCTAGTAGCCGAAAACGCAAA
CGTGGAAACCGCGGTGGAGGCGGTAAAATGATGGAATTGTTTGAAGGTTTGGTGAGACAAGTAA
TGCAAAAGCAAGCGGCTATGCAAAGGAGTTTCTTGGAAGCTCTTGAGAAGAGAGAGCAAGAACG
TCTTGATCGTGAAGAAGCTTGGAAACGTCAAGAAATGGCTCGGTTAGCTCGAGAACACGAGGTC
ATGTCTCAAGAACGAGCCGCCTCTGCTTCTCGTGACGCCGCAATCATTTCATTGATTCAGAAAA
TTACTGGCCATACCATTCAGTTACCTCCTTCTTTGTCATCTCAACCGCCTCCACCGTATCAACC
GCCACCCGCGGTCACTAAACGTGTGGCGGAACCACCATTATCAACAGCTCAATCTCAATCACAA
CAACCAATAATGGCGATTCCACAACAACAAATTCTTCCTCCTCCTCCTCCTTCTCATCCTCACG
CTCATCAACCAGAACAGAAACAACAACAACAACCACAACAAGAGATGGTCATGAGCTCGGAACA
ATCATCATTACCATCATCATCAAGATGGCCAAAGGCAGAGATTCTAGCGCTTATAAACCTGAGA
AGTGGAATGGAACCAAGGTACCAAGATAATGTACCTAAAGGACTTCTATGGGAAGAGATCTCAA
CTTCAATGAAGAGAATGGGATACAACAGAAACGCTAAGAGATGTAAAGAGAAATGGGAAAACAT
AAACAAATACTACAAGAAAGTTAAAGAAAGCAACAAGAAACGTCCTCAAGATGCTAAGACTTGT
CCTTACTTTCACCGCCTCGATCTTCTTTACCGCAACAAAGTACTCGGTAGTGGCGGTGGTTCTA
GCACTTCTGGTCTACCTCAAGACCAAAAACAGAGTCCGGTCACTGCGATGAAACCGCCACAAGA
AGGACTTGTTAATGTTCAACAAACTCATGGGTCAGCTTCAACTGAGGAAGAAGAGCCTATAGAG
GAAAGTCCACAAGGAACAGAAAAGGTACAAACTTTGCTTTTCCTTGTCAAAATGTGA
-Exemplary Arabidopsis thaliana MYB-like transcription factor (GT-2
like 1.3) Amino Acid Sequence
SEQ ID NO: 109
MEQGGGGGGNEVVEEASPISSRPPANNLEELMRFSAAADDGGLGGGGGGGGGGSASSSSGNRWP
REETLALLRIRSDMDSTFRDATLKAPLWEHVSRKLLELGYKRSSKKCKEKFENVQKYYKRTKET
RGGRHDGKAYKFFSQLEALNTTPPSSSLDVTPLSVANPILMPSSSSSPFPVFSQPQPQTQTQPP
QTHNVSFTPTPPPLPLPSMGPIFTGVTFSSHSSSTASGMGSDDDDDDMDVDQANIAGSSSRKRK
RGNRGGGGKMMELFEGLVRQVMQKQAAMQRSFLEALEKREQERLDREEAWKRQEMARLAREHEV
MSQERAASASRDAAIISLIQKITGHTIQLPPSLSSQPPPPYQPPPAVTKRVAEPPLSTAQSQSQ
QPIMAIPQQQILPPPPPSHPHAHQPEQKQQQQPQQEMVMSSEQSSLPSSSRWPKAEILALINLR
SGMEPRYQDNVPKGLLWEEISTSMKRMGYNRNAKRCKEKWENINKYYKKVKESNKKRPQDAKTC
PYFHRLDLLYRNKVLGSGGGSSTSGLPQDQKQSPVTAMKPPQEGLVNVQQTHGSASTEEEEPIE
ESPQGTEKVQTLLFLVKM

### Modifying Cuticle Wax Levels

In some embodiments, compositions and methods of the present disclosure comprise modified (e.g., increased) levels of certain plant cuticle waxes. In some embodiments, such a modification is facilitated through transgene introduction, gene knockdown, and/or gene knockout using materials and methods described herein.

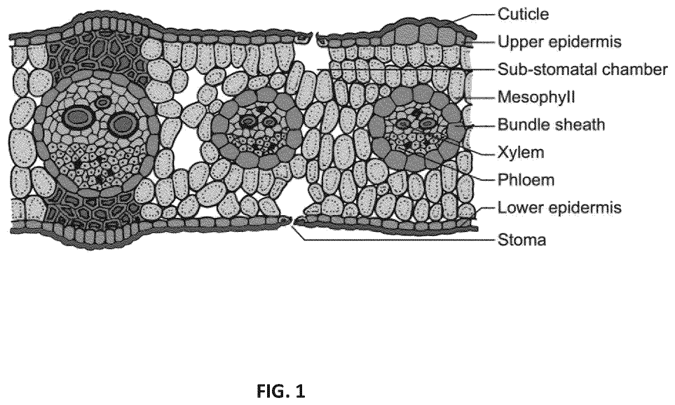

A plant cuticle is an extracellular lipophilic biopolymer that often covers both leaf and fruit surfaces (see FIG. 1). It is thought that the cuticle's main function is the protection of land-living plants from uncontrolled water loss. In the past, the permeability of the cuticle to water and to non-ionic lipophilic molecules (pesticides, herbicides and other xenobiotics) was studied intensively, whereas cuticular penetration of polar ionic compounds was rarely investigated.

In most cases, the plant cuticle membrane is composed of the depolymerizable biopolymer cutin (Kolattukudy, 2001), the non-depolymerizable polymer cutan (Tegelaar et al., 1993) and associated soluble cuticular lipids also called cuticular waxes (Jenks and Ashworth, 2003). In general, waxes are predominantly linear, long-chain, aliphatic molecules with different functionalities (alkanes, alcohols, aldehydes, acids, etc.). In general, waxes are solid, partially crystalline aggregates at room temperature (Reynhardt, 1997). In some embodiments, waxes can be found in the outer parts of the cutin polymer (intra-cuticular waxes) and on its surface (epicuticular waxes). In some embodiments, the permeability of the cuticle to water and to organic compounds increases upon wax extraction by factors between 10 and 1000, in such cases, it may be concluded that the cuticular transport barrier is largely formed by these cuticular waxes (Schönherr, 1976).

In some embodiments, a phyllosphere and/or endosphere (e.g., the above-ground parts of the plant) represent a major battleground for plant-microbe interactions (Junker and Tholl, 2013). In some embodiments, these surfaces are covered by a matrix collectively designated as (epi) cuticular waxes (Buschhaus and Jetter, 2011): complex mixtures of hydrophobic compounds such as long-chain esters-compounds chemically considered as waxes (Bruice, 2006)—and other lipophilic compounds such as saturated aliphatic hydrocarbon chains of at least 20 carbons, pentacyclic triterpenoids, and phenylpropanoids (Vogg et al., 2004; Kunst and Samuels, 2009; Buschhaus and Jetter, 2011; Hama et al., 2019). Thus, due to the lipophilic nature of these epicuticular waxes, it has been proposed that endogenous VOCs can accumulate in the epicuticular wax layers of plants (Widhalm et al., 2015).

In some embodiments, VOCs can also be sequestered by plant cuticular waxes. In such an embodiment, certain VOCs may maintain their biological activity, and such a sequestered VOCs could generate a “passive” associational resistance and/or selective pressure that is independent of a gene expression in a host plant.

In some embodiments, a pathway for VOC uptake by an aboveground portion of a plant parts is likely dependent on properties of a VOCs. In some embodiments, a hydrophilic VOC such as formaldehyde may not diffuse easily through the cuticle that consists of lipids, whereas, in some embodiments, a lipophilic VOC such as benzene is more likely to penetrate through such a cuticle. In some embodiments, relative importance of stomatal uptake compared to cuticular uptake may therefore be dependent on a VOC in question.

### Aldehyde Decarbonylase (CER1)

In some embodiments, long-chain alkanes are synthesized from fatty acids through the intermediacy of the corresponding fatty aldehydes. Such molecules act as substrates for a group of enzymes, the aldehyde decarbonylases, which catalyze the removal of the aldehyde carbonyl group to form the alkane. It is predicted that such enzymes are likely to be integral membrane proteins and contain an “eight histidine” motif common to stearoyl desaturases and fatty acid hydroxylases.

In some embodiments, an Aldehyde Decarbonylase gene and/or transgene comprises a sequence encoding a peptide sequence that is at least 90%, 91%, 92%, 93%, 94%, 95%, 96%, 97%, 98%, 99%, or 100% identical to SEQ ID NO: 111 (or a portion thereof). In some embodiments, an Aldehyde Decarbonylase gene and/or transgene comprises a nucleotide sequence that is at least 90%, 91%, 92%, 93%, 94%, 95%, 96%, 97%, 98%, 99%, or 100% identical to SEQ ID NO: 110 (or a portion thereof).

-Exemplary Nicotiana tabacum Aldehyde Decarbonylase (CER1, aka
Eceriferum 1) Nucleic Acid Coding Sequence
SEQ ID NO: 110
ATGGCTTCTAAACCAGGCATTCTAACAGAATGGCCATGGACATGGCTTGGGAACTTCAAGTACG
TGGTTTTGGCACCATATGTGGCTCACAGCCTACACTCATTCTTCATGAGCGAAGATGAAAGCAA
GAGGGATATCACATACTTAATTATATTTCCATTTCTACTCTTCCGAATGCTTCACAACCAGATA
TGGATATCCTTATCTCGCTACAGAACTGCCAAGGGTGATAACCGAATTGTTGACAAGAGCATTG
AATTTGATCAAGTTGACAGAGAAAGAAACTGGGATGATCAGATCATACTTAACGGACTGCTGTT
CTACTATGGATACACGAAGCTGGAGCAGTCTCATCACATGCCTATTTGGAGGACAGATGGGATC
ATTATGACAGCTTTGCTCCAAACTGGTCCTGTTGAATTTCTCTACTATTGGCTTCACAGAGCTT
TACACCACCATTTCCTTTACTCTCGCTATCATTCTCATCACCATTCCTCCATTGTCACTGAACC
CATTACTTCTGTGATTCATCCATTTGCAGAGCATATAGCATACTTCTTGCTATTTGCCATCCCA
CTTCTCACAACTGTGCTAACTGGGACTGCTTCAATAGTTTCATTTGGTGGATATATTACTTATA
TTGATTTTATGAATAACATGGGGCATTGCAACTTTGAGATCATTCCAAAGTGGATGTTCTCCAG
CTTTCCCCCTCTCAAATACTTGATGTATACACCCTCGTATCATTCACTCCATCACACTCAATTT
AGAACAAACTACTCGCTTTTTATGCCAATGTACGATTACATTTACGATACACTAGACAAATCTT
CAGACACATTATACGAAAAATCACTTGAAAGGCAAGGCAAATCGCCGGATGTGGTGCACCTAAC
ACACCTAACAACCCCAGAATCCATTTACCATCTCAGGCTAGGATTTGCTTCTTTTGCCTCGGAA
CCTTACACCTCTAAGTGGTATTTTTGGTTAATGTGGCCTGTTACATTGTGGTCTATGATGATTA
CTTGGATTTATGGTCACACATTTACTGTTGAGAGAAATGTGTTCAAGAGTCTGAATTTGCAAAC
TTGGGCGATCCCAAAATATCGCATACAATATTTTATGCAATGGCAAAGAGAGACGATTAACAAC
TTTATTGAGGAAGCTATCATGGAAGCAGATCGAAAAGGCATAAAAGTATTGAGCCTTGGACTCT
TAAATCAGGAGGAGCAACTGAATAATAATGGTGAGCTTTACATAAGAAGGCATCCTCAGCTCAA
AGTGAAGGTGGTTGATGGAAGTAGCCTAGCTGTTGCTGTGGTCCTAAACTCTATTCCTAAAGGA
ACCACACAAGTGGTCCTTGGAGGCCATTTGTCGAAAGTTGCAAATGCGATTGCCCTTGCCTTAT
GCCAAGGAGGAGTAAAGGTTGTGACATTGCGAGAAGAAGAGTACAAGAAGCTCAAATCAAGTCT
TACCCCTGAAGTCGCAATTAATTTGGTTCCCTCAAAAACATATGCTTCAAAGATATGGCTAGTA
GGGGATGGATTGAGTGAAGATGAACAATTGAAAGCACCAAAAGGAACATTATTCATTCCCTTTT
CACAATTCCCACCAAGGAAAGCTCGCAAGGATTGCCTCTACTTTCACACACCAGCCATGATCAC
TCCAAAACACTTTGAAAACGTGGACTCCTGTGAGAATTGGCTTCCAAGAAGAGTGATGAGCGCG
TGGCGAGTAGCTGGAATATTGCACGCACTGAAAGGCTGGAATGAGCATGAGTGTGGGAACATGA
TCTTTGATATTGAGAAAGTCTGGAAAGCAAGTCTTGATCACGGTTTTAGCCCATTGACTATGGC
TTCTGCTTCTGAATCCAAGGCTTAA
-Exemplary Nicotiana tabacum Aldehyde Decarbonylase (CER1, aka
Eceriferum 1) Amino Acid Sequence
SEQ ID NO: 111
MASKPGILTEWPWTWLGNFKYVVLAPYVAHSLHSFFMSEDESKRDITYLIIFPFLLFRMLHNQI
WISLSRYRTAKGDNRIVDKSIEFDQVDRERNWDDQIILNGLLFYYGYTKLEQSHHMPIWRIDGI
IMTALLQTGPVEFLYYWLHRALHHHFLYSRYHSHHHSSIVTEPITSVIHPFAEHIAYFLLFAIP
LLTTVLTGTASIVSFGGYITYIDFMNNMGHCNFEIIPKWMFSSFPPLKYLMYTPSYHSLHHTQF
RTNYSLFMPMYDYIYDTLDKSSDTLYEKSLERQGKSPDVVHLTHLTTPESIYHLRLGFASFASE
PYTSKWYFWLMWPVTLWSMMITWIYGHTFTVERNVFKSLNLQTWAIPKYRIQYFMQWQRETINN
FIEEAIMEADRKGIKVLSLGLLNQEEQLNNNGELYIRRHPQLKVKVVDGSSLAVAVVLNSIPKG
TTQVVLGGHLSKVANAIALALCQGGVKVVTLREEEYKKLKSSLTPEVAINLVPSKTYASKIWLV
GDGLSEDEQLKAPKGTLFIPFSQFPPRKARKDCLYFHTPAMITPKHFENVDSCENWLPRRVMSA
WRVAGILHALKGWNEHECGNMIFDIEKVWKASLDHGFSPLTMASASESKA

### 3-Ketoacyl-CoA-Synthase (CER6)

In some embodiments, a composition described herein comprises a transgenic 3-ketoacyl-CoA-synthase. Such an enzyme, among other things, contributes to cuticular wax and suberin biosynthesis and is involved in both decarbonylation and acyl-reduction wax synthesis pathways.

In some embodiments, a 3-ketoacyl-CoA-synthase gene and/or transgene comprises a sequence encoding a peptide sequence that is at least 90%, 91%, 92%, 93%, 94%, 95%, 96%, 97%, 98%, 99%, or 100% identical to SEQ ID NO: 113 (or a portion thereof). In some embodiments, a 3-ketoacyl-CoA-synthase gene and/or transgene comprises a nucleotide sequence that is at least 90%, 91%, 92%, 93%, 94%, 95%, 96%, 97%, 98%, 99%, or 100% identical to SEQ ID NO: 112 (or a portion thereof).

-Exemplary Nicotiana tabacum 3-ketoacyl-CoA-synthase (CER6, aka
Eceriferum 6) Nucleic Acid Coding Sequence
SEQ ID NO: 112
ATGGCAGAAGTAGTCCCAAGTTTCTCTAATTCAGTGAAGCTCAAATATGTCAAACTTGGTTATC
AATACCTTGTTAATCATATTCTAACATTTTTGCTTGTGCCTATTATGGTTGGTGTTACTATAGA
GGTATTAAGACTTGGCCCTGAAGAATTGCTAAGCATATGGAATTCACTCCACTTTGATCTTCTT
CAAATCCTTTGCTCTTCTTTTCCCATCATCTTCATAGCCACTGTTTACTTCATGTCCAAACCTC
GATCAATTTACCTTGTAGATTATTCATGTTACAAAGCTCCGGTTACCTGCCGAGTCCCATTTTC
AACTTTCATGGAACACTCTAGGCTCATTTTGAAGGATAATCCCAAGAGTGTCGAGTTCCAAATG
CGTATTCTTGAAAGGTCTGGCCTTGGAGAAGAAACGTGCTTGCCTCCTGCTATTCATTATATCC
CTCCAACACCAACTATGGAAGCTGCTAGAGGTGAAGCAGAAGTGGTCATATTCTCAGCAATTGA
TGACCTAATGAAGAAAACAGGACTCAAGCCAAAGGATATTGACATTCTTATTGTCAACTGCAGC
TTGTTTTCTCCAACTCCATCTTTATCAGCTATGGTAGTGAACAAATACAAGTTGAGAAGTAACA
TAAAAAGTTACAATCTTTCTGGTATGGGATGTAGTGCTGGTTTAATATCAATTGATTTAGCTAG
GGATCTTCTTCAAGTCCATCCAAATTCAAATGCTTTAGTTGTAAGCACTGAGATTATCACACCT
AATTATTACAAAGGTTCAGAGAGAGCAATGCTTCTACCAAATTGTTTGTTCCGTATGGGTGGTG
CAGCCATACTCTTGTCCAACAAAAGGCGCGATAGATACAGAGCAAAGTACAGATTAATGCACGT
GGTCCGAACACATAAGGGTGCAGATGATAAGGCATTTAAATGTGTATTTGAACAAGAAGATCCA
CAAGGGAAAGTTGGTATTAATTTATCAAAAGACCTTATGGTTATAGCAGGAGAAGCTTTAAAAT
CCAACATTACTACAATTGGTCCTTTAGTTCTTCCAGCATCAGAGCAACTCCTTTTTCTCCTCAC
ACTTATTAGTCGGAAATTTTTTAATCCCAAGTTGAAACCTTATATTCCGGATTTTAAACAAGCG
TTTGAACATTTTTGTATTCATGCGGGTGGTCGGGCTGTTATTGATGAACTTCAAAAGAACCTAC
AATTGTCTGCTGAACATGTTGAGGCATCAAGAATGACATTGCATAGATTTGGTAACACTTCATC
TTCTTCACTATGGTATGAGATGAGTTATATTGAGGCTAAAGGTAGGATGAAGAAAGGTGATAGA
GTTTGGCAGATTGCATTTGGGAGTGGATTTAAGTGTAACAGTGCTGTTTGGAAATGTAACAGAA
CAATAAAGACACCAACTGATGGGCCATGGCAAGATTGCATTGATAGGTATCCAGTCCACATTCC
AGAGATTGTCAAGCTCTAA
-Exemplary Nicotiana tabacum 3-ketoacyl-CoA-synthase (CER6, aka
Eceriferum 6) Amino Acid Sequence
SEQ ID NO: 113
MAEVVPSFSNSVKLKYVKLGYQYLVNHILTFLLVPIMVGVTIEVLRLGPEELLSIWNSLHFDLL
QILCSSFPIIFIATVYFMSKPRSTYLVDYSCYKAPVTCRVPFSTFMEHSRLILKDNPKSVEFQM
RILERSGLGEETCLPPAIHYIPPTPTMEAARGEAEVVIFSAIDDLMKKTGLKPKDIDILIVNCS
LFSPTPSLSAMVVNKYKLRSNIKSYNLSGMGCSAGLISIDLARDLLQVHPNSNALVVSTEIITP
NYYKGSERAMLLPNCLFRMGGAAILLSNKRRDRYRAKYRLMHVVRTHKGADDKAFKCVFEQEDP
QGKVGINLSKDLMVIAGEALKSNITTIGPLVLPASEQLLFLLTLISRKFFNPKLKPYIPDFKQA
FEHFCIHAGGRAVIDELQKNLQLSAEHVEASRMTLHRFGNTSSSSLWYEMSYIEAKGRMKKGDR
VWQIAFGSGFKCNSAVWKCNRTIKTPTDGPWQDCIDRYPVHIPEIVKL

### R2R3 MYB Transcription Factor

In some embodiments, a composition described herein comprises a transgenic R2R3 MYB transcription factor. Such a protein, among other things, may regulate different biological processes, such as primary and secondary metabolism, responses to biotic and abiotic stresses, developmental processes, and hormonal responses.

In some embodiments, a R2R3 MYB transcription factor gene and/or transgene comprises a sequence encoding a peptide sequence that is at least 90%, 91%, 92%, 93%, 94%, 95%, 96%, 97%, 98%, 99%, or 100% identical to SEQ ID NO: 115 (or a portion thereof). In some embodiments, a R2R3 MYB transcription factor gene and/or transgene comprises a nucleotide sequence that is at least 90%, 91%, 92%, 93%, 94%, 95%, 96%, 97%, 98%, 99%, or 100% identical to SEQ ID NO: 114 (or a portion thereof).

-Exemplary Nicotiana tabacum R2R3 MYB transcription factor (Myb-
related protein 306-like) Nucleic Acid Coding Sequence
SEQ ID NO: 114
ATGGGAAGGCCACCTTGTTGTGATAAAATAGGGGTGAAGAAAGGACCATGGACACCAGAAGAGG
ATATCATCTTGGTTTCATACATTCAACAACATGGTCCTGGTAACTGGAGAGCTGTTCCCAGTAA
TACTGGTTTGCTTAGATGCAGCAAAAGCTGTAGACTTAGATGGACTAATTATCTCCGTCCGGGA
ATCAAACGTGGCAACTTCACAGAACATGAAGAAAAGATGATTATTCACCTCCAAGCTCTTCTTG
GCAACAGATGGGCTGCGATAGCATCATATCTCCCACAAAGGACGGACAACGATATAAAAAATTA
CTGGAATACTCATCTGAGAAAGAAGCTGAAGAAACTTCAAGGGAATGATGAGAATAGTAATCAA
GAGGGAATACGCTCATCGTCTCAATCAAATGTCTCAAAAGGACAGTGGGAGAGGAGGCTTCAAA
CTGATATCCACATGGCTAAAAAAGCCCTTTGTGAGGCTTTGTCCCTTGACAAATCTGATTCTCC
GCCAAATAATCCTATCCCTCAACCTGTTCAATCATCTTGTACTTATGCATCTAGTGCTGAAAAT
ATTTCTCGATTGCTTCAAAATTGGATGAAAAATTCCCCCAAATCATCTCAATTTAGTCAATCAA
ACTCGGAGTGTACTACTCAAAGCTCCTTTAACAATTTATCAATCGGGCAGGGTTCGAGTTCTAG
TCCTAGTGAAGGGACCATAAGTGCAACAACACCCGAGGGTTTTGATCCGCTCTTTAGCTTCAAT
TCATCCAATACTGATATGTTGGCAGATGAGAGTAACGCTTTCACACCTGAAAATGCTAGGATTT
TTCAAGTTGAAAGCAAGCCAGATTTGCCGAATCTGAATGCTGAAAATGGATTTTTATTTCAAGA
GGAGAGCAAGCCAAGTTTGGAATCGGAAGTGCCATTAACTTTGCTGGAGAAGTGGCTCTTTGAT
GATGCTATTAATGCACCAGCACAAGAAAACCTAATGGGATTGGGAATAGGAATGGGAATGACCT
TGGGTGATGCTTCTGATTTGTTTTGA
-Exemplary Nicotiana tabacum R2R3 MYB transcription factor (Myb-
related protein 306-like) Amino Acid Sequence
SEQ ID NO: 115
MGRPPCCDKIGVKKGPWTPEEDIILVSYIQQHGPGNWRAVPSNTGLLRCSKSCRLRWTNYLRPG
IKRGNFTEHEEKMIIHLQALLGNRWAAIASYLPQRTDNDIKNYWNTHLRKKLKKLQGNDENSNQ
EGIRSSSQSNVSKGQWERRLQTDIHMAKKALCEALSLDKSDSPPNNPIPQPVQSSCTYASSAEN
ISRLLQNWMKNSPKSSQFSQSNSECTTQSSENNLSIGQGSSSSPSEGTISATTPEGFDPLFSFN
SSNTDMLADESNAFTPENARIFQVESKPDLPNLNAENGFLFQEESKPSLESEVPLILLEKWLFD
DAINAPAQENLMGLGIGMGMTLGDASDLF

### Wax Crystal-Sparse Leaf2/Glossy 1-1 (GL1-1)

In some embodiments, a composition described herein comprises a transgenic very-long chain aldehyde decarbonylase. In some embodiments, a very-long chain aldehyde decarbonylase is a homolog of CER3, WAX2, and/or GL1. In some embodiments, a very-long-chain aldehyde decarbonylase is GL1-1.

In some embodiments, a GL1-1 gene and/or transgene comprises a sequence encoding a peptide sequence that is at least 90%, 91%, 92%, 93%, 94%, 95%, 96%, 97%, 98%, 99%, or 100% identical to SEQ ID NO: 117 (or a portion thereof). In some embodiments, a GL1-1 gene and/or transgene comprises a nucleotide sequence that is at least 90%, 91%, 92%, 93%, 94%, 95%, 96%, 97%, 98%, 99%, or 100% identical to SEQ ID NO: 116 (or a portion thereof).

-Exemplary Oriza sativa very-long-chain aldehyde decarbonylase (GL1-1,
aka wax crystal-sparse leaf-2) Nucleotide Coding Sequence
SEQ ID NO: 116
ATGGGTGCCGCATTCTTGTCGTCGTGGCCATGGGATAACCTCGGCGCGTACAAGTATGTGTTGT
ACGCGCCGCTGGTGGGGAAGGCGGTGGCGGGGGGGGCGTGGGAGCGGGCGAGCCCCGACCACTG
GCTGCTGCTGCTGCTCGTCCTCTTCGGCGTCAGGGCCTTGACCTACCAGCTCTGGAGCTCGTTC
AGCAACATGCTCTTCGCCACCCGCCGCCGCCGCATCGTCCGCGACGGCGTCGACTTCGGCCAGA
TCGACAGGGAGTGGGACTGGGACAACTTCTTGATACTGCAGGTGCACATGGCGGCGGCGGCGTT
CTACGCGTTCCCGTCGCTGCGGCACCTCCCGCTGTGGGACGCCAGGGGCCTCGCCGTCGCCGCG
CTCCTCCACGTCGCCGCCACCGAGCCCCTGTTCTACGCCGCGCACAGGGCGTTCCACCGCGGCC
ACCTCTTCTCCTGCTACCACTTGCAACACCACTCCGCCAAGGTGCCCCAGCCATTCACAGCGGG
GTTCGCGACGCCGCTGGAGCAGCTGGTGCTGGGGGCGCTCATGGCGGTGCCGCTGGCGGCGGCG
TGCGCGGCGGGGCACGGCTCCGTCGCGCTGGCCTTCGCCTACGTGCTGGGTTTCGACAACCTCC
GCGCCATGGGCCACTGCAACGTCGAGGTGTTCCCCGGCGGCCTCTTCCAGTCGCTCCCCGTCCT
CAAATACCTTATCTACACCCCAACGTACCACACGATCCATCACACCAAGGAGGATGCCAACTTC
TGCCTGTTCATGCCGCTGTTCGACCTCATCGGTGGCACCCTCGACGCCCAGTCCTGGGAGATGC
AGAAGAAAACCAGCGCAGGGGTGGACGAGGTGCCGGAGTTCGTGTTCCTGGCGCACGTGGTGGA
CGTGATGCAGTCGCTGCACGTGCCGTTCGTGCTGCGGACGTTCGCGTCGACGCCCTTCTCGGTG
CAGCCGTTCCTGCTGCCCATGTGGCCGTTCGCGTTCCTCGTCATGCTCATGATGTGGGCGTGGT
CCAAGACCTTCGTCATCTCCTGCTACCGCCTCCGCGGCCGCCTCCACCAGATGTGGGCCGTCCC
CCGCTACGGCTTCCACTACTTCCTGCCGTTCGCCAAGGACGGCATCAACAACCAGATCGAGCTC
GCCATCCTCAGGGCGGACAAGATGGGCGCCAAGGTGGTCAGCCTCGCCGCTCTCAACAAGAATG
AGGCGCTGAACGGTGGCGGGACGCTGTTCGTGAACAAGCACCCGGGGCTCCGGGTGCGCGTCGT
CCACGGCAACACGCTGACGGCGGCGGTGATCCTCAACGAGATCCCGCAGGGCACCACCGAGGTG
TTCATGACCGGCGCCACGTCCAAGCTCGGCCGCGCCATCGCCCTCTACCTCTGCAGGAAGAAAG
TCCGCGTCATGATGATGACGCTGTCGACGGAGAGATTCCAGAAGATACAGAGGGAGGCGACGCC
GGAGCACCAGCAGTACCTGGTGCAGGTGACCAAGTACAGGTCGGCGCAGCACTGCAAGACGTGG
ATCGTCGGCAAGTGGCTGTCGCCGAGGGAGCAGCGTTGGGCGCCGCCGGGGACGCACTTCCACC
AGTTCGTCGTCCCCCCAATCATCGGCTTCCGCCGCGACTGCACCTACGGCAAGCTCGCCGCCAT
GCGCCTCCCCAAGGACGTCCAGGGCCTCGGCGCCTGCGAGTACTCGCTGGAGCGCGGGGTGGTG
CACGCGTGCCACGCCGGAGGCGTGGTGCACTTCCTGGAGGGGTACACGCACCACGAGGTGGGCG
CCATCGACGTGGACCGCATCGACGTCGTGTGGGAGGCGGCGCTCAGGCACGGCCTCCGGCCTGT
CTGA
-Exemplary Oriza sativa ver-long-chain aldehyde decarbonylase (GL1-1,
aka wax crystal-sparse leaf-2) Amino Acid Sequence
SEQ ID NO: 117
MGAAFLSSWPWDNLGAYKYVLYAPLVGKAVAGRAWERASPDHWLLLLLVLFGVRALTYQLWSSF
SNMLFATRRRRIVRDGVDFGQIDREWDWDNFLILQVHMAAAAFYAFPSLRHLPLWDARGLAVAA
LLHVAATEPLFYAAHRAFHRGHLFSCYHLQHHSAKVPQPFTAGFATPLEQLVLGALMAVPLAAA
CAAGHGSVALAFAYVLGFDNLRAMGHCNVEVFPGGLFQSLPVLKYLIYTPTYHTIHHTKEDANF
CLFMPLFDLIGGTLDAQSWEMQKKTSAGVDEVPEFVFLAHVVDVMQSLHVPFVLRTFASTPFSV
QPFLLPMWPFAFLVMLMMWAWSKIFVISCYRLRGRLHQMWAVPRYGFHYFLPFAKDGINNQIEL
AILRADKMGAKVVSLAALNKNEALNGGGTLFVNKHPGLRVRVVHGNTLTAAVILNEIPQGTTEV
FMTGATSKLGRAIALYLCRKKVRVMMMTLSTERFQKIQREATPEHQQYLVQVTKYRSAQHCKTW
IVGKWLSPREQRWAPPGTHFHQFVVPPIIGFRRDCTYGKLAAMRLPKDVQGLGACEYSLERGVV
HACHAGGVVHFLEGYTHHEVGAIDVDRIDVVWEAALRHGLRPV

### AP2/ERWEBP or AP2/ERF-Type Transcription Factor (Wrinkled)

In some embodiments, a composition described herein comprises a transgenic AP2/ERWEBP or AP2/ERF-type transcription factor. In some embodiments, a AP2/ERWEBP or AP2/ERF-type transcription factor is a WRINKLED protein.

In some embodiments, a AP2/ERWEBP or AP2/ERF-type transcription factor gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 119, 121, 123, 125, 127, 129, 131, or 133 (or a portion thereof). In some embodiments, a AP2/ERWEBP or AP2/ERF-type transcription factor gene and/or transgene comprises a nucleotide sequence that is at least 90%, 91%, 92%, 93%, 94%, 95%, 96%, 97%, 98%, 99%, or 100% identical to any one of SEQ ID NOs: 118, 120, 122, 124, 126, 128, 130, or 132 (or a portion thereof).

-Exemplary Arabidopsis thaliana AP2/ERWEBP TF (Wrinkled 1 isoform
1) Nucleotide Coding Sequence
SEQ ID NO: 118
ATGAAGAAGCGCTTAACCACTTCCACTTGTTCTTCTTCTCCATCTTCCTCTGTTTCTTCTTCTA
CTACTACTTCCTCTCCTATTCAGTCGGAGGCTCCAAGGCCTAAACGAGCCAAAAGGGCTAAGAA
ATCTTCTCCTTCTGGTGATAAATCTCATAACCCGACAAGCCCTGCTTCTACCCGACGCAGCTCT
ATCTACAGAGGAGTCACTAGACATAGATGGACTGGGAGATTCGAGGCTCATCTTTGGGACAAAA
GCTCTTGGAATTCGATTCAGAACAAGAAAGGCAAACAAGTTTATCTGGGAGCATATGACAGTGA
AGAAGCAGCAGCACATACGTACGATCTGGCTGCTCTCAAGTACTGGGGACCCGACACCATCTTG
AATTTTCCGGCAGAGACGTACACAAAGGAATTGGAAGAAATGCAGAGAGTGACAAAGGAAGAAT
ATTTGGCTTCTCTCCGCCGCCAGAGCAGTGGTTTCTCCAGAGGCGTCTCTAAATATCGCGGCGT
CGCTAGGCATCACCACAACGGAAGATGGGAGGCTCGGATCGGAAGAGTGTTTGGGAACAAGTAC
TTGTACCTCGGCACCTATAATACGCAGGAGGAAGCTGCTGCAGCATATGACATGGCTGCGATTG
AGTATCGAGGCGCAAACGCGGTTACTAATTTCGACATTAGTAATTACATTGACCGGTTAAAGAA
GAAAGGTGTTTTCCCGTTCCCTGTGAACCAAGCTAACCATCAAGAGGGTATTCTTGTTGAAGCC
AAACAAGAAGTTGAAACGAGAGAAGCGAAGGAAGAGCCTAGAGAAGAAGTGAAACAACAGTACG
TGGAAGAACCACCGCAAGAAGAAGAAGAGAAGGAAGAAGAGAAAGCAGAGCAACAAGAAGCAGA
GATTGTAGGATATTCAGAAGAAGCAGCAGTGGTCAATTGCTGCATAGACTCTTCAACCATAATG
GAAATGGATCGTTGTGGGGACAACAATGAGCTGGCTTGGAACTTCTGTATGATGGATACAGGGT
TTTCTCCGTTTTTGACTGATCAGAATCTCGCGAATGAGAATCCCATAGAGTATCCGGAGCTATT
CAATGAGTTAGCATTTGAGGACAACATCGACTTCATGTTCGATGATGGGAAGCACGAGTGCTTG
AACTTGGAAAATCTGGATTGTTGCGTGGTGGGAAGAGAGAGCCCACCCTCTTCTTCTTCACCAT
TGTCTTGCTTATCTACTGACTCTGCTTCATCAACAACAACAACAACAACCTCGGTTTCTTGTAA
CTATTTGTTTCAGGGCTTGTTCGTTGGTTCTGAATAA
-Exemplary Arabidopsis thaliana AP2/ERWEBP TF (Wrinkled 1 isoform
1) Amino Acid Sequence
SEQ ID NO: 119
MKKRLTTSTCSSSPSSSVSSSTTTSSPIQSEAPRPKRAKRAKKSSPSGDKSHNPTSPASTRRSS
IYRGVTRHRWTGRFEAHLWDKSSWNSIQNKKGKQVYLGAYDSEEAAAHTYDLAALKYWGPDTIL
NFPAETYTKELEEMQRVTKEEYLASLRRQSSGFSRGVSKYRGVARHHHNGRWEARIGRVFGNKY
LYLGTYNTQEEAAAAYDMAAIEYRGANAVTNFDISNYIDRLKKKGVFPFPVNQANHQEGILVEA
KQEVETREAKEEPREEVKQQYVEEPPQEEEEKEEEKAEQQEAEIVGYSEEAAVVNCCIDSSTIM
EMDRCGDNNELAWNFCMMDTGFSPFLTDQNLANENPIEYPELFNELAFEDNIDEMEDDGKHECL
NLENLDCCVVGRESPPSSSSPLSCLSTDSASSTTTTTTSVSCNYLFQGLFVGSE
-Exemplary Arabidopsis thaliana AP2/ERWEBP TF (Wrinkled 1 isoform
2) Nucleotide Coding Sequence
SEQ ID NO: 120
ATGCAGAGAGTGACAAAGGAAGAATATTTGGCTTCTCTCCGCCGCCAGAGCAGTGGTTTCTCCA
GAGGCGTCTCTAAATATCGCGGCGTCGCTAGGCATCACCACAACGGAAGATGGGAGGCTCGGAT
CGGAAGAGTGTTTGGGAACAAGTACTTGTACCTCGGCACCTATAATACGCAGGAGGAAGCTGCT
GCAGCATATGACATGGCTGCGATTGAGTATCGAGGCGCAAACGCGGTTACTAATTTCGACATTA
GTAATTACATTGACCGGTTAAAGAAGAAAGGTGTTTTCCCGTTCCCTGTGAACCAAGCTAACCA
TCAAGAGGGTATTCTTGTTGAAGCCAAACAAGAAGTTGAAACGAGAGAAGCGAAGGAAGAGCCT
AGAGAAGAAGTGAAACAACAGTACGTGGAAGAACCACCGCAAGAAGAAGAAGAGAAGGAAGAAG
AGAAAGCAGAGCAACAAGAAGCAGAGATTGTAGGATATTCAGAAGAAGCAGCAGTGGTCAATTG
CTGCATAGACTCTTCAACCATAATGGAAATGGATCGTTGTGGGGACAACAATGAGCTGGCTTGG
AACTTCTGTATGATGGATACAGGGTTTTCTCCGTTTTTGACTGATCAGAATCTCGCGAATGAGA
ATCCCATAGAGTATCCGGAGCTATTCAATGAGTTAGCATTTGAGGACAACATCGACTTCATGTT
CGATGATGGGAAGCACGAGTGCTTGAACTTGGAAAATCTGGATTGTTGCGTGGTGGGAAGAGAG
AGCCCACCCTCTTCTTCTTCACCATTGTCTTGCTTATCTACTGACTCTGCTTCATCAACAACAA
CAACAACAACCTCGGTTTCTTGTAACTATTTGGTCTGA
-Exemplary Arabidopsis thaliana AP2/ERWEBP TF (Wrinkled 1 isoform
2) Amino Acid Sequence
SEQ ID NO: 121
MQRVTKEEYLASLRRQSSGFSRGVSKYRGVARHHHNGRWEARIGRVFGNKYLYLGTYNTQEEAA
AAYDMAAIEYRGANAVTNFDISNYIDRLKKKGVFPFPVNQANHQEGILVEAKQEVETREAKEEP
REEVKQQYVEEPPQEEEEKEEEKAEQQEAEIVGYSEEAAVVNCCIDSSTIMEMDRCGDNNELAW
NFCMMDTGFSPFLTDQNLANENPIEYPELFNELAFEDNIDFMEDDGKHECLNLENLDCCVVGRE
SPPSSSSPLSCLSTDSASSTTTTTTSVSCNYLV
-Exemplary Arabidopsis thaliana AP2/ERWEBP TF (Wrinkled 1 isoform
3) Nucleotide Coding Sequence
SEQ ID NO: 122
ATGAAGAAGCGCTTAACCACTTCCACTTGTTCTTCTTCTCCATCTTCCTCTGTTTCTTCTTCTA
CTACTACTTCCTCTCCTATTCAGTCGGAGGCTCCAAGGCCTAAACGAGCCAAAAGGGCTAAGAA
ATCTTCTCCTTCTGGTGATAAATCTCATAACCCGACAAGCCCTGCTTCTACCCGACGCAGCTCT
ATCTACAGAGGAGTCACTAGACATAGATGGACTGGGAGATTCGAGGCTCATCTTTGGGACAAAA
GCTCTTGGAATTCGATTCAGAACAAGAAAGGCAAACAAGTTTATCTGGGAGCATATGACAGTGA
AGAAGCAGCAGCACATACGTACGATCTGGCTGCTCTCAAGTACTGGGGACCCGACACCATCTTG
AATTTTCCGGCAGAGACGTACACAAAGGAATTGGAAGAAATGCAGAGAGTGACAAAGGAAGAAT
ATTTGGCTTCTCTCCGCCGCCAGAGCAGTGGTTTCTCCAGAGGCGTCTCTAAATATCGCGGCGT
CGCTAGGCATCACCACAACGGAAGATGGGAGGCTCGGATCGGAAGAGTGTTTGGGAACAAGTAC
TTGTACCTCGGCACCTATAATACGCAGGAGGAAGCTGCTGCAGCATATGACATGGCTGCGATTG
AGTATCGAGGCGCAAACGCGGTTACTAATTTCGACATTAGTAATTACATTGACCGGTTAAAGAA
GAAAGGTGTTTTCCCGTTCCCTGTGAACCAAGCTAACCATCAAGAGGGTATTCTTGTTGAAGCC
AAACAAGAAGTTGAAACGAGAGAAGCGAAGGAAGAGCCTAGAGAAGAAGTGAAACAACAGTACG
TGGAAGAACCACCGCAAGAAGAAGAAGAGAAGGAAGAAGAGAAAGCAGAGCAACAAGAAGCAGA
GATTGTAGGATATTCAGAAGAAGCAGCAGTGGTCAATTGCTGCATAGACTCTTCAACCATAATG
GAAATGGATCGTTGTGGGGACAACAATGAGCTGGCTTGGAACTTCTGTATGATGGATACAGGGT
TTTCTCCGTTTTTGACTGATCAGAATCTCGCGAATGAGAATCCCATAGAGTATCCGGAGCTATT
CAATGAGTTAGCATTTGAGGACAACATCGACTTCATGTTCGATGATGGGAAGCACGAGTGCTTG
AACTTGGAAAATCTGGATTGTTGCGTGGTGGGAAGAGAGAGCCCACCCTCTTCTTCTTCACCAT
TGTCTTGCTTATCTACTGACTCTGCTTCATCAACAACAACAACAACAACCTCGGTTTCTTGTAA
CTATTTGGTCTGA
-Exemplary Arabidopsis thaliana AP2/ERWEBP TF (Wrinkled 1 isoform
3) Amino Acid Sequence
SEQ ID NO: 123
MKKRLTTSTCSSSPSSSVSSSTTTSSPIQSEAPRPKRAKRAKKSSPSGDKSHNPTSPASTRRSS
IYRGVTRHRWTGRFEAHLWDKSSWNSIQNKKGKQVYLGAYDSEEAAAHTYDLAALKYWGPDTIL
NFPAETYTKELEEMQRVTKEEYLASLRRQSSGFSRGVSKYRGVARHHHNGRWEARIGRVFGNKY
LYLGTYNTQEEAAAAYDMAAIEYRGANAVTNFDISNYIDRLKKKGVFPFPVNQANHQEGILVEA
KQEVETREAKEEPREEVKQQYVEEPPQEEEEKEEEKAEQQEAEIVGYSEEAAVVNCCIDSSTIM
EMDRCGDNNELAWNFCMMDTGFSPFLTDQNLANENPIEYPELFNELAFEDNIDFMEDDGKHECL
NLENLDCCVVGRESPPSSSSPLSCLSTDSASSTTTTTTSVSCNYLV
-Exemplary Arabidopsis thaliana AP2/ERWEBP TF (Wrinkled 1 isoform
4 and isoform 5) Nucleotide Coding Sequence
SEQ ID NO: 124
ATGATTTTGTTTGTTTTAATAAAGATCTGGACTTTAACTGATAAATTTGGTTTCTTTGATCTGT
TGTTTGATCTCAACTTCGTCACAACTTCACCAGTTTATCTGGGAGCATATGACAGTGAAGAAGC
AGCAGCACATACGTACGATCTGGCTGCTCTCAAGTACTGGGGACCCGACACCATCTTGAATTTT
CCGGCAGAGACGTACACAAAGGAATTGGAAGAAATGCAGAGAGTGACAAAGGAAGAATATTTGG
CTTCTCTCCGCCGCCAGAGCAGTGGTTTCTCCAGAGGCGTCTCTAAATATCGCGGCGTCGCTAG
GCATCACCACAACGGAAGATGGGAGGCTCGGATCGGAAGAGTGTTTGGGAACAAGTACTTGTAC
CTCGGCACCTATAATACGCAGGAGGAAGCTGCTGCAGCATATGACATGGCTGCGATTGAGTATC
GAGGCGCAAACGCGGTTACTAATTTCGACATTAGTAATTACATTGACCGGTTAAAGAAGAAAGG
TGTTTTCCCGTTCCCTGTGAACCAAGCTAACCATCAAGAGGGTATTCTTGTTGAAGCCAAACAA
GAAGTTGAAACGAGAGAAGCGAAGGAAGAGCCTAGAGAAGAAGTGAAACAACAGTACGTGGAAG
AACCACCGCAAGAAGAAGAAGAGAAGGAAGAAGAGAAAGCAGAGCAACAAGAAGCAGAGATTGT
AGGATATTCAGAAGAAGCAGCAGTGGTCAATTGCTGCATAGACTCTTCAACCATAATGGAAATG
GATCGTTGTGGGGACAACAATGAGCTGGCTTGGAACTTCTGTATGATGGATACAGGGTTTTCTC
CGTTTTTGACTGATCAGAATCTCGCGAATGAGAATCCCATAGAGTATCCGGAGCTATTCAATGA
GTTAGCATTTGAGGACAACATCGACTTCATGTTCGATGATGGGAAGCACGAGTGCTTGAACTTG
GAAAATCTGGATTGTTGCGTGGTGGGAAGAGAGAGCCCACCCTCTTCTTCTTCACCATTGTCTT
GCTTATCTACTGACTCTGCTTCATCAACAACAACAACAACAACCTCGGTTTCTTGTAACTATTT
GGTCTGA
-Exemplary Arabidopsis thaliana AP2/ERWEBP TF (Wrinkled 1 isoform
4 and isoform 5) Amino Acid Sequence
SEQ ID NO: 125
MILFVLIKIWTLTDKFGFFDLLFDLNFVTTSPVYLGAYDSEEAAAHTYDLAALKYWGPDTILNF
PAETYTKELEEMQRVTKEEYLASLRRQSSGFSRGVSKYRGVARHHHNGRWEARIGRVFGNKYLY
LGTYNTQEEAAAAYDMAAIEYRGANAVTNFDISNYIDRLKKKGVFPFPVNQANHQEGILVEAKQ
EVETREAKEEPREEVKQQYVEEPPQEEEEKEEEKAEQQEAEIVGYSEEAAVVNCCIDSSTIMEM
DRCGDNNELAWNFCMMDTGFSPFLTDQNLANENPIEYPELFNELAFEDNIDFMEDDGKHECLNL
ENLDCCVVGRESPPSSSSPLSCLSTDSASSTTTTTTSVSCNYLV
-Exemplary Arabidopsis thaliana AP2/ERF-type transcriptional activator
(Wrinkled 4 isoform 1) Nucleotide Coding Sequence
SEQ ID NO: 126
ATGGCAAAAGTCTCTGGGAGGAGCAAGAAAACAATCGTTGACGATGAAATCAGCGATAAAACAG
CGTCTGCGTCTGAGTCTGCGTCCATTGCCTTAACATCCAAACGCAAACGTAAGTCGCCGCCTCG
AAACGCTCCTCTTCAACGCAGCTCCCCTTACAGAGGCGTCACAAGGCATAGATGGACTGGGAGA
TACGAAGCGCATTTGTGGGATAAGAACAGCTGGAACGATACACAGACCAAGAAAGGACGTCAAG
TTTATCTAGGGGCTTACGACGAAGAAGAAGCAGCAGCACGTGCCTACGACTTAGCAGCATTGAA
GTACTGGGGACGAGACACACTCTTGAACTTCCCTTTGCCGAGTTATGACGAAGACGTCAAAGAA
ATGGAAGGCCAATCCAAGGAAGAGTATATTGGATCATTGAGAAGAAAAAGTAGTGGATTTTCTC
GCGGTGTATCAAAATACAGAGGCGTTGCAAGGCATCACCATAATGGGAGATGGGAAGCTAGAAT
TGGAAGGGTGTTTGCCACGCAAGAAGAAGCAGCAATCGCCTACGACATCGCGGCAATAGAGTAC
CGTGGACTTAACGCCGTTACCAATTTCGACGTCAGCCGTTATCTAAACCCTAACGCCGCCGCGG
ATAAAGCCGATTCCGATTCTAAGCCCATTCGAAGCCCTAGTCGCGAGCCCGAATCGTCGGATGA
TAACAAATCTCCGAAATCAGAGGAAGTAATCGAACCATCTACATCGCCGGAAGTGATTCCAACT
CGCCGGAGCTTCCCCGACGATATCCAGACGTATTTTGGGTGTCAAGATTCCGGCAAGTTAGCGA
CTGAGGAAGACGTAATATTCGATTGTTTCAATTCTTATATAAATCCTGGCTTCTATAACGAGTT
TGATTATGGACCTTAA
-Exemplary Arabidopsis thaliana AP2/ERF-type transcriptional activator
(Wrinkled 4 isoform 1) Amino Acid Sequence
SEQ ID NO: 127
MAKVSGRSKKTIVDDEISDKTASASESASIALTSKRKRKSPPRNAPLQRSSPYRGVTRHRWTGR
YEAHLWDKNSWNDTQTKKGRQVYLGAYDEEEAAARAYDLAALKYWGRDTLLNFPLPSYDEDVKE
MEGQSKEEYIGSLRRKSSGFSRGVSKYRGVARHHHNGRWEARIGRVFATQEEAAIAYDIAAIEY
RGLNAVTNFDVSRYLNPNAAADKADSDSKPIRSPSREPESSDDNKSPKSEEVIEPSTSPEVIPT
RRSFPDDIQTYFGCQDSGKLATEEDVIFDCENSYINPGFYNEFDYGP
-Exemplary Arabidopsis thaliana AP2/ERF-type transcriptional activator
(Wrinkled 4 isoform 2) Nucleotide Coding Sequence
SEQ ID NO: 128
ATGGCAAAAGTCTCTGGGAGGAGCAAGAAAACAATCGTTGACGATGAAATCAGCGATAAAACAG
CGTCTGCGTCTGAGTCTGCGTCCATTGCCTTAACATCCAAACGCAAACGTAAGTCGCCGCCTCG
AAACGCTCCTCTTCAACGCAGCTCCCCTTACAGAGGCGTCACAAGGCATAGATGGACTGGGAGA
TACGAAGCGCATTTGTGGGATAAGAACAGCTGGAACGATACACAGACCAAGAAAGGACGTCAAG
TTTATCTAGGGGCTTACGACGAAGAAGAAGCAGCAGCACGTGCCTACGACTTAGCAGCATTGAA
GTACTGGGGACGAGACACACTCTTGAACTTCCCTTTGCCGAGTTATGACGAAGACGTCAAAGAA
ATGGAAGGCCAATCCAAGGAAGAGTATATTGGATCATTGAGAAGAAAAAGTAGTGGATTTTCTC
GCGGTGTATCAAAATACAGAGGCGTTGCAAGGCATCACCATAATGGGAGATGGGAAGCTAGAAT
TGGAAGGGTGTTTGGTAATAAATATCTATATCTTGGAACATACGCCACGCAAGAAGAAGCAGCA
ATCGCCTACGACATCGCGGCAATAGAGTACCGTGGACTTAACGCCGTTACCAATTTCGACGTCA
GCCGTTATCTAAACCCTAACGCCGCCGCGGATAAAGCCGATTCCGATTCTAAGCCCATTCGAAG
CCCTAGTCGCGAGCCCGAATCGTCGGATGATAACAAATCTCCGAAATCAGAGGAAGTAATCGAA
CCATCTACATCGCCGGAAGTGATTCCAACTCGCCGGAGCTTCCCCGACGATATCCAGACGTATT
TTGGGTGTCAAGATTCCGGCAAGTTAGCGACTGAGGAAGACGTAATATTCGATTGTTTCAATTC
TTATATAAATCCTGGCTTCTATAACGAGTTTGATTATGGACCTTAA
-Exemplary Arabidopsis thaliana AP2/ERF-type transcriptional activator
(Wrinkled 4 isoform 2) Amino Acid Sequence
SEQ ID NO: 129
MAKVSGRSKKTIVDDEISDKTASASESASIALTSKRKRKSPPRNAPLQRSSPYRGVTRHRWTGR
YEAHLWDKNSWNDTQTKKGRQVYLGAYDEEEAAARAYDLAALKYWGRDTLLNFPLPSYDEDVKE
MEGQSKEEYIGSLRRKSSGFSRGVSKYRGVARHHHNGRWEARIGRVFGNKYLYLGTYATQEEAA
IAYDIAAIEYRGLNAVTNFDVSRYLNPNAAADKADSDSKPIRSPSREPESSDDNKSPKSEEVIE
PSTSPEVIPTRRSFPDDIQTYFGCQDSGKLATEEDVIFDCENSYINPGFYNEFDYGP
-Exemplary Arabidopsis thaliana AP2/ERF-type transcriptional activator
(Wrinkled 4 isoform 3) Nucleotide Coding Sequence
SEQ ID NO: 130
ATGATGAATGCTGACTCATCAAGTGCAGTTTATCTAGGGGCTTACGACGAAGAAGAAGCAGCAG
CACGTGCCTACGACTTAGCAGCATTGAAGTACTGGGGACGAGACACACTCTTGAACTTCCCTTT
GCCGAGTTATGACGAAGACGTCAAAGAAATGGAAGGCCAATCCAAGGAAGAGTATATTGGATCA
TTGAGAAGAAAAAGTAGTGGATTTTCTCGCGGTGTATCAAAATACAGAGGCGTTGCAAGGCATC
ACCATAATGGGAGATGGGAAGCTAGAATTGGAAGGGTGTTTGGTAATAAATATCTATATCTTGG
AACATACGCCACGCAAGAAGAAGCAGCAATCGCCTACGACATCGCGGCAATAGAGTACCGTGGA
CTTAACGCCGTTACCAATTTCGACGTCAGCCGTTATCTAAACCCTAACGCCGCCGCGGATAAAG
CCGATTCCGATTCTAAGCCCATTCGAAGCCCTAGTCGCGAGCCCGAATCGTCGGATGATAACAA
ATCTCCGAAATCAGAGGAAGTAATCGAACCATCTACATCGCCGGAAGTGATTCCAACTCGCCGG
AGCTTCCCCGACGATATCCAGACGTATTTTGGGTGTCAAGATTCCGGCAAGTTAGCGACTGAGG
AAGACGTAATATTCGATTGTTTCAATTCTTATATAAATCCTGGCTTCTATAACGAGTTTGATTA
TGGACCTTAA
-Exemplary Arabidopsis thaliana AP2/ERF-type transcriptional activator
(Wrinkled 4 isoform 3) Amino Acid Sequence
SEQ ID NO: 131
MMNADSSSAVYLGAYDEEEAAARAYDLAALKYWGRDTLLNFPLPSYDEDVKEMEGQSKEEYIGS
LRRKSSGFSRGVSKYRGVARHHHNGRWEARIGRVFGNKYLYLGTYATQEEAAIAYDIAAIEYRG
LNAVTNFDVSRYLNPNAAADKADSDSKPIRSPSREPESSDDNKSPKSEEVIEPSTSPEVIPTRR
SFPDDIQTYFGCQDSGKLATEEDVIFDCFENSYINPGFYNEFDYGP
-Exemplary Arabidopsis thaliana AP2/ERF-type transcriptional activator
(Wrinkled 4 isoform 4) Nucleotide Coding Sequence
SEQ ID NO: 132
ATGAATTCCACCGAAATTGGGGCTTACGACGAAGAAGAAGCAGCAGCACGTGCCTACGACTTAG
CAGCATTGAAGTACTGGGGACGAGACACACTCTTGAACTTCCCTTTGCCGAGTTATGACGAAGA
CGTCAAAGAAATGGAAGGCCAATCCAAGGAAGAGTATATTGGATCATTGAGAAGAAAAAGTAGT
GGATTTTCTCGCGGTGTATCAAAATACAGAGGCGTTGCAAGGCATCACCATAATGGGAGATGGG
AAGCTAGAATTGGAAGGGTGTTTGGTAATAAATATCTATATCTTGGAACATACGCCACGCAAGA
AGAAGCAGCAATCGCCTACGACATCGCGGCAATAGAGTACCGTGGACTTAACGCCGTTACCAAT
TTCGACGTCAGCCGTTATCTAAACCCTAACGCCGCCGCGGATAAAGCCGATTCCGATTCTAAGC
CCATTCGAAGCCCTAGTCGCGAGCCCGAATCGTCGGATGATAACAAATCTCCGAAATCAGAGGA
AGTAATCGAACCATCTACATCGCCGGAAGTGATTCCAACTCGCCGGAGCTTCCCCGACGATATC
CAGACGTATTTTGGGTGTCAAGATTCCGGCAAGTTAGCGACTGAGGAAGACGTAATATTCGATT
GTTTCAATTCTTATATAAATCCTGGCTTCTATAACGAGTTTGATTATGGACCTTAA
-Exemplary Arabidopsis thaliana AP2/ERF-type transcriptional activator
(Wrinkled 4 isoform 4) Amino Acid Sequence
SEQ ID NO: 133
MNSTEIGAYDEEEAAARAYDLAALKYWGRDTLLNFPLPSYDEDVKEMEGQSKEEYIGSLRRKSS
GFSRGVSKYRGVARHHHNGRWEARIGRVFGNKYLYLGTYATQEEAAIAYDIAAIEYRGLNAVTN
FDVSRYLNPNAAADKADSDSKPIRSPSREPESSDDNKSPKSEEVIEPSTSPEVIPTRRSFPDDI
QTYFGCQDSGKLATEEDVIFDCFNSYINPGFYNEFDYGP

### Hd-ZIP IV Leucine Zipper TF (WOOLLY)

In some embodiments, a composition described herein comprises a transgenic HD-Zip IV transcription factor. Such a transcription factor, among other things, is known to positively regulate CER6 transcription (a multicellular trichome regulator).

In some embodiments, a HD-Zip IV transcription factor gene and/or transgene comprises a sequence encoding a peptide sequence that is at least 90%, 91%, 92%, 93%, 94%, 95%, 96%, 97%, 98%, 99%, or 100% identical to SEQ ID NO: 135 (or a portion thereof). In some embodiments, a HD-Zip IV transcription factor gene and/or transgene comprises a nucleotide sequence that is at least 90%, 91%, 92%, 93%, 94%, 95%, 96%, 97%, 98%, 99%, or 100% identical to SEQ ID NO: 134 (or a portion thereof).

-Exemplary Solanum lycopersicum HD-ZIP IV leucine zipper TF
(Woolly, aka Protodermal factor 2) Nucleic Acid Coding Sequence
SEQ ID NO: 134
ATGTTTAATAACCACCAGCACTTGCTCGATATATCGTCCTCAGCTCAACGAACACCTGATAACG
AGTTGGATTTCATTCGTGATGAAGAGTTTGATAGCAACTCTGGTGCTGATAACATGGAAGCTCC
CAATTCAGGTGATGACGATCAAGCTGATCCAAACCAACCTCCAAACAAGAAGAAGCGTTATCAT
CGCCACACTCAGAATCAGATTCAGGAAATGGAGTCCTTTTACAAGGAATGCAATCATCCAGATG
ACAAGCAAAGGAAGGAATTGGGAAGAAGACTTGGTTTGGAGCCATTACAAGTGAAATTTTGGTT
CCAGAACAAGCGTACTCAGATGAAGGCTCAACATGAGCGATGTGAGAACACACAGTTGAGGAAT
GAAAATGAGAAGCTTCGCGCTGAGAACATAAGGTACAAAGAAGCTTTGAGTAATGCAGCATGCC
CAAATTGTGGAGGGCCAGCAGCTATAGGAGAGATGTCATTTGATGAGCATCAGTTGAGGATTGA
AAATGCTCGTCTTAGAGATGAGATTGACAGGATAACTGGAATAGCTGGAAAGTATGTTGGTAAA
TCAGCCCTTGGATATTCTCATCAACTTCCTCTTCCTCAGCCCGAAGCTCCTCGGGTTCTGGATC
TTGCTTTTGGGCCTCAATCGGGCCTGCTTGGAGAAATGTACGCTGCTGGTGACCTTCTAAGAAC
TGCTGTTACGGGCCTTACAGATGCTGAGAAGCCCGTGGTCATTGAGCTTGCTGTTACTGCAATG
GAGGAACTTATAAGGATGGCTCAAACTGAAGAGCCATTATGGTTGCCAAGCTCAGGCTCTGAGA
CTTTATGTGAGCAAGAATATGCTCGTATTTTCCCTCGAGGCCTTGGACCTAAGCCAGCTACACT
CAATTCTGAAGCCTCACGAGAATCTGCTGTTGTGATTATGAATCATATCAATTTAGTTGAGATT
TTGATGGATGTGAACCAATGGACTACTGTTTTTGCTGGTCTGGTGTCAAAAGCAATGACTCTTG
AAGTCTTATCAACTGGTGTCGCAGGAAATCACAATGGAGCATTGCAAGTGATGACAGCAGAATT
TCAAGTTCCATCTCCACTTGTTCCAACTCGGGAGAACTATTTCTTAAGATACTGTAAACAACAT
GGTGAAGGGACTTGGGTAGTGGTTGATGTTTCCCTGGACAACTTGCGCACTGTTTCAGTTCCGC
GTTGCAGAAGAAGGCCATCTGGTTGTTTAATCCAAGAAATGCCAAATGGTTACTCAAGGGTTAT
ATGGGTTGAACACGTTGAGGTGGATGAAAATGCTGTCCATGACATCTACAAACCTCTTGTCAAT
TCTGGGATTGCATTTGGAGCAAAACGCTGGGTAGCAACTTTAGATAGACAATGTGAACGCCTTG
CAAGTGTGTTGGCGCTTAACATCCCAACAGGAGATGTTGGAATCATTACTAGTCCAGCTGGTCG
AAAGAGTATGCTAAAACTTGCTGAGAGAATGGTGATGAGCTTTTGTGCTGGAGTTGGTGCATCG
ACAACTCACATATGGACAACTTTGTCTGGAAGTGGTGCGGATGATGTTAGAGTCATGACTAGGA
AGAGTATCGATGATCCAGGGAGACCTCCTGGTATTGTGCTGAGTGCTGCAACATCTTTTTGGCT
TCCAGTTTCTCCTAAGAGAGTGTTTGATTTTCTCCGCGATGAGAACTCTAGAAATGAGTGGGAT
ATTCTTTCAAATGGTGGGATTGTTCAGGAAATGGCACACATTGCAAATGGTCGTGATCCAGGAA
ACTGTGTTTCTCTACTCCGTGTCAATACTGGAACAAACTCTAACCAGAGTAACATGCTGATACT
CCAAGAGAGCACAACTGATGTAACAGGATCTTACGTCATTTACGCTCCAGTTGATATTGCTGCA
ATGAACGTGGTGTTAGGTGGGGGTGACCCTGACTATGTTGCTCTGTTGCCATCTGGTTTTGCTA
TTCTTCCAGACGGACCGATGAATTATCATGGTGGAGGTAATTCAGAAATTGATTCTCCTGGTGG
ATCGCTACTAACTGTAGCATTTCAGATATTGGTTGATTCAGTCCCAACTGCAAAGCTTTCCCTT
GGCTCTGTTGCGACTGTTAATAGTCTCATCAAATGCACCGTTGAAAAGATCAAAGGTGCTGTAA
CTTCCGCAAATGCATGA
-Exemplary Solanum lycopersicum HD-ZIP IV leucine zipper TF (woolly
aka Protodermal factor 2) Amino Acid Sequence
SEQ ID NO: 135
MFNNHQHLLDISSSAQRTPDNELDFIRDEEFDSNSGADNMEAPNSGDDDQADPNQPPNKKKRYH
RHTQNQIQEMESFYKECNHPDDKQRKELGRRLGLEPLQVKFWFQNKRTQMKAQHERCENTQLRN
ENEKLRAENIRYKEALSNAACPNCGGPAAIGEMSFDEHQLRIENARLRDEIDRITGIAGKYVGK
SALGYSHQLPLPQPEAPRVLDLAFGPQSGLLGEMYAAGDLLRTAVTGLTDAEKPVVIELAVTAM
EELIRMAQTEEPLWLPSSGSETLCEQEYARIFPRGLGPKPATLNSEASRESAVVIMNHINLVEI
LMDVNQWTTVFAGLVSKAMTLEVLSTGVAGNHNGALQVMTAEFQVPSPLVPTRENYFLRYCKQH
GEGTWVVVDVSLDNLRTVSVPRCRRRPSGCLIQEMPNGYSRVIWVEHVEVDENAVHDIYKPLVN
SGIAFGAKRWVATLDRQCERLASVLALNIPTGDVGIITSPAGRKSMLKLAERMVMSFCAGVGAS
TTHIWTTLSGSGADDVRVMTRKSIDDPGRPPGIVLSAATSFWLPVSPKRVFDFLRDENSRNEWD
ILSNGGIVQEMAHIANGRDPGNCVSLLRVNTGTNSNQSNMLILQESTTDVTGSYVIYAPVDIAA
MNVVLGGGDPDYVALLPSGFAILPDGPMNYHGGGNSEIDSPGGSLLTVAFQILVDSVPTAKLSL
GSVATVNSLIKCTVEKIKGAVTSANA

### Modifying Trichome Development

The present disclosure recognizes that in certain embodiments, modified trichome development may be useful for altering pollutant uptake. In some embodiments, compositions and methods of the present disclosure comprise modified (e.g., increased) levels of trichome development and/or total number. In some embodiments, such a modification is facilitated through transgene introduction, gene knockdown, and/or gene knockout using materials and methods described herein.

### R2R3 MYB Transcription Factor (MYB123-Like)

In some embodiments, a composition described herein comprises a transgenic R2R3 MYB transcription factor. Such a protein, among other things, may regulate different biological processes, such as primary and secondary metabolism, responses to biotic and abiotic stresses, developmental processes, and hormonal responses.

In some embodiments, a R2R3 MYB transcription factor gene and/or transgene comprises a sequence encoding a peptide sequence that is at least 90%, 91%, 92%, 93%, 94%, 95%, 96%, 97%, 98%, 99%, or 100% identical to SEQ ID NO: 137 (or a portion thereof). In some embodiments, a R2R3 MYB transcription factor gene and/or transgene comprises a nucleotide sequence that is at least 90%, 91%, 92%, 93%, 94%, 95%, 96%, 97%, 98%, 99%, or 100% identical to SEQ ID NO: 136 (or a portion thereof).

-Exemplary Nicotiana tomentosiformis R2R3 MYB transcription factor
(MYB123-Like) Nucleic Acid Coding Sequence
SEQ ID NO: 136
ATGGGAAGAAAGCCTTGTTGTTCTAAAGAAGGATTAAACAAAGGGGCATGGACTCCTATGGAGG
ATAAAATTCTAATAGATTATATCAAAGTAAATGGTGAAGGGAAATGGAGAAATCTTCCCAAAAG
AGCTGGTCTTAAAAGATGTGGAAAGAGTTGCAGACTAAGGTGGCTGAATTATCTAAGGCCAGAC
ATTAAGAGGGGAAATATAACTCCAGATGAAGAAGATCTCATTATCAGACTTCATAAACTTCTTG
GAAATAGATGGTCTCTGATAGCTGGAAGGCTACCAGGACGAACAGACAATGAAATCAAGAATTA
TTGGAACACAAACATCGGCAAAAAACTACAACAAGGAGTTGCTCCTGGTCAGCCAAACCGCATA
ATATCTTCCATTAATCGTCAGCGCCCTCGTTCTAGTCATGCCAAATCTTCCAAGTCCGACCCAG
TTACCCAACCAAACAAAAATAATCAAGAACACACAGTTCCTAATCAGGATTCACATTATTTGCT
AACAGACGTTGGATTCGGAGGATCATCGTCTTCTTCATCCCCGTGTTTGGTTATCCGCACAAAG
GCAATTAGGTGCACTAAAGTTTTTATTACTCCTCCTCCTACTAGTAGTTCGGTTGCTGAGCCAC
AGAATGTTGATCAGTCTCACAATGAGATTGCTCAAAGGGCTAGTAATTCTCACTCAGTCTTCCC
ACCTTGCACCAGGAATCCCGTTGAGTTCTTACGCTTTCATGTTGACAACTCAATTCTTGATAAT
GATAACGATGACAAGGTAATGGCGGAGGATTTGACAATAGAAAATGCAAATACTATTGTAGCAT
CGTCCTCATCATCGTCATCATTATCAGTGTCATCTTTGTCCGAGCAGCAACAACCAATATCAGG
ATCAAAACCAACTTTCTATGGAGAATTGGAAAATTATAACTTTAATTTTATGTTTGGTTTTGAT
ATGGACGATCCTTTTCTTTCTGAGCTTCTAAATGCACCTGATATATGTGAAAACTTGGAGAATA
CAACTACTGTTGGAGATAGTTGCAGCAAAAACGAAAAGGAAAGGAGCTATTTCCCTTCGAATTA
TAGTCAAACAACATTGTTCGCAGAAGATACGCAACACAACGATTTGGAACTTTGGATTAATGGG
TTCTCCTCTTGA
-Exemplary Nicotiana tomentosiformis R2R3 MYB transcription factor
(MYB123-Like) Amino Acid Sequence
SEQ ID NO: 137
MGRKPCCSKEGLNKGAWTPMEDKILIDYIKVNGEGKWRNLPKRAGLKRCGKSCRLRWLNYLRPD
IKRGNITPDEEDLIIRLHKLLGNRWSLIAGRLPGRTDNEIKNYWNTNIGKKLQQGVAPGQPNRI
ISSINRQRPRSSHAKSSKSDPVTQPNKNNQEHTVPNQDSHYLLTDVGFGGSSSSSSPCLVIRTK
AICTKVFITPPPTSSSVAEPQNVDQSHNEIAQRASNSHSVFPPCTRNPVEFLRFHVDNSILDND
NDDKVMAEDLTIENANTIVASSSSSSSLSVSSLSEQQQPISGSKPTFYGELENYNFNFMFGFDM
DDPFLSELLNAPDICENLENTTTVGDSCSKNEKERSYFPSNYSQTTLFAEDTQHNDLELWINGF
SS

### GLABRA1

In some embodiments, a composition described herein comprises a transgenic GLABRA1), encoded by the gene GLI, that creates the protein Trichome Differentiation protein GL1 a Myb-like protein. Such a protein, among other things, may regulate trichome differentiation.

In some embodiments, a GLABRA1 gene and/or transgene comprises a sequence encoding a peptide sequence that is at least 90%, 91%, 92%, 93%, 94%, 95%, 96%, 97%, 98%, 99%, or 100% identical to SEQ ID NO: 139 (or a portion thereof). In some embodiments, a GLABRA1 gene and/or transgene comprises a nucleotide sequence that is at least 90%, 91%, 92%, 93%, 94%, 95%, 96%, 97%, 98%, 99%, or 100% identical to SEQ ID NO: 138 (or a portion thereof).

-Exemplary Arabidopsis thaliana Myb-like TF (Glabrous 1) Nucleic Acid
Coding Sequence
SEQ ID NO: 138
ATGAGAATAAGGAGAAGAGATGAAAAAGAGAATCAAGAATACAAGAAAGGTTTATGGACAGTTG
AAGAAGACAACATCCTTATGGACTATGTTCTTAATCATGGCACTGGCCAATGGAACCGCATCGT
CAGAAAAACTGGGCTAAAGAGATGTGGGAAAAGTTGTAGACTGAGATGGATGAATTATTTGAGC
CCTAATGTGAACAAAGGCAATTTCACTGAACAAGAAGAAGACCTCATTATTCGTCTCCACAAGC
TCCTCGGCAATAGATGGTCTTTGATAGCTAAAAGAGTACCGGGAAGAACAGATAACCAAGTCAA
GAACTACTGGAACACTCATCTCAGCAAAAAACTCGTCGGAGATTACTCCTCCGCCGTCAAAACC
ACCGGAGAAGACGACGACTCTCCACCGTCATTGTTCATCACTGCCGCCACACCTTCTTCTTGTC
ATCATCAACAAGAAAATATCTACGAGAATATAGCCAAGAGCTTTAACGGCGTCGTATCAGCTTC
GTACGAGGATAAACCAAAACAAGAACTGGCTCAAAAAGATGTCCTAATGGCAACTACTAATGAT
CCAAGTCACTATTATGGCAATAACGCTTTATGGGTTCATGACGACGATTTTGAGCTTAGTTCAC
TCGTAATGATGAATTTTGCTTCTGGTGATGTTGAGTACTGCCTTTAG
-Exemplary Arabidopsis thaliana Myb-like TF (Glabrous 1) Amino Acid
Sequence
SEQ ID NO: 139
MRIRRRDEKENQEYKKGLWTVEEDNILMDYVLNHGTGQWNRIVRKTGLKRCGKSCRLRWMNYLS
PNVNKGNFTEQEEDLIIRLHKLLGNRWSLIAKRVPGRTDNQVKNYWNTHLSKKLVGDYSSAVKT
TGEDDDSPPSLFITAATPSSCHHQQENIYENIAKSFNGVVSASYEDKPKQELAQKDVLMATTND
PSHYYGNNALWVHDDDFELSSLVMMNFASGDVEYCL

### GLABRA2

In some embodiments, a composition described herein comprises a transgenic GLABRA2, encoded by the gene GL2. In certain embodiments, such a protein is an HD-ZIP IV family of homeobox-leucine zipper protein with lipid-binding START domain-containing protein. Such a protein, among other things, may regulate trichome differentiation.

In some embodiments, a GLABRA2 gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 141, 143, 145, 147, 149, or 151 (or a portion thereof). In some embodiments, a GLABRA2 gene and/or transgene comprises a nucleotide sequence that is at least 90%, 91%, 92%, 93%, 94%, 95%, 96%, 97%, 98%, 99%, or 100% identical to any one of SEQ ID NOs: 140, 142, 144, 146, 148, or 150 (or a portion thereof).

-Exemplary Arabidopsis thaliana HD-ZIP IV leucine zipper TF
(Glabrous 2 - Isoform 1) Nucleic Acid Coding Sequence
SEQ ID NO: 140
ATGAAGTCGATCGATGGCTGCCAATGCTGTAGCTGGCCATGTTTTAAACTACTCAATTCAAAGA
AGCTAGCTAGGGACAGGATTTGTATGTCAATGGCCGTCGACATGTCTTCCAAACAACCCACCAA
AGACTTTTTCTCCTCTCCAGCCCTCTCTCTATCTCTCGCTGGGATATTCCGGAATGCATCCTCC
GGCAGCACCAACCCTGAGGAGGATTTCCTGGGCAGAAGAGTAGTTGACGATGAGGATCGCACTG
TGGAGATGAGCAGCGAGAACTCAGGACCCACGAGATCCAGATCAGAGGAGGATTTGGAGGGTGA
GGATCACGACGATGAGGAGGAGGAAGAGGAGGACGGCGCAGCTGGAAACAAGGGCACTAATAAG
AGAAAGAGGAAGAAGTATCATCGTCACACCACCGATCAGATCAGACACATGGAAGCGCTATTCA
AAGAGACACCACATCCGGACGAGAAGCAAAGACAGCAGCTGAGCAAGCAACTAGGGCTGGCCCC
TCGCCAGGTCAAGTTCTGGTTCCAAAACCGCCGCACACAGATCAAGGCTATTCAAGAACGGCAC
GAGAACTCCCTGCTCAAGGCGGAACTAGAGAAGCTGCGAGAGGAAAACAAAGCCATGAGGGAGT
CTTTTTCCAAGGCTAATTCCTCCTGCCCCAACTGCGGAGGAGGCCCCGATGATCTCCACCTCGA
AAACTCCAAACTGAAAGCCGAGCTCGATAAGCTTCGTGCAGCTCTTGGACGCACTCCCTATCCC
CTGCAGGCTTCATGCTCCGACGATCAAGAACACCGTCTCGGCTCTCTCGATTTCTACACGGGCG
TCTTTGCCCTCGAGAAGTCCCGTATTGCCGAGATTTCTAACCGAGCCACCCTTGAACTCCAGAA
GATGGCCACCTCAGGCGAACCTATGTGGCTCCGCAGCGTTGAGACTGGCCGTGAGATTCTCAAC
TACGATGAGTACCTCAAGGAGTTTCCCCAAGCGCAAGCCTCTTCGTTTCCTGGAAGGAAAACCA
TCGAAGCATCTAGAGATGCGGGGATTGTGTTTATGGACGCACATAAACTTGCCCAGAGTTTCAT
GGACGTGGGACAATGGAAAGAGACATTTGCATGCTTGATCTCAAAGGCTGCAACGGTCGATGTT
ATCCGGCAAGGCGAAGGGCCTTCACGGATCGACGGGGCTATTCAGCTGATGTTCGGAGAGATGC
AGCTGCTCACTCCGGTCGTCCCCACAAGAGAAGTGTACTTCGTGAGAAGCTGCCGGCAGCTGAG
CCCTGAGAAATGGGCAATAGTGGACGTCTCGGTCTCCGTGGAGGACAGCAACACGGAGAAGGAG
GCTTCTCTTCTGAAATGTCGAAAACTCCCCTCCGGTTGCATCATCGAGGACACCTCCAACGGTC
ACTCCAAGGTCACCTGGGTGGAGCACCTCGACGTGTCTGCATCCACAGTTCAGCCTCTCTTCCG
CTCCTTAGTCAACACCGGTTTGGCCTTTGGGGCTCGACACTGGGTCGCCACCCTTCAGCTCCAT
TGCGAACGCCTTGTCTTCTTCATGGCTACCAACGTCCCCACCAAAGACTCTCTCGGAGTTACAA
CTCTTGCCGGGAGAAAGAGTGTGCTGAAGATGGCTCAGAGAATGACACAAAGCTTCTACCGCGC
CATTGCTGCATCAAGCTACCATCAATGGACCAAAATCACCACCAAAACTGGACAAGACATGCGG
GTTTCTTCCAGGAAGAACCTTCATGATCCTGGCGAGCCCACGGGAGTCATTGTCTGCGCTTCTT
CTTCGCTGTGGTTACCTGTTTCTCCAGCTCTTCTCTTCGATTTCTTTAGAGATGAAGCTCGTCG
GCATGAGTGGGATGCTTTGTCAAACGGAGCTCATGTTCAGTCTATTGCAAACTTATCCAAGGGA
CAAGACAGAGGCAACTCAGTGGCAATCCAGACAGTGAAATCGAGAGAAAAGAGCATATGGGTGC
TGCAAGACAGCAGCACTAACTCGTATGAGTCGGTGGTGGTATACGCTCCCGTAGATATAAACAC
GACACAGCTGGTGCTCGCGGGACATGATCCAAGCAACATCCAAATCCTCCCCTCTGGATTCTCA
ATCATACCTGATGGAGTAGAGTCACGGCCACTGGTAATAACGTCTACACAAGACGACAGAAACA
GCCAAGGAGGGTCGCTCCTGACACTCGCCCTCCAAACCCTCATCAACCCTTCTCCTGCAGCAAA
GCTGAATATGGAGTCTGTGGAATCCGTGACAAACCTCGTCTCAGTCACACTACACAACATTAAG
AGAAGTCTACAAATCGAAGATTGCTGA
-Exemplary Arabidopsis thaliana HD-ZIP IV leucine zipper TF
(Glabrous 2-Isoform 1) Amino Acid Sequence
SEQ ID NO: 141
MKSIDGCQCCSWPCFKLLNSKKLARDRICMSMAVDMSSKQPTKDFFSSPALSLSLAGIFRNASS
GSTNPEEDFLGRRVVDDEDRTVEMSSENSGPTRSRSEEDLEGEDHDDEEEEEEDGAAGNKGINK
RKRKKYHRHTTDQIRHMEALFKETPHPDEKQRQQLSKQLGLAPRQVKFWFQNRRTQIKAIQERH
ENSLLKAELEKLREENKAMRESFSKANSSCPNCGGGPDDLHLENSKLKAELDKLRAALGRTPYP
LQASCSDDQEHRLGSLDFYTGVFALEKSRIAEISNRATLELQKMATSGEPMWLRSVETGREILN
YDEYLKEFPQAQASSFPGRKTIEASRDAGIVFMDAHKLAQSFMDVGQWKETFACLISKAATVDV
IRQGEGPSRIDGAIQLMFGEMQLLTPVVPTREVYFVRSCRQLSPEKWAIVDVSVSVEDSNTEKE
ASLLKCRKLPSGCIIEDTSNGHSKVTWVEHLDVSASTVQPLFRSLVNTGLAFGARHWVATLQLH
CERLVFFMATNVPTKDSLGVTTLAGRKSVLKMAQRMTQSFYRAIAASSYHQWTKITTKTGQDMR
VSSRKNLHDPGEPTGVIVCASSSLWLPVSPALLFDFFRDEARRHEWDALSNGAHVQSIANLSKG
QDRGNSVAIQTVKSREKSIWVLQDSSTNSYESVVVYAPVDINTTQLVLAGHDPSNIQILPSGFS
IIPDGVESRPLVITSTQDDRNSQGGSLLTLALQTLINPSPAAKLNMESVESVINLVSVTLHNIK
RSLQIEDC
-Exemplary Arabidopsis thaliana HD-ZIP IV leucine zipper TF
(Glabrous 2-Isoform 2) Nucleic Acid Coding Sequence
SEQ ID NO: 142
ATGAGCAGCGAGAACTCAGGACCCACGAGATCCAGATCAGAGGAGGATTTGGAGGGTGAGGATC
ACGACGATGAGGAGGAGGAAGAGGAGGACGGCGCAGCTGGAAACAAGGGCACTAATAAGAGAAA
GAGGAAGAAGTATCATCGTCACACCACCGATCAGATCAGACACATGGAAGCGCTATTCAAAGAG
ACACCACATCCGGACGAGAAGCAAAGACAGCAGCTGAGCAAGCAACTAGGGCTGGCCCCTCGCC
AGGTCAAGTTCTGGTTCCAAAACCGCCGCACACAGATCAAGGCTATTCAAGAACGGCACGAGAA
CTCCCTGCTCAAGGCGGAACTAGAGAAGCTGCGAGAGGAAAACAAAGCCATGAGGGAGTCTTTT
TCCAAGGCTAATTCCTCCTGCCCCAACTGCGGAGGAGGCCCCGATGATCTCCACCTCGAAAACT
CCAAACTGAAAGCCGAGCTCGATAAGCTTCGTGCAGCTCTTGGACGCACTCCCTATCCCCTGCA
GGCTTCATGCTCCGACGATCAAGAACACCGTCTCGGCTCTCTCGATTTCTACACGGGCGTCTTT
GCCCTCGAGAAGTCCCGTATTGCCGAGATTTCTAACCGAGCCACCCTTGAACTCCAGAAGATGG
CCACCTCAGGCGAACCTATGTGGCTCCGCAGCGTTGAGACTGGCCGTGAGATTCTCAACTACGA
TGAGTACCTCAAGGAGTTTCCCCAAGCGCAAGCCTCTTCGTTTCCTGGAAGGAAAACCATCGAA
GCATCTAGAGATGCGGGGATTGTGTTTATGGACGCACATAAACTTGCCCAGAGTTTCATGGACG
TGGGACAATGGAAAGAGACATTTGCATGCTTGATCTCAAAGGCTGCAACGGTCGATGTTATCCG
GCAAGGCGAAGGGCCTTCACGGATCGACGGGGCTATTCAGCTGATGTTCGGAGAGATGCAGCTG
CTCACTCCGGTCGTCCCCACAAGAGAAGTGTACTTCGTGAGAAGCTGCCGGCAGCTGAGCCCTG
AGAAATGGGCAATAGTGGACGTCTCGGTCTCCGTGGAGGACAGCAACACGGAGAAGGAGGCTTC
TCTTCTGAAATGTCGAAAACTCCCCTCCGGTTGCATCATCGAGGACACCTCCAACGGTCACTCC
AAGGTCACCTGGGTGGAGCACCTCGACGTGTCTGCATCCACAGTTCAGCCTCTCTTCCGCTCCT
TAGTCAACACCGGTTTGGCCTTTGGGGCTCGACACTGGGTCGCCACCCTTCAGCTCCATTGCGA
ACGCCTTGTCTTCTTCATGGCTACCAACGTCCCCACCAAAGACTCTCTCGGAGTTACAACTCTT
GCCGGGAGAAAGAGTGTGCTGAAGATGGCTCAGAGAATGACACAAAGCTTCTACCGCGCCATTG
CTGCATCAAGCTACCATCAATGGACCAAAATCACCACCAAAACTGGACAAGACATGCGGGTTTC
TTCCAGGAAGAACCTTCATGATCCTGGCGAGCCCACGGGAGTCATTGTCTGCGCTTCTTCTTCG
CTGTGGTTACCTGTTTCTCCAGCTCTTCTCTTCGATTTCTTTAGAGATGAAGCTCGTCGGCATG
AGTGGGATGCTTTGTCAAACGGAGCTCATGTTCAGTCTATTGCAAACTTATCCAAGGGACAAGA
CAGAGGCAACTCAGTGGCAATCCAGACAGTGAAATCGAGAGAAAAGAGCATATGGGTGCTGCAA
GACAGCAGCACTAACTCGTATGAGTCGGTGGTGGTATACGCTCCCGTAGATATAAACACGACAC
AGCTGGTGCTCGCGGGACATGATCCAAGCAACATCCAAATCCTCCCCTCTGGATTCTCAATCAT
ACCTGATGGAGTAGAGTCACGGCCACTGGTAATAACGTCTACACAAGACGACAGAAACAGCCAA
GGAGGGTCGCTCCTGACACTCGCCCTCCAAACCCTCATCAACCCTTCTCCTGCAGCAAAGCTGA
ATATGGAGTCTGTGGAATCCGTGACAAACCTCGTCTCAGTCACACTACACAACATTAAGAGAAG
TCTACAAATCGAAGATTGCTGA
-Exemplary Arabidopsis thaliana HD-ZIP IV leucine zipper TF
(Glabrous 2-Isoform 2) Amino Acid Sequence
SEQ ID NO: 143
MSSENSGPTRSRSEEDLEGEDHDDEEEEEEDGAAGNKGTNKRKRKKYHRHTTDQIRHMEALFKE
TPHPDEKQRQQLSKQLGLAPRQVKFWFQNRRTQIKAIQERHENSLLKAELEKLREENKAMRESF
SKANSSCPNCGGGPDDLHLENSKLKAELDKLRAALGRTPYPLQASCSDDQEHRLGSLDFYTGVF
ALEKSRIAEISNRATLELQKMATSGEPMWLRSVETGREILNYDEYLKEFPQAQASSFPGRKTIE
ASRDAGIVFMDAHKLAQSFMDVGQWKETFACLISKAATVDVIRQGEGPSRIDGAIQLMFGEMQL
LTPVVPTREVYFVRSCRQLSPEKWAIVDVSVSVEDSNTEKEASLLKCRKLPSGCIIEDTSNGHS
KVTWVEHLDVSASTVQPLFRSLVNTGLAFGARHWVATLQLHCERLVFFMATNVPTKDSLGVTTL
AGRKSVLKMAQRMTQSFYRAIAASSYHQWTKITTKTGQDMRVSSRKNLHDPGEPTGVIVCASSS
LWLPVSPALLFDFFRDEARRHEWDALSNGAHVQSIANLSKGQDRGNSVAIQTVKSREKSIWVLQ
DSSTNSYESVVVYAPVDINTTQLVLAGHDPSNIQILPSGFSIIPDGVESRPLVITSTQDDRNSQ
GGSLLTLALQTLINPSPAAKLNMESVESVTNLVSVTLHNIKRSLQIEDC
-Exemplary Arabidopsis thaliana HD-ZIP IV leucine zipper TF
(Glabrous 2-Isoform 3) Nucleic Acid Coding Sequence
SEQ ID NO: 144
ATGTCAATGGCCGTCGACATGTCTTCCAAACAACCCACCAAAGACTTTTTCTCCTCTCCAGCCC
TCTCTCTATCTCTCGCTGGGATATTCCGGAATGCATCCTCCGGCAGCACCAACCCTGAGGAGGA
TTTCCTGGGCAGAAGAGTAGTTGACGATGAGGATCGCACTGTGGAGATGAGCAGCGAGAACTCA
GGACCCACGAGATCCAGATCAGAGGAGGATTTGGAGGGTGAGGATCACGACGATGAGGAGGAGG
AAGAGGAGGACGGCGCAGCTGGAAACAAGGGCACTAATAAGAGAAAGAGGAAGAAGTATCATCG
TCACACCACCGATCAGATCAGACACATGGAAGCGCTATTCAAAGAGACACCACATCCGGACGAG
AAGCAAAGACAGCAGCTGAGCAAGCAACTAGGGCTGGCCCCTCGCCAGGTCAAGTTCTGGTTCC
AAAACCGCCGCACACAGATCAAGGCTATTCAAGAACGGCACGAGAACTCCCTGCTCAAGGCGGA
ACTAGAGAAGCTGCGAGAGGAAAACAAAGCCATGAGGGAGTCTTTTTCCAAGGCTAATTCCTCC
TGCCCCAACTGCGGAGGAGGCCCCGATGATCTCCACCTCGAAAACTCCAAACTGAAAGCCGAGC
TCGATAAGCTTCGTGCAGCTCTTGGACGCACTCCCTATCCCCTGCAGGCTTCATGCTCCGACGA
TCAAGAACACCGTCTCGGCTCTCTCGATTTCTACACGGGCGTCTTTGCCCTCGAGAAGTCCCGT
ATTGCCGAGATTTCTAACCGAGCCACCCTTGAACTCCAGAAGATGGCCACCTCAGGCGAACCTA
TGTGGCTCCGCAGCGTTGAGACTGGCCGTGAGATTCTCAACTACGATGAGTACCTCAAGGAGTT
TCCCCAAGCGCAAGCCTCTTCGTTTCCTGGAAGGAAAACCATCGAAGCATCTAGAGATGCGGGG
ATTGTGTTTATGGACGCACATAAACTTGCCCAGAGTTTCATGGACGTGGGACAATGGAAAGAGA
CATTTGCATGCTTGATCTCAAAGGCTGCAACGGTCGATGTTATCCGGCAAGGCGAAGGGCCTTC
ACGGATCGACGGGGCTATTCAGCTGATGTTCGGAGAGATGCAGCTGCTCACTCCGGTCGTCCCC
ACAAGAGAAGTGTACTTCGTGAGAAGCTGCCGGCAGCTGAGCCCTGAGAAATGGGCAATAGTGG
ACGTCTCGGTCTCCGTGGAGGACAGCAACACGGAGAAGGAGGCTTCTCTTCTGAAATGTCGAAA
ACTCCCCTCCGGTTGCATCATCGAGGACACCTCCAACGGTCACTCCAAGGTCACCTGGGTGGAG
CACCTCGACGTGTCTGCATCCACAGTTCAGCCTCTCTTCCGCTCCTTAGTCAACACCGGTTTGG
CCTTTGGGGCTCGACACTGGGTCGCCACCCTTCAGCTCCATTGCGAACGCCTTGTCTTCTTCAT
GGCTACCAACGTCCCCACCAAAGACTCTCTCGGAGTTACAACTCTTGCCGGGAGAAAGAGTGTG
CTGAAGATGGCTCAGAGAATGACACAAAGCTTCTACCGCGCCATTGCTGCATCAAGCTACCATC
AATGGACCAAAATCACCACCAAAACTGGACAAGACATGCGGGTTTCTTCCAGGAAGAACCTTCA
TGATCCTGGCGAGCCCACGGGAGTCATTGTCTGCGCTTCTTCTTCGCTGTGGTTACCTGTTTCT
CCAGCTCTTCTCTTCGATTTCTTTAGAGATGAAGCTCGTCGGCATGAGTGGGATGCTTTGTCAA
ACGGAGCTCATGTTCAGTCTATTGCAAACTTATCCAAGGGACAAGACAGAGGCAACTCAGTGGC
AATCCAGACAGTGAAATCGAGAGAAAAGAGCATATGGGTGCTGCAAGACAGCAGCACTAACTCG
TATGAGTCGGTGGTGGTATACGCTCCCGTAGATATAAACACGACACAGCTGGTGCTCGCGGGAC
ATGATCCAAGCAACATCCAAATCCTCCCCTCTGGATTCTCAATCATACCTGATGGAGTAGAGTC
ACGGCCACTGGTAATAACGTCTACACAAGACGACAGAAACAGCCAAGGAGGGTCGCTCCTGACA
CTCGCCCTCCAAACCCTCATCAACCCTTCTCCTGCAGCAAAGCTGAATATGGAGTCTGTGGAAT
CCGTGACAAACCTCGTCTCAGTCACACTACACAACATTAAGAGAAGTCTACAAATCGAAGATTG
CTGA
-Exemplary Arabidopsis thaliana HD-ZIP IV leucine zipper TF
(Glabrous 2-Isoform 3) Amino Acid Sequence
SEQ ID NO: 145
MSMAVDMSSKQPTKDFFSSPALSLSLAGIFRNASSGSTNPEEDFLGRRVVDDEDRTVEMSSENS
GPTRSRSEEDLEGEDHDDEEEEEEDGAAGNKGTNKRKRKKYHRHTTDQIRHMEALFKETPHPDE
KQRQQLSKQLGLAPRQVKFWFQNRRTQIKAIQERHENSLLKAELEKLREENKAMRESFSKANSS
CPNCGGGPDDLHLENSKLKAELDKLRAALGRTPYPLQASCSDDQEHRLGSLDFYTGVFALEKSR
IAEISNRATLELQKMATSGEPMWLRSVETGREILNYDEYLKEFPQAQASSFPGRKTIEASRDAG
IVFMDAHKLAQSFMDVGQWKETFACLISKAATVDVIRQGEGPSRIDGAIQLMFGEMQLLTPVVP
TREVYFVRSCRQLSPEKWAIVDVSVSVEDSNTEKEASLLKCRKLPSGCIIEDTSNGHSKVTWVE
HLDVSASTVQPLFRSLVNTGLAFGARHWVATLQLHCERLVFFMATNVPTKDSLGVTTLAGRKSV
LKMAQRMTQSFYRAIAASSYHQWTKITTKTGQDMRVSSRKNLHDPGEPTGVIVCASSSLWLPVS
PALLFDFFRDEARRHEWDALSNGAHVQSIANLSKGQDRGNSVAIQTVKSREKSIWVLQDSSTNS
YESVVVYAPVDINTTQLVLAGHDPSNIQILPSGFSIIPDGVESRPLVITSTQDDRNSQGGSLLT
LALQTLINPSPAAKLNMESVESVTNLVSVTLHNIKRSLQIEDC
-Exemplary Arabidopsis thaliana HD-ZIP IV leucine zipper TF
(Glabrous 2-Isoform 4) Nucleic Acid Coding Sequence
SEQ ID NO: 146
ATGTCAATGGCCGTCGACATGTCTTCCAAACAACCCACCAAAGACTTTTTCTCCTCTCCAGCCC
TCTCTCTATCTCTCGCTGGGATATTCCGGAATGCATCCTCCGGCAGCACCAACCCTGAGGAGGA
TTTCCTGGGCAGAAGAGTAGTTGACGATGAGGATCGCACTGTGGAGATGAGCAGCGAGAACTCA
GGACCCACGAGATCCAGATCAGAGGAGGATTTGGAGGGTGAGGATCACGACGATGAGGAGGAGG
AAGAGGAGGACGGCGCAGCTGGAAACAAGGGCACTAATAAGAGAAAGAGGAAGAAGTATCATCG
TCACACCACCGATCAGATCAGACACATGGAAGCGCTATTCAAAGAGACACCACATCCGGACGAG
AAGCAAAGACAGCAGCTGAGCAAGCAACTAGGGCTGGCCCCTCGCCAGGTCAAGTTCTGGTTCC
AAAACCGCCGCACACAGATCAAGGCTATTCAAGAACGGCACGAGAACTCCCTGCTCAAGGCGGA
ACTAGAGAAGCTGCGAGAGGAAAACAAAGCCATGAGGGAGTCTTTTTCCAAGGCTAATTCCTCC
TGCCCCAACTGCGGAGGAGGCCCCGATGATCTCCACCTCGAAAACTCCAAACTGAAAGCCGAGC
TCGATAAGCTTCGTGCAGCTCTTGGACGCACTCCCTATCCCCTGCAGGCTTCATGCTCCGACGA
TCAAGAACACCGTCTCGGCTCTCTCGATTTCTACACGGGCGTCTTTGCCCTCGAGAAGTCCCGT
ATTGCCGAGATTTCTAACCGAGCCACCCTTGAACTCCAGAAGATGGCCACCTCAGGCGAACCTA
TGTGGCTCCGCAGCGTTGAGACTGGCCGTGAGATTCTCAACTACGATGAGTACCTCAAGGAGTT
TCCCCAAGCGCAAGCCTCTTCGTTTCCTGGAAGGAAAACCATCGAAGCATCTAGAGATGCGGGG
ATTGTGTTTATGGACGCACATAAACTTGCCCAGAGTTTCATGGACGTGGGACAATGGAAAGAGA
CATTTGCATGCTTGATCTCAAAGGCTGCAACGGTCGATGTTATCCGGCAAGGCGAAGGGCCTTC
ACGGATCGACGGGGCTATTCAGCTGATGTTCGGAGAGATGCAGCTGCTCACTCCGGTCGTCCCC
ACAAGAGAAGTGTACTTCGTGAGAAGCTGCCGGCAGCTGAGCCCTGAGAAATGGGCAATAGTGG
ACGTCTCGGTCTCCGTGGAGGACAGCAACACGGAGAAGGAGGCTTCTCTTCTGAAATGTCGAAA
ACTCCCCTCCGGTTGCATCATCGAGGACACCTCCAACGGTCACTCCAAGGTCACCTGGGTGGAG
CACCTCGACGTGTCTGCATCCACAGTTCAGCCTCTCTTCCGCTCCTTAGTCAACACCGGTTTGG
CCTTTGGGGCTCGACACTGGGTCGCCACCCTTCAGCTCCATTGCGAACGCCTTGTCTTCTTCAT
GGCTACCAACGTCCCCACCAAAGACTCTCTCGGAGTTACAACTCTTGCCGGGAGAAAGAGTGTG
CTGAAGATGGCTCAGAGAATGACACAAAGCTTCTACCGCGCCATTGCTGCATCAAGCTACCATC
AATGGACCAAAATCACCACCAAAACTGGACAAGACATGCGGGTTTCTTCCAGGAAGAACCTTCA
TGATCCTGGCGAGCCCACGGGAGTCATTGTCTGCGCTTCTTCTTCGCTGTGGTTACCTGTTTCT
CCAGCTCTTCTCTTCGATTTCTTTAGAGATGAAGCTCGTCGGCATGAGTGGGATGCTTTGTCAA
ACGGAGCTCATGTTCAGTCTATTGCAAACTTATCCAAGGGACAAGACAGAGGCAACTCAGTGGC
AATCCAGGTGCGTTTATTTTGTCTTCTCCTCCTCTAA
-Exemplary Arabidopsis thaliana HD-ZIP IV leucine zipper TF
(Glabrous 2-Isoform 4) Amino Acid Sequence
SEQ ID NO: 147
MSMAVDMSSKQPTKDFFSSPALSLSLAGIFRNASSGSTNPEEDFLGRRVVDDEDRIVEMSSENS
GPTRSRSEEDLEGEDHDDEEEEEEDGAAGNKGTNKRKRKKYHRHTTDQIRHMEALFKETPHPDE
KQRQQLSKQLGLAPRQVKFWFQNRRTQIKAIQERHENSLLKAELEKLREENKAMRESFSKANSS
CPNCGGGPDDLHLENSKLKAELDKLRAALGRTPYPLQASCSDDQEHRLGSLDFYTGVFALEKSR
IAEISNRATLELQKMATSGEPMWLRSVETGREILNYDEYLKEFPQAQASSFPGRKTIEASRDAG
IVFMDAHKLAQSFMDVGQWKETFACLISKAATVDVIRQGEGPSRIDGAIQLMFGEMQLLTPVVP
TREVYFVRSCRQLSPEKWAIVDVSVSVEDSNTEKEASLLKCRKLPSGCIIEDTSNGHSKVTWVE
HLDVSASTVQPLFRSLVNTGLAFGARHWVATLQLHCERLVFFMATNVPTKDSLGVTTLAGRKSV
LKMAQRMTQSFYRAIAASSYHQWTKITTKTGQDMRVSSRKNLHDPGEPTGVIVCASSSLWLPVS
PALLFDFFRDEARRHEWDALSNGAHVQSIANLSKGQDRGNSVAIQVRLFCLLLL
-Exemplary Arabidopsis thaliana HD-ZIP IV leucine zipper TF
(Glabrous 2-Isoform 5) Nucleic Acid Coding Sequence
SEQ ID NO: 148
ATGTCAATGGCCGTCGACATGTCTTCCAAACAACCCACCAAAGACTTTTTCTCCTCTCCAGCCC
TCTCTCTATCTCTCGCTGGGATATTCCGGAATGCATCCTCCGGCAGCACCAACCCTGAGGAGGA
TTTCCTGGGCAGAAGAGTAGTTGACGATGAGGATCGCACTGTGGAGATGAGCAGCGAGAACTCA
GGACCCACGAGATCCAGATCAGAGGAGGATTTGGAGGGTGAGGATCACGACGATGAGGAGGAGG
AAGAGGAGGACGGCGCAGCTGGAAACAAGGGCACTAATAAGAGAAAGAGGAAGAAGTATCATCG
TCACACCACCGATCAGATCAGACACATGGAAGCGCTATTCAAAGAGACACCACATCCGGACGAG
AAGCAAAGACAGCAGCTGAGCAAGCAACTAGGGCTGGCCCCTCGCCAGGTCAAGTTCTGGTTCC
AAAACCGCCGCACACAGATCAAGGCTATTCAAGAACGGCACGAGAACTCCCTGCTCAAGGCGGA
ACTAGAGAAGCTGCGAGAGGAAAACAAAGCCATGAGGGAGTCTTTTTCCAAGGCTAATTCCTCC
TGCCCCAACTGCGGAGGAGGCCCCGATGATCTCCACCTCGAAAACTCCAAACTGAAAGCCGAGC
TCGATAAGCTTCGTGCAGCTCTTGGACGCACTCCCTATCCCCTGCAGGCTTCATGCTCCGACGA
TCAAGAACACCGTCTCGGCTCTCTCGATTTCTACACGGGCGTCTTTGCCCTCGAGAAGTCCCGT
ATTGCCGAGATTTCTAACCGAGCCACCCTTGAACTCCAGAAGATGGCCACCTCAGGCGAACCTA
TGTGGCTCCGCAGCGTTGAGACTGGCCGTGAGATTCTCAACTACGATGAGTACCTCAAGGAGTT
TCCCCAAGCGCAAGCCTCTTCGTTTCCTGGAAGGAAAACCATCGAAGCATCTAGAGATGCGGGG
ATTGTGTTTATGGACGCACATAAACTTGCCCAGAGTTTCATGGACGTGGGACAATGGAAAGAGA
CATTTGCATGCTTGATCTCAAAGGCTGCAACGGTCGATGTTATCCGGCAAGGCGAAGGGCCTTC
ACGGATCGACGGGGCTATTCAGCTGATGTTCGGAGAGATGCAGCTGCTCACTCCGGTCGTCCCC
ACAAGAGAAGTGTACTTCGTGAGAAGCTGCCGGCAGCTGAGCCCTGAGAAATGGGCAATAGTGG
ACGTCTCGGTCTCCGTGGAGGACAGCAACACGGAGAAGGAGGCTTCTCTTCTGAAATGTCGAAA
ACTCCCCTCCGGTTGCATCATCGAGGACACCTCCAACGGTCACTCCAAGGTCACCTGGGTGGAG
CACCTCGACGTGTCTGCATCCACAGTTCAGCCTCTCTTCCGCTCCTTAGTCAACACCGGTTTGG
CCTTTGGGGCTCGACACTGGGTCGCCACCCTTCAGCTCCATTGCGAACGCCTTGTCTTCTTCAT
GGCTACCAACGTCCCCACCAAAGACTCTCTCGGTCCGTCTATATATCCGGATCCTCCATTTACA
CTCTCTATCTTTCTTTATATATAA
-Exemplary Arabidopsis thaliana HD-ZIP IV leucine zipper TF
(Glabrous 2-Isoform 5) Amino Acid Sequence
SEQ ID NO: 149
MSMAVDMSSKQPTKDFFSSPALSLSLAGIFRNASSGSTNPEEDFLGRRVVDDEDRIVEMSSENS
GPTRSRSEEDLEGEDHDDEEEEEEDGAAGNKGTNKRKRKKYHRHTTDQIRHMEALFKETPHPDE
KQRQQLSKQLGLAPRQVKFWFQNRRTQIKAIQERHENSLLKAELEKLREENKAMRESFSKANSS
CPNCGGGPDDLHLENSKLKAELDKLRAALGRTPYPLQASCSDDQEHRLGSLDFYTGVFALEKSR
IAEISNRATLELQKMATSGEPMWLRSVETGREILNYDEYLKEFPQAQASSFPGRKTIEASRDAG
IVFMDAHKLAQSFMDVGQWKETFACLISKAATVDVIRQGEGPSRIDGAIQLMFGEMQLLTPVVP
TREVYFVRSCRQLSPEKWAIVDVSVSVEDSNTEKEASLLKCRKLPSGCIIEDTSNGHSKVTWVE
HLDVSASTVQPLFRSLVNTGLAFGARHWVATLQLHCERLVFFMATNVPTKDSLGPSIYPDPPFT
LSIFLYI
-Exemplary Arabidopsis thaliana HD-ZIP IV leucine zipper TF
(Glabrous 2-Isoform 6) Nucleic Acid Coding Sequence
SEQ ID NO: 150
ATGAGCAGCGAGAACTCAGGACCCACGAGATCCAGATCAGAGGAGGATTTGGAGGGTGAGGATC
ACGACGATGAGGAGGAGGAAGAGGAGGACGGCGCAGCTGGAAACAAGGGCACTAATAAGAGAAA
GAGGAAGAAGTATCATCGTCACACCACCGATCAGATCAGACACATGGAAGCGCTATTCAAAGAG
ACACCACATCCGGACGAGAAGCAAAGACAGCAGCTGAGCAAGCAACTAGGGCTGGCCCCTCGCC
AGGTCAAGTTCTGGTTCCAAAACCGCCGCACACAGATCAAGGCTATTCAAGAACGGCACGAGAA
CTCCCTGCTCAAGGCGGAACTAGAGAAGCTGCGAGAGGAAAACAAAGCCATGAGGGAGTCTTTT
TCCAAGGCTAATTCCTCCTGCCCCAACTGCGGAGGAGGCCCCGATGATCTCCACCTCGAAAACT
CCAAACTGAAAGCCGAGCTCGATAAGCTTCGTGCAGCTCTTGGACGCACTCCCTATCCCCTGCA
GGCTTCATGCTCCGACGATCAAGAACACCGTCTCGGCTCTCTCGATTTCTACACGGGCGTCTTT
GCCCTCGAGAAGTCCCGTATTGCCGAGATTTCTAACCGAGCCACCCTTGAACTCCAGAAGATGG
CCACCTCAGGCGAACCTATGTGGCTCCGCAGCGTTGAGACTGGCCGTGAGATTCTCAACTACGA
TGAGTACCTCAAGGAGTTTCCCCAAGCGCAAGCCTCTTCGTTTCCTGGAAGGAAAACCATCGAA
GCATCTAGAGATGCGGGGATTGTGTTTATGGACGCACATAAACTTGCCCAGAGTTTCATGGACG
TGGGACAATGGAAAGAGACATTTGCATGCTTGATCTCAAAGGCTGCAACGGTCGATGTTATCCG
GCAAGGCGAAGGGCCTTCACGGATCGACGGGGCTATTCAGCTGATGTTCGGAGAGATGCAGCTG
CTCACTCCGGTCGTCCCCACAAGAGAAGTGTACTTCGTGAGAAGCTGCCGGCAGCTGAGCCCTG
AGAAATGGGCAATAGTGGACGTCTCGGTCTCCGTGGAGGACAGCAACACGGAGAAGGAGGCTTC
TCTTCTGAAATGTCGAAAACTCCCCTCCGGTTGCATCATCGAGGACACCTCCAACGGTCACTCC
AAGGTCACCTGGGTGGAGCACCTCGACGTGTCTGCATCCACAGTTCAGCCTCTCTTCCGCTCCT
TAGTCAACACCGGTTTGGCCTTTGGGGCTCGACACTGGGTCGCCACCCTTCAGCTCCATTGCGA
ACGCCTTGTCTTCTTCATGGCTACCAACGTCCCCACCAAAGACTCTCTCGGAGTTACAACTCTT
GCCGGGAGAAAGAGTGTGCTGAAGATGGCTCAGAGAATGACACAAAGCTTCTACCGCGCCATTG
CTGCATCAAGCTACCATCAATGGACCAAAATCACCACCAAAACTGGACAAGACATGCGGGTTTC
TTCCAGGAAGAACCTTCATGATCCTGGCGAGCCCACGGGAGTCATTGTCTGCGCTTCTTCTTCG
CTGTGGTTACCTGTTTCTCCAGCTCTTCTCTTCGATTTCTTTAGAGATGAAGCTCGTCGGCATG
AGTGGGATGCTTTGTCAAACGGAGCTCATGTTCAGTCTATTGCAAACTTATCCAAGGGACAAGA
CAGAGGCAACTCAGTGGCAATCCAGACAGTGAAATCGAGAGAAAAGAGCATATGGGTGCTGCAA
GACAGCAGCACTAACTCGTATGAGTCGGTGGTGGTATACGCTCCCGTAGATATAAACACGACAC
AGCTGGTGCTCGCGGGACATGATCCAAGCAACATCCAAATCCTCCCCTCTGGATTCTCAATCAT
ACCTGATGGAGTAGAGTCACGGCCACTGGTAATAACGTCTACACAAGACGACAGAAACAGCCAA
GGAGGGTCGCTCCTGACACTCGCCCTCCAAACCCTCATCAACCCTTCTCCTGCAGCAAAGCTGA
ATATGGAGTCTGTGGAATCCGTGACAAACCTCGTCTCAGTCACACTACACAACATTAAGAGAAG
TCTACAAATCGAAGATTGCTGA
-Exemplary Arabidopsis thaliana HD-ZIP IV leucine zipper TF
(Glabrous 2 - Isoform 6) Amino Acid Sequence
SEQ ID NO: 151
MSSENSGPTRSRSEEDLEGEDHDDEEEEEEDGAAGNKGTNKRKRKKYHRHTTDQIRHMEALFKE
TPHPDEKQRQQLSKQLGLAPRQVKFWFQNRRTQIKAIQERHENSLLKAELEKLREENKAMRESF
SKANSSCPNCGGGPDDLHLENSKLKAELDKLRAALGRTPYPLQASCSDDQEHRLGSLDFYTGVF
ALEKSRIAEISNRATLELQKMATSGEPMWLRSVETGREILNYDEYLKEFPQAQASSFPGRKTIE
ASRDAGIVFMDAHKLAQSFMDVGQWKETFACLISKAATVDVIRQGEGPSRIDGAIQLMFGEMQL
LTPVVPTREVYFVRSCRQLSPEKWAIVDVSVSVEDSNTEKEASLLKCRKLPSGCIIEDTSNGHS
KVTWVEHLDVSASTVQPLFRSLVNTGLAFGARHWVATLQLHCERLVFFMATNVPTKDSLGVTTL
AGRKSVLKMAQRMTQSFYRAIAASSYHQWTKITTKTGQDMRVSSRKNLHDPGEPTGVIVCASSS
LWLPVSPALLFDFFRDEARRHEWDALSNGAHVQSIANLSKGQDRGNSVAIQTVKSREKSIWVLQ
DSSTNSYESVVVYAPVDINTTQLVLAGHDPSNIQILPSGFSIIPDGVESRPLVITSTQDDRNSQ
GGSLLTLALQTLINPSPAAKLNMESVESVTNLVSVTLHNIKRSLQIEDC

### GLABRA3

In some embodiments, a composition described herein comprises a transgenic GLABRA3, encoded by the gene GL3. In some embodiments, such a protein, among other things, may regulate trichome differentiation.

In some embodiments, a GLABRA3 gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 153, 155, or 157 (or a portion thereof). In some embodiments, a GLABRA3 gene and/or transgene comprises a nucleotide sequence that is at least 90%, 91%, 92%, 93%, 94%, 95%, 96%, 97%, 98%, 99%, or 100% identical to any one of SEQ ID NOs: 152, 154, or 156 (or a portion thereof).

-Exemplary Arabidopsis thaliana Basic Helix Loop Helix domain TF
(Glabrous 3 - Isoform 1) Nucleic Acid Coding Sequence
SEQ ID NO: 152
ATGGGATATAGGGATGAAGAAACAATGGCTACCGGACAAAACAGAACAACTGTGCCAGAGAATC
TGAAGAAACACCTCGCAGTTTCAGTTCGAAACATTCAATGGAGTTATGGTATCTTTTGGTCTGT
CTCTGCTTCTCAGTCTGGAGTTTTAGAATGGGGAGATGGATACTATAATGGAGATATCAAAACG
AGGAAGACGATTCAAGCTTCGGAGATCAAAGCTGATCAGCTTGGTCTACGGAGGAGCGAGCAGC
TTAGCGAGCTTTACGAGTCTCTCTCCGTCGCTGAATCTTCTTCTTCAGGCGTTGCTGCCGGATC
TCAAGTCACCAGACGAGCTTCCGCCGCCGCACTTTCACCGGAAGATCTCGCCGACACCGAGTGG
TACTATTTGGTTTGTATGTCTTTCGTCTTCAACATTGGTGAAGGAATGCCTGGACGGACGTTTG
CAAACGGTGAACCGATATGGTTGTGCAACGCTCATACGGCGGATAGTAAAGTGTTTAGCCGTTC
TCTTCTAGCAAAAAGTGCTGCGGTTAAGACAGTGGTTTGCTTCCCGTTCCTTGGAGGAGTCGTT
GAGATTGGTACCACAGAACATATTACGGAAGACATGAATGTAATACAATGCGTGAAGACATCAT
TCCTCGAAGCCCCTGATCCGTACGCTACAATATTACCAGCAAGATCCGATTATCACATCGACAA
CGTTCTTGATCCGCAACAGATTCTAGGCGACGAGATTTACGCGCCTATGTTCAGTACGGAGCCT
TTTCCAACAGCTTCTCCGAGCAGAACTACCAACGGTTTCGATCAAGAACATGAACAAGTAGCAG
ATGATCATGATTCTTTCATGACCGAAAGAATCACTGGAGGAGCTTCTCAGGTGCAAAGCTGGCA
GCTCATGGACGACGAGCTTAGTAACTGCGTTCACCAGTCGCTAAATTCCAGCGATTGCGTCTCT
CAAACGTTTGTTGAAGGGGCGGCTGGACGGGTTGCTTACGGTGCAAGAAAGAGTAGAGTTCAAA
GACTAGGGCAAATTCAAGAGCAACAGAGAAATGTGAAGACATTGTCATTTGATCCAAGAAACGA
CGACGTTCATTACCAAAGTGTGATCTCAACGATTTTTAAGACCAACCATCAGTTAATTCTCGGA
CCGCAGTTTCGAAACTGCGATAAACAGTCAAGCTTCACTAGGTGGAAGAAATCATCGTCATCAT
CATCAGGAACCGCCACGGTCACGGCACCATCACAAGGAATGTTAAAGAAAATTATTTTCGATGT
TCCGCGAGTGCACCAGAAAGAGAAGTTAATGTTGGACTCACCAGAAGCCAGAGATGAAACTGGG
AACCATGCGGTTTTAGAGAAGAAGCGCCGCGAGAAATTGAACGAACGGTTCATGACCTTGAGAA
AAATCATTCCGTCAATCAACAAGATCGATAAAGTATCGATTCTTGACGATACGATAGAGTATCT
TCAAGAACTCGAGAGACGGGTTCAAGAACTAGAATCTTGCAGAGAATCAACCGATACAGAGACT
CGTGGGACGATGACGATGAAGAGGAAGAAACCATGCGACGCAGGAGAAAGAACATCAGCTAATT
GCGCAAATAATGAAACAGGAAATGGGAAGAAGGTGTCGGTTAACAATGTTGGTGAAGCCGAGCC
AGCAGATACCGGTTTTACTGGTTTAACCGATAATTTAAGGATCGGTTCGTTTGGTAATGAGGTG
GTTATTGAGCTTAGATGTGCTTGGAGAGAAGGAGTATTGCTTGAGATAATGGATGTGATTAGTG
ATCTCCATTTGGATTCTCATTCGGTTCAATCCTCGACCGGAGACGGTTTGCTCTGCTTAACCGT
CAATTGCAAGCACAAGGGGTCAAAAATAGCGACACCAGGAATGATCAAAGAAGCACTTCAAAGG
GTTGCATGGATCTGTTGA
-Exemplary Arabidopsis thaliana Basic Helix Loop Helix domain TF
(Glabrous 3-Isoform 1) Amino Acid Sequence
SEQ ID NO: 153
MATGQNRTTVPENLKKHLAVSVRNIQWSYGIFWSVSASQSGVLEWGDGYYNGDIKTRKTIQASE
IKADQLGLRRSEQLSELYESLSVAESSSSGVAAGSQVTRRASAAALSPEDLADTEWYYLVCMSF
VFNIGEGMPGRTFANGEPIWLCNAHTADSKVFSRSLLAKSAAVKTVVCFPFLGGVVEIGTTEHI
TEDMNVIQCVKTSFLEAPDPYATILPARSDYHIDNVLDPQQILGDEIYAPMFSTEPFPTASPSR
TTNGFDQEHEQVADDHDSFMTERITGGASQVQSWQLMDDELSNCVHQSLNSSDCVSQTFVEGAA
GRVAYGARKSRVQRLGQIQEQQRNVKTLSFDPRNDDVHYQSVISTIFKINHQLILGPQFRNCDK
QSSFTRWKKSSSSSSGTATVTAPSQGMLKKIIFDVPRVHQKEKLMLDSPEARDETGNHAVLEKK
RREKLNERFMTLRKIIPSINKIDKVSILDDTIEYLQELERRVQELESCRESTDTETRGTMTMKR
KKPCDAGERTSANCANNETGNGKKVSVNNVGEAEPADTGFTGLTDNLRIGSFGNEVVIELRCAW
REGVLLEIMDVISDLHLDSHSVQSSTGDGLLCLTVNCKHKGSKIATPGMIKEALQRVAWIC
-Exemplary Arabidopsis thaliana Basic Helix Loop Helix domain TF
(Glabrous 3-Isoform 2) Nucleic Acid Coding Sequence
SEQ ID NO: 154
ATGGATGAAGAAACAATGGCTACCGGACAAAACAGAACAACTGTGCCAGAGAATCTGAAGAAAC
ACCTCGCAGTTTCAGTTCGAAACATTCAATGGAGTTATGGTATCTTTTGGTCTGTCTCTGCTTC
TCAGTCTGGAGTTTTAGAATGGGGAGATGGATACTATAATGGAGATATCAAAACGAGGAAGACG
ATTCAAGCTTCGGAGATCAAAGCTGATCAGCTTGGTCTACGGAGGAGCGAGCAGCTTAGCGAGC
TTTACGAGTCTCTCTCCGTCGCTGAATCTTCTTCTTCAGGCGTTGCTGCCGGATCTCAAGTCAC
CAGACGAGCTTCCGCCGCCGCACTTTCACCGGAAGATCTCGCCGACACCGAGTGGTACTATTTG
GTTTGTATGTCTTTCGTCTTCAACATTGGTGAAGGAATGCCTGGACGGACGTTTGCAAACGGTG
AACCGATATGGTTGTGCAACGCTCATACGGCGGATAGTAAAGTGTTTAGCCGTTCTCTTCTAGC
AAAAAGTGCTGCGGTTAAGACAGTGGTTTGCTTCCCGTTCCTTGGAGGAGTCGTTGAGATTGGT
ACCACAGAACATATTACGGAAGACATGAATGTAATACAATGCGTGAAGACATCATTCCTCGAAG
CCCCTGATCCGTACGCTACAATATTACCAGCAAGATCCGATTATCACATCGACAACGTTCTTGA
TCCGCAACAGATTCTAGGCGACGAGATTTACGCGCCTATGTTCAGTACGGAGCCTTTTCCAACA
GCTTCTCCGAGCAGAACTACCAACGGTTTCGATCAAGAACATGAACAAGTAGCAGATGATCATG
ATTCTTTCATGACCGAAAGAATCACTGGAGGAGCTTCTCAGGTGCAAAGCTGGCAGCTCATGGA
CGACGAGCTTAGTAACTGCGTTCACCAGTCGCTAAATTCCAGCGATTGCGTCTCTCAAACGTTT
GTTGAAGGGGCGGCTGGACGGGTTGCTTACGGTGCAAGAAAGAGTAGAGTTCAAAGACTAGGGC
AAATTCAAGAGCAACAGAGAAATGTGAAGACATTGTCATTTGATCCAAGAAACGACGACGTTCA
TTACCAAAGTGTGATCTCAACGATTTTTAAGACCAACCATCAGTTAATTCTCGGACCGCAGTTT
CGAAACTGCGATAAACAGTCAAGCTTCACTAGGTGGAAGAAATCATCGTCATCATCATCAGGAA
CCGCCACGGTCACGGCACCATCACAAGGAATGTTAAAGAAAATTATTTTCGATGTTCCGCGAGT
GCACCAGAAAGAGAAGTTAATGTTGGACTCACCAGAAGCCAGAGATGAAACTGGGAACCATGCG
GTTTTAGAGAAGAAGCGCCGCGAGAAATTGAACGAACGGTTCATGACCTTGAGAAAAATCATTC
CGTCAATCAACAAGATCGATAAAGTATCGATTCTTGACGATACGATAGAGTATCTTCAAGAACT
CGAGAGACGGGTTCAAGAACTAGAATCTTGCAGAGAATCAACCGATACAGAGACTCGTGGGACG
ATGACGATGAAGAGGAAGAAACCATGCGACGCAGGAGAAAGAACATCAGCTAATTGCGCAAATA
ATGAAACAGGAAATGGGAAGAAGGTGTCGGTTAACAATGTTGGTGAAGCCGAGCCAGCAGATAC
CGGTTTTACTGGTTTAACCGATAATTTAAGGATCGGTTCGTTTGGTAATGAGGTGGTTATTGAG
CTTAGATGTGCTTGGAGAGAAGGAGTATTGCTTGAGATAATGGATGTGATTAGTGATCTCCATT
TGGATTCTCATTCGGTTCAATCCTCGACCGGAGACGGTTTGCTCTGCTTAACCGTCAATTGCAA
GCACAAGGGGTCAAAAATAGCGACACCAGGAATGATCAAAGAAGCACTTCAAAGGGTTGCATGG
ATCTGTTGA
-Exemplary Arabidopsis thaliana Basic Helix Loop Helix domain TF
(Glabrous 3-Isoform 2) Amino Acid Sequence
SEQ ID NO: 155
MDEETMATGQNRTTVPENLKKHLAVSVRNIQWSYGIFWSVSASQSGVLEWGDGYYNGDIKTRKT
IQASEIKADQLGLRRSEQLSELYESLSVAESSSSGVAAGSQVTRRASAAALSPEDLADTEWYYL
VCMSFVFNIGEGMPGRTFANGEPIWLCNAHTADSKVFSRSLLAKSAAVKTVVCFPFLGGVVEIG
TTEHITEDMNVIQCVKTSFLEAPDPYATILPARSDYHIDNVLDPQQILGDEIYAPMFSTEPFPT
ASPSRTTNGFDQEHEQVADDHDSFMTERITGGASQVQSWQLMDDELSNCVHQSLNSSDCVSQTF
VEGAAGRVAYGARKSRVQRLGQIQEQQRNVKTLSFDPRNDDVHYQSVISTIFKINHQLILGPQF
RNCDKQSSFTRWKKSSSSSSGTATVTAPSQGMLKKIIFDVPRVHQKEKLMLDSPEARDETGNHA
VLEKKRREKLNERFMTLRKIIPSINKIDKVSILDDTIEYLQELERRVQELESCRESTDTETRGT
MTMKRKKPCDAGERTSANCANNETGNGKKVSVNNVGEAEPADTGFTGLIDNLRIGSFGNEVVIE
LRCAWREGVLLEIMDVISDLHLDSHSVQSSTGDGLLCLTVNCKHKGSKIATPGMIKEALQRVAW
IC
-Exemplary Arabidopsis thaliana Basic Helix Loop Helix domain TF
(Glabrous 3-Isoform 3) Nucleic Acid Coding Sequence
SEQ ID NO: 156
ATGGCTACCGGACAAAACAGAACAACTGTGCCAGAGAATCTGAAGAAACACCTCGCAGTTTCAG
TTCGAAACATTCAATGGAGTTATGGTATCTTTTGGTCTGTCTCTGCTTCTCAGTCTGGAGTTTT
AGAATGGGGAGATGGATACTATAATGGAGATATCAAAACGAGGAAGACGATTCAAGCTTCGGAG
ATCAAAGCTGATCAGCTTGGTCTACGGAGGAGCGAGCAGCTTAGCGAGCTTTACGAGTCTCTCT
CCGTCGCTGAATCTTCTTCTTCAGGCGTTGCTGCCGGATCTCAAGTCACCAGACGAGCTTCCGC
CGCCGCACTTTCACCGGAAGATCTCGCCGACACCGAGTGGTACTATTTGGTTTGTATGTCTTTC
GTCTTCAACATTGGTGAAGGAATGCCTGGACGGACGTTTGCAAACGGTGAACCGATATGGTTGT
GCAACGCTCATACGGCGGATAGTAAAGTGTTTAGCCGTTCTCTTCTAGCAAAAAGTGCTGCGGT
TAAGACAGTGGTTTGCTTCCCGTTCCTTGGAGGAGTCGTTGAGATTGGTACCACAGAACATATT
ACGGAAGACATGAATGTAATACAATGCGTGAAGACATCATTCCTCGAAGCCCCTGATCCGTACG
CTACAATATTACCAGCAAGATCCGATTATCACATCGACAACGTTCTTGATCCGCAACAGATTCT
AGGCGACGAGATTTACGCGCCTATGTTCAGTACGGAGCCTTTTCCAACAGCTTCTCCGAGCAGA
ACTACCAACGGTTTCGATCAAGAACATGAACAAGTAGCAGATGATCATGATTCTTTCATGACCG
AAAGAATCACTGGAGGAGCTTCTCAGGTGCAAAGCTGGCAGCTCATGGACGACGAGCTTAGTAA
CTGCGTTCACCAGTCGCTAAATTCCAGCGATTGCGTCTCTCAAACGTTTGTTGAAGGGGCGGCT
GGACGGGTTGCTTACGGTGCAAGAAAGAGTAGAGTTCAAAGACTAGGGCAAATTCAAGAGCAAC
AGAGAAATGTGAAGACATTGTCATTTGATCCAAGAAACGACGACGTTCATTACCAAAGTGTGAT
CTCAACGATTTTTAAGACCAACCATCAGTTAATTCTCGGACCGCAGTTTCGAAACTGCGATAAA
CAGTCAAGCTTCACTAGGTGGAAGAAATCATCGTCATCATCATCAGGAACCGCCACGGTCACGG
CACCATCACAAGGAATGTTAAAGAAAATTATTTTCGATGTTCCGCGAGTGCACCAGAAAGAGAA
GTTAATGTTGGACTCACCAGAAGCCAGAGATGAAACTGGGAACCATGCGGTTTTAGAGAAGAAG
CGCCGCGAGAAATTGAACGAACGGTTCATGACCTTGAGAAAAATCATTCCGTCAATCAACAAGA
TCGATAAAGTATCGATTCTTGACGATACGATAGAGTATCTTCAAGAACTCGAGAGACGGGTTCA
AGAACTAGAATCTTGCAGAGAATCAACCGATACAGAGACTCGTGGGACGATGACGATGAAGAGG
AAGAAACCATGCGACGCAGGAGAAAGAACATCAGCTAATTGCGCAAATAATGAAACAGGAAATG
GGAAGAAGGTGTCGGTTAACAATGTTGGTGAAGCCGAGCCAGCAGATACCGGTTTTACTGGTTT
AACCGATAATTTAAGGATCGGTTCGTTTGGTAATGAGGTGGTTATTGAGCTTAGATGTGCTTGG
AGAGAAGGAGTATTGCTTGAGATAATGGATGTGATTAGTGATCTCCATTTGGATTCTCATTCGG
TTCAATCCTCGACCGGAGACGGTTTGCTCTGCTTAACCGTCAATTGCAAGCACAAGGGGTCAAA
AATAGCGACACCAGGAATGATCAAAGAAGCACTTCAAAGGGTTGCATGGATCTGTTGA
-Exemplary Arabidopsis thaliana Basic Helix Loop Helix domain TF
(Glabrous 3-Isoform 3) Amino Acid Sequence
SEQ ID NO: 157
MATGQNRTTVPENLKKHLAVSVRNIQWSYGIFWSVSASQSGVLEWGDGYYNGDIKTRKTIQASE
IKADQLGLRRSEQLSELYESLSVAESSSSGVAAGSQVTRRASAAALSPEDLADTEWYYLVCMSF
VFNIGEGMPGRTFANGEPIWLCNAHTADSKVFSRSLLAKSAAVKTVVCFPFLGGVVEIGTTEHI
TEDMNVIQCVKTSFLEAPDPYATILPARSDYHIDNVLDPQQILGDEIYAPMFSTEPFPTASPSR
TTNGFDQEHEQVADDHDSFMTERITGGASQVQSWQLMDDELSNCVHQSLNSSDCVSQTFVEGAA
GRVAYGARKSRVQRLGQIQEQQRNVKTLSFDPRNDDVHYQSVISTIFKINHQLILGPQFRNCDK
QSSFTRWKKSSSSSSGTATVTAPSQGMLKKIIFDVPRVHQKEKLMLDSPEARDETGNHAVLEKK
RREKLNERFMTLRKIIPSINKIDKVSILDDTIEYLQELERRVQELESCRESTDTETRGTMTMKR
KKPCDAGERTSANCANNETGNGKKVSVNNVGEAEPADTGFTGLIDNLRIGSFGNEVVIELRCAW
REGVLLEIMDVISDLHLDSHSVQSSTGDGLLCLTVNCKHKGSKIATPGMIKEALQRVAWIC

### C2H2-Type Domain-Containing Protein (HAIR)

In some embodiments, a composition described herein comprises a transgenic C2H2 zing finger transcription factor encoding a HAIR protein. In some embodiments, a HAIR protein is encoded by the gene 104644359. In some embodiments, such a protein, among other things, may regulate trichome differentiation. In some embodiments, such a protein may heterodimerize with the transcription factor woolly.

In some embodiments, a HAIR protein encoding gene and/or transgene comprises a sequence encoding a peptide sequence that is at least 90%, 91%, 92%, 93%, 94%, 95%, 96%, 97%, 98%, 99%, or 100% identical to SEQ ID NO: 159 (or a portion thereof). In some embodiments, a HAIR protein encoding gene and/or transgene comprises a nucleotide sequence that is at least 90%, 91%, 92%, 93%, 94%, 95%, 96%, 97%, 98%, 99%, or 100% identical to SEQ ID NO:158 (or a portion thereof).

-Exemplary Solanum lycopersicum C2H2 zinc finger Transcription factor
(SL-Hair) Nucleic Acid Coding Sequence
SEQ ID NO: 158
ATGGAGAAGATTGGAAGAGAAGCTGTTGATTACATGAATATGAAGTCTTTCTCTCAACCCCTTA
GAAAAAAATCCATTAGACTTTTTGGTAAAGAATTTAGTGTTGGTGATAGTACTAACATGTCTGA
ATCAACTGATAAAAATCCTTTGCATCATGAACCTAAACCAAATACGATGAGTATCTCCGCGAAT
CGTATCGATAAAACAGGTCATGTTGATGAAATCAGCAGGAAATATGAATGTTACTATTGTTTTA
GGAGCTTTCCAACTTCTCAAGCTTTAGGAGGCCATCAAAATGCACACAAGAAAGAAAGACAAAA
TGCCAAACTATCTCATCTTCAGTCTTCAATAGTGCATGAGACGAACCGTAATAGATTTGGTGAA
CCATCCACTGCAGCTACAAGATTAACTCATTATCATTCAACATGGAGCAACATTAACAATAATA
ATGTTTATAGTCCTAATTACAATGAAGCATTTTGGCAAATTCCTCCAACAATTCATCATTATCA
GAATAATATTAATCCTCCATCTTCTTTTTCTCATGACTCATTTTTTCCTAATGATGAAGAGAAG
AGGGAAGTACAAAATCATGTGAGTTTAGATTTGCACTTATAA
-Exemplary Solanum lycopersicum C2H2 zinc finger Transcription factor
(SL-Hair) Amino Acid Sequence
SEQ ID NO: 159
MEKIGREAVDYMNMKSFSQPLRKKSIRLFGKEFSVGDSTNMSESTDKNPLHHEPKPNTMSISAN
RIDKTGHVDEISRKYECYYCFRSFPTSQALGGHQNAHKKERQNAKLSHLQSSIVHETNRNRFGE
PSTAATRLTHYHSTWSNINNNNVYSPNYNEAFWQIPPTIHHYQNNINPPSSFSHDSFFPNDEEK
REVQNHVSLDLHL

Modifying and/or Expressing Specific Transporter Channels

The present disclosure recognizes that in certain embodiments, formate uptake transmembrane transporters may be of particular usefulness for increasing indoor air quality. In some embodiments, formate uptake transmembrane transporters may facilitate active transport of formaldehyde. In some embodiments, formaldehyde uptake is mediated by formaldehyde specific transporters. In some embodiments, technologies described herein comprise transgenic expression of a formate transporter. In some embodiments, technologies described herein comprise transgenic expression of a formate transporter that has undergone directed evolution to increase specificity for formaldehyde. In some embodiments, technologies described herein comprise transgenic expression of a formaldehyde specific transporter.

The present disclosure recognizes that in certain embodiments, BTEX uptake transmembrane transporters may be of particular usefulness for increasing indoor air quality. In some embodiments, BTEX uptake transmembrane transporters may facilitate active transport of BTEX from an environment. In some embodiments, BTEX uptake is mediated by BTEX specific transporters. In some embodiments, technologies described herein comprise transgenic expression of a BTEX transporter. In some embodiments, technologies described herein comprise transgenic expression of a BTEX transporter that has undergone directed evolution to increase specificity for BTEX.

In some embodiments, compositions and methods of the present disclosure comprise modified (e.g., increased) levels of certain heterologous protein membrane transporters. In some embodiments, such a modification is facilitated through transgene introduction using materials and methods described herein.

### Oxalate: Formate Antiport Proteins

In some embodiments, a composition described herein comprises a transgenic Formate/oxalate Major Facilitator Family (MFS) antitransporter protein. In some embodiments, Formate/oxalate MFS antitransporter protein is encoded by the gene MFS. In some embodiments, such a protein, among other things, may participate in active transport of formate and/or formaldehyde.

In some embodiments, a Formate/oxalate MFS antitransporter protein encoding gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 161, 163, or 165 (or a portion thereof). In some embodiments, a Formate/oxalate MFS antitransporter protein encoding gene and/or transgene comprises a nucleotide sequence that is at least 90%, 91%, 92%, 93%, 94%, 95%, 96%, 97%, 98%, 99%, or 100% identical to any one of SEQ ID NOs: 160, 162, or 164 (or a portion thereof).

-Exemplary Oxalobacter formigenes Formate/oxalate MFS antiporter
(MFS of) Nucleic Acid Coding Sequence
SEQ ID NO: 160
ATGAATAATCCACAAACAGGACAATCAACAGGCCTCTTGGGCAATCGTTGGTTCTACTTGGTAT
TAGCAGTTTTGCTGATGTGTATGATCTCGGGTGTCCAATATTCCTGGACACTGTACGCTAACCC
GGTTAAAGACAACCTTGGCGTTTCTTTGGCTGCGGTTCAGACGGCTTTCACACTCTCTCAGGTC
ATTCAAGCTGGTTCTCAGCCTGGTGGTGGTTACTTCGTTGATAAATTCGGTCCAAGAATTCCAT
TGATGTTCGGTGGTGCGATGGTTCTCGCTGGCTGGACCTTCATGGGTATGGTTGACAGTGTTCC
TGCTCTGTATGCTCTTTATACTCTGGCCGGTGCAGGTGTTGGTATCGTTTACGGTATCGCGATG
AACACGGCTAACAGATGGTTCCCGGACAAACGCGGTCTGGCTTCCGGTTTCACCGCTGCCGGTT
ACGGTCTGGGTGTTCTGCCGTTCCTGCCACTGATCAGCTCCGTTCTGAAAGTTGAAGGTGTTGG
CGCAGCATTCATGTACACCGGTTTGATCATGGGTATCCTGATTATCCTGATCGCTTTCGTTATC
CGTTTCCCTGGCCAGCAAGGCGCCAAAAAACAAATCGTTGTTACCGACAAGGATTTCAATTCTG
GCGAAATGCTGAGAACACCACAATTCTGGGTTCTGTGGACCGCATTCTTTTCCGTTAACTTTGG
TGGTTTGCTGCTGGTTGCCAACAGCGTCCCTTACGGTCGCAGCCTCGGTCTTGCCGCAGGTGTG
CTGACGATCGGTGTTTCGATCCAGAACCTGTTCAATGGTGGTTGCCGTCCTTTCTGGGGTTTCG
TTTCCGATAAAATCGGCCGTTACAAAACCATGTCCGTCGTTTTCGGTATCAATGCTGTTGTTCT
CGCACTTTTCCCGACGATTGCTGCCTTGGGCGATGTAGCCTTTATCGCCATGTTGGCAATCGCA
TTCTTCACATGGGGTGGTAGCTACGCTCTGTTCCCATCGACCAACAGCGATATTTTCGGTACGG
CATACTCTGCCAGAAACTATGGTTTCTTCTGGGCTGCAAAAGCAACTGCCTCGATCTTCGGTGG
TGGTCTGGGTGCTGCAATTGCAACCAACTTCGGATGGAATACCGCTTTCCTGATTACTGCGATT
ACTTCTTTCATCGCATTTGCTCTGGCTACCTTCGTTATTCCAAGAATGGGCCGTCCAGTCAAGA
AAATGGTCAAATTGTCTCCAGAAGAAAAAGCTGTACATTAA
-Exemplary Oxalobacter formigenes Formate/oxalate MFS antiporter
(MFS of) Amino Acid Sequence
SEQ ID NO: 161
MNNPQTGQSTGLLGNRWFYLVLAVLLMCMISGVQYSWTLYANPVKDNLGVSLAAVQTAFTLSQV
IQAGSQPGGGYFVDKFGPRIPLMFGGAMVLAGWTFMGMVDSVPALYALYTLAGAGVGIVYGIAM
NTANRWFPDKRGLASGFTAAGYGLGVLPFLPLISSVLKVEGVGAAFMYTGLIMGILIILIAFVI
RFPGQQGAKKQIVVTDKDFNSGEMLRTPQFWVLWTAFFSVNFGGLLLVANSVPYGRSLGLAAGV
LTIGVSIQNLFNGGCRPFWGFVSDKIGRYKTMSVVFGINAVVLALFPTIAALGDVAFIAMLAIA
FFTWGGSYALFPSTNSDIFGTAYSARNYGFFWAAKATASIFGGGLGAAIATNFGWNTAFLITAI
TSFIAFALATFVIPRMGRPVKKMVKLSPEEKAVH
-Exemplary Methylobacterium sp. Formate/oxalate MFS antiporter (MFS
mb1) Nucleic Acid Coding Sequence
SEQ ID NO: 162
ATGGAACGCCAGGATTCGCCGTCGGCGAAATGGTGGCAGCTCGCCTTCGGCGTGATCTGCATGG
CCATGATCGCCAACCTCCAATACGGTTGGACGTTGTTCGTGGACCCGATCGACCAGCGCTACCA
CTGGGGACGCGCGGCGATCCAGCTCGCCTTCACGCTGTTCGTCGCCACCGAGACCTGGCTGGTC
CCGGTCGAGGCGTGGTTCGTCGACCGCTACGGCCCGAAGATCGTGGTCGCGTTCGGCGGCGTGA
TGATCGCCCTCGCCTGGACGATCAACGCCTACGCCGACAGCCTGGCGATGCTCTATCTCGGCGC
CGTCATCGCCGGCATCGGTGCGGGCTCGGTCTACGGCACCTGCGTGGGCAACGCGCTCAAGTGG
TTCCCGCATCGCCGCGGCCTCGCCGCCGGTGCCACCGCGGCCGGCTTCGGCGCGGGTGCCGCCA
TCACGGTGGTACCGATCGCCCGCATGATCGCGTCGAGCGGTTACCAGGACGCCTTCCTGTATTT
CGGCATCGGTCAGGGCGCCGTGGTCCTCGCGCTCGCCTTCCTGCTGCGCAAGCCGTCGACCAAC
TCGCCGGTCCAGCGCAAGAGCACCCGCCTGCCGCAGACCAAGGTCGACCGCAGCCCCCGCGAGG
CGGTGCGCACCCCGGTCTTCTGGGTGATGTACGCCATGTTCGTGATGGTCGCCTCCGGCGGCCT
GATGGCGGCGGCGCAGATCGCCCCGATCGCCCACGACTTCCAGGTGGCGGGCGTGCCGGTGAGC
CTGTTCGGCCTCCAGATGGCGGCGCTGACGCTTGCGATCTCGCTCGACCGGATCTTCGACGGGT
TCGGGCGGCCGTTCTTCGGCTACGTCTCCGACAACATCGGCCGCGAGAACACGATGTTCATCGC
CTTCTCGACGGCGGCGCTGGCGGTGATCGTGCTGCTGACCTACGGTCACATCCCGATGGTCTTC
GTGCTGGCCACCGCGGTGTATTTCGGGGTGTTCGGCGAGATCTACTCGCTGTTCCCGGCGACCT
GCGGCGACACGTTCGGCTCCAAGTACGCCGCCAGCAATGCCGGCCTGCTCTACACCGCCAAGGG
CACCGCGGCGTTCCTCGTGCCCTTCGCCAGCCTCCTGTCGGCGGCCTACGGCTGGTCGGCGGTG
TTCACGCTGATCATCGTGCTCAACGTGACGGCGGCGGCGATGGCGATGTTCGTCCTGCGCCCGA
TGCGGGCCCGCTACCTCGCCGCGGAGGAGCATCCCGCGGCGCTCAGCGCCCATCCGATCTAA
-Exemplary Methylobacterium sp. Formate/oxalate MFS antiporter (MFS
mb1) Amino Acid Sequence
SEQ ID NO: 163
MERQDSPSAKWWQLAFGVICMAMIANLQYGWTLFVDPIDQRYHWGRAAIQLAFTLFVATETWLV
PVEAWFVDRYGPKIVVAFGGVMIALAWTINAYADSLAMLYLGAVIAGIGAGSVYGTCVGNALKW
FPHRRGLAAGATAAGFGAGAAITVVPIARMIASSGYQDAFLYFGIGQGAVVLALAFLLRKPSTN
SPVQRKSTRLPQTKVDRSPREAVRTPVFWVMYAMFVMVASGGLMAAAQIAPIAHDFQVAGVPVS
LFGLQMAALTLAISLDRIFDGFGRPFFGYVSDNIGRENTMFIAFSTAALAVIVLLTYGHIPMVF
VLATAVYFGVFGEIYSLFPATCGDTFGSKYAASNAGLLYTAKGTAAFLVPFASLLSAAYGWSAV
FTLIIVLNVTAAAMAMFVLRPMRARYLAAEEHPAALSAHPIRAA
-Exemplary Methylobacterium sp. Formate/oxalate MFS antiporter (MFS
mb2) Nucleic Acid Coding Sequence
SEQ ID NO: 164
ATGTCCGAGATCGTCAAACCGGCGGGGCGTGGCCGATGGCTGCAACTCGCCTTCGGCGTGGTCT
GCATGTGCATGATCGCCAACATGCAGTACGGTTGGACCTTCTTCGTGAACCCGATGCAGGAGCG
GCACGGCTGGGATCGCGCGGCGATCCAGGTGGCGTTCACGCTGTTCGTCGTCACCGAGACGTGG
CTGGTCCCGATCGAGGGCTGGTTTGTCGACAAGTATGGCCCGCGGATCGTCACGCTGTTCGGCG
GCCTGCTCTGCGGCATCGCCTGGGTGATCAACTCCTACGCCGACTCGCTCACCGTCCTGTACAT
CGCGGCCGCGATCGGCGGCACCGGCGCCGGTGCGGTCTACGGAACCTGCGTCGGCAATTCGCTG
AAGTGGTTTCCCGACCGACGCGGCCTCGCCGCGGGCATCACCGCGATGGGCTTCGGCGCGGGCT
CGGCCCTGACCGTCGTGCCGATCCAGGCCATGATCAAGTCGCAGGGCTACGAGGCGGCGTTCTT
CTACTTCGGTATCGGGCAGGGCGTCATCGTGATGCTCATCGCCCTGTTCCTGCGGTCGCCCGCG
AAGGGGCAGGTTCCGGAGATCGCCCGGGTCAGCCAGTCGAAGCGCGACTACAAGCCCTCCGAGA
TGGTCCGCACGCCGATCTTCTGGGTCATGTACGCGATGTTCGTCATGATGGCGGCCGGCGGCCT
GATGGCGACCGCGCAGCTCGGCCCGATCGCCAAGGACTTCAAGATCGCCGACGTTCCGGTCTCG
CTGCTCGGGATCACGCTGCCGGCGCTGACCTTCGCGGCCACGCTCGACCGGGTGCTCAACGGCG
TGACGCGTCCGTTCTTCGGCTGGGTCTCCGACCATATCGGCCGCGAGAACACGATGTTCCTGTC
CTTCGCGATCGAAGGCCTGGGCATCTACGCGCTCAGCCAGTTCGGCCAGAACCCGATCGCCTTC
GTGCTTCTGACCGGTCTCGTGTTCTTTGCCTGGGGTGAGATCTACTCCCTGTTCCCGGCGACCT
GCGGAGACACGTTCGGCTCGAAATACGCCGCCACCAATGCCGGTCTGCTCTATACGGCCAAGGG
CACGGCGGCGCTGATCGTCCCCTATACCAGCGTGCTCACGACCATGACCGGGAGCTGGCACGCG
GTGTTCCTGGCGGCAGCGGCCCTCAACATCGTCGCGGCTCTGCTGGCGCTCTTCGTCCTGAAGC
CGATGCGGGCCGCCTATACCAAGAAGCGCGAAGCGAGCCTCGCGCCGGTCCTGGCCCAGTAA
-Exemplary Methylobacterium sp. Formate/oxalate MFS antiporter (MFS
mb2) Amino Acid Sequence
SEQ ID NO: 165
MSEIVKPAGRGRWLQLAFGVVCMCMIANMQYGWTFFVNPMQERHGWDRAAIQVAFTLFVVTETW
LVPIEGWFVDKYGPRIVTLFGGLLCGIAWVINSYADSLTVLYIAAAIGGTGAGAVYGTCVGNSL
KWFPDRRGLAAGITAMGFGAGSALTVVPIQAMIKSQGYEAAFFYFGIGQGVIVMLIALFLRSPA
KGQVPEIARVSQSKRDYKPSEMVRTPIFWVMYAMFVMMAAGGLMATAQLGPIAKDFKIADVPVS
LLGITLPALTFAATLDRVLNGVTRPFFGWVSDHIGRENTMFLSFAIEGLGIYALSQFGQNPIAF
VLLTGLVFFAWGEIYSLFPATCGDTFGSKYAATNAGLLYTAKGTAALIVPYTSVLTTMTGSWHA
VFLAAAALNIVAALLALFVLKPMRAAYTKKREASLAPVLAQ

### FADL Membrane Channel Proteins

In some embodiments, a composition described herein comprises a transgenic FADL membrane channel protein. In some embodiments, a FADL membrane channel protein is encoded by the gene Tod X. In some embodiments, a FADL membrane channel protein is encoded by the gene Cym D. In some embodiments, a FADL membrane channel protein is a member of the Porine superfamily. In some embodiments, such a protein, among other things, may participate in active transport of BTEX.

In some embodiments, a FADL membrane channel protein encoding gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 167 or 169 (or a portion thereof). In some embodiments, a FADL membrane channel protein encoding gene and/or transgene comprises a nucleotide sequence that is at least 90%, 91%, 92%, 93%, 94%, 95%, 96%, 97%, 98%, 99%, or 100% identical to any one of SEQ ID NOs: 166 or 168 (or a portion thereof).

-Exemplary Pseudomonas putida FADL membrane channel protein (Tod X)
Nucleic Acid Coding Sequence
SEQ ID NO: 166
ATGAAGATTGCCAGCGTGCTCGCACTGCCTTTGAGTGGATATGCTTTCAGTGTGCATGCTACAC
AGGTTTTCGACCTGGAAGGTTATGGAGCGATCTCTCGTGCCATGGGTGGCACCAGTTCATCGTA
TTATACCGGTAATGCTGCGCTGATTAGTAATCCCGCTACATTGAGTTTTGCTCCGGACGGAAAT
CAGTTTGAGCTCGGGCTGGACGTGGTGACTACCGATATCAAGGTTCACGACAGCCACGGAGCAG
AGGCAAAAAGCAGCACGAGATCCAATAATCGAGGCCCCTATGTGGGTCCACAATTGAGCTATGT
TGCTCAGTTGGATGACTGGCGTTTCGGTGCTGGATTGTTTGTCAGTAGCGGGTTGGGTACAGAG
TATGGAAGTAAAAGTTTTCTATCACAGACAGAAAACGGAATCCAGACCAGCTTTGATAATTCCA
GCCGTCTGATCGTATTGCGCGCTCCTATTGGCTTTAGTTATCAAGCCACATCAAAGCTCACCTT
CGGCGCTAGTGTCGATCTGGTCTGGACTTCACTCAACCTTGAACTTCTACTTCCATCATCTCAG
GTGGGAGCCCTGACTGCGCAGGGGAATCTTTCAGGCGGTTTAGTTCCCTCGCTGGCTGGATTCG
TCGGGACAGGTGGTGCCGCCCATTTCAGTCTAAGTCGCAACAGTACCGCTGGTGGCGCCGTGGA
TGCGGTCGGTTGGGGCGGGCGCTTGGGACTTACCTACAAACTCACGGATAACACTGTCCTAGGT
GCGATGTACAACTTCAAGACTTCGGTGGGCGATCTCGAGGGGAAGGCGACACTTTCTGCTATCA
GTGGTGATGGAGCGGTGCTTCCATTGGATGGCGATATCCGTGTAAAAAACTTTGAGATGCCCGC
CAGTCTGACGCTTGGCCTCGCTCATCAGTTCAATGAGCGTTGGGTAGTTGCTGCTGATATCAAG
CGTGCCTACTGGGGTGATGTAATGGATAGCATGAATGTGGCTTTCATCTCGCAGTTGGGCGGGA
TCGATGTCGCATTGCCACACCGCTATCAGGATATAACGGTGGCCTCAATCGGCACTGCTTACAA
ATATAACAATGATTTAACGCTTCGTGCTGGATATAGCTATGCACAACAGGCGCTAGACAGCGAA
CTGATATTGCCAGTGATTCCTGCTTATTTGAAGCGGCACGTTACTTTCGGTGGCGAGTATGACT
TTGACAAGGACTCCAGGATCAATTTGGCAATTTCTTTTGGCCTGAGAGAGCGCGTGCAGACGCC
ATCGTACTTGGCAGGCACCGAGATGTTGCGGCAAAGCCACAGTCAAATAAATGCAGTGGTTTCC
TATAGCAAAAATTTTTAA
-Exemplary Pseudomonas putida FADL membrane channel protein (Tod X)
Amino Acid Sequence
SEQ ID NO: 167
MKIASVLALPLSGYAFSVHATQVEDLEGYGAISRAMGGTSSSYYTGNAALISNPATLSFAPDGN
QFELGLDVVTTDIKVHDSHGAEAKSSTRSNNRGPYVGPQLSYVAQLDDWRFGAGLFVSSGLGTE
YGSKSFLSQTENGIQTSFDNSSRLIVLRAPIGFSYQATSKLTFGASVDLVWTSLNLELLLPSSQ
VGALTAQGNLSGGLVPSLAGFVGTGGAAHFSLSRNSTAGGAVDAVGWGGRLGLTYKLTDNTVLG
AMYNFKTSVGDLEGKATLSAISGDGAVLPLDGDIRVKNFEMPASLTLGLAHQFNERWVVAADIK
RAYWGDVMDSMNVAFISQLGGIDVALPHRYQDITVASIGTAYKYNNDLTLRAGYSYAQQALDSE
LILPVIPAYLKRHVTFGGEYDFDKDSRINLAISFGLRERVQTPSYLAGTEMLRQSHSQINAVVS
YSKNF
-Exemplary Pseudomonas putida FADL membrane channel protein (Cym D)
Nucleic Acid Coding Sequence
SEQ ID NO: 168
ATGAAAAAAACAATATACAGCTTAAGTGCCTGCGGCATTTTGACGTGCTTGTACTGTGGTATTG
CGTCTGCAACAGATGCTTTCAACCTCGTCGGGGTTGGACCGGTTTCCCAAGGTATGGGGGGGAT
TGGTGCAGCCTTCAATATCGGGGCACAAGGTATGATGCTGAACCCGGCAACGCTTACTCAGATG
CAAGAAGGTATGCATCTGGGGCTGGGAATGGACATCATTACTGCGGAATTGGAAGTCAAGAATA
CCGCTACCGGCGAAAAAGCCGACTCCCATAGTCGTGGGCGCAACAACGGGCCTTACGTGGCGCC
TGAGCTTTCTTTGGTGTGGCGTGGTGAGCGATATGCGCTGGGAGTCGGTGCTTTTGCTTCCGAT
GGGGTTGGAACCCAGTTTGGAGACACCAGCTTTCTCTCGCGTACCACGACCAATAATCTTAATA
CAGGGCTGGAAAACTACTCCCGTCTGATAGTTTTGCGGATACCGTTCTCTGCGGCTTACCAGGT
GAACGAGAAGTTGTCCGTCGGGGCATCGTTGGATGCTGTGTGGACGTCGGTGAACTTGGGACTC
CTACTGGATACCACACAGATTGGTACATTGGTTGGACAAGGCCAGGTGTCCGGCTCATTGATGC
CAGCGTTGCTGAGCGTGCCGGAGCTGTCGGCAGGTTATCTATCCGCGGACAATCACCGTGCCAG
CGGTGGTGGCGTGGACTCCTGGGGCATAGGTGGCCGGCTTGGTCTGACCTATCAGTTGACCCCA
AAAACACGGGTGGGGATTGTATACAACTTCAAGACCCATGTTGGAGACCTGTCTGGCAATGCCG
ATTTGACGGCAGTAAGCGCTGTCGCGGGTAATATCCCTCTCTCGGGTGAACTCAAGCTACATAA
CTTCGAGATGCCAGCATCTCTCGTTGCGGGCATCAGTCACGAATTCAGTGATCAGTTTGCTGTT
GCGTTCGACTACAAGCGTGTCTACTGGAGCGATGTCATGGATGACATAGAAGTCAACTTCAAGC
AGAAAGCCACGGGCGACACTATCAATCTGAAACTGCCTTTCAATTATCGGGACACCAACGTGTA
TTCGTTGGGAGCGCAATACCGCTACGGTGCGAACTGGGTGTTTCGAGCGGGCGTGCACTATGCC
CAACTGGCCAACCCTTCAAGTGGTACAATGCCAATCATTCCTTCGACACCGACTACCAGTCTCT
CGGGAGGCTTTTCATATGCCTTCAGCCCTGAGGATGTAGTCGATTTTTCTCTGGCCTACGGATT
CAAGAAGAAAGTATCCAATGACAGCCTGCCGATCACCGACAAGCCCATCGAAGTATCGCATTCG
CAGATAGTTACATCGATTTCCTATACCAAGAGTTTCTAG
-Exemplary Pseudomonas putida FADL membrane channel protein (Cym D)
Amino Acid Sequence
SEQ ID NO: 169
MKKTIYSLSACGILTCLYCGIASATDAFNLVGVGPVSQGMGGIGAAFNIGAQGMMLNPATLTQM
QEGMHLGLGMDIITAELEVKNTATGEKADSHSRGRNNGPYVAPELSLVWRGERYALGVGAFASD
GVGTQFGDTSFLSRTTINNLNTGLENYSRLIVLRIPFSAAYQVNEKLSVGASLDAVWTSVNLGL
LLDTTQIGTLVGQGQVSGSLMPALLSVPELSAGYLSADNHRASGGGVDSWGIGGRLGLTYQLTP
KTRVGIVYNFKTHVGDLSGNADLTAVSAVAGNIPLSGELKLHNFEMPASLVAGISHEFSDQFAV
AFDYKRVYWSDVMDDIEVNFKQKATGDTINLKLPFNYRDTNVYSLGAQYRYGANWVFRAGVHYA
QLANPSSGTMPIIPSTPTTSLSGGFSYAFSPEDVVDFSLAYGFKKKVSNDSLPITDKPIEVSHS
QIVTSISYTKSF

### Modifying Metabolic Pathways

Among other things, the present disclosure provides compositions, methods of producing, and methods of using genetically modified plants with optimized metabolic pathways capable of providing useful catabolic and/or anabolic functions.

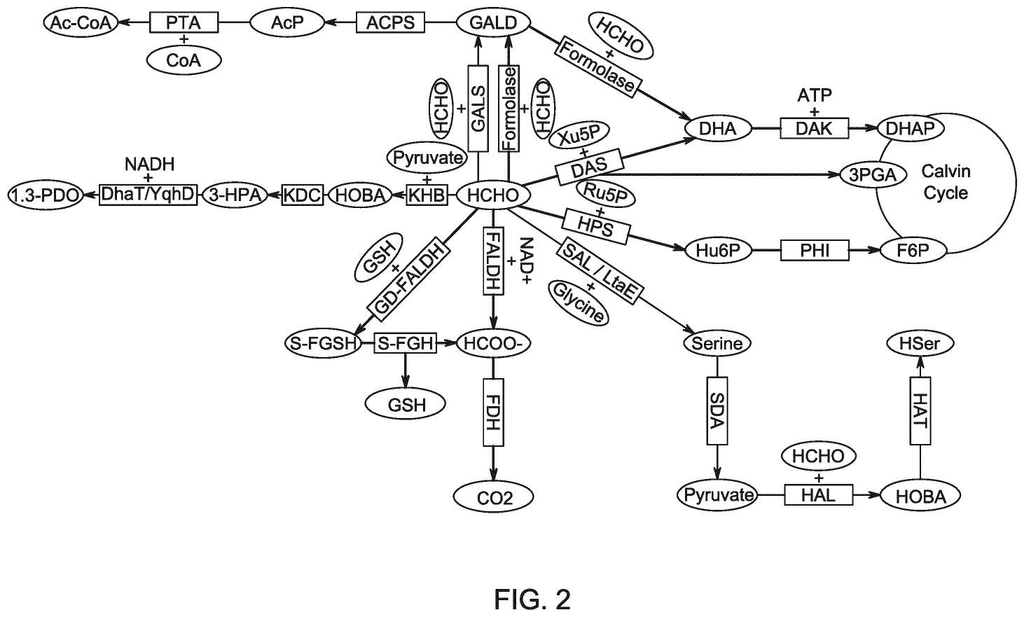

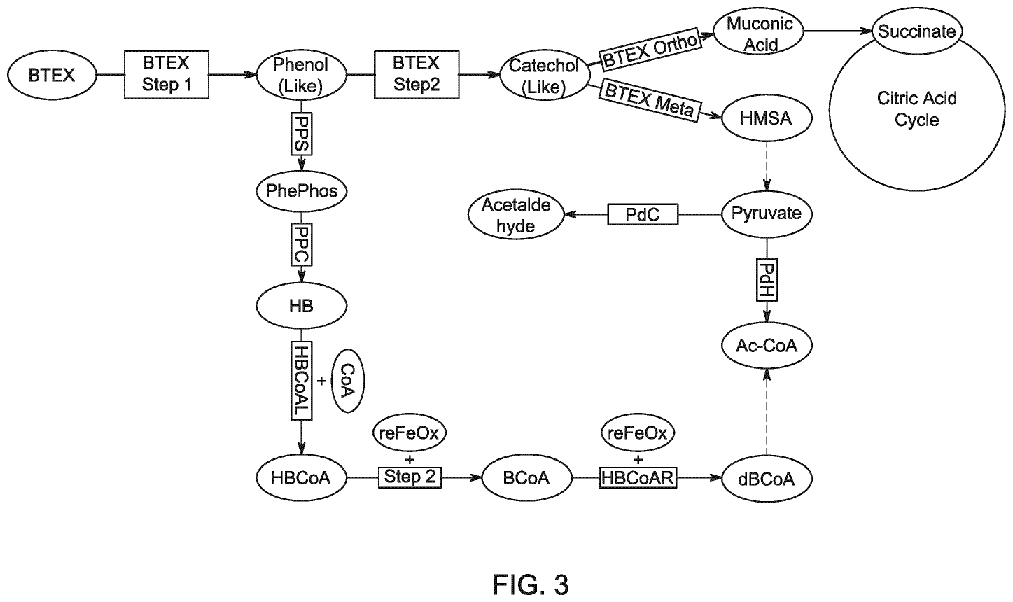

In certain embodiments, once inside an engineered plant (e.g., root, leaf, stem, etc.), VOCs can be metabolized, and undergo degradation, storage, and/or excretion. For example, in certain embodiments, formaldehyde can be transformed into molecules that can serve as a carbon source and be used for biosynthesis of novel molecules, and after transformation to CO₂ the carbon may also be incorporated into the plant material via the Calvin cycle. In some embodiments, an engineered plant comprises an engineered pathway as described in FIG. 2. In some embodiments, an engineered plant comprises an engineered pathway as described in FIG. 3.

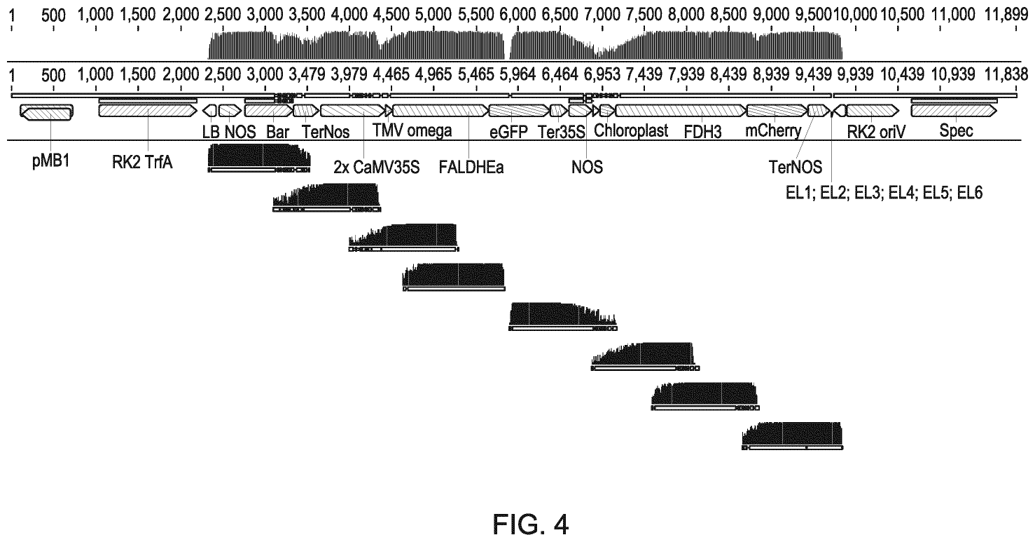

In certain embodiments, a targeted VOC is formaldehyde (HCHO), which may act as a carbon source entering the Calvin-Benson Cycle. In some embodiments of such a metabolic pathway, HCHO may be metabolized through the following metabolic mechanism (pathway 1): 1) Dihydroxyacetone synthase (DAS) combining HCHO and xylulose 5-phosphate (Xu5P) producing Glyceraldehyde 3-phosphate (3PGA) in turn entering into the Calvin-Benson Cycle, and dihydroxyacetone (DHA) 2) Dihydroxyacetone Kinase (DAK) phosphorylating DHA into Dihydroxyacetone phosphate (DHAP); 3) DHAP entering into the endogenous plant Calvin-Benson Cycle. In certain embodiments, one or more of the enzymatic components of this pathway may be introduced as a transgene as described herein (see FIGS. 4-9).

In certain embodiments, a targeted VOC is formaldehyde (HCHO), which may act as a carbon source entering the Calvin-Benson Cycle. In some embodiments of such a metabolic pathway, HCHO may be metabolized through the following metabolic mechanism (pathway 2): 1) 3-Hexulose-6-phosphate synthase (HPS) combining HCHO and ribulose 5-phosphate (Ru5P) producing D-arabino-3-hexulose 6-phosphate (Hu6P) 2) 6-phospho-3-hexuloisomerase (PHI) isomerizing Hu6P into fructose 6-phosphate (F6P); 3) F6P entering into the endogenous plant Calvin-Benson Cycle. In certain embodiments, one or more of the enzymatic components of this pathway may be introduced as a transgene as described herein (see FIGS. 4-9).

In certain embodiments, a targeted VOC is formaldehyde (HCHO), which may act as a carbon source entering the plant endogenous metabolism. In some embodiments of such a metabolic pathway, HCHO may be metabolized through the following metabolic mechanism (pathway 3): 1) Glutathione-independent formaldehyde dehydrogenase (FALDH) and/or Glutathione-dependent formaldehyde dehydrogenase (GSH-FALDH) with cofactor NAD+ producing Formate; 2) Formate dehydrogenase (FDH) with cofactor NAD+ producing CO2; 3) Entry of CO2 into any plant endogenous metabolism pathways, like the Calvin-Benson Cycle. In certain embodiments, Serine hydroxymethyltransferase 1, mitochondrial (SHM1) and/or(S)-2-hydroxy-acid oxidase (GLO1 and/or GLO2) may also impact the metabolic flux of HCHO metabolism as described herein, for example, through the production of L-Serine and/or oxocarboxylate. In certain embodiments, one or more of the enzymatic components of this pathway may be introduced as a transgene as described herein (see FIGS. 4-9).

In certain embodiments, a targeted VOC is formaldehyde (HCHO), which may act as a carbon source entering the Calvin-Benson Cycle. In some embodiments of such a metabolic pathway, HCHO may be metabolized through the following metabolic mechanism (pathway 4): 1) Formolase (FLS) converting two molecules of HCHO into glycolaldehyde (GALD) 2) Formolase combining a molecule of GALD and a molecule of HCHO into dihydroxyacetone (DHA) 3) Dihydroxyacetone Kinase (DAK) phosphorylating DHA into Dihydroxyacetone phosphate (DHAP); 3) DHAP entering into the endogenous plant Calvin-Benson Cycle. In certain embodiments, one or more of the enzymatic components of this pathway may be introduced as a transgene as described herein (see FIGS. 4-9).

In certain embodiments, a targeted VOC is formaldehyde (HCHO), which may act as a carbon source used to synthesize acetyl coenzyme A (Ac-CoA). In some embodiments of such a metabolic pathway, HCHO may be metabolized through the following metabolic mechanism (pathway 5): 1) glycolaldehyde synthase (GALS) converting two molecules of HCHO into glycolaldehyde (GALD) 2) acetyl-phosphate synthase (ACPS) adding inorganic phosphate (Pi) to GALD to produce acetyl-phosphate (AcP) 3) phosphate acetyltransferase (PTA) combines coenzyme A with AcP to produce acetyl coenzyme A (Ac-COA) 4) Ac-COA entering into various endogenous plant metabolic pathways, for example fatty acid synthesis. In certain embodiments, one or more of the enzymatic components of this pathway may be introduced as a transgene as described herein (see FIGS. 4-9).

In certain embodiments, a targeted VOC is formaldehyde (HCHO), which may act as a carbon source used to synthesize 1,3-Propanediol. In some embodiments of such a metabolic pathway, HCHO may be metabolized through the following metabolic mechanism (pathway 6): 1) 2-keto-4-hydroxybutyrate aldolase (KHB) combines HOCH with pyruvate to form 4-hydroxy-2-oxobutanoate (2-keto-4-hydroxybutyrate) 2) branched-chain alpha-keto acid decarboxylase (KDC) or pyruvate decarboxylase (PDC) combining 4-hydroxy-2-oxobutanoate with CO2 to form 3-Hydroxypropionaldehyde (Reuterine) 3) NADH-dependent 1,3-PDO oxidoreductase (DhaT) or a non-specific NADPH-dependent alcohol dehydrogenase (YqhD) turns reuterine into 1,3-Propanediol 4) 1,3-Propanediol integrating various endogenous plant metabolic pathways. In certain embodiments, one or more of the enzymatic components of this pathway may be introduced as a transgene as described herein (see FIGS. 4-9).

In certain embodiments, a targeted VOC is formaldehyde (HCHO), which may act as a carbon source used to synthesize homoserine. In some embodiments of such a metabolic pathway, HCHO may be metabolized through the following metabolic mechanism (pathway 7): 1) serine aldolase (SAL) or threonine aldolase (LtaE) combining HOCH with glycine to form serine 2) serine being then deaminated to pyruvate by serine deaminase (SDA) 3) 4-hydroxy-2-oxobutanoate (HOB) aldolase (HAL) combining formaldehyde and pyruvate to from HOB 4) HOB aminotransferase (HAT) turning HOB into Homoserine 5) Homoserine (HSer) integrating various endogenous plant metabolic pathways. In certain embodiments, one or more of the enzymatic components of this pathway may be introduced as a transgene as described herein (see FIGS. 4-9.

In certain embodiments, a targeted VOC is benzene, toluene, ethylbenzene, and/or xylene (BTEX), any of which may act as a carbon source. In such a metabolic pathway, BTEX may be metabolized in the following mechanism (pathway 8): 1) A monooxygenase or hydrolase adds on or two-OH group to the benzene ring, turning it into a phenolic compound. These enzymes are here referred to as “BTEX Step 1” and can be: cytochrome P450 monooxygenase (P450-RR) Toluene, O-xylene Monooxygenase Oxygenase Subunit alpha (TouA-P-OX), benzene monooxygenase oxygenase subunit (BmoA-Pa) Toluene-4-monooxygenase (TmoF_Pm) Toluene monooxygenase alpha subunit (TbuA1-Mp), aromatic ring-hydroxylating dioxygenase subunit alpha (TodC1 (bnzA)_Pp), hydroxylase alpha subunit (tmoA_P_sp_BDa59), hydroxylase alpha subunit (tmoA_Pm), Eng-Phenylalanine Hydroxylase (PHOH-Pt) 2) A monooxygenase or hydrolase might add a second-OH group to the benzene ring of the phenolic compound, turning it into a catechol-like compound. These enzymes are here referred to as “BTEX Step 2” and can be: phenol hydroxylase component phP (PH_PS_OX1) Phenol monooxygenase (PMO-cc) Phenol hydroxylase (PH-CC or PH-AO). 2) A dioxygenase cuts open the benzene ring of the catecholic compound, turning it either into cis, cis-Muconate or 2-Hydroxymuconate semialdehyde. These enzymes are here referred to respectively as “BTEX Ortho” and “BTEX Meta” and can be: 3-isopropylcatechol-2,3-dioxygenase (lpbc_P_sp_JR1), LE2_PSEPU Metapyrocatechase (xylE_Pp), extradiol dioxygenase (Dbtc_B_DBT1_OX), catechol 2,3-dioxygenase (tbuE_Rp C) Chlorocatechol 1,2-dioxygenase (tfdc), catA_Pp, catA_Pr, salD_Pr. In certain embodiments, one or more of the enzymatic components of this pathway may be introduced as a transgene as described herein (see FIGS. 4-9).

### Formaldehyde Metabolism

In some embodiments, the present disclosure provides compositions and methods for engineering plants to be effective metabolizers of formaldehyde. In certain embodiments, one or more constructs and/or transgenes described herein are engineered into a plant to facilitate metabolism of formaldehyde. In some embodiments, a pathway that is engineered is described in FIG. 2.

### A) Ribulose Monophosphate Pathway.

In some embodiments, compositions and methods described herein comprise introduction of one or more genes coding for one or more enzymes such as: 3-hexulose-6-phosphate synthase (HPS) and 6-phospho-3-hexuloisomerase (PHI). In some embodiments, these enzymes metabolize the substrates Ru5P and HCHO to produce Hu6P and/or F6P. In some embodiments, Hu6P and/or F6P function as components of the Calvin-Benson cycle, a photosynthetic carbon fixation pathway. In some embodiments, HPS and PHI function are incorporated into one enzyme, and only one gene is introduced that facilitates the conversion of formaldehyde directly to fructose 6-phosphate.

### 3-Hexulose-6-Phosphate Formaldehyde Lyase (HPS/PHI)

In some embodiments, a composition described herein comprises a transgenic HPS/PHI protein. In some embodiments, such a protein, among other things, may utilize formaldehyde as a substrate and produce fructose 6-phosphate (F6P).

In some embodiments, a HPS/PHI gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 171 or 173 (or a portion thereof). In some embodiments, a HPS/PHI gene and/or transgene comprises a nucleotide sequence that is at least 90%, 91%, 92%, 93%, 94%, 95%, 96%, 97%, 98%, 99%, or 100% identical to any one of SEQ ID NOs: 170 or 172 (or a portion thereof).

-Exemplary Pyrococcus horikoshii OT3 3-hexulose-6-phosphate
formaldehyde lyase (HPS/PHI-archea) Nucleic Acid Coding Sequence
SEQ ID NO: 170
ATGATCCTTCAGGTTGCTTTGGATCTAACGGACATCGAACAGGCTATATCAATAGCAGAGAAAG
CAGCCAGGGGTGGCGCGCATTGGCTTGAGGTTGGAACTCCGCTAATCAAGAAGGAAGGTATGCG
TGCGGTCGAGTTATTGAAAAGACGTTTCCCTGACAGGAAGATTGTTGCAGATCTCAAAACCATG
GACACCGGGGCGCTTGAAGTTGAGATGGCCGCTAGACACGGGGCGGACGTCGTTTCGATTTTGG
GCGTTGCTGATGATAAGACCATCAAGGACGCTTTAGCAGTTGCCAGGAAATACGGTGTGAAAAT
CATGGTGGATTTGATCGGAGTAAAAGACAAGGTGCAGAGAGCAAAAGAGTTAGAACAAATGGGA
GTTCATTACATACTTGTACATACGGGAATCGACGAACAAGCACAGGGGAAAACTCCTCTTGAAG
ATCTAGAGAAGGTGGTCAAGGCCGTAAAGATTCCAGTGGCAGTGGCCGGTGGATTAAATCTGGA
AACAATCCCCAAGGTTATAGAACTCGGCGCGACTATAGTGATTGTGGGCAGTGCAATCACTAAG
AGCAAAGACCCAGAGGGAGTGACGAGGAAGATTATCGACTTATTTTGGGATGAGTACATGAAAA
CGATCCGAAAAGCGATGAAGGATATAACTGATCACATAAACGAAGTTGCAGACAAGCTCAGACT
CGACGAGGTGAGAGGTCTAGTGGATGCAATGATAGGCGCAAATAAAATCTTCATCTACGGCGCC
GGTCGGTCTGGCCTTGTGGGAAAGGCTTTTGCGATGAGATTAATGCATCTTGACTTCAATGTGT
ATGTCGTGGGCGAGACAATAACCCCGGCCTTCGAAGAGGGCGACCTTCTCATTGCTATCTCCGG
TAGTGGAGAAACAAAGACAATCGTCGACGCCGCGGAGATAGCAAAACAACAGGGCGGTAAAGTC
GTTGCCATAACGAGTTACAAAGACTCGACTTTGGGCAGACTGGCCGATGTAGTTGTAGAAATTC
CAGGGAGAACTAAAACGGACGTCCCGACAGATTATATTGCGAGGCAAATGTTAACTAAGTACAA
ATGGACAGCGCCCATGGGGACCCTATTTGAAGATTCAACTATGATCTTTCTTGACGGGATTATA
GCGCTATTAATGGCGACTTTTCAGAAAACTGAGAAAGACATGAGGAAGAAGCACGCAACTCTAG
AG
-Exemplary Pyrococcus horikoshii OT3 3-hexulose-6-phosphate
formaldehyde lyase (HPS/PHI-archea) Amino Acid Sequence
SEQ ID NO: 171
MILQVALDLTDIEQAISIAEKAARGGAHWLEVGTPLIKKEGMRAVELLKRRFPDRKIVADLKTM
DTGALEVEMAARHGADVVSILGVADDKTIKDALAVARKYGVKIMVDLIGVKDKVQRAKELEQMG
VHYILVHTGIDEQAQGKTPLEDLEKVVKAVKIPVAVAGGLNLETIPKVIELGATIVIVGSAITK
SKDPEGVTRKIIDLFWDEYMKTIRKAMKDITDHINEVADKLRLDEVRGLVDAMIGANKIFIYGA
GRSGLVGKAFAMRLMHLDFNVYVVGETITPAFEEGDLLIAISGSGETKTIVDAAEIAKQQGGKV
VAITSYKDSTLGRLADVVVEIPGRTKTDVPTDYIARQMLTKYKWTAPMGTLFEDSTMIFLDGII
ALLMATFQKTEKDMRKKHATLE
-Exemplary Synthetic 3-hexulose-6-phosphate formaldehyde lyase (HPS-
synthetic) Nucleic Acid Coding Sequence
SEQ ID NO: 172
ATGAAGCTCCAAGTCGCCATCGACCTGCTGTCCACCGAAGCCGCCCTCGAGCTGGCCGGCAAGG
TTGCCGAGTACGTCGACATCATCGAACTGGGCACCCCCCTGATCAAGGCCGAGGGCCTGTCGGT
CATCACCGCCGTCAAGAAGGCTCACCCGGACAAGATCGTCTTCGCCGACATGAAGACCATGGAC
GCCGGCGAGCTCGAAGCCGACATCGCGTTCAAGGCCGGCGCTGACCTGGTCACGGTCCTCGGCT
CGGCCGACGACTCCACCATCGCGGGTGCCGTCAAGGCCGCCCAGGCTCACAACAAGGGCGTCGT
CGTCGACCTGATCGGCATCGAGGACAAGGCCACCCGTGCACAGGAAGTTCGCGCCCTGGGTGCC
AAGTTCGTCGAGATGCACGCTGGTCTGGACGAGCAGGCCAAGCCCGGCTTCGACCTGAACGGTC
TGCTCGCCGCCGGCGAGAAGGCTCGCGTTCCGTTCTCCGTGGCCGGTGGCGTGAAGGTTGCGAC
CATCCCCGCAGTCCAGAAGGCCGGCGCAGAGGTTGCCGTCGCCGGTGGCGCCATCTACGGTGCA
GCCGACCCGGCCGCCGCCGCGAAGGAACTGCGCGCCGCGATCGCCATGACGCAAGCCGCAGAAG
CCGACGGGGCCGTGAAGGTCGTCGGAGACGACATCACCAACAACCTTTCCCTTGTTCGGGACGA
GGTCGCGGACACCGCGGCGAAAGTCGACCCGGAGCAGGTGGCTGTCCTCGCTCGCCAAATCGTC
CAGCCTGGACGGGTTTTCGTGGCGGGCGCCGGTCGCAGCGGGCTCGTCCTGCGCATGGCCGCCA
TGCGGCTGATGCACTTCGGCCTCACCGTGCACGTCGCGGGCGACACCACCACCCCGGCAATCTC
AGCCGGCGATCTGCTGCTGGTGGCTTCCGGCTCGGGCACCACCTCCGGTGTGGTCAAGTCCGCC
GAGACGGCCAAGAAGGCCGGGGCGCGCATCGCCGCCTTCACCACCAACCCGGATTCTCCGCTGG
CCGGTCTGGCCGACGCCGTGGTGATCATCCCCGCCGCGCAGAAGACCGATCACGGCTCGCACAT
TTCGCGGCAGTACGCCGGATCCCTTTTCGAGCAGGTGCTGTTCGTCGTCACCGAAGCCGTGTTC
CAGTCGCTGTGGGATCACACCGAGGTCGAGGCCGAGGAACTCTGGACGCGCCACGCCAACCTCG
AGTGA
-Exemplary Synthetic 3-hexulose-6-phosphate formaldehyde lyase (HPS-
synthetic) Amino Acid Sequence
SEQ ID NO: 173
MKLQVAIDLLSTEAALELAGKVAEYVDIIELGTPLIKAEGLSVITAVKKAHPDKIVFADMKTMD
AGELEADIAFKAGADLVTVLGSADDSTIAGAVKAAQAHNKGVVVDLIGIEDKATRAQEVRALGA
KFVEMHAGLDEQAKPGFDLNGLLAAGEKARVPFSVAGGVKVATIPAVQKAGAEVAVAGGAIYGA
ADPAAAAKELRAAIAMTQAAEADGAVKVVGDDITNNLSLVRDEVADTAAKVDPEQVAVLARQIV
QPGRVFVAGAGRSGLVLRMAAMRLMHFGLTVHVAGDTTTPAISAGDLLLVASGSGTTSGVVKSA
ETAKKAGARIAAFTTNPDSPLAGLADAVVIIPAAQKTDHGSHISRQYAGSLFEQVLFVVTEAVE
QSLWDHTEVEAEELWTRHANLE

### 3-Hexulose-6-Phosphate Synthase (HPS)

In some embodiments, a composition described herein comprises a transgenic HPS protein. In some embodiments, such a protein, among other things, may utilize formaldehyde as a substrate and produce D-arabino-3-hexulose 6-phosphate, (Hu6P). In some embodiments, such a protein, may be fused with a PHI enzyme.

In some embodiments, a HPS gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 175 or 177 (or a portion thereof). In some embodiments, a HPS gene and/or transgene comprises a nucleotide sequence that is at least 90%, 91%, 92%, 93%, 94%, 95%, 96%, 97%, 98%, 99%, or 100% identical to any one of SEQ ID NOs: 174 or 176 (or a portion thereof).

-Exemplary Mycobacterium gastri 3-hexulose-6-phosphate synthase
(HPS-Mg) Nucleic Acid Coding Sequence
SEQ ID NO: 174
ATGAAACTACAAGTTGCGATAGATCTCTTGTCTACAGAAGCAGCTTTGGAATTGGCCGGTAAAG
TGGCTGAGTACGTGGACATCATAGAATTGGGTACGCCCCTGATAGAAGCAGAGGGTCTTTCGGT
AATTACAGCCGTTAAAAAGGCACATCCCGACAAGATTGTTTTCGCCGATATGAAAACCATGGAT
GCAGGTGAACTCGAGGCAGACATTGCATTTAAAGCTGGTGCAGACCTCGTGACTGTTCTTGGGA
GCGCCGACGATTCTACAATTGCAGGCGCAGTTAAAGCAGCCCAAGCCCACAACAAAGGCGTCGT
GGTTGATCTGATCGGCATCGAGGACAAAGCGACCAGAGCCCAAGAAGTGAGAGCATTGGGCGCC
AAGTTTGTTGAGATGCACGCAGGCCTCGATGAACAAGCCAAGCCCGGCTTCGACTTGAACGGTT
TGTTAGCAGCCGGCGAGAAAGCACGCGTTCCTTTTAGTGTAGCAGGTGGCGTTAAGGTCGCTAC
GATCCCTGCTGTCCAAAAAGCTGGTGCGGAAGTGGCAGTTGCGGGCGGTGCCATCTATGGGGCA
GCTGATCCCGCGGCCGCTGCCAAAGAGCTTAGAGCAGCTATAGCC
-Exemplary Mycobacterium gastri 3-hexulose-6-phosphate synthase
(HPS-Mg) Amino Acid Sequence
SEQ ID NO: 175
MKLQVAIDLLSTEAALELAGKVAEYVDIIELGTPLIEAEGLSVITAVKKAHPDKIVFADMKTMD
AGELEADIAFKAGADLVTVLGSADDSTIAGAVKAAQAHNKGVVVDLIGIEDKATRAQEVRALGA
KFVEMHAGLDEQAKPGFDLNGLLAAGEKARVPFSVAGGVKVATIPAVQKAGAEVAVAGGAIYGA
ADPAAAAKELRAAIA
-Exemplary Bacillus methanolicus MGA3 3-hexulose-6-phosphate
synthase (HPS-Bm) Nucleic Acid Coding Sequence
SEQ ID NO: 176
ATGGAACTACAGTTGGCATTAGACTTAGTCAACATTGAAGAGGCAAAGCAAGTGGTTGCGGAAG
TCCAAGAGTATGTGGATATTGTGGAGATTGGAACTCCAGTAATAAAGATATGGGGTTTGCAAGC
AGTCAAAGCTGTTAAGGATGCGTTCCCACATCTGCAAGTTTTGGCCGATATGAAAACGATGGAT
GCAGCCGCATACGAAGTAGCTAAAGCGGCCGAGCACGGAGCTGACATCGTTACGATTCTTGCAG
CGGCCGAGGACGTGTCTATCAAAGGTGCAGTTGAAGAGGCGAAAAAGTTAGGAAAGAAAATACT
GGTGGACATGATTGCCGTTAAAAATTTAGAGGAAAGAGCCAAGCAGGTAGATGAGATGGGGGTC
GACTATATATGTGTACATGCAGGGTATGACTTGCAGGCTGTTGGAAAAAATCCCTTAGATGACC
TAAAGAGGATAAAAGCCGTGGTTAAGAACGCTAAAACTGCGATCGCAGGGGGAATCAAACTCGA
AACGTTACCCGAGGTTATCAAAGCAGAACCAGATCTAGTGATTGTGGGAGGGGGCATTGCAAAC
CAAACAGACAAGAAAGCTGCAGCTGAAAAGATTAATAAACTTGTGAAACAGGGCCTT
-Exemplary Bacillus methanolicus MGA3 3-hexulose-6-phosphate
synthase (HPS-Bm) Amino Acid Sequence
SEQ ID NO: 177
MELQLALDLVNIEEAKQVVAEVQEYVDIVEIGTPVIKIWGLQAVKAVKDAFPHLQVLADMKTMD
AAAYEVAKAAEHGADIVTILAAAEDVSIKGAVEEAKKLGKKILVDMIAVKNLEERAKQVDEMGV
DYICVHAGYDLQAVGKNPLDDLKRIKAVVKNAKTAIAGGIKLETLPEVIKAEPDLVIVGGGIAN
QTDKKAAAEKINKLVKQGL

### 6-Phospho-3-Hexuloisomerase (PHI)

In some embodiments, a composition described herein comprises a transgenic PHI protein. In some embodiments, such a protein, among other things, may utilize D-arabino-3-hexulose 6-phosphate (Hu6P) as a substrate and produce fructose 6-phosphate (F6P). In some embodiments, such a protein, may be fused with a HPS enzyme.

In some embodiments, a PHI gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 179 or 181 (or a portion thereof). In some embodiments, a PHI gene and/or transgene comprises a nucleotide sequence that is at least 90%, 91%, 92%, 93%, 94%, 95%, 96%, 97%, 98%, 99%, or 100% identical to any one of SEQ ID NOs: 178 or 180 (or a portion thereof).

-Exemplary Bacillus methanolicus MGA3 6-phospho-3-hexuloisomerase
(PHI-Bm) Nucleic Acid Coding Sequence
SEQ ID NO: 178
ATGATTTCCATGCTTACCACTGAATTTCTGGCAGAAATAGTGAAAGAGTTGAACAGTAGCGTAA
ATCAAATCGCAGACGAAGAGGCTGAAGCGCTGGTTAACGGCATATTGCAATCGAAGAAAGTGTT
CGTGGCGGGAGCTGGTCGTTCCGGGTTCATGGCGAAGTCATTCGCCATGAGGATGATGCACATG
GGGATCGATGCTTATGTGGTCGGAGAGACAGTGACACCAAATTATGAGAAAGAGGATATCCTTA
TAATTGGGTCAGGGTCAGGGGAAACCAAAAGTTTGGTTTCAATGGCTCAGAAAGCGAAAAGCAT
CGGGGGCACAATTGCAGCGGTGACAATTAATCCTGAGTCTACCATCGGTCAATTGGCTGATATA
GTAATAAAAATGCCCGGATCTCCAAAAGACAAATCTGAAGCCAGGGAAACAATCCAACCAATGG
GATCTCTTTTCGAGCAAACTCTTTTGCTCTTTTACGACGCCGTAATACTTAGATTTATGGAAAA
GAAAGGACTTGACACCAAAACAATGTACGGTAGGCACGCAAATTTGGAGTGA
-Exemplary Bacillus methanolicus MGA3 6-phospho-3-hexuloisomerase
(PHI-Bm) Amino Acid Sequence
SEQ ID NO: 179
MISMLTTEFLAEIVKELNSSVNQIADEEAEALVNGILQSKKVFVAGAGRSGFMAKSFAMRMMHM
GIDAYVVGETVTPNYEKEDILIIGSGSGETKSLVSMAQKAKSIGGTIAAVTINPESTIGQLADI
VIKMPGSPKDKSEARETIQPMGSLFEQTLLLFYDAVILRFMEKKGLDTKTMYGRHANLE
-Exemplary Mycobacterium gastri 6-phospho-3-hexuloisomerase (PHI-Mg)
Nucleic Acid Coding Sequence
SEQ ID NO: 180
ATGACCCAAGCGGCAGAAGCAGACGGCGCGGTCAAAGTAGTTGGCGATGACATAACTAACAATC
TGAGCCTAGTAAGGGATGAAGTCGCCGATACAGCAGCCAAGGTGGACCCAGAACAAGTGGCTGT
CCTCGCAAGGCAGATCGTGCAGCCTGGTAGGGTGTTTGTGGCTGGCGCAGGACGAAGCGGACTG
GTTCTGCGGATGGCTGCCATGAGACTTATGCATTTTGGACTGACCGTGCATGTGGCCGGGGATA
CGACTACGCCTGCCATTTCTGCAGGGGACTTGCTTTTAGTCGCTAGTGGGTCAGGGACCACATC
TGGAGTGGTTAAAAGTGCTGAGACAGCTAAGAAAGCAGGGGCAAGAATCGCAGCCTTTACAACT
AATCCAGATAGTCCGCTCGCCGGACTTGCAGATGCCGTGGTTATCATACCTGCTGCGCAGAAAA
CGGATCATGGGTCGCATATATCACGGCAATATGCTGGCAGTCTCTTTGAGCAGGTTCTCTTTGT
GGTTACCGAGGCCGTCTTTCAATCACTCTGGGACCACACTGAAGTCGAAGCTGAGGAACTATGG
ACACGGCACGCTAATCTAGAATAG
-Exemplary Mycobacterium gastri 6-phospho-3-hexuloisomerase (PHI-Mg)
Amino Acid Sequence
SEQ ID NO: 181
MTQAAEADGAVKVVGDDIINNLSLVRDEVADTAAKVDPEQVAVLARQIVQPGRVFVAGAGRSGL
VLRMAAMRLMHFGLTVHVAGDTTTPAISAGDLLLVASGSGTTSGVVKSAETAKKAGARIAAFTT
NPDSPLAGLADAVVIIPAAQKTDHGSHISRQYAGSLFEQVLFVVTEAVFQSLWDHTEVEAEELW
TRHANLE

### Synthetic Acetyl-CoA Enzymes (SACA)

In certain embodiments, a composition described herein comprises at least one transgenic SACA pathway enzyme. In some embodiments, such enzymes metabolize substrates such as formaldehyde, glycoaldehyde, and/or acetylphosphate to create products such as glycoaldehyde, acetylphosphate, and/or acetylCOA. In certain embodiments, acetylCOA is further utilized in the citric acid cycle.

In some embodiments, a SACA gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 182, 184, or 186 (or a portion thereof). In some embodiments, a SACA gene and/or transgene comprises a nucleotide sequence that is at least 90%, 91%, 92%, 93%, 94%, 95%, 96%, 97%, 98%, 99%, or 100% identical to any one of SEQ ID NOs: 183 or 185 (or a portion thereof).

-Exemplary Pseudomonas putida glycolaldehyde synthase (GALS) Amino
Acid Sequence
SEQ ID NO: 182
MGSSHHHHHHSSGLVPRGSHMMASVHGTTYELLRRQGIDTVFGNPGSNELPFLKDFPEDFRYIL
ALQEACVVGIADGYAQASRKPAFINLHSAAGTGNAMGALSNARTSHSPLIVTAGQQTRAMIGVE
AGETNVDAANLPRPLVKWSYEPASAAEVPHAMSRAIHMASMAPQGPVYLSVPYDDWDKDADPQS
HHLFDRHVSSSVRLNDQDLDILVKALNSASNPAIVLGPDVDAANANADCVMLAERLKAPVWVAP
SAPRCPFPTRHPCFRGLMPAGIAAISQLLEGHDVVLVIGAPVFRYVFYDPGQYLKPGTRLISVT
CDPLEAARAPMGDAIVADIGAMASALANLVEESSRQLPTAAPEPAKVDQDAGRLHPETVEDTLN
DMAPENAIYLNESTSTTAQMWQRLNMRNPGSYYFCAAGGLGFALPAAIGVQLAEPERQVIAVIG
DGSANYSISALWTAAQYNIPTIFVIMNNGTYGMLRWFAGVLEAENVPGLDVPGIDFRALAKGYG
VQALKADNLEQLKGSLQEALSAKGPVLIEVSTVSPVK
-Exemplary Bifidobacterium breve acetyl-phosphate synthase
(phosphoketolase) (ACPS) Nucleic Acid Coding Sequence
SEQ ID NO: 183
ATGACAAATCCTGTTATTGGCACCCCGTGGCAGAAGCTGGATCGCCCGGTTTCCGAAGAAGCCA
TCGAAGGCATGGACAAGTATTGGCGCGTCACCAACTACATGTCCATCGGCCAGATCTATCTGCG
TAGCAACCCGCTGATGAAGGAACCCTTCACCCGCGATGACGTGAAGCACCGTCTGGTCGGCCAC
TGGGGCACCACCCCGGGCCTGAACTTCCTTCTCGCCCACATCAACCGCCTCATCGCTGACCACC
AGCAGAACACCGTGTTCATCATGGGCCCGGGCCACGGCGGCCCGGCTGGCACCTCCCAGTCTTA
CGTTGACGGCACGTACACCGAGTACTACCCGAACATCACCAAGGACGAAGCTGGCCTGCAGAAG
TTCTTCCGCCAGTTCTCCTACCCGGGCGGCATCCCGTCGCACTTCGCCCCGGAGACCCCGGGAT
CGATCCACGAAGGTGGCGAGCTTGGCTACGCGCTCTCCCACGCATACGGCGCCGTGATGAACAA
CCCGAGCCTGTTCGTGCCGTGCATCATCGGCGACGGCGAGGCCGAGACCGGCCCGCTCGCCACC
GGCTGGCAGTCCAACAAGCTCGTCAACCCGCGCACCGACGGCATCGTGCTGCCGATCCTGCACC
TCAACGGCTACAAGATCGCCAACCCGACCATCCTCGCTCGTATCTCCGACGAAGAGCTGCATGA
CTTCTTCCGCGGCATGGGCTACCACCCGTACGAGTTCGTTGCCGGCTTCGACAACGAGGACCAC
ATGTCGATCCACCGTCGTTTCGCCGAGCTGTTCGAGACGATCTTCGACGAGATCTGCGACATCA
AGGCTGCGGCCCAGACCGACGACATGACCCGTCCGTTCTACCCGATGCTCATCTTCCGCACCCC
GAAGGGCTGGACCTGCCCGAAGTTCATCGACGGCAAGAAGACCGAAGGCTCCTGGCGTGCGCAC
CAGGTCCCGCTGGCTTCCGCCCGCGACACCGAAGAGCACTTCGAAGTCCTCAAGGGCTGGATGG
AATCCTACAAGCCGGAAGAGCTCTTCAACGCCGACGGCTCCATCAAGGATGACGTCACCGCGTT
CATGCCGAAGGGCGAGCTCCGCATCGGCGCCAACCCGAACGCCAACGGTGGTGTGATCCGCGAG
GACCTGAAGCTCCCCGAGCTCGACCAGTACGAGGTCACCGGCGTCAAGGAGTACGGCCATGGCT
GGGGCCAGGTCGAGGCTCCGCGTGCCCTCGGTGCATACTGCCGCGACATCATCAAGAACAACCC
GGATTCGTTCCGCATCTTCGGACCGGACGAGACCGCTTCCAACCGCCTGAACGCGACCTACGAG
GTCACCGACAAGCAGTGGGACAACGGCTACCTTTCGGGTCTCGTCGACGAGCACATGGCGGTCA
CCGGTCAGGTCACCGAGCAGCTCTCCGAGCACCAGTGCGAGGGCTTCCTCGAGGCGTACCTCCT
CACCGGCCGCCACGGCATCTGGAGCTCCTACGAGTCCTTCGTCCACGTCATCGACTCGATGCTC
AACCAGCATGCGAAGTGGCTCGAGGCCACCGTCCGCGAGATCCCGTGGCGCAAGCCGATCTCCT
CGGTGAACCTCCTCGTCTCCTCGCACGTGTGGCGTCAGGATCACAACGGCTTCTCGCACCAGGA
TCCGGGTGTCACCTCGCTCCTGATCAACAAGACGTTCAACAACGATCACGTGACGAACATCTAC
TTCGCGACCGACGCGAACATGCTGCTCGCGATCTCCGAGAAGTGCTTCAAGTCCACCAACAAGA
TCAATGCGATCTTCGCCGGCAAGCAGCCTGCTCCGACGTGGGTCACGCTCGATGAGGCCCGCGC
CGAGCTCGAAGCCGGCGCCGCTGAGTGGAAGTGGGCTTCCAACGCCGAGAACAACGATGAGGTC
CAGGTCGTCCTCGCTTCCGCTGGCGATGTGCCGACCCAGGAGCTCATGGCCGCCTCCGATGCCC
TCAACAAGATGGGCATCAAGTTCAAGGTCGTCAACGTTGTTGACCTCCTGAAGCTGCAGTCCCG
CGAGAACAACGACGAGGCCCTCACGGACGAGGAGTTCACCGAACTCTTCACCGCCGACAAGCCG
GTTCTGTTCGCATACCACTCCTACGCTCAGGATGTTCGCGGCCTCATCTACGACCGCCCGAACC
ACGACAACTTCCACGTCGTCGGCTACAAGGAGCAGGGCTCCACGACCACGCCGTTCGACATGGT
CCGCGTCAACGACATGGATCGCTATGCGCTCCAGGCCGCTGCCCTCAAGCTGATCGATGCCGAC
AAGTACGCCGACAAGATCGACGAGCTCAACGCGTTCCGCAAGAAGGCGTTCCAGTTCGCTGTCG
ACAACGGCTACGACATCCCGGAGTTCACCGACTGGGTGTACCCGGATGTCAAGGTCGACGAGAC
GCAGATGCTTTCCGCGACCGCGGCGACCGCAGGCGACAACGAGTGA
-Exemplary Bifidobacterium breve acetyl-phosphate synthase
(phosphoketolase) (ACPS) Amino Acid Sequence
SEQ ID NO: 184
MTNPVIGTPWQKLDRPVSEEAIEGMDKYWRVTNYMSIGQIYLRSNPLMKEPFTRDDVKHRLVGH
WGTTPGLNFLLAHINRLIADHQQNTVFIMGPGHGGPAGTSQSYVDGTYTEYYPNITKDEAGLQK
FFRQFSYPGGIPSHFAPETPGSIHEGGELGYALSHAYGAVMNNPSLFVPCIIGDGEAETGPLAT
GWQSNKLVNPRTDGIVLPILHLNGYKIANPTILARISDEELHDFFRGMGYHPYEFVAGFDNEDH
MSIHRRFAELFETIFDEICDIKAAAQTDDMTRPFYPMLIFRTPKGWTCPKFIDGKKTEGSWRAH
QVPLASARDTEEHFEVLKGWMESYKPEELFNADGSIKDDVTAFMPKGELRIGANPNANGGVIRE
DLKLPELDQYEVTGVKEYGHGWGQVEAPRALGAYCRDIIKNNPDSFRIFGPDETASNRLNATYE
VTDKQWDNGYLSGLVDEHMAVTGQVTEQLSEHQCEGFLEAYLLTGRHGIWSSYESFVHVIDSML
NQHAKWLEATVREIPWRKPISSVNLLVSSHVWRQDHNGFSHQDPGVTSLLINKTENNDHVTNIY
FATDANMLLAISEKCFKSTNKINAIFAGKQPAPTWVTLDEARAELEAGAAEWKWASNAENNDEV
QVVLASAGDVPTQELMAASDALNKMGIKFKVVNVVDLLKLQSRENNDEALTDEEFTELFTADKP
VLFAYHSYAQDVRGLIYDRPNHDNFHVVGYKEQGSTTTPFDMVRVNDMDRYALQAAALKLIDAD
KYADKIDELNAFRKKAFQFAVDNGYDIPEFTDWVYPDVKVDETQMLSATAATAGDNE
-Exemplary Escherichia coli phosphate acetyltransferase (PTA) Nucleic
Acid Coding Sequence
SEQ ID NO: 185
ATGTCCCGTATTATTATGCTGATCCCTACCGGAACCAGCGTCGGTCTGACCAGCGTCAGCCTTG
GCGTGATCCGTGCAATGGAACGCAAAGGCGTTCGTCTGAGCGTTTTCAAACCTATCGCTCAGCC
GCGTACCGGTGGCGATGCGCCCGATCAGACTACGACTATCGTGCGTGCGAACTCTTCCACCACG
ACGGCCGCTGAACCGCTGAAAATGAGCTACGTTGAAGGTCTGCTTTCCAGCAATCAGAAAGATG
TGCTGATGGAAGAGATCGTCGCAAACTACCACGCTAACACCAAAGACGCTGAAGTCGTTCTGGT
TGAAGGTCTGGTCCCGACACGTAAGCACCAGTTTGCCCAGTCTCTGAACTACGAAATCGCTAAA
ACGCTGAATGCGGAAATCGTCTTCGTTATGTCTCAGGGCACTGACACCCCGGAACAGCTGAAAG
AGCGTATCGAACTGACCCGCAACAGCTTCGGCGGTGCCAAAAACACCAACATCACCGGCGTTAT
CGTTAACAAACTGAACGCACCGGTTGATGAACAGGGTCGTACTCGCCCGGATCTGTCCGAGATT
TTCGACGACTCTTCCAAAGCTAAAGTAAACAATGTTGATCCGGCGAACGTGCAAGAATCCAGCC
CGCTGCCGGTTCTCGGCGCTGTGCCGTGGAGCTTTGACCTGATCGCGACTCGTGCGATCGATAT
GGCTCGCCACCTGAATGCGACCATCATCAACGAAGGCGACATCAATACTCGCCGCGTTAAATCC
GTCACTTTCTGCGCACGCAGCATTCCGCACATGCTGGAGCACTTCCGTGCCGGTTCTCTGCTGG
TGACTTCCGCAGACCGTCCTGACGTGCTGGTGGCCGCTTGCCTGGCAGCCATGAACGGCGTAGA
AATCGGTGCCCTGCTGCTGACTGGCGGTTACGAAATGGACGCGCGCATTTCTAAACTGTGCGAA
CGTGCTTTCGCTACCGGCCTGCCGGTATTTATGGTGAACACCAACACCTGGCAGACCTCTCTGA
GCCTGCAGAGCTTCAACCTGGAAGTTCCGGTTGACGATCACGAACGTATCGAGAAAGTTCAGGA
ATACGTTGCTAACTACATCAACGCTGACTGGATCGAATCTCTGACTGCCACTTCTGAGCGCAGC
CGTCGTCTGTCTCCGCCTGCGTTCCGTTATCAGCTGACTGAACTTGCGCGCAAAGCGGGCAAAC
GTATCGTACTGCCGGAAGGTGACGAACCGCGTACCGTTAAAGCAGCCGCTATCTGTGCTGAACG
TGGTATCGCAACTTGCGTACTGCTGGGTAATCCGGCAGAGATCAACCGTGTTGCAGCGTCTCAG
GGTGTAGAACTGGGTGCAGGGATTGAAATCGTTGATCCAGAAGTGGTTCGCGAAAGCTATGTTG
GTCGTCTGGTCGAACTGCGTAAGAACAAAGGCATGACCGAAACCGTTGCCCGCGAACAGCTGGA
AGACAACGTGGTGCTCGGTACGCTGATGCTGGAACAGGATGAAGTTGATGGTCTGGTTTCCGGT
GCTGTTCACACTACCGCAAACACCATCCGTCCGCCGCTGCAGCTGATCAAAACTGCACCGGGCA
GCTCCCTGGTATCTTCCGTGTTCTTCATGCTGCTGCCGGAACAGGTTTACGTTTACGGTGACTG
TGCGATCAACCCGGATCCGACCGCTGAACAGCTGGCAGAAATCGCGATTCAGTCCGCTGATTCC
GCTGCGGCCTTCGGTATCGAACCGCGCGTTGCTATGCTCTCCTACTCCACCGGTACTTCTGGTG
CAGGTAGCGACGTAGAAAAAGTTCGCGAAGCAACTCGTCTGGCGCAGGAAAAACGTCCTGACCT
GATGATCGACGGTCCGCTGCAGTACGACGCTGCGGTAATGGCTGACGTTGCGAAATCCAAAGCG
CCGAACTCTCCGGTTGCAGGTCGCGCTACCGTGTTCATCTTCCCGGATCTGAACACCGGTAACA
CCACCTACAAAGCGGTACAGCGTTCTGCCGACCTGATCTCCATCGGGCCGATGCTGCAGGGTAT
GCGCAAGCCGGTTAACGACCTGTCCCGTGGCGCACTGGTTGACGATATCGTCTACACCATCGCG
CTGACTGCGATTCAGTCTGCACAGCAGCAGTAA
-Exemplary Escherichia coli phosphate acetyltransferase (PTA) Amino
Acid Sequence
SEQ ID NO: 186
MSRIIMLIPTGTSVGLTSVSLGVIRAMERKGVRLSVFKPIAQPRTGGDAPDQTTTIVRANSSTT
TAAEPLKMSYVEGLLSSNQKDVLMEEIVANYHANTKDAEVVLVEGLVPTRKHQFAQSLNYEIAK
TLNAEIVFVMSQGTDTPEQLKERIELTRNSFGGAKNTNITGVIVNKLNAPVDEQGRTRPDLSEI
FDDSSKAKVNNVDPAKLQESSPLPVLGAVPWSEDLIATRAIDMARHLNATIINEGDINTRRVKS
VTFCARSIPHMLEHFRAGSLLVTSADRPDVLVAACLAAMNGVEIGALLLIGGYEMDARISKLCE
RAFATGLPVFMVNTNTWQTSLSLQSFNLEVPVDDHERIEKVQEYVANYINADWIESLTATSERS
RRLSPPAFRYQLTELARKAGKRIVLPEGDEPRTVKAAAICAERGIATCVLLGNPAEINRVAASQ
GVELGAGIEIVDPEVVRESYVGRLVELRKNKGMTETVAREQLEDNVVLGTLMLEQDEVDGLVSG
AVHTTANTIRPPLQLIKTAPGSSLVSSVFFMLLPEQVYVYGDCAINPDPTAEQLAEIAIQSADS
AAAFGIEPRVAMLSYSTGTSGAGSDVEKVREATRLAQEKRPDLMIDGPLQYDAAVMADVAKSKA
PNSPVAGRATVFIFPDLNTGNTTYKAVQRSADLISIGPMLQGMRKPVNDLSRGALVDDIVYTIA
LTAIQSAQQQ

### B) Propanediol Pathway Enzymes (Aldolase)

In certain embodiments, a composition described herein comprises at least one transgenic aldolase pathway enzyme. In certain embodiments, aldolase enzymes metabolize substrates such as formaldehyde, pyruvate, 2-keto-4-hydroxybutyrate (HOBA), and/or 3-hydroxypropionaldehyde (3-HPA) to create products such as 2-keto-4-hydroxybutyrate (HOBA), 3-hydroxypropionaldehyde (3-HPA), and/or 1,3-propanediol (1,3-PDO). In certain embodiments, 1,3-PDO is further utilized in metabolic processes in the host cell.

In some embodiments, an aldolase gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 188, 190, or 192 (or a portion thereof). In some embodiments, an aldolase gene and/or transgene comprises a nucleotide sequence that is at least 90%, 91%, 92%, 93%, 94%, 95%, 96%, 97%, 98%, 99%, or 100% identical to any one of SEQ ID NOs: 187, 189, or 191 (or a portion thereof).

-Exemplary Escherichia coli K-12, 4-hydroxy-2-oxoglutarate aldolase/2-
dehydro-3-deoxy-phosphogluconate aldolase (KHB) Nucleic Acid Coding Sequence
SEQ ID NO: 187
ATGAAAAACTGGAAAACAAGTGCAGAATCAATCCTGACCACCGGCCCGGTTGTACCGGTTATCG
TGGTAAAAAAACTGGAACACGCGGTGCCGATGGCAAAAGCGTTGGTTGCTGGTGGGGTGCGCGT
TCTGGAAGTGACTCTGCGTACCGAGTGTGCAGTTGACGCTATCCGTGCTATCGCCAAAGAAGTG
CCTGAAGCGATTGTGGGTGCCGGTACGGTGCTGAATCCACAGCAGCTGACAGAAGTCACTGAAG
CGGGTGCACAGTTCGCAATTAGCCCGGGTCTGACCGAGCCGCTGCTGAAAGCTGCTACCGAAGG
GACTATTCCTCTGATTCCGGGGATCAGCACTGTTTCCGAACTGATGCTGGGTATGGACTACGGT
TTGAAAGAGTTCAAATTCTTCCCGGCTGAAGCTAACGGCGGCGTGAAAGCCCTGCAGGCGATCG
CGGGTCCGTTCTCCCAGGTCCGTTTCTGCCCGACGGGTGGTATTTCTCCGGCTAACTACCGTGA
CTACCTGGCGCTGAAAAGCGTGCTGTGCATCGGTGGTTCCTGGCTGGTTCCGGCAGATGCGCTG
GAAGCGGGCGATTACGACCGCATTACTAAGCTGGCGCGTGAAGCTGTAGAAGGCGCTAAGCTGT
AA
-Exemplary Escherichia coli K-12, 4-hydroxy-2-oxoglutarate aldolase/2-
dehydro-3-deoxy-phosphogluconate aldolase (KHB) Amino Acid Sequence
SEQ ID NO: 188
MKNWKTSAESILTTGPVVPVIVVKKLEHAVPMAKALVAGGVRVLEVTLRTECAVDAIRAIAKEV
PZAIVGAGTVLNPQQLAEVTEAGAQFAISPGLTEPLLKAATEGTIPLIPGISTVSELMLGMDYG
LKEFKFFPAEANGGVKALQAIAGPFSQVRFCPTGGISPANYRDYLALKSVLCIGGSWLVPADAL
EAGDYDRITKLAREAVEGAKL
-Exemplary Lactococcus lactis branched-chain alpha-keto acid
decarboxylase (KDC) Nucleic Acid Coding Sequence
SEQ ID NO: 189
ATGTATACAGTAGGAGATTACCTGTTAGACCGATTACACGAGTTGGGAATTGAAGAAATTTTTG
GAGTTCCTGGTGACTATAACTTACAATTTTTAGATCAAATTATTTCACGCGAAGATATGAAATG
GATTGGAAATGCTAATGAATTAAATGCTTCTTATATGGCTGATGGTTATGCTCGTACTAAAAAA
GCTGCCGCATTTCTCACCACATTTGGAGTCGGCGAATTGAGTGCGATCAATGGACTGGCAGGAA
GTTATGCCGAAAATTTACCAGTAGTAGAAATTGTTGGTTCACCAACTTCAAAAGTACAAAATGA
CGGAAAATTTGTCCATCATACACTAGCAGATGGTGATTTTAAACACTTTATGAAGATGCATGAA
CCTGTTACAGCAGCGCGGACTTTACTGACAGCAGAAAATGCCACATATGAAATTGACCGAGTAC
TTTCTCAATTACTAAAAGAAAGAAAACCAGTCTATATTAACTTACCAGTCGATGTTGCTGCAGC
AAAAGCAGAGAAGCCTGCATTATCTTTAGAAAAAGAAAGCTCTACAACAAATACAACTGAACAA
GTGATTTTGAGTAAGATTGAAGAAAGTTTGAAAAATGCCCAAAAACCAGTAGTGATTGCAGGAC
ACGAAGTAATTAGTTTTGGTTTAGAAAAAACGGTAACTCAGTTTGTTTCAGAAACAAAACTACC
GATTACGACACTAAATTTTGGTAAAAGTGCTGTTGATGAATCTTTGCCCTCATTTTTAGGAATA
TATAACGGGAAACTTTCAGAAATCAGTCTTAAAAATTTTGTGGAGTCCGCAGACTTTATCCTAA
TGCTTGGAGTGAAGCTTACGGACTCCTCAACAGGTGCATTCACACATCATTTAGATGAAAATAA
AATGATTTCACTAAACATAGATGAAGGAATAATTTTCAATAAAGTGGTAGAAGATTTTGATTTT
AGAGCAGTGGTTTCTTCTTTATCAGAATTAAAAGGAATAGAATATGAAGGACAATATATTGATA
AGCAATATGAAGAATTTATTCCATCAAGTGCTCCCTTATCACAAGACCGTCTATGGCAGGCAGT
TGAAAGTTTGACTCAAAGCAATGAAACAATCGTTGCTGAACAAGGAACCTCATTTTTTGGAGCT
TCAACAATTTTCTTAAAATCAAATAGTCGTTTTATTGGACAACCTTTATGGGGTTCTATTGGAT
ATACTTTTCCAGCGGCTTTAGGAAGCCAAATTGCGGATAAAGAGAGCAGACACCTTTTATTTAT
TGGTGATGGTTCACTTCAACTTACCGTACAAGAATTAGGACTATCAATCAGAGAAAAACTCAAT
CCAATTTGTTTTATCATAAATAATGATGGTTATACAGTTGAAAGAGAAATCCACGGACCTACTC
AAAGTTATAACGACATTCCAATGTGGAATTACTCGAAATTACCAGAAACATTTGGAGCAACAGA
AGATCGTGTAGTATCAAAAATTGTTAGAACAGAGAATGAATTTGTGTCTGTCATGAAAGAAGCC
CAAGCAGATGTCAATAGAATGTATTGGATAGAACTAGTTTTGGAAAAAGAAGATGCGCCAAAAT
TACTGAAAAAAATGGGTAAATTATTTGCTGAGCAAAATAAATAG
-Exemplary Lactococcus lactis branched-chain alpha-keto acid
decarboxylase (KDC) Amino Acid Sequence
SEQ ID NO: 190
MYTVGDYLLDRLHELGIEEIFGVPGDYNLQFLDQIISREDMKWIGNANELNASYMADGYARTKK
AAAFLTTFGVGELSAINGLAGSYAENLPVVEIVGSPTSKVQNDGKFVHHTLADGDFKHFMKMHE
PVTAARTLLTAENATYEIDRVLSQLLKERKPVYINLPVDVAAAKAEKPALSLEKESSTINTTEQ
VILSKIEESLKNAQKPVVIAGHEVISFGLEKTVTQFVSETKLPITTLNFGKSAVDESLPSFLGI
YNGKLSEISLKNFVESADFILMLGVKLTDSSTGAFTHHLDENKMISLNIDEGIIFNKVVEDFDF
RAVVSSLSELKGIEYEGQYIDKQYEEFIPSSAPLSQDRLWQAVESLTQSNETIVAEQGTSFFGA
STIFLKSNSRFIGQPLWGSIGYTFPAALGSQIADKESRHLLFIGDGSLQLTVQELGLSIREKLN
PICFIINNDGYTVEREIHGPTQSYNDIPMWNYSKLPETFGATEDRVVSKIVRTENEFVSVMKEA
QADVNRMYWIELVLEKEDAPKLLKKMGKLFAEQNK
-Exemplary K. pneumoniae DSM 2026 NADH-dependent 1,3-PDO
oxidoreductase (DhaT) Nucleic Acid Coding Sequence
SEQ ID NO: 191
ATGAGCTATCGTATGTTTGATTATCTGGTGCCAAACGTTAACTTTTTTGGCCCCAACGCCATTT
CCGTAGTCGGCGAACGCTGCCAGCTGCTGGGGGGGAAAAAAGCCCTGCTGGTCACCGACAAAGG
CCTGCGGGCAATTAAAGATGGCGCGGTGGACAAAACCCTGCATTATCTGCGGGAGGCCGGGATC
GAGGTGGCGATCTTTGACGGCGTCGAGCCGAACCCGAAAGACACCAACGTGCGCGACGGCCTCG
CCGTGTTTCGCCGCGAACAGTGCGACATCATCGTCACCGTGGGCGGCGGCAGCCCGCACGATTG
CGGCAAAGGCATCGGCATCGCCGCCACCCATGAGGGCGATCTGTACCAGTATGCCGGAATCGAG
ACCCTGACCAACCCGCTGCCGCCTATCGTCGCGGTCAATACCACCGCCGGCACCGCCAGCGAGG
TCACCCGCCACTGCGTCCTGACCAACACCGAAACCAAAGTGAAGTTTGTGATCGTCAGCTGGCG
CAACCTGCCGTCGGTCTCTATCAACGATCCACTGCTGATGATCGGTAAACCGGCCGCCCTGACC
GCGGCGACCGGGATGGATGCCCTGACCCACGCCGTAGAGGCCTATATCTCCAAAGACGCTAACC
CGGTGACGGACGCCGCCGCCATGCAGGCGATCCGCCTCATCGCCCGCAACCTGCGCCAGGCCGT
GGCCCTCGGCAGCAATCTGCAGGCGCGGGAAAACATGGCCTATGCTTCTCTGCTGGCCGGGATG
GCTTTCAATAACGCCAACCTCGGCTACGTGCACGCCATGGCGCACCAGCTGGGCGGCCTGTACG
ACATGCCGCACGGCGTGGCCAACGCTGTCCTGCTGCCGCATGTGGCGCGCTACAACCTGATCGC
CAACCCGGAGAAATTCGCCGATATCGCTGAACTGATGGGCGAAAATATCACCGGACTGTCCACT
CTCGACGCGGCGGAAAAAGCCATCGCCGCTATCACGCGTCTGTCGATGGATATCGGTATTCCGC
AGCATCTGCGCGATCTGGGGGTAAAAGAGGCCGACTTCCCCTACATGGCGGAGATGGCTCTAAA
AGACGGCAATGCGTTCTCGAACCCGCGTAAAGGCAACGAGCAGGAGATTGCCGCGATTTTCCGC
CAGGCATTCTGA
-Exemplary K. pneumoniae DSM 2026 NADH-dependent 1,3-PDO
oxidoreductase (DhaT) Amino Acid Sequence
SEQ ID NO: 192
MSYRMFDYLVPNVNFFGPNAISVVGERCQLLGGKKALLVTDKGLRAIKDGAVDKTLHYLREAGI
EVAIFDGVEPNPKDTNVRDGLAVFRREQCDIIVTVGGGSPHDCGKGIGIAATHEGDLYQYAGIE
TLTNPLPPIVAVNTTAGTASEVTRHCVLINTETKVKFVIVSWRNLPSVSINDPLLMIGKPAALT
AATGMDALTHAVEAYISKDANPVTDAAAMQAIRLIARNLRQAVALGSNLQAREYMAYASLLAGM
AFNNANLGYVHAMAHQLGGLYDMPHGVANAVLLPHVARYNLIANPEKFADIAELMGENITGLST
LDAAEKAIAAITRLSMDIGIPQHLRDLGVKETDFPYMAEMALKDGNAFSNPRKGNEQEIAAIFR
QAF

### C) Methanol or Aldehyde Dehydrogenase Enzymes

In certain embodiments, a composition described herein comprises at least one transgenic methanol and/or aldehyde dehydrogenase enzyme. In certain embodiments, methanol and/or aldehyde dehydrogenase enzymes metabolize substrates such as formaldehyde, and/or aldehyde to create products such as methanol, and/or carboxylate. In certain embodiments, methanol, and/or carboxylate is further utilized in metabolic processes in the host cell.

In some embodiments, a methanol and/or aldehyde dehydrogenase gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 194, 196, or 198 (or a portion thereof). In some embodiments, a methanol and/or aldehyde dehydrogenase gene and/or transgene comprises a nucleotide sequence that is at least 90%, 91%, 92%, 93%, 94%, 95%, 96%, 97%, 98%, 99%, or 100% identical to any one of SEQ ID NOs: 193, 195, or 197 (or a portion thereof).

-Exemplary Methylobacterium sp. XJLW Methanol dehydrogenase
(MDH-12) Nucleic Acid Coding Sequence
SEQ ID NO: 193
ATGAGAGCGGTACATCTCCTTGCGCTCGGCGCAGGTGTCGCGGCCGTCGCCGCGCCGGCGCTGG
CCAATGAAAGCGTCATGAAGGGCATCGCCAACCCGGCGGAACAGGTTCTTCAGACGGTTGATTA
CGCGAATACGCGTTATTCGAAGCTCGACCAGATCAACGCCAAGAACGTCAAGGATCTCCAGGTC
GCCTGGACGTTCTCGACCGGCGTTCTGCGCGGCCACGAGGGCTCGCCGCTCGTCGTCGGCAACA
TCATGTACGTGCACACGCCGTTCCCGAACATCGTGTACGCCCTCGACCTCGACCACGAGGCGAA
GATCATCTGGAAGTACGAGCCGAAGCAGGATCCGTCCGTGATCCCGGTCATGTGCTGTGACACG
GTCAACCGTGGCCTGGCCTACGCCGACGGCGCCATCCTCCTGCACCAGGCCGACACCACCCTCG
TGTCGCTCGACGCCAAGACCGGCAAGGTCAACTGGTCGGTCGTGAACGGCGATCCGAAGAAGGG
CGAGACCAACACCGCCACGGTTCTGCCCGTGAAGGACAAGGTCATCGTCGGCATCTCCGGCGGC
GAGTTCGGCGTGCAGTGCCACGTCACCGCCTACGACCTGAAGACCGGCAAGAAGGTGTGGCGCG
GCTACTCCGAGGGCCCGGACGATCAGATGATCGTGGACCCGGAGAAGACCACGTCGCTCGGCAA
GCCGATCGGCAAGGACTCCTCGCTGAAGACCTGGGAAGGCGATCAGTGGAAGACCGGCGGCGGC
TGCACCTGGGGCTGGTTCTCGTACGATCCGAAGCTCGACCTGATGTACTACGGCTCGGGCAACC
CCTCGACCTGGAACCCCAAGCAGCGTCCGGGCGACAACAAGTGGTCCATGACCATCTGGGCGCG
TAACCCGGATACCGGCATGGCCAAGTGGGTCTACCAGATGACCCCGCACGACGAGTGGGACTAC
GACGGCATCAACGAGATGATCCTCACGGATCAGAAGGTTGACGGCAAGGACCAGCCGCTCCTGA
CCCACTTCGACCGTAACGGCTTCGGCTACACGCTGAACCGCGAGACCGGCGCCCTGCTCGTCGC
CGAGAAGTTCGACCCGGCCGTCAACTGGGCGTCCAAGGTCGACATGGACAAGGGCTCGAAGAAC
TACGGCCGTCCGCTGGTCGTGTCGAAGTACTCGACCGAGCAGAACGGTGAGGACACCAACTCCA
AGGGCATCTGCCCGGCGGCGCTGGGCACCAAGGATCAGCAGCCTGCGGCCTTCTCGCCGAAGAC
CAACCTGTTCTACGTGCCCACCAACCACGTCTGCATGGACTACGAGCCGTTCCGGGTGACCTAC
ACCCCGGGCCAGCCCTACGTCGGTGCGACCCTCTCGATGTACCCGGCCCCGAACTCGCACGGCG
GCATGGGCAACTTCATCGCGTGGGATGGCGTCAACGGCAAGATCAAGTGGTCCAACCCCGAGCA
GTTCTCGGTGTGGTCCGGTGCTCTGGCCACCGCTGGCGACGTCGTGTTCTACGGCACGCTTGAG
GGCTACCTGAAGGCGGTCGACGACAAGACCGGCAAGGAGCTGTTCAAGTTCAAGACCCCGTCGG
GCATCATCGGTAACGTGATGACCTACCAGCACAAGGGCAAGCAGTACGTGGGCGTCCTGTCGGG
CGTCGGCGGCTGGGCTGGCATCGGCCTCGCGGCCGGCCTGACCGACCCGAACGCCGGCCTCGGC
GCGGTGGGTGGCTACGCGGCTCTGTCGCAGTACACCAACCTCGGCGGCCAGCTGACGGTCTTCG
CCCTGCCGAACTAA
-Exemplary Methylobacterium sp. XJLW Methanol dehydrogenase
(MDH-12) Amino Acid Sequence
SEQ ID NO: 194
MRAVHLLALGAGVAAVAAPALANESVMKGIANPAEQVLQTVDYANTRYSKLDQINAKNVKDLQV
AWTFSTGVLRGHEGSPLVVGNIMYVHTPFPNIVYALDLDHEAKIIWKYEPKQDPSVIPVMCCDT
VNRGLAYADGAILLHQADTTLVSLDAKTGKVNWSVVNGDPKKGETNTATVLPVKDKVIVGISGG
EFGVQCHVTAYDLKTGKKVWRGYSEGPDDQMIVDPEKTTSLGKPIGKDSSLKTWEGDQWKTGGG
CTWGWFSYDPKLDLMYYGSGNPSTWNPKQRPGDNKWSMTIWARNPDTGMAKWVYQMTPHDEWDY
DGINEMILTDQKVDGKDQPLLTHFDRNGFGYTLNRETGALLVAEKFDPAVNWASKVDMDKGSKN
YGRPLVVSKYSTEQNGEDTNSKGICPAALGTKDQQPAAFSPKTNLFYVPTNHVCMDYEPFRVTY
TPGQPYVGATLSMYPAPNSHGGMGNFIAWDGVNGKIKWSNPEQFSVWSGALATAGDVVFYGTLE
GYLKAVDDKTGKELFKFKTPSGIIGNVMTYQHKGKQYVGVLSGVGGWAGIGLAAGLTDPNAGLG
AVGGYAALSQYTNLGGQLTVFALPN
-Exemplary Methylobacterium sp. XJLW Aldehyde dehydrogenase
(ALDH-13) Nucleic Acid Coding Sequence
SEQ ID NO: 195
ATGAGAGCAATCGTCTATAATGGACCCCGCGATGTTTCGATGCAGGACGTGCCGGATGCGAAGA
TCGTGAAGCCGACCGACGTTCTGGTCCGCATCACGAGCACCAACATCTGCGGCTCCGACCTACA
TATGTACGAAGGCCGAACCGATTTTCCCCAAGGTGGCGTGTTCGGGCACGAGAACCTGGGACAG
GTGGCGGAAGTCGGCAGCGCCGTCGATCGGGTGCAGGTCGGGGACTGGGTCGCCGTCCCGTTCA
ACATCGGCTGCGGGTTCTGCGAAAACTGCGAGCGCGGCCTGAGCGCCTACTGCTTGACCACGGC
GGATCGAAGCGTCGTGCCGAACATGGCGGGCGCGGCCTACGGCTTTGCCGGCATGGGACCGTAT
CGCGGCGGTCAGGCCGATTTTCTGCGCGTCCCCTATGGCGACTATAACTGTCTGCAGCTGCCGC
CGGACGCGGAGGAGAGGCAGAACGACTATGTCATGCTGGCCGACATCTTTCCGACCGGCTGGCA
CTGCACGGAACTCGCAGGCGTGAAGCCCGGCGAAACCGTTGTGGTTTACGGGGCCGGGCCGGTC
GGTCTCATGGCCGCCTACTCGGCGATGATCAAGGGTGCGTCCCTGGTCATGGTTGTCGATCGCC
ATCCCGACCGGCTGCGCCTCGCCGAATCGATCGGTGCCGTGACCATCGACGATTCCAAGGACTC
CCCGGTGGACAAGGTGCTTGAGTTGACGAAGGGCGTCGGCGCCGACCGCGGCTGCGAGTGCGTC
GGCTACCAAGCGCACGACCCCAGCGGCCAGGAGCGCCCCAATATGACCATGAACGACTTGGTCA
AGTCGGTGAAATTCACCGGCGGCATCGGCGTGGTCGGCGTCTTCACGCCCCAGGATCCGGCCCC
GCAGGACCCGCTCTACAAGCAGGGCGAGATTGTGTTCGACCACGGCCTCTTCTGGTTCAAAGGT
CAGACGATCGGCGTCGGCCAGTGCAACGTGAAGGCCTATAACCGGCAGTTGCGCGACCTCATCT
CGACCGGCCGGGCGAAGCCGTCCTTCATCGTCTCGCACGAGCTTCCGCTGGGAGAGGCGCCGAA
GGCCTACAAGCACTTCGACGCGCGCGACGATGGCTGGACCAAGGTGATCCTCAAGCCCGCCGCC
TGA
-Exemplary Methylobacterium sp. XJLW Aldehyde dehydrogenase
(ALDH-13) Amino Acid Sequence
SEQ ID NO: 196
MRAIVYNGPRDVSMQDVPDAKIVKPTDVLVRITSTNICGSDLHMYEGRTDFPQGGVFGHENLGQ
VAEVGSAVDRVQVGDWVAVPFNIGCGFCENCERGLSAYCLTTADRSVVPNMAGAAYGFAGMGPY
RGGQADFLRVPYGDYNCLQLPPDAEERQNDYVMLADIFPTGWHCTELAGVKPGETVVVYGAGPV
GLMAAYSAMIKGASLVMVVDRHPDRLRLAESIGAVTIDDSKDSPVDKVLELTKGVGADRGCECV
GYQAHDPSGQERPNMTMNDLVKSVKFTGGIGVVGVFTPQDPAPQDPLYKQGEIVFDHGLFWFKG
QTIGVGQCNVKAYNRQLRDLISTGRAKPSFIVSHELPLGEAPKAYKHFDARDDGWTKVILKPAA
-Exemplary Methylobacterium sp. XJLW Aldehyde dehydrogenase
(ALDH-14) Nucleic Acid Coding Sequence
SEQ ID NO: 197
ATGTCCGGCACGTCGCACTCGCCCGCCGCCGACCGGGTCGCCGCCCTCCTGACCGACTTCCTGC
CGGGCGGCCGCATCGGCAGCGTCGTGGCCGGCGAGGTCCTCGCCGGGACCGGCGCCGCCCTCGA
CCTCGTCAACCCCGCGGACGGCGGCGTGCTCGCGACCTTCGCCGATGCCGGGCCGTCGGTGGTC
GAGGCCGCGATGGCGGCGGCCCGCGACGCCCAGCGCGCGTGGTGGGGGATGAGCGCCGCCGCCC
GGGGCCGGGCCCTGTGGGCGGTCGCCGCCCTGGTCCGGCAGCACGCCGGGGCGCTCGCTGAGCT
GGAGACCCTCTCGGCCGGCAAGCCGATCCGCGACACGCGCGGCGAGGTCGCCAAGGTCGCCGAG
ATGTTCGAGTATTATGCCGGCTGGTGCGACAAGCTTCACGGCGACGTCATCCCGGTGCCGAGTT
CGCACCTGAACTACACCCGCCACGAGCCCTTCGGCACCGTGGTGCAGATCACCCCCTGGAACGC
GCCGATCTTCACCGCCGGCTGGCAGATCGCCCCGGCCCTCTGCGCCGGCAACGCCGTGGTGCTG
AAGCCCTCCGAGCTGACACCGCTGACCTCGCTGGCGCTGGGCCTGCTCTGCGACCGCGCCGAGG
GGATGCCCCGCGGCCTCGTCTCGGTGCTGGCCGGCGCCGGTCCGACCACGGGGGCCGCCGCGGT
GGCCCATCCCGACACCCGCCTCGTCGTGTTCGTCGGCTCGGCCGAGGCCGGCGCGCAGATCGCC
GCCGCGGCGGCCCGCGCCATCGTGCCGAGCGTGCTGGAGCTCGGCGGCAAGTCGGCCAACATCG
TGTTCGCCGACGCCGACCTCGACCGGGCGCTGATCGGCGCGCAGGCCGCGATCTTCGGCGGCGC
CGGCCAGAGCTGCGTGGCGGGCTCCCGCCTCCTCGTGCACCGTTCGATCCACGCGTCCTTCGTG
GAGCGCCTGTCCCACGCCGCCGCGCGCATCCCGGTGGGGGCGCCGACCGACCCGGCGACGCAGA
TCGGGCCGATCAACAACCGGCGCCAGCGCGACAAGATCGCCGGCATGGTCGAGGCCGCGGCGAG
CGCCGGCGCCACCATCGCGGCCGGCGGGGCCTGCCCCGCGTCCCTGCGGGACACGGGCGGCTTC
TATTTCGGCCCGACCATCGTGGACGGCGTCGCGCCGGACGCGGCGATCGCCCGGGAGGAGGTGT
TCGGCCCGGTCCTCACGGTCCTGCCGTTCGACGGCGAGGACGAGGCGGTGGCGCTGGCCAACGG
CACGCCCTACGGCCTCGCGGGCGCGGTCTGGACCGGCGACGGCGGTCGCGGCCACCGGGTCGCG
GCGGCTTTGCGGGCCGGAACGGTGTGGGTCAACGGCTACAAGACCATCAACGTGGCCTCGCCGT
TCGGCGGCTTCGGCCGCTCGGGCTTCGGCCGCTCCTCGGGCCGCGAGGCGCTGATGGCCTACAC
GCAGACCAAGAGCGTCTGGGTCGAGACCGCGGCCCAGCCGGCGGTGACCTTCGGCTACGTGGGC
TAG
-Exemplary Methylobacterium sp. XJLW Aldehyde dehydrogenase
(ALDH-14) Amino Acid Sequence
SEQ ID NO: 198
MSGTSHSPAADRVAALLTDFLPGGRIGSVVAGEVLAGTGAALDLVNPADGGVLATFADAGPSVV
EAAMAAARDAQRAWWGMSAAARGRALWAVAALVRQHAGALAELETLSAGKPIRDTRGEVAKVAE
MFEYYAGWCDKLHGDVIPVPSSHLNYTRHEPFGTVVQITPWNAPIFTAGWQIAPALCAGNAVVL
KPSELTPLTSLALGLLCDRAEGMPRGLVSVLAGAGPTTGAAAVAHPDTRLVVFVGSAEAGAQIA
AAAARAIVPSVLELGGKSANIVFADADLDRALIGAQAAIFGGAGQSCVAGSRLLVHRSIHASFV
ERLSHAAARIPVGAPTDPATQIGPINNRRQRDKIAGMVEAAASAGATIAAGGACPASLRDTGGF
YFGPTIVDGVAPDAAIAREEVFGPVLTVLPFDGEDEAVALANGTPYGLAGAVWTGDGGRGHRVA
AALRAGTVWVNGYKTINVASPFGGFGRSGFGRSSGREALMAYTQTKSVWVETAAQPAVTFGYVG

### D) Xylulose Monophosphate Pathway

In some embodiments, compositions and methods described herein comprise introduction of one or more genes coding for dihydroxyacetone synthase (DAS), Formolase and/or dihydroxyacetone kinase (DAK). In some embodiments, these enzymes metabolize the substrates HCHO and/or D-xylulose 5-phosphate (Xu5P) to produce dihydroxyacetone (DHA), glyceraldehyde 3-phosphate (3PGA) Glycoaldehyde (GALD) and/or dihydroxyacetone phosphate (DHAP), a component that can be incorporated into the Calvin-Benson cycle, a photosynthetic carbon fixation pathway. In some embodiments, genes are introduced that comprise coding sequences for DAS-like and/or DAK-like proteins. In some embodiments, DAS and DAK function are incorporated into one enzyme, and only one gene is introduced that facilitates the conversion of formaldehyde and/or D-xylulose 5-phosphate (Xu5P) directly to glyceraldehyde 3-phosphate (3PGA) and DHAP.

### Dihydroxyacetone Synthase (DAS) and DAS-Like

In certain embodiments, a composition described herein comprises at least one transgenic DAS and/or DAS-like enzyme. In certain embodiments, DAS and/or DAS like proteins utilize Formaldehyde with D-xylulose 5-phosphate as a substrate and produce D-glyceraldehyde 3-phosphate and dihydroxyacetone.

In some embodiments, a DAS and/or DAS-like gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 200, 202, 204, or 206 (or a portion thereof). In some embodiments, a DAS and/or DAS-like gene and/or transgene comprises a nucleotide sequence that is at least 90%, 91%, 92%, 93%, 94%, 95%, 96%, 97%, 98%, 99%, or 100% identical to any one of SEQ ID NOs: 199, 201, 203, or 205 (or a portion thereof).

-Exemplary Candida boidinii Dihydroxyacetone synthase (DASCanbo)
Nucleic Acid Coding Sequence
SEQ ID NO: 199
ATGGCTTTAGCTAAGGCTGCTTCTATAAATGATGACATCCACGATCTTACAATGAGAGCGTTCA
GATGCTACGTCCTTGACCTTGTCGAGCAATATGAGGGCGGTCACCCAGGTTCTGCCATGGGTAT
GGTCGCGATGGGTATCGCCCTATGGAAATACACTATGAAATACAGCACTAATGACCCAACGTGG
TTCAACAGGGATAGATTTGTATTATCCAACGGTCACGTCTGTCTTTTCCAATATCTCTTTCAGC
ACTTGAGTGGCTTAAAATCAATGACTGAGAAGCAGTTAAAGAGTTACCACTCTAGTGATTATCA
CTCAAAGTGTCCGGGACATCCGGAAATCGAGAATGAGGCCGTAGAGGTGACTACAGGCCCTCTT
GGTCAGGGCATATCGAATTCAGTTGGTCTGGCCATCGCCTCAAAGAATCTTGGTGCACTTTATA
ACAAACCTGGCTATGAAGTGGTAAACAACACCACATACTGCATTGTAGGCGATGCATGCCTTCA
AGAGGGGCCAGCCCTTGAGTCCATATCCTTCGCAGGGCACCTCGGACTCGACAATCTCGTCGTT
ATCTATGACAATAACCAAGTGTGTTGTGACGGTTCTGTGGATATTGCCAACACTGAGGATATTT
CAGCAAAGTTTCGAGCTTGTAATTGGAACGTGATCGAGGTCGAGGACGGCGCAAGGGATGTTGC
TACGATTGTTAAGGCTTTGGAGTTAGCAGGGGCCGAGAAGAACCGGCCAACTCTTATCAACGTG
CGGACGATAATTGGTACTGACTCAGCCTTTCAGAATCACTGCGCCGCGCATGGTTCTGCTCTGG
GTGAGGAAGGAATTCGTGAACTAAAGATAAAATACGGTTTCAATCCGAGCCAGAAATTCCATTT
TCCCCAGGAAGTATACGATTTCTTCTCGGACATTCCTGCAAAAGGTGACGAATACGTCTCCAAT
TGGAACAAGCTAGTGAGCTCATATGTTAAAGAGTTTCCAGAATTGGGCGCAGAATTCCAGTCTA
GGGTCAAGGGAGAACTTCCCAAGAACTGGAAATCTTTATTACCGAACAACTTGCCTAATGAGGA
CACTGCTACTCGAACAAGTGCACGTGCGATGGTGCGTGCGCTCGCTAAAGATGTGCCTAATGTG
ATCGCGGGGTCCGCGGACCTCTCCGTTTCAGTCAATCTACCTTGGCCGGGTAGCAAATATTTTG
AGAATCCACAATTAGCAACTCAGTGCGGACTAGCAGGTGACTATTCCGGAAGATACGTGGAATT
CGGTATAAGGGAACACTGTATGTGCGCGATCGCCAACGGGCTTGCTGCGTTCAACAAAGGTACT
TTCTTGCCAATAACTTCATCGTTCTACATGTTCTATCTCTATGCAGCTCCGGCCCTTAGGATGG
CTGCACTTCAAGAGCTCAAGGCCATTCACATCGCTACTCACGACTCTATCGGAGCTGGAGAGGA
CGGCCCAACGCACCAACCCATTGCTCAAAGCGCGCTTTGGCGAGCTATGCCAAACTTTTACTAC
ATGAGGCCCGGGGATGCAAGCGAGGTACGGGGACTCTTTGAGAAAGCAGTTGAATTGCCCTTAA
GTACCCTGTTCAGTTTAAGTCGGCACGAAGTGCCACAATACCCTGGCAAGAGCTCGATCGAGTT
GGCCAAGAGAGGCGGCTATGTGTTCGAAGATGCTAAAGATGCTGATATACAGCTTATCGGTGCG
GGAAGCGAACTCGAACAGGCCGTTAAAACTGCTCGAATACTCCGATCGAGAGGTCTTAAAGTCC
GTATCCTTAGCTTCCCATGTCAGCGTTTATTTGACGAGCAATCGGTGGGATACCGTAGAAGTGT
TCTTCAAAGAGGTAAGGTCCCGACTGTGGTGATCGAGGCATATGTTGCGTATGGATGGGAGAGA
TACGCTACTGCAGGTTATACTATGAACACGTTCGGAAAGTCCCTGCCGGTAGAGGATGTGTATG
AGTACTTTGGTTTCAATCCATCCGAAATCAGCAAGAAAATTGAGGGATATGTGAGAGCCGTCAA
AGCCAATCCAGATTTGCTCTACGAATTTATCGATCTCACAGAGAAGCCTAAACACGATCAAAAT
CACCTTTAA
-Exemplary Candida boidinii Dihydroxyacetone synthase (DASCanbo)
Amino Acid Sequence
SEQ ID NO: 200
MALAKAASINDDIHDLTMRAFRCYVLDLVEQYEGGHPGSAMGMVAMGIALWKYTMKYSTNDPTW
FNRDRFVLSNGHVCLFQYLFQHLSGLKSMTEKQLKSYHSSDYHSKCPGHPEIENEAVEVTTGPL
GQGISNSVGLAIASKNLGALYNKPGYEVVNNTTYCIVGDACLQEGPALESISFAGHLGLDNLVV
IYDNNQVCCDGSVDIANTEDISAKFRACNWNVIEVEDGARDVATIVKALELAGAEKNRPTLINV
RTIIGTDSAFQNHCAAHGSALGEEGIRELKIKYGFNPSQKFHFPQEVYDFFSDIPAKGDEYVSN
WNKLVSSYVKEFPELGAEFQSRVKGELPKNWKSLLPNNLPNEDTATRTSARAMVRALAKDVPNV
IAGSADLSVSVNLPWPGSKYFENPQLATQCGLAGDYSGRYVEFGIREHCMCAIANGLAAFNKGT
FLPITSSFYMFYLYAAPALRMAALQELKAIHIATHDSIGAGEDGPTHQPIAQSALWRAMPNFYY
MRPGDASEVRGLFEKAVELPLSTLFSLSRHEVPQYPGKSSIELAKRGGYVFEDAKDADIQLIGA
GSELEQAVKTARILRSRGLKVRILSFPCQRLFDEQSVGYRRSVLQRGKVPTVVIEAYVAYGWER
YATAGYTMNTFGKSLPVEDVYEYFGFNPSEISKKIEGYVRAVKANPDLLYEFIDLTEKPKHDQN
HL
-Exemplary Synthetic Formolase (Formolase) Nucleic Acid Coding
Sequence
SEQ ID NO: 201
ATGGCTATGATAACTGGTGGTGAACTTGTTGTGAGAACCCTGATTAAGGCCGGAGTAGAACACC
TGTTTGGGTTGCACGGAATCCATATCGACACAATTTTCCAGGCGTGTTTGGACCACGACGTTCC
TATCATTGACACAAGACACGAAGCCGCCGCGGGCCATGCTGCCGAAGGATATGCCAGAGCAGGT
GCTAAGTTAGGGGTCGCGCTGGTGACCGCAGGTGGTGGATTCACTAACGCGGTTACGCCAATTG
CCAACGCCAGGACAGACAGGACCCCAGTTTTGTTCTTGACCGGTAGCGGTGCTTTAAGAGACGA
CGAAACCAATACTCTTCAGGCAGGTATCGACCAGGTTGCAATGGCGGCCCCTATAACTAAGTGG
GCTCATAGAGTTATGGCGACCGAACATATACCGAGGCTCGTGATGCAGGCAATCAGGGCTGCTT
TATCCGCTCCTCGTGGACCTGTGCTGTTGGACCTTCCTTGGGATATCCTCATGAACCAAATAGA
CGAAGATTCAGTTATAATTCCTGACTTGGTCCTCTCCGCACACGGAGCACATCCCGATCCTGCG
GATCTTGACCAGGCGCTCGCACTCCTCAGGAAAGCCGAAAGACCAGTAATTGTGCTGGGCTCAG
AGGCCTCTCGAACAGCTCGTAAAACAGCATTATCAGCTTTCGTCGCCGCCACCGGAGTCCCAGT
GTTTGCAGACTACGAGGGACTAAGTATGCTATCTGGGCTGCCTGACGCTATGAGGGGTGGCCTT
GTCCAGAATTTATATAGCTTTGCCAAGGCTGACGCAGCACCCGATCTTGTTCTTATGTTGGGTG
CTCGTTTCGGTCTTAATACAGGTCACGGTTCAGGTCAATTGATTCCACATAGTGCTCAGGTCAT
ACAAGTCGACCCGGATGCTTGCGAGCTAGGCAGACTCCAAGGAATCGCTCTCGGAATAGTTGCC
GACGTTGGTGGGACAATAGAAGCGCTAGCACAAGCAACAGCACAAGACGCCGCCTGGCCAGATC
GTGGTGACTGGTGCGCAAAGGTGACTGACCTGGCCCAAGAACGTTATGCCAGCATCGCCGCGAA
GTCCTCATCAGAGCACGCTCTCCACCCATTCCATGCTTCGCAGGTGATAGCTAAACACGTTGAC
GCTGGTGTTACAGTCGTTGCGGACGGCGGACTAACTTACCTTTGGCTTTCAGAGGTAATGTCAA
GGGTAAAGCCAGGTGGATTCCTCTGCCACGGCTATCTTAACAGCATGGGTGTCGGTTTCGGAAC
TGCGCTCGGCGCCCAGGTAGCAGACCTCGAAGCGGGAAGAAGAACGATACTCGTTACTGGGGAC
GGATCAGTTGGCTACAGTATAGGTGAATTTGACACTCTCGTACGAAAACAATTGCCACTTATTG
TTATTATAATGAACAACCAATCTTGGGGCTGGACTTTGCACTTCCAGCAATTAGCAGTCGGACC
AAACAGGGTTACAGGTACTAGACTTGAGAATGGGTCCTACCATGGGGTGGCTGCAGCTTTTGGG
GCCGACGGATATCACGTGGACTCGGTTGAATCATTCAGCGCTGCTTTGGCACAGGCCCTGGCAC
ATAACAGGCCTGCATGCATTAACGTTGCAGTGGCTCTCGACCCAATTCCGCCTGAGGAGCTGAT
ACTCATTGGCATGGATCCTTTCGCCTGA
-Exemplary Synthetic Formolase (Formolase) Amino Acid Sequence
SEQ ID NO: 202
MAMITGGELVVRTLIKAGVEHLFGLHGIHIDTIFQACLDHDVPIIDTRHEAAAGHAAEGYARAG
AKLGVALVTAGGGFTNAVTPIANARTDRTPVLFLTGSGALRDDETNTLQAGIDQVAMAAPITKW
AHRVMATEHIPRLVMQAIRAALSAPRGPVLLDLPWDILMNQIDEDSVIIPDLVLSAHGAHPDPA
DLDQALALLRKAERPVIVLGSEASRTARKTALSAFVAATGVPVFADYEGLSMLSGLPDAMRGGL
VQNLYSFAKADAAPDLVLMLGARFGLNTGHGSGQLIPHSAQVIQVDPDACELGRLQGIALGIVA
DVGGTIEALAQATAQDAAWPDRGDWCAKVTDLAQERYASIAAKSSSEHALHPFHASQVIAKHVD
AGVTVVADGGLTYLWLSEVMSRVKPGGFLCHGYLNSMGVGFGTALGAQVADLEAGRRTILVTGD
GSVGYSIGEFDTLVRKQLPLIVIIMNNQSWGWTLHFQQLAVGPNRVTGTRLENGSYHGVAAAFG
ADGYHVDSVESFSAALAQALAHNRPACINVAVALDPIPPEELILIGMDPFA
-Exemplary Pseudomonas fluorescens Benzaldehyde lyase (BAL) Nucleic
Acid Coding Sequence
SEQ ID NO: 203
ATGGCGATGATTACAGGCGGCGAACTGGTTGTTCGCACCCTAATAAAGGCTGGGGTCGAACATC
TGTTCGGCCTGCACGGCGCGCATATCGATACGATTTTTCAAGCCTGTCTCGATCATGATGTGCC
GATCATCGACACCCGCCATGAGGCCGCCGCAGGGCATGCGGCCGAGGGCTATGCCCGCGCTGGC
GCCAAGCTGGGCGTGGCTGGTCACGGCGGGCGGGGGATTTACCAATGCGGTCACGCCCATTGCC
AACGCTTGGCTGGATCGCAAGGCCGGTGTATTCCTCACCCGGGATCGGGCGCGCTGCGTGATGA
TGAAACCAACACGTTGCAGGCGGGGATTGATCAGGTCGCCATGGCGGCGCCCATTACCAAATGG
GCGCATCGGGTGATGGCAACCGAGCATATCCCACGGCTGGTGATGCAGGCGATCCGCGCCGCGT
TGAGCGCGCCACGCGGGCCGGTGTTGCTGGATCTGCCGTGGGATATTCTGATGAACCAGATTGA
TGAGGATAGCGTCATTATCCCCGATCTGGTCTTGTCCGCGCATGGGGCCAGACCCGACCCTGCC
GATCTGGATCAGGCTCTCGCGCTTTTGCGCAAGGCGGAGCGGCCGGTCATCGTGCTCGGCTCAG
AAGCCTCGCGGACAGCGCGCAAGACGGCGCTTAGCGCCTTCGTGGCGGCGACTGGCGTGCCGGT
GTTTGCCGATTATGAAGGGCTAAGCATGCTCTCGGGGCTGCCCGATGCTATGCGGGGGGGGCTG
GTGCAAAACCTCTATTCTTTTGCCAAAGCCGATGCCGCGCCAGATCTCGTGCTGATGCTGGGGG
CGCGCTTTGGCCTTAACACCGGGCATGGATCTGGGCAGTTGATCCCCCATAGCGCGCAGGTCAT
TCAGGTCGACCCTGATGCCTGCGAGCTGGGACGCCTGCAGGGCATCGCTCTGGGCATTGTGGCC
GATGTGGGTGGGACCATCGAGGCTTTGGCGCAGGCCACCGCGCAAGATGCGGCTTGGCCGGATC
GCGGCGACTGGTGCGCCAAAGTGACGGATCTGGCGCAAGAGCGCTATGCCAGCATCGCTGCGAA
ATCGAGCAGCGAGCATGCGCTCCACCCCTTTCACGCCTCGCAGGTCATTGCCAAACACGTCGAT
GCAGGGGTGACGGTGGTAGCGGATGGTGCGCTGACCTATCTCTGGCTGTCCGAAGTGATGAGCC
GCGTGAAACCCGGCGGTTTTCTCTGCCACGGCTATCTAGGCTCGATGGGCGTGGGCTTCGGCAC
GGCGCTGGGCGCGCAAGTGGCCGATCTTGAAGCAGGCCGCCGCACGATCCTTGTGACCGGCGAT
GGCTCGGTGGGCTATAGCATCGGTGAATTTGATACGCTGGTGCGCAAACAATTGCCGCTGATCG
TCATCATCATGAACAACCAAAGCTGGGGGGCGACATTGCATTTCCAGCAATTGGCCGTCGGCCC
CAATCGCGTGACGGGCACCCGTTTGGAAAATGGCTCCTATCACGGGGTGGCCGCCGCCTTTGGC
GCGGATGGCTATCATGTCGACAGTGTGGAGAGCTTTTCTGCGGCTCTGGCCCAAGCGCTCGCCC
ATAATCGCCCCGCCTGCATCAATGTCGCGGTCGCGCTCGATCCGATCCCGCCCGAAGAACTCAT
TCTGATCGGCATGGACCCCTTCGCATGA
-Exemplary Pseudomonas fluorescens Benzaldehyde lyase (BAL) Amino
Acid Sequence
SEQ ID NO: 204
MAMITGGELVVRTLIKAGVEHLFGLHGAHIDTIFQACLDHDVPIIDTRHEAAAGHAAEGYARAG
AKLGVAGHGGRGIYQCGHAHCQRLAGSQGRCIPHPGSGALRDDETNTLQAGIDQVAMAAPITKW
AHRVMATEHIPRLVMQAIRAALSAPRGPVLLDLPWDILMNQIDEDSVIIPDLVLSAHGARPDPA
DLDQALALLRKAERPVIVLGSEASRTARKTALSAFVAATGVPVFADYEGLSMLSGLPDAMRGGL
VQNLYSFAKADAAPDLVLMLGARFGLNTGHGSGQLIPHSAQVIQVDPDACELGRLQGIALGIVA
DVGGTIEALAQATAQDAAWPDRGDWCAKVTDLAQERYASIAAKSSSEHALHPFHASQVIAKHVD
AGVTVVADGALTYLWLSEVMSRVKPGGFLCHGYLGSMGVGFGTALGAQVADLEAGRRTILVTGD
GSVGYSIGEFDTLVRKQLPLIVIIMNNQSWGATLHFQQLAVGPNRVTGTRLENGSYHGVAAAFG
ADGYHVDSVESFSAALAQALAHNRPACINVAVALDPIPPEELILIGMDPFA
-Exemplary Ogataea polymorpha Dihydroxyacetone synthase (DASOP)
Nucleic Acid Coding Sequence
SEQ ID NO: 205
ATGAGTATGAGAATCCCTAAAGCAGCGTCGGTCAACGACGAACAACACCAGAGAATCATCAAGT
ACGGTCGTGCTCTTGTCCTGGACATTGTCGAGCAGTACGGAGGAGGCCACCCGGGCTCGGCCAT
GGGCGCCATGGCTATCGGAATTGCTCTGTGGAAATACACCCTGAAATATGCTCCCAACGACCCT
AACTACTTCAACAGAGACAGGTTTGTCCTGTCGAACGGTCACGTGTGTCTGTTCCAGTATATCT
TCCAGCACCTGTACGGTCTCAAGTCGATGACCATGGCGCAGCTGAAGTCCTACCACTCGAATGA
CTTCCACTCGCTGTGTCCCGGTCACCCAGAAATCGAGCACGACGCCGTCGAGGTCACAACGGGC
CCGCTCGGCCAGGGTATCTCGAACTCTGTTGGTCTGGCCATAGCCACCAAAAACCTGGCTGCCA
CGTACAACAAGCCGGGCTTTGATATCATCACCAACAAGGTGTACTGCATGGTTGGCGATGCGTG
CTTGCAGGAGGGCCCTGCTCTCGAGTCGATCTCGCTGGCCGGCCACATGGGGCTGGACAATCTG
ATTGTGCTCTACGACAACAACCAGGTCTGCTGTGACGGCAGTGTTGACATTGCCAACACGGAGG
ACATCAGTGCCAAGTTCAAGGCCTGCAACTGGAACGTGATCGAGGTCGAGAACGCTTCCGAGGA
CGTGGCTACCATTGTCAAGGCCTTGGAGTACGCGCAGGCCGAGAAGCACAGACCAACACTTATC
AACTGCAGAACTGTGATTGGATCGGGTGCTGCGTTCGAGAACCACTGTGCTGCGCACGGTAACG
CTCTGGGCGAGGACGGTGTGCGCGAGCTCAAAATCAAGTACGGCATGAACCCGGCCCAGAAGTT
CTACATTCCGCAGGACGTGTACGACTTCTTCAAGGAGAAGCCGGCCGAGGGCGACAAGCTGGTG
GCCGAATGGAAGAGTCTCGTGGCCAAGTACGTCAAGGCGTACCCTGAGGAGGGCCAGGAGTTTT
TGGCGCGGATGAGAGGCGAGCTGCCAAAGAACTGGAAGTCGTTCCTGCCGCAGCAGGAATTCAC
CGGCGACGCTCCTACAAGGGCCGCTGCCAGAGAGCTTGTGAGAGCCCTGGGGCAGAACTGCAAG
TCGGTGATTGCCGGTTGCGCAGACCTGTCTGTGTCTGTCAATTTGCAGTGGCCAGGGGTGAAAT
ATTTCATGGACCCCTCGCTGTCCACGCAGTGTGGCCTGAGCGGCGACTACTCCGGCAGATACAT
TGAGTACGGAATCAGAGAACACGCCATGTGTGCTATCGCCAATGGCCTTGCCGCCTACAACAAG
GGCACGTTCCTGCCGATCACGTCGACTTTCTTCATGTTCTACCTGTACGCTGCCCCAGCCATCA
GAATGGCCGGCCTGCAGGAGCTCAAGGCGATCCACATCGGCACCCACGACTCGATCAATGAGGG
TGAGAACGGCCCTACGCACCAGCCGGTCGAGTCGCCAGCATTGTTCCGGGCCATGCCAAACATT
TACTACATGAGACCGGTCGACTCTGCAGAAGTGTTTGGCCTGTTCCAAAAAGCCGTCGAGCTGC
CATTCAGCTCGATTCTGTCGCTCTCGAGAAACGAGGTGCTGCAATACCCTGGCAAGTCGAGCGC
AGAGAAGGCGCAACGCGGCGGCTATATTCTGGAGGATGCGGAGAACGCCGAGGTGCAGATTATT
GGAGTTGGTGCAGAGATGGAGTTTGCATACAAGGCCGCCAAGATCTTGGGCAGAAAGTTCAGGA
CCAGAGTTCTCTCCATCCCATGCACGCGGCTGTTTGACGAGCAGTCGATCGGCTATAGACGCTC
GGTTTTGAGAAAGGACGGCAGACAGGTGCCAACGGTGGTGGTGGACGGCCACGTTGCGTTCGGC
TGGGAGAGATACGCTACGGCGTCCTACTGTATGAACACGTACGGCAAGTCTCTGCCTCCAGAAG
TGATCTACGAGTACTTTGGATACAACCCGGCAACGATTGCCAAGAAGGTCGAAGCGTACGTCCG
GGCGTGCCAAAGAGACCCTTTGCTGCTCCACGACTTCCTGGACCTGAAGGAAAAGCCTAACCAC
GATAAAGTAAATAAGCTCTGA
-Exemplary Ogataea polymorpha Dihydroxyacetone synthase (DASOP)
Amino Acid Sequence
SEQ ID NO: 206
MSMRIPKAASVNDEQHQRIIKYGRALVLDIVEQYGGGHPGSAMGAMAIGIALWKYTLKYAPNDP
NYFNRDRFVLSNGHVCLFQYIFQHLYGLKSMTMAQLKSYHSNDFHSLCPGHPEIEHDAVEVTTG
PLGQGISNSVGLAIATKNLAATYNKPGFDIITNKVYCMVGDACLQEGPALESISLAGHMGLDNL
IVLYDNNQVCCDGSVDIANTEDISAKFKACNWNVIEVENASEDVATIVKALEYAQAEKHRPTLI
NCRTVIGSGAAFENHCAAHGNALGEDGVRELKIKYGMNPAQKFYIPQDVYDFFKEKPAEGDKLV
AEWKSLVAKYVKAYPEEGQEFLARMRGELPKNWKSFLPQQEFTGDAPTRAAARELVRALGQNCK
SVIAGCADLSVSVNLQWPGVKYFMDPSLSTQCGLSGDYSGRYIEYGIREHAMCAIANGLAAYNK
GTFLPITSTFFMFYLYAAPAIRMAGLQELKAIHIGTHDSINEGENGPTHQPVESPALFRAMPNI
YYMRPVDSAEVFGLFQKAVELPFSSILSLSRNEVLQYPGKSSAEKAQRGGYILEDAENAEVQII
GVGAEMEFAYKAAKILGRKFRTRVLSIPCTRLFDEQSIGYRRSVLRKDGRQVPTVVVDGHVAFG
WERYATASYCMNTYGKSLPPEVIYEYFGYNPATIAKKVEAYVRACQRDPLLLHDFLDLKEKPNH
DKVNKL

### Dihydroxyacetone Kinase (DAK)

In certain embodiments, a composition described herein comprises at least one transgenic DAK and/or DAK-like enzyme. In certain embodiments, DAK and/or DAK-like proteins utilize dihydroxyacetone as a substrate and produce dihydroxyacetone-phosphate.

In some embodiments, a DAK and/or DAK-like gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 208, 210, 212, or 214 (or a portion thereof). In some embodiments, a DAK and/or DAK-like gene and/or transgene comprises a nucleotide sequence that is at least 90%, 91%, 92%, 93%, 94%, 95%, 96%, 97%, 98%, 99%, or 100% identical to any one of SEQ ID NOs: 207, 209, 211, or 213 (or a portion thereof).

-Exemplary Saccharomyces cerevisiae S288C Dihydroxyacetone Kinase
(DAKY) Nucleic Acid Coding Sequence
SEQ ID NO: 207
ATGTCCCATAAGCAATTCAAGAGCGACGGTAACATCGTTACACCTTACCTTCTAGGATTAGCTA
GAAGTAACCCTGGCCTCACCGTGATCAAACACGACAGAGTCGTCTTTCGTACGGCAAGTGCTCC
CAATTCTGGTAATCCACCTAAAGTCAGTTTGGTTTCTGGTGGTGGGAGTGGCCATGAGCCGACT
CACGCCGGATTCGTTGGAGAAGGTGCTCTCGATGCTATTGCCGCTGGTGCAATATTCGCATCTC
CTAGTACAAAGCAAATCTACAGTGCCATCAAAGCCGTTGAATCTCCAAAAGGTACCCTTATTAT
AGTGAAGAATTATACGGGAGACATTATTCATTTTGGACTAGCAGCGGAAAGAGCTAAAGCGGCT
GGTATGAAGGTTGAACTTGTCGCAGTCGGGGACGACGTATCAGTTGGCAAGAAGAAGGGATCGC
TAGTCGGCCGACGTGGGCTGGGAGCGACGGTGCTTGTACACAAAATAGCTGGGGCTGCCGCGTC
TCACGGATTGGAGCTCGCTGAGGTCGCAGAAGTGGCCCAAAGTGTAGTTGATAACTCTGTAACC
ATCGCGGCGTCTCTGGACCATTGTACGGTACCTGGTCACAAACCAGAAGCTATCCTAGGTGAGA
ATGAGTACGAAATAGGAATGGGAATACATAACGAGAGTGGAACATATAAGTCCAGCCCACTTCC
AAGCATCTCCGAGCTAGTATCCCAAATGCTCCCATTGTTGTTAGATGAGGACGAGGACAGGAGC
TACGTGAAGTTTGAGCCCAAAGAGGATGTGGTCTTGATGGTTAACAACATGGGCGGCATGTCCA
ACCTCGAATTAGGGTATGCTGCCGAAGTCATTTCTGAGCAATTAATCGACAAATATCAGATAGT
CCCTAAGCGGACCATCACCGGGGCGTTCATTACAGCTCTCAATGGTCCCGGTTTTGGGATAACA
CTAATGAATGCATCCAAGGCTGGTGGTGATATACTCAAATATTTCGACTACCCCACTACAGCTA
GTGGATGGAACCAGATGTATCACTCGGCAAAAGACTGGGAAGTTCTTGCAAAGGGACAAGTACC
CACTGCTCCAAGTTTGAAAACATTAAGAAACGAGAAAGGATCAGGCGTGAAAGCTGACTATGAC
ACCTTCGCCAAAATTTTACTCGCTGGTATAGCAAAGATTAATGAAGTTGAGCCTAAGGTCACCT
GGTATGACACTATTGCAGGGGACGGTGACTGTGGCACCACGCTTGTTAGCGGTGGAGAAGCGTT
AGAGGAAGCTATCAAGAACCACACCTTAAGGCTTGAGGACGCAGCTTTGGGAATCGAAGATATA
GCCTACATGGTTGAGGACTCAATGGGCGGCACTTCAGGTGGGCTCTATTCCATTTATCTATCCG
CATTGGCTCAAGGTGTTAGAGACTCAGGCGACAAAGAGTTGACAGCGGAGACTTTCAAGAAGGC
TTCAAATGTAGCACTAGACGCTCTCTACAAATATACCAGAGCGCGACCAGGCTACCGTACGTTA
ATCGATGCCTTACAACCGTTCGTTGAAGCCCTTAAGGCTGGTAAAGGTCCTCGGGCTGCTGCAC
AAGCAGCATATGATGGGGCAGAAAAGACCAGGAAGATGGACGCGTTAGTCGGGCGTGCCTCTTA
TGTGGCTAAAGAGGAGTTGCGTAAGCTTGATAGTGAGGGTGGACTCCCAGATCCTGGAGCCGTG
GGACTTGCAGCACTTCTCGATGGATTTGTGACAGCGGCAGGCTATTAG
-Exemplary Saccharomyces cerevisiae S288C Dihydroxyacetone Kinase
(DAKY) Amino Acid Sequence
SEQ ID NO: 208
MSHKQFKSDGNIVTPYLLGLARSNPGLTVIKHDRVVFRTASAPNSGNPPKVSLVSGGGSGHEPT
HAGFVGEGALDAIAAGAIFASPSTKQIYSAIKAVESPKGTLIIVKNYTGDIIHFGLAAERAKAA
GMKVELVAVGDDVSVGKKKGSLVGRRGLGATVLVHKIAGAAASHGLELAEVAEVAQSVVDNSVT
IAASLDHCTVPGHKPEAILGENEYEIGMGIHNESGTYKSSPLPSISELVSQMLPLLLDEDEDRS
YVKFEPKEDVVLMVNNMGGMSNLELGYAAEVISEQLIDKYQIVPKRTITGAFITALNGPGFGIT
LMNASKAGGDILKYFDYPTTASGWNQMYHSAKDWEVLAKGQVPTAPSLKTLRNEKGSGVKADYD
TFAKILLAGIAKINEVEPKVTWYDTIAGDGDCGTTLVSGGEALEEAIKNHTLRLEDAALGIEDI
AYMVEDSMGGTSGGLYSIYLSALAQGVRDSGDKELTAETFKKASNVALDALYKYTRARPGYRTL
IDALQPFVEALKAGKGPRAAAQAAYDGAEKTRKMDALVGRASYVAKEELRKLDSEGGLPDPGAV
GLAALLDGFVTAAGY
-Exemplary Komagataella phaffii GS115 (Pischia pastoris)
Dihydroxyacetone Kinase (DAKP) Nucleic Acid Coding Sequence
SEQ ID NO: 209
ATGAGTTCAAAACATTGGGATTACAAGAAGGACCTTGTTCTTAGTCACCTGGCGGGTTTATGCC
AGTCCAACCCACATGTTAGGCTGATCGAATCCGAGAGGGTGGTAATCTCCGCTGAAAATCAGGA
AGATAAGATAACATTGATCAGTGGTGGTGGTTCAGGCCATGAGCCTTTACATGCCGGTTTCGTG
ACCAAGGACGGACTTTTAGACGCCGCTGTGGCGGGTTTCATTTTCGCCTCTCCCAGCACTAAGC
AGATATTCTCTGCAATCAAAGCGAAACCTTCTAAGAAAGGAACACTGATCATCGTGAAGAACTA
CACTGGGGACATATTGCATTTTGGCCTAGCAGCCGAGAAAGCGAAAGCTGAAGGGCTTAATGCG
GAACTCCTCATCGTCCAAGACGATGTGAGCGTTGGCAAGGCTAAGAACGGGCTTGTCGGTAGAA
GAGGTTTGGCTGGTACCTCACTGGTTCACAAGATTCTAGGGGCCAAAGCTTACTTACAAAAGGA
TAACTTGGAGTTGCACCAGCTAGTTACATTTGGTGAGAAAGTTGTCGCTAACCTCGTAACGATC
GGAGCGAGTCTTGACCATGTCACAATTCCAGCCCGAGCTAACAAGCAGGAAGAGGACGACTCTG
ACGATGAGCATGGGTACGAAGTACTAAAACACGACGAATTTGAGATTGGTATGGGTATACATAA
TGAGCCCGGTATTAAGAAATCATCACCCATACCCACCGTTGACGAACTTGTCGCGGAATTGCTC
GAATATCTACTTTCTACCACAGACAAAGATAGGAATTACGTTCAATTCGATAAGAACGATGAGG
TGGTGTTGCTTATCAACAACCTGGGCGGGACATCTGTGCTTGAGCTCTACGCTATCCAGAATAT
CGTTGTTGACCAATTGGCGTCCAAATACTCTATCAAGCCAGTGAGAATATTTACAGGCACCTTT
ACTACCTCTTTGGACGGACCAGGATTTTCAATTACGCTTTTGAACGCTACAAAGACAGGAGACA
AGGACATCTTGAAGTTTCTCGATCATAAAACGTCCGCACCTGGATGGAACTCTAACATCTCGGA
CTGGTCCGGTAGAGTAGACAATTTCATAGTAGCCGCGCCAGAAATCGATGAGGGAGATAGCTCT
AGTAAAGTTTCTGTGGATGCTAAGCTTTATGCGGACCTGCTTGAGTCCGGTGTGAAGAAAGTGA
TTTCAAAAGAACCCAAAATCACTCTCTACGATACCGTTGCTGGAGATGGTGACTGTGGAGAAAC
ATTGGCAAACGGGAGTAACGCTATACTAAAAGCTTTAGCTGAGGGGAAATTGGATCTCAAGGAC
GGGGTCAAGTCCCTTGTACAGATTACCGACATAGTGGAAACAGCGATGGGCGGGACTTCCGGTG
GCCTTTACTCAATTTTCATAAGTGCATTGGCAAAGAGCTTGAAAGAGAAGGAACTCTCTGAGGG
AGCCTACACCCTGACACTTGAGACTATATCAGGCTCTCTCCAGGCTGCTCTCCAGTCACTTTTC
AAATACACTAGAGCAAGAACAGGGGATCGAACGCTGATAGATGCCCTTGAGCCATTTGTAAAAG
AATTCGCAAAATCAAAAGATTTAAAACTGGCAAACAAAGCCGCTCACGACGGAGCAGAAGCGAC
CAGAAAACTTGAAGCGAAATTTGGTAGAGCTTCGTACGTGGCTGAGGAAGAATTCAAGCAATTT
GAGTCTGAGGGTGGACTCCCTGACCCAGGAGCAATTGGGCTGGCCGCTTTAATTTCCGGTATCA
CTGACGCCTATTTCAAGTCGGAAACGAAGCTCTAG
-Exemplary Komagataella phaffii GS115 (Pischia pastoris)
Dihydroxyacetone Kinase (DAKP) Amino Acid Sequence
SEQ ID NO: 210
MSSKHWDYKKDLVLSHLAGLCQSNPHVRLIESERVVISAENQEDKITLISGGGSGHEPLHAGFV
TKDGLLDAAVAGFIFASPSTKQIFSAIKAKPSKKGTLIIVKNYTGDILHFGLAAEKAKAEGLNA
ELLIVQDDVSVGKAKNGLVGRRGLAGTSLVHKILGAKAYLQKDNLELHQLVTFGEKVVANLVTI
GASLDHVTIPARANKQEEDDSDDEHGYEVLKHDEFEIGMGIHNEPGIKKSSPIPTVDELVAELL
EYLLSTTDKDRNYVQFDKNDEVVLLINNLGGTSVLELYAIQNIVVDQLASKYSIKPVRIFTGTF
TTSLDGPGFSITLLNATKTGDKDILKFLDHKTSAPGWNSNISDWSGRVDNFIVAAPEIDEGDSS
SKVSVDAKLYADLLESGVKKVISKEPKITLYDTVAGDGDCGETLANGSNAILKALAEGKLDLKD
GVKSLVQITDIVETAMGGTSGGLYSIFISALAKSLKEKELSEGAYTLTLETISGSLQAALQSLF
KYTRARTGDRTLIDALEPFVKEFAKSKDLKLANKAAHDGAEATRKLEAKFGRASYVAEEEFKQF
ESEGGLPDPGAIGLAALISGITDAYFKSETKL
-Exemplary Escherichia coli Dihydroxyacetone Kinase (DAKE) Nucleic
Acid Coding Sequence
SEQ ID NO: 211
ATGAAAAAATTGATCAATGATGTGCAAGACGTACTGGACGAACAACTGGCAGGACTGGCGAAAG
CGCATCCATCGCTGACACTGCATCAGGATCCGGTGTATGTCACCCGAGCTGATGCCCCTGTTGC
AGGAAAAGTCGCCCTGCTGTCGGGTGGCGGCAGCGGACACGAGCCGATGCACTGTGGGTATATC
GGTCAGGGGATGCTTTCGGGGGCCTGTCCGGGCGAAATTTTCACCTCACCGACGCCCGATAAAA
TCTTTGAATGCGCCATGCAAGTTGATGGCGGCGAAGGTGTACTGTTGATTATCAAAAATTACAC
CGGCGATATTCTTAACTTTGAAACAGCGACCGAGTTACTGCACGATAGCGGCGTAAAAGTGACC
ACTGTGGTCATTGATGACGACGTTGCGGTAAAAGACAGTCTTTATACTGCCGGGCGACGCGGCG
TTGCCAACACCGTATTAATTGAAAAACTCGTAGGCGCAGCGGCGGAGCGTGGCGACTCACTGGA
CGCCTGTGCGGAACTGGGGCGTAAGCTGAATAATCAAGGCCACTCAATAGGTATCGCTCTCGGT
GCCTGTACCGTTCCTGCCGCGGGCAAACCTTCTTTTACCCTGGCGGATAATGAGATGGAGTTTG
GCGTCGGCATTCATGGTGAGCCGGGTATTGACCGCCGCCCCTTCTCTTCCCTTGATCAAACCGT
CGATGAAATGTTCGACACCCTGCTGGTAAATGGCTCATACCATCGCACTTTGCGTTTCTGGGAT
TATCAACAAGGCAGTTGGCAGGAAGAACAACAAACCAAACAACCGCTCCAGTCTGGCGATCGGG
TGATTGCGCTGGTTAACAATCTTGGCGCAACTCCGCTTTCTGAGCTGTACGGCATCTATAACCG
CCTGACCACACGTTGCCAGCAAGCGGGATTGACTATCGAACGTAATTTAATTGGCGCGTACTGC
ACCTCACTGGATATGACCGGTTTCTCAATCACCTTACTGAAAGTTGATGACGAAACGCTGGCAC
TCTGGGACGCCCCGGTCCACACCCCGGCCCTTAACTGGGGTAAATAA
-Exemplary Escherichia coli Dihydroxyacetone Kinase (DAKE) Amino
Acid Sequence
SEQ ID NO: 212
MKKLINDVQDVLDEQLAGLAKAHPSLTLHQDPVYVTRADAPVAGKVALLSGGGSGHEPMHCGYI
GQGMLSGACPGEIFTSPTPDKIFECAMQVDGGEGVLLIIKNYTGDILNFETATELLHDSGVKVT
TVVIDDDVAVKDSLYTAGRRGVANTVLIEKLVGAAAERGDSLDACAELGRKLNNQGHSIGIALG
ACTVPAAGKPSFTLADNEMEFGVGIHGEPGIDRRPFSSLDQTVDEMFDTLLVNGSYHRTLRFWD
YQQGSWQEEQQTKQPLQSGDRVIALVNNLGATPLSELYGIYNRLTTRCQQAGLTIERNLIGAYC
TSLDMTGFSITLLKVDDETLALWDAPVHTPALNWGK
-Exemplary Citrobacter freundii Dihydroxyacetone Kinase (DHAKC)
Nucleic Acid Coding Sequence
SEQ ID NO: 213
ATGTCTCAATTCTTCTTCAATCAAAGAACACACCTTGTATCTGACGTTATTGACGGGACCATTA
TAGCATCACCTTGGAATAACTTGGCCAGGCTAGAGAGCGATCCAGCGATTAGGATAGTCGTGAG
ACGTGATTTGAATAAGAACAACGTTGCTGTTATCAGTGGAGGAGGGTCTGGACATGAGCCAGCT
CATGTAGGTTTCATAGGGAAAGGAATGCTAACTGCCGCTGTTTGCGGAGACGTGTTCGCTTCAC
CAAGTGTCGACGCCGTTCTAACGGCGATTCAGGCAGTCACAGGTGAGGCAGGATGTCTCCTAAT
TGTCAAGAATTACACCGGAGACAGACTTAATTTCGGTTTGGCTGCAGAGAAGGCTCGTAGACTG
GGCTATAACGTCGAGATGCTAATAGTGGGCGACGATATTTCATTACCAGATAACAAGCACCCTA
GAGGGATCGCGGGTACCATATTAGTTCACAAGATCGCAGGGTACTTCGCAGAAAGAGGATATAA
TCTAGCGACTGTTTTGCGAGAGGCACAGTACGCGGCTAACAATACTTTTAGTCTTGGGGTAGCG
TTGTCCTCATGTCATCTCCCTCAAGAGGCGGACGCCGCGCCTAGGCATCACCCAGGACACGCAG
AACTTGGCATGGGCATACACGGCGAGCCGGGAGCGTCTGTTATCGATACGCAAAATTCAGCTCA
GGTTGTTAATCTGATGGTTGACAAACTCATGGCTGCGTTACCGGAAACAGGGCGACTCGCAGTC
ATGATAAATAACCTGGGTGGTGTGAGCGTAGCTGAAATGGCGATCATCACACGGGAGCTGGCTT
CTTCACCTCTTCACCCAAGGATCGACTGGCTCATAGGGCCAGCAAGCTTGGTTACCGCATTAGA
TATGAAATCTTTCAGCTTAACAGCAATCGTACTAGAGGAAAGCATTGAGAAAGCACTTCTCACA
GAGGTGGAGACATCAAATTGGCCAACGCCGGTGCCCCCTAGAGAAATTTCGTGCGTGCCTTCAA
GTCAGCGGAGTGCTCGTGTTGAATTTCAGCCCTCAGCGAACGCTATGGTTGCAGGGATTGTAGA
ACTGGTGACTACAACTTTATCGGACCTCGAAACACACTTAAATGCCTTGGACGCCAAAGTTGGA
GACGGCGATACGGGATCAACCTTCGCTGCAGGGGCGCGGGAAATAGCAAGTCTCTTGCACCGAC
AACAGCTCCCGTTAGATAATTTGGCTACACTCTTCGCATTGATCGGAGAACGTCTCACAGTAGT
AATGGGTGGTTCCAGTGGGGTTTTAATGTCGATCTTCTTCACTGCTGCAGGTCAAAAGCTCGAA
CAAGGAGCATCGGTGGCTGAAAGTCTGAACACCGGATTAGCACAGATGAAATTCTACGGTGGAG
CCGATGAGGGTGATCGTACTATGATCGATGCGCTGCAGCCCGCATTAACTTCGCTCTTAACGCA
GCCACAAAATCTTCAGGCAGCTTTCGACGCTGCCCAAGCAGGGGCGGAACGTACCTGTTTGAGC
TCTAAGGCTAATGCGGGACGTGCGTCATATCTTTCATCGGAGAGTCTCCTTGGTAACATGGACC
CCGGAGCACACGCAGTAGCTATGGTGTTTAAGGCCTTAGCGGAGTCTGAGCTCGGATAG
-Exemplary Citrobacter freundii Dihydroxyacetone Kinase (DHAKC)
Amino Acid Sequence
SEQ ID NO: 214
MSQFFFNQRTHLVSDVIDGTIIASPWNNLARLESDPAIRIVVRRDLNKNNVAVISGGGSGHEPA
HVGFIGKGMLTAAVCGDVFASPSVDAVLTAIQAVTGEAGCLLIVKNYTGDRLNFGLAAEKARRL
GYNVEMLIVGDDISLPDNKHPRGIAGTILVHKIAGYFAERGYNLATVLREAQYAANNTFSLGVA
LSSCHLPQEADAAPRHHPGHAELGMGIHGEPGASVIDTQNSAQVVNLMVDKLMAALPETGRLAV
MINNLGGVSVAEMAIITRELASSPLHPRIDWLIGPASLVTALDMKSFSLTAIVLEESIEKALLT
EVETSNWPTPVPPREISCVPSSQRSARVEFQPSANAMVAGIVELVTTTLSDLETHLNALDAKVG
DGDTGSTFAAGAREIASLLHRQQLPLDNLATLFALIGERLTVVMGGSSGVLMSIFFTAAGQKLE
QGASVAESLNTGLAQMKFYGGADEGDRTMIDALQPALTSLLTQPQNLQAAFDAAQAGAERTCLS
SKANAGRASYLSSESLLGNMDPGAHAVAMVFKALAESELG

### E) Formate Pathway

In some embodiments, compositions and methods described herein comprise introduction of one or more genes coding for HCHO metabolism into CO₂ through a formate intermediate, which is then taken up by various endogenous pathways, for example the Calvin Benson cycle. In some embodiments, these enzymes metabolize the substrate formate to produce CO₂, a component that can be incorporated into the Calvin-Benson cycle, a photosynthetic carbon fixation pathway, or other endogenous plant pathways. In some embodiments, genes are introduced that comprise coding sequences for formaldehyde dehydrogenase (FALDH) and/or formate dehydrogenase (FDH). In certain embodiments, Serine hydroxymethyltransferase 1, mitochondrial (SHM1) and/or(S)-2-hydroxy-acid oxidase (GLO1 and/or GLO2) may also impact the metabolic flux of HCHO metabolism as described herein, for example, through the production of L-Serine and/or oxocarboxylate. In some embodiments, genes are introduced that comprise coding sequences for SHM1, GLO1, and/or GLO2.

### Formaldehyde Dehydrogenase (FALDH)

In certain embodiments, a composition described herein comprises at least one transgenic FALDH enzyme. In some embodiments, FALDH enzymes utilize the substrate formaldehyde, and create the product formate.

In some embodiments, a FALDH gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 216, 218, or 220 (or a portion thereof). In some embodiments, a FALDH gene and/or transgene comprises a nucleotide sequence that is at least 90%, 91%, 92%, 93%, 94%, 95%, 96%, 97%, 98%, 99%, or 100% identical to any one of SEQ ID NOs: 215, 217, or 219 (or a portion thereof).

-Exemplary Methylobacterium sp. XJLW Formaldehyde dehydrogenase,
glutathione-independent (FALDH9) Nucleic Acid Coding Sequence
SEQ ID NO: 215
ATGGCCGCTAACGGAAACAGGGTCGTTACTTTTCAGGGTCCTATGAAAATGGAACTAAAGACTT
TCGATTTTCCTAAATTGGTCACACCAACTGGGAAGAAAGCAAATCACGGGGCTATTTTGAAAAT
AGTGACCACCAACATTTGCGGATCTGACCAGCACATTTATCACGGTCGGTTCGCCGCACCAAAA
GGGATGGTTATGGGACACGAAATGACGGGCGAAGTTATTGAGGTCGGGTCTGATGTTGAGTTTA
TTAGAGTGGGTGACTTATGCAGTGTACCGTTTAATGTATCCTGCGGGCGGTGCAGGAACTGCAA
AGAAAGGCACACTGATGTATGTATGAATGTTAATGATGAGGTAGACTGCGGCGCGTATGGATTC
AATCTCGGTGGATGGCAAGGTGGGCAGTCCGACTACCTCATGGTACCTTACGCGGATTGGAACC
TTCTCTCGTTCCCGGACAAGGACCAAGCAATGGAGAAGATTAGAGATCTGACATTGTTGTCTGA
CATACTTCCTACCGGTTTCCACGGTCTTATGGCCGCAGGCGCTAAAGCTGGATCGACTGTGTAT
ATCGCTGGAGCTGGGCCTGTCGGCAGGTGCGCAGCTGCTGGGGCAAGATTGATTGGGGCGTCCT
GTATCATCGTTGCCGACACGAACCGAGCTAGGTTGGACTTGGTTAAGAACAATGGTTGCGAGGT
GGTCGACCTCACGAAGGGTACACCTGTACCTGACCAAATAGAGGCGATCCTCGGTAAGAGAGAA
GTTGATTGTGGTGTGGATTGTGTTGGCCTCGAAGCACATGGTAATGGACCTGAGGCTAACAAGG
AGCATTCAGAAGCTGTTATAAACACGCTTTTCCAAGTCGTGAGAGCAGGTGGGGCGATGGGAGT
TCCTGGAATCTATACAGCTGCGGACCCGAAGGCATCTTCAGAATTGACAAAGAAAGGACAGTTG
CCTATAGACTTTGGAAAGGCATGGATTAAGTCTCCAAAGTTGACAGCAGGTCAGGCCCCTATAA
TGCACTATAATCGGGATCTGATGATGGCTATATTGTGGGACAGGATGCCATACCTGGGAGCAAT
GCTCAACACAGAAGTAATTACTTTAGAGCAAGCACCAGCCGCTTATAAGACGTTCTCAGACGGT
AGTCCTAAGAAGTTTGTTATCGACCCCCACGGGTCCGTTAAGAAGGCATCGTAG
-Exemplary Methylobacterium sp. XJLW Formaldehyde dehydrogenase,
glutathione-independent (FALDH9) Amino Acid Sequence
SEQ ID NO: 216
MAANGNRVVTFQGPMKMELKTFDFPKLVTPTGKKANHGAILKIVTTNICGSDQHIYHGRFAAPK
GMVMGHEMTGEVIEVGSDVEFIRVGDLCSVPFNVSCGRCRNCKERHTDVCMNVNDEVDCGAYGF
NLGGWQGGQSDYLMVPYADWNLLSFPDKDQAMEKIRDLTLLSDILPTGFHGLMAAGAKAGSTVY
IAGAGPVGRCAAAGARLIGASCIIVADTNRARLDLVKNNGCEVVDLTKGTPVPDQIEAILGKRE
VDCGVDCVGLEAHGNGPEANKEHSEAVINTLFQVVRAGGAMGVPGIYTAADPKASSELTKKGQL
PIDFGKAWIKSPKLTAGQAPIMHYNRDLMMAILWDRMPYLGAMLNTEVITLEQAPAAYKTFSDG
SPKKFVIDPHGSVKKAS
-Exemplary Pseudomonas sp. 101 Formaldehyde dehydrogenase
(FALDHP) Nucleic Acid Coding Sequence
SEQ ID NO: 217
ATGAGTGGTAACCGAGGCGTAGTGTACTTGGGTTCAGGAAAGGTAGAAGTCCAGAAGATTGATT
ATCCAAAGATGCAGGACCCTAGGGGTAAGAAAATCGAGCACGGCGTAATACTGAAAGTAGTGTC
CACCAACATTTGCGGTTCTGACCAGCATATGGTAAGAGGGCGAACTACAGCGCAGGTAGGTTTG
GTTCTCGGGCACGAAATAACTGGTGAGGTTATAGAGAAAGGTAGAGATGTTGAAAATCTGCAGA
TAGGAGATCTTGTCTCGGTGCCATTCAACGTGGCTTGTGGGCGGTGCAGGAGTTGCAAGGAAAT
GCACACAGGGGTCTGCCTTACTGTTAATCCAGCGCGAGCTGGCGGGGCGTATGGTTACGTTGAC
ATGGGTGACTGGACTGGTGGACAAGCAGAATACCTTCTCGTCCCATACGCGGACTTCAACTTAC
TCAAATTGCCGGACCGTGACAAGGCTATGGAAAAGATAAGGGACCTCACCTGCCTATCAGACAT
ACTGCCGACAGGATATCATGGTGCAGTCACTGCTGGAGTAGGTCCAGGCTCGACAGTTTACGTT
GCGGGTGCAGGACCGGTGGGTCTTGCTGCTGCAGCGTCGGCGAGACTGTTGGGAGCAGCAGTTG
TTATAGTTGGCGATTTGAACCCGGCCAGACTCGCGCATGCTAAAGCGCAAGGTTTTGAAATAGC
GGACCTCTCATTGGACACCCCGTTACATGAGCAGATTGCAGCACTCCTGGGTGAACCAGAAGTT
GATTGCGCGGTCGATGCTGTTGGATTCGAAGCTAGAGGACACGGTCACGAAGGAGCAAAACATG
AGGCACCCGCTACAGTACTAAATAGTCTAATGCAAGTTACCAGAGTTGCGGGGAAGATAGGTAT
CCCAGGATTATACGTGACTGAAGATCCAGGTGCAGTGGACGCAGCAGCCAAGATCGGTTCTCTA
AGTATCCGATTTGGTTTGGGATGGGCCAAATCGCATTCTTTTCACACGGGGCAAACCCCTGTAA
TGAAGTATAATCGGGCCTTGATGCAAGCTATTATGTGGGATCGTATAAACATCGCTGAGGTCGT
AGGAGTCCAAGTAATCAGTCTTGACGACGCTCCACGAGGGTATGGAGAGTTCGACGCTGGGGTG
CCTAAGAAATTTGTTATCGACCCTCACAAAACATTTTCGGCAGCTTAG
-Exemplary Pseudomonas sp. 101 Formaldehyde dehydrogenase
(FALDHP) Amino Acid Sequence
SEQ ID NO: 218
MSGNRGVVYLGSGKVEVQKIDYPKMQDPRGKKIEHGVILKVVSTNICGSDQHMVRGRITAQVGL
VLGHEITGEVIEKGRDVENLQIGDLVSVPFNVACGRCRSCKEMHTGVCLTVNPARAGGAYGYVD
MGDWTGGQAEYVLVPYADFNLLKLPDRDKAMEKIRDLTCLSDILPTGYHGAVTAGVGPGSTVYV
AGAGPVGLAAAASARLLGAAVVIVGDLNPARLAHAKAQGFEIADLSLDTPLHEQIAALLGEPEV
DCAVDAVGFEARGHGHEGAKHEAPATVLNSLMQVTRVAGKIGIPGLYVTEDPGAVDAAAKIGSL
SIRFGLGWAKSHSFHTGQTPVMKYNRALMQAIMWDRINIAEVVGVQVISLDDAPRGYGEFDAGV
PKKFVIDPHKTFSAA
-Exemplary Epipremnum Aureum Formaldehyde dehydrogenase
(FALDHEa) Nucleic Acid Coding Sequence
SEQ ID NO: 219
ATGGCTACTAAGCGCAAGTCATAACATGTAAAGCCGCTGTTGCGTGGGAAGCCAATAAACCCCT
AGCGATCGAGGATGTCCTCGTTGCACCACCTCAAGCCGGAGAAGTCCGCATTAAAATCCTTTTT
ACCGCTTTGTGTCATACCGATGCGTATACGTGGAGCGGGAAGGATCCTGAAGGGCTGTTTCCAT
GTATTTTGGGACATGAAGCCGCAGGGATAGTGGAATCGGTCGGAGAGGGAGTCACCGAAGTTCA
ACCAGGTGACCATGTAATCCCATGCTATCAGGCTGAATGTAGGGAGTGCAAATTTTGCAAATCA
GGTAAGACTAATTTATGTGGTAAAGTTCGTGCAGCTACGGGCGTTGGAATTATGATGAATGATA
GAAAGAGCAGATTTTCTATAAATGGTAAACCAATTTATCACTTTATGGGGACGAGTACGTTTTC
ACAATATACCGTAGTTCATGATGTTTCTGTTGCCAAAATTGATCCCAAAGCACCACTCGAGAAG
GTTTGTCTACTTGGGTGTGGTGTTGCAACAGGGTTGGGAGCAGTATGGAACACAGCCAAAGTCG
AGGCTGGCTCCATCGTAGCCATATTTGGTCTTGGAACTGTAGGTTTGGCCGTAGCTGAAGGAGC
AAAAACCGCAGGAGCGAGCCGAATAATTGGAATAGATATTGACAGCAAGAAATTCGACGTAGCC
AAAAATTTTGGAGTTACAGAGTTTGTTAACCCAAAAGATTATGAGAAACCGATCCAGCAAGTTT
TGGTAGACCTCACTGACGGAGGCGTGGACTATTCCTTTGAATGCATAGGAAACGTATCAGTTAT
GCGAGCCGCATTAGAATGCTGTCACAAGGGGTGGGGGACGAGCGTTATCGTCGGGGTTGCTGCA
TCAGGGCAAGAGATTTCCACTAGACCATTTCAGTTGGTCACCGGCCGAGTGTGGAAAGGTACAG
CATTTGGAGGGTTTAAGTCCCGCAGCCAGGTCCCCTGGCTGGTAGATAAGTATATGAAGAAAGA
GATCAAAGTGGATGAGTACATTACACATAATCTGACATTGGGAGAAATAAACAAAGGTTTCGAC
TTTATGCATGAAGGGAGCTGTCTCAGATGTGTGTTAGATACTCAAGTATAA
-Exemplary Epipremnum Aureum Formaldehyde dehydrogenase
(FALDHEa)Amino Acid Sequence
SEQ ID NO: 220
MATEAQVITCKAAVAWEANKPLAIEDVLVAPPQAGEVRIKILFTALCHTDAYTWSGKDPEGLFP
CILGHEAAGIVESVGEGVTEVQPGDHVIPCYQAECRECKFCKSGKTNLCGKVRAATGVGIMMND
RKSRFSINGKPIYHFMGTSTFSQYTVVHDVSVAKIDPKAPLEKVCLLGCGVATGLGAVWNTAKV
EAGSIVAIFGLGTVGLAVAEGAKTAGASRIIGIDIDSKKFDVAKNFGVTEFVNPKDYEKPIQQV
LVDLTDGGVDYSFECIGNVSVMRAALECCHKGWGTSVIVGVAASGQEISTRPFQLVTGRVWKGT
AFGGFKSRSQVPWLVDKYMKKEIKVDEYITHNLTLGEINKGFDFMHEGSCLRCVLDTQV

### Glutathione-Dependent Formaldehyde Dehydrogenase (GD-FALDH)

In certain embodiments, a composition described herein comprises at least one transgenic GD-FALDH enzyme. In some embodiments, GD-FALDH enzymes utilize the substrate formaldehyde, and create the product formate.

In some embodiments, a GD-FALDH gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 222 or 224 (or a portion thereof). In some embodiments, a GD-FALDH gene and/or transgene comprises a nucleotide sequence that is at least 90%, 91%, 92%, 93%, 94%, 95%, 96%, 97%, 98%, 99%, or 100% identical to any one of SEQ ID NOs: 221 or 223 (or a portion thereof).

-Exemplary Methylobacterium sp. XJLW Formaldehyde dehydrogenase
(GD-FALDH10) Nucleic Acid Coding Sequence
SEQ ID NO: 221
ATGAAGGCACTGTGCTGGCACGGCCGCAACGATATCCGCTGCGACACGGTCCCGGACCCGGTCA
TCGAGGATTCCCGCGACGTGATCATCAAGGTCACGAGCTGCGCGATCTGCGGCTCGGACCTACA
TCTGATGGACGGCCAGATGCCGACCATGAAGAGCGGCGACGTCCTCGGCCACGAATTCATGGGC
GAGATCGTGGAGGTCGGGACCGGCTTCACCAAGTTCAAAAAGGGCGATCGGATCGTCGTGCCCT
TCAACATCAACTGCGGCGCATGCCGCCAGTGCAAGCTCGGCAATTACTCGGTCTGCGAGCGCTC
AAACCGCAACGCCGAGATGGCGGCCGCGCAGTTCGGCTACACGACGGCCGGCCTGTTCGGATAC
TCGCACCTGACCGGCGGCTATGCCGGTGGCCAGGCCGAGTATGTCCGTGTGCCGATGGCCGACG
TCGCGCCAATGAAGGTGCCGGAAGGCATGGACGACGAATCCGTCCTGTTCCTCACCGACATCCT
GCCCACCGGCTGGCAGGGCGCGGAGCATTGCGAGATCCAGGGCGGCGAGACGATTGCGGTCTGG
GGCGCCGGCCCGGTCGGCATCTTCGCGATCCAATCGGCGAAGATCATGGGGGCCGAGCGGATCA
TCGCCATCGAGACCGTGCCCGAGCGCATCGCCCTCGCCCGGAAGGCCGGCGCCACCGACATCAT
CGACTTCATGAACGAGGACGTGTTCGAGCGAATCAAGGAGATCACCAAGGGCCAGGGTGCCGAC
GGCGTGATCGACTGCGTCGGCATGGAGGCGAGTGCCGGCCATGGCGGCCTCACTGGCGTGCTCT
CCGCCGTCCAGGAGAAGCTGACCGCCACCGAGCGGCCCTACGCGCTGGCCGAAGCCATCAAGGC
GGTCCGGCCCTGTGGGATCGTCTCGGTGCCCGGCGTCTATGGCGGACCGATCCCGGTCAACATG
GGCTCGATCGTCCAGAAGGGCCTGACCCTCAAGAGCGGCCAGACCCATGTGAAGCGCTATCTCG
AGCCGCTGACCAAGCTGATCCAAGAGGGCAAGATCGACATGACCTCCCTGATCACCCACCGCTC
GCACGACCTCGCGGATGGGCCGGACCTCTACAAGGCCTTCCGCGACAAGAAGGACGGCTGCGTG
AAGGTGGTGTTTCACCTGAACTGA
-Exemplary Methylobacterium sp. XJLW Formaldehyde dehydrogenase
(GD-FALDH10) Amino Acid Sequence
SEQ ID NO: 222
MKALCWHGRNDIRCDTVPDPVIEDSRDVIIKVTSCAICGSDLHLMDGQMPTMKSGDVLGHEFMG
EIVEVGTGFTKFKKGDRIVVPFNINCGACRQCKLGNYSVCERSNRNAEMAAAQFGYTTAGLFGY
SHLTGGYAGGQAEYVRVPMADVAPMKVPEGMDDESVLFLTDILPTGWQGAEHCEIQGGETIAVW
GAGPVGIFAIQSAKIMGAERIIAIETVPERIALARKAGATDIIDEMNEDVFERIKEITKGQGAD
GVIDCVGMEASAGHGGLTGVLSAVQEKLTATERPYALAEAIKAVRPCGIVSVPGVYGGPIPVNM
GSIVQKGLTLKSGQTHVKRYLEPLTKLIQEGKIDMTSLITHRSHDLADGPDLYKAFRDKKDGCV
KVVFHLN
-Exemplary Methylobacterium sp. XJLW Formaldehyde dehydrogenase
(GD-FALDH11) Nucleic Acid Coding Sequence
SEQ ID NO: 223
ATGAAAGCTCTTACTTGGCAAAGTCGAGGGAAAATTACTTGTGAAACAGTCCCTGACCCTAAAA
TCGAGCACGGGCGAGATGTGATCATTAAAGTAACGGCTTGTGCTATCTGTGGTAGTGATCTACA
CCTCATGGGTGGGTTTATGCCGACTATGAAATGCGGAGATATCCTTGGACATGAGACAATGGGA
GAGGTCATAGAGGTTGGTAAGGACAACCATAAGCTTAAAGTTGGTGACCGTATAGTCGTTCCGT
TCACAATCTGTTGCGGAGAATGCCGGCAATGCAAATGGGGTAACTGGAGCTGCTGCGAACGGAC
TAACCCTAACGGCAAACTGCAAGCTGAGACATACGGTTATCCTCTCGCCGGGTTGTTCGGATTT
TCACACATCACAGGCGGTTTCGCTGGCGGGCAAGCAGAGTATTTAAGAGTGCCTTATGCAGATG
TGGGGCCCATTGTCGTACCAGAAGGACTCACGGACGAGCAAGTCCTGTTTCTTTCAGACATATT
TCCTACTGCTTACCAGGCCGCAGAGCATTGCGACATCGGGCCAGAGGATACAGTCGCCATTTGG
GGTTGCGGTCCAGTAGGGGTGCTCGCTGTGAAGTGTTGCTATCTACTTGGAGCAAAGAGAGTTA
TTGCAATTGATTCAGTGCCGGAGAGGCTTGCGCTCGCACGAGAAGCTGGTGCTGAGACAATCGA
TCTTTCATCTCAAAATGTCCAGGACACCCTCATGGAGATGACACACGGACTTGGTCCTGACTCC
GTCATCGAGGCAGTCGGGATGGAAAGCCACGGTGCTGACACAACACTTCAAAAGGTATCTTCTG
CTATCATGGAGCACACTGTTTCGTTAGAAAGGCCATTTGCGCTCAACCAAGCTATCCTCGCCTG
CAGGCCTGGCGGTAATGTCTCTATGCCAGGGGTTTTCGCGGGTCCTGTGGGACCAGTCGCACTA
GGAGTGCTGATGAATAAGGGACTCACTCTTAAAACCGGCCAGACACATATGGTGCGGTATATGA
AGCCTCTATTAGAGAGGATTCAGAAGGGTGAGATAGACCCATCATTTATCGTGTCCCATCGATC
GACAAACTTGGAAGAAGGTCCCGCACTTTACGAGGCCTTTCGAGATAAAACCGACAATTGCACC
AAAGTGGTGTTTAAACCCCATTAG
-Exemplary Methylobacterium sp. XJLW Formaldehyde dehydrogenase
(GD-FALDH11) Amino Acid Sequence
SEQ ID NO: 224
MKALTWQSRGKITCETVPDPKIEHGRDVIIKVTACAICGSDLHLMGGFMPTMKCGDILGHETMG
EVIEVGKDNHKLKVGDRIVVPFTICCGECRQCKWGNWSCCERINPNGKLQAETYGYPLAGLFGF
SHITGGFAGGQAEYLRVPYADVGPIVVPEGLTDEQVLFLSDIFPTAYQAAEHCDIGPEDTVAIW
GCGPVGVLAVKCCYLLGAKRVIAIDSVPERLALAREAGAETIDLSSQNVQDTLMEMTHGLGPDS
VIEAVGMESHGADTTLQKVSSAIMEHTVSLERPFALNQAILACRPGGNVSMPGVFAGPVGPVAL
GVLMNKGLTLKTGQTHMVRYMKPLLERIQKGEIDPSFIVSHRSTNLEEGPALYEAFRDKIDNCT
KVVFKPHG

### Formate Dehydrogenase (FDH)

In certain embodiments, a composition described herein comprises at least one transgenic FDH enzyme. In some embodiments, FDH enzymes utilize the substrate formate, and create the product CO2.

In some embodiments, a FDH gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 226, 227, 228, 229, 231, 233, 234, 236, 238, or 240 (or a portion thereof). In some embodiments, a FDH gene and/or transgene comprises a nucleotide sequence that is at least 90%, 91%, 92%, 93%, 94%, 95%, 96%, 97%, 98%, 99%, or 100% identical to any one of SEQ ID NOs: 225, 230, 232, 235, 237, or 239 (or a portion thereof).

-Exemplary Methylobacterium sp. XJLW Formate Dehydrogenase
(FDH3) Nucleic Acid Coding Sequence
SEQ ID NO: 225
ATGAGCGTGACTCTCTATATTCCTCGGGATGCAGTGGCCTTGGGTCTTGGTGCGAACAAGGTAG
CTAGAGCGTTGTTCGCAGGAGCTGAACGTCGGGGTCTAGATGTAACCATCGTGCGAACAGGAAG
TCGAGGACTTTTCTGGTTAGAGCCAATGGTTGAGGTGGGAACACCAGAGGGAAGAGTAGCGTAT
GGACCCGTAAAGCTGGCAGACATAGACGCTCTTCTTGATGCTGGGCTCGCAACCGGCGGAGATC
ATCCACTACGATTAGGTGACCCTGAAAAGATCCCTTACTTAGCTCGGCAACAACGGTTAACCTT
TCACAGGTGCGGTGTTATTGATCCTGTTAGTGTGGACGATTATCGTGCCCATGGTGGTTATCGA
GGCCTAGAAGCAGCTCTCAAACTCGATGCTGAAGGTATCGTAGCGGCAGTAAGGGACTCCGGAC
TCCGTGGACGGGGTGGTGCAGGCTTCCCAGCCGGAATTAAATGGAATACGGTTATGCTAGCTAA
AGCTGACCAGAAGTATGTAGTTTGTAACGCAGACGAGGGTGACTCAGGTACTTTTGCAGACAGA
ATGATGATGGAAGGAGATCCCTTTAATCTAATCGAAGGCATGACCATCGCAGCCGTCGCTACTG
GAGCAACCAGAGGATACATATACCTTAGGTCGGAATATCCACAGGCCTTTGCAACACTGAAGGA
AGCTATCGCGAACGGAGTGACTGCAGGAGTCCTCGGTGAGAATATATTAGGATCAGGGAAAACT
TTTCACTTAGAGGTGAGATTAGGAGCCGGTGCGTACATTTGCGGTGAAGAGACGTCACTACTTG
AGTCTCTAGAGGGTAAGAGAGGAATCGTCCGTGCTAAACCACCTATTCCAGCTCTCAAAGGATT
CTTAGGTAAACCGACGTTGGTAAATAACGTAATGACCTTTACAGCAGTTCCTTGGATATTGGAG
AATGGAGCAAAGGCGTATGCGGATTACGGCATGGGACGTAGTTTGGGCACCTTGCCGATTCAAC
TCGCAGGTAACATCAAACACGGTGGTTTGATCGAAATGGCCTTTGGAATCACTTTGCGTCAGGT
CATCGAGGACTTTGGAGGAGGTACACGGTCTGGTCGTCCAGTGCGTGCCGTGCAAGTAGGTGGT
CCACTGGGCGCCTATTTTCCAGATCACCTCTTAGACACCCCGCTCGACTACGAGGCAATGGCAG
CAAAGAAAGGCCTGGTTGGACACGGTGGCATCGTTGTCTTTGATGACACGGTTGACATGGCAGC
GCAAGCGCGATTTGCCTTTGAGTTCTGCGCTACCGAATCTTGTGGAAAATGCACACCGTGCAGA
ATCGGTGCGACACGAGGGGTCGAAACAATGGATAAGGTGATAGCAGGAATCCGACCAGACGCGA
ACCTCAAACTCGTTGAGGATTTGTGCGAGGTAATGACAGATGGTTCTCTGTGTGCTATGGGTGG
GCTCACGCCTATGCCAGTTATGAGCGCAATCACCCACTTTCCGGAAGATTTCCGTCGAGCCGGA
GACTTGCCGGCTGCAGCCGAGTAA
-Exemplary Methylobacterium sp. XJLW Formate Dehydrogenase
(FDH3) Amino Acid Sequence
SEQ ID NO: 226
MSVTLYIPRDAVALGLGANKVARALFAGAERRGLDVTIVRTGSRGLFWLEPMVEVGTPEGRVAY
GPVKLADIDALLDAGLATGGDHPLRLGDPEKIPYLARQQRLTFHRCGVIDPVSVDDYRAHGGYR
GLEAALKLDAEGIVAAVRDSGLRGRGGAGFPAGIKWNTVMLAKADQKYVVCNADEGDSGTFADR
MMMEGDPFNLIEGMTIAAVATGATRGYIYLRSEYPQAFATLKEAIANGVTAGVLGENILGSGKT
FHLEVRLGAGAYICGEETSLLESLEGKRGIVRAKPPIPALKGFLGKPTLVNNVMTFTAVPWILE
NGAKAYADYGMGRSLGTLPIQLAGNIKHGGLIEMAFGITLRQVIEDFGGGTRSGRPVRAVQVGG
PLGAYFPDHLLDTPLDYEAMAAKKGLVGHGGIVVFDDTVDMAAQARFAFEFCATESCGKCTPCR
IGATRGVETMDKVIAGIRPDANLKLVEDLCEVMTDGSLCAMGGLTPMPVMSAITHFPEDFRRAG
DLPAAAE
-Exemplary Methylobacterium sp. XJLW Formate Dehydrogenase
Subunit Alpha (FDH4) Amino Acid Sequence
SEQ ID NO: 227
MSNAPEQHGDKTEKSEIRADGLQDAGGPAQGPKPEAGGSYSEGAKAGGQAAPEPSGLHDLKGRP
TAPPTIAFELDGQQVEAAPGETIWAVAKRLGTHIPHLCHKPEPGYRPDGNCRACMVEIEGERVL
AASCKRTPAVGMKVKTATERATKARAMVLELLVADQPERETSHDPTSHFWVQADFLDVSESRFP
AAERWTGDFSHPAMSVNLDACIQCNLCVRACREVQVNDVIGMAYRSAGAKVVFDFDDPMGGSTC
VACGECVQACPTGALMPSAYLDAEHKTRTVYPDREVTSLCPYCGVGCQVSYKVKDEKIVYAEGV
NGPANHNRLCVKGRFGFDYVHHPHRLTAPLIRLDNIPKDANDQVDPANPWTHFREATWEEALDR
AAGGLKTVRDTHGRKALAGFGSAKGSNEEAYLFQKLVRLGFGSNNVDHCTRLCHASSVAALMEG
LNSGAVSAPFSAALDAEVIIVIGANPTVNHPVAATFLKNAVKQRGAKLIVMDPRRQVLSRHAYK
HLAFKPGSDVAMLNAMLNVIIEERLYDEQYIAGYTENFEALKEKIVEFTPEKMASVCGIDAETL
REVARLYARAKSSIIFWGMGISQHVHGTDNSRCLIALALVTGQIGRPGTGLHPLRGQNNVQGAS
DAGLIPMVYPDYQSVEKAAVREMFEEFWGQKLDPQRGLTVVEIMRAIHAGEIKGMFVEGENPAM
SDPDLNHARHALAMLDHLVVQDLFLTETAFHADVVLPASAFAEKAGTFTNTDRRVQISQPVVSP
PGDARQDWWIIQELGKPLGLPWNYGGPADIFREMAMVMPSFNNITWERLEREGAVTYPVDAPDK
PGNEIIFYAGFPTESGRAKIVPAAVVPPDELPDEDYPMVLSTGRVLEPWHTGSMTRRAGVLDAL
EPEAVAFMAPKELYRLGLEPGDTMKLETRRGAVHLKVRSDRDVPVGMIFMPFCYAEAAANLLIN
PALDPMGKIPEFKFCAARASAVHATPMAAE
-Exemplary Methylobacterium sp. XJLW Formate Dehydrogenase-N
Subunit Alpha (FDH5) Amino Acid Sequence
SEQ ID NO: 228
MTNLWMDIKHADVITVMGGNAAEAHPCGFKWVVEAKAHNNAKLIVVDPRFTRTASVADLYCPIR
QGTDIAFLSGVAKYLLDNDKLQHRYVSAYTNAGYVVREGYDFSEGLFAGYDADKRDYDKTTWDY
EIGPDGYAVVDETLQHPRCVMQLLKKHVALYTPEMVEKICGSPKDTFLKVCELIATTAAPDRVM
TSLYALGWTHHSKGSQNIRSMCIVQTLLGNIGMLGGGMNALRGHSNIQGLTDIGLMSNLIPGYL
NIPVEKEPDYASYIAKRQFKPLRPGQTSYWQNYNKFFVSFQKAMWGDKAQKENDWAYDYLPKLD
VPTYDVLRGFELAKQGKMTGYVIQGFNPLLSFPNRAKMTEAFSKMKFLVVMDPLKTETARFWEN
HGEYNDVDPTKIQTEVFELPTTLFVEEEGSLSNSSRWLQWHWQAQDAPGECRSDIEIMSEIFLR
IRGAYKKDGGAFSDPIVNLKWDYAIAESPTPTELARELNGYTLAPTPDLNGTVIPAGKQVDGFA
QLKDDGTTACGCWIYSGCYTEKGNMMARRDNTDPGDRGIAPNWAFAWPANRRVLYNRASCDPEG
RPWSEKKKLIEWNGKQWIGFDVPDYGVTVAPDKGVGPFILNQEGVARLWTRGLMRDGPFPTHYE
PFESPVQNVAFPKIKGAPAARIFKDDLADLGDAKDFPYAATSYRLTEHFHGWTKHARINAILQP
EAFVEISEELAKEKGIAKGGWVRVWSKRGSLKAKAVVTKRIKPLICDGKPVHVVGIPQHWGFMG
HTKKGWHPNSLTPVVGDANTETPEFKAWLVNIEPTTPPSDAVA
-Exemplary Methylobacterium sp. XJLW Formate Dehydrogenase-
Subunit Gamma (FDH6) Amino Acid Sequence
SEQ ID NO: 229
MARHEPWSAERASKIIAEHTHLEGATLPILHALQETFGYVDSGAVPLIADALNLSRAEVHGCIT
FYHDFRAHPAGRHEVKLCRAEACQAMGSDKLHREILGRLGCGWHETTADGSATVEPVYCLGLCA
NGPAALVDGEPVAHLTADALEAALTEVRQ
-Exemplary Methylobacterium sp. XJLW Formate Dehydrogenase-
Subunit Gamma (FDH7) Nucleic Acid Coding Sequence
SEQ ID NO: 230
ATGTACGTCCCGCGCTACACCGGCGTGCAGCGCGTGAACCACTGGATCACCGCGATCCTGTTCA
CGCTGCTGACCCTGTCGGGCCTGGCGATGTTCACGCCCTACCTGTTCTCGCTCACCGGCCTGTT
CGGTGGCGGGCAGGCGACCCGGGCGATCCATCCCTGGTTCGGCGTGGCGCTGGCGGTCAGCTTC
TTCTTCCTGTTCGTGCGCTTCTGGAAGCTCAACATCCCCAACAAGGACGATGTCGAGTGGACGA
AGCATATCGGCGACGTGGTCACCAACCGTGAGGACCGGCTCCCGGAGCTCGGCAAGTACAATGC
CGGACAGAAGGGCGTGTTCTGGGGGCAGACCGCGCTGATCGGCGTGATGTTCGTCACCGGGCTC
GTGATCTGGAACACCTATTTCGGCGGCCTCACCTCCATCGAGACCCAGCGCTGGGCGCTTCTGG
CCCACTCCCTCGCCGCGGTGATCGCCATCGCGATCATCGTGGTGCACATCTACGCCGGCATCTG
GGTCCGCGGCACCGGCCGGGCGATGGTCCGCGGCACGGTCACGGGCGGCTGGGCCTACCGCCAT
CACCGCAAGTGGTTCCGTCAGATGGCCGGCGGCACGGGCCGCCGGGGTTCGGTGGACAAGCGCG
GATCCTGA
-Exemplary Methylobacterium sp. XJLW Formate Dehydrogenase-
Subunit Gamma (FDH7) Amino Acid Sequence
SEQ ID NO: 231
MYVPRYTGVQRVNHWITAILFTLLTLSGLAMFTPYLFSLTGLFGGGQATRAIHPWFGVALAVSF
FFLFVRFWKLNIPNKDDVEWTKHIGDVVTNREDRLPELGKYNAGQKGVFWGQTALIGVMFVTGL
VIWNTYFGGLTSIETQRWALLAHSLAAVIAIAIIVVHIYAGIWVRGTGRAMVRGTVTGGWAYRH
HRKWFRQMAGGTGRRGSVDKRGS
-Exemplary Methylobacterium sp. XJLW Formate Dehydrogenase-
Subunit Beta (FDH8) Nucleic Acid Coding Sequence
SEQ ID NO: 232
ATGGCTGACTACAGCTCCCTCGACATCCGCCAGCGTTCCGCCTCCACGGAGACGCCGCCGGAGA
TCCGCCGCCAGGTGGAGGTCGCCAAGCTCATCGACGTGTCGAAGTGCATCGGCTGCAAGGCCTG
CCAATCGGCCTGCGAGGAGTGGAACGACCTCCGCGACGATATCGGCGTCAACACGGGCACGTAT
CAGAACCCCCACGACCTCACCCCGAAGTCGTGGACCCTGATGCGGTTCACCGAGTACGAGAACC
CCGAGACCCAGAACCTCGAATGGCTGATCCGCAAGGACGGCTGCATGCACTGCACCGAGCCGGG
CTGCCTGAAGGCCTGCCCGTCCCCCGGCGCCATCGTGCAGTACTCCAACGGCATCGTCGACTTC
ATCGAGGAGAACTGCATCGGCTGCGGCTATTGCGTGAAGGGTTGCCCCTTTAACATCCCGCGCA
TCAGCCAGACCGACCACAAGGCGTACAAGTGCACCCTGTGCTCGGACCGGGTGGCGGTGGGTCA
GGCTCCGGCCTGCGCCAAGGCCTGCCCGACCGGCTCGATCATGTTCGGCACCAAGCAGGCCATG
ATCGACCAGGCGCATGACCGCGTCGAGGATCTGAAGTCGCGCGGCTTCGCGCATGCCGGCCTCT
ACGACCCGGCCGGCGTCGGCGGCACGCACGTCATGTACGTGCTGCACCACGCCGACCAACCGAG
CCTCTACGCCGGTCTGCCGAACGACCCGAAGATCTCGCCGCTCGTCGCCTTCTGGAAGGGCGGA
GCGAAGGTGTTCGGTCTCGCTGCCATGGGCTTCGCCGCGGTGGCGGGCTTCTTCCACTACGTGA
CGGCCGGCCCCAACGAGGTCGTGCCCGAAGAGGAGGAAGAGGCGGTCGAATACGACGAGGCCAA
GCGCCGCGAGACCGGCGGCGGCGAGGCCAGGCCGCACTGA
-Exemplary Methylobacterium sp. XJLW Formate Dehydrogenase-
Subunit Beta (FDH8) Amino Acid Sequence
SEQ ID NO: 233
MADYSSLDIRQRSASTETPPEIRRQVEVAKLIDVSKCIGCKACQSACEEWNDLRDDIGVNTGTY
QNPHDLTPKSWTLMRFTEYENPETQNLEWLIRKDGCMHCTEPGCLKACPSPGAIVQYSNGIVDF
IEENCIGCGYCVKGCPFNIPRISQTDHKAYKCTLCSDRVAVGQAPACAKACPTGSIMFGTKQAM
IDQAHDRVEDLKSRGFAHAGLYDPAGVGGTHVMYVLHHADQPSLYAGLPNDPKISPLVAFWKGG
AKVFGLAAMGFAAVAGFFHYVTAGPNEVVPEEEEEAVEYDEAKRRETGGGEARPH
-Exemplary Pseudomonas putida Formate Dehydrogenase (FDHP)
Amino Acid Sequence
SEQ ID NO: 234
MAKVLCVLYDDPVDGYPKTYARDDLPKIDHYPGGQTLPTPKAIDFTPGQLLGSVSGELGLRKYL
ESNGHTLVVTSDKDGPDSVFERELVDADVVISQPFWPAYLTPERIAKAKNLKLALTAGIGSDHV
DLQSAIDRNVTVAEVTYCNSISVAEHVVMMILSLVRNYLPSHEWARKGGWNIADCVSHAYDLEA
MHVGTVAAGRIGLAVLRRLAPFDVHLHYTDRHRLPESVEKELNLTWHATREDMYPVCDVVTLNC
PLHPETEHMINDETLKLFKRGAYIVNTARGKLCDRDAVARALESGRLAGYAGDVWFPQPAPKDH
PWRTMPYNGMTPHISGTTLTAQARYAAGTREILEXFFEGRPIRDEYLIVQGGALAGTGAHSYSK
GNATGGSEEAAKFKKAV
-Exemplary Arabidopsis thaliana Formate Dehydrogenase (Chloroplastic
AtFDH1.1) Nucleic Acid Coding Sequence
SEQ ID NO: 235
ATGGCGATGAGACAAGCCGCTAAGGCAACGATCAGGGCCTGTTCTTCCTCTTCTTCTTCGGGTT
ACTTCGCTCGACGTCAGTTTAATGCATCTTCTGGTGATAGCAAAAAGATTGTAGGAGTTTTCTA
CAAGGCCAACGAATACGCTACCAAGAACCCTAACTTCCTTGGCTGCGTCGAGAATGCCTTAGGA
ATCCGTGACTGGCTTGAATCCCAAGGACATCAGTACATCGTCACTGATGACAAGGAAGGCCCTG
ATTGCGAACTTGAGAAACATATCCCGGATCTTCACGTCCTAATCTCCACTCCCTTCCACCCGGC
GTATGTAACTGCTGAAAGAATCAAGAAAGCCAAAAACTTGAAGCTTCTCCTCACAGCTGGTATT
GGCTCGGATCATATTGATCTCCAGGCAGCTGCAGCTGCTGGCCTGACGGTTGCTGAAGTCACGG
GAAGCAACGTGGTCTCAGTGGCAGAAGATGAGCTCATGAGAATCTTAATCCTCATGCGCAACTT
CGTACCAGGGTACAACCAGGTCGTCAAAGGCGAGTGGAACGTCGCGGGCATTGCGTACAGAGCT
TATGATCTTGAAGGGAAGACGATAGGAACCGTGGGAGCTGGAAGAATCGGAAAGCTTTTGCTGC
AGCGGTTGAAACCATTCGGGTGTAACTTGTTGTACCATGACAGGCTTCAGATGGCACCAGAGCT
GGAGAAAGAGACTGGAGCTAAGTTCGTTGAGGATCTGAATGAAATGCTCCCTAAATGTGACGTT
ATAGTCATCAACATGCCTCTCACGGAGAAGACAAGAGGAATGTTCAACAAAGAGTTGATAGGGA
AATTGAAGAAAGGCGTTTTGATAGTGAACAACGCAAGAGGAGCCATCATGGAGAGGCAAGCAGT
GGTGGATGCGGTGGAGAGTGGACACATTGGAGGGTACAGCGGAGACGTTTGGGACCCACAGCCA
GCTCCTAAGGACCATCCATGGCGTTACATGCCTAACCAGGCTATGACCCCTCATACCTCCGGCA
CCACCATTGACGCTCAGCTACGGTATGCGGGGGGACGAAAGACATGTTGGAGAGATACTTCAA
GGGAGAAGACTTCCCTACTGAGAATTACATCGTCAAGGACGGTGAACTTGCTCCTCAGTACCGG
TAA
-Exemplary Arabidopsis thaliana Formate Dehydrogenase (Chloroplastic
AtFDH1.1) Amino Acid Sequence
SEQ ID NO: 236
MAMRQAAKATIRACSSSSSSGYFARRQFNASSGDSKKIVGVFYKANEYATKNPNFLGCVENALG
IRDWLESQGHQYIVTDDKEGPDCELEKHIPDLHVLISTPFHPAYVTAERIKKAKNLKLLLTAGI
GSDHIDLQAAAAAGLTVAEVTGSNVVSVAEDELMRILILMRNFVPGYNQVVKGEWNVAGIAYRA
YDLEGKTIGTVGAGRIGKLLLQRLKPFGCNLLYHDRLQMAPELEKETGAKFVEDLNEMLPKCDV
IVINMPLTEKTRGMFNKELIGKLKKGVLIVNNARGAIMERQAVVDAVESGHIGGYSGDVWDPQP
APKDHPWRYMPNQAMTPHTSGTTIDAQLRYAAGTKDMLERYFKGEDFPTENYIVKDGELAPQYR
-Exemplary Arabidopsis thaliana Formate Dehydrogenase
(Mitochondrial AtFDH1.2) Nucleic Acid Coding Sequence
SEQ ID NO: 237
ATGATTTTTCAGAGTTTTAGCCTTTTGAACTTGCTTATGAAACAGGCATCTTCTGGTGATAGCA
AAAAGATTGTAGGAGTTTTCTACAAGGCCAACGAATACGCTACCAAGAACCCTAACTTCCTTGG
CTGCGTCGAGAATGCCTTAGGAATCCGTGACTGGCTTGAATCCCAAGGACATCAGTACATCGTC
ACTGATGACAAGGAAGGCCCTGATTGCGAACTTGAGAAACATATCCCGGATCTTCACGTCCTAA
TCTCCACTCCCTTCCACCCGGCGTATGTAACTGCTGAAAGAATCAAGAAAGCCAAAAACTTGAA
GCTTCTCCTCACAGCTGGTATTGGCTCGGATCATATTGATCTCCAGGCAGCTGCAGCTGCTGGC
CTGACGGTTGCTGAAGTCACGGGAAGCAACGTGGTCTCAGTGGCAGAAGATGAGCTCATGAGAA
TCTTAATCCTCATGCGCAACTTCGTACCAGGGTACAACCAGGTCGTCAAAGGCGAGTGGAACGT
CGCGGGCATTGCGTACAGAGCTTATGATCTTGAAGGGAAGACGATAGGAACCGTGGGAGCTGGA
AGAATCGGAAAGCTTTTGCTGCAGCGGTTGAAACCATTCGGGTGTAACTTGTTGTACCATGACA
GGCTTCAGATGGCACCAGAGCTGGAGAAAGAGACTGGAGCTAAGTTCGTTGAGGATCTGAATGA
AATGCTCCCTAAATGTGACGTTATAGTCATCAACATGCCTCTCACGGAGAAGACAAGAGGAATG
TTCAACAAAGAGTTGATAGGGAAATTGAAGAAAGGCGTTTTGATAGTGAACAACGCAAGAGGAG
CCATCATGGAGAGGCAAGCAGTGGTGGATGCGGTGGAGAGTGGACACATTGGAGGGTACAGCGG
AGACGTTTGGGACCCACAGCCAGCTCCTAAGGACCATCCATGGCGTTACATGCCTAACCAGGCT
ATGACCCCTCATACCTCCGGCACCACCATTGACGCTCAGCTACGGTATGCGGCGGGGACGAAAG
ACATGTTGGAGAGATACTTCAAGGGAGAAGACTTCCCTACTGAGAATTACATCGTCAAGGACGG
TGAACTTGCTCCTCAGTACCGGTAA
-Exemplary Arabidopsis thaliana Formate Dehydrogenase
(Mitochondrial AtFDH1.2) Amino Acid Sequence
SEQ ID NO: 238
MIFQSFSLLNLLMKQASSGDSKKIVGVFYKANEYATKNPNFLGCVENALGIRDWLESQGHQYIV
TDDKEGPDCELEKHIPDLHVLISTPFHPAYVTAERIKKAKNLKLLLTAGIGSDHIDLQAAAAAG
LTVAEVTGSNVVSVAEDELMRILILMRNFVPGYNQVVKGEWNVAGIAYRAYDLEGKTIGTVGAG
RIGKLLLQRLKPFGCNLLYHDRLQMAPELEKETGAKFVEDLNEMLPKCDVIVINMPLTEKTRGM
FNKELIGKLKKGVLIVNNARGAIMERQAVVDAVESGHIGGYSGDVWDPQPAPKDHPWRYMPNQA
MTPHTSGTTIDAQLRYAAGTKDMLERYFKGEDFPTENYIVKDGELAPQYR
-Exemplary Arabidopsis thaliana Formate Dehydrogenase (AtFDH1.3)
Nucleic Acid Coding Sequence
SEQ ID NO: 239
ATGAAACAAGCCAGTTCAGGCGATTCAAAAAAGATAGTCGGGGTGTTTTATAAAGCTAACGAGT
ACGCCACAAAGAATCCAAACTTTCTTGGCTGCGTCGAAAACGCTCTTGGGATACGGGATTGGCT
CGAATCCCAAGGTCATCAATATATTGTGACAGATGACAAGGAAGGTCCCGATTGTGAATTAGAG
AAACATATTCCCGATTTACATGTATTGATATCAACACCCTTTCACCCCGCCTATGTAACTGCTG
AGAGGATTAAAAAGGCCAAAAATTTGAAACTCCTATTGACTGCCGGGATAGGATCAGACCACAT
AGATTTACAAGCCGCTGCAGCCGCTGGGCTGACAGTCGCGGAGGTGACGGGATCCAACGTTGTA
TCTGTAGCCGAGGATGAGCTCATGAGAATACTGATCTTAATGCGGAACTTTGTACCTGGATATA
ATCAAGTAGTTAAGGGTGAGTGGAATGTTGCGGGTATTGCCTATAGAGCATACGACTTAGAGGG
GAAAACGATCGGTACCGTGGGCGCCGGGCGTATTGGTAAATTACTTCTGCAAAGACTTAAACCC
TTTGGGTGTAATCTACTCTATCACGATAGACTTCAGATGGCACCCGAATTGGAAAAAGAGACTG
GAGCGAAATTCGTAGAGGACCTTAATGAAATGTTACCTAAATGCGACGTAATAGTCATTAATAT
GCCCCTAACCGAAAAAACTAGAGGTATGTTTAACAAAGAACTCATCGGTAAGTTAAAAAAGGGC
GTCTTGATTGTTAATAACGCCCGAGGAGCTATCATGGAGCGCCAAGCCGTTGTCGACGCTGTAG
AAAGTGGACACATTGGCGGGTATTCTGGGGATGTCTGGGATCCCCAACCAGCTCCTAAGGATCA
TCCTTGGCGGTACATGCCAAATCAAGCCATGACACCTCATACATCCGGCACCACTATAGATGCA
CAATTACGATATGCCGCTGGCACAAAAGATATGCTTGAACGGTATTTTAAGGGAGAGGACTTTC
CCACAGAAAATTATATTGTAAAGGATGGGGAGTTGGCTCCCCAGTATAGATAA
-Exemplary Arabidopsis thaliana Formate Dehydrogenase (AtFDH1.3)
Amino Acid Sequence
SEQ ID NO: 240
MKQASSGDSKKIVGVFYKANEYATKNPNFLGCVENALGIRDWLESQGHQYIVTDDKEGPDCELE
KHIPDLHVLISTPFHPAYVTAERIKKAKNLKLLLTAGIGSDHIDLQAAAAAGLTVAEVTGSNVV
SVAEDELMRILILMRNFVPGYNQVVKGEWNVAGIAYRAYDLEGKTIGTVGAGRIGKLLLQRLKP
FGCNLLYHDRLQMAPELEKETGAKFVEDLNEMLPKCDVIVINMPLTEKTRGMENKELIGKLKKG
VLIVNNARGAIMERQAVVDAVESGHIGGYSGDVWDPQPAPKDHPWRYMPNQAMTPHTSGITIDA
QLRYAAGTKDMLERYFKGEDFPTENYIVKDGELAPQYR

### Serine Hydroxymethyltransferase 1, Mitochondrial (SHM1)

In certain embodiments, a composition described herein comprises at least one transgenic SHM1 enzyme. In some embodiments, SHM1 enzymes catalyze the interconversion of serine and glycine.

In some embodiments, a SHM1 gene and/or transgene comprises a sequence encoding a peptide sequence that is at least 90%, 91%, 92%, 93%, 94%, 95%, 96%, 97%, 98%, 99%, or 100% identical to SEQ ID NO: 404 (or a portion thereof). In some embodiments, a FDH gene and/or transgene comprises a nucleotide sequence that is at least 90%, 91%, 92%, 93%, 94%, 95%, 96%, 97%, 98%, 99%, or 100% identical to SEQ ID NO: 403 (or a portion thereof).

-Exemplary Arabidopsis thaliana Serine hydroxymethyltransferase 1,
mitochondrial (SHM1) Nucleic Acid Coding Sequence
SEQ ID NO: 403
ATGGCGATGGCCATGGCTCTTCGAAGGCTTTCTTCTTCAATTGACAAACCCATTCGTCCTCTTA
TTCGATCCACTTCATGTTACATGTCTTCTTTGCCCAGTGAAGCTGTTGATGAGAAGGAAAGATC
TCGTGTCACTTGGCCAAAACAGCTTAACGCACCTTTAGAGGAGGTTGATCCTGAGATTGCTGAC
ATTATTGAGCATGAGAAAGCTAGACAATGGAAGGGACTTGAACTTATTCCATCTGAGAACTTCA
CATCTGTGTCGGTGATGCAAGCTGTTGGGTCTGTCATGACTAACAAATACAGTGAAGGCTATCC
TGGTGCCAGATACTATGGAGGAAATGAGTATATAGACATGGCAGAAACCTTATGCCAGAAGCGC
GCTCTTGAAGCTTTCCGGTTAGATCCTGAAAAGTGGGGAGTGAATGTTCAACCTTTGTCTGGAT
CTCCTGCCAACTTCCATGTGTACACTGCATTGTTAAAGCCTCATGAAAGAATCATGGCACTTGA
TCTTCCTCATGGTGGTCATCTTTCTCATGGTTATCAGACTGACACCAAGAAGATATCAGCTGTG
TCTATCTTCTTTGAAACAATGCCCTATAGATTGGACGAGAGCACTGGCTACATCGACTACGATC
AGATGGAGAAAAGTGCTACTCTTTTCAGGCCAAAATTGATTGTTGCTGGTGCAAGTGCTTATGC
TAGATTGTATGACTATGCCCGCATCAGAAAGGTCTGTAACAAGCAAAAAGCTGTAATGCTAGCA
GATATGGCACACATCAGTGGTTTGGTTGCTGCTAATGTAATCCCTTCACCGTTCGACTATGCTG
ATGTTGTAACCACCACAACTCACAAGTCACTTCGTGGACCCCGTGGAGCCATGATTTTCTTCAG
AAAGGGTGTTAAGGAAATTAACAAGCAAGGGAAAGAGGTTTTGTATGATTTTGAAGACAAGATC
AACCAAGCTGTCTTCCCTGGTCTTCAAGGTGGTCCACACAACCACACTATCACAGGACTAGCTG
TTGCTTTGAAACAGGCAACTACTTCAGAGTACAAAGCATACCAAGAACAAGTCCTGAGTAACAG
TGCAAAGTTTGCTCAGACTCTAATGGAGAGAGGATATGAACTTGTTTCTGGTGGAACTGACAAC
CATCTGGTTCTAGTGAATCTAAAGCCCAAGGGAATTGATGGATCTAGAGTTGAGAAAGTGTTGG
AAGCTGTTCACATTGCATCCAACAAAAACACTGTTCCTGGAGATGTTTCTGCCATGGTTCCTGG
TGGAATCAGAATGGGTACTCCTGCTCTCACTTCCAGAGGCTTTGTTGAGGAAGACTTTGCCAAA
GTAGCTGAATACTTCGACAAAGCTGTGACAATAGCTCTCAAAGTCAAATCTGAAGCTCAAGGAA
CCAAGTTGAAGGATTTCGTGTCAGCAATGGAATCCTCTTCAACCATCCAATCCGAGATTGCGAA
ACTGCGCCATGAAGTCGAGGAATTCGCTAAGCAGTTCCCAACAATTGGGTTTGAGAAAGAAACC
ATGAAGTACAAGAACTAA
-Exemplary Arabidopsis thaliana Serine hydroxymethyltransferase 1,
mitochondrial (SHM1) Amino Acid Sequence
SEQ ID NO: 404
MAMAMALRRLSSSIDKPIRPLIRSTSCYMSSLPSEAVDEKERSRVTWPKQLNAPLEEVDPEIAD
IIEHEKARQWKGLELIPSENFTSVSVMQAVGSVMINKYSEGYPGARYYGGNEYIDMAETLCQKR
ALEAFRLDPEKWGVNVQPLSGSPANFHVYTALLKPHERIMALDLPHGGHLSHGYQTDTKKISAV
SIFFETMPYRLDESTGYIDYDQMEKSATLFRPKLIVAGASAYARLYDYARIRKVCNKQKAVMLA
DMAHISGLVAANVIPSPFDYADVVTTTTHKSLRGPRGAMIFFRKGVKEINKQGKEVLYDFEDKI
NQAVFPGLQGGPHNHTITGLAVALKQATTSEYKAYQEQVLSNSAKFAQTLMERGYELVSGGTDN
HLVLVNLKPKGIDGSRVEKVLEAVHIASNKNTVPGDVSAMVPGGIRMGTPALTSRGFVEEDFAK
VAEYFDKAVTIALKVKSEAQGTKLKDFVSAMESSSTIQSEIAKLRHEVEEFAKQFPTIGFEKET
MKYKN

### (S)-2-Hydroxy-Acid Oxidase (GLO)

In certain embodiments, a composition described herein comprises at least one transgenic GLO1 and/or GLO2 enzyme. In some embodiments, GLO enzymes catalyze the interconversion of (2S)-2-hydroxycarboxylate and 2-oxocarboxylate.

In some embodiments, a GLO gene and/or transgene comprises a sequence encoding a peptide sequence that is at least 90%, 91%, 92%, 93%, 94%, 95%, 96%, 97%, 98%, 99%, or 100% identical to SEQ ID NO: 406 or 408 (or a portion thereof). In some embodiments, a FDH gene and/or transgene comprises a nucleotide sequence that is at least 90%, 91%, 92%, 93%, 94%, 95%, 96%, 97%, 98%, 99%, or 100% identical to SEQ ID NO: 405 or 407 (or a portion thereof).

-Exemplary Arabidopsis thaliana (S)-2-hydroxy-acid oxidase (GLO1)
Nucleic Acid Coding Sequence
SEQ ID NO: 405
ATGGAGATCACTAACGTTACCGAGTATGATGCAATCGCAAAGCAGAAGCTGCCTAAGATGGTGT
ACGACTACTATGCATCTGGTGCAGAAGACCAATGGACTCTTCAAGAGAACAGAAACGCTTTTGC
AAGGATCCTCTTTCGGCCTCGGATTCTGATTGATGTGAGCAAGATTGACATGACAACCACCGTC
TTGGGGTTCAAGATCTCGATGCCCATCATGGTTGCTCCAACTGCCATGCAAAAGATGGCTCACC
CTGATGGGGAATATGCTACTGCTAGAGCTGCATCTGCAGCTGGAACTATCATGACACTATCTTC
ATGGGCTACTTCCAGCGTTGAAGAAGTTGCGTCTACAGGGCCAGGGATCCGATTCTTCCAGCTC
TATGTATACAAGAACAGGAATGTGGTTGAGCAGCTCGTGAGAAGAGCTGAGAGGGCTGGGTTCA
AAGCCATTGCTCTCACTGTAGACACCCCAAGGCTAGGCCGCAGAGAGTCTGATATCAAGAACAG
ATTCACTTTGCCTCCAAACCTGACATTGAAGAACTTTGAAGGACTTGACCTCGGAAAGATGGAC
GAGGCCAATGACTCTGGCTTGGCTTCATATGTTGCTGGTCAAATTGACCGTACCTTAAGCTGGA
AGGATGTCCAGTGGCTCCAGACAATCACCAAGTTGCCCATTCTTGTCAAAGGTGTTCTTACAGG
AGAGGATGCAAGGATAGCGATTCAAGCTGGTGCAGCCGGAATCATTGTATCAAACCATGGAGCT
CGCCAGCTTGACTATGTCCCAGCAACCATCTCGGCCCTTGAAGAGGTTGTCAAAGCGACACAAG
GACGAATTCCTGTCTTCTTGGATGGTGGTGTTCGACGTGGCACTGATGTCTTCAAAGCACTTGC
ACTTGGAGCCTCCGGGATATTTATTGGAAGACCAGTGGTATTCTCATTGGCAGCTGAAGGAGAG
GCTGGAGTTAGAAAGGTGCTTCAAATGCTACGTGATGAGTTCGAGCTGACCATGGCACTGAGTG
GGTGTCGGTCCCTAAAGGAAATCTCCCGTAACCACATTACCACCGAATGGGACACTCCACGTCC
TTCAGCCAGGTTATAG
-Exemplary Arabidopsis thaliana (S)-2-hydroxy-acid oxidase (GLO1)
Amino Acid Sequence
SEQ ID NO: 406
MEITNVTEYDAIAKQKLPKMVYDYYASGAEDQWTLQENRNAFARILFRPRILIDVSKIDMTTTV
LGFKISMPIMVAPTAMQKMAHPDGEYATARAASAAGTIMTLSSWATSSVEEVASTGPGIRFFQL
YVYKNRNVVEQLVRRAERAGFKAIALTVDTPRLGRRESDIKNRFTLPPNLTLKNFEGLDLGKMD
EANDSGLASYVAGQIDRTLSWKDVQWLQTITKLPILVKGVLIGEDARIAIQAGAAGIIVSNHGA
RQLDYVPATISALEEVVKATQGRIPVFLDGGVRRGTDVFKALALGASGIFIGRPVVFSLAAEGE
AGVRKVLQMLRDEFELTMALSGCRSLKEISRNHITTEWDTPRPSARL
-Exemplary Arabidopsis thaliana (S)-2-hydroxy-acid oxidase (GLO2)
Nucleic Acid Coding Sequence
SEQ ID NO: 407
ATGGAGATCACTAACGTTACCGAGTATGATGCAATCGCAAAGGCGAAGTTGCCTAAGATGGTAT
ATGACTACTATGCATCTGGTGCAGAAGATCAATGGACTCTTCAAGAGAACAGAAACGCTTTTGC
AAGAATCCTCTTCCGGCCTCGGATTTTGATTGATGTGAACAAAATTGATATGGCGACTACCGTC
TTGGGGTTCAAGATCTCGATGCCGATCATGGTTGCTCCTACTGCCTTTCAAAAGATGGCTCACC
CTGATGGGGAATATGCTACGGCTAGAGCTGCGTCTGCTGCTGGAACCATCATGACACTATCTTC
ATGGGCTACTTCAAGTGTTGAAGAAGTTGCTTCCACAGGGCCAGGAATCCGATTCTTCCAGCTC
TATGTATACAAGAACAGGAAGGTGGTTGAGCAGCTCGTGAGAAGAGCCGAGAAAGCTGGGTTCA
AAGCCATTGCTCTCACTGTAGACACCCCAAGGCTAGGTCGCAGAGAGTCTGATATCAAGAACAG
ATTCACTTTGCCTCCAAACCTGACATTGAAGAACTTTGAAGGTCTTGACCTTGGAAAGATGGAC
GAGGCCAATGACTCTGGCTTGGCTTCGTATGTTGCTGGTCAAATTGACCGTACCTTGAGCTGGA
AGGATATCCAGTGGCTCCAAACAATCACCAACATGCCAATTCTTGTCAAGGGTGTTCTTACAGG
AGAGGATGCAAGGATAGCGATTCAAGCTGGAGCAGCAGGGATCATTGTGTCAAATCATGGAGCT
CGCCAGCTTGATTATGTCCCAGCAACAATCTCAGCCCTTGAAGAGGTTGTCAAAGCAACACAAG
GACGAGTTCCTGTCTTCTTGGATGGTGGTGTTCGACGTGGCACTGATGTCTTCAAGGCACTTGC
ACTTGGAGCCTCTGGAATATTTATTGGAAGACCAGTGGTTTTTGCACTAGCTGCTGAAGGAGAA
GCCGGAGTCAAAAAGGTGCTTCAAATGTTGCGTGATGAGTTCGAGCTAACCATGGCACTAAGTG
GGTGCCGGTCACTCAGTGAAATCACCCGTAACCACATTGTCACGGAATGGGACACTCCACGCCA
TTTGCCCAGGTTATAG
-Exemplary Arabidopsis thaliana (S)-2-hydroxy-acid oxidase (GLO2)
Amino Acid Sequence
SEQ ID NO: 408
MEITNVTEYDAIAKAKLPKMVYDYYASGAEDQWTLQENRNAFARILFRPRILIDVNKIDMATTV
LGFKISMPIMVAPTAFQKMAHPDGEYATARAASAAGTIMTLSSWATSSVEEVASTGPGIRFFQL
YVYKNRKVVEQLVRRAEKAGFKAIALTVDTPRLGRRESDIKNRFTLPPNLTLKNFEGLDLGKMD
EANDSGLASYVAGQIDRTLSWKDIQWLQTITNMPILVKGVLTGEDARIAIQAGAAGIIVSNHGA
RQLDYVPATISALEEVVKATQGRVPVFLDGGVRRGTDVFKALALGASGIFIGRPVVFALAAEGE
AGVKKVLQMLRDEFELTMALSGCRSLSEITRNHIVTEWDTPRHLPRL

### F) Homoserine Pathway

In some embodiments, compositions and methods described herein comprise introduction of one or more genes coding for one or more enzymes involved in the metabolism of HCHO to act as a carbon source to synthesize homoserine. In some embodiments of such a metabolic pathway, HCHO may be metabolized through the following metabolic mechanism (pathway 7): 1) serine aldolase (SAL) or threonine aldolase (LtaE) combining HOCH with glycine to form serine 2) serine being then deaminated to pyruvate by serine deaminase (SDA) 3) 4-hydroxy-2-oxobutanoate (HOB) aldolase (HAL) combining formaldehyde and pyruvate to from HOB 4) HOB aminotransferase (HAT) turning HOB into homoserine 5) homoserine (HSer) integrating various endogenous plant metabolic pathways. In certain embodiments, one or more of the enzymatic components of this pathway may be introduced as a transgene as described herein (see FIGS. 4-9).

### Serine Aldolase (SAL) or Threonine Aldolase (LtaE)

In some embodiments, a composition described herein comprises a transgenic SAL and/or LtaE protein. In some embodiments, such a protein, among other things, may utilize formaldehyde as a substrate and produce serine.

In some embodiments, a SAL or LtaE gene and/or transgene comprises a sequence encoding a peptide sequence that is at least 90%, 91%, 92%, 93%, 94%, 95%, 96%, 97%, 98%, 99%, or 100% identical to SEQ ID NO: 241 (or a portion thereof).

-Exemplary Escherichia coli Serine Aldolase and/or Threonine aldolase
(SAL and/or LtaE) Amino Acid Sequence
SEQ ID NO: 241
MIDLRSDTVTRPSRAMLEAMMAAPVGDDVYGDDPTVNALQDYAAELSGKEAAIFLPTGTQANLV
ALLSHCERGEEYIVGQAAHNYLFEAGGAAVLGSIQPQPIDAAADGTLPLDKVAMKIKPDDIHFA
RTKLLSLENTHNGKVLPREYLKEAWEFTRERNLALHVDGARIFNAVVAYGCELKEITQYCDSFT
ICLSKGLGTPVGSLLVGNRDYIKRAIRWRKMTGGGMRQSGILAAAGIYALKNNVARLQEDHDNA
AWMAEQLREAGADVMRQDTNMLFVRVGEENAAALGEYMKARNVLINASPIVRLVTHLDVSREQL
AEVAAHWRAFLAR

Serine Deaminase (sdaA)

In some embodiments, a composition described herein comprises a transgenic sdaA protein. In some embodiments, such a protein, among other things, may utilize serine as a substrate and produce pyruvate.

In some embodiments, a sdaA gene and/or transgene comprises a sequence encoding a peptide sequence that is at least 90%, 91%, 92%, 93%, 94%, 95%, 96%, 97%, 98%, 99%, or 100% identical to SEQ ID NO: 242 (or a portion thereof).

-Exemplary Escherichia coli Serine Deaminase (sdaA) Amino Acid
Sequence
SEQ ID NO: 242
MISLFDMFKVGIGPSSSHTVGPMKAGKQFVDDLVEKGLLDSVTRVAVDVYGSLSLTGKGHHTDI
AIIMGLAGNEPATVDIDSIPGFIRDVEERERLLLAQGRHEVDFPRDNGMRFHNGNLPLHENGMQ
IHAYNGDEVVYSKTYYSIGGGFIVDEEHFGQDAANEVSVPYPFKSATELLAYCNETGYSLSGLA
MQNELALHSKKEIDEYFAHVWQTMQACIDRGMNTEGVLPGPLRVPRRASALRRMLVSSDKLSND
PMNVIDWVNMFALAVNEENAAGGRVVTAPTNGACGIVPAVLAYYDHFIESVSPDIYTRYFMAAG
AIGALYKMNASISGAEVGCQGEVGVACSMAAAGLAELLGGSPEQVCVAAEIGMEHNLGLTCDPV
AGQVQVPCIERNAIASVKAINAARMALRRTSAPRVSLDKVIETMYETGKDMNAKYRETSRGGLA
IKVQCD

### 4-Hydroxy-2-Oxobutanoate (HOB) Aldolase (HAL)

In some embodiments, a composition described herein comprises a transgenic HAL protein. In some embodiments, such a protein, among other things, may utilize pyruvate and HCHO substrates and produce 4-hydroxy-2-oxobutanoate.

In some embodiments, a HAL gene and/or transgene comprises a sequence encoding a peptide sequence that is at least 90%, 91%, 92%, 93%, 94%, 95%, 96%, 97%, 98%, 99%, or 100% identical to SEQ ID NO: 243 (or a portion thereof).

-Exemplary Escherichia coli 4-hydroxy-2-oxobutanoate Aldolase (HAL)
Amino Acid Sequence
SEQ ID NO: 243
MNALLSNPFKERLRKGEVQIGLWLSSTTAYMAEIAATSGYDWLLIDGEHAPNTIQDLYHQLQAV
APYASQPVIRPVEGSKPLIKQVLDIGAQTLLIPMVDTAEQARQVVSATRYPPYGERGVGASVAR
AARWGRIENYMAQVNDSLCLLVQVESKTALDNLDEILDVEGIDGVFIGPADLSASLGYPDNAGH
PEVQRIIETSIRRIRAAGKAAGFLAVAPDMAQQCLAWGANFVAVGVDTMLYSDALDQRLAMFKS
GKNGPRIKGSY

### HOB Aminotransferase (HAT)

In some embodiments, a composition described herein comprises a transgenic HAT protein. In some embodiments, such a protein, among other things, may HOB as a substrate and produce homoserine.

In some embodiments, a HAT gene and/or transgene comprises a sequence encoding a peptide sequence that is at least 90%, 91%, 92%, 93%, 94%, 95%, 96%, 97%, 98%, 99%, or 100% identical to SEQ ID NO: 244 (or a portion thereof).

-Exemplary Escherichia coli 4-hydroxy-2-oxobutanoate Aldolase (HAL)
Amino Acid Sequence
SEQ ID NO: 244
MFENITAAPADPILGLADLFRADERPGKINLGIGVYKDETGKTPVLTSVKKAEQYLLENETTKN
YLGIDGIPEFGRCTQELLFGKGSALINDKRARTAQTPGGTGALRVAADFLAKNTSVKRVWVSNP
SWPNHKSVENSAGLEVREYAYYDAENHTLDFDALINSLNEAQAGDVVLFHGCCHNPTGIDPTLE
QWQTLAQLSVEKGWLPLFDFAYQGFARGLEEDAEGLRAFAAMHKELIVASSYSKNFGLYNERVG
ACTLVAADSETVDRAFSQMKAAIRANYSNPPAHGASVVATILSNDALRAIWEQELTDMRQRIQR
MRQLFVNTLQEKGANRDFSFIIKQNGMFSFSGLTKEQVLRLREEFGVYAVASGRVNVAGMTPDN
MAPLCEAIVAVL

### G) Formolase Pathway

In some embodiments, the present disclosure provides compositions comprising novel combinations of species and metabolic pathways. In some embodiments, a “Formolase pathway” can be introduced into an ornamental plant species. Formolase, was recently engineered through a combination of computational protein design and directed evolution. Mass spectrometry revealed that the engineered enzyme produces two products of the formose reaction-dihydroxyacetone and glycolaldehyde—with the product profile dependent on the formaldehyde concentration (see e.g., Poust et al., Mechanistic Analysis of an Engineered Enzyme that Catalyzes the Formose Reaction, ChemBioChem 2015; which is incorporated herein by reference in its entirety). The formolase couples formaldehyde to form glycolaldehyde and dihydroxyacetone (DHA). At high formaldehyde concentrations DHA is the primary product, whereas at low formaldehyde concentrations glycoaldehyde is the primary product. In some embodiments, the formolase pathway, consisting of a small number of thermodynamically favorable chemical transformations that convert formate into a three-carbon sugar in central metabolism (see e.g. Siegel et al., Computational protein design enables a novel one-carbon assimilation pathway. PNAS 2015; which is incorporated herein by reference in its entirety). When supplemented with enzymes carrying out the other steps in the pathway, Formolase converts formate into dihydroxyacetone phosphate and other central metabolites in vitro. Unlike native carbon fixation pathways, this pathway is linear, not oxygen sensitive, and consists of a small number of thermodynamically favorable steps.

In certain embodiments, Formolase is a synthetic enzyme that uptakes 3 molecules of formaldehyde to produce DHA. In certain embodiments, if Formolase is combined with DAK, it can be used as an alternative to DAS, which only uptakes 1 formaldehyde for each DHA produced.

### BTEX Metabolism

In certain embodiments, the present disclosure provides compositions and methods suited for the relatively efficient biodegradation of benzene, toluene, ethylbenzene, and xylene. In certain embodiments, following ring cleavage, benzene and toluene can enter the Calvin cycle where they may be converted to organic molecules and/or amino acids. In some embodiments, a pathway that is engineered is described in FIG. 3.

Benzene and Ethylbenzene: In some embodiments, benzene and/or ethylbenzene can be remediated through the actions of transgenes encoding enzymes such as but not limited to: benzene 1,2-dioxygenase and/or cis-1,2-dihydrobenzene-1,2-diol dehydrogenase.

Toluene and Xylene: In some embodiments, the phytoremediation of these two pollutants can be enhanced through the addition of a pathway comprising, but not limited to, genes coding for toluene methyl-monooxygenase, aryl-alcohol dehydrogenase, benzaldehyde dehydrogenase (NAD+) and/or benzaldehyde dehydrogenase (NADP+).

### Benzene, Toluene, Ethylbenzene, and Xylene (BTEX) Metabolizing Enzymes

In certain embodiments, a composition described herein comprises at least one transgenic BTEX metabolizing enzyme. In certain embodiments, exemplary BTEX metabolizing proteins utilize substrates such as benzene, toluene, ethylbenzene, and/or xylene to produce intermediate metabolic products such as phenol and/or phenol (like).

In some embodiments, a BTEX metabolizing gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 246, 248, 250, 252, 254, 256, 258, 260, or 262 (or a portion thereof). In some embodiments, a BTEX metabolizing gene and/or transgene comprises a nucleotide sequence that is at least 90%, 91%, 92%, 93%, 94%, 95%, 96%, 97%, 98%, 99%, or 100% identical to any one of SEQ ID NOs: 245, 247, 249, 251, 253, 255, 257, 259, or 261 (or a portion thereof).

-Exemplary Rhodococcus ruber cytochrome P450 monooxygenase
(P450-RR) Nucleic Acid Coding Sequence
SEQ ID NO: 245
ATGAGTGCATCAGTTCCGGCGTCGGCGTGTCCCGTCGATCACGCGGCCCTGGCCGGCGGCTGTC
CGGTGTCGACGAACGCCGCGGCGTTCGATCCGTTCGGGCCCGCGTACCAGGCCGATCCGGCCGA
GTCGCTGCGCTGGTCCCGCGACGAGGAGCCGGTGTTCTACAGCCCCGAACTCGGCTACTGGGTG
GTCACCCGCTACGAGGATGTGAAGGCGGTGTTCCGCGACAACCTCGTGTTCTCACCGGCCATCG
CCCTCGAGAAGATCACCCCGGTCTCCGAGGAGGCCACCGCCACCCTCGCCCGCTACGACTACGC
CATGGCCCGGACCCTCGTGAACGAGGACGAGCCCGCCCACATGCCGCGCCGCCGCGCACTCATG
GACCCGTTCACCCCGAAGGAACTGGCGCACCACGAGGCGATGGTGCGACGGCTCACGCGCGAAT
ACGTCGACCGCTTCGTCGAATCCGGCAAGGCCGACCTGGTGGACGAGATGCTGTGGGAGGTACC
GCTCACCGTCGCCCTGCACTTCCTCGGCGTGCCGGAGGAGGACATGGCGACGATGCGCAAGTAC
TCGATCGCCCACACCGTGAACACCTGGGGCCGCCCCGCGCCCGAGGAGCAGGTCGCCGTCGCCG
AGGCGGTCGGCAGGTTCTGGCAGTACGCGGGCACGGTGCTCGAGAAGATGCGCCAGGACCCCTC
GGGGCACGGCTGGATGCCCTACGGGATCCGCATGCAGCAGCAGATGCCGGACGTCGTCACCGAC
TCCTACCTGCACTCGATGATGATGGCCGGCATCGTCGCCGCGCACGAGACCACGGCCAACGCGT
CCGCGAACGCGTTCAAGCTGCTGCTCGAGAACCGCCCGGTGTGGGAGGAGATCTGCGCGGATCC
GTCGCTGATCCCCAACGCCGTCGAGGAGTGCCTGCGCCACTCGGGATCGGTCGCGGCGTGGCGA
CGGGTGGCCACCACCGACACCCGCATCGGCGACGTCGACATCCCCGCCGGCGCAAAGCTGCTCG
TCGTCAACGCCTCCGCCAACCATGACGAGCGGCACTTCGACCGTCCCGACGAGTTCGACATCCG
GCGCCCGAACTCGAGCGACCACCTCACCTTCGGGTACGGCAGCCATCAGTGCATGGGCAAGAAC
CTGGCCCGCATGGAGATGCAGATCTTCCTCGAGGAACTGACCACGCGGCTTCCCCACATGGAAC
TCGTACCCGATCAGGAGTTCACCTACCTGCCGAACACCTCGTTCCGCGGTCCCGATCACGTGTG
GGTGCAGTGGGATCCGCAGGCGAACCCCGAGCGCACCGACCCGGCCGTGCTGCAACGGCAGCAT
CCCGTCACCATCGGCGAGCCCTCCACCCGGTCGGTGTCACGCACCGTCACCGTCGAGCGCCTGG
ACCGGATCGTCGACGACGTGCTGCGCGTCGTCCTACGGGCTCCTGCAGGAAATGCGTTGCCCGC
GTGGACTCCTGGCGCCCACATCGATGTCGACCTCGGTGCGCTGTCGCGGCAGTACTCCCTGTGC
GGTGCGCCCGACGCGCCCACCTACGAGATCGCCGTTCTGCTGGACCCCGAGAGCCGCGGTGGCT
CGCGCTACGTCCACGAACAGCTCCGGGTGGGGGGATCGCTCCGGATTCGCGGGCCCCGGAACCA
CTTCGCGCTCGACCCCGACGCCGAGCACTACGTGTTCGTGGCCGGCGGCATCGGCATCACCCCC
GTCCTGGCCATGGCCGACCACGCCCGCGCCCGGGGGTGGAGCTACGAACTGCACTACTGCGGCC
GGAACCGTTCCGGGATGGCCTATCTCGAGCGGGTCGCCGGGCACGGGGACCGCGCCGCCCTGCA
CGTCTCGGCGGAAGGCACCCGGGTCGACCTCGCCGCCCTCCTCGCGACGCCGGTGTCCGGCACC
CAGATCTACGCGTGCGGGCCCGGACGGCTGCTCGCCGGACTCGAGGACGCGAGCCGGCACTGGC
CCGACGGTGCGCTGCACGTCGAGCACTTCACCTCGTCCCTCACGGCACTCGACCCGGACGTCGA
GCACGCCTTCGACCTCGACCTGCGCGACTCGGGACTCACCGTGCGGGTCGAGCCCACCCAGACC
GTCCTCGACGCGTTGCGCGCCAACAACATCGACGTGCCCAGCGACTGCGAGGAAGGCCTCTGCG
GCTCCTGCGAGGTCACCGTCCTCGAAGGCGAGGTCGACCACCGCGACACCGTGCTCACCAAGGC
CGAGCGGGCGGCGAACCGGCAGATGATGACCTGCTGCTCGCGTGCCTGCGGCGACCGACTGACC
CTCCGACTCTGA
-Exemplary Rhodococcus ruber cytochrome P450 monooxygenase (P450-RR)
Amino Acid Sequence
SEQ ID NO: 246
MSASVPASACPVDHAALAGGCPVSTNAAAFDPFGPAYQADPAESLRWSRDEEPVFYSPELGYWV
VTRYEDVKAVFRDNLVFSPAIALEKITPVSEEATATLARYDYAMARTLVNEDEPAHMPRRRALM
DPFTPKELAHHEAMVRRLTREYVDRFVESGKADLVDEMLWEVPLTVALHFLGVPEEDMATMRKY
SIAHTVNTWGRPAPEEQVAVAEAVGRFWQYAGTVLEKMRQDPSGHGWMPYGIRMQQQMPDVVTD
SYLHSMMMAGIVAAHETTANASANAFKLLLENRPVWEEICADPSLIPNAVEECLRHSGSVAAWR
RVATTDTRIGDVDIPAGAKLLVVNASANHDERHFDRPDEFDIRRPNSSDHLTFGYGSHQCMGKN
LARMEMQIFLEELTTRLPHMELVPDQEFTYLPNTSFRGPDHVWVQWDPQANPERTDPAVLQRQH
PVTIGEPSTRSVSRTVTVERLDRIVDDVLRVVLRAPAGNALPAWTPGAHIDVDLGALSRQYSLC
GAPDAPTYEIAVLLDPESRGGSRYVHEQLRVGGSLRIRGPRNHFALDPDAEHYVFVAGGIGITP
VLAMADHARARGWSYELHYCGRNRSGMAYLERVAGHGDRAALHVSAEGTRVDLAALLATPVSGT
QIYACGPGRLLAGLEDASRHWPDGALHVEHFTSSLTALDPDVEHAFDLDLRDSGLTVRVEPTQT
VLDALRANNIDVPSDCEEGLCGSCEVTVLEGEVDHRDTVLTKAERAANRQMMTCCSRACGDRLT
LRL
-Exemplary Pseudomonas stutzeri Toluene, O-xylene monooxygenase
oxygenase subunit alpha (TouA-P-sp-OX) Nucleic Acid Coding Sequence
SEQ ID NO: 247
ATGTCCATGCTGAAGAGAGAAGATTGGTATGACCTTACAAGGACAACTAACTGGACACCTAAGT
ACGTTACCGAGAATGAACTCTTTCCTGAGGAGATGTCAGGAGCAAGGGGAATTTCAATGGAAGC
CTGGGAAAAGTACGACGAACCATATAAAATTACGTATCCGGAGTACGTATCGATCCAACGGGAG
AAAGATTCTGGAGCTTATAGCATTAAGGCCGCGTTAGAGCGTGATGGATTCGTGGACCGTGCCG
ATCCTGGGTGGGTTTCCACTATGCAACTTCACTTTGGAGCTATAGCCCTCGAAGAATATGCAGC
TTCAACTGCCGAGGCAAGGATGGCCAGATTCGCAAAAGCGCCTGGTAATCGAAACATGGCCACA
TTCGGAATGATGGATGAGAACCGACACGGACAAATTCAGCTTTATTTTCCGTATGCTAACGTTA
AAAGAAGTAGAAAGTGGGATTGGGCACATAAAGCTATTCACACTAATGAATGGGCCGCTATAGC
CGCTAGGAGCTTCTTTGATGATATGATGATGACGAGAGACAGTGTAGCTGTCTCGATCATGCTT
ACTTTCGCATTCGAGACAGGGTTCACGAATATGCAATTCCTTGGCCTTGCAGCGGATGCGGCGG
AAGCAGGAGATCACACATTTGCATCTCTAATTTCGTCCATCCAAACAGATGAATCGAGACATGC
GCAGCAAGGTGGACCAAGCCTTAAGATACTTGTTGAAAACGGAAAGAAGGATGAAGCACAGCAG
ATGGTCGATGTTGCCATCTGGCGTTCCTGGAAACTATTTAGCGTTTTAACAGGACCTATTATGG
ACTACTACACACCTCTTGAGAGTCGAAATCAGTCTTTCAAGGAATTTATGTTAGAATGGATTGT
TGCTCAATTTGAACGTCAATTGCTCGATCTTGGACTTGACAAGCCCTGGTATTGGGATCAATTT
ATGCAAGATCTTGACGAAACTCATCACGGAATGCACCTTGGCGTTTGGTACTGGCGGCCAACGG
TTTGGTGGGACCCAGCGGCGGGAGTTTCTCCTGAGGAGAGGGAGTGGCTTGAAGAAAAGTACCC
AGGTTGGAATGACACCTGGGGACAGTGCTGGGATGTCATCACGGATAATCTCGTTAATGGCAAG
CCTGAGCTAACCGTACCGGAGACATTACCAACCATTTGCAATATGTGCAACTTACCAATCGCTC
ACACTCCAGGAAATAAATGGAATGTCAAGGATTACCAGCTAGAGTACGAAGGCAGATTGTACCA
CTTTGGGAGCGAGGCCGACCGTTGGTGTTTCCAGATCGACCCTGAGCGGTACGAAAACCATACT
AACCTGGTGGACCGATTCTTGAAGGGTGAAATTCAACCGGCAGACCTCGCGGGTGCCCTGATGT
ACATGAGCCTTGAACCAGGAGTTATGGGAGATGATGCGCACGACTATGAATGGGTCAAAGCCTA
TCAGAAGAAAACAAATGCTGCTTGA
-Exemplary Pseudomonas stutzeri Toluene, O-xylene monooxygenase
oxygenase subunit alpha (TouA-P-sp-OX) Amino Acid Sequence
SEQ ID NO: 248
MSMLKREDWYDLTRTTNWTPKYVTENELFPEEMSGARGISMEAWEKYDEPYKITYPEYVSIQRE
KDSGAYSIKAALERDGFVDRADPGWVSTMQLHFGAWALEEYAASTAEARMARFAKAPGNRNMAT
FGMMDENRHGQIQLYFPYANVKRSRKWDWAHKAIHTNEWAAIAARSFFDDMMMTRDSVAVSIML
TFAFETGFTNMQFLGLAADAAEAGDHTFASLISSIQTDESRHAQQGGPSLKILVENGKKDEAQQ
MVDVAIWRSWKLFSVLTGPIMDYYTPLESRNQSFKEFMLEWIVAQFERQLLDLGLDKPWYWDQF
MQDLDETHHGMHLGVWYWRPTVWWDPAAGVSPEEREWLEEKYPGWNDTWGQCWDVITDNLVNGK
PELTVPETLPTICNMCNLPIAHTPGNKWNVKDYQLEYEGRLYHFGSEADRWCFQIDPERYKNHT
NLVDRFLKGEIQPADLAGALMYMSLEPGVMGDDAHDYEWVKAYQKKTNAA
-Exemplary Pseudomonas aeruginosa benzene monooxygenase oxygenase
subunit (BmoA-Pa) Nucleic Acid Coding Sequence
SEQ ID NO: 249
ATGGCTGTATTGAATCGGACGGACTGGTACGACGTCGCCAGAACAACTAATTGGACGCCGAAAT
ATGTCACGGAGGACGAGCTGTTTCCGCCGGAGCTGAGCGGCAGCTTCGATATCCCCATGGAGAA
ATGGGAGGCCTATGACGAGCCCTACAAGCAGACCTATCCCGAATACGTCAAGGTGCAGCGGGAA
AAGGATGCGGGTGTCTACTCGGTCAAGGCGGCCCTCGAGCGCAGCAAGATGTTCGAGAACGCCG
ATCCGGGCTGGCAATCGGTATTGAAATTGCACTTCGGAGCCATCCCCAGCGGCGAATATGCCGC
GTCCACCGCCGAGGCGCGGATGATGCGCTTCTCCAAGGCACCGGGTATGCGCAACATGGCGACG
CTGGGTAGCATGGATGAAATTCGGCACGCGCAACTGCAGCTCTATTTTCCGCACGAGCATGTCT
CGAAGGACCGTCAGTTCGACTGGGCGCACAAGGCATTCGACACCAACGAATGGGCCGCGATCGC
GTCACGCCACTTCTTCGACGACATCATGATGGCGCGCGATGCCATCAGTGTCGGCATCATGCTC
ACCTTCGGGTTCGAGACCGGTTTCACCAACATGCAGTTCCTCGGGCTGGCGGCGGACGCCGCCG
AGGCGGGGGACTTCACCTTCTCCAGCCTGATCTCCAGCATCCAGACCGACGAATCGCGCCACGC
TCAGATCGGCGGGCCTACGCTGCAGATCCTGATCGAAAACGGCAGGAAGGAAGAGGCCCAGAAG
AAGGTGGACATCGCGTTCTGGCGCGCGTGGAGGCTGTTCTCGGTACTGACCGGCCCGATCATGG
ACTACTACACGCCGCTGGAGCACCGCAATCAGTCGTTCAAGGAATTCATGCAGGAGTGGATCGT
CGAGCAGTTCGAGCGTTCCATTCACGATCTGGGGCTGGACAAGCCCTGGTATTGGGACATCTTC
CTGGAGCAACTGGACCAGCAACATCACGGCATGCATCTGGGCGTCTGGTACTGGCGACCCACCG
TCTGGTGGAACCCGACAGCCGGCGTTACGCCCGAAGAGCGCGACTGGCTCGAAGAAAAATACCC
GGGTTGGAACGACACCTGGGGCCACTGTTGGGACGTGATCATCGACAACCTGGTGGAAGGCCGG
ACCGAACTCACCCTGCCGGAAACCCTGCCGATCGTATGCAACATGTGCAACCTCCCGATCAACT
ACACGCCAGGCAACGGCTGGAATGTCCAGGATTATTCGCTCGAATACAACGGACGCCTGTATCA
CTTCGGCTCGGAGCCGGATCGCTGGATCTTCGAGCAGGAACCCGAACGCTATGCGGGTCACATG
ACCCTGGTGGACCGCTTCCTGGCCGGATTGATCCAGCCAATGGACCTGGGTGGCGCCCTGGCCT
ATATGGACCTCGCGCCGGGCGAGAGCGGTGACGATGCACATGGCTATTCCTGGGTCGAGGTCTA
CAAGCAGTTGCGCACGAAAAAAGCGAGTTGA
-Exemplary Pseudomonas aeruginosa benzene monooxygenase oxygenase
subunit (BmoA-Pa) Amino Acid Sequence
SEQ ID NO: 250
MAVLNRTDWYDVARTTNWTPKYVTEDELFPPELSGSFDIPMEKWEAYDEPYKQTYPEYVKVQRE
KDAGVYSVKAALERSKMFENADPGWQSVLKLHFGAIPSGEYAASTAEARMMRFSKAPGMRNMAT
LGSMDEIRHAQLQLYFPHEHVSKDRQFDWAHKAFDTNEWAAIASRHFFDDIMMARDAISVGIML
TFGFETGFTNMQFLGLAADAAEAGDFTFSSLISSIQTDESRHAQIGGPTLQILIENGRKEEAQK
KVDIAFWRAWRLFSVLTGPIMDYYTPLEHRNQSFKEFMQEWIVEQFERSIHDLGLDKPWYWDIF
LEQLDQQHHGMHLGVWYWRPTVWWNPTAGVTPEERDWLEEKYPGWNDTWGHCWDVIIDNLVEGR
TELTLPETLPIVCNMCNLPINYTPGNGWNVQDYSLEYNGRLYHFGSEPDRWIFEQEPERYAGHM
TLVDRFLAGLIQPMDLGGALAYMDLAPGESGDDAHGYSWVEVYKQLRTKKAS
-Exemplary Pseudomonas mendocina Toluene-4-monooxygenase system,
ferredoxin--NAD(+) reductase component (TmoF-Pm) Nucleic Acid Coding
Sequence
SEQ ID NO: 251
ATGTTCAATATTCAATCGGATGATCTCCTGCACCATTTTGAGGCGGATAGTAATGACACTCTAC
TTAGTGCTGCTCTACGTGCTGAATTGGTATTTCCATATGAGTGTAACTCAGGAGGGTGCGGCGC
ATGTAAGATCGAGCTGCTTGAGGGAGAGGTCTCTAACCTATGGCCTGATGCACCAGGATTAGCC
GCCCGTGAACTCCGTAAGAATCGTTTTTTGGCGTGCCAGTGCAAACCATTATCCGACCTCAAAA
TTAAGGTCATTAACCGTGCGGAGGGACGTGCTTCACATCCCCCCAAACGTTTCTCGACTCGAGT
AGTTAGTAAGCGCTTCCTCTCTGACGAGATGTTTGAGCTGCGACTTGAAGCGGAACAGAAAGTG
GTGTTTTCACCAGGGCAATATTTTATGGTTGACGTGCCTGAACTCGGCACCAGAGCATACTCCG
CGGCAAACCCTGTTGATGGAAACACACTAACGCTGATCGTAAAAGCAGTGCCGAATGGGAAGGT
ATCCTGCGCACTCGCAAATGAAACTATTGAAACACTTCAGTTGGATGGTCCTTACGGGCTGTCA
GTATTAAAAACTGCGGATGAAACTCAATCCGTCTTTATCGCTGGGGGGTCAGGTATCGCGCCGA
TGGTGTCGATGGTGAATACGCTGATTGCCCAAGGGTATGAAAAACCGATTACGGTGTTTTACGG
TTCACGGCTAGAAGCTGAACTGGAAGCGGCCGAAACCCTGTTTGGGTGGAAAGAAAATTTAAAA
CTGATTAATGTGTCGTCGAGCGTGGTGGGTAACTCGGAGAAAAAGTATCCGACCGGTTATGTCC
ATGAGATAATTCCTGAATACATGGAGGGGCTGCTAGGTGCCGAGTTCTATCTGTGCGGCCCGCC
GCAGATGATTAACTCCGTCCAGAAGTTGCTTATGATTGAAAATAAAGTACCGTTCGAAGCGATT
CATTTTGATAGGTTCTTTTAA
-Exemplary Pseudomonas mendocina Toluene-4-monooxygenase system,
ferredoxin--NAD(+) reductase component (TmoF-Pm) Amino Acid Sequence
SEQ ID NO: 252
MFNIQSDDLLHHFEADSNDTLLSAALRAELVFPYECNSGGCGACKIELLEGEVSNLWPDAPGLA
ARELRKNRFLACQCKPLSDLKIKVINRAEGRASHPPKRFSTRVVSKRFLSDEMFELRLEAEQKV
VFSPGQYFMVDVPELGTRAYSAANPVDGNTLTLIVKAVPNGKVSCALANETIETLQLDGPYGLS
VLKTADETQSVFIAGGSGIAPMVSMVNTLIAQGYEKPITVFYGSRLEAELEAAETLFGWKENLK
LINVSSSVVGNSEKKYPTGYVHEIIPEYMEGLLGAEFYLCGPPQMINSVQKLLMIENKVPFEAI
HFDRFF
-Exemplary Methylibium petroleiphilum Toluene monooxygenase alpha
subunit (TbuA1-Mp) Nucleic Acid Coding Sequence
SEQ ID NO: 253
ATGGCCCTTCTTGAGAGAATGGATTGGTATGATCTAGCCCGAACCACCAATTGGACACCGACTT
ATGTCTCCGAGGCGGAATTGTTTCCGACCGAAATGTCTGGGGATATGGGAATACCTATGTCTGA
ATGGGAGAAATATGATGAGCCCTACAAGCAGACCTATTCAGAATACGTCAAAATCCAGCGTGAG
AAAGACAGCGGTGCCTACTCTGTGAAGGGTGCCCTTGAAAGAAGCAAAATGTTGGAAAACGCTG
ACCCTGGCTGGATCTCCGTTATCAAAGCACACTATGGAGCAATCGCCAGGGCTGAATACGCGGC
AGCTTCTGCTGAGTCTCGTATGGCCAGGTTCGCCAAAGCACCAGGGCAACGTAACATGGCAACA
ATGGGTATGTTAGACGAGATCAGACATGGCCAGATCCAATTGTTCTTCCCACATGAGCATGTAT
CAAAAGACAGACAATTTGACTGGGCTTTTAAAGCCTACGACACGAATGAGTGGGGAGCAATCGC
TGCTCGTCATATGTTTGATGACATGATGAACACACGTAGCGCTGTGGCTATCGGCCTCATGTTA
ACATTCGCATTCGAGACTGGCTTCACGAACATGCAATTTCTGGGACTGGCAGCAGATGCAGCTG
AAGCAGGTGACTGGACGTTTGCTAGTATGATCTCAAGTGTACAGACTGACGAGTCACGACATGC
TCAGATAGGTGGACCCCTCGTGCCAATCCTGATCGCTAACGGAAAGAAGGCAGAGGCACAGCGT
ATGATTGACGTAGCCTTTTGGCGTAGCTGGAAATTGTTCACAGTTTTAACGGGTCCGATGATGG
ACTATTACACACCTCTCGCTCATCGTAAGCAGTCATTTAAGGAATTTATGCAAGAATTTATCGT
AACTCAATTCGAGCGATCTATATTGGATCTTGGGTTGGAAAGACCCTGGTACTGGGATCAATTC
CTTGCAGAACTAGACTATCAGCACCACGGGATGCACTTAGGTGTGTGGTTTTGGCGTCCTACAG
TTTGGTGGAATCCTGCGGCAGGAGTCACGCCTGAAGAGAGAGCATGGTTAGAAGAAAAGTACCC
AGGTTGGAACGATACTTGGGGCAAATCATGGGACGTTATTGTGGATAATTTATTAAAAGACAAA
CGAGAGCTGACCTATCCGGAGACATTGCCGGTAGTCTGTAATATGTGCAACCTTCCCATCAATG
CTACACCTGGGGACCCTTGGAAAGTTCGTGACCACTCCCTGGAGAGGAAATCGAGATGGTACCA
CTTCTGTTCCGAAGGCTGTAAGTGGTGCTTCGAGCAAGAGCCTGAAAGATACGAGGGCCACCTT
TCTCTTATCGACAGGTTTCTTGCAGGGTTGATCCAGCCAATGGACCTAGGAGGAGGACTCAAAT
ATATGGGATTAGCGCCTGGAGAGATAGGTGACGACGCTCACGGATATGCCTGGTTGGACGCATA
TAGGCAGGTGCCAAAGGCAGCAGCATAA
-Exemplary Methylibium petroleiphilum Toluene monooxygenase alpha
subunit (TbuA1-Mp) Amino Acid Sequence
SEQ ID NO: 254
MALLERMDWYDLARTTNWTPTYVSEAELFPTEMSGDMGIPMSEWEKYDEPYKQTYSEYVKIQRE
KDSGAYSVKGALERSKMLENADPGWISVIKAHYGAIARAEYAAASAESRMARFAKAPGQRNMAT
MGMLDEIRHGQIQLFFPHEHVSKDRQFDWAFKAYDTNEWGAIAARHMFDDMMNTRSAVAIGLML
TFAFETGFTNMQFLGLAADAAEAGDWTFASMISSVQTDESRHAQIGGPLVPILIANGKKAEAQR
MIDVAFWRSWKLFTVLTGPMMDYYTPLAHRKQSFKEFMQEFIVTQFERSILDLGLERPWYWDQF
LAELDYQHHGMHLGVWFWRPTVWWNPAAGVTPEERAWLEEKYPGWNDTWGKSWDVIVDNLLKDK
RELTYPETLPVVCNMCNLPINATPGDPWKVRDHSLERKSRWYHFCSEGCKWCFEQEPERYEGHL
SLIDRFLAGLIQPMDLGGGLKYMGLAPGEIGDDAHGYAWLDAYRQVPKAAA
-Exemplary Pseudomonas putida aromatic ring-hydroxylating dioxygenase
subunit alpha (todC1(bnzA)-Pp) Nucleic Acid Coding Sequence
SEQ ID NO: 255
ATGAACCAAACTGACACCTCACCCATCCGACTACGACGGTCGTGGAATACCAGTGAGATTGAGG
CATTGTTTGATGAGCACGCCGGTAGGATTGATCCTAGAATTTATACGGATGAGGACCTTTATCA
GCTTGAGCTTGAGAGAGTCTTTGCTAGGTCATGGTTGCTCTTGGGGCATGAAACCCAAATTCGG
AAACCAGGTGACTACATTACAACCTACATGGGGGAGGACCCAGTGGTTGTGGTTAGACAAAAAG
ATGCGAGTATAGCGGTATTTTTAAACCAATGCAGGCATAGAGGGATGAGAATTTGTAGAGCCGA
TGCAGGCAACGCTAAGGCTTTTACATGCAGTTATCATGGGGGGCATACGATACCGCAGGCAAC
TTGGTCAATGTACCTTATGAGGCGGAAAGCTTTGCTTGCTTGAATAAAAAGGAGTGGTCCCCCT
TAAAAGCCCGCGTGGAAACCTACAAGGGACTGATATTTGCCAATTGGGATGAAAACGCCGTTGA
CCTCGATACCTATTTGGGTGAAGCAAAGTTTTATATGGACCATATGTTGGATCGGACAGAAGCA
GGGACTGAAGCAATTCCCGGGGTACAAAAATGGGTGATTCCCTGTAATTGGAAATTTGCCGCAG
AACAATTTTGTTCTGATATGTATCACGCTGGCACCACTTCACATCTCAGTGGGATCCTTGCTGG
CCTTCCAGAGGACTTAGAGATGGCTGACTTGGCACCACCGACTGTTGGGAAACAATATCGCGCA
TCATGGGGTGGCCACGGTAGTGGTTTTTATGTTGGAGATCCCAATTTGATGCTGGCCATAATGG
GTCCAAAAGTTACATCATATTGGACTGAAGGGCCCGCCTCCGAGAAGGCCGCTGAGCGGTTAGG
TTCGGTAGAGCGTGGGTCCAAATTGATGGTAGAACACATGACTGTTTTCCCCACCTGTAGTTTT
CTGCCCGGAATAAATACAGTGAGGACTTGGCATCCTCGGGGACCAAACGAGGTGGAAGTATGGG
CGTTTACTGTGGTAGATGCGGACGCTCCGGACGATATAAAAGAAGAGTTTCGTAGACAAACCCT
CAGAACTTTCTCTGCTGGCGGTGTATTTGAGCAAGATGACGGGGAAAATTGGGTGGAGATTCAA
CACATTCTTCGGGGTCACAAGGCTCGCTCTCGTCCCTTTAACGCAGAGATGAGCATGGATCAAA
CTGTGGATAATGATCCTGTTTATCCAGGGCGAATTTCTAATAACGTGTACAGTGAGGAAGCGGC
ACGAGGATTATACGCTCATTGGCTTAGGATGATGACTTCTCCGGACTGGGATGCTTTGAAAGCT
ACTAGGTGA
-Exemplary Pseudomonas putida aromatic ring-hydroxylating
dioxygenase subunit alpha (todC1(bnzA)-Pp) Amino Acid Sequence
SEQ ID NO: 256
MNQTDTSPIRLRRSWNTSEIEALFDEHAGRIDPRIYTDEDLYQLELERVFARSWLLLGHETQIR
KPGDYITTYMGEDPVVVVRQKDASIAVFLNQCRHRGMRICRADAGNAKAFTCSYHGWAYDTAGN
LVNVPYEAESFACLNKKEWSPLKARVETYKGLIFANWDENAVDLDTYLGEAKFYMDHMLDRTEA
GTEAIPGVQKWVIPCNWKFAAEQFCSDMYHAGTTSHLSGILAGLPEDLEMADLAPPTVGKQYRA
SWGGHGSGFYVGDPNLMLAIMGPKVTSYWTEGPASEKAAERLGSVERGSKLMVEHMTVFPTCSF
LPGINTVRTWHPRGPNEVEVWAFTVVDADAPDDIKEEFRRQTLRTFSAGGVFEQDDGENWVEIQ
HILRGHKARSRPFNAEMSMDQTVDNDPVYPGRISNNVYSEEAARGLYAHWLRMMTSPDWDALKA
TR
-Exemplary Pseudoxanthomonas sp. BD-a59 hydroxylase alpha subunit
(tmoA-P-sp-Bda59) Nucleic Acid Coding Sequence
SEQ ID NO: 257
ATGCAATTCCTAGGCCTAGCTGCTGACGCCGCCGAAGCAGGAGATCACACATTTGCTTCATTGA
TCAGCTCAATACAGACTGACGAATCTAGGCATGCTCAGATCGGTGGACCAGCCTTACAGGTTCT
TATTGCTAACGGCCAAAAGGCCACGGCTCAGAAGAAGGTTGATATTGCATTTTGGAGAGCATGG
AAACTATTTGCCGTGTTAACGGGACCAATGATGGACTACTATACTCCACTTGAACACCGAAAAC
AGAGTTTCAAGGAGTTTATGGAAGAGTGGATCGTAGCTCAGTTCGAACGTGCTTTGACTGATTT
AGGTCTTGATTTGCCCTGGTATTGGGACCACTTCCTAGAAGAACTTAGCCAGACACACCACGGA
ATGCACCTGGGAGTATGGTTTTGGCGTCCAACTGTCTGGTGGAACCCAGCCGCTGGGGTAACAC
CAACGGAAAGAGATTAA
-Exemplary Pseudoxanthomonas sp. BD-a59 hydroxylase alpha subunit
(tmoA-P-sp-BDa59) Amino Acid Sequence
SEQ ID NO: 258
MQFLGLAADAAEAGDHTFASLISSIQTDESRHAQIGGPALQVLIANGQKATAQKKVDIAFWRAW
KLFAVLTGPMMDYYTPLEHRKQSFKEFMEEWIVAQFERALTDLGLDLPWYWDHFLEELSQTHHG
MHLGVWFWRPTVWWNPAAGVTPTERD
-Exemplary Pseudomonas mendocina hydroxylase alpha subunit (tmoA-Pm)
Nucleic Acid Coding Sequence
SEQ ID NO: 259
ATGGCAATGACCCTCGGAAAGACTGGTACGAATTGACCAGAGCTACAAATTGGACGCCTTCATA
CGTTACTGAGGAACAGCTTTTCCCCGAGAGAATGTCCGGGCACATGGGAATACCACTTGAGAAA
TGGGAATCCTACGACGAACCATATAAGACATCATATCCAGAGTATGTCTCTATTCAGCGAGAGA
AGGACGCTGGCGCTTACTCTGTTAAGGCGGCGCTCGAACGTGCTAAGATCTATGAAAACTCTGA
CCCTGGCTGGATAAGCACATTGAAGTCACACTACGGAGCAATAGCGGTTGGCGAATACGCGGCT
GTAACTGGTGAGGGACGAATGGCTCGGTTTTCGAAAGCCCCTGGGAATCGTAACATGGCTACTT
TTGGGATGATGGATGAGCTGAGGCACGGACAGTTACAACTGTTCTTTCCACATGAGTATTGCAA
GAAGGACAGACAATTCGATTGGGCATGGAGAGCATATCATAGCAATGAATGGGCCGCCATAGCT
GCTAAACACTTCTTCGACGACATCATCACCGGCAGGGACGCAATCTCAGTCGCGATCATGTTAA
CATTCTCATTCGAGACGGGTTTTACTAACATGCAGTTCCTAGGATTGGCCGCAGACGCAGCAGA
AGCAGGCGATTATACGTTTGCCAATCTTATATCTTCTATCCAGACCGATGAATCCAGACACGCA
CAGCAAGGTGGCCCGGCCCTTCAATTGCTCATAGAAAACGGAAAACGAGAAGAGGCGCAGAAGA
AGGTCGATATGGCTATCTGGAGAGCATGGAGACTTTTCGCAGTCCTGACAGGACCTGTTATGGA
CTACTATACACCATTAGAAGATAGATCTCAATCATTCAAAGAATTTATGTACGAATGGATTATT
GGGCAGTTCGAGCGTTCTCTAATAGACCTTGGTTTGGATAAACCATGGTACTGGGACCTTTTCC
TAAAAGATATTGACGAATTACACCACTCTTATCACATGGGTGTGTGGTATTGGCGAACGACAGC
ATGGTGGAACCCTGCTGCTGGAGTTACTCCCGAGGAGAGAGACTGGCTTGAAGAGAAGTATCCA
GGATGGAACAAGAGATGGGGACGTTGTTGGGACGTAATTACCGAAAATGTATTGAATGACCGGA
TGGATTTGGTCAGCCCGGAAACTTTGCCGTCAGTGTGCAATATGTCCCAGATCCCTCTGGTTGG
TGTCCCGGGCGATGACTGGAACATTGAGGTTTTCAGCCTAGAGCACAACGGAAGGTTGTACCAC
TTTGGGTCCGAAGTGGACAGATGGGTTTTCCAACAGGACCCGGTTCAATACCAAAACCACATGA
ACATCGTAGATCGGTTTCTCGCCGGACAGATCCAACCTATGACGCTTGAAGGGGCACTTAAGTA
CATGGGTTTTCAATCCATTGAGGAGATGGGCAAAGACGCACACGACTTCGCATGGGCCGACAAA
TGCAAACCTGCTATGAAGAAGAGCGCCTAG
-Exemplary Pseudomonas mendocina hydroxylase alpha subunit (tmoA-Pm)
Amino Acid Sequence
SEQ ID NO: 260
MAMHPRKDWYELTRATNWTPSYVTEEQLFPERMSGHMGIPLEKWESYDEPYKTSYPEYVSIQRE
KDAGAYSVKAALERAKIYENSDPGWISTLKSHYGAIAVGEYAAVTGEGRMARFSKAPGNRNMAT
FGMMDELRHGQLQLFFPHEYCKKDRQFDWAWRAYHSNEWAAIAAKHFFDDIITGRDAISVAIML
TFSFETGFTNMQFLGLAADAAEAGDYTFANLISSIQTDESRHAQQGGPALQLLIENGKREEAQK
KVDMAIWRAWRLFAVLTGPVMDYYTPLEDRSQSFKEFMYEWIIGQFERSLIDLGLDKPWYWDLF
LKDIDELHHSYHMGVWYWRITAWWNPAAGVTPEERDWLEEKYPGWNKRWGRCWDVITENVLNDR
MDLVSPETLPSVCNMSQIPLVGVPGDDWNIEVESLEHNGRLYHFGSEVDRWVFQQDPVQYQNHM
NIVDRFLAGQIQPMTLEGALKYMGFQSIEEMGKDAHDFAWADKCKPAMKKSA
-Exemplary Pinus taeda Eng-Phenylalanine Hydroxylase (PHOH-Pt)
Nucleic Acid Coding Sequence
SEQ ID NO: 261
ATGGCGTTTCCACTCCAGAAAACTTTTCTCTGCTCAAATGGCCAATCATTCCCCTGCTCAAATG
GCCGATCGACATCTACACTGCTAGCATCCGACCTCAAGTTTCAACGACTTAATAAGCCTTTCAT
CCTCAGAGTCGGAAGCATGCAAATCAGAAATAGTCCTAAAGAACACCCAAGAGTGAGCAGCGCA
GCTGTGTTGCCTCCAGTACCAAGATCTATTCACGACATACCTAATGGTGATCATATTCTTGGGT
TTGGGGCAAATTTAGCAGAAGATCATCCAGGATACCATGATGAAGAATACAAGAGAAGGCGGTC
ATGTATTGCTGACCTGGCCAAGAAACACAAAATAGGAGAACCCATTCCTGAGATCAACTATACT
ACTGAAGAAGCTCATGTTTGGGCAGAAGTCCTTACAAAGCTTAGTGAATTGTACCCCAGTCATG
CTTGCAAAGAGTATTTGGAATCATTTCCACTTTTCAACTTTTCTCCTAACAAAATTCCTCAACT
AGAAGAGCTTTCACAGATTTTGCAGCATTACACTGGTTGGAAAATAAGACCTGTTGCAGGGCTG
TTGCACCCACGTCAATTTTTGAATGGACTAGCTTTCAAAACATTCCATTCAACACAGTATATTC
GTCACACTAGCAATCCAATGTACACTCCTGAACCTGACATTTGCCATGAGATACTTGGTCACAT
GCCAATGCTTGTACACCCTGAGTTTGCTGATCTTGCTCAGGTTATTGGCTTAGCATCACTGGGA
GCATCAGATAAAGAAATTTGGCATCTTACTAAGCTATATTGGTATACAGTTGAGTTTGGAACAA
TTGAAGAAAATAAGGAAGTTAAGGCATTTGGAGCTGGCATACTGTCAAGTTTTGGTGAGCTTCA
ACACATGAAGTCTAGCAAACCAACATTTCAGAAACTTGATCCATTCGCTCAGCTACCCAAGATG
AGTTACAAGGATGGATTTCAAAATATGTACTTCTTATGTCAAAGTTTTTCAGACACTACAGAAA
AGCTTCGCTCCTATGCAAGAACTATTCACTCTGGTAATTAA
-Exemplary Pinus taeda Eng-Phenylalanine Hydroxylase (PHOH-Pt)
Amino Acid Sequence
SEQ ID NO: 262
MAFPLQKTFLCSNGQSFPCSNGRSTSTLLASDLKFQRLNKPFILRVGSMQIRNSPKEHPRVSSA
AVLPPVPRSIHDIPNGDHILGFGANLAEDHPGYHDEEYKRRRSCIADLAKKHKIGEPIPEINYT
TEEAHVWAEVLTKLSELYPSHACKEYLESFPLFNFSPNKIPQLEELSQILQHYTGWKIRPVAGL
LHPRQFLNGLAFKTFHSTQYIRHTSNPMYTPEPDICHEILGHMPMLVHPEFADLAQVIGLASLG
ASDKEIWHLTKLYWYTVEFGTIEENKEVKAFGAGILSSFGELQHMKSSKPTFQKLDPFAQLPKM
SYKDGFQNMYFLCQSFSDTTEKLRSYARTIHSGN

Phenol and/or Phenol (like) Metabolizing Enzymes

In certain embodiments, a composition described herein comprises at least one transgenic phenol and/or phenol (like) metabolizing enzyme. In certain embodiments, exemplary phenol and/or phenol (like) metabolizing proteins utilize substrates such as phenol and/or phenol (like) to produce intermediate metabolic products such as catechol and/or catechol (like).

In some embodiments, a phenol and/or phenol (like) metabolizing enzyme gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 264, 266, or 268 (or a portion thereof). In some embodiments, a phenol and/or phenol (like) metabolizing enzyme gene and/or transgene comprises a nucleotide sequence that is at least 90%, 91%, 92%, 93%, 94%, 95%, 96%, 97%, 98%, 99%, or 100% identical to any one of SEQ ID NOs: 263, 265, or 267 (or a portion thereof).

-Exemplary Pseudomonas sp. OX1 phenol hydroxylase component phP
(PH-PS-OX1) Nucleic Acid Coding Sequence
SEQ ID NO: 263
ATGAGTTACACCGTCACTATTGAGCCGATCGGCGAGCAGATTGAGGTAGAGGATGGCCAGACTA
TCCTCGCCGCCGCCCTGCGCCAGGGTGTCTGGCTGCCCTTTGCCTGCGGCCACGGCACCTGTGC
TACCTGTAAGGTTCAGGTGCTTGAAGGTGATGTCGAGATCGGAAACGCCTCGCCCTTTGCGCTG
ATGGATATCGAACGTGACGAGGGCAAGGTTCTGGCCTGCTGCGCCACGGTTGAGAGCGACGTCA
CCATTGAGGTGGACATCGATGTGGATCCGGATTTTGAGGGCTACCCGGTGGAGGACTATGCCGC
CATAGCGACCGATATCGTCGAACTCTCTCCGACCATCAAGGGCATTCACCTGAAACTGGACCGG
CCGATGACATTCCAGGCCGGCCAGTACATCAATATCGAACTGCCGGGTGTTGAAGGCGCGAGGG
CCTTCTCCCTGGCCAACCCGCCCAGCAAAGCAGACGAAGTGGAGCTGCATGTGCGCCTCGTTGA
GGGCGGTGCTGCCACCACCTACATCCACGAACAACTGAAAACGGGTGATGCGCTGAACCTTTCA
GGCCCTTACGGCCAGTTCTTCGTGCGTAGTTCCCAACCCGGCGATCTGATTTTCATCGCCGGCG
GATCCGGATTGTCCAGTCCCCAGTCGATGATCCTTGATCTGCTTGAGCAGAACGATGAGCGCAA
GATCGTTCTGTTCCAGGGTGCCCGAAACCTGGCAGAGCTTTACAACCGGGAGCTGTTTGAGGCT
CTGGATCGCGACCACGACAATTTCACCTACGTACCGGCGCTTAGCCAAGCCGACGAAGACCCTG
ACTGGAAGGGCTTCCGAGGCTATGTCCATGAGGCGGCCAACGCCCATTTCGATGGCCGGTTTGC
CGGTAACAAGGCATACCTGTGCGGCCCGCCTCCAATGATCGATGCGGCTATCACGGCATTGATG
CAGGGGCGGCTGTTCGAGCGTGACATCTTCATGGAGAAATTCCTGACAGCGGCGGACGGAGCTG
AAGACACCCAGCGTTCGGCCCTGTTCAAGAAGATATAG
-Exemplary Pseudomonas sp. OX1 phenol hydroxylase component phP
(PH-PS-OX1) Amino Acid Sequence
SEQ ID NO: 264
MSYTVTIEPIGEQIEVEDGQTILAAALRQGVWLPFACGHGTCATCKVQVLEGDVEIGNASPFAL
MDIERDEGKVLACCATVESDVTIEVDIDVDPDFEGYPVEDYAAIATDIVELSPTIKGIHLKLDR
PMTFQAGQYINIELPGVEGARAFSLANPPSKADEVELHVRLVEGGAATTYIHEQLKTGDALNLS
GPYGQFFVRSSQPGDLIFIAGGSGLSSPQSMILDLLEQNDERKIVLFQGARNLAELYNRELFEA
LDRDHDNFTYVPALSQADEDPDWKGFRGYVHEAANAHFDGRFAGNKAYLCGPPPMIDAAITALM
QGRLFERDIFMEKFLTAADGAEDTQRSALFKKI
-Exemplary Cutaneotrichosporon cutaneum Phenol hydroxylase (PH-CC)
Nucleic Acid Coding Sequence
SEQ ID NO: 265
ATGACCAAGTACAGCGAATCCTACTGCGACGTCCTCATCGTTGGTGCCGGCCCCGCCGGTTTGA
TGGCCGCCCGCGTCCTCTCAGAGTACGTGCGCCAGAAGCCCGACCTCAAGGTCCGCATCATCGA
CAAGCGCTCGACCAAGGTCTACAATGGCCAGGCAGACGGTCTCCAGTGCCGTACCCTCGAGTCT
CTAAAGAACCTTGGTCTTGCCGACAAGATCCTCTCGGAGGCAAACGACATGTCGACGATCGCGC
TCTACAACCCCGACGAGAATGGACACATTCGTCGCACCGACCGCATCCCAGACACCCTCCCCGG
CATCTCGCGCTACCACCAGGTCGTGCTCCACCAAGGCCGGATTGAGAGGCACATCCTCGACTCG
ATTGCGGAGATTTCGGACACCCGTATCAAGGTCGAGCGGCCGCTCATCCCCGAGAAGATGGAGA
TCGACAGCTCCAAGGCTGAGGACCCCGAGGCCTACCCCGTCACGATGACTCTCCGCTACATGAG
TGACCACGAGTCGACTCCTCTACAGTTCGGGCACAAGACCGAGAACAGCCTCTTCCACTCCAAC
CTCCAGACCCAGGAGGAGGAGGATGCCAACTACCGCCTCCCCGAGGGCAAGGAGGCGGGCGAGA
TCGAGACCGTTCACTGCAAGTACGTTATCGGCTGTGACGGTGGCCACTCATGGGTCCGCCGCAC
TCTCGGCTTCGAGATGATTGGCGAGCAGACCGACTACATCTGGGGTGTTCTTGACGCTGTCCCG
GCCTCCAACTTCCCCGACATTCGCTCGCCGTGCGCCATCCACTCTGCCGAGTCTGGCTCGATCA
TGATCATCCCGCGCGAGAACAATCTCGTCCGCTTCTACGTTCAGCTCCAGGCCCGCGCTGAGAA
GGGCGGGCGCGTCGACCGCACCAAGTTTACTCCCGAGGTCGTCATTGCCAACGCAAAGAAAATC
TTCCACCCCTACACCTTTGATGTCCAGCAGCTCGACTGGTTTACTGCCTATCACATTGGCCAGC
GTGTTACTGAGAAGTTCTCGAAGGACGAGCGCGTGTTCATCGCCGGTGACGCTTGCCACACCCA
TTCGCCCAAGGCCGGCCAGGGCATGAACACGTCAATGATGGACACCTACAACCTCGGCTGGAAG
CTCGGTCTCGTACTCACTGGCCGTGCCAAGCGCGACATCCTCAAGACGTACGAGGAGGAGCGCC
ACGCATTCGCACAGGCCCTCATCGACTTTGACCACCAGTTCTCGCGCCTCTTCTCGGGCCGCCC
GGCTAAGGACGTGGCCGATGAGATGGGCGTCTCGATGGACGTGTTCAAGGAGGCATTCGTCAAG
GGCAACGAGTTCGCCTCGGGCACCGCTATCAACTACGACGAGAACCTCGTGACCGACAAGAAGA
GTTCCAAGCAGGAGCTTGCCAAGAACTGCGTTGTCGGAACCCGCTTCAAGTCGCAACCCGTTGT
CCGCCACTCTGAGGGCCTCTGGATGCACTTTGGCGACCGCCTCGTCACCGACGGCCGATTCCGC
ATCATTGTCTTCGCCGGCAAGGCTACCGATGCCACCCAGATGTCCCGCATTAAGAAGTTTTCCG
CCTACCTCGACTCGGAGAACTCGGTCATCTCGCTCTACACCCCCAAGGTCTCTGACCGCAACTC
GCGCATCGACGTCATCACCATTCACTCCTGCCACCGCGATGACATCGAGATGCACGACTTCCCC
GCACCGGCTCTCCACCCCAAGTGGCAATATGACTTCATCTACGCCGACTGCGACTCATGGCACC
ACCCCCACCCCAAGTCCTACCAGGCCTGGGGCGTCGACGAGACCAAGGGTGCCGTCGTGGTCGT
CCGCCCAGACGGCTACACCTCGCTCGTGACCGACCTCGAGGGCACCGCCGAGATTGACCGCTAC
TTCAGCGGTATCCTTGTCGAGCCCAAGGAGAAGTCCGGAGCCCAGACCGAGGCCGACTGGACCA
AGTCAACTGCATAA
-Exemplary Cutaneotrichosporon cutaneum Phenol hydroxylase (PH-CC)
Amino Acid Sequence
SEQ ID NO: 266
MTKYSESYCDVLIVGAGPAGLMAARVLSEYVRQKPDLKVRIIDKRSTKVYNGQADGLQCRTLES
LKNLGLADKILSEANDMSTIALYNPDENGHIRRTDRIPDTLPGISRYHQVVLHQGRIERHILDS
IAEISDTRIKVERPLIPEKMEIDSSKAEDPEAYPVTMTLRYMSDHESTPLQFGHKTENSLFHSN
LQTQEEEDANYRLPEGKEAGEIETVHCKYVIGCDGGHSWVRRTLGFEMIGEQTDYIWGVLDAVP
ASNFPDIRSPCAIHSAESGSIMIIPRENNLVRFYVQLQARAEKGGRVDRTKFTPEVVIANAKKI
FHPYTFDVQQLDWFTAYHIGQRVTEKFSKDERVFIAGDACHTHSPKAGQGMNTSMMDTYNLGWK
LGLVLTGRAKRDILKTYEEERHAFAQALIDFDHQFSRLFSGRPAKDVADEMGVSMDVFKEAFVK
GNEFASGTAINYDENLVTDKKSSKQELAKNCVVGTRFKSQPVVRHSEGLWMHFGDRLVTDGRFR
IIVFAGKATDATQMSRIKKFSAYLDSENSVISLYTPKVSDRNSRIDVITIHSCHRDDIEMHDFP
APALHPKWQYDFIYADCDSWHHPHPKSYQAWGVDETKGAVVVVRPDGYTSLVTDLEGTAEIDRY
FSGILVEPKEKSGAQTEADWTKSTA
-Exemplary Asparagus officinalis uncharacterized protein
A4U43_C04F5180 (PH-AO) Nucleic Acid Coding Sequence
SEQ ID NO: 267
ATGAACACGGGCATTCAGGATGCCCATAATTTAGCCTGGAAAATAAGCTGTTTGTTGAAAGATG
CTGCTTCGCCTTCCCTTATAAAAACTTATGAGTCAGAGCGTAGACCAATTGCCATCTCCAACAC
TGCATTAAGTGTTAATAACTTCAAAGCAGCTATGTCAGTTCCTGCTGCACTTGGTATTGATCCA
ACTGTTGCAAATACAGTTCATCAGGTAATAAACAGTAGTTTTGGATCCATTCTTCCTTCTACTT
TCCAAAAAGCTGCCCTGGAAGGAATTTTTTCCATTGGCCGGGCACAACTCTCGGACTTTGTTCT
GAATGAAAACAATCCACTTGGTTCTTCAAGGCTTGCTAGGCTGAGGGCTATATTTGATGAGGGG
AAGATTGGTTTCAGGTACCTTAAGGGAGCTCTGGTAGCTGACAGTGACAACGAAACACAAGAAA
CGGTAGAAACTGCTGCTACCTATAAGAGAGGGTCAAGGGACTATGTTCCCTCCGGTAAACCTGG
ATCGAGATTGCCACATATGCAACTGAGGATGTTGAATGCATCAGAAAATGAGGATTCTATCTCA
ACCTTGGATCTAATATCTGTAGAAAAACTAGAATTCCTTCTGATTATTGCACCGTTGAAAGACT
CCTACGATGTTGCTCGTGTGGCCTTTAAGGTAGCAGAAACACTCAGAGTCTCACTTAAGGTTTG
TGTGATCTGGGCTCAAGGTTCGGCTCCTGCTGATGCTTCTGGAAGTGGACAGGAAGTGGAGCCC
TGGAAAAATTATGTAGATGTTGAAGAAATTCAGAGGTCAAACTCAAAGTCATGGTGGGAGGTGT
GTCAAATGTCGAACAGGGGGGTCATTTTGGTCAGACCTGATGATCATATTGCATGGAGTACAGA
GATTGATTCTGTTGAGAATATTGTGCAACAAGTGGAAAGAGTCTTCTTCCTAATATTAGGGGCG
GTGAGGACCTCTTCGTAG
-Exemplary Asparagus officinalis uncharacterized protein
A4U43_C04F5180 (PH-AO) Amino Acid Sequence
SEQ ID NO: 268
MNTGIQDAHNLAWKISCLLKDAASPSLIKTYESERRPIAISNTALSVNNFKAAMSVPAALGIDP
TVANTVHQVINSSFGSILPSTFQKAALEGIFSIGRAQLSDFVLNENNPLGSSRLARLRAIFDEG
KIGFRYLKGALVADSDNETQETVETAATYKRGSRDYVPSGKPGSRLPHMQLRMLNASENEDSIS
TLDLISVEKLEFLLIIAPLKDSYDVARVAFKVAETLRVSLKVCVIWAQGSAPADASGSGQEVEP
WKNYVDVEEIQRSNSKSWWEVCQMSNRGVILVRPDDHIAWSTEIDSVENIVQQVERVFFLILGA
VRTSS

Catechol and/or Catechol (Like) Metabolizing Enzymes

In certain embodiments, a composition described herein comprises at least one transgenic catechol and/or catechol (like) metabolizing enzyme. In certain embodiments, exemplary catechol and/or catechol (like) metabolizing proteins utilize substrates such as catechol and/or catechol (like) to produce metabolic products such as 2-hydroxymuconicsemi aldehyde, 2-hydroxymuconicsemi aldehyde (like), and/or cis-Muconate.

In some embodiments, catechol and/or catechol (like) metabolizing enzyme gene and/or transgene comprises a sequence encoding a peptide sequence that is at least 90%, 91%, 92%, 93%, 94%, 95%, 96%, 97%, 98%, 99%, or 100% identical to any one of SEQ ID NOs: 270, 272, 274, 276, 278, 280, or 282 (or a portion thereof). In some embodiments, a catechol and/or catechol (like) metabolizing enzyme gene and/or transgene comprises a nucleotide sequence that is at least 90%, 91%, 92%, 93%, 94%, 95%, 96%, 97%, 98%, 99%, or 100% identical to any one of SEQ ID NOs: 269, 271, 273, 275, 277, 279, or 281 (or a portion thereof).

-Exemplary Pseudomonas sp. JR1 3-isopropylcatechol-2,3-dioxygenase
(Ipbc-P-sp-JR1) Nucleic Acid Coding Sequence
SEQ ID NO: 269
ATGGGCATTAAAAGCTTGGGTTACATGGGGTTCTCTGTAAGTGATGTACCGGCATGGCGCTCGT
TCCTCACCGAAAAAGTGGGTTTGATGGAGGTTGTTGGCTCCGATGAGAATGCCTTATACCGCAT
GGACTCACGCAGTTGGCGGATTGCCGTGGAAAGGGGGGAGGCTGACGACCTAGCATTCGCCGGT
TATGAAGTTGCCAATCCGCTGGCCTTGAAGCTGATTACGGAGCGGCTACGGGAGGCTGGTGTTC
AGGTGAGGACCGGCGACACTGAACTGGCAGAAAAGCGTGGCGTGATGGAACTGGTCTCTTTTGA
AGATCCATTTGGAATGCCGCTGGAAATTTACTACGGGGCTACCGAACTATTCGAGCAGCCTTTC
GTTTCTGGCACTTGTGTCACTGGGTTCCTGACTGGTGACCAAGGAGCTGGGCATTATTTTTATG
CTGTCCCGGATATTGAAGAAGGACTGGCTTTCTATACTGGCATACTGGGTTTCCAGATGTCCGA
CGTCATTGATATAGCTATGGGTCCGGATATTACAGTGCGGGGATACTTTCTTCATTGCAACGGG
CGCCACCACACAATGGCGATCGCGGAGGCTCCGTTACCCAAGAGAGTTCACCATTTTTTGCTGC
AGGCCTTGACGCTGGATGATGTAGGTCATGCGTACGACCGAATCGATGGATTGGGCGACAAATC
TACCGACTCCAATCTTCGGGTGCCGGCAAATAGTGATATTAGGTCCAGCAGGATCACGGCGACG
ATCGGACGCCATGTCAACGATCACATGATTTCCTTTTACGCTGAGACGCCGTCCGGGTTTGAGC
TTGAGTTTGGTTGGGGCGCGCGCGACGTAGATGACCGGTCTTGGGTGATGACGAGGCACAAGCG
CACGGCCATGTGGGGTCATAAATCTATGCGTAATAAGTAA
-Exemplary Pseudomonas sp. JR1 3-isopropylcatechol-2,3-dioxygenase
(Ipbc-P-sp-JR1)Amino Acid Sequence
SEQ ID NO: 270
MGIKSLGYMGFSVSDVPAWRSFLTEKVGLMEVVGSDENALYRMDSRSWRIAVERGEADDLAFAG
YEVANPLALKLITERLREAGVQVRTGDTELAEKRGVMELVSFEDPFGMPLEIYYGATELFEQPF
VSGTCVTGFLTGDQGAGHYFYAVPDIEEGLAFYTGILGFQMSDVIDIAMGPDITVRGYFLHCNG
RHHTMAIAEAPLPKRVHHFLLQALTLDDVGHAYDRIDGLGDKSTDSNLRVPANSDIRSSRITAT
IGRHVNDHMISFYAETPSGFELEFGWGARDVDDRSWVMTRHKRTAMWGHKSMRNK
-Exemplary Pseudomonas putida YLE2_PSEPU Metapyrocatechase
(xylE-Pp) Nucleic Acid Coding Sequence
SEQ ID NO: 271
ATGAAGAAGGGAGTAATGCGACCAGGCCACGTGCAACTACGAGTGCTCAACCTAGAGGCGGCGC
TTACTCACTACAGGGATCTTCTTGGTCTAATCGAAATGGACCGAGACGAACAAGGAAGAGTCTA
TCTCAAGGCTTGGTCGGAAGTGGACAAGTTTTCAGTGGTCCTTCGTGAAGCTGATCAGCCAGGA
ATGGACTTCATGGGTTTTAAGGTCACCGATGATGCCTGTCTTACTCGTTTAGCAGGCGAACTCC
TCGAATTTGGATGCCAGGTTGAAGAGATCCCCGCGGGAGAGTTAAAAGACTGTGGTAGGAGAGT
ACGATTTCTTGCCCCGTCTGGACATTTCTTTGAGCTTTATGCTGAGAAAGAATATACGGGTAAA
TGGGGCATCGAGGAAGTTAACCCTGAAGCATGGCCTAGGGACCTGAAGGGAATGAGAGCGGTGA
GGTTCGACCACTGCTTGATGTACGGAGATGAGCTTCAAGCCACATACGAGCTATTCACAGAAGT
TTTGGGATTTTACTTGGCTGAGCAAGTTATCGAGGATAATGGCACACGAATATCTCAGTTTCTT
TCCTTGAGTACCAAGGCTCACGACGTTGCATTCATACAGCACGCTGAAAAGGGAAAATTCCATC
ACGTTAGTTTCTTTCTCGAAACTTGGGAAGATGTCCTTCGAGCAGCAGACTTGATTTCCATGAC
AGACACTTCAATAGACATAGGCCCGACCAGACATGGCCTAACTCACGGTAAAACGATTTATTTC
TTTGACCCGTCAGGAAACAGAAATGAAGTATTTTGCGGTGGCGACTATAACTATCCTGACCACA
AGCCTGTTACCTGGACAGCGGACCAATTGGGCAAGGCTATTTTCTACCATGATCGTATTTTAAA
TGAAAGATTTATGACAGTCCTGACTTGA
-Exemplary Pseudomonas putida YLE2_PSEPU Metapyrocatechase
(xylE-Pp) Amino Acid Sequence
SEQ ID NO: 272
MKKGVMRPGHVQLRVLNLEAALTHYRDLLGLIEMDRDEQGRVYLKAWSEVDKFSVVLREADQPG
MDFMGFKVTDDACLTRLAGELLEFGCQVEEIPAGELKDCGRRVRFLAPSGHFFELYAEKEYTGK
WGIEEVNPEAWPRDLKGMRAVRFDHCLMYGDELQATYELFTEVLGFYLAEQVIEDNGTRISQFL
SLSTKAHDVAFIQHAEKGKFHHVSFFLETWEDVLRAADLISMTDTSIDIGPTRHGLTHGKTIYF
FDPSGNRNEVFCGGDYNYPDHKPVTWTADQLGKAIFYHDRILNERFMTVLT
-Exemplary Burkholderia sp. DBT1 OX extradiol dioxygenase DbtC
(Dbtc-B-DBT1-OX) Nucleic Acid Coding Sequence
SEQ ID NO: 273
ATGGAAAACATTGGGGTCACAGAATTAGGTTATATCGGAATCGGCGTCAGCGACATGGACGCGT
GGCGGGAATATGCCGCGAACGTCATGGGTCTGGAGGTGCTCGAGGAGGGCGACAAAGATCGATT
CTATTTGCGCCTCGATTATCAGCACCATCGGATCGTGGTTCATAATTCGGGGAGCGATGACTTG
GACTACGCTGGCTGGCGAGTTGCAGGCCCTGAAGAATTTGACCAGATCAAACGCAATCTCGAGA
AAGCCAGAGTCGATTTTCGGCAAGCCGATGCAGCAGAGTGCGACGAGCGTATGGTGTTGGATCT
TGTCAAATTCCTCGATCCGGGCGGTAACCCTACAGAAATCTATCATGGCCCGCGGGTTGACTAT
CACAAACCCTTCCATGCTGGCCGCAGAATGCACGGCCGTTTCTCGACCGGTGATCAAGGGCTCG
GTCATATCGGTCATATCATTCTACGACAGGAAAATCCACAAAAGGCATACGAATTCTACGCAAG
AGTTTTGGGCATGCGTGGATCCGTCGAGTATCACATACCGATTCCACACATCGGAATTACTGCG
AAGCCCATTTTTTTGCATTCCAACGATCGAGACCATTCGGTTGCATTTTTAGGTGGGCCAGCGG
CCAAGCGAATCAATCATTTGATGATCGAAGTCGACAATATCGACGACGTTGGCTATACGCACGA
TATTGTCAGGAAACGGCAGATCCCGGTCGCCGTGCAGCTCGGCAAACATTCGAATGATCAAATG
GTCAGCTTTTATTCGGCAAACCCATCTAATTGGCTGTTCGAATATGGCGCATTAGGACGTAGAG
CGACCTATCAGTCGGAATATTATGTTTCGGACATCTGGGGGCATGAAATTGAAGCAACTGGATA
CGGCCTTGACGTCAAATTGAAAGAATAA
-Exemplary Burkholderia sp. DBT1 OX extradiol dioxygenase DbtC
(Dbtc-B-DBT1-OX) Amino Acid Sequence
SEQ ID NO: 274
MENIGVTELGYIGIGVSDMDAWREYAANVMGLEVLEEGDKDRFYLRLDYQHHRIVVHNSGSDDL
DYAGWRVAGPEEFDQIKRNLEKARVDFRQADAAECDERMVLDLVKFLDPGGNPTEIYHGPRVDY
HKPFHAGRRMHGRESTGDQGLGHIGHIILRQENPQKAYEFYARVLGMRGSVEYHIPIPHIGITA
KPIFLHSNDRDHSVAFLGGPAAKRINHLMIEVDNIDDVGYTHDIVRKRQIPVAVQLGKHSNDQM
VSFYSANPSNWLFEYGALGRRATYQSEYYVSDIWGHEIEATGYGLDVKLKE
-Exemplary Ralstonia pickettii catechol 2,3-dioxygenase (tbuE-RpC)
Nucleic Acid Coding Sequence
SEQ ID NO: 275
ATGGGTGTTCTACGAATCGGCATGCGGCCGGTCGTGGCAGGGAGCTTCGGGCAGCATCACCGTC
TTCAGGCCCCACGCTTCGATCTTGGCCTGCAGCTCGTCGAGGTCGGCATCCTTCTCGACCTTGT
AGGCGAGGTGGTTGAGGCCGGCCTGATCCGACGGCGTGAGGATGAGCGAATACTTGTCCCACTC
GTCCCAGCACTTGAAGTAGACGTTGCCGGCGTTGTCCTGCATCGTCACCTTCATGCCGAGCACG
TTTTCGTAGTGCCGCACGGCGGCGGCCATGTCCATCACCTTCAGGCTGGCATGCTGCAGTTCAA
TCTGCCGAGCGGTCACGAGATGCGGCTCTATGCGATGAAGGAGGTGGTCGGCACCGAGGTGGGC
AGCCGCAACCCCGACCCGTGGCCCGACAACCTCAAGGGCGCTGGCGTGCACTGGCTGGATCATG
CCCTGTTGATGTGCGAGTTGAACCCGGAAGCCGGCGTCAACACGGTTGCCGATAACACGCGCTT
CATGCAGGAGGTGCTGGGCTTCTTCCTGACGGAGCAGGTGGTCGTCGGCCCGGACGGTTGCGTA
CAGGCGGCTGCACGGCTGGCCCGCAGCACCACGCCGCACGACATCGCATTCGTCGGTGGTCCGC
GCAGCGGCCTGCACCACATTGCCTTCTTCCTGGACTCGTGGCACGACGTGCTGAAGGCCGCGGA
TGTCATGGCCAAGAACCAGACGAAGATCGACGTGGCACCCACGCGTCACGGCATCACGCGCGGG
CAGACGATCTACTTCTTCGACCCCAGCGGCAACCGCAACGAGACATTCGCCGGCCTGGGCTACC
TCGCGCAGCCGGATCGTCCCGTCACCACGTGGAGTGAAGACAAGCTGTGGACCGGCATCTTCTA
CCACACCGGCGATACGCTGGTGCCGTCGTTCACCGATGTGTACACCTGA
-Exemplary Ralstonia pickettii catechol 2,3-dioxygenase (tbuE-RpC)
Amino Acid Sequence
SEQ ID NO: 276
MGVLRIGMRPVVAGSFGQHHRLQAPREDLGLQLVEVGILLDLVGEVVEAGLIRRREDERILVPL
VPALEVDVAGVVLHRHLHAEHVFVVPHGGGHVHHLQAGMLQFNLPSGHEMRLYAMKEVVGTEVG
SRNPDPWPDNLKGAGVHWLDHALLMCELNPEAGVNTVADNTRFMQEVLGFFLTEQVVVGPDGCV
QAAARLARSTTPHDIAFVGGPRSGLHHIAFFLDSWHDVLKAADVMAKNQTKIDVAPTRHGITRG
QTIYFFDPSGNRNETFAGLGYLAQPDRPVTTWSEDKLWTGIFYHTGDTLVPSFTDVYT
-Exemplary Pseudomonas putida catechol 1,2-dioxygenase (catA-Pp)
Nucleic Acid Coding Sequence
ATGACCGTGAAAATTTCCCACACTGCCGATGTTCAAGCCTTCTTCAACAAGGTGGCTGGCCTGG
ACCATGCCGAGGGCAACCCACGCTTCAAGCAGATCATCCTGCGCGTCCTGCAGGACACCGCGCG
CCTGGTCGAAGACCTGGAAATCACCGAAGACGAATTCTGGCACGCCATTGACTACCTCAACCGC
CTGGGCGGCCGTAACGAGGCGGGCCTGCTGGCCGCAGGCCTGGGTATCGAGCACTTCCTCGACC
TGCTGCAGGACGCCAAGGACGCCGAAGCCGGCTTGGGTGGCGGCACACCGCGCACCATCGAAGG
CCCGCTGTACGTGGCCGGTGCGCCGCTGGCGCAAGGCGAAGCGCGCATGGATGACGGCACCGAT
CCGGGTGTGGTGATGTTCCTTCAGGGCCAGGTGTTCGATGCCGACGGCAAGCCGCTCGCCGGTG
CCACCGTCGACCTCTGGCACGCCAACACCCAGGGCACTTATTCGTACTTCGATTCGACTCAGTC
CGAATACAACCTGCGCCGCCGCATCATCACCGATGCCGTGGGCCGCTACCGTGCGCGCTCCATC
GTGCCGTCGGGGTACGGCTGCGACCCGCAGGGCACGACCCAGGAATGCCTGGACCTGCTCGGCC
GCCACGGCCAGCGCCCGGCGCACGTGCACTTCTTCATCTCGGCACCTGGGTTCCGCCACCTGAC
CACGCAGATCAACTTGAAGATGCCGCTGCCGCGCGTGATCGCGGTGTTCAGGGCGAGCGCTTTG
CCGAACTGCGAGGGCGACAAGTACCTGTGGGATGACTTCGCCTACGCCACCCGTGACGGGTTGA
TTGGCGAGCTGCGCTTTGTCGCGTTCGACTTCCACCTGCAGGCGGCTGCAGCGCCGGAGGCCGA
AGCGCGCAGCCATCGGCCGCGTGCGTTGCAGGAGGGCTGA
-Exemplary Pseudomonas putida catechol 1,2-dioxygenase (catA-Pp)
Amino Acid Sequence
SEQ ID NO: 278
MTVKISHTADVQAFFNKVAGLDHAEGNPRFKQIILRVLQDTARLVEDLEITEDEFWHAIDYLNR
LGGRNEAGLLAAGLGIEHFLDLLQDAKDAEAGLGGGTPRTIEGPLYVAGAPLAQGEARMDDGTD
PGVVMFLQGQVFDADGKPLAGATVDLWHANTQGTYSYFDSTQSEYNLRRRIITDAVGRYRARSI
VPSGYGCDPQGTTQECLDLLGRHGQRPAHVHFFISAPGFRHLTTQINLKMPLPRVIAVFRASAL
PNCEGDKYLWDDFAYATRDGLIGELRFVAFDFHLQAAAAPEAEARSHRPRALQEG
-Exemplary Pseudomonas reinekei catechol 1,2-dioxygenase (catA-Pr)
Nucleic Acid Coding Sequence
SEQ ID NO: 279
ATGAACGTCAAAATTTCCCACACTGCTGAAGTCCAGAATTTTCTCGAAGAGGCCAGCGGCCTGC
ACAACGACGCCGGCAATCCACGGACCAAGGCGCTGATCTATCGCATCCTGCGTGACTCGGTGAA
CATCATCGAAGACCTCGCCGTGACCCCGGAAGAGTTCTGGAAAGCGGTCAACTACCTGAACGTG
CTGGGTGCGCGTCAGGAAGCCGGACTGGTGGTGGCCGGTCTTGGTCTGGAGCACTACCTCGACC
TGCTGATGGACGCCGAAGACGAGCAGGCCGGCAAATCCGGCGGCACCCCGCGTACCATCGAAGG
CCCGCTGTACGTGGCGGGTGCACCATTGTCCGAAGGCGAAGCGCGCCTGGATGACGGGGTTGAT
CCGGGTGTGACCCTGTTCATGCAAGGCCGCGTGTTCAACACCGCAGGCGAGCCTCTGGCCGGTG
CCGTGGTGGACGTCTGGCACGCCAATACCGGCGGTACCTACTCGTACTTCGACCCGGCCCAATC
GGAATTCAACCTGCGTCGCCGCATCGTCACCGACGCCGATGGCCGCTACCGTTTCCGCAGCATC
GTGCCGTCGGGTTACGGCTGCCCGCCGGACGGTCCGACCCAGCAACTGCTCGATCAACTGGGCC
GTCATGGCCAGCGTCCGGCGCACGTGCACTTCTTCATTTCCGCACCGGATCATCGCCACCTGAC
GACGCAGATCAACCTCGATGGCGAAAAATACCTGCATGACGACTTCGCTTACGCCACCCGTGAC
GAGCTGATCGCCAAGATCACCTTCAGCGACGATCAGCAGCGCGCCGCTGCCTACGGTGTGAGCG
GTCGCTTTGCCGAAATCGAGTTCGATTTCACCCTGCAATCGTCTGCCCAGCCTGAAGAACAACA
GCGCCACGAGCGGGTTCGCGCACTGGAAGACTGA
-Exemplary Pseudomonas reinekei catechol 1,2-dioxygenase (catA-Pr)
Amino Acid Sequence
SEQ ID NO: 280
MNVKISHTAEVQNFLEEASGLHNDAGNPRTKALIYRILRDSVNIIEDLAVTPEEFWKAVNYLNV
LGARQEAGLVVAGLGLEHYLDLLMDAEDEQAGKSGGTPRTIEGPLYVAGAPLSEGEARLDDGVD
PGVTLFMQGRVFNTAGEPLAGAVVDVWHANTGGTYSYFDPAQSEFNLRRRIVTDADGRYRFRSI
VPSGYGCPPDGPTQQLLDQLGRHGQRPAHVHFFISAPDHRHLTTQINLDGEKYLHDDFAYATRD
ELIAKITFSDDQQRAAAYGVSGRFAEIEFDFTLQSSAQPEEQQRHERVRALED
-Exemplary Pseudomonas reinekei catechol 1,2-dioxygenase (salD-Pr)
Nucleic Acid Coding Sequence
SEQ ID NO: 281
ATGACCGTAAAAATCAGCCACACCGCTGAAGTGCAGGACCTGATCAAGGAGGCCGCCGGTTTCA
ACAGCGACCAGGGCAGCCCGCGCCTCAAGCAACTGATGCATCGCCTGATCAGCGACGCCTTCAA
GATCATCGAAGACCTGGAAGTGACCGAAGACGAATTCTGGTTGGCGGTGGATCGCCTGAACAAG
GTCGGCGCCCACGCTGAGTTCGGCTTGCTGCTGCCGGGCCTGAGCATGGAGCACTTCATGGACC
TGCTGCAGGACGCCAAGGACCAGCAGATAGGCCTGGCCGGCGGGACCCCGCGGACCATCGAAGG
GCCTCTGTACGTGGCTAACGCGCCGCTCAGCGAAGGTTTTGCGCGCATGGATGATGGCAGTGAA
GATGACGTCGGCATCCCGCTGTTCATCAAGGGTACGGTCCTCAATACGGACGGCAAGCCGGTGG
CCGGTGCGATCGTTGATCTGTGGCACGCCAACACCAATGGCACCTACTCCTACTTCGACGAGAG
TCAGTCGGCGTTCAACCTGCGTCGCCGGATCAAGACCGACGCTGAAGGCCGTTACACCGCGCGC
AGCATCATTCCGAGCGGTTACGGTGTGAATCCCGAAGGGCCGACCCAGGAATGCCTGAGCGCCC
TGGGCCGCCACGGTCAGCGCCCGGCACATATCCATGTGTTCGTTTCCGCACCGGAACATCGTCA
TCTGACCAGCCAGATCAACCTTGCCGGCGACAAATACCTGTGGGACGACTTCGCCTACGCCACC
CGTGAAGGGCTGGTCGGCGAAGCCAGACTGCTCGACAACGCCGACGCCTCGAAAGCCCATGGTC
TGGACGGGCGACAGTTCGCTGAACTCGAATTCGACTTCGTTCTGCAACCGGCGGTCAACGCCGA
CGATGAACACCGCAGCCAGCGTCCACGCGCCGGCCAATGA
-Exemplary Pseudomonas reinekei catechol 1,2-dioxygenase (salD-Pr)
Amino Acid Sequence
SEQ ID NO: 282
MTVKISHTAEVQDLIKEAAGFNSDQGSPRLKQLMHRLISDAFKIIEDLEVTEDEFWLAVDRINK
VGAHAEFGLLLPGLSMEHFMDLLQDAKDQQIGLAGGTPRTIEGPLYVANAPLSEGFARMDDGSE
DDVGIPLFIKGTVLNTDGKPVAGAIVDLWHANTNGTYSYFDESQSAFNLRRRIKTDAEGRYTAR
SIIPSGYGVNPEGPTQECLSALGRHGQRPAHIHVFVSAPEHRHLTSQINLAGDKYLWDDFAYAT
REGLVGEARLLDNADASKAHGLDGRQFAELEFDFVLQPAVNADDEHRSQRPRAGQ

### Modifying Plant Microbiome Components

Among other things, the present disclosure provides compositions, methods of producing, and methods of using genetically modified plants with optimized microbiomes capable of providing useful catabolic and/or anabolic functions.

In certain embodiments of compositions and methods described herein, relevant microorganisms are screened for certain characteristics prior to their use and/or incorporation into the phytosphere (e.g., phyllosphere, endosphere, and/or rhizosphere). In certain embodiments, microorganisms are able to interact mutualistically with the host plant, are well tolerated by the plant, are tolerated by the plant, and/or are only mildly pathogenic to the plant. In certain embodiments, microorganisms are able to degrade and/or metabolize one or more relevant compounds as described herein (e.g., VOCs, e.g., formaldehyde, methanol, benzene, toluene, ethylbenzene, and/or xylene). In certain embodiments, microorganisms are not known to increase environmental risk and/or have adverse effects on human health.

After uptake in the roots and leaves, plants can metabolize, sequestrate and/or excrete air pollutants. In addition, plant-associated microorganisms play an important role by degrading, detoxifying or sequestrating the pollutants and by promoting plant growth.

In case of air pollution, the surface of leaves and stems is known to adsorb significant amounts of pollutants. Therefore, bacteria living on these surfaces, called the phyllosphere bacteria, might be of high importance.

In certain cases, rainfall causes the flow of pollutants down the aerial tissues and to the soil, where it is absorbed right below the plant. In such embodiments, pollutants can come into contact with the soil, the plant's rhizosphere and the roots

Rhizosphere and/or Container

In certain embodiments, compositions and methods described herein comprise microbes that colonize the rhizosphere, surrounding media (e.g., soil or water), and/or container comprising a host plant. In certain embodiments, these microbes are described as members of the media microbiome. In certain embodiments, such microbes may be growing freely in the media (e.g., soil, water, etc.), and/or in association with the root or other immediate plant surfaces. In certain embodiments, microbes that colonize the rhizosphere of a host plant may also or alternatively colonize the phyllosphere and/or endosphere of a host plant.

In certain embodiments, such microbes may have biodegradation capabilities. In certain embodiments, such microbes may have enhanced biodegradation capabilities.

In certain embodiments, such microbes are not pathogenic or are only mildly pathogenic. In certain embodiments, such microbes interact mutualistically with the host plant, e.g., to promote VOC clearance without significantly reducing host plant endogenous functions (e.g., growth and/or reproduction), preferentially, promoting VOC clearance while improving host plant endogenous functions.

In certain embodiments, microbes that have demonstrated and/or known mutualistic interactions with a plant are prioritized as components of a composition as described herein.

In some embodiments, an exemplary rhizosphere component may be Bacillus metanolcius (PB1) (BmPB1), a bacteria that may be found on the roots or in the nearby soil of certain plants.

In some embodiments, an exemplary rhizosphere component may be Ogataea methanolica (KL1) (OmKL1), a fungal yeast that may be found on the roots or in the nearby soil of certain plants.

In some embodiments, an exemplary rhizosphere component may be Pseudomonas putida (F1) (PpF1), a bacteria that may be found on the roots or in the nearby soil of certain plants.

In some embodiments, an exemplary rhizosphere component may be Phanerochaete chrysosporium (Burdsall) (PcBur), a fungi (basidiomycete) that may be found on the roots or in the nearby soil of certain plants.

In some embodiments, an exemplary rhizosphere component may be Rugosibacter aromaticivorans (Ca6T) (RaCa6), a fungi (basidiomycete) that may be found on the roots or in the nearby soil of certain plants.

In some embodiments, an exemplary rhizosphere component may be a microbe isolated as described herein (e.g., see Example 5).

Phyllosphere and/or Endosphere

In certain embodiments, compositions and methods described herein comprise microbes that colonize the phyllosphere of a host plant. In certain embodiments, microbes that colonize the phyllosphere of a host plant may also or alternatively colonize the rhizosphere and/or endosphere of a host plant.

In certain embodiments, a phyllosphere includes microbes colonizing the leaf (e.g., the upper adaxial surface, and/or the lower abaxial surface) and/or stem surfaces of the plant. In certain embodiments, a majority of phyllosphere dwelling microbes may be bacterial and/or fungal yeasts (e.g., as analyzed by 16S sequencing).

In some cases, leaves have been shown to host several VOC-degrading microorganisms. The phyllosphere is one of the most prevalent microbial habitats on earth: the global bacterial population present in the phyllosphere could comprise up to 10²⁶ cells, fungal populations are generally less numerous, and archaea may be considered a minor component or even not abundant. In some embodiments, phyllosphere communities are affected by a variety of environmental factors, including UV exposure, pollution, nitrogen fertilization, water limitations and high temperature shifts, as well as biotic factors, such as leaf age and the co-presence of other microorganisms. In some embodiments, plant leaves are able to adsorb or absorb air pollutants, and habituated microbes on leaf surface and in leaves (endophytes) are able to biodegrade or transform pollutants into less or nontoxic molecules.

In certain embodiments, microbes that occupy the phyllosphere that have certain biodegradation capabilities are prioritized as preferential components of a composition.

In certain embodiments, microbes that occupy the phyllosphere that are not considered pathogenic are prioritized as preferential components of a composition.

Phyllosphere bacterial communities are generally dominated by Proteobacteria, such as Methylobacterium and Sphingomonas. Beijerinckia, Azotobacter, Klebsiella, and Cyanobacteria like Nostoc, Scytonema, and Stigonema also reside in the phyllosphere (see e.g., Xianying Wei et al., Phylloremediation of Air Pollutants: Exploiting the Potential of Plant Leaves and Leaf-Associated Microbes. Frontiers in Plant Science, 2017).

Dominant fungi in the phyllosphere include Ascomycota, of which the most common genera are Aureobasidium Cladosporium, and Taphrina (Coince et al., 2013; Kembel and Mueller 2014).

Basidiomycetous yeasts belonging to the genera Cryptococcus and Sporobolomyces are also abundant in phyllosphere.

Phylloremediation was first coined by Sandhu et al. (2007), who demonstrated that surface-sterilized leaves took up phenol, and leaves with habited microbes or a inoculated bacterium were able to biodegrade significantly more phenol than leaves alone.

The most efficient species in removal of formaldehyde include Osmunda japonica, Selaginella tamariscina, Davallia mariesii, and Polypodium formosanum. Surprisingly, these efficient plants belong to pteridophytes, commonly known as ferns and fern allies.

Formaldehyde can also be assimilated as a carbon source by bacteria (Vorholt, 2002). Such assimilation occurs in Methylobacterium extorquens through the reactions of the serine cycle (Smejkalova et al., 2010), in Bacillus methanolicus through the RuMP cycle (Kato et al., 2006), and in Pichia pastoris through the xylulose monophosphate cycle (Lüers et al., 1998).

As described herein, in some embodiments, bacteria and fungi used to colonize roots can also colonize leaves and could be used for phylloremediation of formaldehyde, methanol, and/or BTEX in the air.

In some embodiments, an exemplary endosphere component may be Methylobacterium oryzae (CBMB20) (MoCBM), a bacteria that may be found on the leaves of certain plants.

In some embodiments, an exemplary phyllosphere component may be Paraburkholderia phytofirmans (PsJN) (PpPsJ), a bacteria that may be found on the epidermis of certain plants.

In some embodiments, an exemplary phyllosphere component may be Methylobacterium extorquens (PA1) (MePA1), a bacteria that may be found on the leaves of certain plants.

In some embodiments, an exemplary phyllosphere and/or endosphere component may be a microbe isolated as described herein (e.g., see Example 5).

### Compositions

Among other things, the present disclosure provides compositions.

In certain embodiments, a composition comprises a genetically modified plant comprising a modified passive diffusion phenotype. In some embodiments, such a modified passive diffusion phenotype is due to alterations to a plant's stomatal density, trichome density, and/or wax levels.

In certain embodiments, a composition comprises a genetically modified plant comprising a modified VOC metabolism phenotype. In some embodiments, such a VOC metabolism phenotype is due to alterations to a plant's metabolism pathways, particularly pathways that utilize substrates such as but not limited to: formaldehyde, formate, D-xylulose 5-phosphate, benzaldehyde, dihydroxyacetone, D-arabino-3-hexulose 6-phosphate (Hu6P, glycoaldehyde, acetylphosphate, pyruvate, 2-keto-4-hydroxybutyrate (HOBA), 3-hydroxypropionaldehyde (3-HPA), aldehyde, benzene, ethylbenzene, toluene, xylene, phenol, phenol (like), catechol, catechol (like), or any combination of these substrates.

In certain embodiments, a composition comprises a genetically modified plant comprising a modified VOC metabolism phenotype.

In certain embodiments, a composition comprises a genetically modified plant comprising a modified stomatal flux phenotype.

In certain embodiments, a composition comprises a genetically modified plant comprising a modified VOC metabolism phenotype and a modified stomatal flux phenotype.

In certain embodiments, a composition comprises a genetically modified plant comprising a modified VOC metabolism phenotype, and an engineered microbe.

In certain embodiments, a composition comprises a genetically modified plant comprising a modified VOC metabolism phenotype, an engineered microbe, and an active air flow system.

In certain embodiments, a composition comprises a genetically modified plant comprising a modified VOC metabolism phenotype, a modified stomatal flux phenotype, and an active air flow system.

In certain embodiments, a composition comprises a genetically modified plant comprising a modified VOC metabolism phenotype, a modified stomatal flux phenotype, and an engineered microbe.

In certain embodiments, a composition comprises an engineered microbe.

In certain embodiments, a composition comprises an engineered eukaryotic cell.

In certain embodiments, a composition comprises an engineered prokaryotic cell.

In certain embodiments, a composition comprises an engineered microbe comprising a modified VOC metabolism phenotype.

In certain embodiments, a composition comprises an engineered microbe comprising a modified VOC tolerance phenotype.

### Methods

In some embodiments, the present disclosure provides methods of using, making, and/or characterizing compositions described herein.

### Methods of Use

In some embodiments, provided herein are methods of using described compositions for the remediation of indoor air quality.

In some embodiments, provided compositions are utilized to improve the indoor air quality of a single family dwelling.

In some embodiments, provided compositions are utilized to improve the indoor air quality of a multi-family dwelling.

In some embodiments, provided compositions are utilized to improve the indoor air quality of a private building.

In some embodiments, provided compositions are utilized to improve the indoor air quality of a public building.

In some embodiments, provided compositions are utilized to improve the indoor air quality of vehicles.

In some embodiments, provided compositions are utilized to improve the indoor air quality of air-tight compartments (e.g., space shuttles, space stations, decompression chambers, submersibles, etc.,)

In some embodiments, provided compositions are utilized to improve outdoor air quality in areas comprising high levels of pollutants.

### Evaluating Air Quality

In some embodiments, indoor air quality can be assessed prior to, during, and/or after exposure to compositions and methods described herein.

In some embodiments, indoor air quality is assessed for levels of formaldehyde.

In some embodiments, indoor air quality is assessed for levels of methanol.

In some embodiments, indoor air quality is assessed for levels of benzene.

In some embodiments, indoor air quality is assessed for levels of ethylbenzene.

In some embodiments, indoor air quality is assessed for levels of toluene.

In some embodiments, indoor air quality is assessed for levels of xylene.

In some embodiments, indoor air quality is assessed for levels of fine particulate matter.

### Methods of Characterizing

In certain embodiments, compositions are characterized based upon their ability to reduce a level of formaldehyde in an indoor air environment relative to a control composition (e.g., a non-engineered plant and/or microbe).

In certain embodiments, compositions are characterized based upon their ability to reduce a level of methanol in an indoor air environment relative to a control composition (e.g., a non-engineered plant and/or microbe).

In certain embodiments, compositions are characterized based upon their ability to reduce a level of benzene in an indoor air environment relative to a control composition (e.g., a non-engineered plant and/or microbe).

In certain embodiments, compositions are characterized based upon their ability to reduce a level of ethylbenzene in an indoor air environment relative to a control composition (e.g., a non-engineered plant and/or microbe).

In certain embodiments, compositions are characterized based upon their ability to reduce a level of toluene in an indoor air environment relative to a control composition (e.g., a non-engineered plant and/or microbe).

In certain embodiments, compositions are characterized based upon their ability to reduce a level of xylene in an indoor air environment relative to a control composition (e.g., a non-engineered plant and/or microbe).

In certain embodiments, compositions are characterized based upon their ability to impact at least one health outcome of an individual that spends a significant period of time indoors. In such an embodiment, a health outcome of an individual may be compared to a control individual, or may be compared to a control states (e.g., prior to or following exposure to compositions as described herein). Such a health outcome may be but is not limited to: the rate of respiratory illness, cognitive function, and/or well-being.

### Production Methods

### Propagating Plants

In some embodiments, compositions described herein are provided as part of a method of producing a phytoremediating plant, or a method of manipulating, and preferably improving phytoremediating properties of a plant, comprising introducing into a plant cell at least one vector as described herein. In some embodiments, a method entails causing or allowing recombination between a vector and the plant cell genome (e.g., Nuclear, mitochondrial, and/or chloroplastic genetic material) to introduce at least nucleotide sequence encoding a metabolism modifying gene into the plant genome. It may optionally further comprise the steps of regenerating a plant and cultivating it.

In some embodiments, compositions described herein comprise Epipremnum aureum that has been transformed by Agrobacterium tumefaciens comprising a vector of interest. In some embodiments, Epipremnum aureum is transformed through methods known in the art, for example, as described in Kotsuka & Tada “Genetic transformation of golden pothos (Epipremnum aureum) mediated by Agrobacterium tumefaciens”, Plant Cell Tissue Organ Culture, 2008; which is incorporated herein by reference in its entirety.

In some embodiments, compositions described herein comprise Epipremnum aureum that has been propagated through a traditional method such as “eye cutting”. In some embodiments, Epipremnum aureum is propagated through methods known in the art, for example, as described in UC MASTER GARDENERS NAPA COUNTY “Healthy Garden Tips—Plant Propagation” handbook, published in March 2011 by the University of California and found on the internet at “https://ucanr.edu/sites/ucmgnapa/files/81929.pdf”; which is incorporated herein by reference in its entirety.

In some embodiments, following transformation, a plant may be regenerated, e.g. from single cells, callus tissue or leaf discs, as is standard in the art. Most plants can be entirely regenerated from cells, tissues and organs of said plant. Available techniques are known in the art and reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

In some embodiments, compositions described herein comprise Epipremnum aureum that has been regenerated from a callus following transformation. In some embodiments, Epipremnum aureum is regenerated through methods known in the art, for example, as described in Zhang, Chen, and Henny “Direct somatic embryogenesis and plant regeneration from leaf, petiole, and stem explants of Golden Pothos” Plant Cell Reports 2005; which is incorporated herein by reference in its entirety.

In some embodiments, microbes are provided to a plant and/or other media to create a composition suitable for VOC biodegradation.

In some embodiments, microbes are sprayed onto a plant. In some embodiments, plants are dipped into a solution comprising microbes. In some embodiments, microbes are sprayed onto activated charcoal that may act as a microbe and/or VOC absorption depot within a growth media (e.g., soil and/or hydroponic water). In some embodiments, microbes are applied to a suitable microbial growth media. In some embodiments, an interior of a container is coated with a composition comprising microbes. In some embodiments, microbes are supplied as a powder and/or liquid to be added to a plant during regular maintenance (e.g., during watering, fertilizing etc.).

In some embodiments, application of a microbe may occur one time, two times, three times, four times, five times, or greater than five times. In some embodiments, microbes are reapplied every 2 weeks, 4 weeks, 6 weeks, 8 weeks, 10 weeks, or 12 weeks. In some embodiments, microbes are reapplied based upon a method of characterizing as described herein, e.g., when a level of VOC biodegradation no longer meets a known and/or expected level. In some embodiments, microbes are reapplied based upon the measurement of culture forming units found in a sample of a plant microbiome when compared to an appropriate control.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

It is believed that one or ordinary skill in the art can, using the preceding description and following Examples, as well as what is known in the art, to make and utilize technologies of the present disclosure.

### Example 1: Creation, Isolation, and Formulation of Vectors for Plant and/or Microbe Transformation

This example provides information regarding the creation, isolation, and formulation of vectors for plant and/or microbe transformation.

Genetic manipulation techniques were performed using technologies known in the art (e.g. Golden Gate cloning systems) and according to manufacturer's instructions. Genes were cloned from appropriate genomic DNA sources isolated using standard protocols such as miniprep or midiprep. The correct sequence of genes of interest were characterized using PCR followed by restriction enzyme digestion and gel electrophoresis and/or by PCR followed by Sanger Sequencing.

Table 1 comprises promoters utilized herein to isolate, clone, and/or verify certain genes of interest.

TABLE 1
Cloning and Sequencing Primers

| SEQ | | | |
|---|---|---|---|
| ID NO: | Target Gene | Primer Name | Primer Sequence |
| 283 | Formolase | FormolaseqF1 | ATTCCTCTGCCACGGCTATC |
| 284 | Formolase | FormolaseqR1 | TTCTTCCCGCTTCGAGGTCT |
| 285 | Formolase | Formolase_seq_F | GCTGCCTGACGCTATGAGG |
| 286 | Formolase | Formolase_seq_R | GATTCCTTGGAGTCTGCCTAG |
| 287 | FALDHEa | EaFALDH_PT_qF1 | TGGAGGATTTAAGTCTAGGT |
| 288 | FALDHEa | EaFALDH_PT_qR1 | CCCAAAGTCAAATTATGAGT |
| 289 | FALDHEa | Ea_FALDH_R | TCAACCTTCAGCCAATACAC |
| 290 | FALDHEa | Ea_FALDH_F | GTCAATGTCAATGCCAATAA |
| 291 | FALDHEa | FALDH_Ea_seq_F | TGGATTGGGAGCTGTTTGGAATA |
| 292 | FALDHEa | FALDH_Ea_seq_R | TCCTCCATCAGTCAAATCAACCA |
| 293 | FALDH9 | FALDH9_qPCR_F | CTGATGATGGCTATATTGTGG |
| 294 | FALDH9 | FALDH9_qPCR_R | TTACTTCTGTGTTGAGCATT |
| 295 | FALDH9 | FALDH_9_seq_F | CGTATGGATTCAATCTCGGTGGA |
| 296 | FALDH9 | FALDH_9_seq_R | ATCGCCTCTATTTGGTCAGGTAC |
| 297 | GD- | FALDH10_qPCR_F | TTGACTGCGACCTGAACGACCT |
| | FALDH10 | | |
| 298 | GD- | FALDH10_qPCR_R | CGGGACAGAGACTATACCAC |
| | FALDH10 | | |
| 299 | GD- | FALDH_10_seq_F | CATGAAGGTGCCAGAAGGAATG |
| | FALDH10 | | |
| 300 | GD- | FALDH_10_seq_R | GCACCCTGTCCTTTGGTAATTTC |
| | FALDH10 | | |
| 301 | GD- | FALDH11qF1 | CAGAGCATTGCGACATCGG |
| | FALDH11 | | |
| 302 | GD- | FALDH11qR1 | AACATTCACAGCGAGCAC |
| | FALDH11 | | |
| 303 | GD- | FALDH_11_seq_F | GCAAGCAGAGTATTTAAGAGTGCC |
| | FALDH11 | | |
| 304 | GD- | FALDH_11_seq_R | AAAGATCGATTGTCTCAGCACCA |
| | FALDH11 | | |
| 305 | FDH3 | FDH3qF1 | TGGAATCACTTTGCGTCAGG |
| 306 | FDH3 | FDH3qR1 | AGTTTGAGGTTCGCGTCTGG |
| 307 | FDH3 | FDH_3_seq_F | CTTTGCAACACTGAAGGAAGCTA |
| 308 | FDH3 | FDH_3_seq_R | GCCTTTGCTCCATTCTCCAATAT |
| 309 | DASCanbo | DAS_CANBO_q_F1 | GGGAAGCGAACTCGAACAGG |
| 310 | DASCanbo | DAS_CANBO_q_R1 | TTCTTGCTGATTTCGGATGG |
| 311 | DASCanbo | DAS_CANBO_q_F2 | AAGAGGTAAGGTCCCGACTG |
| 312 | DASCanbo | DAS_CANBO_q_R2 | TTTCTTGCTGATTTCGGATG |
| 313 | DASCanbo | DAS_CANBO_q_F3 | GAGGTAAGGTCCCGACTGTG |
| 314 | DASCanbo | DAS_Canbo_seq_F | TGTAATTGGAACGTGATCGAGGT |
| 315 | DASCanbo | DAS_Canbo_seq_R | CTTTTGCAGGAATGTCCGAGAAG |
| 316 | DAKC | DAKCF_q_F1 | CCGCATTAACTTCGCTCTT |
| 317 | DAKC | DAKCF_q_R1 | GCACGTCCCGCATTAGCCT |
| 318 | DAKC | DHAK_Cf_seq_F | TACGCAAAATTCAGCTCAGGTTG |
| 319 | DAKC | DHAK_Cf_seq_R | TCATATCTAATGCGGTAACCAAGC |
| 320 | DAKP | DHAK_Pp_seq_F | TCGATAAGAACGATGAGGTGGTG |
| 321 | DAKP | DHAK_Pp_seq_R | TCTCCTGTCTTTGTAGCGTTCAA |
| 322 | DAKP | DAKpp_F_qPCR | ACGACGGAGCAGAAGCGAC |
| 323 | DAKP | DAKpp_R_qPCR | CGTCAGTGATACCGGAAA |
| 324 | DAKY | DHAK_Sc_seq_F | GATGGTTAACAACATGGGCGG |
| 325 | DAKY | DHAK_Sc_seq_R | TGAGTATATCACCACCAGCCTTG |
| 326 | DAKY | DAK2y_F_qPCR | AGCGGTGGAGAAGCGTTAGA |
| 327 | DAKY | DAK2y_R_qPCR | TGAAGTGCCGCCCATTGAGT |
| 328 | DAKE | DHAK_Ec_seq_F | TTAACTTTGAAACAGCGACCGAG |
| 329 | DAKE | DHAK_Ec_seq_R | CATCGACGGTTTGATCAAGGG |
| 330 | DAKE | DAKec_F_qPCR | AATAATCAAGGCCACTCAA |
| 331 | DAKE | DAKec_R_qPCR | CATGAATGCCGACGCCAAAC |
| 332 | HPS-Bm | HPS_BM_F_qPCR | GGTGGCATCAAGCTAGAAA |
| 333 | HPS-Bm | HPS_BM_R_qPCR | TCCACCACCGACGATAACC |
| 334 | HPS-Mg | HPS_MG_F_qPCR | AAGCAGGTGCCGATTTGGT |
| 335 | HPS-Mg | HPS_MG_R_qPCR | TCCGGCTATAGTTGAGTCGT |
| 336 | HPS/PHI-Bm | HPS/PHI_Bm_Ea_F | GACTTGCAGGCTGTTGGAAAAA |
| 337 | HPS/PHI-Bm | HPS/PHI_Bm_Ea_R | TCATAAGGCCCTGTTTCACAAGT |
| 338 | HPS/PHI-Mg | HPS/PHI_Mg_Ea_F | TACGATCCCTGCTGTCCAAAAAG |
| 339 | HPS/PHI-Mg | HPS/PHI_Mg_Ea_R | GGTCCACCTTGGCTGCTG |
| 340 | HPS/PHI- | HPSPHIaqF1 | ACAACAGGGCGGTAAAGTC |
| | archea | | |
| 341 | HPS/PHI- | HPSPHIaqR1 | TCGCAATATAATCTGTCGG |
| | archea | | |
| 342 | HPS/PHI- | HPS/PHI_a_seq_F | GCCGGTGGATTAAATCTGGAAAC |
| | archea | | |
| 343 | HPS/PHI- | HPS/PHI_a_seq_R | CATTGCATCCACTAGACCTCTCA |
| | archea | | |
| 344 | PHI-Bm | PHI_BM_F_qPCR | ACAATAGCAGCGGTGACAA |
| 345 | PHI-Bm | PHI_BM_R_qPCR | TACCGCGTCATAAAACAA |
| 346 | PHI-Mg | PHI_MG_F_qPCR | GCCGCTTTCACAACCAATCC |
| 347 | PHI-Mg | PHI_MG_R_qPCR | AGCGAACCAGCATACTGAC |
| 348 | TodC1(bnzA)- | TodC1_Ea_F | ATATGTTGGATCGGACAGAAGCA |
| | Pp | | |
| 349 | TodC1(bnzA)- | TodC1_Ea_R | CCAGCATCAAATTGGGATCTCC |
| | Pp | | |
| 350 | TodC1(bnzA)- | Tod-C1_F | GATCTCCCACGTAGAAACCAGATC |
| | Pp | | |
| 351 | TodC1(bnzA)- | Tod-C1_R | GATCTGGATACTTATCTCGGTGAGG |
| | Pp | | |
| 352 | TouA-P-OX | Toua_SP_F | GAGCAACAATCCATTCTAACATAAA |
| | | | TTCC |
| 353 | TouA-P-OX | Toua_SP_R | TCACACATTTGCATCTCTAATTTCG |
| 354 | TbuA1-Mp | TbuA1_F | GGACCCGTTAAAACTGTGAACAATT |
| 355 | TbuA1-Mp | TbuA1_R | TTGATGACATGATGAACACACGTAG |
| 356 | P450-RR | PR450RR_F1 | GTCTCCTATCCGTGTATCAGTTGTT |
| 357 | P450-RR | PR450_R1 | CTTACATTCTATGATGATGGCTGGC |
| 358 | PHOH-Pt | PHE_OH_F | TTTATCGCTCGCACCTAGACTTG |
| 359 | PHOH-Pt | PHE_OH_R | TTCTCCAAACAAGATTCCACAGTTG |
| 360 | BmoA-Pa | Bmoa_AP_F | ATGATCCCCACACTTATAGCATCTC |
| 361 | BmoA-Pa | Bmoa_AP_R | GAAGAAGGTTGATATTGCGTTTTGG |
| 362 | TmoF-Pm | TMOF_PM_F | AAGGTAATCAATCGAGCTGAAGGAA |
| 363 | TmoF-Pm | TMOF_PM_R | TGTCTCAATCGTCTCATTAGCAAGA |
| 364 | Stomagen | AtStomagen_F_qPCR | CAGCACCAACTTGTACG |
| 365 | Stomagen | AtStomagen_R_qPCR | GCACTGTTGATAGGGTC |
| 366 | Stomagen | OsX1/X2_F_qPCR | GTTCGACTGCTCCAATATGC |
| 367 | Stomagen | OsX1/X2_R_qPCR | TACACTTGAATCGACACCCT |
| 368 | Stomagen | NtMyb23_F_qPCR | ATCCGCACAAAGGCAATTAG |
| 369 | Stomagen | NtMyb23_R_qPCR | CAACATGAAAGCGTAAG |
| 370 | Stomagen | AtStomagen_Ea_F | ACTGGGAAACTATGTCGTACAGG |
| 371 | Stomagen | AtStomagen_Ea_R | TCTGCCCTACATTTGTAACGACA |
| 372 | Caprice | AtCaprice_Ea_F | TAATGTTTAGAAGCGACAAGGCC |
| 373 | Caprice | AtCaprice_Ea_R | AAGCCTTTCTGAAAAAGTCTCGC |
| 374 | Caprice | AtCaprice_F_qPCR | GCATAAACGACGACGGAGAC |
| 375 | Caprice | AtCaprice_R_qPCR | CTACTCACCTCTTCGGAACA |
| 376 | Glabra1 | Glabra1_F_qPCR | TGGTGTCCGCGTCCTATG |
| 377 | Glabra1 | Glabra1_R_qPCR | AGTAATGAGACGGGTCGTTG |
| 378 | Glabra2 | Glabra2_F_qPCR | GCCGCTTCTTCCTATCACC |
| 379 | Glabra2 | Glabra2_R_qPCR | CTCATATCCTGACCCGTCTT |
| 380 | Glabra3 | Glabra3_F_qPCR | GGGCTCACTGACAACCTAC |
| 381 | Glabra3 | Glabra3_R_qPCR | CGCACCTCAATTCTATGAC |
| 382 | Chitinase1 | Ea_CHI1_F | GAAGCCGACGAAGAACGACA |
| 383 | Chitinase1 | Ea_CHI1_R | CGGCACAATCCAGATTATCA |
| 384 | Actin | Ea_Act_F | TACAGTGCCCATCTACGAAG |
| 385 | Actin | Ea_Act_R | CCCGTTCAGCCGTTGT |
| 386 | mCherry | mCherry_qpcr_R1 | CTTCAGCTTGGCGGTCTGGG |
| 387 | mCherry | mCherry_qpcr_F2 | CGCCTACAACGTCAACATC |
| 388 | mCherry | mCherry_qpcr_R2 | CGGCGCGTTCGTACTGTTC |
| 389 | TurboGFP | TurboGFP_seq_F | TCTCCATACCTTCTTTCTCACGT |
| 390 | TurboGFP | TurboGFP_seq_R | CTCAACAGTAGCGTTAGACCTGA |
| 391 | HPT | HPT_Ea_F | AACCTGGCGTGACTTTATTTGTG |
| 392 | HPT | HPT_Ea_R | TGACGCCTCTCAAAATACCTTGT |
| 393 | HPT | HPT_seq_F | AAGACCTGCCTGAAACCGAAC |
| 394 | HPT | HPT_seq_R | GGACATTGTTGGAGCCGAAATC |
| 395 | Bar | Bar_seq_F | TCATTACATTGAGACTTCTACTGTGA |
| 396 | Bar | Bar_seq_R | CAATCACAGCAACCACAGACTTG |
| 397 | Kana | KANA_F1 (but reverse | CGGTAAGGATCTGAGCTACACATG |
| | | finally) | |
| 398 | Kana | KANA_F2 (but reverse | CCACAGTCGATGAATCCAGAAAAG |
| | | finally) | |
| 399 | Kana | KANA_R1 (but forward | GCTACCCGTGATATTGCTGAAGAG |
| | | finally) | |
| 400 | Nos | Nos_Pro_R | GAGACTCTAATTGGATACCGAGGG |
| 401 | Nos | Nos_Ter_F | AGCAGATCGTTCAAACATTTGGC |
| 402 | Nos | Nos_terminator_seq_F | GCGCGGTGTCATCTATGTTACTA |

Exemplary constructs as described in Table 2 were created.

TABLE 2
Exemplary Constructs Comprising At Least Two Genes of Interest

| Gene 1 | Gene 2 | Gene 3 | Gene 4 | Gene 5 | Gene 6 | Gene 7 |
|---|---|---|---|---|---|---|
| Bar | FALDH_10 | — | — | — | — | — |
| Bar | FALDH_11 | — | — | — | — | — |
| Bar | HPS/PHI_a | — | — | — | — | — |
| Bar | Formolase | — | — | — | — | — |
| Bar | FALDH_9 | — | — | — | — | — |
| Bar | Formolase | DAK2_Yeast | — | — | — | — |
| Bar | Formolase | DAK_Cf | — | — | — | — |
| Bar | Formolase | DAK_Pp | — | — | — | — |
| Bar | Formolase | DAK_Ec | — | — | — | — |
| Bar | FALDH_11 | FDH_3 (Chloro) | — | — | — | — |
| Bar | FALDH_11 | FDH_3 (Cyto) | — | — | — | — |
| Bar | DAS_Canbo | DAK2_Yeast | — | — | — | — |
| Bar | DAS_Canbo | DAK_Cf | — | — | — | — |
| Bar | DAS_Canbo | DAK_Pp | — | — | — | — |
| Bar | DAS_Canbo | DAK_Ec | — | — | — | — |
| Bar | EaFALDH | FDH_3 (Chloro) | — | — | — | — |
| Bar | EaFALDH | FDH_3 (Cyto) | — | — | — | — |
| Bar | FALDH_9 | FDH_3 (Chloro) | — | — | — | — |
| Bar | FALDH_9 | FDH_3 (Cyto) | — | — | — | — |
| Bar | FALDH_10 | FDH_3 (Cyto) | — | — | — | — |
| Bar | FALDH_10 | FDH_3 (Cyto) | — | — | — | — |
| Bar | EaFALDH | — | — | — | — | — |
| Bar | Dummy | DAK2_Yeast | — | — | — | — |
| Bar | Dummy | DAK_Cf | — | — | — | — |
| Bar | Dummy | DAK_Pp | — | — | — | — |
| Bar | Dummy | DAK_Ec | — | — | — | — |
| Bar | Dummy | FDH_3 (Chloro) | — | — | — | — |
| Bar | Dummy | FDH_3 (Cyto) | — | — | — | — |
| hpt | TurboGFP | — | — | — | — | — |
| Bar | Dummy | FDH3_mito | — | — | — | — |
| Ba | EaFALDH | FDH3_mito | — | — | — | — |
| Bar | FALDH_9 | FDH3_mito | — | — | — | — |
| Bar | FALDH_10 | FDH3_mito | — | — | — | — |
| Bar | FALDH_11 | FDH3_mito | — | — | — | — |
| hpt | FALDH_10 | FDH_3 (Chloro) | — | — | — | — |
| hpt | FALDH_10 | FDH_3 (Cyto) | — | — | — | — |
| hpt | Formolase | DAK2_Yeast | — | — | — | — |
| hpt | Formolase | DAK_Cf | — | — | — | — |
| hpt | Formolase | DAK_Pp | — | — | — | — |
| hpt | Formolase | DAK_Ec | — | — | — | — |
| hpt | DAS_Canbo | DAK2_Yeast | — | — | — | — |
| hpt | DAS_Canbo | DAK_Cf | — | — | — | — |
| hpt | DAS_Canbo | DAK_Pp | — | — | — | — |
| hpt | DAS_Canbo | DAK_Ec | — | — | — | — |
| HPT | ANT1 | — | — | — | — | — |
| HPT | Delila | Rosea1 | — | — | — | — |
| HPT | GhPAP1 | — | — | — | — | — |
| HPT | AtPAP1 | — | — | — | — | — |
| HPT | P35S-eGFP | — | — | — | — | — |
| HPT | CrtW | CrtZ | — | — | — | — |
| HPT | PPvUbi2- | — | — | — | — | — |
| | eGFP | | | | | |
| HPT | PZmUbi1- | — | — | — | — | — |
| | eGFP | | | | | |
| HPT | HispS | H3H | Luz | CPH | — | — |
| HPT | VvMYBA5 | VvMYBA6 | — | — | — | — |
| HPT | ZmPl | ZmLc | — | — | — | — |
| HPT | DAS_Canbo | DHAK-2yeast | — | — | — | — |
| HPT | DAS_Canbo | DHAK-Ec | — | — | — | — |
| HPT | DAS_Canbo | DHAK-cf | — | — | — | — |
| Kana | DAS_Canbo | DHAK-2yeast | — | — | — | — |
| Bar | AtCaprice | — | — | — | — | — |
| Bar | AtStomagen | — | — | — | — | — |
| Bar | OsX1 | — | — | — | — | — |
| Bar | OsX2 | — | — | — | — | — |
| Bar | NtMyb23 | — | — | — | — | — |
| Bar | AtGlabra1 | — | — | — | — | — |
| Bar | FALDH-11 | FDH3_mito | — | — | — | — |
| Kana | DAS_Canbo | Dhak-PP | — | — | — | — |
| Kana | DAS_Canbo | DHAK-cf | — | — | — | — |
| Kana | DAS_Canbo | Dhak-ec | — | — | — | — |
| Bar | FALDH-9 | FDH3_mito | — | — | — | — |
| Bar | DAS_Canbo | DHAK-ec | — | — | — | — |
| BAR | DAS_Canbo | DHAK-cf | — | — | — | — |
| BAR | FALDH_10 | FDH3_mito | — | — | — | — |
| BAR | FALDH-11 | FDH3_cyto | — | — | — | — |
| Kana | TMOF_PM | — | — | — | — | — |
| KANA | TBUA1_Mp | — | — | — | — | — |
| KANA | P450_RR | — | — | — | — | — |
| KANA | Tmoa_SP | — | — | — | — | — |
| KANA | TOD_C1 | — | — | — | — | — |
| KANA | BMOA_PA | — | — | — | — | — |
| KANA | P450_2E1 | — | — | — | — | — |
| KANA | PHE_OH | — | — | — | — | — |
| KANA | Toua-SP | — | — | — | — | — |
| KANA | AtCaprice | — | — | — | — | — |
| KANA | AtStomagen | — | — | — | — | — |
| KANA | OsX1 | — | — | — | — | — |
| KANA | OsX2 | — | — | — | — | — |
| KANA | NtMyb123 | — | — | — | — | — |
| KANA | AtGlabra1 | — | — | — | — | — |
| KANA | AtGlabra2 | — | — | — | — | — |
| KANA | AtGlabra3 | — | — | — | — | — |
| HPT | TMOF_PM | — | — | — | — | — |
| HPT | Tbua1 | — | — | — | — | — |
| HPT | P450_RR | — | — | — | — | — |
| HPT | tmoa_SP | — | — | — | — | — |
| HPT | TOD_C1 | — | — | — | — | — |
| HPT | BMOA_PA | — | — | — | — | — |
| HPT | P450_2E1 | — | — | — | — | — |
| HPT | PHE_OH | — | — | — | — | — |
| HPT | toua_SP | — | — | — | — | — |
| HPT | HPS/PHIA | — | — | — | — | — |
| KANA | HPS/PHIA | — | — | — | — | — |
| BAR | HPS/PHIA | — | — | — | — | — |
| Bar | Formolase | — | — | — | — | — |
| Bar | EaZIP | — | — | — | — | — |
| NptII | HispS | H3H | Luz | CPH | — | — |
| NptII | Delila_mut | Rosea1_mut | — | — | — | — |
| NptII | Delila_mut | Rosea1_mut | — | — | — | — |
| NptII | EaZIP | — | — | — | — | — |
| NptII | Delila_mut | Rosea1_mut | — | — | — | — |
| NptII | Delila_mut | Rosea1_mut | — | — | — | — |
| HPT | AtStomagen | — | — | — | — | — |
| NptII | Delila_mut | Rosea1_mut | — | — | — | — |
| HPT | PvUbi1 + | — | — | — | — | — |
| | 3-eGFP | | | | | |
| HPT | TodC1 (Ea) | EaFALDH- | CrtW (Ea) | CrtZ (Ea) | HPS/PHI_Bm | AtStomagen |
| (Ea) | | IntF2a- | | | (Ea) | (Ea) |
| | | AtFDH1.3 (Ea) | | | | |
| HPT | TodC1 (Ea) | EaFALDH- | CrtW (Ea) | CrtZ (Ea) | — | — |
| (Ea) | | IntF2a- | | | | |
| | | AtFDH1.3 (Ea) | | | | |
| NptII | TodC1 (Ea) | EaFALDH- | CrtW (Ea) | CrtZ (Ea) | HPS/PHI_Bm | AtStomagen |
| | | IntF2a- | | | (Ea) | (Ea) |
| | | AtFDH1.3 (Ea) | | | | |
| NptII | TodC1 (Ea) | EaFALDH- | CrtW (Ea) | CrtZ (Ea) | — | — |
| | | IntF2a- | | | | |
| | | AtFDH1.3 (Ea) | | | | |
| HPT | CaMYBA | CaMYC (Ea) | — | — | — | — |
| | (Ea) | | | | | |
| HPT | FhMYB5 | FhTT8L (Ea) | — | — | — | — |
| | (Ea) | | | | | |

### Example 2: Modification of Epipremnum Aureum

This Example relates to the transformation of Epipremnum Aureum with vectors comprising sequences described herein.

### 1—Agrobacterium-Mediated Transformation

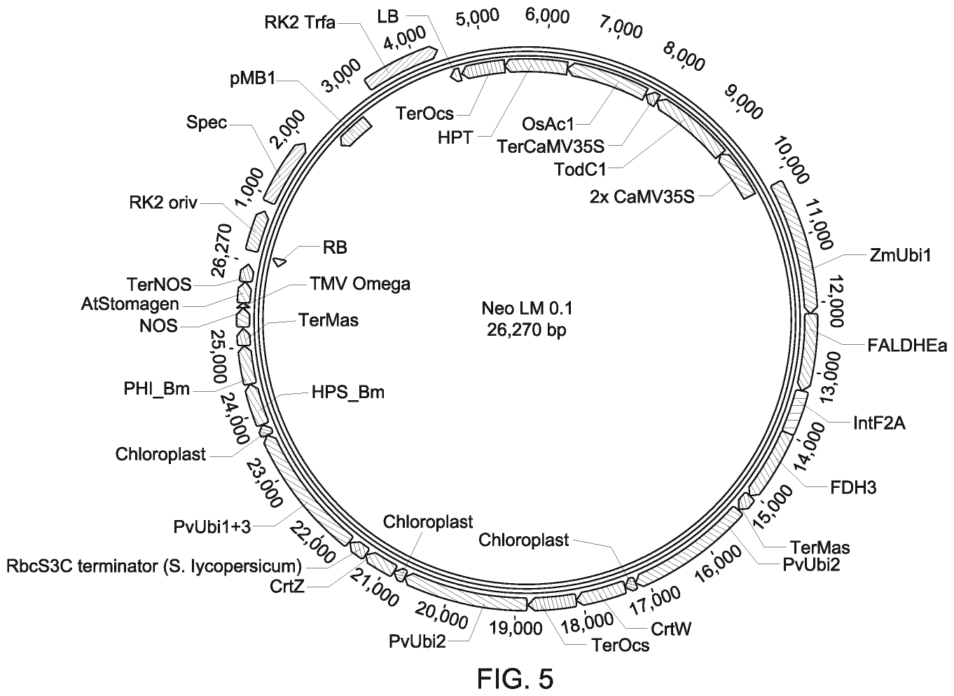

1-1: Preparing material for transformation: young stem and petioles from young pothos were surface-sterilized with a sodium hypochlorite solution (2% chlorine) and a drop of Tween 20 for 25 min with agitation. Explants were then rinsed three times with sterile distilled water and cut into 0.5-1 cm long segments on MS medium (Murashige and Skoog 1962) supplemented with 2.0 mg 1-1N-phenyl-NO—1,2,3-thiadiazol5-yl urea (TDZ), 0.2 mg 1-1 a-naphthalene acetic acid (NAA), 3% sucrose, and 7 gr/L agar and adjusted to pH 5.8 (referred to herein as regeneration media (RM)).
1-2: Agrobacterium preparation for the transformation of golden pothos: A. tumefaciens strain EHA105 containing a plasmid of interest was used for the transformation of golden pothos. The A. tumefaciens strain was grown in 5 ml of LB liquid medium supplemented with 50 mg/L spectinomycin and 30 mg/L rifamycin at 30 C until the absorbance at 600 nm reached 0.8-1.0. The strain was then transformed with a plasmid of interest (for Example, as represented by FIGS. 4 and 5). Plasmids used for transformation comprised a selection marker (e.g., hygromycin phosphotransferase gene driven by the 35S promoter). Following transformation, 25 mg/L hygromycin B was used as a selection agent in the regeneration media.
1-3: Infection and Transformation: pre-cultured pothos stem explants were immersed for 20 minutes in an A. tumefaciens suspension with liquid medium (RM media without agar) supplemented with 0.1 mM acetosyringone, explants were occasional agitated to ensure exposure to A. tumefaciens.
1-4: Co-Incubation: explants were then transferred onto an RM co-incubated media plate and stored for three days in a dark growth chamber at 26° C.
1-5: Selection and embryogenesis: after co-cultivation, explants were rinsed three times with liquid medium, comprising 100 mg/L cefotaxime, 100 mg/L carbenicillin, and 30 mg/L hygromycin. Explants were then returned to a dark growth chamber kept at 26° C. Explants were transferred to fresh medium (RM) every 2-3 weeks to avoid oxidative products released from the hygromycin, these products can induce undesirable necrotic browning tissues. Embryogenic calli were readily observed after approximately 8-12 weeks of culture.
1-6: Shoot generation: hygromycin-resistant embryos were transferred onto germination medium comprising MS-medium supplemented with 0.2 mg 1-1 NAA, 2 mg 1-1 6-benzylaminopurine (BAP), 3% sucrose, and 0.7% Agar (pH 5.8).
1-7: Root generation and transfer to soil: germinated shoots were then transferred onto an MS medium supplemented with 1% sucrose (pH 5.8) in plant boxes for further growth of shoots and roots. Grown plants were transferred to soil to propagate under standard greenhouse conditions with a 16 h/8 h photoperiod at 25°/20° C. day/night, and 60% relative humidity.

### 2—Biolistic Transformation of Pothos

2-1: Preparation of gold particles: for each shot transformation, 1.4-1.5 mg gold particles of 0.6 μm diameter (BioRad, Munich, Germany) were washed with 600 μL pure ethanol, then vortexed for 1 min and shortly centrifuged in a table-top microcentrifuge at 5,000 rpm. Supernatant was removed and particles were washed with 600 μL H2O. Washed gold particles were resuspended in 175 μL H2O and 2 mg of DNA comprising a plasmid of interest (for Example, as represented by FIGS. 4 and 5]), 175 μL CaCl2 (2.5 M stock) and 35 μL spermidine were added, and briefly mixed using a vortex. Suspensions were incubated for 10 minutes on ice and then briefly centrifuged using a table top microcentrifuge. Supernatant was then discarded, and the particle pellet was resuspended in 600 μL ethanol. The mixture was then centrifuged at 5,000 rpm for 1 second after which the supernatant was removed. The particle pellet was resuspended in 60 μL of pure ethanol and dropped (10 μL) on macrocarriers which were placed in the holes of the hepta-adaptor (BioRad). The macrocarriers and hepta-adaptor were sterilized with ethanol before use.
2-2: Biolistic transformation: young leaves and petioles from young pothos plants were sterilized as described in section 1-1 above, and arranged onto the surface of a MS-solid medium comprising 2.0 mg TDZ and 0.2 mg NAA. Prepared explants were then bombarded with plasmid DNA coated onto the gold particles using the DuPont PDS—1000/He biolistic gun.
2-3: Selection and embryogenesis: after transformation leaves were cut into small pieces (˜5×5 mm in size) and placed onto the surface of an MS-based supplement with 25 mg/L Hygromycin.
2-4: Shoot and root generation and transfer to soil: steps as described above in section 1-6 and 1-7 were followed.

In certain cases, a new desirable gene and/or pathway is introduced into a golden pothos plant which is already transformed (e.g., a super-transformation transgenic event). The transformation method is the same as described in section 1 or section 2 of Example 2, except that explants are from pothos that is already transgenic rather than from wild type pothos. In order to select the super-transformation transgenic event, a new selection cassette and selection agent is used.

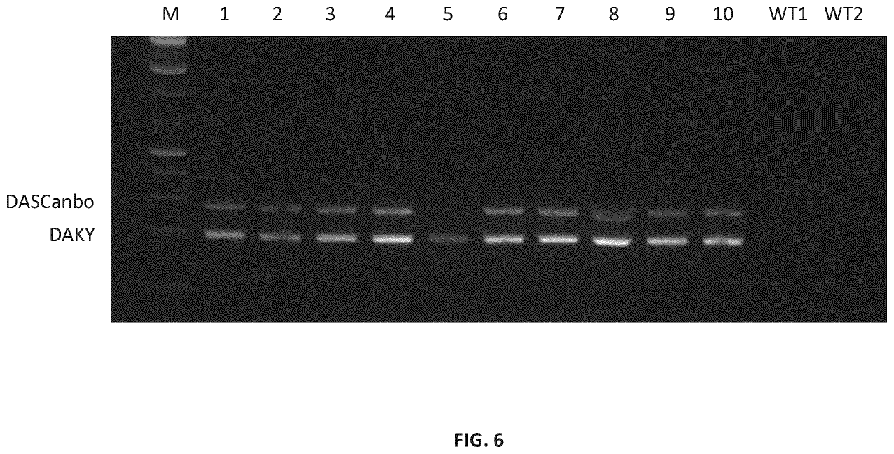

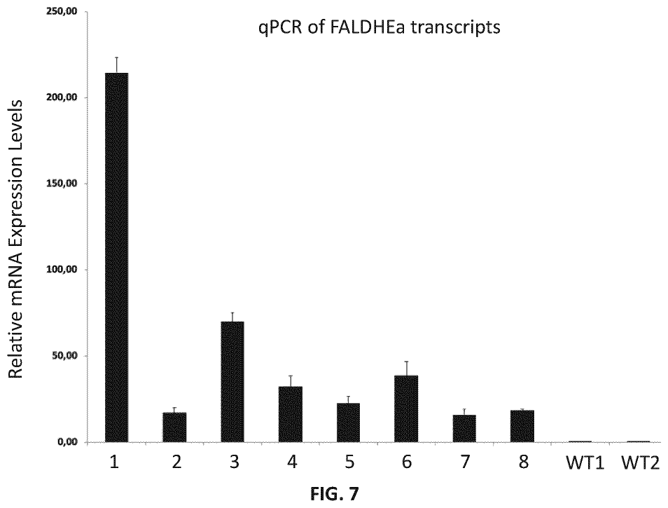

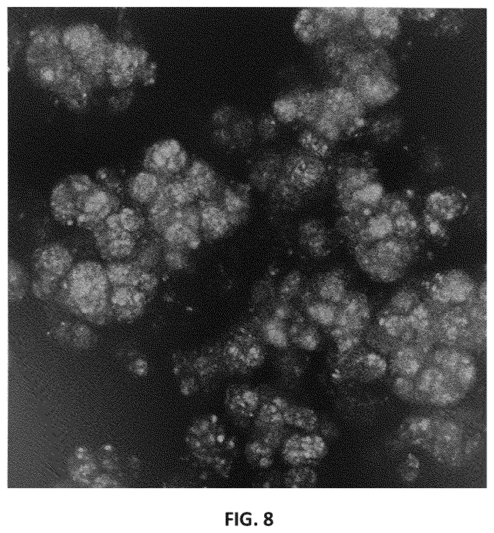

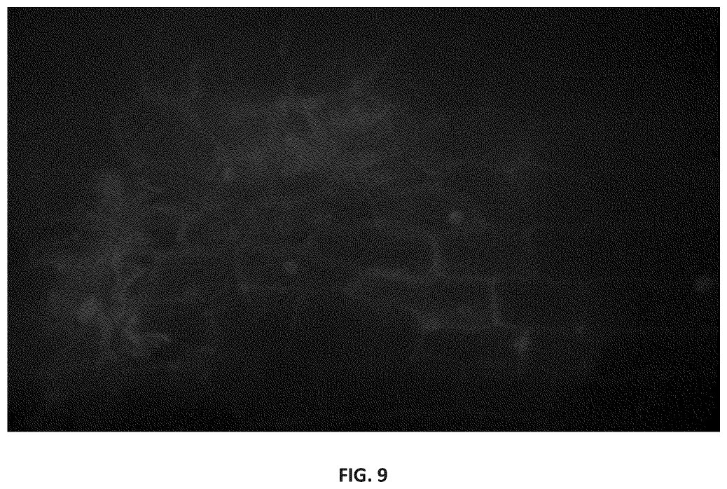

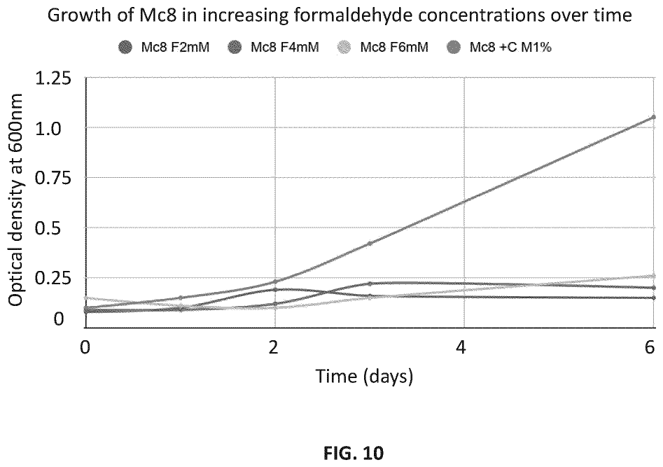

Using a method described herein, a pothos plant was transformed with a composition described herein (see FIG. 4, FIG. 5, FIG. 6, and FIG. 7, FIG. 8, and FIG. 9).

Exemplary constructs found in Table 3 were transformed into golden pothos.

TABLE 3
Exemplary Constructs Transformed Into Golden Pothos

| Gene 1 | Gene 2 | Gene 3 |
|---|---|---|
| hpt | FALDH_10 | FDH_3 (Chloro) |
| hpt | FALDH_10 | FDH_3 (Cyto) |
| hpt | Formolase | DAK2_Yeast |
| Bar | AtCaprice | — |
| Bar | AtStomagen | — |
| Bar | OsX1 | — |
| Bar | OsX2 | — |
| KANA | AtStomagen | — |
| KANA | OsX1 | — |
| KANA | NtMyb123 | — |
| KANA | AtGlabra1 | — |
| KANA | HPS/PHIA | — |
| BAR | HPS/PHIA | — |
| Bar | Formolase | — |

### Example 3: Demonstration of Heterologous Gene Expression in Epipremnum Aureum

This Example relates to the confirmation of heterologous gene expression in transformed Epipremnum aureum.

To confirm transgene introduction into Pothos, approximately 20-30 mg of transformed leaf pieces were collected and placed in a 1.5 mL Eppendorf tube containing 2 stainless steel beads of 3 mm diameter. The tube was then flash frozen in liquid nitrogen and introduced into a mixer mill (Retsch MM400) to lyse the samples (shaking at 30 Hz for 1 minute). Following lysis, 500 μL of GEx buffer was added (5.5 M Guanidine Thiocyanate, 20 nM Tris-HCl, pH 6.6) and the sample was vortexed vigorously. The samples were centrifuged for 5 minutes at 20,000 g and the supernatant was loaded on a Silica Membrane Mini Spin Column (from any DNA purification kit). The column with the sample was centrifuged at 20,000 g for 1 minute and the membranes were washed twice with 750 μL of cleaning buffer (80% ethanol, 10 mM Tris-HCl, pH 7.5). To remove any trace of ethanol, the samples were centrifuged at 20,000×g for 1 min and the genomic DNA was eluted by adding 50 μL of ddH2O to the column followed by centrifugation at 20,000×g for 1 min. The extracted genomic DNA was used in a PCR with primers specific to the transgene of interest (see Table 5) to confirm transgenesis.

PCR was conducted as known in the art. In brief, PCR conditions were as follow: in a 25 μL total reaction volume, 1 μL of DNA, 2.5 μL of 10× FastStart buffer with MgCl2 (Roche), 0.5 μL of 10 mM dNTP (Roche), 2.5 μL of forward primer at 10 mM, 2.5 μL of reverse primer at 10 mM, 0.2 μL of FastStart Taq (Roche, Cat. No. 12 032 937 001) and 15.8 μL of ddH2O. The cycling conditions of the PCR were optimized for each primer pair, but in general were as follows: 95° C. for 4 minutes, 35 cycles of: 95° C. for 30 seconds 55° C. for 30 and seconds 72° C. for 1 minute, 72° C. for 5 minutes, and hold at 12° C. The PCR products were analyzed on a 2.5% agarose gel stained with BET and the fragments size was compared to the known theoretical size using a DNA ladder as reference.

When a pothos plant was confirmed to have integrated a transgene, the transgenes expression level was tested and confirmed by qPCR. In general, qPCR was performed as known in the art, in brief: a leaf sample of 100 mg was taken and placed in a 1.5 mL Eppendorf tube containing 2 stainless steel beads of 3 mm diameter. The tube was then flash frozen in liquid nitrogen and introduced into a mixer mill (Retsch MM400) to lyse the samples (shaking at 30 Hz for 1 minute). RNA extraction was then performed with the Macherey Nagel NucleoSpin RNA Plant, Mini kit for RNA from plant, ref: 740949.50 (according to the manufacturer instructions). Once RNA was purified, qPCR reactions were set up using the NEB Luna® Universal One-Step RT-qPCR Kit (Ref: E3005 L). In a 5 μL total reaction volume, 2.5 μL of Luna Universal One-Step Reaction Mix (2×), 0.5 μL of Luna WarmStart® RT Enzyme Mix (20×), 0.2 μL of forward primer at 10 mM, 0.2 μL of reverse primer at 10 mM, 1 μL of RNA and 0.85 μL of nuclease-free water. Primer efficiency was tested using serial dilutions of the RNA (1 to 10,000 fold), all reactions were performed in at least triplicate. For each RNA sample, a pothos endogenous gene (actin) was used as the reference for calculating expression levels. The reaction was run on a LightCycler® 96 from Roche.

A skilled practitioner of the art will recognize that DNA and RNA extraction protocols, and PCR and qPCR reaction protocols can vary greatly while still producing valuable and informative data.

### Example 4: Air Purification by Transgenic Epipremnum Aureum

This Example relates to indoor air purification by technologies described herein, and the measurement of the same.

Method One (sentinels): A) a magnetic stir bar and stainless steel tripod are placed within a suitable air-tight container (e.g., a sealable glass jar) on top of a stir plate in a controlled environment; B) a product to be tested (e.g., a composition described herein) is placed within the suitable container, placed on top of the tripod in such a way that the stir bar is permitted to spin freely; C) a known and controlled amount of pollutant (e.g., VOC) is introduced into the suitable container; D) a custom built lid that contains at least one sensor for detecting a pollutant are comprised within the suitable container; E) the stir plate is activated to stimulate airflow, sensor outputs are logged every minute and pollutant concentrations over time are determined.

Method two (flow-through system): A) a stable pollutant gas source (e.g., a VOC) is created using a source tank and a permeation tube apparatus; B) a product to be tested is placed inside a suitable air-tight container (e.g., a sealable glass jar); C) the suitable air-tight container is sealed with a custom lid that comprises two pipes passing through it and into the air-tight container, one pipe is an inlet that extends to near the bottom of the jar, and one pipe is an outlet that is flush or near flush with the lid; D) at least one suitable pollutant sensor is calibrated; E) a suitable pollutant sensor measures the output concentration of volatile pollutant, while a suitable pollutant sensor (the same or an additional sensor) measures the input concentration of volatile pollutant; F) the concentration difference between output and input is measured.

Method three (DNPH derivatization cartridges for formaldehyde): A) a magnetic stir bar and stainless steel tripod are placed within a suitable air-tight container (e.g., a sealable glass jar) on top of a stir plate in a controlled environment; B) a product to be tested (e.g., a composition described herein) is placed within the suitable container, placed on top of the tripod in such a way that the stir bar is permitted to spin freely; C) a known and controlled amount of pollutant (e.g., VOC) is introduced into the suitable container; D) the suitable container is sealed using a lid fitted with a septum; E) a suitable period of time is allowed to pass (e.g., 3 hours); F) using a syringe and a needle, 50 ml of the jar contents is aspirated through a derivatization cartridge; F) the derivatization cartridge is extracted and injected into a suitable measurement device (e.g., an HPLC machine) following cartridge manufacturer's instructions.

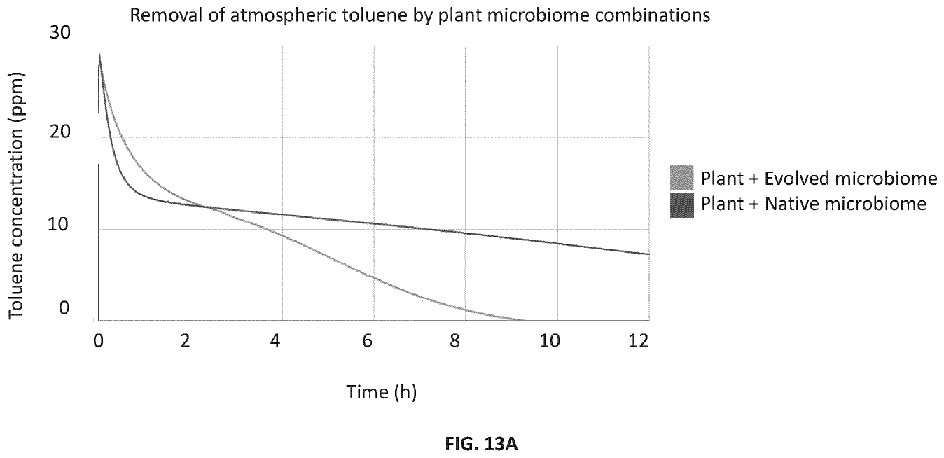

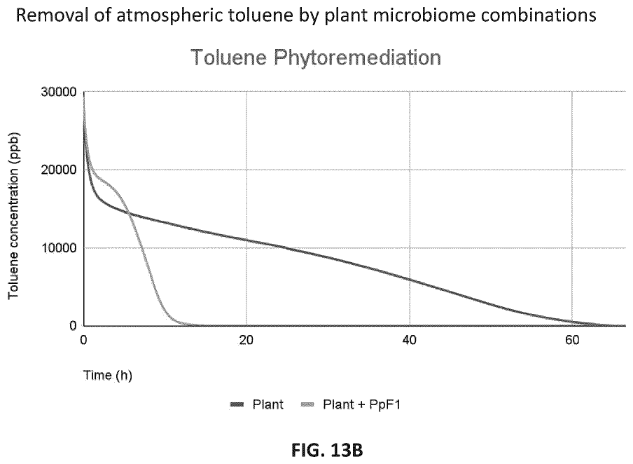

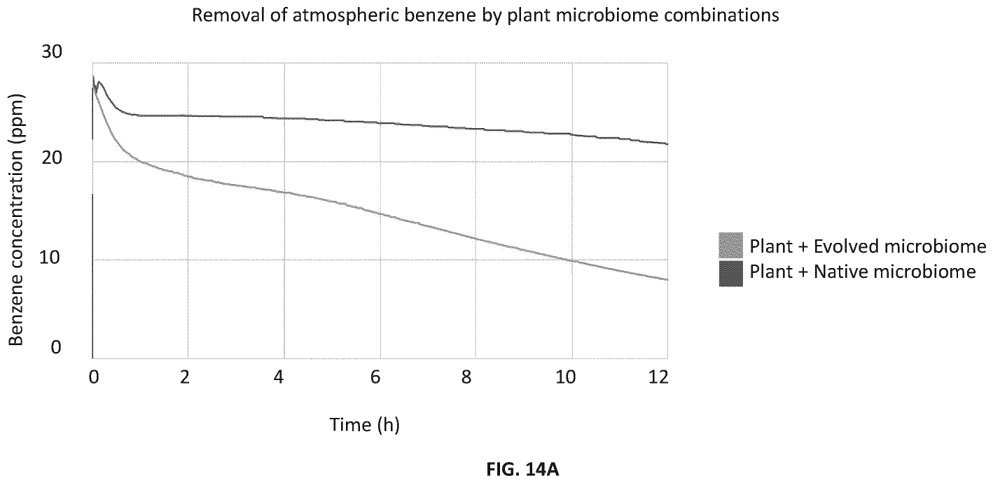

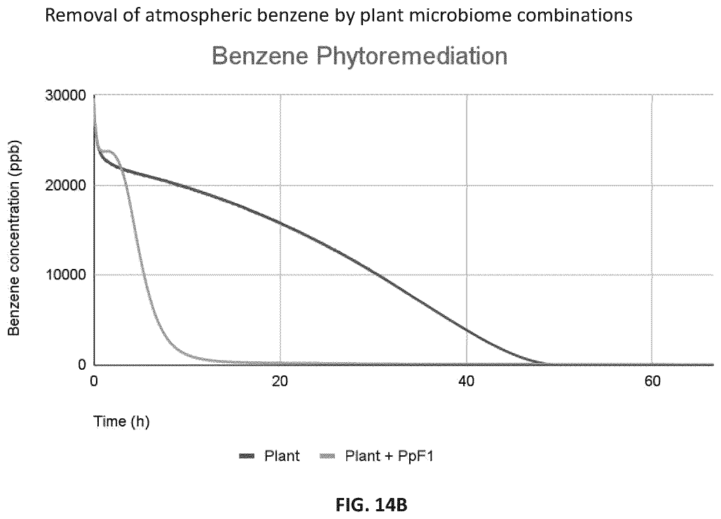

Using methods described herein, a composition comprising a pothos plant and a microbiome was tested for volatile toluene metabolism (see FIG. 13). Using methods described herein, a composition comprising a pothos plant and a microbiome was tested for volatile benzene metabolism (see FIG. 14).

### Example 5: Identification and Characterization of Exemplary Microbiome Components

The current Example relates to discovery of and characterization of microbes suitable for microbiome colonization of certain compositions (e.g., plant tissues, and/or soil/media) described herein. There is little public data on Epipremnum aureum natural microbiome, in some embodiments, methods and compositions described herein are in part a product of detection and characterization of microbes suitable for Epipremnum aureum microbiome colonization. In some embodiments, suitable microbes are identified and isolated from certain plants or from polluted soils.

Host plants are collected from an environment (e.g., any environment, including but not limited to: an endemic region, a green house, or a stress promoting region). Plants aerial regions are conservatively washed to gently remove phyllosphere inhabiting microbes. A phyllosphere suspension is then serial diluted and incubated on various solid media that may be selective or nonselective, permitting growth of phyllosphere microbiome inhabitants of interest. Following or prior to aerial region washing, a host plants soil interfacing regions (e.g., roots) are incubated in an agitated suspension solution to create a soil and rhizosphere microbiome suspension. Such a suspension can be serially diluted, and aliquots are incubated on various solid and/or liquid media that may be selective or nonselective, permitting growth of soil and/or rhizosphere microbiome inhabitants of interest. Following at least a first aerial and/or root washing, host plants undergo a sterilizing wash (e.g., with soap) to remove any additional surface dwelling microbes. Host plants are then dissected, and sections are incubated on various solid media that may be selective or nonselective, permitting growth of endosphere dwelling microbes. Microbes from a phyllosphere, rhizosphere, soil, and/or endosphere are grown to a suitable stage, isolated, banked, and then characterized through genetic, (e.g., by 16S/ITS sequencing) and/or functional means (e.g., pollutant metabolism rates).

Leaves, soil, and roots are collected from a relatively polluted environment (e.g., near a hydrocarbon processing and/or dispensing site). Soil and roots are incubated in an agitated suspension solution to create a soil and rhizosphere microbiome suspension. Such a suspension can be serially diluted, and aliquots are incubated on various solid and/or liquid media that may be selective or nonselective, permitting growth of soil and/or rhizosphere microbiome inhabitants of interest. Leaves are conservatively washed to gently remove phyllosphere inhabiting microbes. A phyllosphere suspension is then serial diluted and incubated on various solid media that may be selective or nonselective, permitting growth of phyllosphere microbiome inhabitants of interest. Microbes from a phyllosphere, rhizosphere, and/or soil are grown to a suitable stage, isolated, banked, and then characterized through genetic, (e.g., by 16S/ITS sequencing) and/or functional means (e.g., pollutant metabolism rates).

Suitable microbes are detected and isolated using a bait technique. Soil is added to an outdoor container (e.g., a pot) in a well ventilated area, pollutants of interest, such as BTEX, formaldehyde, methanol, and/or various hydrocarbons are added to the soil, creating a selective media. The selective media (e.g., soil within a pot) is then enriched with at least one, but preferably as many as feasible, different unique soil samples to increase the microbial diversity found in the selective media. Pollutants of interest are added at regular intervals (e.g., every 12 hours, 24 hours, 48 hours, or 168 hours) during a suitable incubation period (e.g., 1 day, 5 days, 1 week, 2 weeks, 3 weeks, 4 weeks, 2 months, 4 months, 6 months, or 1 year). Following a suitable selection and incubation period, polluted soil is incubated in an agitated suspension solution to create a soil microbiome suspension. Such a suspension can be serially diluted, and aliquots are incubated on various solid and/or liquid media that may be selective or nonselective, permitting growth of soil microbiome inhabitants of interest. Microbes are then grown to a suitable stage, isolated, banked, and then characterized through genetic, (e.g., by 16S/ITS sequencing) and/or functional means (e.g., pollutant metabolism rates).

Suitable microbial consortia are detected and isolated as a population. Polluted soil is collected (e.g., from near a hydrocarbon processing and/or dispensing site), and placed immediately into an agitated solution of minerals and pollutant media. Additional nutrients and pollutants of interest are added at regular intervals (e.g., every 12 hours, 24 hours, 48 hours, or 168 hours) during a suitable incubation period (e.g., 1 day, 5 days, 1 week, 2 weeks, 3 weeks, 4 weeks, 2 months, 4 months, 6 months, or 1 year). Following a suitable selection and incubation period, microbial consortia are banked, and then characterized through genetic, (e.g., by 16S/ITS sequencing) and/or functional means (e.g., pollutant metabolism rates).

Host Epipremnum aureum plants were collected from a greenhouse environment. Plants were conservatively washed to gently remove phyllosphere inhabiting microbes. A phyllosphere suspension was then serial diluted and incubated on various nonselective solid, permitting growth of phyllosphere microbiome inhabitants of interest. Following aerial region washing, a host Epipremnum aureum plants soil interfacing regions (e.g., roots) was incubated in an agitated suspension solution to create a soil and rhizosphere microbiome suspension. Such a suspension was then serially diluted, and aliquots were incubated on various solid and/or liquid media that was either selective or nonselective, permitting growth of soil and/or rhizosphere microbiome inhabitants of interest. Following a first aerial and then root washing, host plants underwent a sterilizing wash (e.g., with soap) to remove any additional surface dwelling microbes. Host plants were then dissected, and sections were incubated on various solid media that was selective or nonselective, permitting growth of endosphere dwelling microbes. Microbes from a phyllosphere, rhizosphere, soil, and/or endosphere were grown to a suitable stage, banked, and then characterized, e.g., by 16S/ITS sequencing. In an exemplary extraction, 43 strains of potential microbiome inhabitants were collected, 21 soil and root epiphytes, 18 endophytes, and 4 leaf epiphytes.

Leaves, soil, and roots were collected from a relatively polluted environment (e.g., near a hydrocarbon dispensing site). Soil and roots were incubated in an agitated suspension solution to create a soil and rhizosphere microbiome suspension. Such a suspension was serially diluted, and aliquots were incubated on various solid and/or liquid media that was either selective or nonselective, permitting growth of soil and/or rhizosphere microbiome inhabitants of interest. Leaves were conservatively washed to gently remove phyllosphere inhabiting microbes. A phyllosphere suspension was then serial diluted and incubated on various solid media that were either selective or nonselective, permitting growth of phyllosphere microbiome inhabitants of interest. Microbes from a phyllosphere, rhizosphere, and/or soil were grown to a suitable stage, banked, and then characterized, e.g., by 16S/ITS sequencing. In an exemplary extraction, 12 strains of potential microbiome inhabitants were collected, 8 soil and root epiphytes, and 4 leaf epiphytes.

### Example 6: Microbe Pollutant Metabolism Characterization

The current Example relates to the characterization of metabolic functions in compositions and methods described herein.

Microbes are tested and characterized using a pollutant (e.g., formaldehyde etc.) as the sole carbon source(s). Said pollutant is dissolved in water, and mineral media (MMB/MP). Various ranges of pollutant are utilized (e.g., 2 mM, 4 mM, 6 mM, 8 mM, 10 mM, or greater than 10 mM), and microbe growth is monitored through regular optical density measurements (e.g., daily measurements of OD600). Concurrently, microbes that act as a positive control can be grown with glucose (MMB), or methanol (MP) media.

Tests are carried out in at least duplicate (e.g., duplicate, triplicate, or more) in glass tubes comprising at least 5 mL of mineral media (MP) with loose caps to facilitate oxygen exchange (formaldehyde stayed in solution). At a suitable time interval (e.g., every 12 hours, every 24 hours, every 48 hours, etc.), an appropriate volume of culture (e.g., 50 μL of culture) is sampled and added to a spectrophotometry plate, where an appropriate volume of perchloric acid (e.g., 50 μL) and an appropriate volume of NASH reagent (e.g., 100 μL) are added. The plate is incubated at an appropriate temperature (e.g., about 60° C.) for a suitable period of time (e.g., about 5 minutes) and immediately read in a spectrophotometer (e.g., a Biotek Epoch2) at an appropriate wavelength (e.g., at 400 nm). The absorbance levels of a control series of known formaldehyde concentrations is done in parallel to allow correlation of absorbance and formaldehyde concentration.

Microbes are tested and characterized using a pollutant (e.g., BTEX, etc.) as a sole carbon source(s). Microbes are streaked, placed, or spotted onto suitable growth media (e.g., minimal media agar plates) and incubated in an air-tight chamber. Various ranges of pollutant (e.g., BTEX, etc.) are added to said chamber either together or alone (e.g., 2 mM, 4 mM, 6 mM, 8 mM, 10 mM, or greater than 10 mM), and microbe growth is qualitatively and/or quantitatively assessed visually at regular intervals during a suitable incubation period. Concurrently, microbes that act as a positive control can be grown with glucose or methanol as the carbon source.

Opportunist methylotrophic microbes were from isolated from plants and/or soil as described in Example 7. Methylotrophic microbes (e.g., “Mc8”) were incubated using formaldehyde as the sole carbon source. Formaldehyde was dissolved in water, and mineral media (MMB/MP) at various concentrations (e.g., 2 mM, 4 mM, 6 mM), with control microbes grown using methanol as the carbon source (e.g., CM1% representing 1% methanol in the media as the sole carbon source).

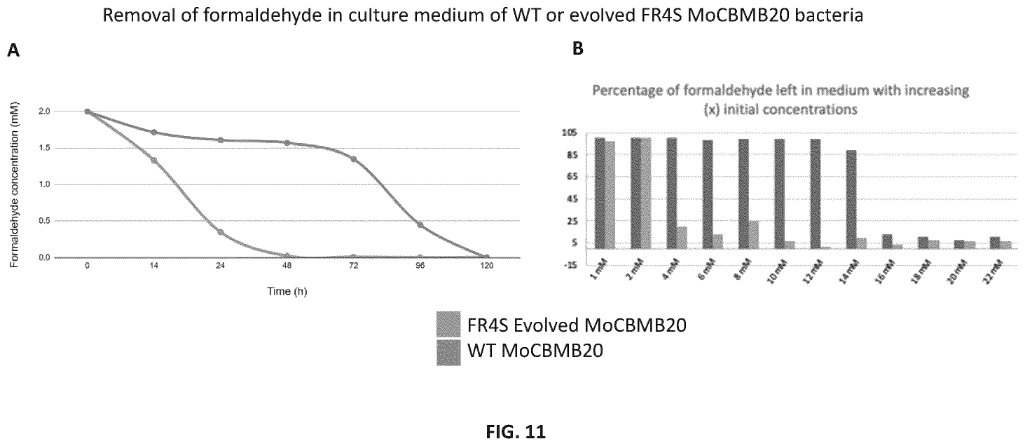

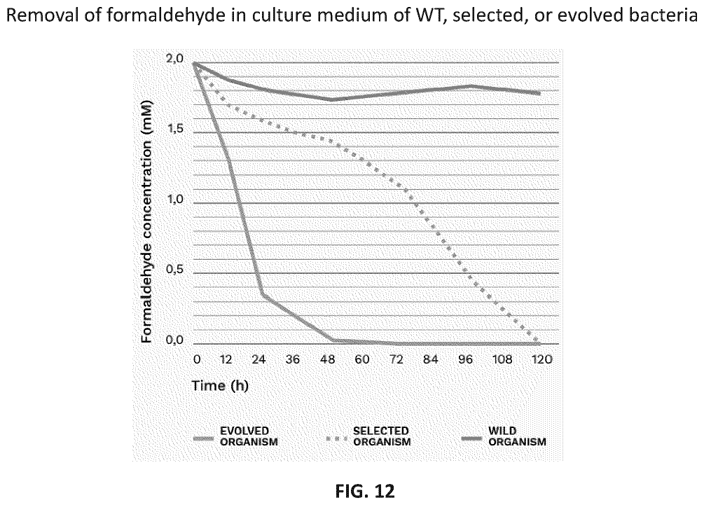

Methylobacterium oryzae CBMB20 were obtained or evolved (described in Example 7) and said microbes formaldehyde biodegradation rates were assayed in triplicate in glass tubes comprising at least 5 mL of mineral media (MP) with loose caps to facilitate oxygen exchange. Every 12 hours, 50 μL of culture was sampled and added to a spetrophotometry plate, where 50 μL of perchloric acid, and 100 μL of NASH reagent were added. The plate was incubated at about 60° C. for about 5 minutes and immediately read in a spectrophotometer (e.g., a Biotek Epoch2) at a wavelength of 400 nm. The absorbance levels of a control series of known formaldehyde concentrations was done in parallel to allow correlation of absorbance and formaldehyde concentration. Results are shown in FIG. 11 and FIG. 12.

Microbes isolated from plants and/or soil as described in Example 7 were tested and characterized using a pollutant (e.g., BTEX) as the sole carbon source(s). Microbes were streaked or spotted onto suitable growth media (e.g., minimal media agar plates) and incubated in an air-tight chamber. BTEX was added to said chamber at 2 mM each. Microbes were grown for two weeks, and growth was qualitatively assessed visually, the results of which are depicted in Table 4.

TABLE 4
Microbial Isolates Growth on BTEX

| Isolate | Origin | Growth (qualitative) |
|---|---|---|
| Pi6 | Pothos Leaf Endophyte | |
| Pi8 | Pothos Shoot Epiphyte | Faint |
| Pi12 | Pothos Shoot Endophyte | Faint |
| Pi16 | Pothos Root Endophyte | Faint |
| Pi17 | Pothos Root Endophyte | Very Faint |
| Pi18 | Pothos Root Endophyte | Yes |
| Pi19 | Pothos Root Epiphyte | Faint |
| Pi24 | Pothos Root Endophyte | Yes |
| Pi27 | Pothos Root Endophyte | Yes |
| Pi32 | Pothos Root Epiphyte | Yes |
| Pi35 | Pothos Leave Epiphyte | Faint |
| Pi36 | Pothos Root Epiphyte | Faint |
| Pi37 | Pothos Root Endophyte | Very Faint |
| Pi38 | Pothos Root Endophyte | Very Faint |
| Pi39 | Pothos Root Endophyte | Yes |
| Pi40 | Pothos Root Endophyte | Yes |
| Pi41 | Pothos Root Epiphyte | Very Faint |
| Pi42 | Pothos Root Epiphyte | Faint |
| SS2_1 | Polluted Soil | Faint |
| SS2_2 | Polluted Soil | Faint |

Fungal strains were obtained from the Fungal Biodiversity Center (CBS) and were tested and characterized using a pollutant (e.g., Benzene, Toluene, or Xylene) as the sole carbon source. Microbes were placed as plugs onto suitable growth media (e.g., minimal media agar plates) and incubated in an air-tight chamber. Benzene, Toluene, or Xylene was added to each respective chamber at 5 mM. Microbes were grown for one month, and growth was quantitatively assessed visually, the results of which are depicted in Table 5.

TABLE 5
Select Fungal Strain Radial Growth
on Benzene, Toluene, or Xylene.

| | | Radial Growth (mm) | | |
|---|---|---|---|---|
| Strain | Organism | Benzene | Toluene | Xylene |
| Ex110555 | Exophiala | 4 | 4 | 4 |
| (CBS110555) | xenobiotica | | | |
| Ex117754 | Exophiala | 6 | 5 | 1 |
| (CBS117754) | xenobiotica | | | |
| Hr176.62 | Hormoconis | 2 | 2 | 2 |
| (CBS177.62) | resinae | | | |
| Hr177.62 | Hormoconis | 1 | 1 | 1 |
| (CBS177.62) | resinae | | | |
| 1C1i110551 | Cladophialophora | 0.25 | 0.15 | 0.08 |
| (CBS110551) | immunda | | | |
| Cp0.110553 | Cladophialophora | 6 | 12 | 6 |
| (CBS110553) | psammophila | | | |
| Cs114326 | Cladosporiulm | — | — | — |
| (CBS114326) | sphaerospermum | | | |
| Pr291.30 | Picnidiella | 3 | 3 | 3 |
| (CBS291.30) | resinae | | | |
| Pv115145 | Paecilomyces | 1 | 3 | 1 |
| (CBS115145) | variotii | | | |
| Pz110552 | Pseudoeurotium | 2 | 2 | 3 |
| (CBS110552) | zonatum | | | |

### Example 7: Directed Evolution of Microorganisms

The current Example relates to directed evolution of, random mutagenesis of, and/or characterization of microbes suitable for microbiome colonization of certain compositions (e.g., plant tissues, and/or soil/media) described herein. Such a process of directed evolution may comprise a step-by-step increase of selective pressure. Such a process may occur manually, or may be performed using an automated system (e.g., the Chi.bio aka Morpheus system).

Optionally, prior to directed evolution, a microbial species and/or strain of interest may undergo a preliminary characterization for pollutant metabolism characteristics, e.g., Formaldehyde and/or BTEX biodegradation characteristics as described in Example 8.

In some methods comprising directed evolution, microbes of interest (e.g., those described herein) are serially inoculated in a series of liquid media (e.g., liquid mineral media (MMB/MP)) that have incremental increases in pollutant concentrations (e.g., Formaldehyde, and/or BTEX etc.). In some embodiments, increases in pollutant concentration occur at known levels (e.g., 2 mM, 4 mM, 6 mM, 8 mM, or 10 mM steps). Microbes may be inoculated and incubated with optimal growth medium (e.g., containing a carbon source) with added pollutants (e.g., Formaldehyde, and/or BTEX etc.). Alternatively, microbes may be inoculated and incubated with minimal growth medium (e.g., without a carbon source) with added pollutants (e.g., Formaldehyde, and/or BTEX etc.) acting as the sole carbon source. Pollutant concentrations start at or above the last known tolerance for a particular microbial strain; following inoculation, microbes are incubated until growth appears. In some methods of directed evolution, an optional mutagenesis step (e.g., UV mutagenesis) occurs before and/or during an inoculation in a stepwise pollution concentration increasing media. Following growth appearance, microbes are permitted to expand exponentially, and microbes of interest with potential biodegradation capabilities are were singled (e.g., by streaking on rich medium (CASO) with or without continued selective pressure), selected, isolated and banked for future use and/or characterization. In some methods, such a process may be repeated as many times as desired (e.g., 3, 6, 9, 12, 15, 20, 25, 30, etc.), or until a pollutant concentration is reached that completely inhibits microbial growth.

Following a stepwise round of inoculations (e.g., after 1 round, 2 rounds, 3 rounds, 4 rounds, 5 rounds, 6 rounds, 7 rounds, 8 rounds, 9 rounds, 10 rounds, 11 rounds, 12 rounds, 13 rounds, 14 rounds, 15 rounds, or more than 15 rounds; there is no limit on the number of rounds that can be performed), microbes can be isolated for characterization of their potential pollutant metabolism characteristics, e.g., Formaldehyde and/or BTEX biodegradation characteristics as described in Example 6. These characteristics can then be compared with a preliminary and/or prior characterization. Microbes with improved biodegradation characteristics are produced.

Prior to directed evolution, microbial species/strain Methylobacterium extorquens PA1, and Methylobacterium oryzae CBMB20 underwent a preliminary characterization for pollutant metabolism characteristics, e.g., VOC biodegradation characteristics as described in Example 6 (e.g., as found in Table 4, Table 5, Table 6, and Table 7).

Microbial species/strain Methylobacterium extorquens PA1, and Methylobacterium oryzae CBMB20 were serially inoculated in a series of liquid media (e.g., liquid mineral media (MMB/MP)) that had incremental increases in pollutant concentrations e.g., formaldehyde. Increases in pollutant concentration occurred at known levels (e.g., 2 mM, 4 mM, 6 mM, 8 mM, or 10 mM steps). Microbes were inoculated and incubated with minimal growth medium (e.g., without a carbon source) with added pollutants (e.g., Formaldehyde) acting as the sole carbon source. Pollutant concentrations started at or above the last known tolerance for each particular microbial strain (see Table 6); following inoculation, microbes were incubated until growth appeared. Two experimental approaches were taken, one series of pollutant concentration increases were performed without an exogenously supplied mutagen, while another series of pollutant concentration increases were performed with an exogenously supplied mutagen (e.g., UV mutagenesis). Following growth appearance, microbes were permitted to expand exponentially, and microbes of interest with potential biodegradation capabilities were singled by streaking on rich medium (CASO), selected, isolated, and banked for future use and/or characterization. Such a process was repeated at least 9 or 10 times respectively (see Table 6), and continued directed evolution can occur. Exemplary formaldehyde biodegradation performed by a Methylobacterium oryzae CBMB20 strain evolved through 4 rounds of inoculation is shown in FIG. 11 (measured using a recurrent NASH assay as described in Example 6). Such a strain had a maximum tolerance to formaldehyde of 12 mM, significantly higher than the 4 mM concentration tolerated by the strain prior to directed evolution.

TABLE 6
Select Microbial Strain Directed Evolution
for Formaldehyde Biodegradation.

| | Methylobacterium | Methylobacterium |
|---|---|---|
| | extorquens PA1 | oryzae CBMB20 |
| Initial CH20 | 6 mM | 4 mM |
| Tolerance (mM) | | |
| Rounds of Directed Evolution (DE) | 10 | 9 |
| Maximum CH₂O Tolerance after | 40 mM (6.7X) | 30 mM (7.5X) |
| DE without UV mutagenesis | | |
| Maximum CH₂O Tolerance after | 36 mM (6X) | 28 mM (7X) |
| DE with UV mutagenesis | | |

Microbial species/strain Pseudomonas putida F1, and SS2_4 (isolated herein) were serially inoculated in a series of liquid media (e.g., liquid mineral media (MMB/MP)) that had incremental increases in pollutant concentrations e.g., Benzene, Toluene, or Xylene. Increases in pollutant concentration occurred at known levels (e.g., 2 mM, 4 mM, 6 mM, 8 mM, or 10 mM steps). Microbes were inoculated and incubated with minimal growth medium (e.g., without a carbon source) with added pollutants (e.g., Benzene, Toluene, or Xylene.) acting as the sole carbon source. Pollutant concentrations started at or above the last known tolerance for each particular microbial strain (see Table 7); following inoculation, microbes were incubated until growth appeared. A series of pollutant concentration increases were performed without an exogenously supplied mutagen. Following growth appearance, microbes were permitted to expand exponentially, and microbes of interest with potential biodegradation capabilities were selected (performed using growth media with low level atmospheric BTEX concentrations (5 mM)), isolated, and banked for future use and/or characterization. Such a process was repeated at least 5, 6, 7, 8, 9, 10, 11, 12, or more times respectively (see Table 7), and continued directed evolution can occur.

TABLE 7
Select Microbial Strain Directed Evolution
for Formaldehyde or BTEX Tolerance

| | | Initial | | Current |
|---|---|---|---|---|
| | | tolerance | Rounds of | tolerance |
| Strain | Carbon source | (mM) | DE | (mM) |
| Pseudomonas | Benzene | 14 | 10 | 26 |
| putida F1 | Toluene | 6 | 8 | 38 |
| | Xylene | 58 | 10 | 80 |
| Methylobacterium | Formaldehyde | 6 | 12 | 43 |
| extorquens PA1 | | | | |
| Methylobacterium | Formaldehyde | 4 | 10 | 33 |
| oryzae CBMB20 | | | | |

### Example 8: Horizontal Transfer of Beneficial Genes

The current Example relates to the discovery of genetic loci causative of pollutant biodegradation phenotypes, and the subsequent horizontal transfer of said genes to alternative microbiome components.

An evolved strain is created as described in Example 7. Following and/or during phenotypic analysis, underlying genetic modifications are identified using an appropriate sequencing technique (e.g., full genome sequencing, whole exome sequencing, selective loci sequencing, etc.). Evolved strains genetic background are compared to wild type strains, and evolved sequences are identified. Evolved sequences are isolated and cloned for further analysis. Certain evolved sequences may provide desirable phenotypes such as efficient pollutant biodegradation and/or metabolism. Evolved sequences may be introduced to other microbial species through the process of horizontal gene transfer as is known in the art.

An environmental sample is taken from a location that may have microbes with relevant metabolic activities. In some cases, populations of microbes that may have desirable phenotypes such as efficient pollutant biodegradation and/or metabolism may be missed during sampling protocols as outlined in Example 5, as said microbes may not be amenable to culturing. Such an environmental sample can be analyzed using metagenomics, e.g., the genomic profiling of the entire sample without and/or with minimal intermediate culturing steps or manipulation. Metagenomics profiling is performed using next-generation sequencing technologies (e.g., Illumina based shotgun sequencing, Illumina MiSeq, etc.) coupled with metagenome assembly tools (e.g., SOAPdenovo2, MOCAT, MetAMOS, SPAdes Assembler, Check-M, Harvest, MUMmer, Prokka, MLST_Check, etc.), and annotation where necessary. Alternatively or in tandem, metagenomics analysis is performed using 16S/ITS sequencing to identify phylogenetic relationships. Metagenomic analysis facilitates identification of previously non-isolated strains that may be of interest. Following identification of sequences of interest, microbes can be resampled using optimized collection and/or culturing techniques, or sequences of interest can be cloned using synthetic biology.

Samples are obtained from a variety of common house plants, in a variety of conditions (e.g., well maintained, poorly maintained, with other plants, in isolation etc.). Samples are taken from plant surfaces, tissues, and soils as described in Example 6. New strains are identified that may comprise genes that bestow phenotypic characteristics of interest (e.g., efficient pollutant biodegradation), and/or strains are identified that are considered hardy and/or non-pathogenic that are amenable to horizontal gene transfer. Genes of interest can be identified, and either cloned or created using synthetic biology.

Wild type and evolved strains are co-cultured with or without slight or stringent selective pressure. In cases where an evolved strain has lost fitness when compared to a wild type strain, co-culturing and/or co-cultivation can permit natural horizontal gene transfer and creation of an intermediate hybrid strain that may provide certain evolved and wild type characteristics. In some cases, wild type strains are provided with lysed evolved strains and/or isolated evolved strain genetic information. In certain embodiments, wild type strains are transformed with certain evolved sequences, rendering a wild type strain engineered and potentially providing a wild type strain with certain evolved and desirable characteristics (e.g., efficient pollutant biodegradation).

### Example 9: Plant-Microorganism Interface and Microbiome Management

The current Example relates to the interaction between compositions described herein, e.g., between plants and their microbiome.

A microorganism of interest is identified and/or created (e.g., see Examples 5-8). Said microbe is suspended in a suitable solution (e.g., MgSO4 10 mM with Tween 20 at 0.01%) and inoculated onto a naïve plant (e.g., through submersion, spraying or other suitable method) and/or a suitable media (e.g., soil, hydroponic water, activated charcoal, a container etc.). An inoculated plant is visually monitored for a suitable period of time (e.g., 1 day, 2 days, 1 week, 2 weeks, 4 weeks, 2 months, 6 months, 1 year, etc.) for microbe induced symptoms (e.g., necrosis, growth defects, etc.). An inoculated plant is tested for pollutant biodegradation (e.g., formaldehyde, methanol, and/or BTEX etc.), and kinetics of pollutant biodegradation within an air-tight enclosure are measured using an integrated formaldehyde, methanol, and/or BTEX sensor capable of monitoring a pollutant's concentration over time. Long term survival and colonization of a plant by a newly introduced microbe are measured, where a microbe of interest is re-isolated (e.g., as described in Example 5) after a suitable period of time (e.g., 1 week, 2 weeks, 4 weeks, 2 months, 6 months, 1 year, etc.). A microbe of interest is selected for by inoculating isolates in mineral media comprising a known stringent concentration of pollutant (e.g., maximum pollutant tolerance level as described in Example 8). Long term survival and colonization of a plant by a newly introduced microbe is confirmed. A stable interaction is formed.

A composition of interest (e.g., a plant, a microbe, and/or a combination thereof) is placed within an air-tight container, where a plant stem passes through a PTFE septum. Such a system facilitates pollutant degradation assessment performed by a plants aerial organs and/or a plants phyllosphere.

A plant and microbe combination can have an enhanced microbiome. Such an enhanced microbiome can comprise an engineered microbe coupled with compounds useful for bacterial growth and/or stabilization of growth conditions (e.g., pH optimization, heavy metals availability, F/BTEX degradation elicitors, selection against other bacterial populations etc.).

Certain microbes described herein that are shown to improve a depollution capacity of various indoor plants, (e.g., MePA1, MoCBM, PpF1 and/or SS2-2) were not directly isolated from Pothos. In certain cases, such a plant and microbe interaction is likely not specific, and such a microbe may be amenable for compositions comprising a plant other than Pothos. Alternatively, a composition can be produced that includes such a microbe without a host plant. Such a composition can be administered to a variety of indoor plants as a supplement.

Microorganism of interest such as MePA1 MePA1, MoCBM, PpF1 and/or SS2-2, were identified and/or created (e.g., see Examples 5-8). Said microbes were individually suspended in a suitable solution (e.g., MgSO4 10 mM with Tween 20 at 0.01%) and inoculated onto a naïve plant (e.g., through spraying). An inoculated plant was visually monitored for a suitable period of time (e.g., up to 6 months) for microbe induced symptoms (e.g., necrosis, growth defects, etc.). Microbes were qualitatively found to be non-toxic. An inoculated plant was tested for pollutant biodegradation (e.g., formaldehyde, methanol, and/or BTEX etc.), and kinetics of pollutant biodegradation within an air-tight enclosure were measured using an integrated formaldehyde, methanol, and/or BTEX sensor capable of monitoring a pollutant's concentration over time. Long term survival and colonization of a plant by a newly introduced microbe was measured, where a microbe of interest was re-isolated (e.g., as described in Example 5) after a suitable period of time (e.g., 2 week, 4 weeks, 6 weeks, 9 weeks, and 12 weeks). A microbe of interest was selected for by inoculating isolates in mineral media comprising a known stringent concentration of pollutant (e.g., maximum pollutant tolerance level as described in Example 6 and Example 7). Long term survival and colonization of a plant by a newly introduced microbe was confirmed. A stable interaction was formed (see Table 8).

TABLE 8
Select Microbial Strain Directed Evolution
for Formaldehyde Biodegradation.

| | | Post-Inoculation Resampling for Strain Presence | | | | |
|---|---|---|---|---|---|---|
| Strain | Substrate | 2 weeks | 4 weeks | 6 weeks | 9 weeks | 13 weeks |
| MePA1 | Soil | Yes | Yes | Yes | Yes | Yes |
| | Leaves | NA | Yes | No | No | No |
| MoCBM | Soil | Yes | Yes | Yes | No | No |
| | Leaves | NA | Yes | No | No | No |
| PpF1 | Soil | Yes | Yes | Yes | Yes | Yes |
| | Leaves | No | No | No | No | No |
| SS2_4 | Soil | Yes | Yes | Yes | Yes | Yes |
| | Leaves | Yes | Yes | No | No | No |

An inoculated plant was tested for pollutant biodegradation (e.g., benzene), and the kinetics of pollutant biodegradation were measured using an air-tight enclosure with an integrated formaldehyde and/or BTEX sensor capable of monitoring a pollutant's concentration over time (e.g., as described in Example 4). Benzene concentration (ppm) was measured in closed containers comprising plants with evolved microbiomes compared to those with a native microbiome. Plants with an evolved microbiome showed significant reductions in aerosolized benzene when compared to control plants with a native microbiome (See FIG. 14A).

An inoculated plant was tested for pollutant biodegradation (e.g., toluene), and the kinetics of pollutant biodegradation were measured using an air-tight enclosure with an integrated formaldehyde and/or BTEX sensor capable of monitoring a pollutant's concentration over time (e.g., as described in Example 4). Toluene concentration (ppm) was measured in closed containers comprising plants with evolved microbiomes compared to those with a native microbiome. Plants with an evolved microbiome showed an ability to significantly reduce aerosolized toluene when compared to control plants with a native microbiome (See FIG. 13A).

### Example 10: Characterization of Microbes

The present Example confirms that, as described herein, plants (e.g., Epipremnum aureum plants) inoculated with microbes may have enhanced pollutant (e.g., formaldehyde, benzene, toluene, and/or xylene) phytoremediation, e.g., as compared to an appropriate reference (e.g., plants with a native microbiome).

Concentrated microbes (e.g., Pseudomonas putida F1 (PpF1)) identified, as described, in Example 5-9 were prepared in a low volume (see Table 9) and suspended in a suitable solution (e.g., MgCl2). Under continuous lights, a plant (e.g., Epipremnum aureum) was inoculated with the concentrated microbe (e.g., PpF1) solution and the solution was poured on the soil of the potted plant (e.g., Epipremnum aureum). The controls (e.g., plants with a native microbiome) were given the same volume of the suitable solution (e.g., MgCl2) without microbial cultures.

An inoculated plant was tested for pollutant (e.g., formaldehyde, benzene, toluene, and/or xylene) biodegradation, and the kinetics of pollutant biodegradation were measured using an air-tight enclosure with an integrated formaldehyde and/or BTEX sensor capable of monitoring a pollutant's concentration over time (e.g., as described in Example 4)

TABLE 9
Experimental Conditions for Bacteria Concentration

| | Volume of | OD in a |
|---|---|---|
| Pollutant | Concentrated | suitable solution |
| Experiment | Microbe | (e.g., MgCL2) |
| Benzene | 10 mL | 11.6 |
| Toluene | 10 mL | 11.6 |
| Xylene | 5 mL | 34.6 |
| Formaldehyde | 1 mL | 10 |

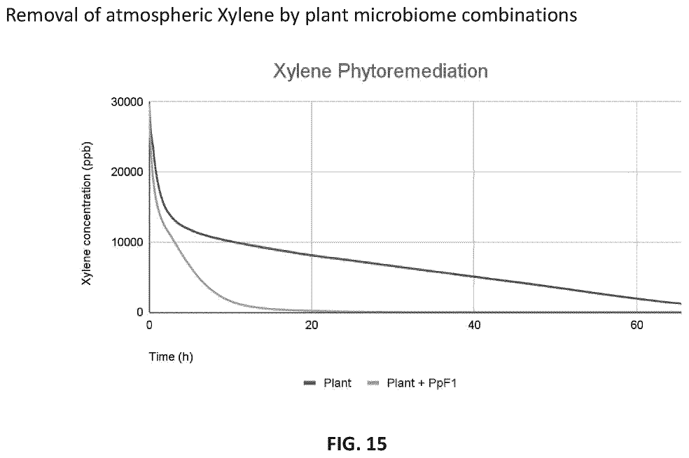

Among other things, the present Example demonstrates that a plant (e.g. Epipremnum aureum plant) with an evolved microbiome (e.g., PpF1) may have enhanced pollutant (e.g., Benzene, Toluene, and/or Xylene) phytoremediation, e.g., as compared to an appropriate reference (e.g., plant with a native microbiome) (FIG. 13B, FIG. 14B, and/or FIG. 15). Specifically, in this Example, inoculation of a plant (e.g. Epipremnum aureum plant) with a microbe (e.g., PpF1) increased pollutant (e.g., Benzene, Toluene, and/or Xylene) degradation speed by at least 9×, e.g., as compared to an appropriate reference (e.g., plants with a native microbiome) (FIG. 13B, FIG. 14B, and/or FIG. 15). In some embodiments, a plant (e.g. Epipremnum aureum plant) with a microbe (e.g., PpF1) may exhibit increased pollutant (Benzene, Toluene, and/or Xylene) phytoremediation within 12 hours, 24 hours, 48 hours, and/or 60 hours (FIG. 13B, FIG. 14B, and/or FIG. 15). In some embodiments, a plant (e.g. Epipremnum aureum plant) with a microbe identified as in Examples 5-9 may have enhanced pollutant (e.g., formaldehyde, benzene, toluene, ethylbenzene and/or xylene) phytoremediation, e.g., as compared to an appropriate reference (e.g., plants with a native microbiome).

In another experiment, pollutant (e.g., formaldehyde) degradation was measured using plants (e.g. Epipremnum aureum plants) inoculated with concentrated microbes (e.g., Methylobacterium extorquens PA1 (MePA1), Methylobacterium oryzae CBMB20 (MoCBM) and/or Pseudomonas putida F1 (PpF1)) identified in Example 5-9. The concentrated microbes (e.g., Methylobacterium extorquens PA1 (MePA1), Methylobacterium oryzae CBMB20 (MoCBM) and/or Pseudomonas putida F1 (PpF1)) were prepared in a low volume (see Table 9) and suspended in suitable solution (e.g., MgCl2).

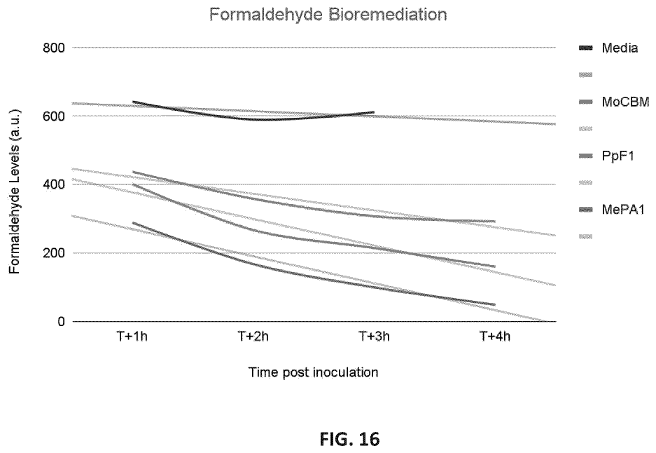

Among other things, the present Example further demonstrates that plants (e.g. Epipremnum aureum plants) inoculated with concentrated microbes may have enhanced pollutant (e.g., formaldehyde) phytoremediation, e.g., as compared to an appropriate reference (e.g., plants with a native microbiome) (FIG. 16). Specifically, in this Example, as demonstrated in FIG. 16, inoculation of a plant (e.g. Epipremnum aureum plant) with MoCBM, PpF1, or MePA1 increased pollutant (e.g., formaldehyde) degradation speed by at least 3.2×, 5.1×, and 5.2× respectively, e.g., as compared to an appropriate reference (e.g., plants with a native microbiome). In some embodiments, as demonstrated in FIG. 16, Epipremnum aureum plants inoculated with an evolved microbiome (e.g., MoCBM, PpF1, and/or MePA1) may exhibit increased pollutant (e.g., formaldehyde) phytoremediation within 1 hour, 2 hours, 3 hours, and/or 4 hours post inoculation e.g., as compared to an appropriate reference (e.g., plants with a native microbiome).

In some embodiments, Epipremnum aureum plants inoculated with an evolved microbiome (e.g., MoCBM, PpF1, and/or MePA1) may exhibit increased pollutant (e.g., benzene, toluene, ethylbenzene and/or xylene) phytoremediation e.g., as compared to an appropriate reference (e.g., plants with a native microbiome).

### Example 11: Stability of Engineered Microbes

The present Example confirms that, as described herein, engineered microbiome may enhance pollutant biodegradation (e.g., toluene) of a plant (e.g., Epipremnum aureum) over an extended period (e.g., several weeks) as compared to an appropriate reference (e.g, plants with a native microbiome).

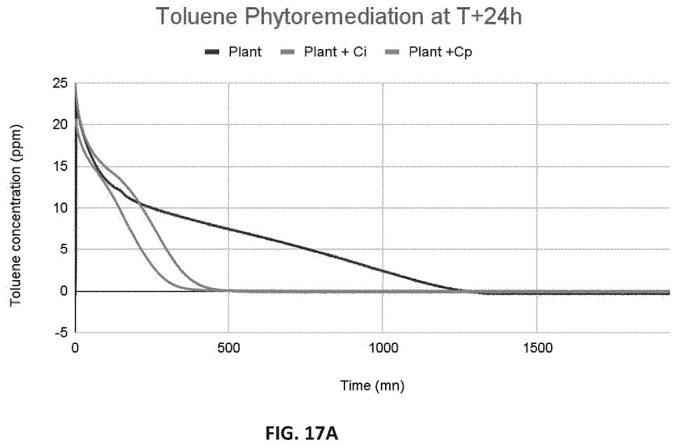

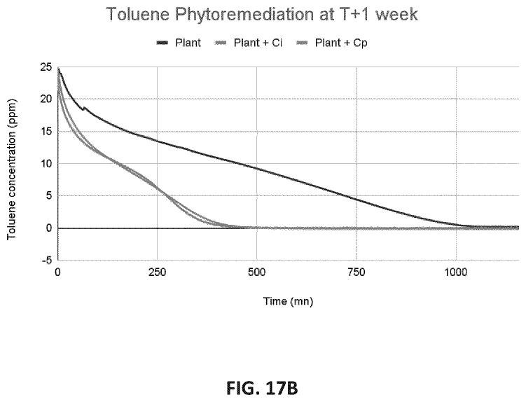

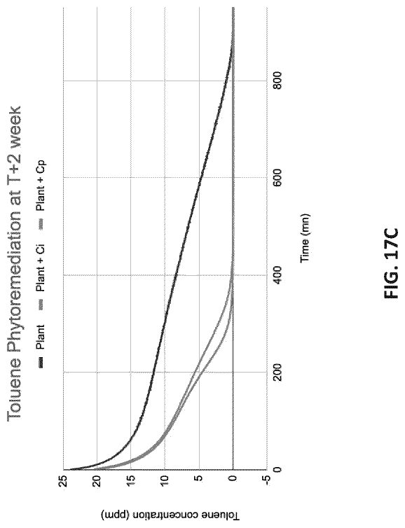

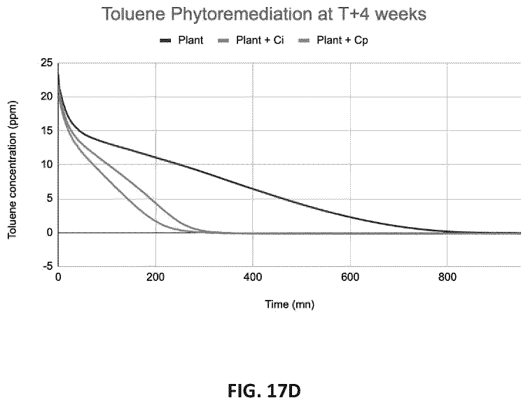

Plants (e.g. Epipremnum aureum plants) were inoculated with mature cultures of microbes (e.g., 1Cli110551 (CBS110551) and/or Cp0.110553 (CBS110553)) on agar plates. The mycelium was gathered using a spatula to minimize the amount of agar media. The mycelium was placed in a falcon containing 20 tungsten beads and 20 mL of 10 mM MgCl2, and then disrupted for 15 minutes on a vortex at moderate setting. Once disrupted, 10 mL of the mycelium culture was added to a potted Epipremnum aureum. The toluene phytoremediation capacity of the resulting plants were measured at 24 hours (FIG. 17A), 1 week (FIG. 17B), 2 weeks (FIG. 17C) and 4 weeks (FIG. 17D) post-inoculation.

Among other things, the present Example demonstrates that plants (e.g., Epipremnum aureum plants) with engineered microbiomes may have enhanced pollutant (e.g., toluene) biodegradation over an extended period (e.g., several weeks) as compared to an appropriate reference (e.g., plants with a native microbiome) (FIG. 17A-D). In some embodiments, as demonstrated in FIGS. 17A-D, an engineered microbe (e.g., 1C1i110551 (CBS110551) and/or Cp0.110553 (CBS110553)) may enhance pollutant (toluene) biodegradation of a plant for at least 1 week, 2 week, 3 week, and/or 4 weeks e.g., as compared to an appropriate reference (e.g., plants with a native microbiome). In some embodiments, as demonstrated in FIGS. 17A-D, pollutant (e.g., toluene) degradation speed was increased by at least by 4.6× and 4.9× after 24 h, 3× and 2.4× after 1 week, 2.5× and 2× after 2 weeks, 2.5× and 2.8× after 4 weeks, post-inoculation of Epipremnum aureum with 1C1i110551 (CBS110551) and Cp0.110553 (CBS110553) respectively, e.g., as compared to an appropriate reference (e.g., plants with a native microbiome). In some embodiments, as demonstrated in FIG. 17A, an engineered microbe (e.g., 1C1i110551 (CBS110551) and/or Cp0.110553 (CBS110553))) may enhance pollutant (toluene) biodegradation of a plant within 9 hours post inoculation e.g., as compared to an appropriate reference (e.g., plants with a native microbiome).

In some embodiments, Epipremnum aureum plants with engineered microbiomes, as described herein, may increase pollutant biodegradation (e.g., benzene, ethylbenzene, xylene, and/or formaldehyde) over an extended period (e.g. several weeks) e.g., as compared to an appropriate reference (e.g., plants with a native microbiome).

### Example 12: Pollutant Phytoremediation of Transgenic Plants

The present Example confirms that, as described herein, transgenic plants comprising a gene of interest may have enhanced pollutant (e.g., formaldehyde and/or BTEX) phytoremediation as compared to a reference (e.g. a non-transgenic plant). Among other things, and as discussed herein, the present disclosure provides an insight that synthetic metabolic pathways (e.g., as disclosed herein) may be applied to (e.g., engineered into) plants, and specifically into ornamental plants. Without wishing to be bound by any theory, the present disclosure proposes that such, metabolic pathways may affect central metabolism pathways that are conserved between or among plant species.

The present Example demonstrates introduction of synthetic metabolic pathway(s) into a model plant (specifically Arabidopsis thaliana), and establishes proof of concept for technologies as described herein. The present disclosure further explains applicability of this finding to other plant species, including specifically to other ornamental plant species, and establishes that pathway engineering as described herein may be utilized to enhance pollutant phytoremediation in various plant species, an in particular in various ornamental plants.

Exemplary constructs comprising a gene of interest (see Table 10) were transformed into plants (e.g., model plant such as Arabidopsis thaliana) to modify a pollutant (e.g., formaldehyde and/or BTEX) metabolism via a synthetic pathway (See Table 10). Methods for transformation and selection are disclosed herein (see, e.g., Example 2) and/or are known in the art.

TABLE 10
Synthetic Pathway and Gene of Interest

| Pathway | Gene 1 | Gene 2 |
|---|---|---|
| RumP | HPS/PHI_a | |
| | HPS_Bm | PHI_Bm |
| | HPS_Mg | PHI_Mg |
| XuMP | DAS_Canbo | DHAK_Sc |
| | DAS_Canbo | DHAK_Ec |
| Serine | FALDH_Ea | FDH |
| BTEX | TodC1 | |
| | PhOH | |

To measure phytoremediation, transgenic plants were placed in a 2 L glass jar and exposed to high levels of a pollutant (e.g., formaldehyde and/or BTEX) for at least 24 hours. A plant was tested for pollutant biodegradation (e.g., formaldehyde and/or BTEX) and/or kinetics of pollutant biodegradation (e.g., formaldehyde and/or BTEX) by using an air-tight enclosure with an integrated formaldehyde and/or BTEX sensor capable of monitoring a pollutant's concentration over time (e.g., as described in Example 4). The gaseous concentration of the pollutant (e.g., formaldehyde and/or BTEX) was measured before and after this exposure, then results were normalized by leaf surface area.

Pathway metabolomics were measured by placing transgenic plants in a 2 L jar with 0 mM or at least 5 mM pollutant (e.g. formaldehyde) for at least 18 hours. After exposure, leaves were excised and extracted for detection of fructose and/or Gycline via GC-MS analysis. Fructose, a downstream product of the XuMP pathway, and Glycine, a downstream product of the Serine pathway, were measured.

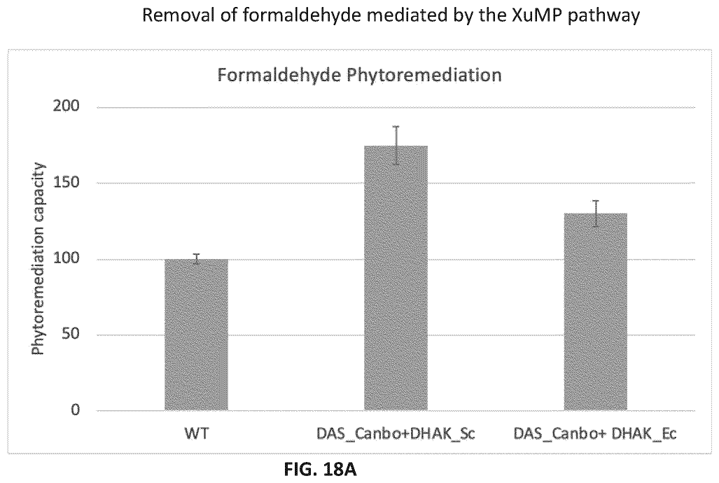

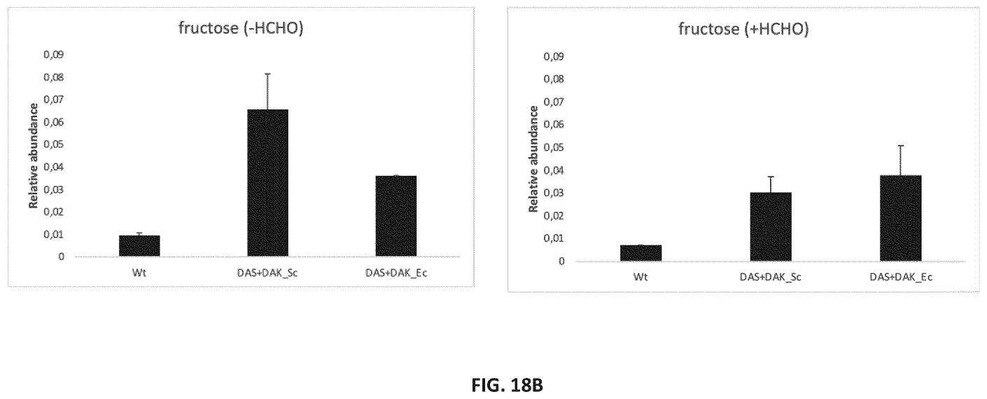

Among other things, the present Example confirms that, as described herein, transgenic plants as described herein may have increased removal of formaldehyde mediated by the XuMP pathway, e.g., as compared to an appropriate reference (e.g., a non-transgenic plant). Specifically, in this Example, as demonstrated in FIGS. 18A and 18B, in the particular exemplified engineered plants, formaldehyde phytoremediation capacity was increased at least about 25% (FIG. 18A) and/or fructose relative abundance was increased by at least 50% (FIG. 18B), e.g., as compared to an appropriate reference (e.g., a non-transgenic plant). In some embodiments, a transgenic plant with heterologous expression of a DAS enzyme and a DHADK_Sc enzyme may have increased formaldehyde phytoremediation and/or fructose metabolism when compared to a transgenic plant with heterologous expression of a DAS enzyme and a DHADK_Ec enzyme.

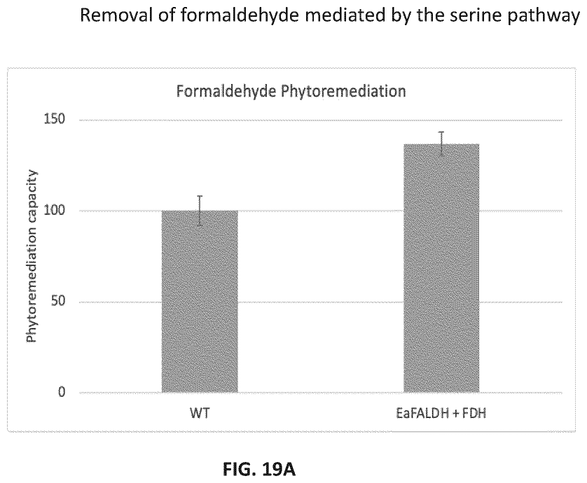

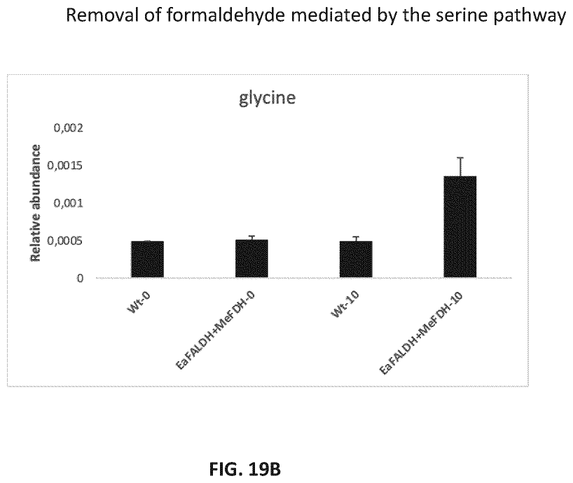

Among other things, the present Example confirms that, as described herein, transgenic plants may have increased removal of formaldehyde mediated by the serine pathway as compared to an appropriate reference (e.g., a non-transgenic plant). Specifically, in this Example, as demonstrated in FIGS. 19A and 19B, in the particular exemplified engineered plants, formaldehyde phytoremediation capacity was increased at least about 25% (FIG. 19A) and/or glycine relative abundance was increased by at least 50% (FIG. 19B), e.g., as compared to an appropriate reference (e.g., a non-transgenic plant).

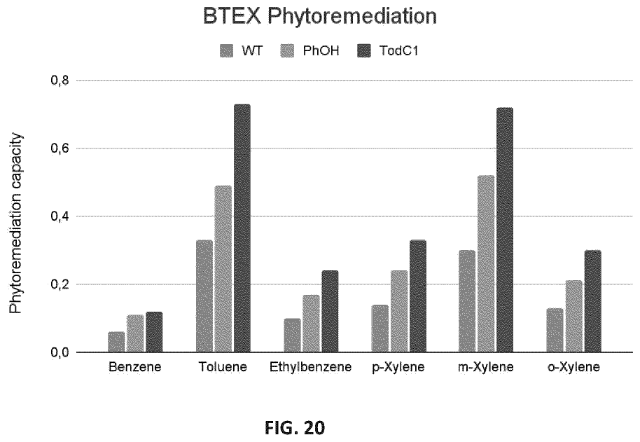

Among other things, the present Example confirms that, as described herein, transgenic plants may have increased BTEX phytoremediation as compared to a reference (e.g., non-transgenic plant). In some embodiments, as demonstrated in FIG. 20, a heterologous expression of a PhOH enzyme and/or a TodClenzyme in a transgenic plant may increase BTEX phytoremediation capacity of the plant, e.g., as compared to an appropriate reference (e.g., a non-transgenic plant). In some embodiments, a transgenic plant, as described herein, may induce production of muconic acid.

### Example 13: Stomatal Density Optimization

The present Example demonstrates that, among other things, plants may be engineered to express (e.g., to overexpress) a gene that may increase stomatal density and/or pollutant phytoremediation (e.g., formaldehyde). Among other things, the present disclosure provides an insight that such engineering may be applied to ornamental plants to increase stomata formation. Without wishing to be bound by any theory, the present disclosure proposes in particular that such engineering can desirably be applied to a gene that is conserved between ornamental plants. In some embodiments, the methods developed herein to increase stomata formation may enhance pollutant phytoremediation. One particularly useful feature of certain embodiments of this aspect of the present disclosure is its potential applicability across a variety of plant species.

Exemplary constructs (see Table 2) were transformed (e.g., as described in Example 2) into model plants (e.g., Arabidopsis thaliana) and rate of influx of volatile organic compounds into the plant was assessed. After exposure to high levels of a pollutant (e.g., formaldehyde) for at least 24 hours, engineered plants were tested for pollutant biodegradation (e.g., formaldehyde)

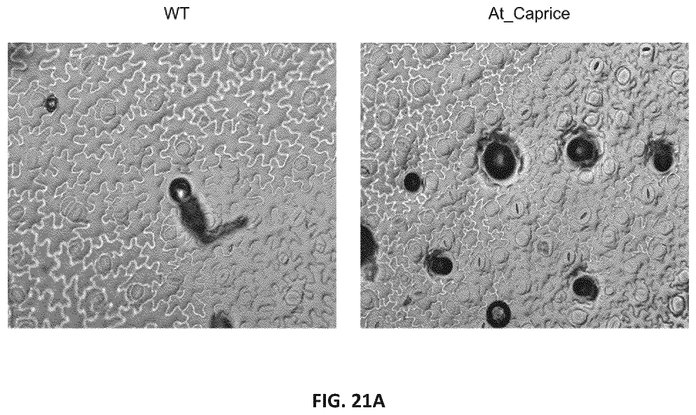

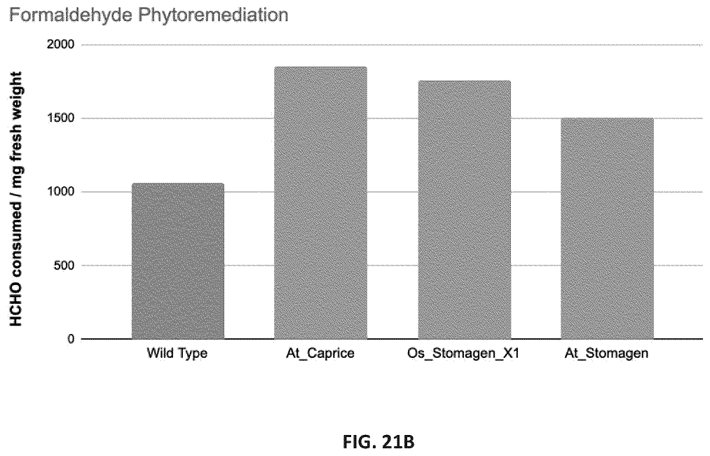

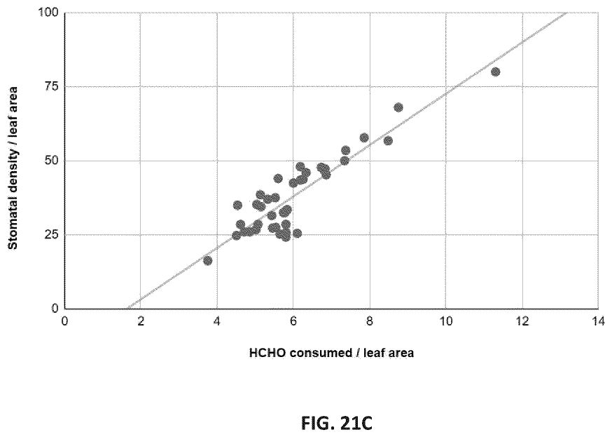

Among other things, the present Example demonstrates that plants engineered to express (e.g., to overexpress) a gene (AtCaprice, AtStomagen, and/or OsX1) may exhibit increased stomatal density and/or pollutant phytoremediation (e.g., formaldehyde). In some embodiments, as demonstrated in FIG. 21A, an engineered plant, as described herein, may increase leaf stomatal density. In some embodiments, as demonstrated in FIG. 21B, an engineered plant may increase rate of pollutant (e.g., formaldehyde) remediated by the plant by at least 50%, e.g., as compared to an appropriate reference (e.g., a non-transgenic plant) (FIG. 21B). In some embodiment, as demonstrated in FIG. 21C, the amount of formaldehyde remediated by a plant is correlated to stomatal density.

In some embodiments, as described herein, plants engineered to express (e.g., to overexpress) a gene (AtCaprice, AtStomagen, and/or OsX1) may exhibit increased stomatal density and/or pollutant phytoremediation (e.g., BTEX).

### Example 14: Optimization of Regulatory Elements

The present Example demonstrates that, among other things, that regulatory elements disclosed herein may be used to drive and/or increase expression of a gene and/or protein of interest.

The capacity of regulatory elements to increase expression levels of a polypeptide were measured. Leaf mesophyll cells were transformed with a construct comprising a promoter, a fluorescence reporter gene, and a terminator. Single cell fluorescence levels were measured on Epipremnum aureum leaf mesophyll cells to determine expression of the fluorescence reporter polypeptide and strong regulatory element combinations has a fluorescence score of at least 0.65.

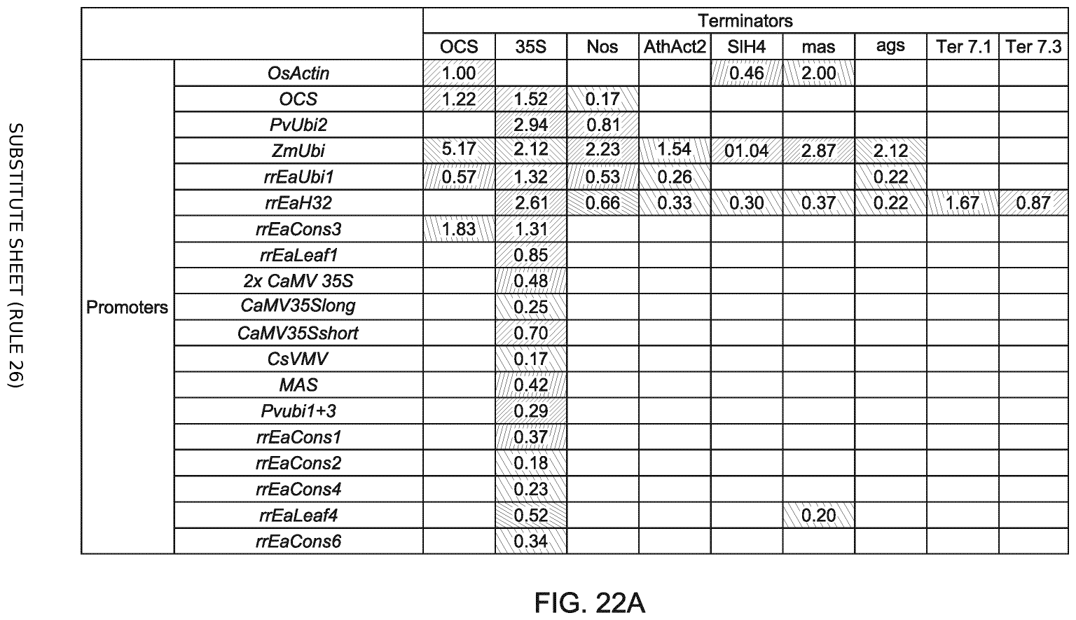

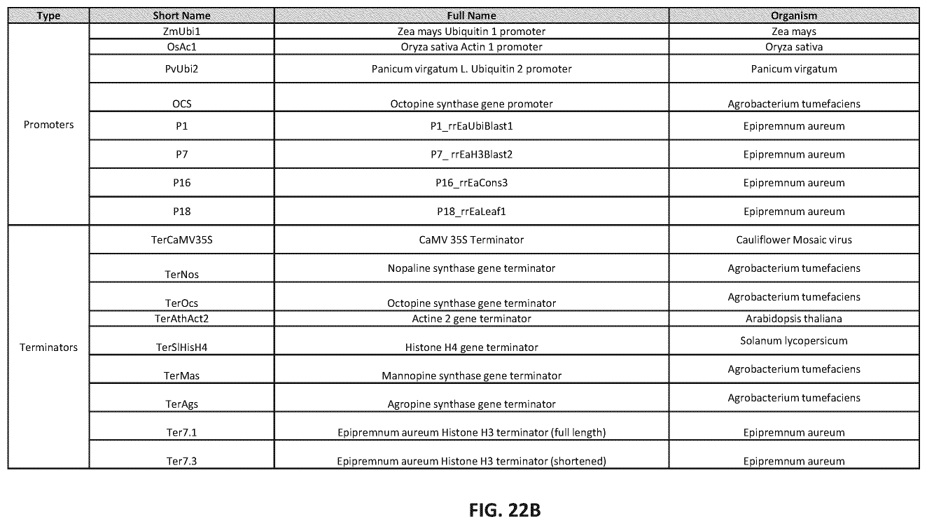

Among other things, the present disclosure demonstrates that various combinations of regulatory elements may be optimized to increase expression of an enzyme of interest. In some embodiments, as demonstrated in FIG. 22A, a construct comprising ZmUbi may increase expression of a gene of interest. In some embodiments, as demonstrated in FIG. 22A, a construct comprising PvUbi2 may increase expression of a gene of interest. In some embodiments, as demonstrated in FIG. 22A, constructs comprising a combination of promotor originating from Epipremnum aureum (e.g., rrEaUbi1, rrEaH32, rrEaCons3, and/or rrEaLeaf1) and terminators (e.g., OCS, 35S, and/or Nos) may increase expression of a gene of interest. In some embodiments, e.g., as demonstrated in FIG. 22A, constructs comprising a combination of promotor originating from Epipremnum aureum (e.g., rrEaH32) and terminators originating from Epipremnum aureum (e.g., Ter 7.1 and/or Ter 7.3) may increase expression of a gene of interest.

### EXEMPLARY EMBODIMENTS

Embodiment 1. An engineered ornamental indoor plant characterized in that:
   (a) it expresses at least one heterologous formaldehyde and/or methanol metabolism polypeptide; and
   (b) when cultivated or maintained in an environment comprising a volatile organic compound (VOC), exhibits an increased rate of air VOC removal, when compared to an ornamental indoor plant that has not been so engineered.
Embodiment 2. The engineered ornamental indoor plant of embodiment 1 that is stably transformed with at least one expression vector from which the at least one formaldehyde metabolism polypeptide is expressed.
Embodiment 3. The engineered ornamental indoor plant of embodiment 1 that is stably transformed with a plurality of expression vectors from which a plurality of formaldehyde metabolism polypeptides are expressed.
Embodiment 4. The engineered ornamental indoor plant of embodiment 1 wherein a plurality of polypeptides function in concert to chemically convert a VOC to a usable sugar substrate.
Embodiment 5. The engineered ornamental indoor plant of embodiment 1, wherein the at least one heterologous formaldehyde metabolism polypeptide comprises: 3-hexulose-6-phosphate synthase (HPS), 6-phospho-3-hexuloisomerase (PHI), dihydroxyacetone synthase (DAS), dihydroxyacetone kinase (DAK), formaldehyde dehydrogenase (FALDH), glutathione-dependent formaldehyde dehydrogenase (GSH-FALDH), glycolaldehyde synthase (GALS), acetyl-phosphate synthase (ACPS), phosphate acetyltransferase (PTA), 2-keto-4-hydroxybutyrate aldolase (KHB), branched-chain alpha-keto acid decarboxylase (KDC), pyruvate decarboxylase (PDC), NADH-dependent 1,3-PDO oxidoreductase (DhaT), non-specific NADPH-dependent alcohol dehydrogenase (YqhD), serine aldolase (SAL), threonine aldolase (LtaE), serine deaminase (SDA), 4-hydroxy-2-oxobutanoate (HOB) aldolase (HAL), HOB aminotransferase (HAT), serine hydroxymethyltransferase 1 mitochondrial (SHM1), (S)-2-hydroxy-acid oxidase (GLO1 and/or GLO2), formate dehydrogenase (FDH), and/or formolase (FLS).
Embodiment 6. The engineered ornamental indoor plant of embodiment 1, wherein the at least one heterologous formaldehyde metabolism polypeptide comprises 3-hexulose-6-phosphate synthase (HPS), and/or 6-phospho-3-hexuloisomerase (PHI).
Embodiment 7. The engineered ornamental indoor plant of embodiment 1, wherein the at least one heterologous formaldehyde metabolism polypeptide a comprises dihydroxyacetone synthase (DAS), and/or dihydroxyacetone kinase (DAK).
Embodiment 8. The engineered ornamental indoor plant of embodiment 1, wherein the at least one heterologous formaldehyde metabolism polypeptide comprises formaldehyde dehydrogenase (FALDH), glutathione-dependent formaldehyde dehydrogenase (GSH-FALDH), serine hydroxymethyltransferase 1 mitochondrial (SHM1), (S)-2-hydroxy-acid oxidase (GLO1 and/or GLO2) and/or formate dehydrogenase (FDH).
Embodiment 9. The engineered ornamental indoor plant of embodiment 1, wherein the at least one heterologous formaldehyde metabolism polypeptide comprises formolase (FLS), and/or dihydroxyacetone kinase (DAK).
Embodiment 10. The engineered ornamental indoor plant of embodiment 1, wherein the at least one heterologous formaldehyde metabolism polypeptide comprises glycolaldehyde synthase (GALS), acetyl-phosphate synthase (ACPS), and/or phosphate acetyltransferase (PTA).
Embodiment 11. The engineered ornamental indoor plant of embodiment 1, wherein the at least one heterologous formaldehyde metabolism polypeptide comprises 2-keto-4-hydroxybutyrate aldolase (KHB), branched-chain alpha-keto acid decarboxylase (KDC), pyruvate decarboxylase (PDC), NADH-dependent 1,3-PDO oxidoreductase (DhaT), and/or non-specific NADPH-dependent alcohol dehydrogenase (YqhD).
Embodiment 12. The engineered ornamental indoor plant of embodiment 1, wherein the at least one heterologous formaldehyde metabolism polypeptide comprises serine aldolase (SAL), threonine aldolase (LtaE), serine deaminase (SDA), 4-hydroxy-2-oxobutanoate (HOB) aldolase (HAL), and/or HOB aminotransferase (HAT).
Embodiment 13. The engineered ornamental indoor plant of embodiment 1, wherein prior to introduction to the ornamental indoor plant, the at least one heterologous formaldehyde metabolism polypeptide has been modified using protein evolution.
Embodiment 14. A cell or population of cells derived from the engineered ornamental indoor plant of embodiment 1.
Embodiment 15. An engineered ornamental indoor plant characterized in that:
   (a) it expresses at least one heterologous benzene, toluene, ethylbenzene, or xylene (BTEX) metabolism polypeptide; and
   (b) when cultivated or maintained in an environment comprising a volatile organic compound (VOC), exhibits an increased rate of air VOC removal when compared to an ornamental indoor plant that has not been so engineered.
Embodiment 16. The engineered ornamental indoor plant of embodiment 1 that is stably transformed with at least one expression vector from which the at least one BTEX metabolism polypeptide is expressed.
Embodiment 17. The engineered ornamental indoor plant of embodiment 15 that is stably transformed with a plurality of expression vectors from which a plurality of BTEX metabolism polypeptides are expressed.
Embodiment 18. The engineered ornamental indoor plant of embodiment 15 wherein a plurality of polypeptides function in concert to chemically convert BTEX to a usable anabolic substrate.
Embodiment 19. The engineered ornamental indoor plant of embodiment 15, wherein the at least one heterologous BTEX metabolism polypeptide comprises: cytochrome P450 monooxygenase, O-xylene monooxygenase oxygenase subunit alpha, benzene monooxygenase oxygenase subunit, toluene-4-monooxygenase system ferredoxin-NAD (+) reductase component, toluene monooxygenase alpha subunit, aromatic ring-hydroxylating dioxygenase subunit alpha, hydroxylase alpha subunit, phenylalanine hydroxylase, benzene 1,2-dioxygenase, cis-1,2-dihydrobenzene-1,2-diol dehydrogenase, toluene methyl-monooxygenase, aryl-alcohol dehydrogenase, benzaldehyde dehydrogenase (NAD+), and/or benzaldehyde dehydrogenase (NADP+).
Embodiment 20. The engineered ornamental indoor plant of embodiment 15, wherein the at least one heterologous BTEX metabolism polypeptide alters the benzene and/or ethylbenzene metabolism pathway, wherein the heterologous polypeptides comprise benzene monooxygenase oxygenase subunit, benzene 1,2-dioxygenase, and/or cis-1,2-dihydrobenzene-1,2-diol dehydrogenase.
Embodiment 21. The engineered ornamental indoor plant of embodiment 15, wherein the at least one heterologous BTEX metabolism polypeptide alters the toluene and xylene metabolism pathway, wherein the heterologous polypeptides comprise O-xylene monooxygenase oxygenase subunit alpha, toluene-4-monooxygenase system ferredoxin-NAD (+) reductase component, toluene monooxygenase alpha subunit, toluene methyl-monooxygenase, aryl-alcohol dehydrogenase, benzaldehyde dehydrogenase (NAD+) and/or benzaldehyde dehydrogenase (NADP+).
Embodiment 22. The engineered ornamental indoor plant of embodiment 15, wherein the at least one heterologous BTEX metabolism polypeptide alters the phenol and/or phenol (like) metabolism pathway, wherein the heterologous polypeptides comprise phenol hydroxylase component phP, phenol hydroxylase, and/or uncharacterized protein A4U43_C04F5180.
Embodiment 23. The engineered ornamental indoor plant of embodiment 15, wherein the at least one heterologous BTEX metabolism polypeptide alters the catechol and/or catechol (like) metabolism pathway, wherein the heterologous polypeptides comprise 3-isopropylcatechol-2,3-dioxygenase, metapyrocatechase, extradiol dioxygenase, catechol 2,3-dioxygenase, and/or catechol 1,2-dioxygenase.
Embodiment 24. The engineered ornamental indoor plant of embodiment 15, wherein prior to introduction to the ornamental indoor plant, the at least one heterologous BTEX metabolism polypeptide has been modified using protein evolution.
Embodiment 25. A cell or population of cells derived from the engineered ornamental indoor plant of embodiment 15.
Embodiment 26. The engineered ornamental indoor plant of embodiment 15, crossed with the engineered ornamental plant of embodiment 1.
Embodiment 27. The engineered ornamental indoor plant of embodiment 15, comprising the additional engineered attributes of embodiment 1.
Embodiment 28. A cell or population of cells derived from the engineered ornamental indoor plant of embodiment 25 comprising the additional engineered attributes of embodiment 1.
Embodiment 29. An engineered ornamental indoor plant characterized in that:
   (a) at least one pathway related to diffusion and/or active transport of VOCs into the ornamental plant are modified; and
   (b) when cultivated or maintained in an environment comprising a volatile organic compound (VOC), exhibits an increased rate of air VOC removal when compared to an ornamental indoor plant that has not been modified.
Embodiment 30. The engineered ornamental indoor plant of embodiment 29 that is stably transformed with at least one expression vector from which the at least one polypeptide related to pathways regulating diffusion and/or active transport of VOCs into the ornamental plant is expressed.
Embodiment 31. The engineered ornamental indoor plant of embodiment 29 that is stably engineered to have at least one endogenous polypeptide involved in a pathway related to diffusion and/or active transport of VOCs into the ornamental plant modified.
Embodiment 32. The engineered ornamental indoor plant of embodiment 29 that is stably engineered to have at least one endogenous polypeptide involved in a pathway related to diffusion and/or active transport of VOCs into the ornamental plant knocked-out, silenced, and/or rendered hypomorphic.
Embodiment 33. The engineered ornamental indoor plant of embodiment 29 that is stably transformed with at least one expression vector from which at least one polypeptide related to pathways regulating diffusion and/or active transport of VOCs is expressed.
Embodiment 34. The engineered ornamental indoor plant of embodiment 29 that is stably engineered to have at least one endogenous polypeptide related to stomatal flux knocked-out, silenced, and/or rendered hypomorphic, wherein the at least one polypeptide Epidermal Patterning Factor 1 (EPF1) and/or Epidermal Patterning Factor 2 (EPF2).
Embodiment 35. The engineered ornamental indoor plant of embodiment 29 that is stably transformed with at least one expression vector from which at least one polypeptide related to stomatal flux is expressed, wherein the at least one polypeptide comprises Epidermal Patterning Factor-Like protein 9 (EPFL9) (STOMAGEN)
Embodiment 36. The engineered ornamental indoor plant of embodiment 29 that is stably transformed with at least one expression vector from which at least one polypeptide related to cuticle wax levels is expressed, wherein the at least one polypeptide comprises Aledehyde Decarbonylase (CER1), Fatty Acid Reductase (CER3), Beta-ketoacyl-coenzyme A Synthase, 3′-5′-exoribonuclease family protein (CER7), and/or WOOLLY.
Embodiment 37. The engineered ornamental indoor plant of embodiment 29 that is stably transformed with at least one expression vector from which at least one polypeptide related to trichome development is expressed, wherein the at least one polypeptide comprises MYB123-Like, Caprice (CPC), GLABRA1, GLABRA2, and/or GLABRA3.
Embodiment 38. The engineered ornamental indoor plant of embodiment 29 that is stably transformed with at least one expression vector from which at least one heterologous polypeptide related to active transport of VOCs is expressed, wherein the at least one polypeptide comprises an Oxalate: Formate Antiport polypeptide, Formate: Nitrite Transporter polypeptide, and/or 2FoCA-Anion Channel polypeptide.
Embodiment 39. The engineered ornamental indoor plant of embodiment 29, wherein prior to introduction to the ornamental indoor plant, the at least one polypeptide involved in a pathway related to diffusion and/or active transport of VOCs has been modified using protein evolution.
Embodiment 40. A cell or population of cells derived from the engineered ornamental indoor plant of embodiment 29.
Embodiment 41. The engineered ornamental indoor plant of embodiment 29, crossed with the engineered ornamental plant of any one of embodiments 1 or 15.
Embodiment 42. The engineered ornamental indoor plant of embodiment 3, comprising the additional engineered attributes of any one of embodiments 1 or 15.
Embodiment 43. A cell or population of cells derived from the engineered ornamental indoor plant of embodiment 3 comprising the additional engineered attributes of embodiments 1 or 15.
Embodiment 44. An engineered ornamental indoor plant characterized in that:
   (a) at least one endogenous gene encoding a protein known to function in transgene silencing has been knocked-out, silenced, and/or rendered hypomorphic.
Embodiment 45. The engineered ornamental indoor plant of embodiment 4, comprising the additional engineered attributes of any one of embodiments 1-3.
Embodiment 46. A cell or population of cells derived from the engineered ornamental indoor plant of embodiment 44 comprising the additional engineered attributes of any one of embodiments 1, 15, or 29.
Embodiment 47. The engineered ornamental indoor plant of embodiment 44, wherein the endogenous gene is RDR6.
Embodiment 48. A population of engineered microbes modified to be more amenable for VOC removal and/or metabolism when compared to a population of non-engineered microbes under otherwise comparable conditions.
Embodiment 49. The population of engineered microbes of embodiment 48, wherein the microbes are soil dwelling and comprise microbes of the species: Bacillus metanolcius, Ogataea methanolica, Pseudomonas putida, Phanerochaete chrysosporium, and/or Rugosibacter aromaticivorans.
Embodiment 50. The population of engineered microbes of embodiment 48, wherein the microbes are leaf and/or epidermal dwelling and comprise microbes of the species: Methylobacterium oryzae, Methylobacterium extorquens, and/or Paraburkholderia phytofirmans.
Embodiment 51. The population of engineered microbes of embodiment 48, wherein the microbes are leaf and/or epidermal dwelling and comprise microbes of the species: Cladophialophora immunda, Cladophialophora psammophila, Cladosporiulm sphaerospermum, Exophiala xenobiotica, Hormoconis resinae, Paecilomyces variotii, Phanerochaete chrysosporium, Picnidiella resinae, Pseudoeurotium zonatum.
Embodiment 52. The population of engineered microbes of embodiment 48, wherein the microbes are modified to metabolize formaldehyde with greater efficiency and at a greater capacity than microbes which have not been engineered.
Embodiment 53. The population of engineered microbes of embodiment 48, wherein the microbes are modified to metabolize BTEX with greater efficiency and at a greater capacity than microbes which have not been engineered.
Embodiment 54. The population of engineered microbes of embodiment 48, wherein the microbes are modified utilizing horizontal gene transfer from a heterologous microbe that has undergone directed evolution to increase formaldehyde or BTEX metabolism.
Embodiment 55. The population of engineered microbes of embodiment 48, wherein the microbes are of the species Pseudomonas putida, Methylobacterium oryzae, or Methylobacterium extorquens
Embodiment 56. The population of engineered microbes of embodiment 48, wherein the microbes are deposited on an engineered ornamental indoor plant of any one of embodiments 1, 15, 29, or 44.
Embodiment 57. The population of engineered microbes of embodiment 48, wherein the microbes are deposited and stably colonize an engineered ornamental indoor plant of any one of embodiments 1, 15, 29, or 44.
Embodiment 58. The population of engineered microbes of embodiment 48, wherein the microbes are of the strain MoCBM20.
Embodiment 59. The population of engineered microbes of embodiment 48, wherein the microbes are of the strain MePA1.
Embodiment 60. The population of engineered microbes of embodiment 48, wherein the microbes are of the strain PpF1.
Embodiment 61. The population of engineered microbes of embodiment 48, wherein the microbes are of the strain Cp110553 (CBS110553)
Embodiment 62. The population of engineered microbes of embodiment 48, wherein the microbes are of the strain Ci110551 (CBS110551).
Embodiment 63. A plant growth system comprising:
   (a) at least one container comprising at least one cavity suitable for receiving plant growth media and an engineered ornamental plant, and
   (b) at least one air flow device engineered to provide increased airflow to an engineered ornamental plant.
Embodiment 64. The plant growth system of embodiment 63, including at least one drainage system engineered to maintain a desired rhizosphere microbiome composition.
Embodiment 65. The plant growth system of embodiment 63, wherein a composition of any one of embodiments 1, 15, 29, 44 or 48 are deposited within.
Embodiment 66. The plant growth system of embodiment 63, wherein (a) and (b) are part of the same physical structure.
Embodiment 67. The plant growth system of embodiment 63, wherein the at least one container is designed to increase relative airflow and/or air exchange between the soil and/or microbiome and a surrounding environment when compared to a control plant growth system.
Embodiment 68. The plant growth system of embodiment 63, wherein the at least one container is designed to maximize relative airflow and/or air exchange between the soil and/or microbiome and a surrounding environment when compared to a control plant growth system.
Embodiment 69. A method of removing at least one VOC from an environment, the method comprising cultivating or maintaining at least one composition of any one of embodiments 1, 15, 29, 44, 48 or 63 in an environment comprising VOCs.
Embodiment 70. The method of embodiment 7, wherein the method comprises cultivating or maintaining the at least one composition of embodiments 1, 15, 29, 44, 48 or 63 for at least 1 day.
Embodiment 71. The method of embodiment 7, wherein the method comprises cultivating or maintaining at least one composition of embodiments 1, 15, 29, 44, 48 or 63 for every 100 m³ of indoor space.
Embodiment 72. A method of assessing an engineered indoor ornamental plant, microbe, plant-microbe combination, or plant-microbe-planter combination of any one of embodiments 1, 15, 29, 44, 48 or 63 comprising:
   (a) cultivating or maintaining said engineered plant in a controlled environment comprising a readily
detectable and quantifiable concentration of VOCs, and
   (b) determining the level and rate of change in VOC levels in said controlled environment.
Embodiment 73. A method of assessing a vector encoding at least one polypeptide utilized to create an engineered ornamental indoor plant of any one of embodiments 1, 15, 29, or 44 comprising:
   (a) expressing said vector in a cell, and
   (b) determining the transcriptional levels, translational levels, and molecular activity levels of said vector;
   wherein the step of determining the molecular activity of said vector comprises determining the level of VOC removal and/or metabolism relative to that achieved by an otherwise comparable reference cell under otherwise comparable conditions, which reference cell is not expressing or is not expressing to the same level of at least one polypeptide as the test cell.
Embodiment 74. A vector encoding at least one polypeptide utilized to create an engineered ornamental indoor plant of any one of embodiments 1, 15, 29, or 44.
Embodiment 75. A method of making an engineered ornamental indoor plant comprising the introduction of at least one vector encoding at least one polypeptide of any one of embodiments 1, 15, 29, or 44.
Embodiment 76. A method of making at least one vector encoding at least one polypeptide utilized to create an engineered ornamental indoor plant of any one of embodiments 1, 15, 29, or 44.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. An engineered ornamental indoor plant characterized in that:
(a) it is stably transformed with at least one expression vector;
(b) when cultivated or maintained in an environment comprising a volatile organic compound (VOC), exhibits an increased rate of air VOC removal, when compared to an ornamental indoor plant that has not been so engineered; and
(c) at least one of options (i), (ii), (iii) below applies:
(i) the at least one expression vector expresses a formaldehyde or methanol metabolism polypeptide;
(ii) the at least one expression vector expresses a heterologous benzene, toluene, ethylbenzene, or xylene (BTEX) metabolism polypeptide; and
(iii) the at least one expression vector expresses a polypeptide that regulates diffusion and/or active transport of VOCs into the ornamental plant.

2. The engineered ornamental indoor plant of claim 1, wherein
a) option (i) applies, and
b) a plurality of polypeptides expressed by the plant function in concert to convert a VOC to a usable sugar substrate.

3.-4. (canceled)

5. The engineered ornamental indoor plant of claim 2, wherein the at least one heterologous formaldehyde metabolism polypeptide comprises: 3-hexulose-6-phosphate synthase (HPS), 6-phospho-3-hexuloisomerase (PHI), dihydroxyacetone synthase (DAS), dihydroxyacetone kinase (DAK), formaldehyde dehydrogenase (FALDH), glutathione-dependent formaldehyde dehydrogenase (GSH-FALDH), glycolaldehyde synthase (GALS), acetyl-phosphate synthase (ACPS), phosphate acetyltransferase (PTA), 2-keto-4-hydroxybutyrate aldolase (KHB), branched-chain alpha-keto acid decarboxylase (KDC), pyruvate decarboxylase (PDC), NADH-dependent 1,3-PDO oxidoreductase (DhaT), non-specific NADPH-dependent alcohol dehydrogenase (YqhD), serine aldolase (SAL), threonine aldolase (LtaE), serine deaminase (SDA), 4-hydroxy-2-oxobutanoate (HOB) aldolase (HAL), HOB aminotransferase (HAT), serine hydroxymethyltransferase 1 mitochondrial (SHM1), (S)-2-hydroxy-acid oxidase (GLO1 and/or GLO2), formate dehydrogenase (FDH), and/or formolase (FLS).

6.-14. (canceled)

15. The engineered ornamental indoor plant of claim 1, wherein
(a) option (ii) applies; and
(b) prior to introduction to the ornamental indoor plant, the at least one heterologous BTEX metabolism polypeptide has been modified using protein evolution.

16.-17. (canceled)

18. The engineered ornamental indoor plant of claim 15, wherein a plurality of polypeptides function in concert to chemically convert BTEX to a usable anabolic substrate.

19. The engineered ornamental indoor plant of claim 15, wherein the at least one heterologous BTEX metabolism polypeptide comprises: cytochrome P450 monooxygenase, O-xylene monooxygenase oxygenase subunit alpha, benzene monooxygenase oxygenase subunit, toluene-4-monooxygenase system ferredoxin-NAD (+) reductase component, toluene monooxygenase alpha subunit, aromatic ring-hydroxylating dioxygenase subunit alpha, hydroxylase alpha subunit, phenylalanine hydroxylase, benzene 1,2-dioxygenase, cis-1,2-dihydrobenzene-1,2-diol dehydrogenase, toluene methyl-monooxygenase, aryl-alcohol dehydrogenase, benzaldehyde dehydrogenase (NAD+), and/or benzaldehyde dehydrogenase (NADP+), wherein the at least one heterologous BTEX metabolism polypeptide alters
(a) the benzene and/or ethylbenzene metabolism pathway;
(b) the toluene and/or xylene metabolism pathway; or
(c) a combination of (a) and (b).

20.-21. (canceled)

22. The engineered ornamental indoor plant of claim 15, wherein the at least one heterologous BTEX metabolism polypeptide alters the phenol and/or phenol (like) metabolism pathway, wherein the heterologous polypeptides comprise phenol hydroxylase component phP, phenol hydroxylase, and/or uncharacterized protein A4U43_C04F5180.

23. The engineered ornamental indoor plant of claim 15, wherein the at least one heterologous BTEX metabolism polypeptide alters the catechol and/or catechol (like) metabolism pathway, wherein the heterologous polypeptides comprise 3-isopropylcatechol-2,3-dioxygenase, metapyrocatechase, extradiol dioxygenase, catechol 2,3-dioxygenase, and/or catechol 1,2-dioxygenase.

24.-28. (canceled)

29. The engineered ornamental indoor plant of claim 1, wherein
(a) option (iii) applies; and
(b) at least one pathway related to diffusion and/or active transport of VOCs into the ornamental plant is modified,
wherein prior to introduction to the ornamental indoor plant, the at least one polypeptide involved in a pathway related to diffusion and/or active transport of VOCs has been modified using protein evolution.

30.-33. (canceled)

34. The engineered ornamental indoor plant of claim 29, wherein the engineered ornamental indoor plant is stably engineered to have at least one endogenous polypeptide related to stomatal flux knocked-out, silenced, and/or rendered hypomorphic, wherein the at least one endogenous polypeptide is an Epidermal Patterning Factor 1 (EPF1) and/or Epidermal Patterning Factor 2 (EPF2).

35. The engineered ornamental indoor plant of claim 29, wherein at least one of the following is expressed from the at least one expression vector:
(a) at least one polypeptide related to stomatal flux is expressed, wherein the at least one polypeptide comprises Epidermal Patterning Factor-Like protein 9 (EPFL9) (STOMAGEN);
(b) at least one polypeptide related to cuticle wax levels is expressed, wherein the at least one polypeptide related to cuticle wax levels comprises Aledehyde Decarbonylase (CER1), Fatty Acid Reductase (CER3), Beta-ketoacyl-coenzyme A Synthase, 3′-5′-exoribonuclease family protein (CER7), and/or WOOLLY;
(c) at least one polypeptide related to trichome development is expressed, wherein the at least one polypeptide related to trichome development comprises MYB123-Like, Caprice (CPC), GLABRA1, GLABRA2, and/or GLABRA3; and
(d) at least one heterologous polypeptide related to active transport of VOCs is expressed, wherein the at least one heterologous polypeptide comprises an Oxalate: Formate Antiport polypeptide, Formate: Nitrite Transporter polypeptide, and/or 2FoCA-Anion Channel polypeptide.

36.-43. (canceled)

44. An engineered ornamental indoor plant characterized in that:
(a) at least one endogenous gene encoding a protein known to function in transgene silencing has been knocked-out, silenced, and/or rendered hypomorphic.

45.-46. (canceled)

47. The engineered ornamental indoor plant of claim 44, wherein the endogenous gene is RDR6.

48. A population of engineered microbes modified to be more amenable for VOC removal and/or metabolism when compared to a population of non-engineered microbes under otherwise comparable conditions,
wherein the population of non-engineered microbes is selected from:
(a) a first population of engineered microbes modified from a reference strain of a species selected from Bacillus metanolcius, Ogataea methanolica, Pseudomonas putida, Phanerochaete chrysosporium, or Rugosibacter aromaticivorans;
(b) a second population of engineered microbes modified from a second reference strain of a species selected from Methylobacterium oryzae, Methylobacterium extorquens, Paraburkholderia phytofirmans; and
(c) a third population of engineered microbes modified from a third reference strain of a species selected from Cladophialophora immunda, Cladophialophora psammophila, Cladosporiulm sphaerospermum, Exophiala xenobiotica, Hormoconis resinae, Paecilomyces variotii, Phanerochaete chrysosporium, Picnidiella resinae, or Pseudoeurotium zonatum.

49.-62. (canceled)

63. A plant growth system comprising:
(a) at least one container comprising at least one cavity suitable for receiving plant growth media and an engineered ornamental plant that is designed to increase or maximize relative airflow and/or air exchange between the soil and/or microbiome and a surrounding environment when compared to a control plant growth system;
(b) at least one air flow device engineered to provide increased airflow to an engineered ornamental plant;
(c) at least one drainage system coupled to or integrated into the at least one container and engineered to maintain a desired rhizosphere microbiome composition; or
(d) a combination of (a), (b), and (c),
wherein (a) and (b) are part of the same structure.

64. (canceled)

65. The plant growth system of claim 63, wherein the system comprises an engineered ornamental indoor plant that, when cultivated or maintained in an environment comprising a volatile organic compound (VOC), exhibits an increased rate of air VOC removal when compared to an ornamental indoor plant that has not been so engineered, the engineered ornamental indoor plant comprising at least one additional characteristic chosen from (a) through (d) below:
(a) it is stably transformed with at least one expression vector that is used to express at least one heterologous formaldehyde and/or methanol metabolism polypeptide;
(b) it is stably transformed with at least one expression vector that is used to express at least one heterologous benzene, toluene, ethylbenzene, or xylene (BTEX) metabolism polypeptide;
(c) it is stably transformed with at least one expression vector that is used to express at least one polypeptide related to pathways regulating diffusion and/or active transport of VOCs into the ornamental plant; and
(d) it comprises at least one endogenous gene encoding a protein known to function in transgene silencing that has been knocked-out, silenced, and/or rendered hypomorphic.

66. The plant growth system of claim 65, comprising a composition of engineered microbes deposited into the plant growth media and/or on the engineered ornamental indoor plant, wherein the composition comprises one or more engineered microbe populations selected from:
(a) a first population of engineered microbes modified from a reference strain of a species selected from Bacillus metanolcius, Ogataea methanolica, Pseudomonas putida, Phanerochaete chrysosporium, or Rugosibacter aromaticivorans;
(b) a second population of engineered microbes modified from a second reference strain of a species selected from Methylobacterium oryzae, Methylobacterium extorquens, Paraburkholderia phytofirmans; and
(c) a third population of engineered microbes modified from a third reference strain of a species selected from Cladophialophora immunda, Cladophialophora psammophila, Cladosporiulm sphaerospermum, Exophiala xenobiotica, Hormoconis resinae, Paecilomyces variotii, Phanerochaete chrysosporium, Picnidiella resinae, or Pseudoeurotium zonatum, wherein the engineered microbes are characterized by one or both of greater VOC removal and greater VOC metabolism when compared to the respective reference strain.

67.-71. (canceled)

72. A method of assessing a capacity of the engineered indoor ornamental plant of claim 1 to remove VOC from a controlled environment, the method comprising:
(a) cultivating or maintaining said engineered plant in the controlled environment, the controlled environment comprising a readily detectable and quantifiable concentration of VOCs; and
(b) determining the level and rate of change in VOC levels in the controlled environment.

73.-76. (canceled)
